SEQUENCE LISTING (4 sequences)

SEQ ID NO: 1 - RNA, 244 nt, synthetic construct
  misc_feature 1..244: note = Description of Artificial Sequence: Synthetic polynucleotide
  source 1..244: mol_type = other RNA
  modified_base join(1,5,9,13,17,21,25,29,33,37,41,45,49,53,57,61,65,69,73,77,81,87,93,99,105,111,117,123,129,135,141,147,153,159,165,169,173,177,181,185,189,193,197,201,205,209,213,217,221,225,229,233,237,241): mod_base = OTHER; note = uracil
tcagtcagtcagtcagtcagtcagtcagtcagtcagtcagtcagtcagtcagtcagtcagtcagtcagtcagtcagtcagtccaagtccaagtccaagtccaagtccaagtccaagtccaagtccaagtccaagtccaagtccaagtccaagtccaagtccaagtcagtcagtcagtcagtcagtcagtcagtcagtcagtcagtcagtcagtcagtcagtcagtcagtcagtcagtcagtcag

SEQ ID NO: 2 - DNA, 22 bp, synthetic construct
  misc_feature 1..22: note = Description of Artificial Sequence: Synthetic primer
  source 1..22: mol_type = other DNA
catccagcaaatacaccatcca

SEQ ID NO: 3 - DNA, 28 bp, synthetic construct
  misc_feature 1..28: note = Description of Artificial Sequence: Synthetic primer
  source 1..28: mol_type = other DNA
ttctgcacatcataattaggagtatcaa

SEQ ID NO: 4 - DNA, 15 bp, synthetic construct
  source 1..15: mol_type = other DNA
  misc_feature 1..15: note = Description of Artificial Sequence: Synthetic probe
cggagcacaggagat

(19) United States
(12) Patent Application Publication
(10) Pub. No.: US 2023/0295201 A1
(43) Pub. Date: Sep. 21, 2023

(54) ANTIVIRAL COMPOUNDS

(71) Applicant: Gilead Sciences, Inc., Foster City, CA (US)

(72) Inventors: Daniel H. Byun, Foster City, CA (US); Gregory F. Chin, San Francisco, CA (US); Byoung-kwon Chun, Pleasanton, CA (US); Michael O. Clarke, Redwood City, CA (US); Bindu Goyal, Fremont, CA (US); Hon C. Hui, San Mateo, CA (US); Petr Jansa, Foster City, CA (US); Richard L. Mackman, Millbrae, CA (US); Michael R. Mish, Foster City, CA (US); Dustin S. Siegel, Half Moon Bay, CA (US); David Sperandio, Palo Alto, CA (US); Hai Yang, San Mateo, CA (US); Lijun Zhang, Los Altos Hills, CA (US)

(21) Appl. No.: 18/171,950

(22) Filed: Feb. 21, 2023

Related U.S. Application Data
(63) Continuation of application No. 17/176,497, filed on Feb. 16, 2021, now abandoned.
(60) Provisional application No. 62/978,192, filed on Feb. 18, 2020.

(51) Int. Cl. C07F 9/6561; A61K 31/706

(52) U.S. Cl. CPC C07F 9/6561; A61K 31/706

(57) ABSTRACT

The present disclosure provides compounds for treating a variety of diseases, such as respiratory syncytial virus (RSV), HRV, hMPV, ebola, Zika, West Nile, Dengue, and HCV.

### CROSS-REFERENCES TO RELATED APPLICATIONS

This application is a continuation of U.S. application Ser. No. 17/176,497, filed 16 Feb. 2021, which claims the benefit of priority to U.S. Provisional Patent Application No. 62/978,192, filed 18 Feb. 2020 and titled “ANTIVIRAL COMPOUNDS,” the entireties of which are incorporated herein by reference.

### SEQUENCE LISTING

The material in the accompanying sequence listing is hereby incorporated by reference in its entirety. The accompanying file, named “052838-515C01US Sequence Listing_ST26.xml” was created on Feb. 17, 2023 and is 7,145 bytes.

### BACKGROUND OF THE INVENTION

Pneumoviridae viruses are negative-sense, single-stranded, RNA viruses that are responsible for many prevalent human and animal diseases. The Pneumoviridae family of viruses includes human respiratory syncytial virus (HRSV) and human metapneumovirus. Almost all children will have had an HRSV infection by their second birthday. HRSV is the major cause of lower respiratory tract infections in infancy and childhood with 0.5% to 2% of those infected requiring hospitalization.

No vaccine to prevent HRSV infection is currently available. The monoclonal antibody palivizumab is available for immunoprophylaxis, but its use is restricted to infants at high risk, e.g., premature infants or those with either congenital heart or lung disease, and the cost for general use is often prohibitive. In addition, nucleoside analog ribavirin has been approved as the only antiviral agent to treat HRSV infections but has limited efficacy. Therefore, there is a need for anti-Pneumoviridae therapeutics.

Examples of pyrrolo[2,3-d]pyrimidine compounds useful for treating viral infections are described in U.S. 2012/0009147 A1 (Cho et al.), U.S. 2012/0020921 A1 (Cho et al.), WO 2008/089105 A2 (Babu et al.), WO 2008/141079 A1 (Babu et al.), WO 2009/132135 A1 (Butler et al.), WO 2010/002877 A2 (Francom), WO 2011/035231 A1 (Cho et al.), WO 2011/035250 A1 (Butler et al.), WO 2011/150288 A1 (Cho et al.), WO 2012/012465 (Cho et al.), WO 2012/012776 A1 (Mackman et al.), WO 2012/037038 (Clarke et al.), WO 2012/087596 A1 (Delaney et al.), and WO 2012/142075 A1 (Girijavallabhan et al.).

Thus, there is a need for compositions and methods for treating Pneumoviridae viral infections, such as HRSV infections, that are effective and have acceptable toxicity profiles, Flaviviridae infections, including dengue, and EBOV infections. The present disclosure addresses these and other needs.

### BRIEF SUMMARY OF THE INVENTION

In one embodiment, the present disclosure provides a compound of Formula (Ia):

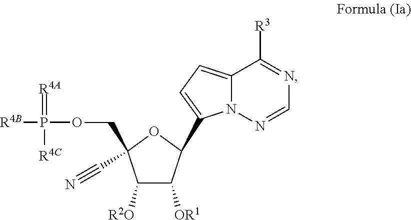

or a pharmaceutically acceptable salt thereof, wherein:
R¹ and R² are each independently H or —C(O)R^{1A}, wherein R^{1A} is C₁₋₆ alkyl, wherein at least one of R¹ and R² is H;
or R¹ and R² are combined to form —C(O)— or —C(R^{2A})(R^{2B})—, wherein each R^{2A} and R^{2B} is independently H, C₁₋₆ alkyl or C₁₋₆ alkoxy;
R³ is —N(H)(R^{3A});
R^{3A} is H or —C(O)R^{3A1}, wherein R^{3A1} is C₁₋₁₈ alkyl optionally substituted with —NH₂;
R^{4A} is O or S; and
R^{4B} and R^{4C} are each independently:
   (A) —OH;
   (B) —OR^{4B1}, wherein
      R^{4B1} is C₁₋₆ alkyl optionally substituted with 1 to 3 R^{4B2} groups, C₁₋₆ haloalkyl, C₃₋₈ cycloalkyl, C₆₋₁₂ aryl, or a 5 to 6 membered heteroaryl having 1 to 3 heteroatoms each independently selected from N, O or S, wherein
         each R^{4B2} group is independently C₁₋₆ alkoxy, —S—R^{4B3}, or —S(O)₂—R^{4B3}, and
         each R^{4B3} group is independently C₁₋₆ alkyl;

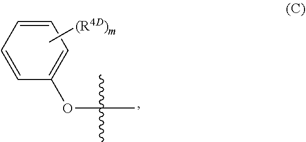

wherein
   subscript m is 0, 1, 2, 3, 4, or 5; and
   each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein
      each R^{4D1} group is independently —NH₂ or —C(O)OR^{4D3},
      each R^{4D2} is independently C₁₋₃ alkoxy, and
      each R^{4D3} is independently C₁₋₃ alkyl;

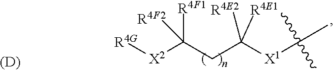

wherein
   X¹ and X² are each independently —O— or —N(R^{4H})—;
   R^{4E1} and R^{4E2} are each independently H, C₁₋₆ alkyl optionally substituted with 1 to 3 R^{4E3} groups, or C₃₋₆ cycloalkyl, wherein
      each R^{4E3} group is independently-C(O)OR^{4E4}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and
      each R^{4F4} group is independently C₁₋₆ alkyl;
   or R^{4E1} and R^{4E2} are combined with the atom to which they are attached to form a C₃₋₆ cycloalkyl;
   R^{4F1} and R^{4F2} are each H or together are oxo;
   R^{4G} is C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

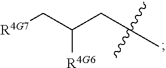

each R^{4G1} is independently —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl;
each R^{4G2} is independently C₁₋₆ alkyl, C₁₋₆ alkoxy, halogen, C₁₋₃ haloalkyl, —OH, —NH₂, or C₆₋₁₂ aryl;
each R^{4G3} is independently C₁₋₆ alkyl, halogen, C₁₋₃ haloalkyl, oxo, —C(O)R^{4G5}, or —C(O)OR^{4G5};
each R^{4G4} is independently C₁₋₆ alkyl, C₇₋₁₈ alkyl or C₃₋₈ cycloalkyl, wherein the C₁₋₆ alkyl is optionally substituted with OH, NH₂, or —NHC(O)OR^{4G5}, and wherein the cycloalkyl is optionally substituted with C₁₋₆ alkyl;
each R^{4G5} is independently C₁₋₆ alkyl;
R^{4G6} and R^{4G7} are each independently H or —OR^{4G11}, wherein at least one of R^{4G6} and R^{4G7} is —OR^{4G11};
each R^{4G8} is independently H or C₁₋₆ alkyl;
each R^{4G9} is independently C₁₋₆ alkyl, halogen, C₁₋₃ haloalkyl, or —NH₂;
each R^{4G10} is independently C₁₋₆ alkyl, C₁₋₃ haloalkyl, or oxo;
each R^{4G11} is independently C₁₀₋₁₈ alkyl or benzyl;
R^{4H} is H;
or R^{4E1} and R^{4H} are combined with the atoms to which they are attached to form a 5 to 6 membered heterocyclyl having 1 to 2 additional heteroatoms selected from N, O and S; and
subscript n is 0 or 1; or
(E) —(OP(O)(OH))₁₋₂—OH; or

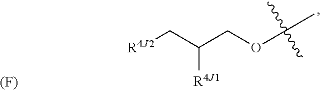

wherein
   R^{4J1} and R^{4J2} are each independently H, —OR^{4J3} or —OC(O)R^{4J3}, wherein at least one of R^{4J1} and R^{4J2} is —OR^{4J3} or —OC(O)R^{4J3},
      each R^{4J3} is independently C₁₋₁₈ alkyl, C₂₋₆ alkenyl, or benzyl, and at least one R^{4J3} is C₁₀₋₁₈ alkyl;
alternatively, R² and R^{4C} are combined with the atoms to which they are attached to form a six-membered ring, and R¹ is H or —C(O)R^{1A}, wherein R^{1A} is C₁₋₆ alkyl,
with the proviso that when the compound of Formula (Ia) has the formula:

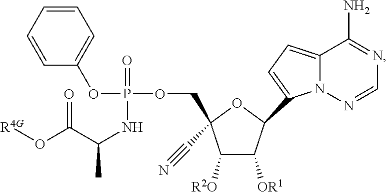

and R^{4G} is ethyl or 2-ethylbutyl, then one of R¹ and R² is —C(O)R^{1A}, or R¹ and R² are combined to form —C(O)— or —C(R^{2A})(R^{2B})—,
with the proviso that the compound of Formula (Ia) does not have the structure:

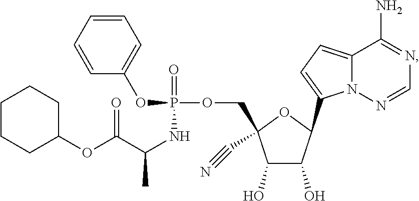 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]

and with the proviso that when the compound of Formula (Ia) has the formula:

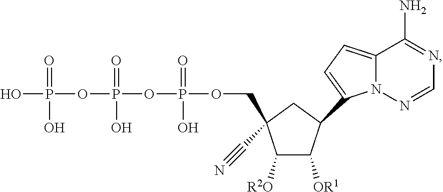

then one of R¹ and R² is —C(O)R^{1A}, or R¹ and R² are combined to form —C(O)— or —C(R^{2A})(R^{2B})—.

In another embodiment, the present disclosure provides a pharmaceutical formulation comprising a pharmaceutically effective amount of a compound of the present disclosure, or a pharmaceutically acceptable salt thereof, and a pharmaceutically acceptable carrier or excipient.

In another embodiment, the present disclosure provides a method of treating a Pneumoviridae virus infection in a human in need thereof, the method comprising administering to the human a therapeutically effective amount of a compound of the present disclosure, or a pharmaceutically acceptable salt thereof.

In another embodiment, the present disclosure provides a method of treating a Picornaviridae virus infection in a human in need thereof, the method comprising administering to the human a therapeutically effective amount of a compound of the present disclosure, or a pharmaceutically acceptable salt thereof.

In another embodiment, the present disclosure provides a method of treating a Flaviviridae virus infection in a human in need thereof, the method comprising administering to the human a therapeutically effective amount of a compound of the present disclosure, or a pharmaceutically acceptable salt thereof.

In another embodiment, the present disclosure provides a method of treating a Filoviridae virus infection in a human in need thereof, the method comprising administering to the human a therapeutically effective amount of a compound of the present disclosure, or a pharmaceutically acceptable salt thereof.

In another embodiment, the present disclosure provides a method for manufacturing a medicament for treating a Pneumoviridae virus infection in a human in need thereof, characterized in that a compound of the present disclosure, or a pharmaceutically acceptable salt thereof, is used.

In another embodiment, the present disclosure provides a method for manufacturing a medicament for treating a Picornaviridae virus infection in a human in need thereof, characterized in that a compound of the present disclosure, or a pharmaceutically acceptable salt thereof, is used.

In another embodiment, the present disclosure provides a method for manufacturing a medicament for treating a Flaviviridae virus infection in a human in need thereof, characterized in that a compound of the present disclosure, or a pharmaceutically acceptable salt thereof, is used.

In another embodiment, the present disclosure provides a method for manufacturing a medicament for treating a Filoviridae virus infection in a human in need thereof, characterized in that a compound of the present disclosure, or a pharmaceutically acceptable salt thereof, is used.

In another embodiment, the present disclosure provides use of a compound of the present disclosure, or a pharmaceutically acceptable salt thereof, for the manufacture of a medicament for the treatment of a Pneumoviridae virus infection in a human.

In another embodiment, the present disclosure provides use of a compound of the present disclosure, or a pharmaceutically acceptable salt thereof, for the manufacture of a medicament for the treatment of a Picornaviridae virus infection in a human.

In another embodiment, the present disclosure provides use of a compound of the present disclosure, or a pharmaceutically acceptable salt thereof, for the manufacture of a medicament for the treatment of a Flaviviridae virus infection in a human.

In another embodiment, the present disclosure provides use of a compound of the present disclosure, or a pharmaceutically acceptable salt thereof, for the manufacture of a medicament for the treatment of a Filoviridae virus infection in a human.

In another embodiment, the present disclosure provides the compound of the present disclosure, or a pharmaceutically acceptable salt thereof, for use in the treatment of a Pneumoviridae virus infection in a human in need thereof.

In another embodiment, the present disclosure provides the compound of the present disclosure, or a pharmaceutically acceptable salt thereof, for use in the treatment of a Picornaviridae virus infection in a human in need thereof.

In another embodiment, the present disclosure provides the compound of the present disclosure, or a pharmaceutically acceptable salt thereof, for use in the treatment of a Flaviviridae virus infection in a human in need thereof.

In another embodiment, the present disclosure provides the compound of the present disclosure, or a pharmaceutically acceptable salt thereof, for use in the treatment of a Filoviridae virus infection in a human in need thereof.

In another embodiment, the present disclosure provides a method for the treatment or prophylaxis of an exacerbation of a respiratory condition by a viral infection in a human in need thereof, the method comprising administering to the human a therapeutically effective amount of a compound of the present disclosure, or a pharmaceutically acceptable salt thereof, wherein the respiratory condition is chronic obstructive pulmonary disease.

In another embodiment, the present disclosure provides a method for manufacturing a medicament for the treatment or prophylaxis of an exacerbation of a respiratory condition by a viral infection in a human in need thereof, characterized in that a compound of the present disclosure, or a pharmaceutically acceptable salt thereof, is used, wherein the respiratory condition is chronic obstructive pulmonary disease.

In another embodiment, the present disclosure provides use of a compound of the present disclosure, or a pharmaceutically acceptable salt thereof, for the manufacture of a medicament for the treatment or prophylaxis of an exacerbation of a respiratory condition by a viral infection in a human, wherein the respiratory condition is chronic obstructive pulmonary disease.

In another embodiment, the present disclosure provides the compound of the present disclosure, or a pharmaceutically acceptable salt thereof, for use in the treatment or prophylaxis of an exacerbation of a respiratory condition by a viral infection in a human in need thereof, wherein the respiratory condition is chronic obstructive pulmonary disease.

### DETAILED DESCRIPTION OF THE INVENTION

### I. General

The present disclosure provides 2′,3′-dihydroxy-4′-cyano nucleoside and monoester compounds for the treatment of viral infections, such as Ebola, zika, West Nile, Yellow Fever, Dengue, HCV, RSV, and others.

### II. Definitions

“Alkyl” is a linear or branched saturated monovalent hydrocarbon. For example, an alkyl group can have 1 to 18 carbon atoms (i.e., C₁₋₁₈ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include heptyl, octyl, nonyl, decyl, undecyl, dodecyl, pentadecyl, hexadecyl, heptadecyl and octadecyl.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxy-alkoxy” refers an alkoxy group linked to a second alkoxy group which is linked to the remainder of the compound. Alkoxy is as defined above, and can include, but is not limited to, methoxy-methoxy (CH₃OCH₂O—), methoxy-ethoxy (CH₃OCH₂CH₂O—) and others.

“Hydroxy” refers to —OH.

“Halo” or “halogen” as used herein refers to fluoro (—F), chloro (—Cl), bromo (—Br) and iodo (—I).

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocyles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular heteroatom selected from oxygen, nitrogen, and sulfur). Unless otherwise specified, a heterocyclyl group has from 3 to about 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like.

“Aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂-4, C₂-5, C₂-6, C₃-4, C₃-5, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, and triazolyl.

A “compound of the present disclosure” includes compounds disclosed herein, for example a compound of the present disclosure includes compounds of Formula (Ia), (Ib), (Ic), (Id), (Ie), (If), (Ig), (Ih) and (Ii), including the compounds of the Examples.

“Pharmaceutically effective amount” refers to an amount of a compound of the present disclosure in a formulation or combination thereof, that provides the desired therapeutic or pharmaceutical result.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Prophylaxis” refers to preventing or retarding the progression of clinical illness in patients suffering from a viral infection.

“Therapeutically effective amount” or “effective amount” as used herein refers to an amount that is effective to elicit the desired biological or medical response, including the amount of a compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possess the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly understood by one of ordinary skill in the art. A dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

### III. Compounds

The present disclosure provides compounds of Formula (Ia), (Ib), (Ic), (Id), (Ie), (If), (Ig), (Ih), and (Ii).

In some embodiments, the present disclosure provides a compound of Formula (Ia):

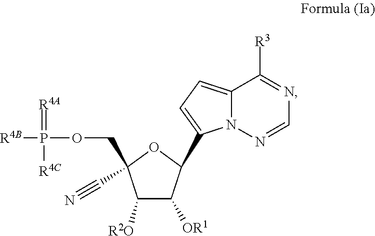

or a pharmaceutically acceptable salt thereof, wherein:
R¹ and R² are each independently H or —C(O)R^{1A}, wherein R^{1A} is C₁₋₆ alkyl, wherein at least one of R¹ and R² is H;
or R¹ and R² are combined to form —C(O)— or —C(R^{2A})(R^{2B})—, wherein each R^{2A} and R^{2B} is independently H, C₁₋₆ alkyl or C₁₋₆ alkoxy;
R³ is —N(H)(R^{3A});
R^{3A} is H or —C(O)R^{3A1}, wherein R^{3A1} is C₁₋₁₈ alkyl optionally substituted with —NH₂;
R^{4A} is O or S; and
R^{4B} and R^{4C} are each independently:
   (A) —OH;
   (B) —OR^{4B1}, wherein
      R^{4B1} is C₁₋₆ alkyl optionally substituted with 1 to 3 R^{4B2} groups, C₁₋₆ haloalkyl, C₃₋₈ cycloalkyl, C₆₋₁₂ aryl, or a 5 to 6 membered heteroaryl having 1 to 3 heteroatoms each independently selected from N, O or S, wherein
         each R^{4B2} group is independently C₁₋₆ alkoxy, —S—R^{4B3}, or —S(O)₂—R^{4B3} and
         each R^{4B3} group is independently C₁₋₆ alkyl;

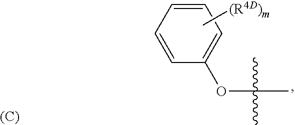

wherein
   subscript m is 0, 1, 2, 3, 4, or 5; and
   each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein
      each R^{4D1} group is independently —NH₂ or —C(O)OR^{4D3},
      each R^{4D2} is independently C₁₋₃ alkoxy, and
      each R^{4D3} is independently C₁₋₃ alkyl;

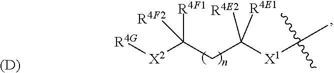

wherein
   X¹ and X² are each independently —O— or —N(R^{4H})—
   R^{4E1} and R^{4E2} are each independently H, C₁₋₆ alkyl optionally substituted with 1 to 3 R^{4E3} groups, or C₃₋₆ cycloalkyl, wherein
      each R^{4E3} group is independently-C(O)OR^{4E4}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and
      each R^{4F4} group is independently C₁₋₆ alkyl;
   or R^{4E1} and R^{4E2} are combined with the atom to which they are attached to form a C₃₋₆ cycloalkyl;
   R^{4F1} and R^{4F2} are each H or together are oxo;
   R^{4G} is C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

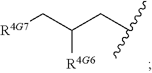

each R^{4G1} is independently —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl;
each R^{4G2} is independently C₁₋₆ alkyl, C₁₋₆ alkoxy, halogen, C₁₋₃ haloalkyl, —OH, —NH₂, or C₆₋₁₂ aryl;
each R^{4G3} is independently C₁₋₆ alkyl, halogen, C₁₋₃ haloalkyl, oxo, —C(O)R^{4G5}, or —C(O)OR^{4G5};
each R^{4G4} is independently C₁₋₆ alkyl, C₇₋₁₈ alkyl or C₃₋₈ cycloalkyl, wherein the C₁₋₆ alkyl is optionally substituted with OH, NH₂, or —NHC(O)OR^{4G5}, and wherein the cycloalkyl is optionally substituted with C₁₋₆ alkyl;
each R^{4G5} is independently C₁₋₆ alkyl;
R^{4G6} and R^{4G7} are each independently H or —OR^{4G11}, wherein at least one of R^{4G6} and R^{4G7} is —OR^{4G11};
each R^{4G6} is independently H or C₁₋₆ alkyl;
each R^{4G9} is independently C₁₋₆ alkyl, halogen, C₁₋₃ haloalkyl, or —NH₂;
each R^{4G10} is independently C₁₋₆ alkyl, C₁₋₃ haloalkyl, or oxo;
each R^{4G11} is independently C₁₀₋₁₈ alkyl or benzyl;
R^{4H} is H;
or R^{4E1} and R^{4H} are combined with the atoms to which they are attached to form a 5 to 6 membered heterocyclyl having 1 to 2 additional heteroatoms selected from N, O and S; and
subscript n is 0 or 1; or
(E) —(OP(O)(OH))₁₋₂—OH; or

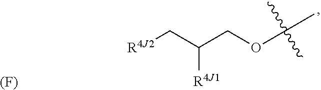

wherein
   R^{4J1} and R^{4J2} are each independently H, —OR^{4J3} or —OC(O)R^{4J3}, wherein at least one of R^{4J1} and R^{4J2} is —OR^{4J3} or —OC(O)R^{4J3},
      each R^{4J3} is independently C₁₋₁₈ alkyl, C₂₋₆ alkenyl, or benzyl, and at least one R^{4J3} is C₁₀₋₁₈ alkyl;
alternatively, R² and R^{4C} are combined with the atoms to which they are attached to form a six-membered ring, and R¹ is H or —C(O)R^{1A}, wherein R^{1A} is C₁₋₆ alkyl,
with the proviso that when the compound of Formula (Ia) has the formula:

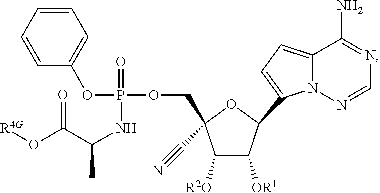

and R^{4G} is ethyl or 2-ethylbutyl, then one of R¹ and R² is —C(O)R^{1A}, or R¹ and R² are combined to form —C(O)— or —C(R^{2A})(R^{2B})—.
with the proviso that the compound of Formula (Ia) does not have the structure:

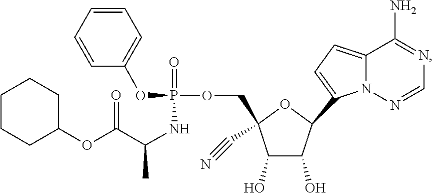 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]

and with the proviso that when the compound of Formula (Ia) has the formula:

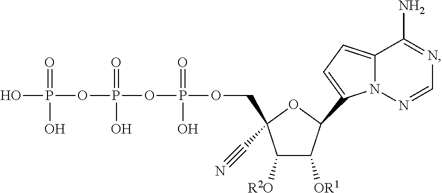

then one of R¹ and R² is —C(O)R^{1A}, or R¹ and R² are combined to form —C(O)— or —C(R^{2A})(R^{2B})—.

In some embodiments, the compound can be represented by Formula (Ia), or a pharmaceutically acceptable salt thereof, with the proviso that the compound of Formula (Ia) does not have the structure:

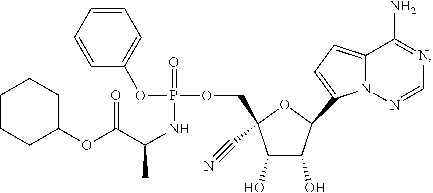 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

In some embodiments, the compound can be represented by Formula (Ia), or a pharmaceutically acceptable salt thereof, with the proviso that when the compound of Formula (Ia) has the formula:

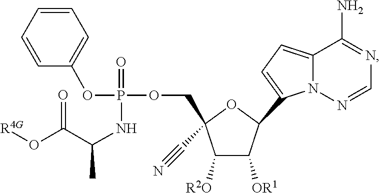

and R^{4G} is ethyl, 2-ethylbutyl or cyclohexyl, then one of R¹ and R² is —C(O)R^{1A}, or R¹ and R² are combined to form —C(O)— or —C(R^{2A})(R^{2B})—.

In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² are each independently H or —C(O)R^{1A}, wherein R^{1A} is C₁₋₆ alkyl, wherein at least one of R¹ and R² is H; or R¹ and R² are combined to form —C(O)— or —C(R^{2A})(R^{2B})—, wherein each R^{2A} and R^{2B} is independently H, C₁₋₆ alkyl or C₁₋₆ alkoxy. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)R^{1A}, wherein R^{1A} is C₁₋₆ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)R^{1A}, wherein R^{1A} is C₁₋₆ alkyl, and R² can be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —C(O)— or —C(R^{2A})(R^{2B})—, wherein each R^{2A} and R^{2B} is independently H, C₁₋₆ alkyl or C₁₋₆ alkoxy. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —C(O)— or —C(R^{2A})(R^{2B})—, wherein each R^{2A} and R^{2B} is independently H, C₁₋₄ alkyl or C₁₋₃ alkoxy.

In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² are each independently H or —C(O)R^{1A}, wherein R^{1A} is methyl, ethyl, n-propyl, iso-propyl, n-butyl, iso-butyl, sec-butyl or t-butyl, wherein at least one of R¹ and R² is H; or R¹ and R² are combined to form —C(O)—, —C(Me)₂- or —CH(OEt)-. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)R^{1A}, wherein R^{1A} is methyl, ethyl, n-propyl, iso-propyl, n-butyl, iso-butyl, sec-butyl or t-butyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)R^{1A}, wherein R^{1A} is methyl, ethyl, n-propyl, iso-propyl, n-butyl, iso-butyl, sec-butyl or t-butyl, and R² can be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —C(O)—, —C(Me)₂- or —CH(OEt)-.

In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can each be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² are each independently H or —C(O)R^{1A}, wherein R^{1A} is ethyl, iso-propyl or t-butyl, wherein at least one of R¹ and R² is H; or R¹ and R² are combined to form —C(O)—, —C(Me)₂- or —CH(OEt)-. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)R^{1A}, wherein R^{1A} is ethyl, iso-propyl or t-butyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)R^{1A}, wherein R^{1A} is ethyl, iso-propyl or t-butyl, and R² can be H.

In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)-ethyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)-iso-propyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H and R² can be —C(O)-t-butyl. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-ethyl and R² can be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-iso-propyl and R² can be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-t-butyl and R² can be H. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —C(O)—. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —C(Me)₂-. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —CH(OEt)-.

In some embodiments, the compound can be represented by Formula (Ia), (Ib), (Ic), (Id), (Ie), (If), (Ig), (Ih) or (Ii), or a pharmaceutically acceptable salt thereof, wherein R³ is —N(H)(R^{3A}); R^{3A} is H or —C(O)R^{3A1}, wherein R^{3A1} is C₁₋₁₈ alkyl optionally substituted with —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R³ is —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R³ is —NHC(O)R^{3A1}, wherein R^{3A1} is C₁₋₁₈ alkyl optionally substituted with —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R³ is —NHC(O)R^{3A1}, wherein R^{3A1} is C₁₋₁₈ alkyl optionally substituted with —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R^{3A1} can be methyl, ethyl, n-propyl, iso-propyl, n-butyl, iso-butyl, sec-butyl, tert-butyl, n-pentyl, neopentyl, hexyl, heptyl, octyl, nonyl, decyl, undecyl, dodoecyl, hexadecyl, or octadecyl, each optionally substituted with —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R^{3A1} can be n-propyl, iso-propyl, iso-butyl, heptyl, or dodoecyl, each optionally substituted with —NH₂. In some embodiments, the compound can be represented by Formula (Ia), (Ib), (Ic), (Id), (Ie), (If), (Ig), (Ih) or (Ii), or a pharmaceutically acceptable salt thereof, wherein R^{3A1} can be n-propyl, iso-propyl, heptyl, or dodoecyl. In some embodiments, the compound can be represented by Formula (Ia), (Ib), (Ic), (Id), (Ie), (If), (Ig), (Ih) or (Ii), or a pharmaceutically acceptable salt thereof, wherein R^{3A1} can be 1-aminoiso-butyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4A} can be O or S. In some embodiments, the compound can be represented by Formula (Ia), (Ib), (Ic), (Id), (Ie), (If), (Ig), (Ih) or (Ii), or a pharmaceutically acceptable salt thereof, wherein R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R^{4A} can be S.

In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can each independently be H or —C(O)R^{1A}, wherein R^{1A} can be ethyl, iso-propyl or t-butyl, wherein at least one of R¹ and R² can be H; or R¹ and R² are combined to form —C(O)—, —C(Me)₂- or —CH(OEt)-; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H; R² can be —C(O)-ethyl; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H; R² can be —C(O)-iso-propyl; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be H; R² can be —C(O)-t-butyl; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-ethyl; R² can be H; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-iso-propyl; R² can be H; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ can be —C(O)-t-butyl; R² can be H; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹; R² can be combined to form —C(O)—; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹; R² can be combined to form —C(Me)₂-; R³ can be NH₂; and R^{4A} can be O. In some embodiments, the compound can be represented by Formula (Ia), (Ib), (Ic), (Id), (Ie), (If), (Ig), (Ih) or (Ii), or a pharmaceutically acceptable salt thereof, wherein R¹ and R² can be combined to form —CH(OEt)-; R³ can be NH₂; and R^{4A} can be O.

In some embodiments, the compound can be represented by Formula (Ia), (Ib), (Ic), (Id), (Ie), (If), (Ig), (Ih) or (Ii), or a pharmaceutically acceptable salt thereof, wherein R^{4B} and R^{4C} can each independently be:
(A) —OH;
(B) —OR^{4B1}, wherein
   R^{4B1} can be C₁₋₆ alkyl optionally substituted with 1 to 3 R^{4B2} groups, C₁₋₆ haloalkyl, C₃₋₈ cycloalkyl, C₆₋₁₂ aryl, or a 5 to 6 membered heteroaryl having 1 to 3 heteroatoms each independently selected from N, O or S, wherein
      each R^{4B2} group can independently be C₁₋₆ alkoxy, —S—R^{4B3}, or —S(O)₂—R^{4B3}, and
      each R^{4B3} group can independently be C₁₋₆ alkyl;

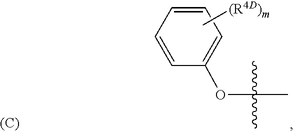

wherein
   subscript m can be 0, 1, 2, 3, 4, or 5; and
   each R^{4D} can independently be C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein
      each R^{4D1} group can independently be —NH₂ or —C(O)OR^{4D3},
      each R^{4D2} can independently be C₁₋₃ alkoxy, and
      each R^{4D3} can independently be C₁₋₃ alkyl;

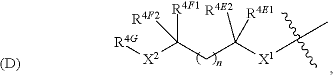

wherein
   X¹ and X² can each independently be —O— or —N(R^{4H})—;
   R^{4E1} and R^{4E2} can each independently be H, C₁₋₆ alkyl optionally substituted with 1 to 3 R^{4E3} groups, or C₃₋₆ cycloalkyl, wherein
      each R^{4E3} group can independently be —C(O)OR^{4E4}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and
      each R^{4E4} group can independently be C₁₋₆ alkyl;
   or R^{4E1} and R^{4E2} are combined with the atom to which they are attached to form a C₃₋₆ cycloalkyl;
   R^{4F1} and R^{4F2} are each H or together are oxo;
   R^{4G} can be C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

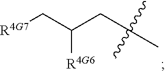

each R^{4G1} can independently be —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl;
each R^{4G2} can independently be C₁₋₆ alkyl, C₁₋₆ alkoxy, halogen, C₁₋₃ haloalkyl, —OH, —NH₂, or C₆₋₁₂ aryl;
each R^{4G3} can independently be C₁₋₆ alkyl, halogen, C₁₋₃ haloalkyl, oxo, —C(O)R^{4G5}, or —C(O)OR^{4G5};
each R^{4G4} can independently be C₁₋₆ alkyl, C₇₋₁₈ alkyl or C₃₋₈ cycloalkyl, wherein the C₁₋₆ alkyl can be optionally substituted with OH, NH₂, or —NHC(O)OR^{4G5}, and wherein the cycloalkyl can be optionally substituted with C₁₋₆ alkyl;
each R^{4G5} can independently be C₁₋₆ alkyl;
R^{4G6} and R^{4G7} can each independently be H or —OR^{4G11}, wherein at least one of R^{4G6} and R^{4G7} can be —OR^{4G11};
each R^{4G8} can independently be H or C₁₋₆ alkyl;
each R^{4G9} can independently be C₁₋₆ alkyl, halogen, C₁₋₃ haloalkyl, or —NH₂;
each R^{4G10} can independently be C₁₋₆ alkyl, C₁₋₃ haloalkyl, or oxo;
each R^{4G11} can independently be C₁₀₋₁₈ alkyl or benzyl;
R^{4H} can be H;
or R^{4E1} and R^{4H} are combined with the atoms to which they are attached to form a 5 to 6 membered heterocyclyl having 1 to 2 additional heteroatoms selected from N, O and S; and
subscript n can be 0 or 1; or
(E) —(OP(O)(OH))₁₋₂—OH; or

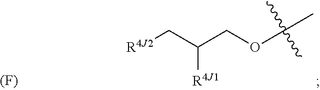

wherein
   R^{4J1} and R^{4J2} can each independently be H, —OR^{4J3} or —OC(O)R^{4J3}, wherein
      at least one of R^{4J1} and R^{4J2} can be —OR^{4J3} or —OC(O)R^{4J3},
      each R^{4J3} can independently be C₁₋₁₈ alkyl, C₂₋₆ alkenyl, or benzyl, and at least one R^{4J3} can be C₁₀₋₁₈ alkyl;

In some embodiments, the compound can be represented by Formula (Ia), (Ib), (Ic), (Id), (Ie), (If), (Ig), (Ih) or (Ii), or a pharmaceutically acceptable salt thereof, wherein R² and R^{4C} can be combined with the atoms to which they are attached to form a six-membered ring. In some embodiments, the compound of Formula (Ia) can have the following structure:

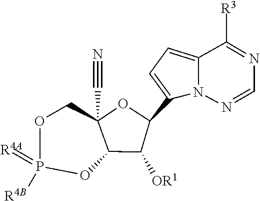

or a pharmaceutically acceptable salt thereof.

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be —OH.

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be —OR^{4B1}, wherein R^{4B1} is C₁₋₆ alkyl optionally substituted with 1 to 3 R^{4B2} groups, C₁₋₆ haloalkyl, C₃₋₈ cycloalkyl, C₆₋₁₂ aryl, or a 5 to 6 membered heteroaryl having 1 to 3 heteroatoms each independently selected from N, O or S, wherein each R^{4B2} group is independently C₁₋₆ alkoxy, —S—R^{4B3}, or —S(O)₂—R^{4B3}, and each R^{4B3} group is independently C₁₋₆ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4B1} can be methyl, ethyl, n-propyl, iso-propyl, —CH₂F, —CHF₂, —CF₃, 2,2,2-trifluoroethyl, methoxymethyl, ethoxymethyl, 2-methoxyethyl, 2-ethoxyethyl, 2-(thiomethyl)ethyl, 2-(methylsulfonyl)ethyl, cyclopropyl, cyclobutyl, cyclopentyl, cyclohexyl, naphthyl, or pyridyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4B1} can be iso-propyl, 2,2,2-trifluoroethyl, 2-methoxyethyl, 2-(thiomethyl)ethyl, 2-(methylsulfonyl)ethyl, cyclopentyl, naphthyl, or pyridyl.

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be

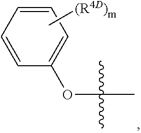

wherein subscript m is 0, 1, 2, 3, 4, or 5; and each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein each R^{4D1} group is independently —NH₂ or —C(O)OR^{4D3}, each R^{4D2} is independently C₁₋₃ alkoxy, and each R^{4D3} is independently C₁₋₃ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4D} can be methyl, ethyl, n-propyl, iso-propyl, n-butyl, iso-butyl, sec-butyl, tert-butyl, methoxymethyl, 2-methoxyethoxy, —C(O)OMe, —C(O)OEt, —C(O)NMe₂, or

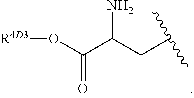

In some embodiments, the compound can be represented by Formula (Ia), (Ib), (Ic), (Id), (Ie), (If), (Ig), (Ih) or (Ii), or a pharmaceutically acceptable salt thereof, wherein R^{4D} can be tert-butyl, 2-methoxyethoxy, —C(O)OEt, —C(O)NMe₂, or

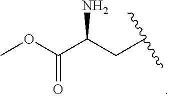 [SMILES: *C[C@H](N)C(=O)OC]

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be

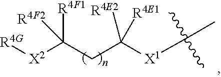

wherein X¹ and X² are each independently —O— or —N(R^{4H})—;
R^{4E1} and R^{4E2} are each independently H, C₁₋₆ alkyl optionally substituted with 1 to 3 R^{4E3} groups, or C₃₋₆ cycloalkyl, wherein each R^{4E3} group is independently-C(O)OR^{4E3}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and each R^{4F4} group is independently C₁₋₆ alkyl; or R^{4E1} and R^{4E2} are combined with the atom to which they are attached to form a C₃₋₆ cycloalkyl;
R^{4F1} and R^{4F2} are each H or together are oxo;
R^{4G} is C₁₋₆ alkyl optionally substituted with 1 to 3 R^{4G1} C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

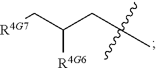

each R^{4G1} is independently —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl; each R^{4G2} is independently C₁₋₆ alkyl, C₁₋₆ alkoxy, halogen, C₁₋₃ haloalkyl, —OH, —NH₂, or C₆₋₁₂ aryl; each R^{4G3} is independently C₁₋₆ alkyl, halogen, C₁₋₃ haloalkyl, oxo, —C(O)R^{4G5}, or —C(O)OR^{4G5};
each R^{4G4} is independently C₁₋₆ alkyl, C₇₋₁₈ alkyl or C₃₋₈ cycloalkyl, wherein the C₁₋₆ alkyl is optionally substituted with OH, NH₂, or —NHC(O)OR^{4G5}, and wherein the cycloalkyl is optionally substituted with C₁₋₆ alkyl;
each R^{4G5} is independently C₁₋₆ alkyl;
R^{4G6} and R^{4G7} are each independently H or —OR^{4G11}, wherein at least one of R^{4G6} and R^{4G7} is —OR^{4G11};
each R^{4G8} is independently H or C₁₋₆ alkyl;
each R^{4G9} is independently C₁₋₆ alkyl, halogen, C₁₋₃ haloalkyl, or —NH₂; each R^{4G10} is independently C₁₋₆ alkyl, C₁₋₃ haloalkyl, or oxo; each R^{4G11} is independently C₁₀₋₁₈ alkyl or benzyl;
R^{4H} is H; or R^{4E1} and R^{4H} are combined with the atoms to which they are attached to form a 5 to 6 membered heterocyclyl having 1 to 2 additional heteroatoms selected from N, O and S; and subscript n is 0 or 1.

In some embodiments, the compound can be represented by Formula (Ia), (Ib), (Ic), (Id), (Ie), (If), (Ig), (Ih) or (Ii), or a pharmaceutically acceptable salt thereof, wherein X¹ and X² are each independently —O— or —NH—. In some embodiments, the compound can be represented by Formula (Ia), (Ib), (Ic), (Id), (Ie), (If), (Ig), (Ih) or (Ii), or a pharmaceutically acceptable salt thereof, wherein X¹ and X² are each —O—. In some embodiments, the compound can be represented by Formula (Ia), (Ib), (Ic), (Id), (Ie), (If), (Ig), (Ih) or (Ii), or a pharmaceutically acceptable salt thereof, wherein X¹ and X² are each —NH—. In some embodiments, the compound can be represented by Formula (Ia), (Ib), (Ic), (Id), (Ie), (If), (Ig), (Ih) or (Ii), or a pharmaceutically acceptable salt thereof, wherein X¹ can be —O— and X² can be —NH—. In some embodiments, the compound can be represented by Formula (Ia), (Ib), (Ic), (Id), (Ie), (If), (Ig), (Ih) or (Ii), or a pharmaceutically acceptable salt thereof, wherein X¹ can be —NH— and X² can be —O—.

In some embodiments, the compound can be represented by Formula (Ia), (Ib), (Ic), (Id), (Ie), (If), (Ig), (Ih) or (Ii), or a pharmaceutically acceptable salt thereof, wherein R^{4F1} and R^{4F2} are each H. In some embodiments, the compound can be represented by Formula (Ia), (Ib), (Ic), (Id), (Ie), (If), (Ig), (Ih) or (Ii), or a pharmaceutically acceptable salt thereof, wherein R^{4F1} and R^{4F2} together are oxo.

In some embodiments, the compound can be represented by Formula (Ia), (Ib), (Ic), (Id), (Ie), (If), (Ig), (Ih) or (Ii), or a pharmaceutically acceptable salt thereof, wherein subscript n is 0. In some embodiments, the compound can be represented by Formula (Ia), (Ib), (Ic), (Id), (Ie), (If), (Ig), (Ih) or (Ii), or a pharmaceutically acceptable salt thereof, wherein subscript n is 1.

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be

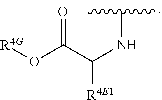

In some embodiments, the compound can be represented by Formula (Ia), (Ib), (Ic), (Id), (Ie), (If), (Ig), (Ih) or (Ii), or a pharmaceutically acceptable salt thereof, wherein R^{4E1} can be H, C₁₋₆ alkyl optionally substituted with 1 to 3 R^{4E3} groups, or C₃₋₆ cycloalkyl, wherein each R^{4E3} group is independently-C(O)OR^{4E4}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and each R^{4E4} group is independently C₁₋₆ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4E1} can be C₁₋₆ alkyl optionally substituted with 1 R^{4E3} group, cyclopropyl, cyclobutyl, cyclopentyl, or cyclohexyl, wherein each R^{4E3} group is independently-C(O)OR^{4E4}, —NH₂, —NHC(O)R^{4E4}, —NHC(O)O— benzyl, cyclopropyl, cyclobutyl, cyclopentyl, cyclohexyl, or phenyl, and each R^{4E4} group is independently methyl, ethyl, iso-propyl, n-butyl, or iso-butyl. In some embodiments, the compound can be represented by Formula (Ia), (Ib), (Ic), (Id), (Ie), (If), (Ig), (Ih) or (Ii), or a pharmaceutically acceptable salt thereof, wherein R^{E1} can be methyl, ethyl, iso-propyl, n-butyl, iso-butyl, cyclopropylmethyl, cyclopentyl, cyclohexyl, or benzyl, wherein the methyl, ethyl and butyl are each optionally substituted with —NH₂, —NHC(O)Me, —NHC(O)O-benzyl, —C(O)O— butyl, —C(O)O-pentyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4E1} can be methyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be C₁₋₆ alkyl optionally substituted with 1 to 3 R^{4G1}, wherein each R^{4G1} is independently —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl; each R^{4G8} is independently H or C₁₋₆ alkyl; each R^{4G9} is independently C₁₋₆ alkyl, halogen, C₁₋₃ haloalkyl, or —NH₂; and each R^{4G10} is independently C₁₋₆ alkyl, C₁₋₃ haloalkyl, or oxo.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be
methyl optionally substituted with —(CH₂OCH₂)₂—CH₃, cyclopropyl, cyclobutyl, cyclohexyl optionally substituted with CF₃ or NH₂, piperidine optionally substituted with CH₂CF₃, quinuclidine, oxetane, tetrahydro-2H-pyran, or phenyl,
ethyl optionally substituted with NMe₂ or N(iPr)₂,
n-propyl optionally substituted with methoxy or morpholine,
iso-propyl optionally substituted with C(O)NH₂,
n-butyl,
iso-butyl optionally substituted with methoxy, OH or CF₃,
pentyl, neopentyl, hexyl, 2,2-dimethylbutyl, 3,3-dimethylbutyl, or 2-ethyl-butyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be methyl, ethyl, n-propyl, iso-propyl, n-butyl, pentyl, neopentyl, hexyl, 2,2-dimethylbutyl, 3,3-dimethylbutyl, 2-ethyl-butyl,

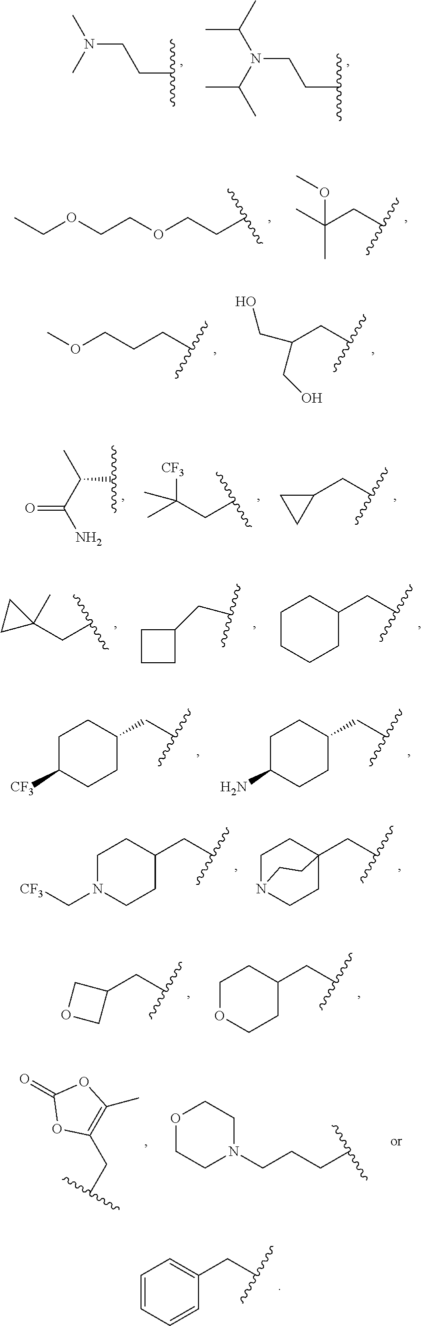

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be C₇₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be heptyl, octyl, nonyl, decyl, undecyl, docecyl, hexadecyl or octadecyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be octyl, dodecyl, hexadecyl or octadecyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be C₃₋₈ cycloalkyl optionally substituted with 1 to 3 R^{4G2}, wherein each R^{4G2} is independently C₁₋₆ alkyl, C₁₋₆ alkoxy, halogen, C₁₋₃ haloalkyl, —OH, —NH₂, or C₆₋₁₂ aryl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be C₃₋₈ cycloalkyl optionally substituted with 1 to 3 R^{4G2}, wherein each R^{4G2} is independently methyl, ethyl, n-propyl, iso-propyl, n-butyl, iso-butyl, sec-butyl, tert-butyl, methoxy, ethoxy, propoxy, iso-propoxy, F, Cl, —CH₂F, —CHF₂, —CF₃, —CH₂CF₃, —NH₂ or phenyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be cyclopropyl, cyclobutyl, cyclopentyl, cyclohexyl, cycloheptyl or cyclooctyl, each optionally substituted with 1 to 2 R^{4G2}, wherein each R^{4G2} is independently methyl, tert-butyl, methoxy, F, —CF₃, —NH₂ or phenyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be cyclobutyl, cyclopentyl, cyclohexyl, cycloheptyl or cyclooctyl, wherein the cyclohexyl can be optionally substituted with 1 to 2 R^{4G2}, wherein each R^{4G2} is independently methyl, tert-butyl, methoxy, F, —CF₃, —NH₂ or phenyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be cyclobutyl, cyclopentyl, cyclohexyl, cycloheptyl, cyclooctyl,

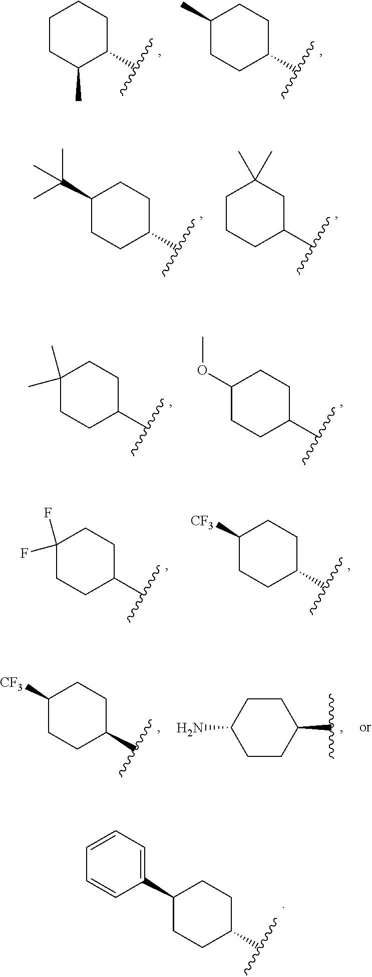

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be a 3 to 8 membered heterocyclyl having 1 to 3 heteroatoms selected from N, O and S, optionally substituted with 1 to 3 R^{4G3}, wherein each R^{4G3} is independently C₁₋₆ alkyl, halogen, C₁₋₃ haloalkyl, oxo, —C(O)R^{4G5}, or —C(O)OR^{4G5}.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be pyrrolidine, piperidine, azepane, quinuclidine, oxetane, tetrahydrofuran, tetrahydropyran, piperazine or morpholine, each optionally substituted with 1 to 3 R^{4G3}, wherein each R^{4G3} is independently C₁₋₆ alkyl, halogen, C₁₋₃ haloalkyl, oxo, —C(O)R^{4G5}, or —C(O)OR^{4G5}. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be pyrrolidine, piperidine, azepane, quinuclidine, oxetane, tetrahydrofuran, tetrahydropyran, piperazine or morpholine, each optionally substituted with 1 to 3 R^{4G3}, wherein each R^{4G3} is independently methyl, ethyl, n-propyl, iso-propyl, n-butyl, iso-butyl, sec-butyl, tert-butyl, F, Cl, —CH₂F, —CHF₂, —CF₃, —CH₂CF₃, oxo, —C(O)Me, or —C(O)O—C₁₋₄ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be pyrrolidine, piperidine, azepane, quinuclidine, oxetane, tetrahydrofuran, tetrahydropyran or morpholine, each optionally substituted with 1 to 3 R^{4G3}, wherein each R^{4G3} is independently methyl, ethyl, F, —CH₂CF₃, oxo, —C(O)Me, or —C(O)O-tert-butyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be pyrrolidine optionally substituted with methyl, oxo, —C(O)Me, or —C(O)O-tert-butyl, piperidine optionally substituted with methyl, ethyl, F, or —C(O)Me, azepane, quinuclidine, oxetane, tetrahydrofuran optionally substituted with methyl, tetrahydropyran optionally substituted with methyl, or morpholine. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be:

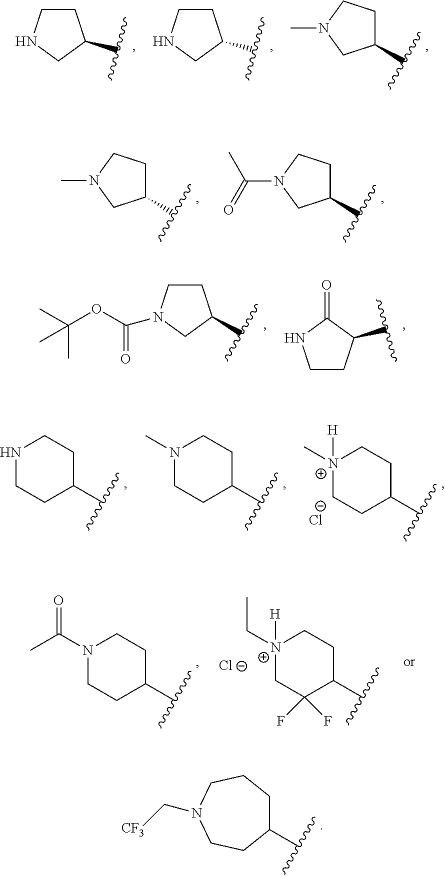

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be —C(O)R^{4G4}, wherein each R^{4G4} is independently C₁₋₆ alkyl, C₇₋₁₈ alkyl or C₃₋₈ cycloalkyl, wherein the C₁₋₆ alkyl is optionally substituted with OH, NH₂, or —NHC(O)OR^{4G5}, and wherein the cycloalkyl is optionally substituted with C₁₋₆ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be —C(O)R^{4G4}, wherein each R^{4G4} is independently methyl, ethyl optionally substituted with NH₂, n-propyl, iso-propyl, n-butyl, iso-butyl optionally substituted with NH₂ or —NHC(O)O-tert-butyl, sec-butyl, tert-butyl optionally substituted with OH, pentyl, neopentyl, hexyl, heptyl, octyl, nonyl, decyl, undecyl, dodecyl, hexadecyl, octadecyl, cyclopropyl optionally substituted with methyl, cyclobutyl, cyclopentyl, or cyclohexyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be —C(O)R^{4G4}, wherein each R^{4G4} is independently ethyl optionally substituted with NH₂, iso-butyl optionally substituted with NH₂ or —NHC(O)O-tert-butyl, tert-butyl optionally substituted with OH, undecyl, cyclopropyl optionally substituted with methyl, or cyclohexyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be

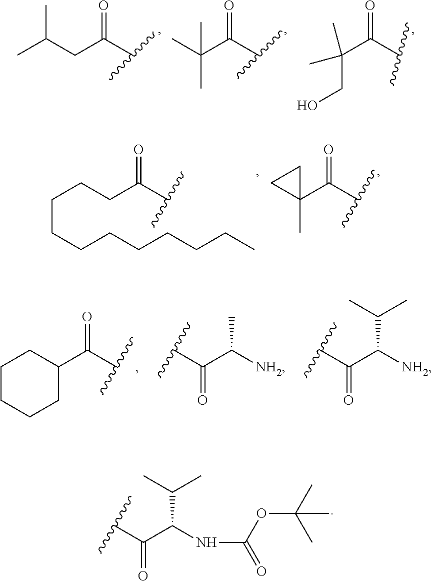

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be —C(O)OR^{4G5}, wherein each R^{4G5} is independently C₁₋₆ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G5} can be methyl, ethyl, n-propyl, iso-propyl, n-butyl, iso-butyl, sec-butyl or tert-butyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G5} can be iso-butyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be:

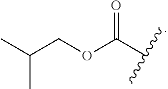 [SMILES: *C(=O)OCC(C)C]

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be

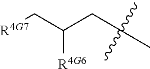

wherein R^{4G6} and R^{4G7} are each independently H or —OR^{4G11}, wherein at least one of R^{4G6} and R^{4G7} is —OR^{4G11}; and each R^{4G11} is independently C₁₀₋₁₈ alkyl or benzyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G6} can be H and R^{4G7} can be —OR^{4G11}, wherein R^{4G11} can be C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G6} and R^{4G7} are each independently-OR^{4G11}, wherein each R^{4G11} is independently C₁₀₋₁₈ alkyl or benzyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} can be:

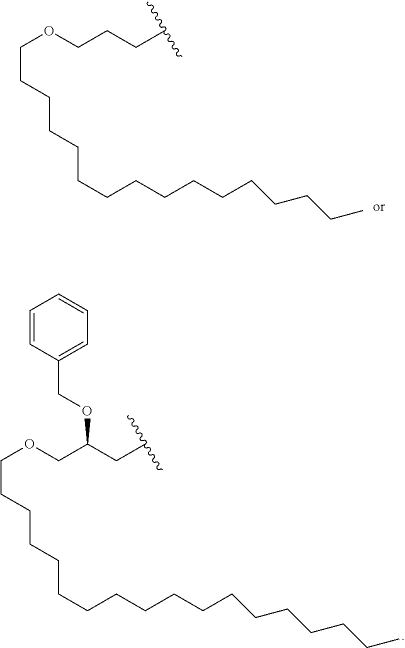 [SMILES: *CCCOCCCCCCCCCCCCCCCC ; *C[C@@H](COCCCCCCCCCCCCCCCCCC)OCc1ccccc1]

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be —(OP(O)(OH))₁₋₂—OH.

In some embodiments, the compound can be represented by Formula (Ia), (Ib), (Ic), (Id), (Ie), (If), (Ig), (Ih) or (Ii), or a pharmaceutically acceptable salt thereof, wherein one of R^{4B} and R^{4C} can be

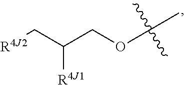

wherein R^{4J1} and R^{4J2} are each independently H, —OR^{4J3} or —OC(O)R^{4J3}, wherein at least one of R^{4J1} and R^{4J2} is —OR^{4J3} or —OC(O)R^{4J3}, each R^{4J3} is independently C₁₋₁₈ alkyl, C₂₋₆ alkenyl, or benzyl, and at least one R^{4J3} is C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} can be H and R^{4J2} can be —OR^{4J3} or —OC(O)R^{4J3}, wherein each R^{4J3} is independently C₁₋₁₈ alkyl, C₂₋₆ alkenyl, or benzyl, and at least one R^{4J3} is C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} and R^{4J2} are each independently —OR^{4J3} or —OC(O)R^{4J3}, wherein each R^{4J3} is independently C₁₋₁₈ alkyl, C₂₋₆ alkenyl, or benzyl, and at least one R^{4J3} is C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} can be —OR^{4J3}, wherein R^{4J3} can be C₁₋₆ alkyl, C₂₋₆ alkenyl, or benzyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} can be —OR^{4J3}, wherein R^{4J3} can be methyl, ethyl, n-propyl, iso-propyl, prop-2-enyl, but-2-enyl, but-3-enyl, or benzyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} can be —OR^{4J3}, wherein R^{4J3} can be methyl, prop-2-enyl, or benzyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J2} can be —OR^{4J3}, wherein R^{4J3} can be C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J2} can be —OR^{4J3}, wherein R^{4J3} can be dodecyl, undecyl, dodecyl, hexadecyl or octadecyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J2} can be —OR^{4J3}, wherein R^{4J3} can be hexadecyl or octadecyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} and R^{4J2} are each —OC(O)R^{4J3}, wherein R^{4J3} is C₁₀₋₁₈ alkyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} and R^{4J2} are each —OC(O)R^{4J3}, wherein R^{4J3} can be dodecyl, undecyl, dodecyl, pentadecyl, hexadecyl or octadecyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4J1} and R^{4J2} are each —OC(O)R^{4J3}, wherein R^{4J3} can be pentadecyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4B} and R^{4C} are each independently:

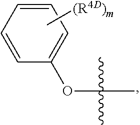

wherein
subscript m is 0, 1, 2, 3, 4, or 5; and each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein each R^{4D1} group is independently —NH₂ or —C(O)OMe, each R^{4D2} is methoxy, and each R^{4D3} is independently methyl or ethyl; or

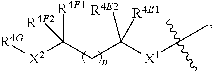

wherein
X¹ and X² are each independently —O— or —NH—;
R^{4E1} is C₁₋₆ alkyl optionally substituted with 1 R^{4E3} group, or C₃₋₆ cycloalkyl, wherein each R^{4E3} group is independently —C(O)Me, —C(O)O-n-butyl, —C(O)O-pentyl, —NH₂, —NHC(O)Me, —NHC(O)O-benzyl, C₃₋₆ cycloalkyl or phenyl;
R^{4E2} is H; or R^{4E1} and R^{4E2} are combined with the atom to which they are attached to form a C₃₋₆ cycloalkyl;
R^{4F1} and R^{4F2} are each H or together are oxo;
R^{4G} is C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

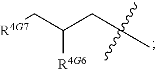

each R^{4G1} is independently —OH, hydroxymethyl, methoxy, —(CH₂OCH₂)₂—CH₃, —CF₃, —N(Me)₂, —C(O)NH₂, C₃₋₈ cycloalkyl optionally substituted with 1 to 2 R^{4G9}, a 3 to 8 membered heterocyclyl having 1 to 3 heteroatoms selected from N, O and S, optionally substituted with 1 to 2 R^{4G10}, or phenyl;
each R^{4G2} is independently methyl, ethyl, n-propyl, iso-propyl, n-butyl, iso-butyl, sec-butyl, t-butyl, methoxy, F, Cl, Br, CF₃, —NH₂, or phenyl;
each R^{4G3} is independently methyl, ethyl, F, Cl, CF₃, CH₂CF₃, oxo, —C(O)Me, or —C(O)O-t-butyl;
   each R^{4G4} is independently C₁₋₆ alkyl, C₇₋₁₈ alkyl or C₃₋₇ cycloalkyl, wherein the C₁₋₆ alkyl is optionally substituted with OH, NH₂, or —NHC(O)O-t-butyl, and wherein the cycloalkyl is optionally substituted with methyl, ethyl, n-propyl, iso-propyl, n-butyl, iso-butyl, sec-butyl, or t-butyl;
each R^{4G5} is independently methyl, ethyl, n-propyl, iso-propyl, n-butyl, iso-butyl, sec-butyl, or t-butyl;
R^{4G6} and R^{4G7} are each independently H or —OR^{4G11}, wherein at least one of R^{4G6} and R^{4G7} is —OR^{4G11};
each R^{4G9} is independently methyl, CF₃, or —NH₂;
each R^{4G10} is independently methyl, CF₃, CH₂CF₃, or oxo; and
each R^{4G11} is independently hexadecane, octadecane or benzyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4C} can be:

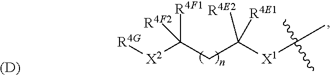

wherein
X¹ and X² are each independently —O— or —NH—;

R^{4E1} is C₁₋₆ alkyl optionally substituted with 1 R^{4E3} group, or C₃₋₆ cycloalkyl, wherein each R^{4E3} group is independently —C(O)Me, —C(O)O-n-butyl, —C(O)O-pentyl, —NH₂, —NHC(O)Me, —NHC(O)O-benzyl, C₃₋₆ cycloalkyl or phenyl;
R^{4E2} is H; or R^{4E1} and R^{4E2} are combined with the atom to which they are attached to form a C₃₋₆ cycloalkyl;
R^{4F1} and R^{4F2} are each H or together are oxo;
R^{4G} is C₁₋₆ alkyl optionally substituted with 1 to 3 R^{4G1} C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or

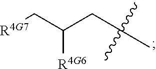

each R^{4G1} is independently —OH, hydroxymethyl, methoxy, —(CH₂OCH₂)₂—CH₃, —CF₃, —N(Me)₂, —C(O)NH₂, C₃₋₈ cycloalkyl optionally substituted with 1 to 2 R^{4G9}, a 3 to 8 membered heterocyclyl having 1 to 3 heteroatoms selected from N, O and S, optionally substituted with 1 to 2 R^{4G10}, or phenyl;
each R^{4G2} is independently methyl, ethyl, n-propyl, iso-propyl, n-butyl, iso-butyl, sec-butyl, t-butyl, methoxy, F, Cl, Br, CF₃, —NH₂, or phenyl;
each R^{4G3} is independently methyl, ethyl, F, Cl, CF₃, CH₂CF₃, oxo, —C(O)Me, or —C(O)O-t-butyl;
each R^{4G4} is independently C₁₋₆ alkyl, C₇₋₁₈ alkyl or C₃₋₇ cycloalkyl, wherein the C₁₋₆ alkyl is optionally substituted with OH, NH₂, or —NHC(O)O-t-butyl, and wherein the cycloalkyl is optionally substituted with methyl, ethyl, n-propyl, iso-propyl, n-butyl, iso-butyl, sec-butyl, or t-butyl;
each R^{4G5} is independently methyl, ethyl, n-propyl, iso-propyl, n-butyl, iso-butyl, sec-butyl, or t-butyl;
R^{4G6} and R^{4G7} are each independently H or —OR^{4G11}, wherein at least one of R^{4G6} and R^{4G7} is —OR^{4G11};
each R^{4G9} is independently methyl, CF₃, or —NH₂;
each R^{4G10} is independently methyl, CF₃, CH₂CF₃, or oxo; and
each R^{4G11} is independently hexadecane, octadecane or benzyl.

In some embodiments, the compound is of Formula (Ib):

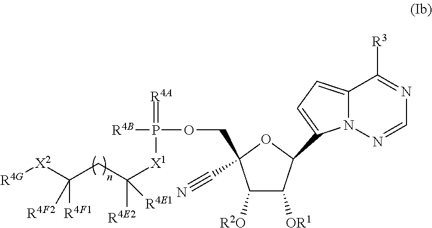

or a pharmaceutically acceptable salt thereof.

In some embodiments, the compound is of Formula Ic:

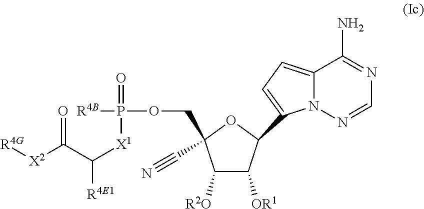

or a pharmaceutically acceptable salt thereof.

In some embodiments, the compound can be represented by Formula (Ia), (Ib), (Ic), (Id), (Ie), (If), (Ig), (Ih) or (Ii), or a pharmaceutically acceptable salt thereof, wherein R^{4B} can be:

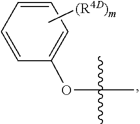

wherein subscript m is 1; and
R^{4D} is independently methyl, ethyl, n-propyl, or tert-butyl, each optionally substituted with 1 to 3 R^{4D1} groups, wherein each R^{4D1} group is independently —NH₂ or —C(O)OMe, or
R^{4D} is methoxy, ethoxy, or propoxy, each optionally substituted with methoxy, or
R^{4D} is —C(O)OMe, —C(O)OEt or —C(O)N(Me)₂; and
R^{4C} can be:

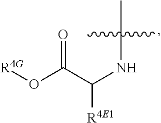

wherein R^{4E1} is methyl, ethyl, n-propyl, iso-propyl, n-butyl, iso-butyl, sec-butyl, t-butyl, n-pentane, neopentane, or n-hexane, each optionally substituted with 1 R^{4E3} group wherein each R^{4E3} group is independently —C(O)Me, —C(O)O-n-butyl, —C(O)O— pentyl, —NH₂, —NHC(O)Me, or —NHC(O)O-benzyl, or
R^{4E1} is cyclopropyl, cyclopropylmethyl, cyclobutyl, cyclobutylmethyl, cyclopentyl, cyclopentylmethyl, cyclohexyl, or cyclohexylmethyl, or
R^{4E1} is benzyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4B} can be

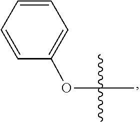 [SMILES: *Oc1ccccc1]

and

### R^{4C} can be

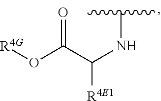 [0291] wherein R^{E1} can be methyl, ethyl, iso-propyl, n-butyl, iso-butyl, cyclopropylmethyl, cyclopentyl, cyclohexyl, or benzyl, wherein the methyl, ethyl and butyl groups are each optionally substituted with —NH₂, —NHC(O)Me, —NHC(O)O-benzyl, —C(O)O-butyl, —C(O)O-pentyl; and
R^{4G} is methyl, ethyl, n-propyl, iso-propyl, n-butyl, pentyl, neopentyl, hexyl, 2,2-dimethylbutyl, 3,3-dimethylbutyl, 2-ethyl-butyl, octyl, dodecyl, hexadecyl, octadecyl, cyclobutyl, cyclopentyl, cyclohexyl, cycloheptyl, cyclooctyl,

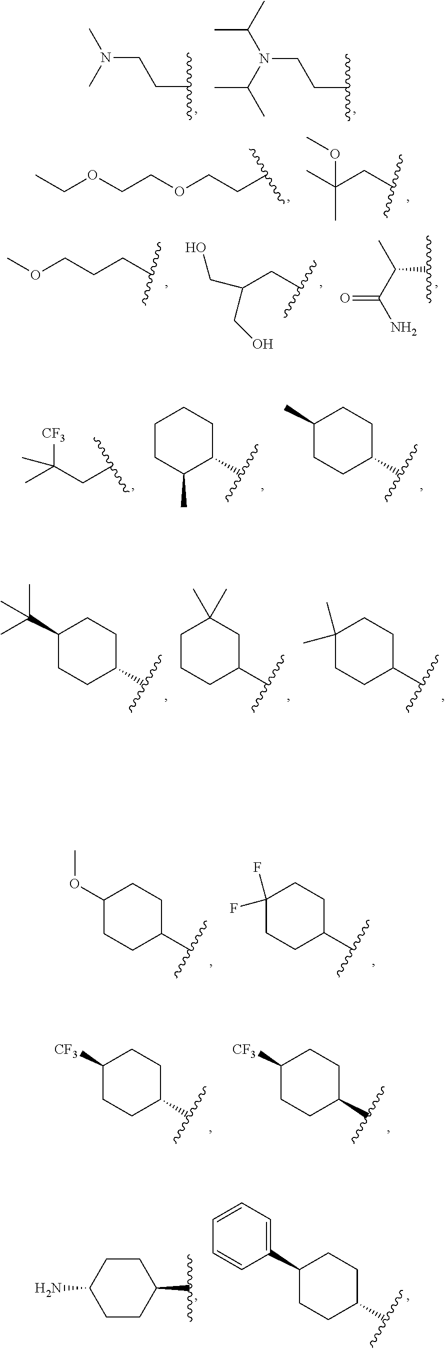

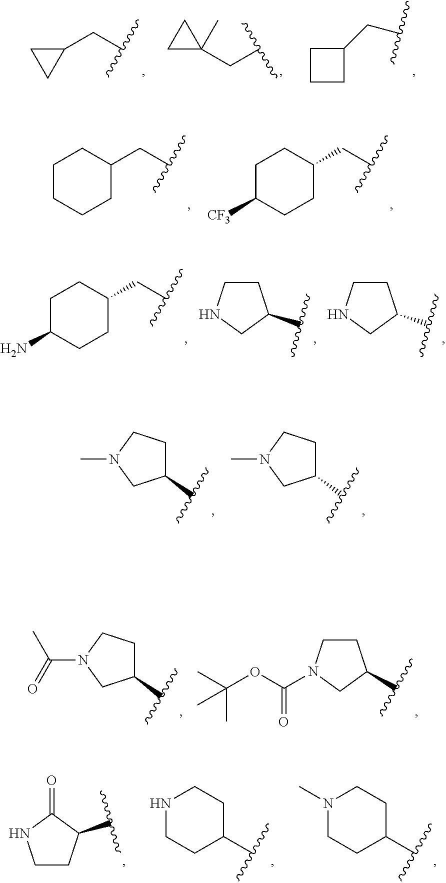

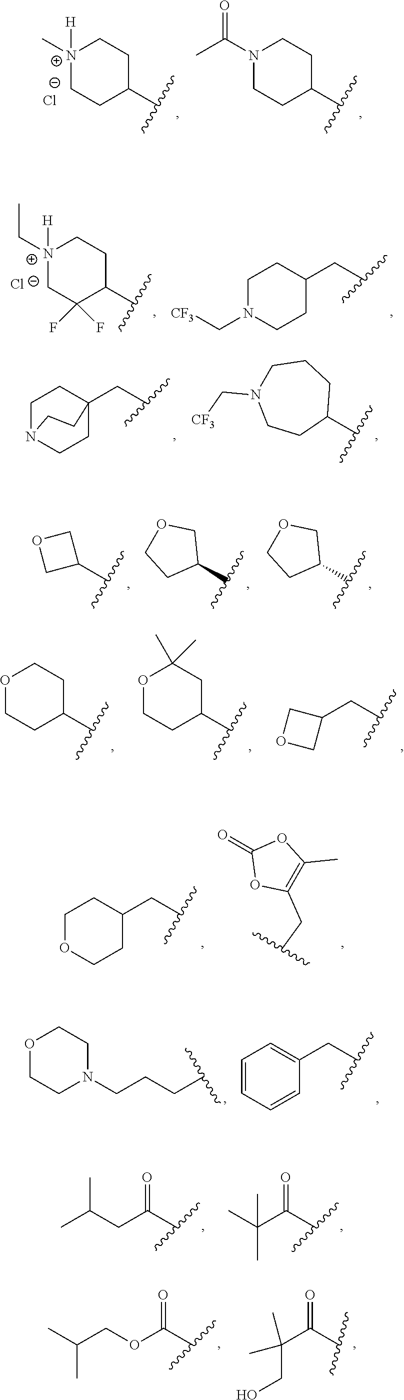

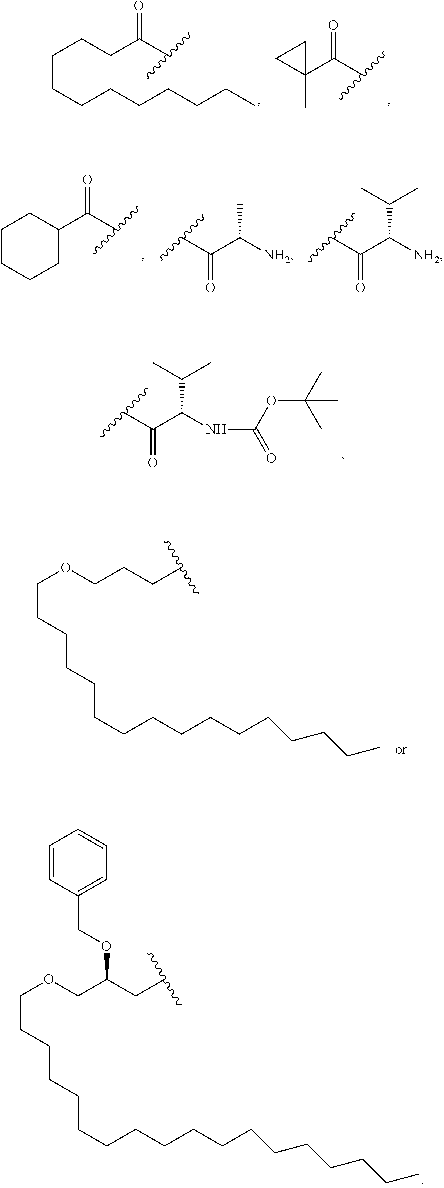

In some embodiments, the compound is of Formula (Id):

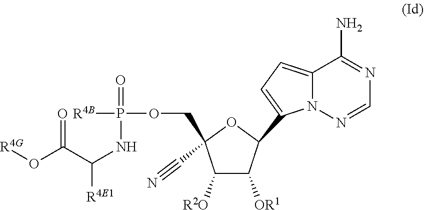

or a pharmaceutically acceptable salt thereof.

In some embodiments, the compound is of Formula (Ie):

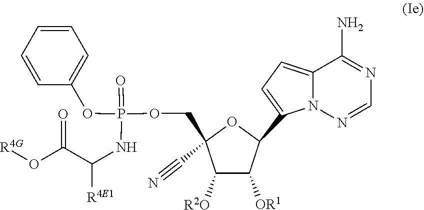

or a pharmaceutically acceptable salt thereof.

In some embodiments, the compound is of Formula (If):

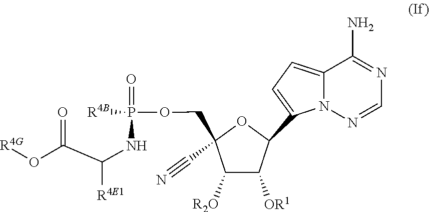

or a pharmaceutically acceptable salt thereof.

In some embodiments, the compound is of Formula (Ig):

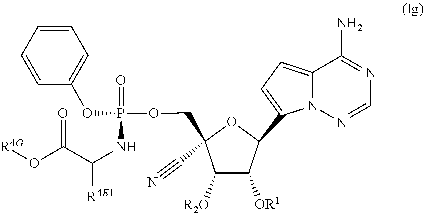

or a pharmaceutically acceptable salt thereof.

In some embodiments, the compound is of Formula (Ih):

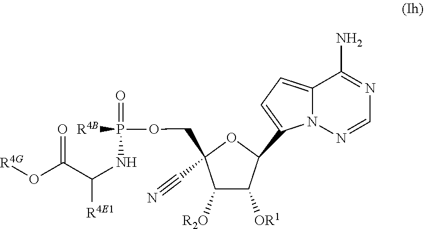

or a pharmaceutically acceptable salt thereof.

In some embodiments, the compound is of Formula (Ii):

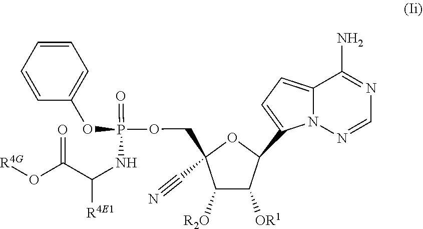

or a pharmaceutically acceptable salt thereof.

In some embodiments, the compound can be represented by Formula (Ia), (Ib), (Ic), (Id), (Ie), (If), (Ig), (Ih) or (Ii), or a pharmaceutically acceptable salt thereof, wherein the compound, or a pharmaceutically acceptable salt thereof, is the compound wherein R^{4C} is:

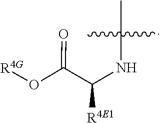

In some embodiments, the compound can be represented by Formula (Ia), (Ib), (Ic), (Id), (Ie), (If), (Ig), (Ih) or (Ii), or a pharmaceutically acceptable salt thereof, wherein the compound, or a pharmaceutically acceptable salt thereof, is the compound wherein R^{4C} is:

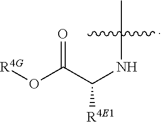

In some embodiments, the compound can be represented by Formula (Ia), (Ib), (Ic), (Id), (le), (If), (Ig), (Ih) or (Ii), or a pharmaceutically acceptable salt thereof, wherein R^{4G} is methyl, ethyl, n-propyl, iso-propyl, n-butyl, iso-butyl, sec-butyl, t-butyl, n-pentane, neopentane, n-hexane, 2,2-dimethylbutyl, 3,3-dimethylbutyl, 2-ethyl-butyl, heptane, octane, nonane, decane, undecane, dodecane, pentadecane, hexadecane, or octadecane, each optionally substituted with 1 to 2 R^{4G1} wherein each R^{4G1} is independently —OH, hydroxymethyl, methoxy, —(CH₂OCH₂)₂—CH₃, —CF₃, —N(Me)₂, or —C(O)NH₂.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is cyclopropyl, cyclobutyl, cyclopentyl, cyclohexyl, cycloheptyl, or cyclooctyl, each optionally substituted with 1 to 2 R^{4G2} wherein each R^{4G2} is independently methyl, ethyl, n-propyl, iso-propyl, n-butyl, iso-butyl, sec-butyl, t-butyl, OMe, F, CF₃, —NH₂, or phenyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is cyclopropylmethyl, cyclobutylmethyl, cyclopentylmethyl, cyclohexylmethyl, cycloheptylmethyl, or cyclooctylmethyl, each optionally substituted with 1 to 2 R^{4G2} wherein each R^{4G2} is independently methyl, CF₃, or —NH₂.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is pyrrolidine, piperidine, azepane, quinuclidine, oxetane, tetrahydrofuran, tetrahydropyran, morpholine, or 1,3-dioxol, each optionally substituted with 1 to 2 R^{4G3} wherein each R^{4G3} is independently methyl, ethyl, F, CH₂CF₃, oxo, —C(O)Me, or —C(O)O-t-butyl. In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is piperidinemethyl, quinuclidinemethyl, oxetanemethyl, tetrahydrofuranmethyl, tetrahydropyranmethyl, morpholinemethyl, 2-morpholine-ethyl, 3-morpholine-propyl, or 1,3-dioxolmethyl, each optionally substituted with 1 to 2 R^{4G10} wherein each R^{4G10} is independently methyl, CH₂CF₃, or oxo.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is benzyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is —C(O)R^{4G4}, wherein R^{4G4} is C₁₋₆ alkyl selected from the group consisting of methyl, ethyl, n-propyl, iso-propyl, n-butyl, iso-butyl, sec-butyl, t-butyl, n-pentane, neopentane, n-hexane, 2,2-dimethylbutyl, 3,3-dimethylbutyl, and 2-ethyl-butyl, a C₇₋₁₈ alkyl selected from the group consisting of heptane, octane, nonane, decane, undecane, dodecane, pentadecane, hexadecane, and octadecane, or C₃₋₈ cycloalkyl selected from the group consisting of cyclopropyl, cyclobutyl, cyclopentyl, or cyclohexyl, wherein each C₁₋₆ alkyl is optionally substituted with OH, NH₂, or —NHC(O)O-t-butyl, and wherein each C₃₋₈ cycloalkyl is optionally substituted with methyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is —C(O)OR^{4G5}, wherein R^{4G5} is methyl, ethyl, n-propyl, iso-propyl, n-butyl, iso-butyl, sec-butyl, or t-butyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is

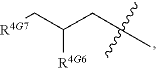

wherein
R^{4G6} and R^{4G7} are each independently H or —OR^{4G11}, wherein
at least one of R^{4G6} and R^{4G7} is —OR^{4G11}, and
each R^{4G11} is independently hexadecane, octadecane or benzyl.

In some embodiments, the compound can be represented by Formula (Ia), (Ib), (Ic), (Id), (Ie), (If), (Ig), (Ih) or (Ii), or a pharmaceutically acceptable salt thereof, wherein R^{4G} is methyl, ethyl, n-propyl, iso-propyl, n-butyl, pentyl, neopentyl, hexyl, 2,2-dimethylbutyl, 3,3-dimethylbutyl, 2-ethyl-butyl, octyl, dodecyl, hexadecyl, octadecyl, cyclobutyl, cyclopentyl, cyclohexyl, cycloheptyl, cyclooctyl,

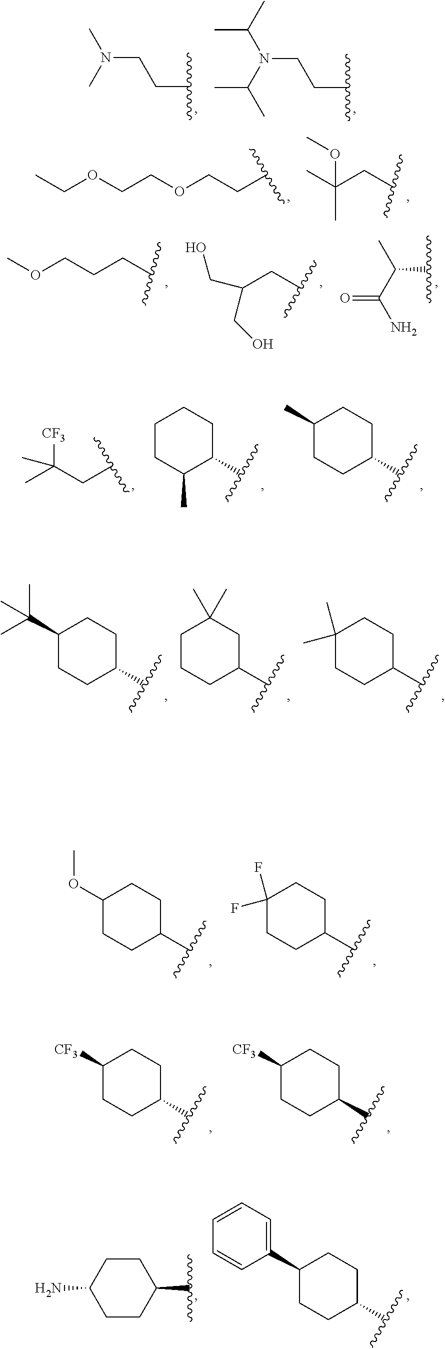

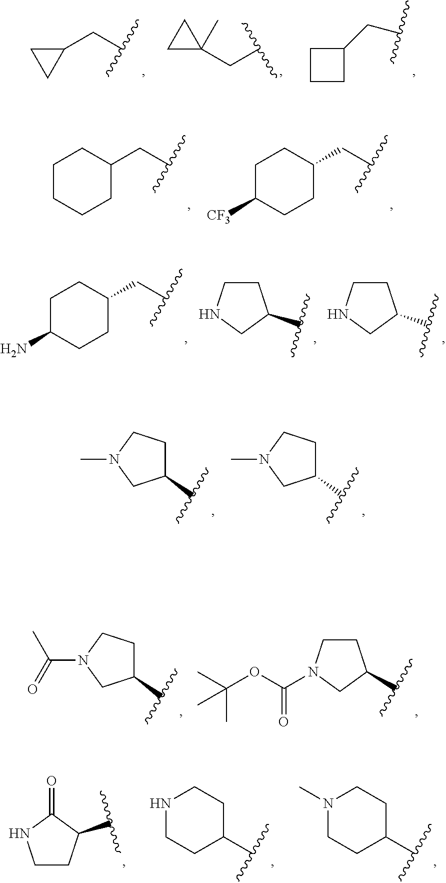

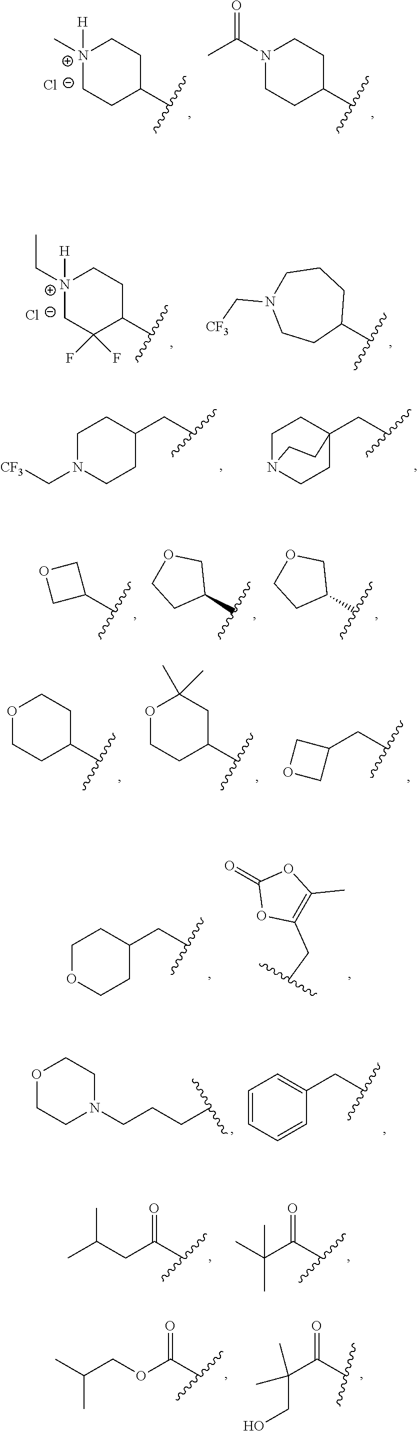

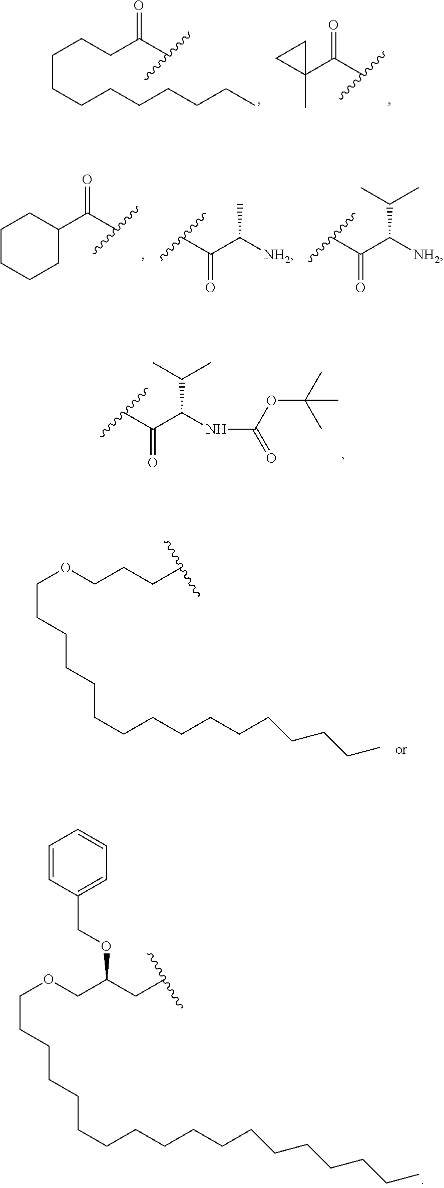

In some embodiments, the present disclosure provides a compound, or a pharmaceutically acceptable salt thereof, of Table 1A, Table 1B, Table 1C, Table 1D, Table 1E, Table 1F, Table 1G, Table 1H, Table 1I or Table 1J. In some embodiments, the compound is a compound, or a pharmaceutically salt thereof, of Table 1A. In some embodiments, the compound is a compound, or a pharmaceutically salt thereof, of Table 1B. In some embodiments, the compound is a compound, or a pharmaceutically salt thereof, of Table 1C. In some embodiments, the compound is a compound, or a pharmaceutically salt thereof, of Table 1D. In some embodiments, the compound is a compound, or a pharmaceutically salt thereof, of Table 1E. In some embodiments, the compound is a compound, or a pharmaceutically salt thereof, of Table 1F. In some embodiments, the compound is a compound, or a pharmaceutically salt thereof, of Table 1G. In some embodiments, the compound is a compound, or a pharmaceutically salt thereof, of Table 1H. In some embodiments, the compound is a compound, or a pharmaceutically salt thereof, of Table 1I. In some embodiments, the compound is a compound, or a pharmaceutically salt thereof, of Table 1J.

TABLE 1A

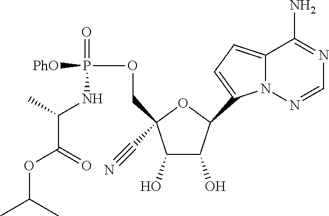 [SMILES: CC(C)OC(=O)[C@H](C)N[P@](=O)(OC[C@@]1(C#N)O[C@@H](c2ccc3c(N)ncnn23)[C@H](O)[C@@H]1O)Oc1ccccc1]

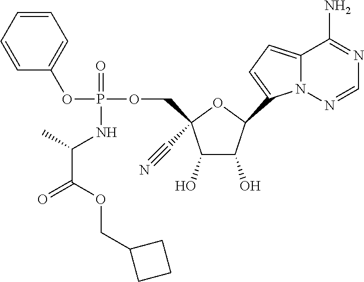 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCC1]

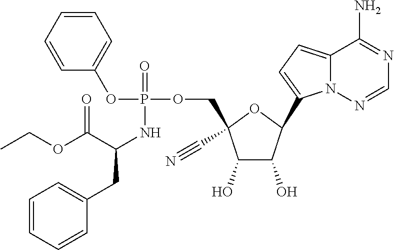 [SMILES: CCOC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

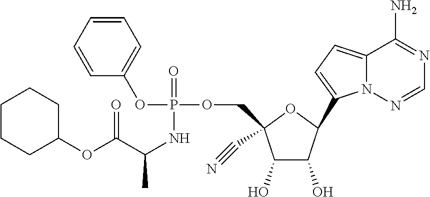 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

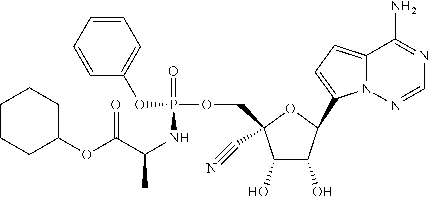 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCCCC1]

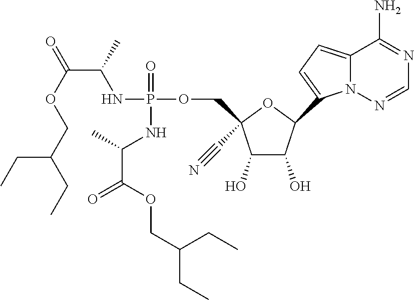 [SMILES: CCC(CC)COC(=O)[C@H](C)NP(=O)(N[C@@H](C)C(=O)OCC(CC)CC)OC[C@@]1(C#N)O[C@@H](c2ccc3c(N)ncnn23)[C@H](O)[C@@H]1O]

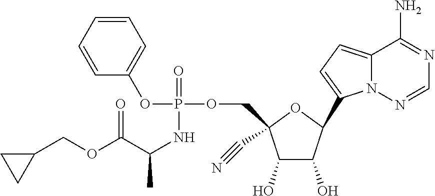 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CC1]

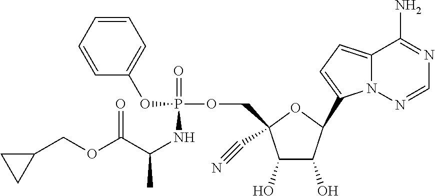 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CC1]

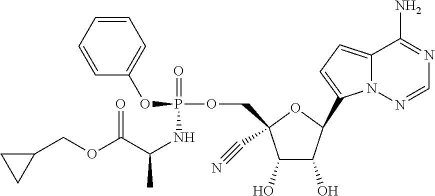 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC1CC1]

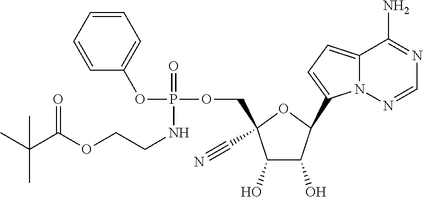 [SMILES: CC(C)(C)C(=O)OCCNP(=O)(OC[C@@]1(C#N)O[C@@H](c2ccc3c(N)ncnn23)[C@H](O)[C@@H]1O)Oc1ccccc1]

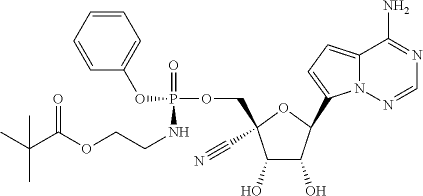 [SMILES: CC(C)(C)C(=O)OCCN[P@@](=O)(OC[C@@]1(C#N)O[C@@H](c2ccc3c(N)ncnn23)[C@H](O)[C@@H]1O)Oc1ccccc1]

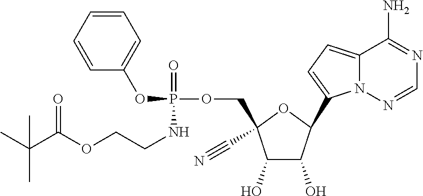 [SMILES: CC(C)(C)C(=O)OCCN[P@](=O)(OC[C@@]1(C#N)O[C@@H](c2ccc3c(N)ncnn23)[C@H](O)[C@@H]1O)Oc1ccccc1]

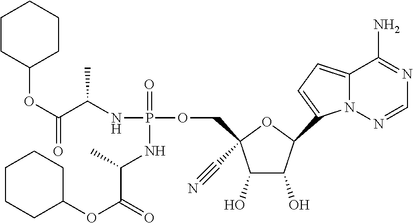 [SMILES: C[C@H](NP(=O)(N[C@@H](C)C(=O)OC1CCCCC1)OC[C@@]1(C#N)O[C@@H](c2ccc3c(N)ncnn23)[C@H](O)[C@@H]1O)C(=O)OC1CCCCC1]

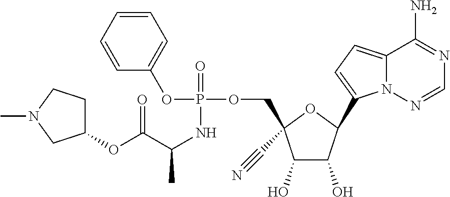 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCN(C)C1]

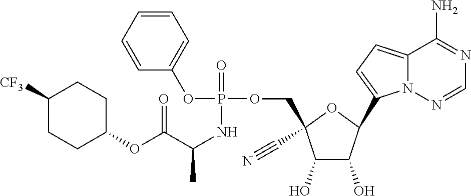 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

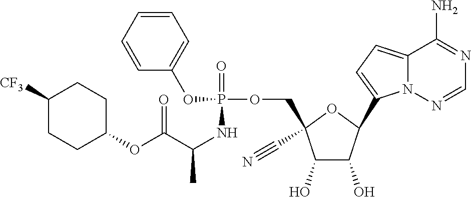 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

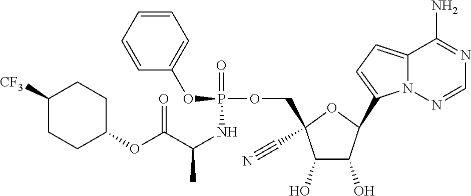 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

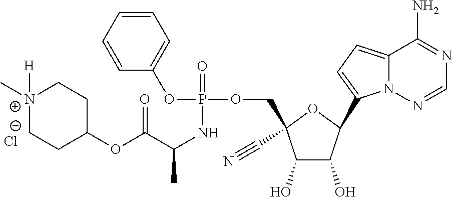 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CC[NH+](C)CC1 ; [Cl-]]

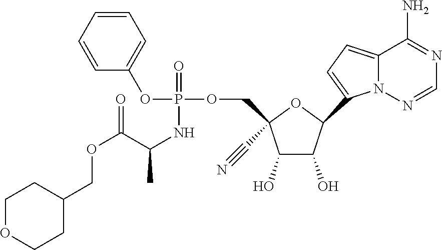 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCOCC1]

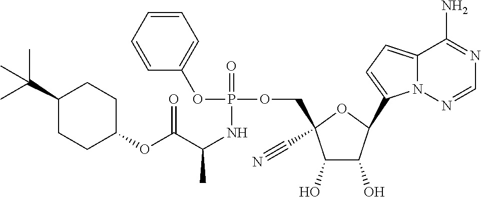 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

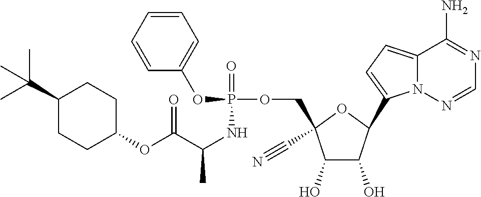 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

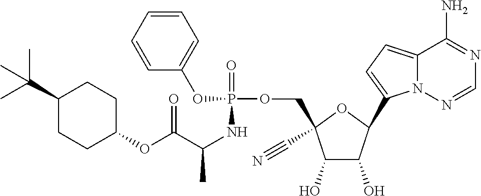 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

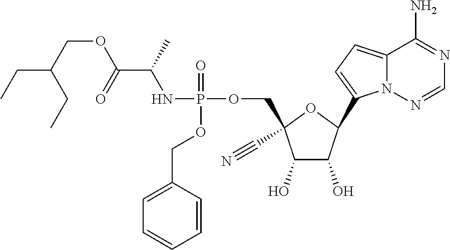 [SMILES: CCC(CC)COC(=O)[C@H](C)NP(=O)(OCc1ccccc1)OC[C@@]1(C#N)O[C@@H](c2ccc3c(N)ncnn23)[C@H](O)[C@@H]1O]

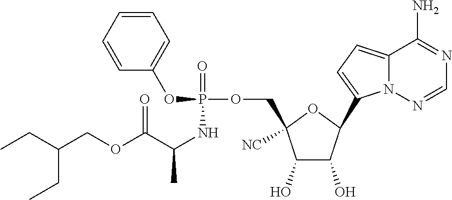 [SMILES: CCC(CC)COC(=O)[C@H](C)N[P@](=O)(OC[C@@]1(C#N)O[C@@H](c2ccc3c(N)ncnn23)[C@H](O)[C@@H]1O)Oc1ccccc1]

TABLE 1B

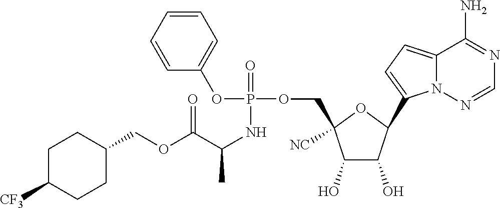 [SMILES: C[C@H](NP(=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1]

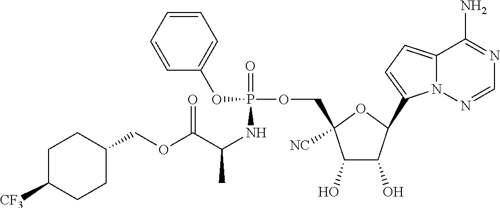 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1]

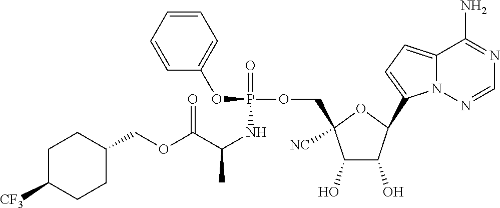 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1]

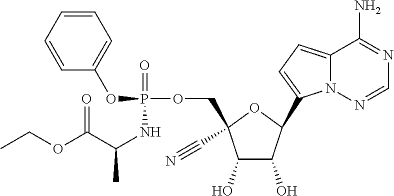 [SMILES: CCOC(=O)[C@H](C)N[P@](=O)(OC[C@@]1(C#N)O[C@@H](c2ccc3c(N)ncnn23)[C@H](O)[C@@H]1O)Oc1ccccc1]

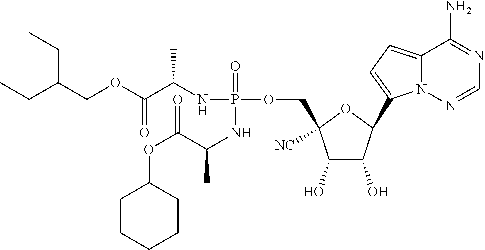 [SMILES: CCC(CC)COC(=O)[C@H](C)NP(=O)(N[C@@H](C)C(=O)OC1CCCCC1)OC[C@@]1(C#N)O[C@@H](c2ccc3c(N)ncnn23)[C@H](O)[C@@H]1O]

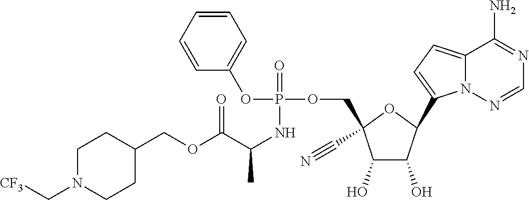 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCN(CC(F)(F)F)CC1]

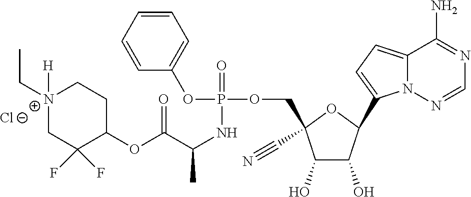 [SMILES: [Cl-] ; CC[NH+]1CCC(OC(=O)[C@H](C)NP(=O)(OC[C@@]2(C#N)O[C@@H](c3ccc4c(N)ncnn34)[C@H](O)[C@@H]2O)Oc2ccccc2)C(F)(F)C1]

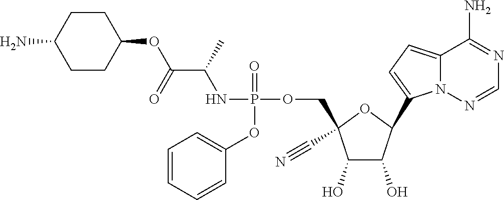 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](N)CC1]

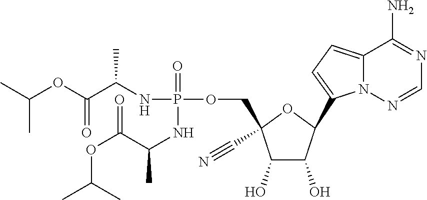 [SMILES: CC(C)OC(=O)[C@H](C)NP(=O)(N[C@@H](C)C(=O)OC(C)C)OC[C@@]1(C#N)O[C@@H](c2ccc3c(N)ncnn23)[C@H](O)[C@@H]1O]

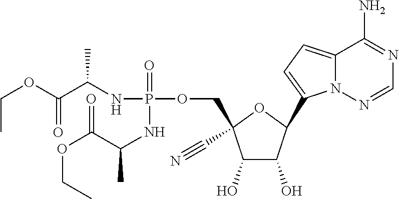 [SMILES: CCOC(=O)[C@H](C)NP(=O)(N[C@@H](C)C(=O)OCC)OC[C@@]1(C#N)O[C@@H](c2ccc3c(N)ncnn23)[C@H](O)[C@@H]1O]

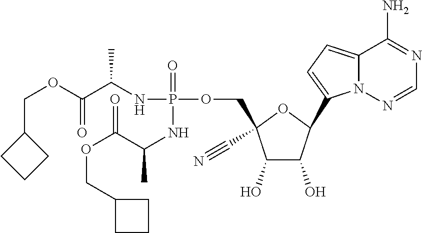 [SMILES: C[C@H](NP(=O)(N[C@@H](C)C(=O)OCC1CCC1)OC[C@@]1(C#N)O[C@@H](c2ccc3c(N)ncnn23)[C@H](O)[C@@H]1O)C(=O)OCC1CCC1]

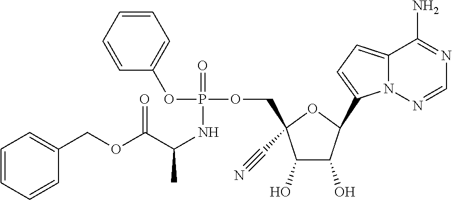 [SMILES: C[C@H](NP(=O)(OC[C@@]1(C#N)O[C@@H](c2ccc3c(N)ncnn23)[C@H](O)[C@@H]1O)Oc1ccccc1)C(=O)OCc1ccccc1]

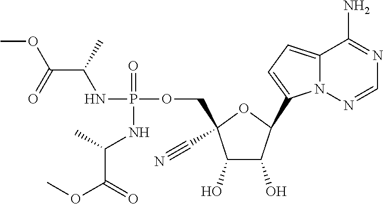 [SMILES: COC(=O)[C@H](C)NP(=O)(N[C@@H](C)C(=O)OC)OC[C@@]1(C#N)O[C@@H](c2ccc3c(N)ncnn23)[C@H](O)[C@@H]1O]

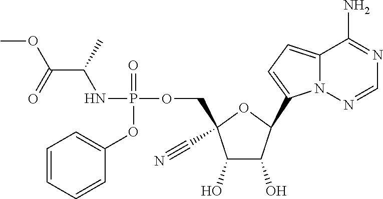 [SMILES: COC(=O)[C@H](C)NP(=O)(OC[C@@]1(C#N)O[C@@H](c2ccc3c(N)ncnn23)[C@H](O)[C@@H]1O)Oc1ccccc1]

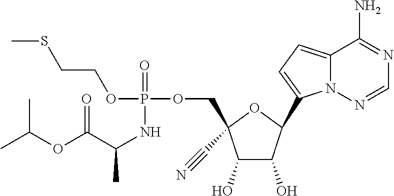 [SMILES: CSCCOP(=O)(N[C@@H](C)C(=O)OC(C)C)OC[C@@]1(C#N)O[C@@H](c2ccc3c(N)ncnn23)[C@H](O)[C@@H]1O]

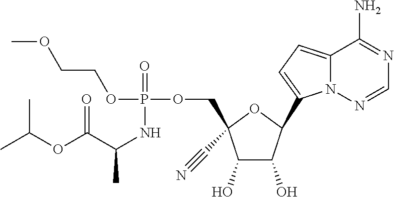 [SMILES: COCCOP(=O)(N[C@@H](C)C(=O)OC(C)C)OC[C@@]1(C#N)O[C@@H](c2ccc3c(N)ncnn23)[C@H](O)[C@@H]1O]

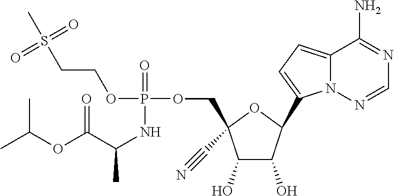 [SMILES: CC(C)OC(=O)[C@H](C)NP(=O)(OCCS(C)(=O)=O)OC[C@@]1(C#N)O[C@@H](c2ccc3c(N)ncnn23)[C@H](O)[C@@H]1O]

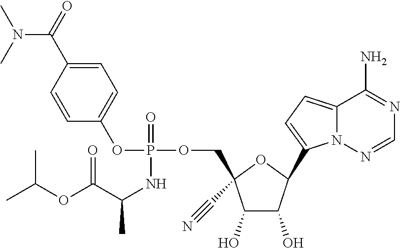 [SMILES: CC(C)OC(=O)[C@H](C)NP(=O)(OC[C@@]1(C#N)O[C@@H](c2ccc3c(N)ncnn23)[C@H](O)[C@@H]1O)Oc1ccc(C(=O)N(C)C)cc1]

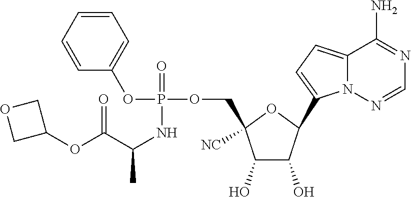 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1COC1]

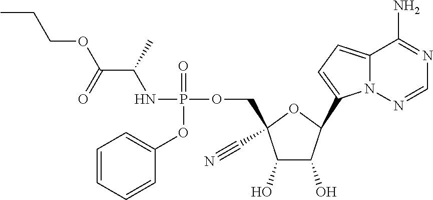 [SMILES: CCCOC(=O)[C@H](C)NP(=O)(OC[C@@]1(C#N)O[C@@H](c2ccc3c(N)ncnn23)[C@H](O)[C@@H]1O)Oc1ccccc1]

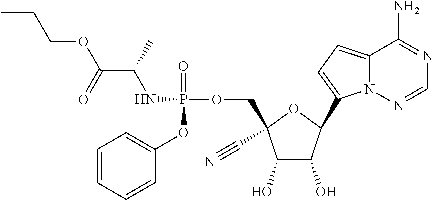 [SMILES: CCCOC(=O)[C@H](C)N[P@](=O)(OC[C@@]1(C#N)O[C@@H](c2ccc3c(N)ncnn23)[C@H](O)[C@@H]1O)Oc1ccccc1]

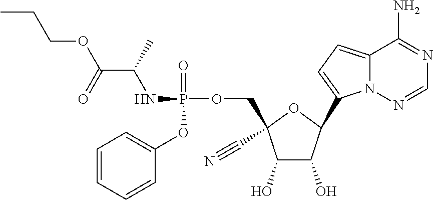 [SMILES: CCCOC(=O)[C@H](C)N[P@@](=O)(OC[C@@]1(C#N)O[C@@H](c2ccc3c(N)ncnn23)[C@H](O)[C@@H]1O)Oc1ccccc1]

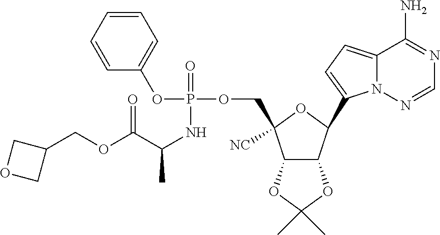 [SMILES: C[C@H](NP(=O)(OC[C@@]1(C#N)O[C@@H](c2ccc3c(N)ncnn23)[C@@H]2OC(C)(C)O[C@@H]21)Oc1ccccc1)C(=O)OCC1COC1]

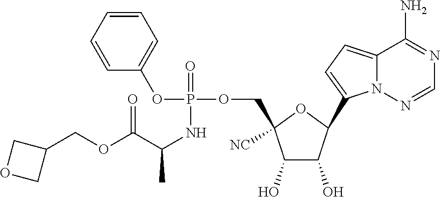 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1COC1]

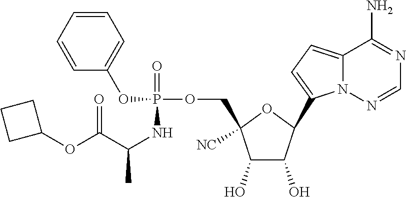 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCC1]

TABLE 1C

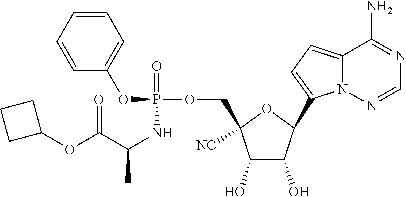 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCC1]

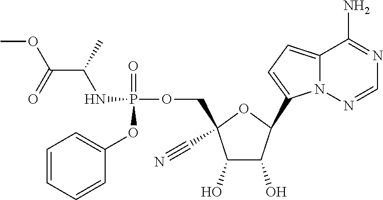 [SMILES: COC(=O)[C@H](C)N[P@](=O)(OC[C@@]1(C#N)O[C@@H](c2ccc3c(N)ncnn23)[C@H](O)[C@@H]1O)Oc1ccccc1]

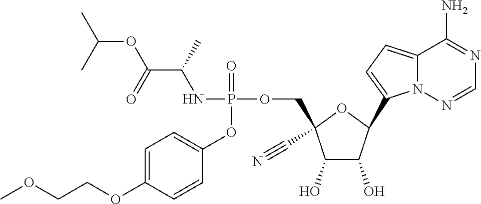 [SMILES: COCCOc1ccc(OP(=O)(N[C@@H](C)C(=O)OC(C)C)OC[C@@]2(C#N)O[C@@H](c3ccc4c(N)ncnn34)[C@H](O)[C@@H]2O)cc1]

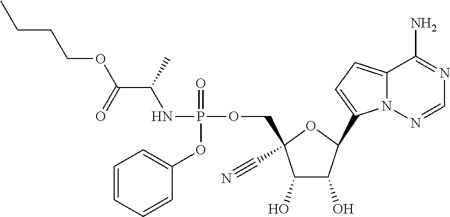 [SMILES: CCCCOC(=O)[C@H](C)NP(=O)(OC[C@@]1(C#N)O[C@@H](c2ccc3c(N)ncnn23)[C@H](O)[C@@H]1O)Oc1ccccc1]

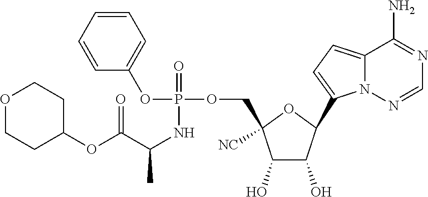 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCOCC1]

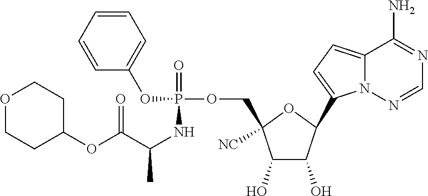 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCOCC1]

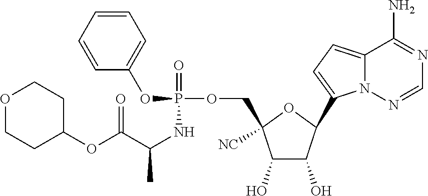 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCOCC1]

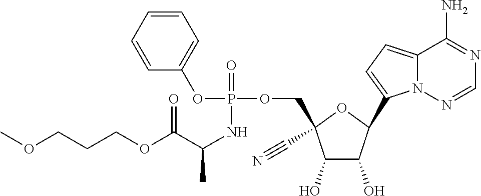 [SMILES: COCCCOC(=O)[C@H](C)NP(=O)(OC[C@@]1(C#N)O[C@@H](c2ccc3c(N)ncnn23)[C@H](O)[C@@H]1O)Oc1ccccc1]

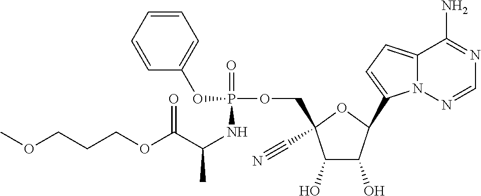 [SMILES: COCCCOC(=O)[C@H](C)N[P@@](=O)(OC[C@@]1(C#N)O[C@@H](c2ccc3c(N)ncnn23)[C@H](O)[C@@H]1O)Oc1ccccc1]

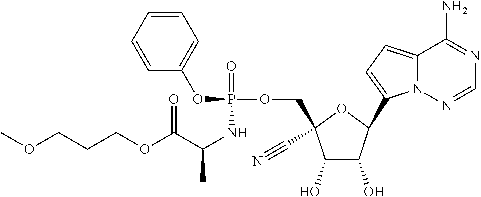 [SMILES: COCCCOC(=O)[C@H](C)N[P@](=O)(OC[C@@]1(C#N)O[C@@H](c2ccc3c(N)ncnn23)[C@H](O)[C@@H]1O)Oc1ccccc1]

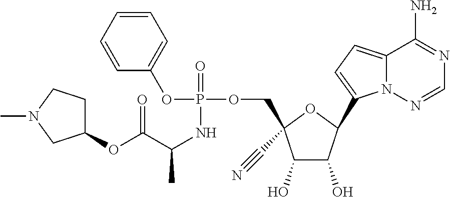 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCN(C)C1]

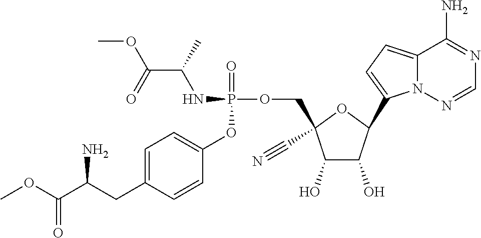 [SMILES: COC(=O)[C@H](C)N[P@@](=O)(OC[C@@]1(C#N)O[C@@H](c2ccc3c(N)ncnn23)[C@H](O)[C@@H]1O)Oc1ccc(C[C@H](N)C(=O)OC)cc1]

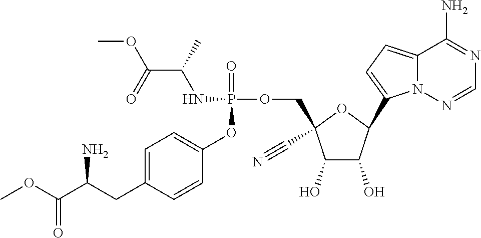 [SMILES: COC(=O)[C@H](C)N[P@](=O)(OC[C@@]1(C#N)O[C@@H](c2ccc3c(N)ncnn23)[C@H](O)[C@@H]1O)Oc1ccc(C[C@H](N)C(=O)OC)cc1]

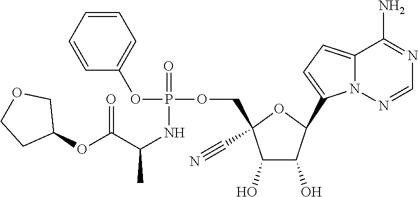 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCOC1]

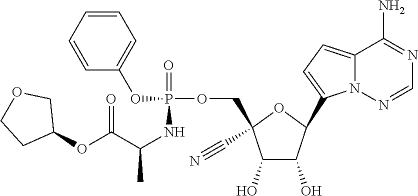 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CCOC1]

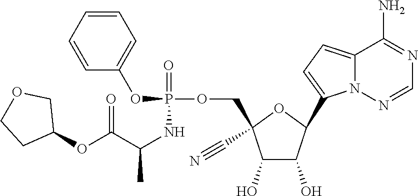 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CCOC1]

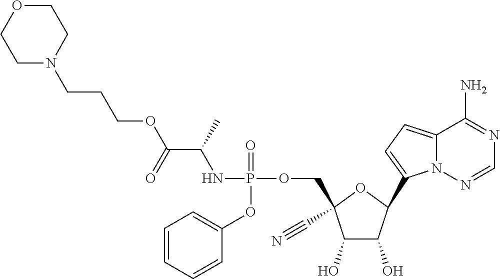 [SMILES: C[C@H](NP(=O)(OC[C@@]1(C#N)O[C@@H](c2ccc3c(N)ncnn23)[C@H](O)[C@@H]1O)Oc1ccccc1)C(=O)OCCCN1CCOCC1]

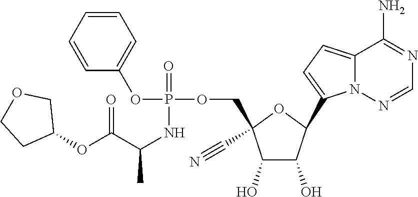 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCOC1]

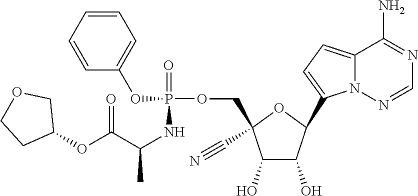 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@@H]1CCOC1]

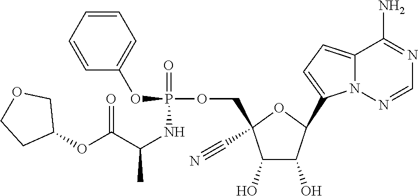 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@@H]1CCOC1]

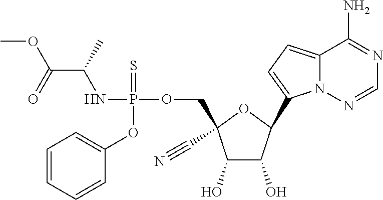 [SMILES: COC(=O)[C@H](C)NP(=S)(OC[C@@]1(C#N)O[C@@H](c2ccc3c(N)ncnn23)[C@H](O)[C@@H]1O)Oc1ccccc1]

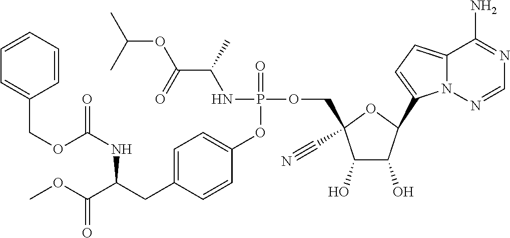 [SMILES: COC(=O)[C@H](Cc1ccc(OP(=O)(N[C@@H](C)C(=O)OC(C)C)OC[C@@]2(C#N)O[C@@H](c3ccc4c(N)ncnn34)[C@H](O)[C@@H]2O)cc1)NC(=O)OCc1ccccc1]

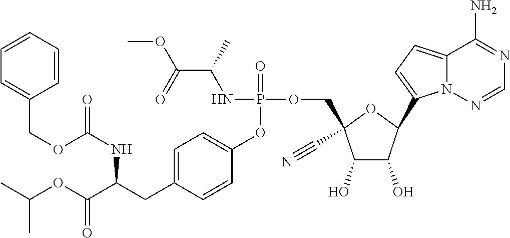 [SMILES: COC(=O)[C@H](C)NP(=O)(OC[C@@]1(C#N)O[C@@H](c2ccc3c(N)ncnn23)[C@H](O)[C@@H]1O)Oc1ccc(C[C@H](NC(=O)OCc2ccccc2)C(=O)OC(C)C)cc1]

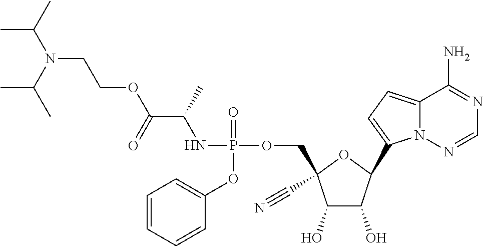 [SMILES: CC(C)N(CCOC(=O)[C@H](C)NP(=O)(OC[C@@]1(C#N)O[C@@H](c2ccc3c(N)ncnn23)[C@H](O)[C@@H]1O)Oc1ccccc1)C(C)C]

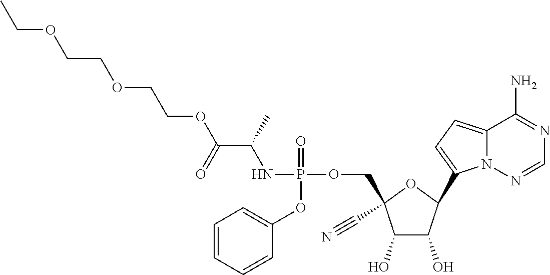 [SMILES: CCOCCOCCOC(=O)[C@H](C)NP(=O)(OC[C@@]1(C#N)O[C@@H](c2ccc3c(N)ncnn23)[C@H](O)[C@@H]1O)Oc1ccccc1]

TABLE 1D

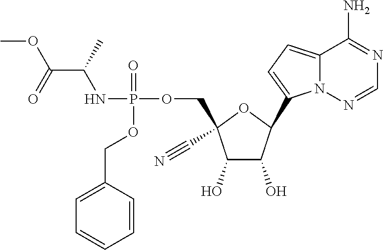 [SMILES: COC(=O)[C@H](C)NP(=O)(OCc1ccccc1)OC[C@@]1(C#N)O[C@@H](c2ccc3c(N)ncnn23)[C@H](O)[C@@H]1O]

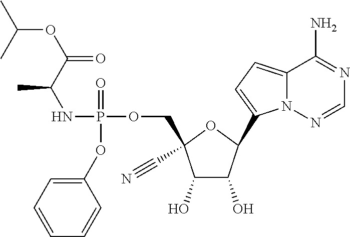 [SMILES: CC(C)OC(=O)[C@H](C)NP(=O)(OC[C@@]1(C#N)O[C@@H](c2ccc3c(N)ncnn23)[C@H](O)[C@@H]1O)Oc1ccccc1]

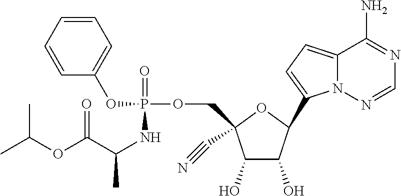 [SMILES: CC(C)OC(=O)[C@H](C)N[P@@](=O)(OC[C@@]1(C#N)O[C@@H](c2ccc3c(N)ncnn23)[C@H](O)[C@@H]1O)Oc1ccccc1]

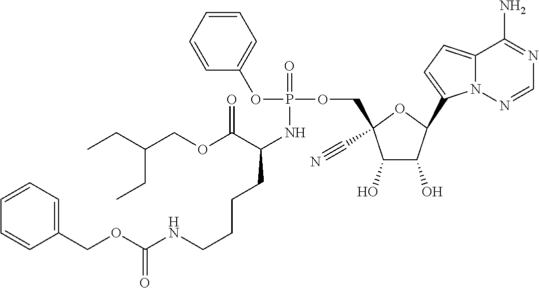 [SMILES: CCC(CC)COC(=O)[C@H](CCCCNC(=O)OCc1ccccc1)NP(=O)(OC[C@@]1(C#N)O[C@@H](c2ccc3c(N)ncnn23)[C@H](O)[C@@H]1O)Oc1ccccc1]

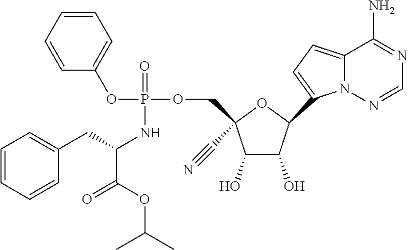 [SMILES: CC(C)OC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

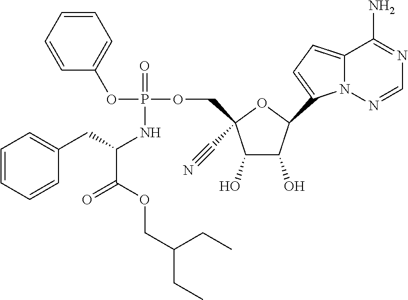 [SMILES: CCC(CC)COC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

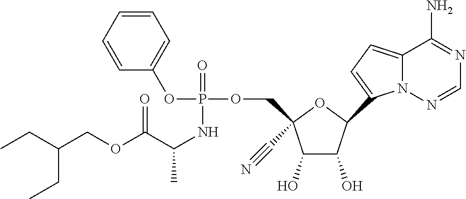 [SMILES: CCC(CC)COC(=O)[C@@H](C)NP(=O)(OC[C@@]1(C#N)O[C@@H](c2ccc3c(N)ncnn23)[C@H](O)[C@@H]1O)Oc1ccccc1]

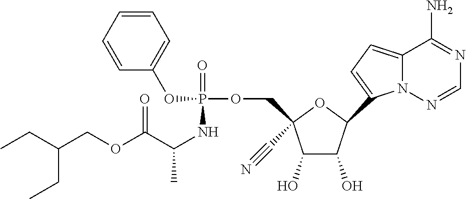 [SMILES: CCC(CC)COC(=O)[C@@H](C)N[P@@](=O)(OC[C@@]1(C#N)O[C@@H](c2ccc3c(N)ncnn23)[C@H](O)[C@@H]1O)Oc1ccccc1]

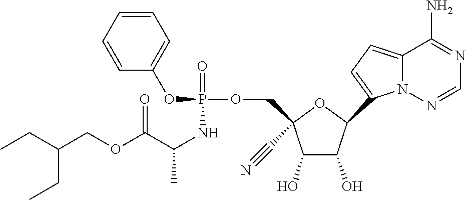 [SMILES: CCC(CC)COC(=O)[C@@H](C)N[P@](=O)(OC[C@@]1(C#N)O[C@@H](c2ccc3c(N)ncnn23)[C@H](O)[C@@H]1O)Oc1ccccc1]

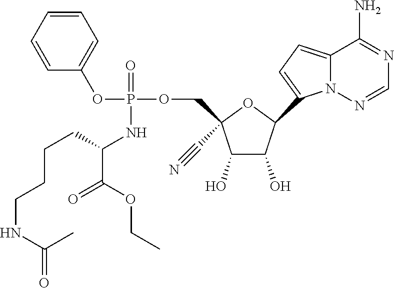 [SMILES: CCOC(=O)[C@H](CCCCNC(C)=O)NP(=O)(OC[C@@]1(C#N)O[C@@H](c2ccc3c(N)ncnn23)[C@H](O)[C@@H]1O)Oc1ccccc1]

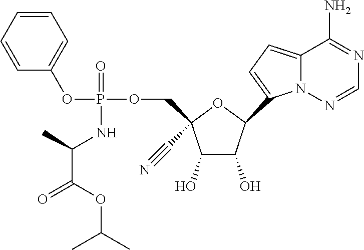 [SMILES: CC(C)OC(=O)[C@@H](C)NP(=O)(OC[C@@]1(C#N)O[C@@H](c2ccc3c(N)ncnn23)[C@H](O)[C@@H]1O)Oc1ccccc1]

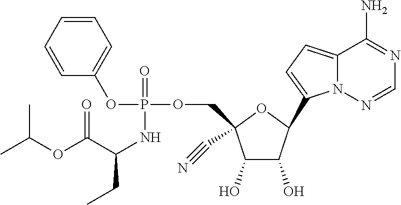 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC(C)C]

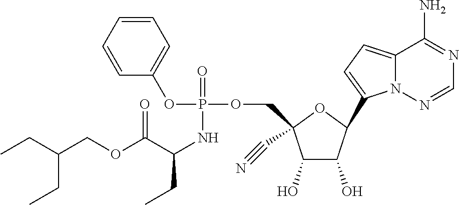 [SMILES: CCC(CC)COC(=O)[C@H](CC)NP(=O)(OC[C@@]1(C#N)O[C@@H](c2ccc3c(N)ncnn23)[C@H](O)[C@@H]1O)Oc1ccccc1]

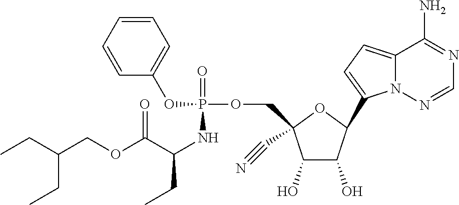 [SMILES: CCC(CC)COC(=O)[C@H](CC)N[P@@](=O)(OC[C@@]1(C#N)O[C@@H](c2ccc3c(N)ncnn23)[C@H](O)[C@@H]1O)Oc1ccccc1]

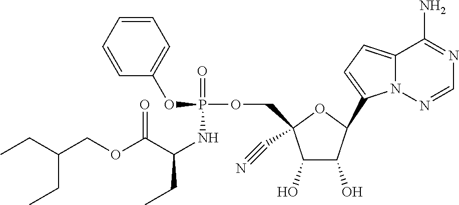 [SMILES: CCC(CC)COC(=O)[C@H](CC)N[P@](=O)(OC[C@@]1(C#N)O[C@@H](c2ccc3c(N)ncnn23)[C@H](O)[C@@H]1O)Oc1ccccc1]

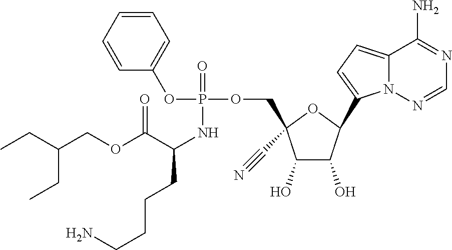 [SMILES: CCC(CC)COC(=O)[C@H](CCCCN)NP(=O)(OC[C@@]1(C#N)O[C@@H](c2ccc3c(N)ncnn23)[C@H](O)[C@@H]1O)Oc1ccccc1]

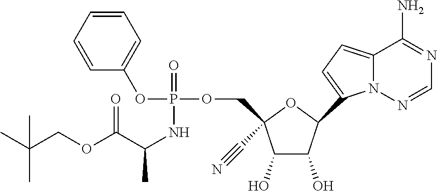 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC(C)(C)C]

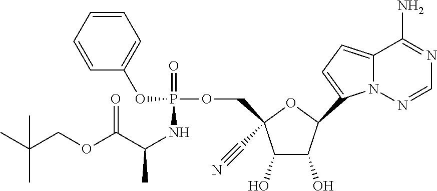 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC(C)(C)C]

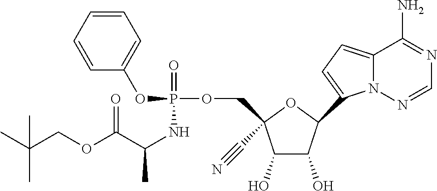 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC(C)(C)C]

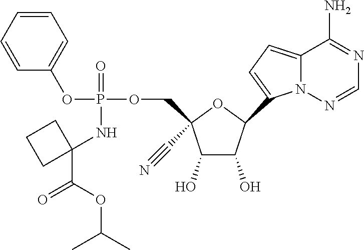 [SMILES: CC(C)OC(=O)C1(NP(=O)(OC[C@@]2(C#N)O[C@@H](c3ccc4c(N)ncnn34)[C@H](O)[C@@H]2O)Oc2ccccc2)CCC1]

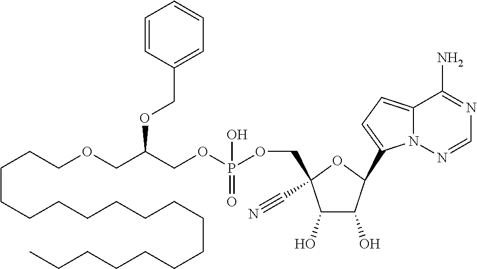 [SMILES: CCCCCCCCCCCCCCCCCCOC[C@H](COP(=O)(O)OC[C@@]1(C#N)O[C@@H](c2ccc3c(N)ncnn23)[C@H](O)[C@@H]1O)OCc1ccccc1]

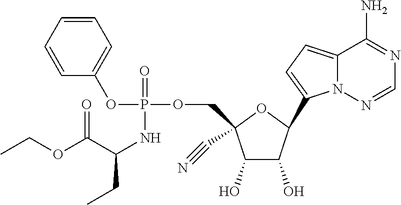 [SMILES: CCOC(=O)[C@H](CC)NP(=O)(OC[C@@]1(C#N)O[C@@H](c2ccc3c(N)ncnn23)[C@H](O)[C@@H]1O)Oc1ccccc1]

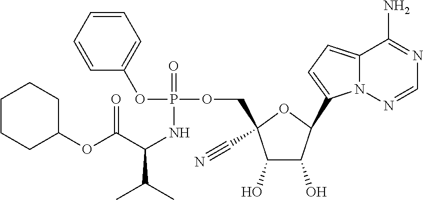 [SMILES: CC(C)[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

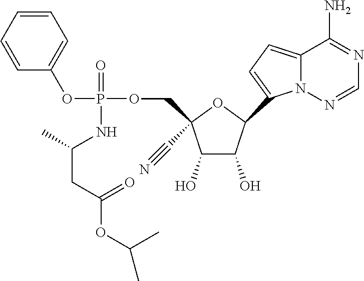 [SMILES: CC(C)OC(=O)C[C@H](C)NP(=O)(OC[C@@]1(C#N)O[C@@H](c2ccc3c(N)ncnn23)[C@H](O)[C@@H]1O)Oc1ccccc1]

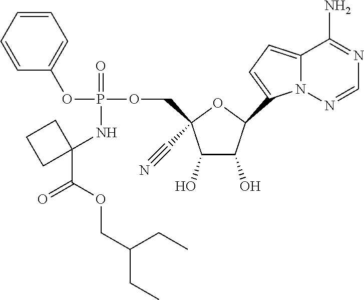 [SMILES: CCC(CC)COC(=O)C1(NP(=O)(OC[C@@]2(C#N)O[C@@H](c3ccc4c(N)ncnn34)[C@H](O)[C@@H]2O)Oc2ccccc2)CCC1]

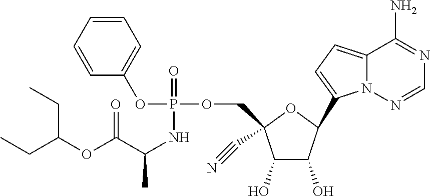 [SMILES: CCC(CC)OC(=O)[C@H](C)NP(=O)(OC[C@@]1(C#N)O[C@@H](c2ccc3c(N)ncnn23)[C@H](O)[C@@H]1O)Oc1ccccc1]

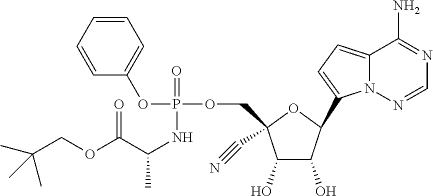 [SMILES: C[C@@H](NP(=O)(OC[C@@]1(C#N)O[C@@H](c2ccc3c(N)ncnn23)[C@H](O)[C@@H]1O)Oc1ccccc1)C(=O)OCC(C)(C)C]

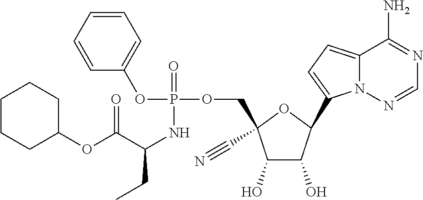 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

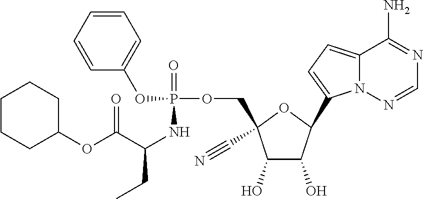 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OC1CCCCC1]

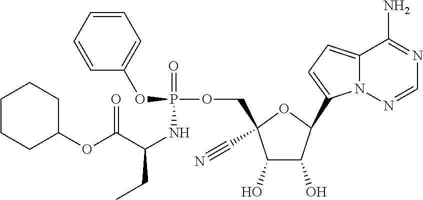 [SMILES: CC[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]

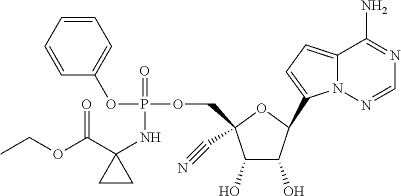 [SMILES: CCOC(=O)C1(NP(=O)(OC[C@@]2(C#N)O[C@@H](c3ccc4c(N)ncnn34)[C@H](O)[C@@H]2O)Oc2ccccc2)CC1]

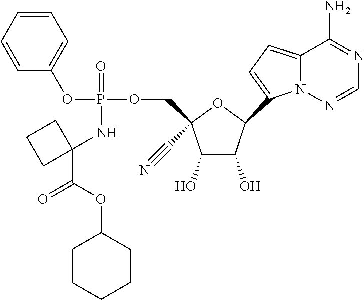 [SMILES: N#C[C@]1(COP(=O)(NC2(C(=O)OC3CCCCC3)CCC2)Oc2ccccc2)O[C@@H](c2ccc3c(N)ncnn23)[C@H](O)[C@@H]1O]

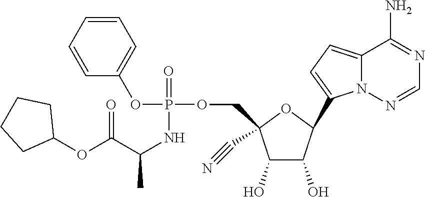 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCC1]

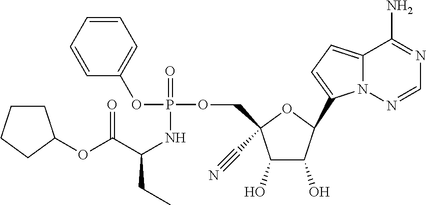 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC1CCCC1]

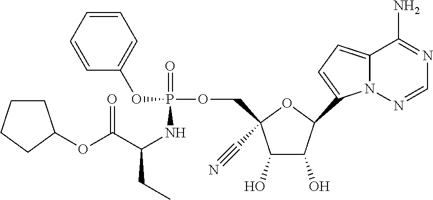 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OC1CCCC1]

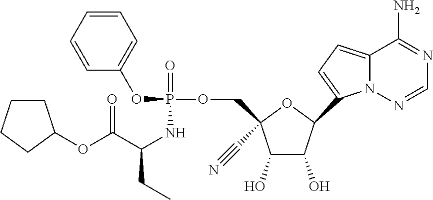 [SMILES: CC[C@H](N[P@](=O)(OC[C@@]1(C#N)O[C@@H](c2ccc3c(N)ncnn23)[C@H](O)[C@@H]1O)Oc1ccccc1)C(=O)OC1CCCC1]

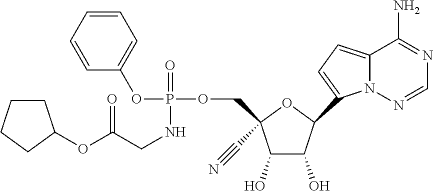 [SMILES: N#C[C@]1(COP(=O)(NCC(=O)OC2CCCC2)Oc2ccccc2)O[C@@H](c2ccc3c(N)ncnn23)[C@H](O)[C@@H]1O]

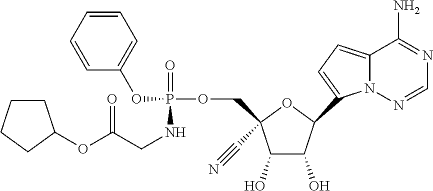 [SMILES: N#C[C@]1(CO[P@](=O)(NCC(=O)OC2CCCC2)Oc2ccccc2)O[C@@H](c2ccc3c(N)ncnn23)[C@H](O)[C@@H]1O]

TABLE 1E

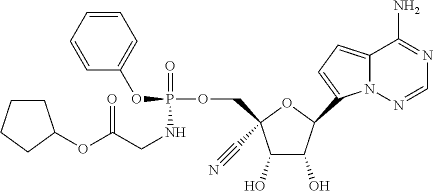 [SMILES: N#C[C@]1(CO[P@@](=O)(NCC(=O)OC2CCCC2)Oc2ccccc2)O[C@@H](c2ccc3c(N)ncnn23)[C@H](O)[C@@H]1O]

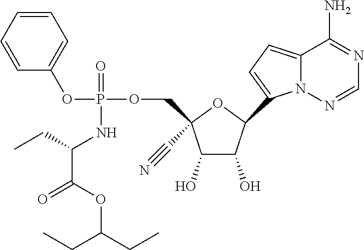 [SMILES: CCC(CC)OC(=O)[C@H](CC)NP(=O)(OC[C@@]1(C#N)O[C@@H](c2ccc3c(N)ncnn23)[C@H](O)[C@@H]1O)Oc1ccccc1]

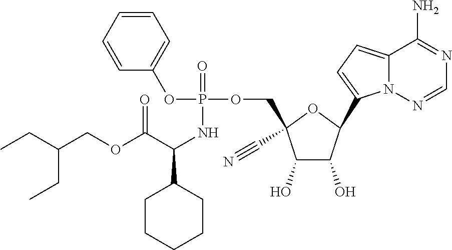 [SMILES: CCC(CC)COC(=O)[C@@H](NP(=O)(OC[C@@]1(C#N)O[C@@H](c2ccc3c(N)ncnn23)[C@H](O)[C@@H]1O)Oc1ccccc1)C1CCCCC1]

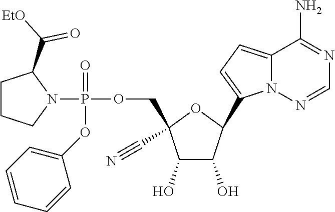 [SMILES: CCOC(=O)[C@@H]1CCCN1P(=O)(OC[C@@]1(C#N)O[C@@H](c2ccc3c(N)ncnn23)[C@H](O)[C@@H]1O)Oc1ccccc1]

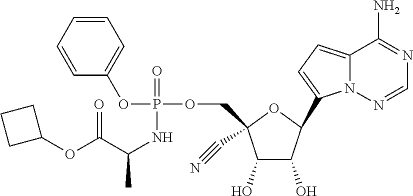 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCC1]

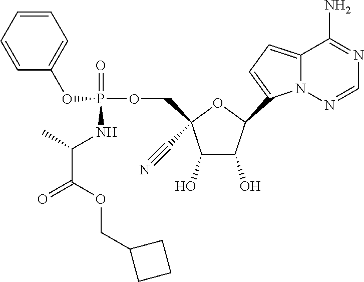 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CCC1]

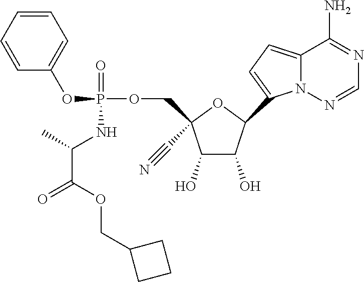 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC1CCC1]

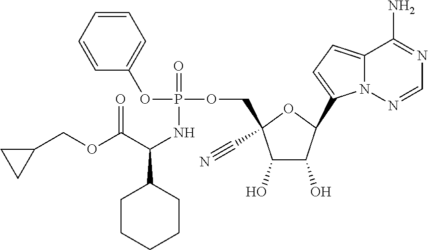 [SMILES: N#C[C@]1(COP(=O)(N[C@H](C(=O)OCC2CC2)C2CCCCC2)Oc2ccccc2)O[C@@H](c2ccc3c(N)ncnn23)[C@H](O)[C@@H]1O]

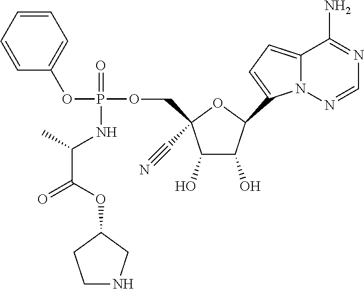 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCNC1]

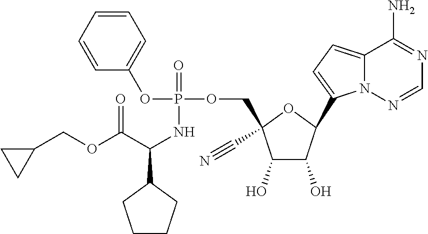 [SMILES: N#C[C@]1(COP(=O)(N[C@H](C(=O)OCC2CC2)C2CCCC2)Oc2ccccc2)O[C@@H](c2ccc3c(N)ncnn23)[C@H](O)[C@@H]1O]

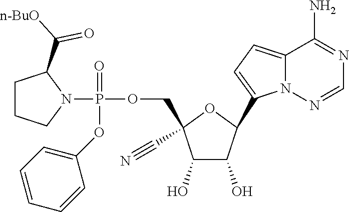

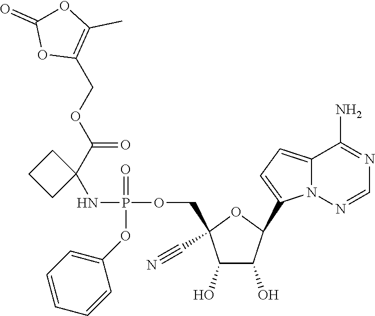 [SMILES: Cc1oc(=O)oc1COC(=O)C1(NP(=O)(OC[C@@]2(C#N)O[C@@H](c3ccc4c(N)ncnn34)[C@H](O)[C@@H]2O)Oc2ccccc2)CCC1]

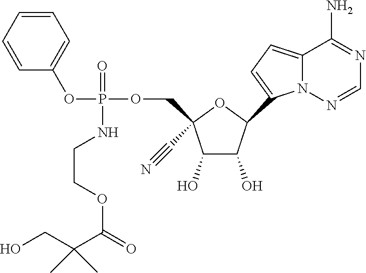 [SMILES: CC(C)(CO)C(=O)OCCNP(=O)(OC[C@@]1(C#N)O[C@@H](c2ccc3c(N)ncnn23)[C@H](O)[C@@H]1O)Oc1ccccc1]

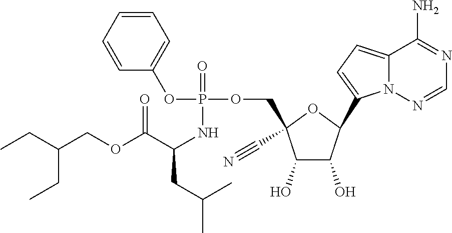 [SMILES: CCC(CC)COC(=O)[C@H](CC(C)C)NP(=O)(OC[C@@]1(C#N)O[C@@H](c2ccc3c(N)ncnn23)[C@H](O)[C@@H]1O)Oc1ccccc1]

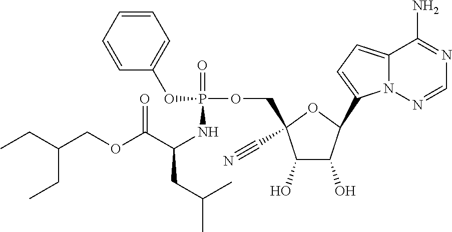 [SMILES: CCC(CC)COC(=O)[C@H](CC(C)C)N[P@@](=O)(OC[C@@]1(C#N)O[C@@H](c2ccc3c(N)ncnn23)[C@H](O)[C@@H]1O)Oc1ccccc1]

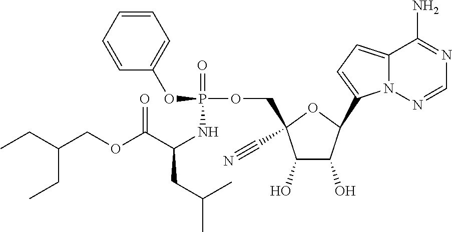 [SMILES: CCC(CC)COC(=O)[C@H](CC(C)C)N[P@](=O)(OC[C@@]1(C#N)O[C@@H](c2ccc3c(N)ncnn23)[C@H](O)[C@@H]1O)Oc1ccccc1]

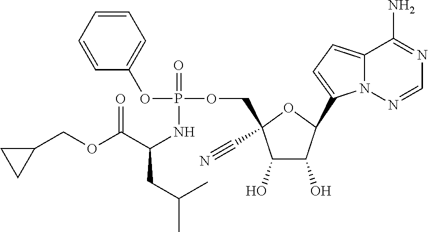 [SMILES: CC(C)C[C@H](NP(=O)(OC[C@@]1(C#N)O[C@@H](c2ccc3c(N)ncnn23)[C@H](O)[C@@H]1O)Oc1ccccc1)C(=O)OCC1CC1]

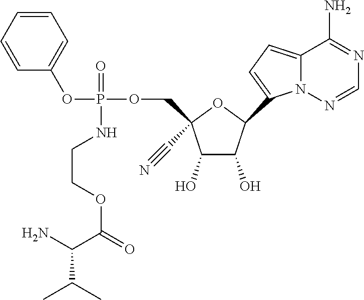 [SMILES: CC(C)[C@H](N)C(=O)OCCNP(=O)(OC[C@@]1(C#N)O[C@@H](c2ccc3c(N)ncnn23)[C@H](O)[C@@H]1O)Oc1ccccc1]

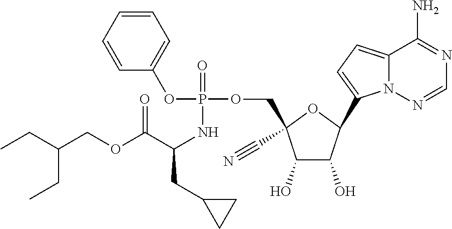 [SMILES: CCC(CC)COC(=O)[C@H](CC1CC1)NP(=O)(OC[C@@]1(C#N)O[C@@H](c2ccc3c(N)ncnn23)[C@H](O)[C@@H]1O)Oc1ccccc1]

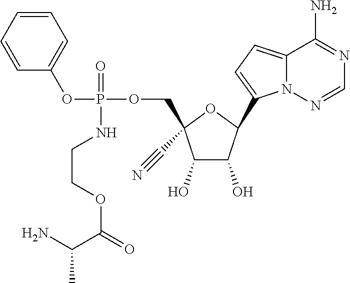 [SMILES: C[C@H](N)C(=O)OCCNP(=O)(OC[C@@]1(C#N)O[C@@H](c2ccc3c(N)ncnn23)[C@H](O)[C@@H]1O)Oc1ccccc1]

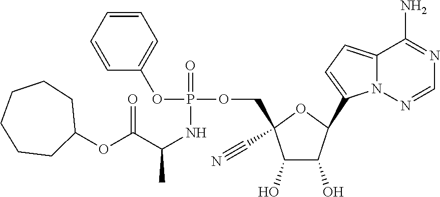 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCCC1]

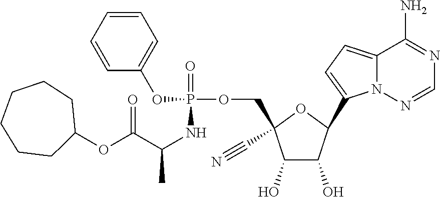 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCCCCC1]

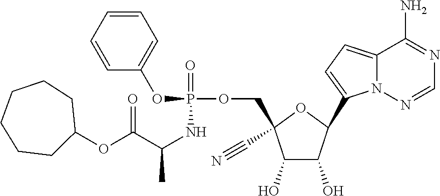 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCCC1]

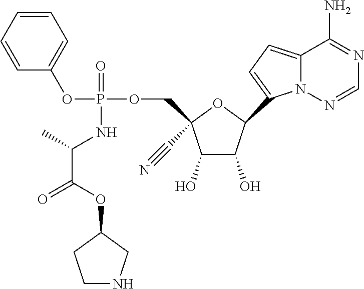 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCNC1]

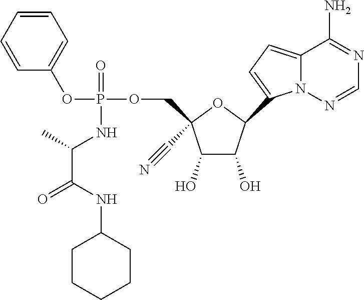 [SMILES: C[C@H](NP(=O)(OC[C@@]1(C#N)O[C@@H](c2ccc3c(N)ncnn23)[C@H](O)[C@@H]1O)Oc1ccccc1)C(=O)NC1CCCCC1]

TABLE 1F

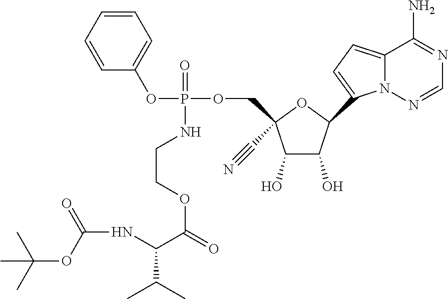 [SMILES: CC(C)[C@H](NC(=O)OC(C)(C)C)C(=O)OCCNP(=O)(OC[C@@]1(C#N)O[C@@H](c2ccc3c(N)ncnn23)[C@H](O)[C@@H]1O)Oc1ccccc1]

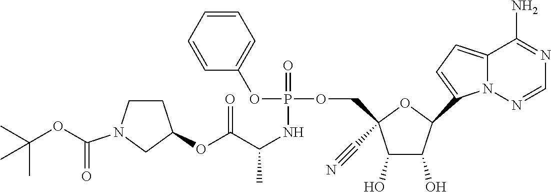 [SMILES: C[C@@H](NP(=O)(OC[C@@]1(C#N)O[C@@H](c2ccc3c(N)ncnn23)[C@H](O)[C@@H]1O)Oc1ccccc1)C(=O)O[C@@H]1CCN(C(=O)OC(C)(C)C)C1]

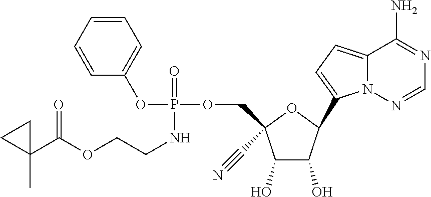 [SMILES: CC1(C(=O)OCCNP(=O)(OC[C@@]2(C#N)O[C@@H](c3ccc4c(N)ncnn34)[C@H](O)[C@@H]2O)Oc2ccccc2)CC1]

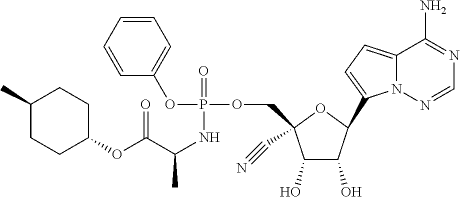 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1]

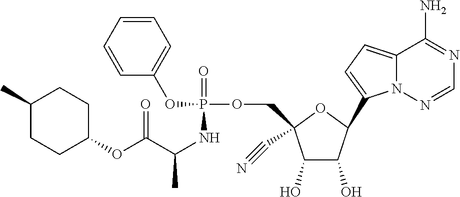 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1]

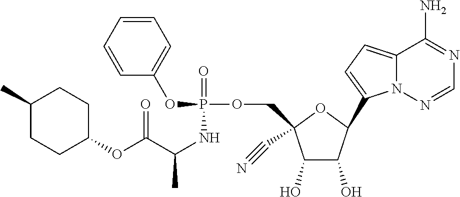 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1]

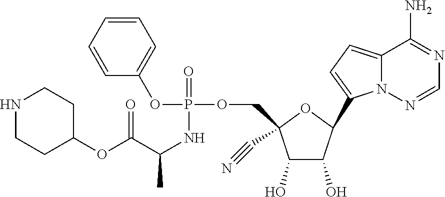 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCNCC1]

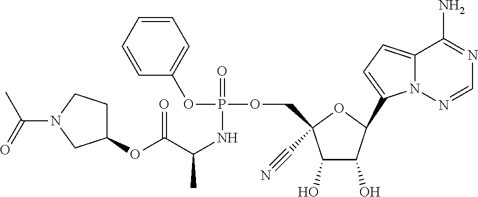 [SMILES: CC(=O)N1CC[C@@H](OC(=O)[C@H](C)NP(=O)(OC[C@@]2(C#N)O[C@@H](c3ccc4c(N)ncnn34)[C@H](O)[C@@H]2O)Oc2ccccc2)C1]

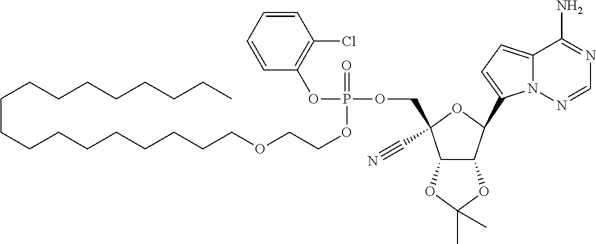 [SMILES: CCCCCCCCCCCCCCCCCCOCCOP(=O)(OC[C@@]1(C#N)O[C@@H](c2ccc3c(N)ncnn23)[C@@H]2OC(C)(C)O[C@@H]21)Oc1ccccc1Cl]

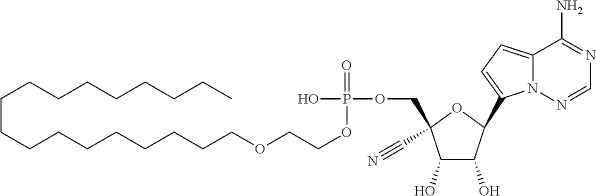 [SMILES: CCCCCCCCCCCCCCCCCCOCCOP(=O)(O)OC[C@@]1(C#N)O[C@@H](c2ccc3c(N)ncnn23)[C@H](O)[C@@H]1O]

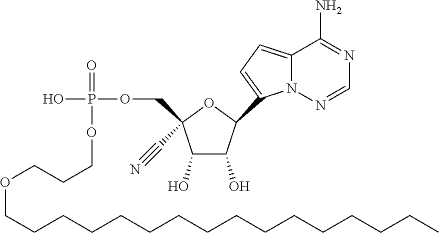 [SMILES: CCCCCCCCCCCCCCCCOCCCOP(=O)(O)OC[C@@]1(C#N)O[C@@H](c2ccc3c(N)ncnn23)[C@H](O)[C@@H]1O]

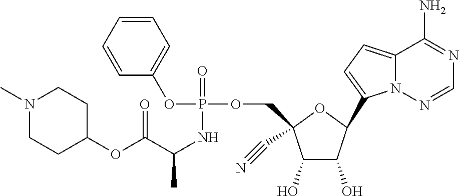 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCN(C)CC1]

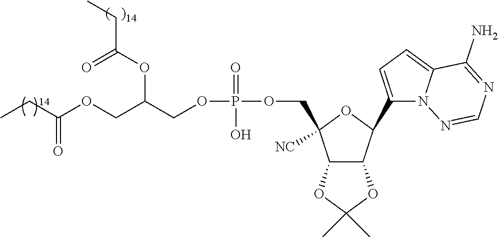

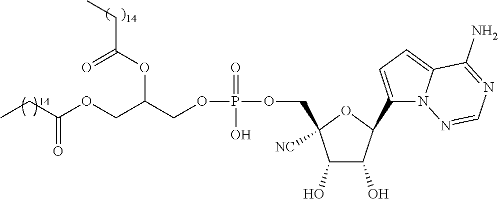

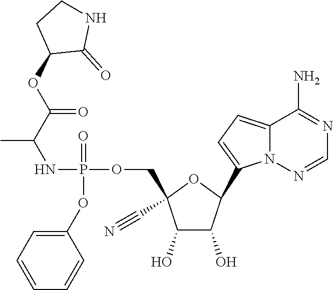 [SMILES: CC(NP(=O)(OC[C@@]1(C#N)O[C@@H](c2ccc3c(N)ncnn23)[C@H](O)[C@@H]1O)Oc1ccccc1)C(=O)O[C@H]1CCNC1=O]

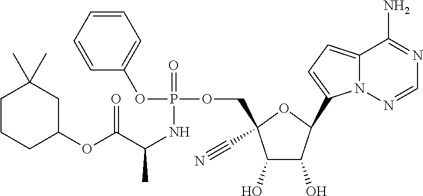 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCC(C)(C)C1]

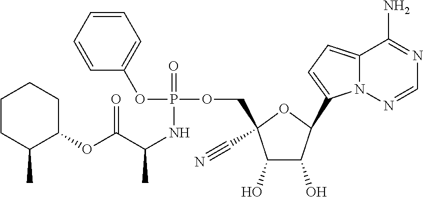 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCCC[C@@H]1C]

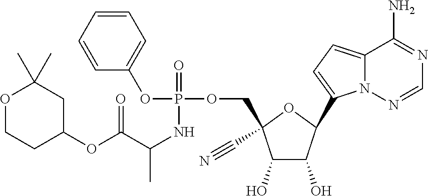 [SMILES: CC(NP(=O)(OC[C@@]1(C#N)O[C@@H](c2ccc3c(N)ncnn23)[C@H](O)[C@@H]1O)Oc1ccccc1)C(=O)OC1CCOC(C)(C)C1]

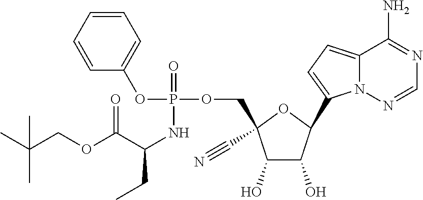 [SMILES: CC[C@H](NP(=O)(OC[C@@]1(C#N)O[C@@H](c2ccc3c(N)ncnn23)[C@H](O)[C@@H]1O)Oc1ccccc1)C(=O)OCC(C)(C)C]

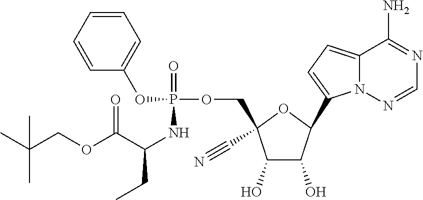 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OCC(C)(C)C]

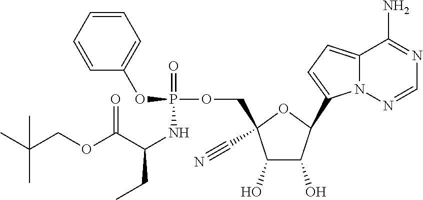 [SMILES: CC[C@H](N[P@](=O)(OC[C@@]1(C#N)O[C@@H](c2ccc3c(N)ncnn23)[C@H](O)[C@@H]1O)Oc1ccccc1)C(=O)OCC(C)(C)C]

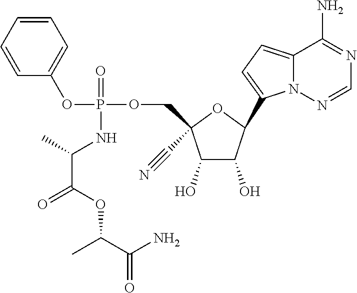 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H](C)C(N)=O]

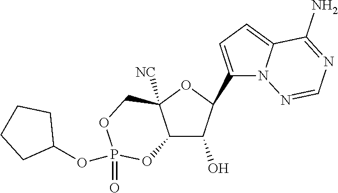

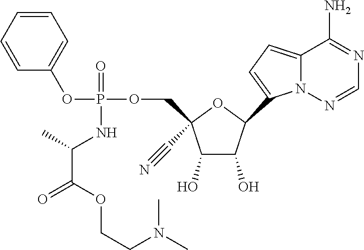 [SMILES: C[C@H](NP(=O)(OC[C@@]1(C#N)O[C@@H](c2ccc3c(N)ncnn23)[C@H](O)[C@@H]1O)Oc1ccccc1)C(=O)OCCN(C)C]

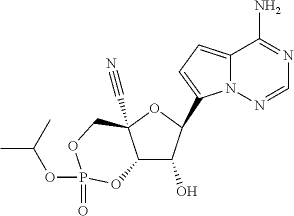

TABLE 1G

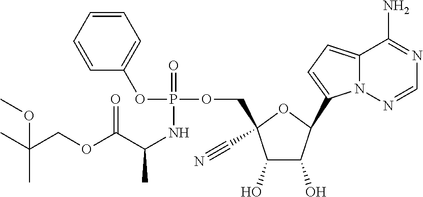 [SMILES: COC(C)(C)COC(=O)[C@H](C)NP(=O)(OC[C@@]1(C#N)O[C@@H](c2ccc3c(N)ncnn23)[C@H](O)[C@@H]1O)Oc1ccccc1]

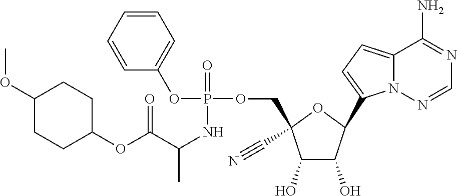 [SMILES: COC1CCC(OC(=O)C(C)NP(=O)(OC[C@@]2(C#N)O[C@@H](c3ccc4c(N)ncnn34)[C@H](O)[C@@H]2O)Oc2ccccc2)CC1]

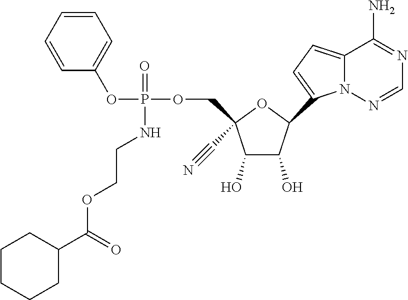 [SMILES: N#C[C@]1(COP(=O)(NCCOC(=O)C2CCCCC2)Oc2ccccc2)O[C@@H](c2ccc3c(N)ncnn23)[C@H](O)[C@@H]1O]

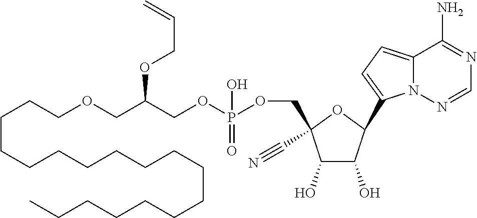 [SMILES: C=CCO[C@H](COCCCCCCCCCCCCCCCCCC)COP(=O)(O)OC[C@@]1(C#N)O[C@@H](c2ccc3c(N)ncnn23)[C@H](O)[C@@H]1O]

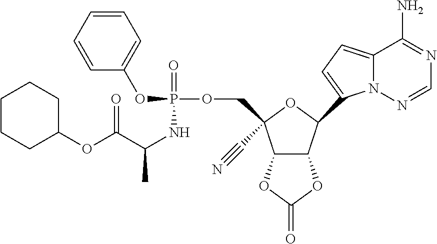 [SMILES: C[C@H](N[P@](=O)(OC[C@@]1(C#N)O[C@@H](c2ccc3c(N)ncnn23)[C@@H]2OC(=O)O[C@@H]21)Oc1ccccc1)C(=O)OC1CCCCC1]

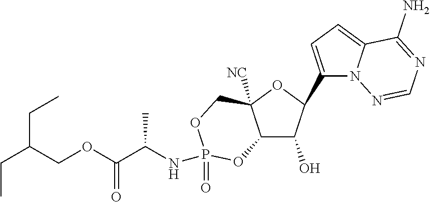

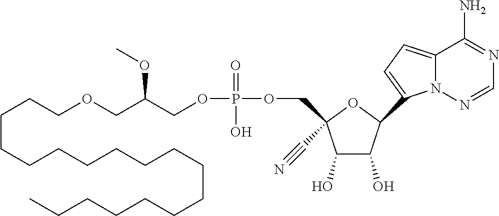 [SMILES: CCCCCCCCCCCCCCCCCCOC[C@H](COP(=O)(O)OC[C@@]1(C#N)O[C@@H](c2ccc3c(N)ncnn23)[C@H](O)[C@@H]1O)OC]

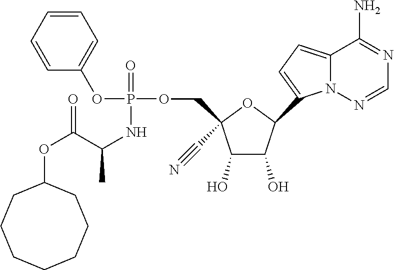 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCCCC1]

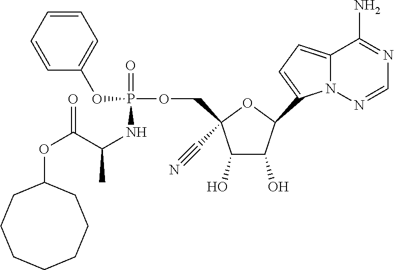 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCCCCCC1]

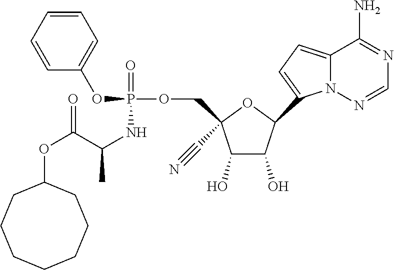 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCCCC1]

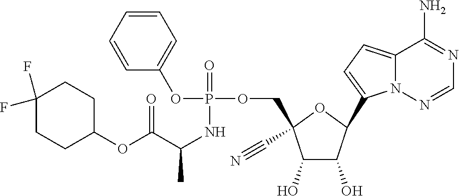 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCC(F)(F)CC1]

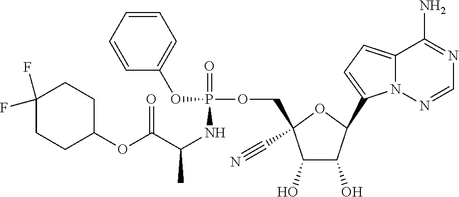 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCC(F)(F)CC1]

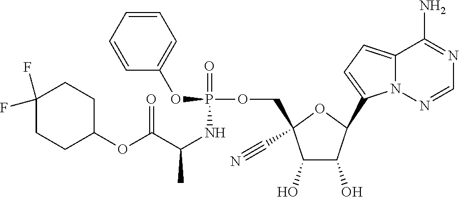 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCC(F)(F)CC1]

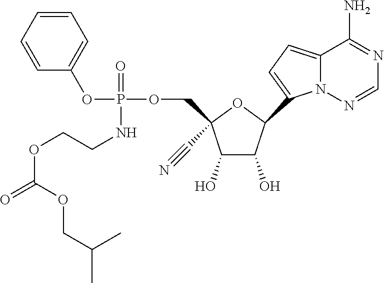 [SMILES: CC(C)COC(=O)OCCNP(=O)(OC[C@@]1(C#N)O[C@@H](c2ccc3c(N)ncnn23)[C@H](O)[C@@H]1O)Oc1ccccc1]

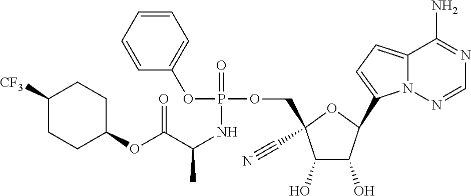 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1]

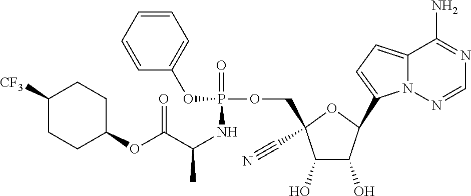 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1]

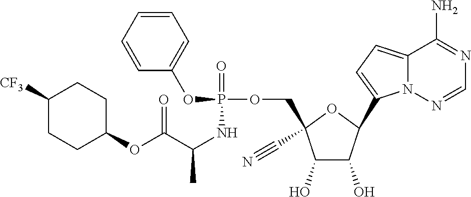 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1]

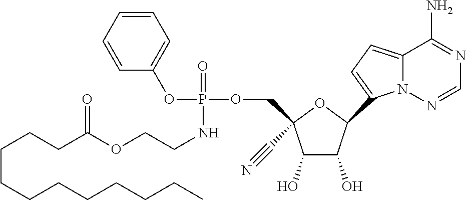 [SMILES: CCCCCCCCCCCC(=O)OCCNP(=O)(OC[C@@]1(C#N)O[C@@H](c2ccc3c(N)ncnn23)[C@H](O)[C@@H]1O)Oc1ccccc1]

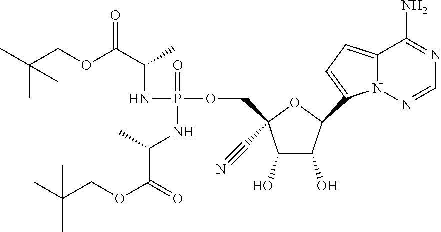 [SMILES: C[C@H](NP(=O)(N[C@@H](C)C(=O)OCC(C)(C)C)OC[C@@]1(C#N)O[C@@H](c2ccc3c(N)ncnn23)[C@H](O)[C@@H]1O)C(=O)OCC(C)(C)C]

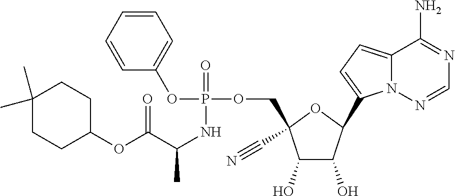 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCC(C)(C)CC1]

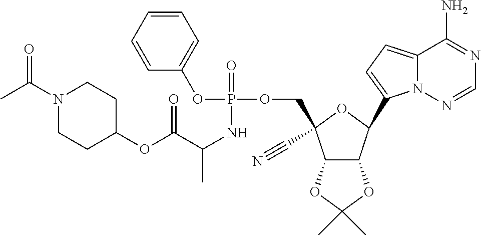 [SMILES: CC(=O)N1CCC(OC(=O)C(C)NP(=O)(OC[C@@]2(C#N)O[C@@H](c3ccc4c(N)ncnn34)[C@@H]3OC(C)(C)O[C@@H]32)Oc2ccccc2)CC1]

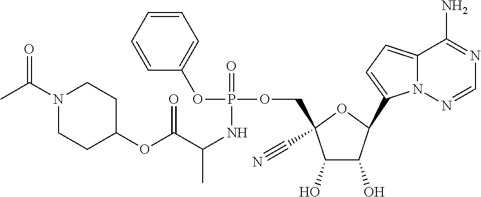 [SMILES: CC(=O)N1CCC(OC(=O)C(C)NP(=O)(OC[C@@]2(C#N)O[C@@H](c3ccc4c(N)ncnn34)[C@H](O)[C@@H]2O)Oc2ccccc2)CC1]

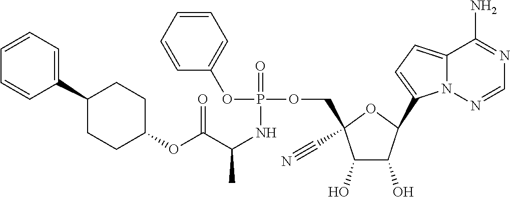 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](c2ccccc2)CC1]

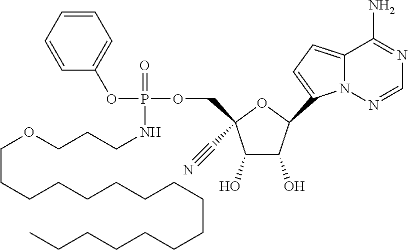 [SMILES: CCCCCCCCCCCCCCCCOCCCNP(=O)(OC[C@@]1(C#N)O[C@@H](c2ccc3c(N)ncnn23)[C@H](O)[C@@H]1O)Oc1ccccc1]

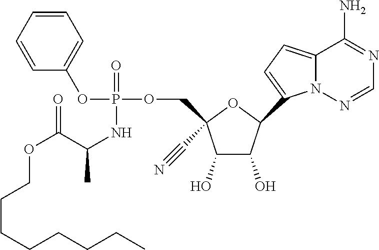 [SMILES: CCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

TABLE 1H

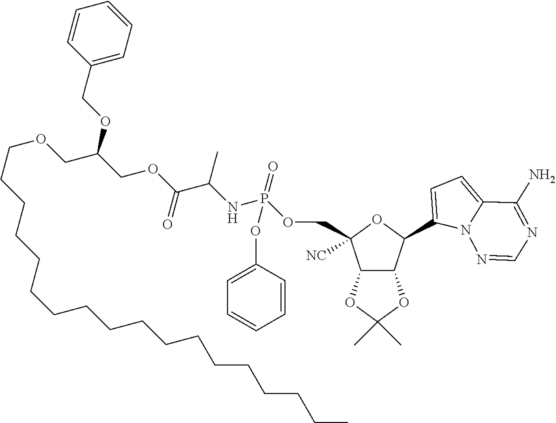 [SMILES: CCCCCCCCCCCCCCCCCCOC[C@H](COC(=O)C(C)NP(=O)(OC[C@@]1(C#N)O[C@@H](c2ccc3c(N)ncnn23)[C@@H]2OC(C)(C)O[C@@H]21)Oc1ccccc1)OCc1ccccc1]

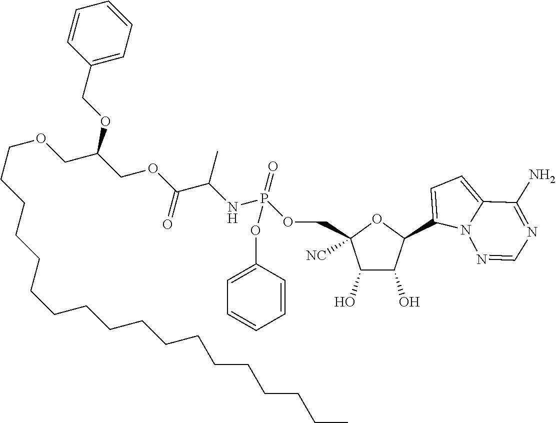 [SMILES: CCCCCCCCCCCCCCCCCCOC[C@H](COC(=O)C(C)NP(=O)(OC[C@@]1(C#N)O[C@@H](c2ccc3c(N)ncnn23)[C@H](O)[C@@H]1O)Oc1ccccc1)OCc1ccccc1]

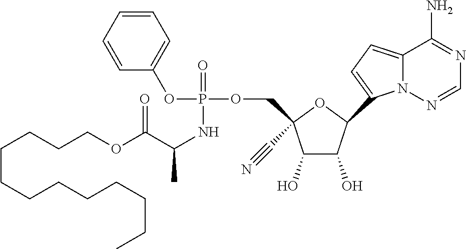 [SMILES: CCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

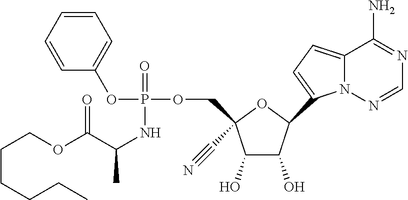 [SMILES: CCCCCCOC(=O)[C@H](C)NP(=O)(OC[C@@]1(C#N)O[C@@H](c2ccc3c(N)ncnn23)[C@H](O)[C@@H]1O)Oc1ccccc1]

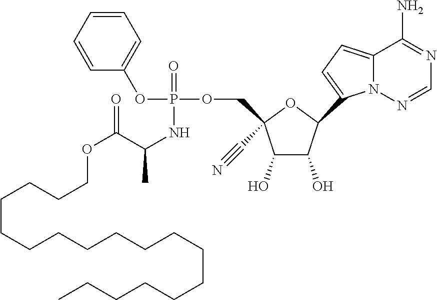 [SMILES: CCCCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

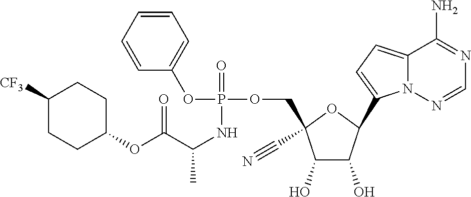 [SMILES: C[C@@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

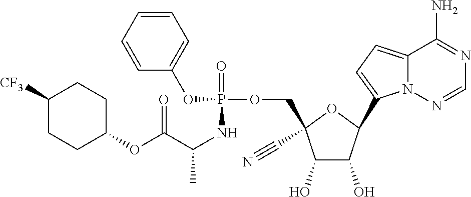 [SMILES: C[C@@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

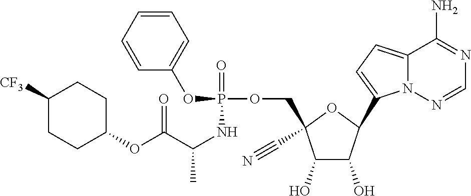 [SMILES: C[C@@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

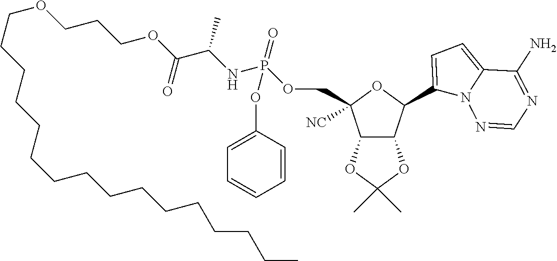 [SMILES: CCCCCCCCCCCCCCCCOCCCOC(=O)[C@H](C)NP(=O)(OC[C@@]1(C#N)O[C@@H](c2ccc3c(N)ncnn23)[C@@H]2OC(C)(C)O[C@@H]21)Oc1ccccc1]

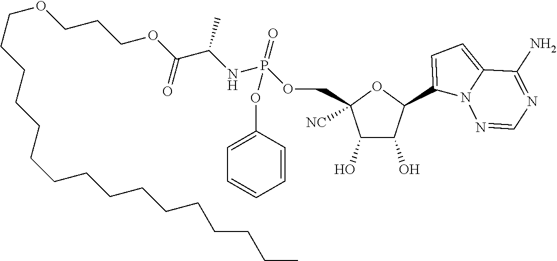 [SMILES: CCCCCCCCCCCCCCCCOCCCOC(=O)[C@H](C)NP(=O)(OC[C@@]1(C#N)O[C@@H](c2ccc3c(N)ncnn23)[C@H](O)[C@@H]1O)Oc1ccccc1]

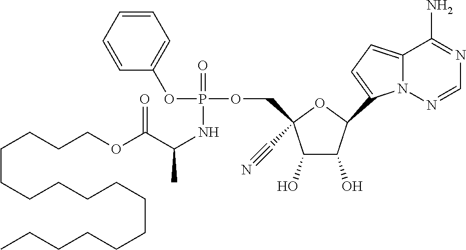 [SMILES: CCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

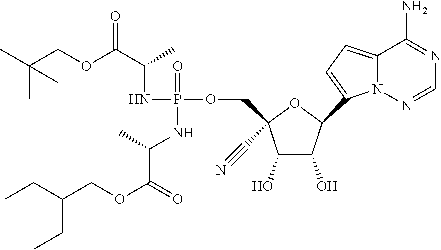 [SMILES: CCC(CC)COC(=O)[C@H](C)NP(=O)(N[C@@H](C)C(=O)OCC(C)(C)C)OC[C@@]1(C#N)O[C@@H](c2ccc3c(N)ncnn23)[C@H](O)[C@@H]1O]

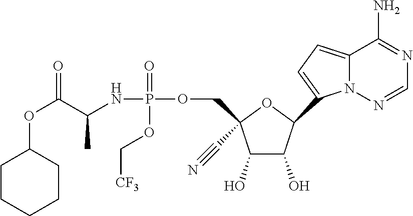 [SMILES: C[C@H](NP(=O)(OCC(F)(F)F)OC[C@@]1(C#N)O[C@@H](c2ccc3c(N)ncnn23)[C@H](O)[C@@H]1O)C(=O)OC1CCCCC1]

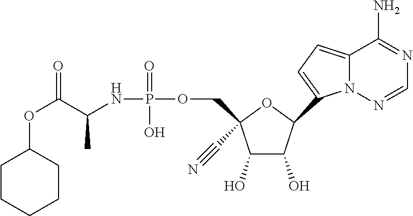 [SMILES: C[C@H](NP(=O)(O)OC[C@@]1(C#N)O[C@@H](c2ccc3c(N)ncnn23)[C@H](O)[C@@H]1O)C(=O)OC1CCCCC1]

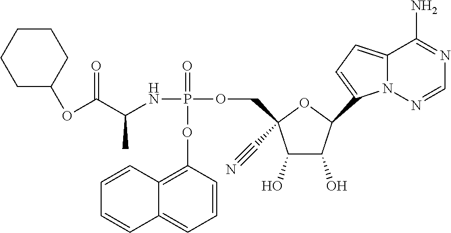 [SMILES: C[C@H](NP(=O)(OC[C@@]1(C#N)O[C@@H](c2ccc3c(N)ncnn23)[C@H](O)[C@@H]1O)Oc1cccc2ccccc12)C(=O)OC1CCCCC1]

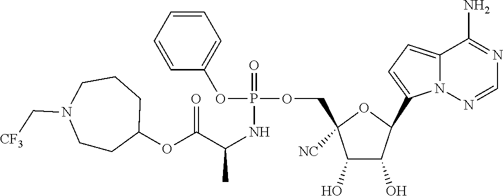 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCN(CC(F)(F)F)CC1]

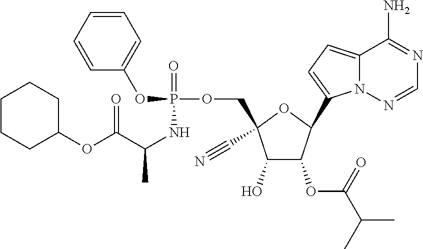 [SMILES: CC(C)C(=O)O[C@H]1[C@H](c2ccc3c(N)ncnn23)O[C@](C#N)(CO[P@@](=O)(N[C@@H](C)C(=O)OC2CCCCC2)Oc2ccccc2)[C@H]1O]

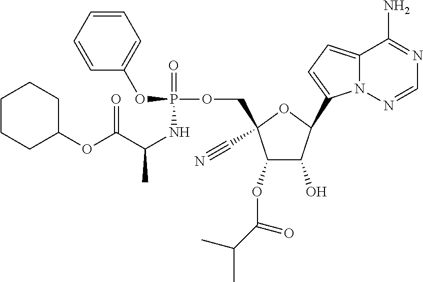 [SMILES: CC(C)C(=O)O[C@H]1[C@@H](O)[C@H](c2ccc3c(N)ncnn23)O[C@]1(C#N)CO[P@@](=O)(N[C@@H](C)C(=O)OC1CCCCC1)Oc1ccccc1]

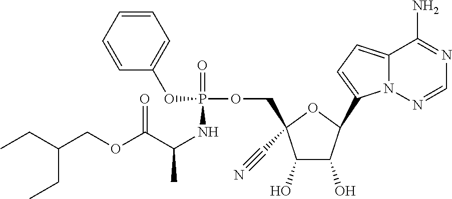 [SMILES: CCC(CC)COC(=O)[C@H](C)N[P@@](=O)(OC[C@@]1(C#N)O[C@@H](c2ccc3c(N)ncnn23)[C@H](O)[C@@H]1O)Oc1ccccc1]

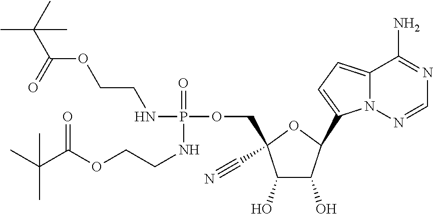 [SMILES: CC(C)(C)C(=O)OCCNP(=O)(NCCOC(=O)C(C)(C)C)OC[C@@]1(C#N)O[C@@H](c2ccc3c(N)ncnn23)[C@H](O)[C@@H]1O]

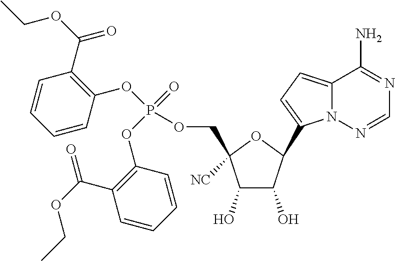 [SMILES: CCOC(=O)c1ccccc1OP(=O)(OC[C@@]1(C#N)O[C@@H](c2ccc3c(N)ncnn23)[C@H](O)[C@@H]1O)Oc1ccccc1C(=O)OCC]

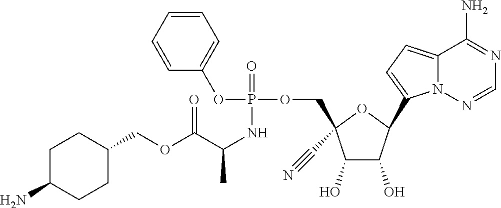 [SMILES: C[C@H](NP(=O)(OC[C@@]1(C#N)O[C@@H](c2ccc3c(N)ncnn23)[C@H](O)[C@@H]1O)Oc1ccccc1)C(=O)OC[C@H]1CC[C@H](N)CC1]

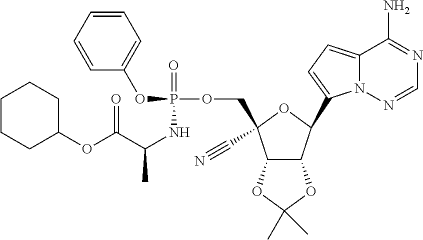 [SMILES: C[C@H](N[P@](=O)(OC[C@@]1(C#N)O[C@@H](c2ccc3c(N)ncnn23)[C@@H]2OC(C)(C)O[C@@H]21)Oc1ccccc1)C(=O)OC1CCCCC1]

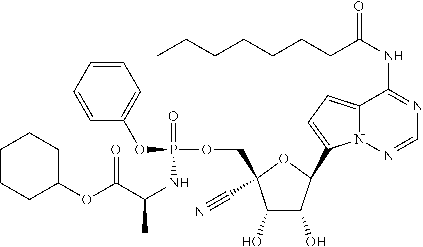 [SMILES: CCCCCCCC(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

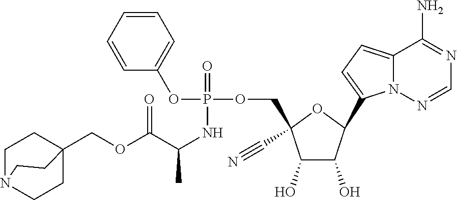 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC12CCN(CC1)CC2]

TABLE 1I

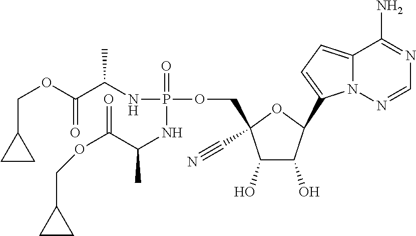 [SMILES: C[C@H](NP(=O)(N[C@@H](C)C(=O)OCC1CC1)OC[C@@]1(C#N)O[C@@H](c2ccc3c(N)ncnn23)[C@H](O)[C@@H]1O)C(=O)OCC1CC1]

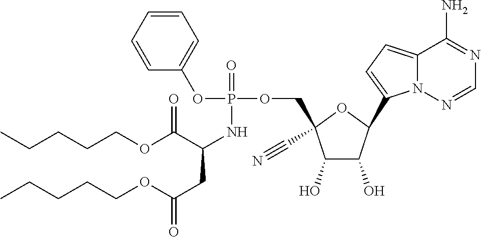 [SMILES: CCCCCOC(=O)C[C@H](NP(=O)(OC[C@@]1(C#N)O[C@@H](c2ccc3c(N)ncnn23)[C@H](O)[C@@H]1O)Oc1ccccc1)C(=O)OCCCCC]

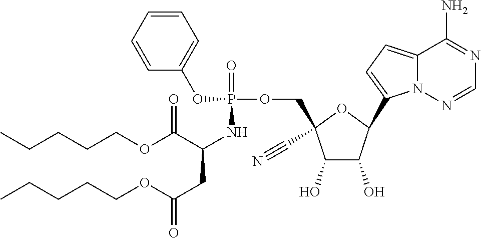 [SMILES: CCCCCOC(=O)C[C@H](N[P@@](=O)(OC[C@@]1(C#N)O[C@@H](c2ccc3c(N)ncnn23)[C@H](O)[C@@H]1O)Oc1ccccc1)C(=O)OCCCCC]

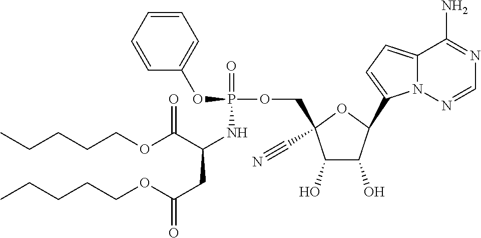 [SMILES: CCCCCOC(=O)C[C@H](N[P@](=O)(OC[C@@]1(C#N)O[C@@H](c2ccc3c(N)ncnn23)[C@H](O)[C@@H]1O)Oc1ccccc1)C(=O)OCCCCC]

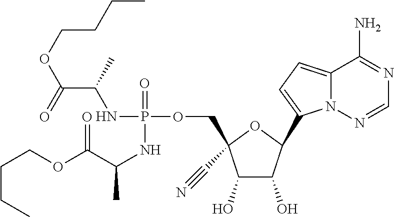 [SMILES: CCCCOC(=O)[C@H](C)NP(=O)(N[C@@H](C)C(=O)OCCCC)OC[C@@]1(C#N)O[C@@H](c2ccc3c(N)ncnn23)[C@H](O)[C@@H]1O]

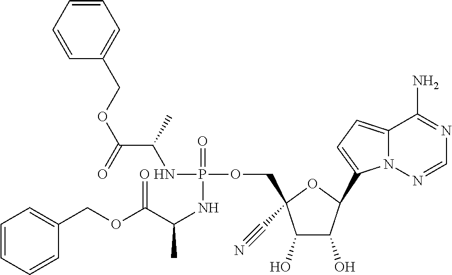 [SMILES: C[C@H](NP(=O)(N[C@@H](C)C(=O)OCc1ccccc1)OC[C@@]1(C#N)O[C@@H](c2ccc3c(N)ncnn23)[C@H](O)[C@@H]1O)C(=O)OCc1ccccc1]

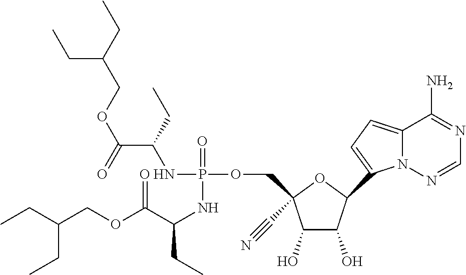 [SMILES: CCC(CC)COC(=O)[C@H](CC)NP(=O)(N[C@@H](CC)C(=O)OCC(CC)CC)OC[C@@]1(C#N)O[C@@H](c2ccc3c(N)ncnn23)[C@H](O)[C@@H]1O]

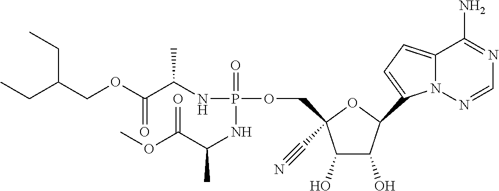 [SMILES: CCC(CC)COC(=O)[C@H](C)NP(=O)(N[C@@H](C)C(=O)OC)OC[C@@]1(C#N)O[C@@H](c2ccc3c(N)ncnn23)[C@H](O)[C@@H]1O]

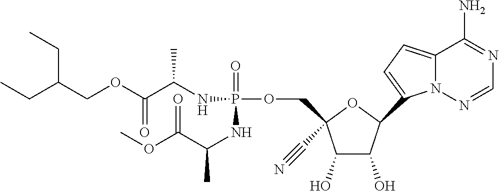 [SMILES: CCC(CC)COC(=O)[C@H](C)N[P@@](=O)(N[C@@H](C)C(=O)OC)OC[C@@]1(C#N)O[C@@H](c2ccc3c(N)ncnn23)[C@H](O)[C@@H]1O]

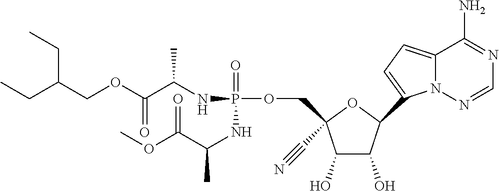 [SMILES: CCC(CC)COC(=O)[C@H](C)N[P@](=O)(N[C@@H](C)C(=O)OC)OC[C@@]1(C#N)O[C@@H](c2ccc3c(N)ncnn23)[C@H](O)[C@@H]1O]

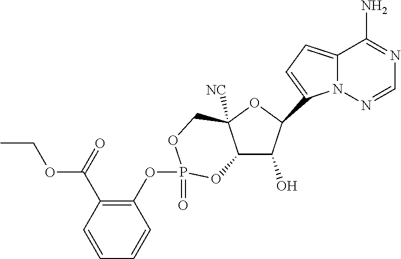

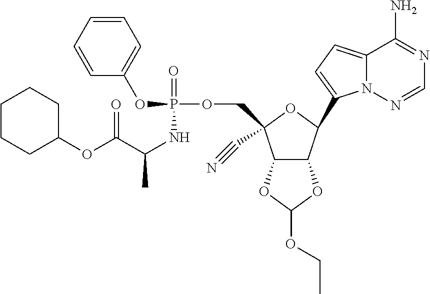

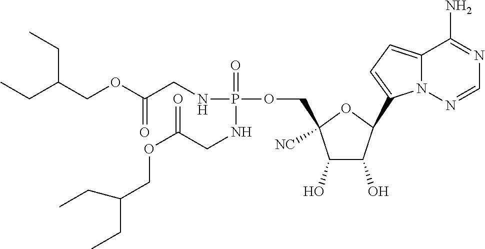 [SMILES: CCC(CC)COC(=O)CNP(=O)(NCC(=O)OCC(CC)CC)OC[C@@]1(C#N)O[C@@H](c2ccc3c(N)ncnn23)[C@H](O)[C@@H]1O]

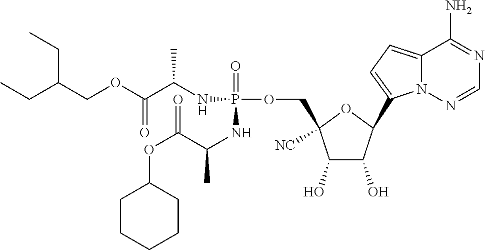 [SMILES: CCC(CC)COC(=O)[C@H](C)N[P@@](=O)(N[C@@H](C)C(=O)OC1CCCCC1)OC[C@@]1(C#N)O[C@@H](c2ccc3c(N)ncnn23)[C@H](O)[C@@H]1O]

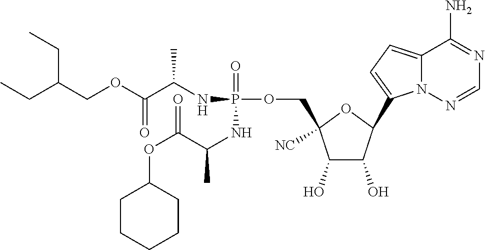 [SMILES: CCC(CC)COC(=O)[C@H](C)N[P@](=O)(N[C@@H](C)C(=O)OC1CCCCC1)OC[C@@]1(C#N)O[C@@H](c2ccc3c(N)ncnn23)[C@H](O)[C@@H]1O]

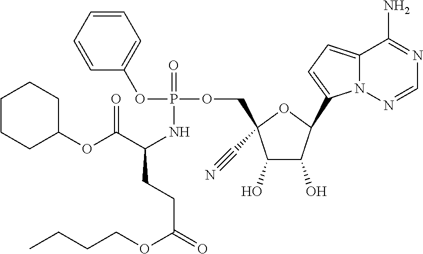 [SMILES: CCCCOC(=O)CC[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

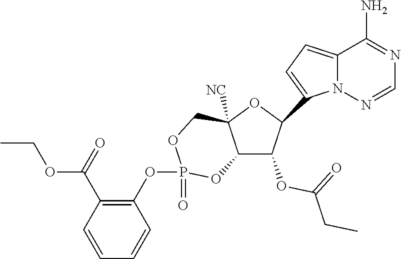

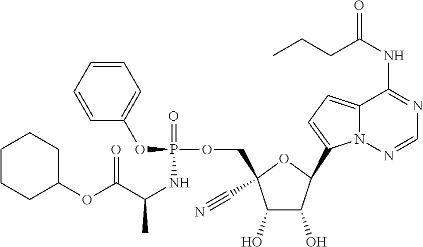 [SMILES: CCCC(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

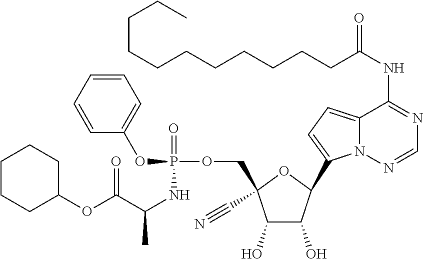 [SMILES: CCCCCCCCCCCC(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

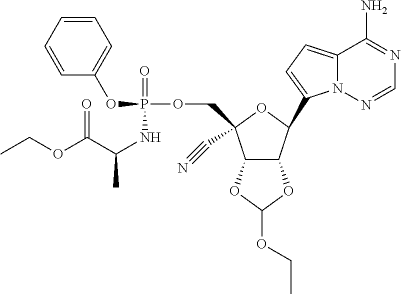

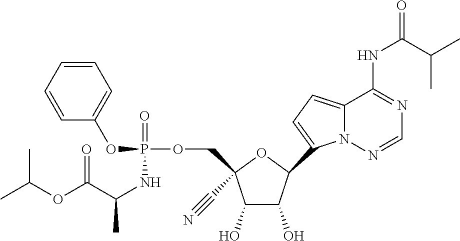 [SMILES: CC(C)OC(=O)[C@H](C)N[P@](=O)(OC[C@@]1(C#N)O[C@@H](c2ccc3c(NC(=O)C(C)C)ncnn23)[C@H](O)[C@@H]1O)Oc1ccccc1]

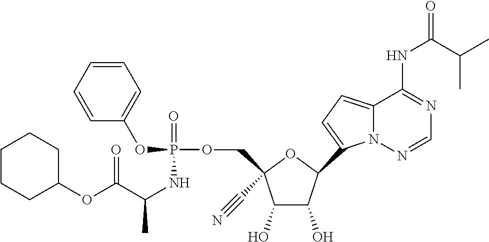 [SMILES: CC(C)C(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

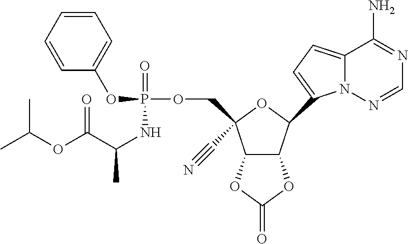 [SMILES: CC(C)OC(=O)[C@H](C)N[P@](=O)(OC[C@@]1(C#N)O[C@@H](c2ccc3c(N)ncnn23)[C@@H]2OC(=O)O[C@@H]21)Oc1ccccc1]

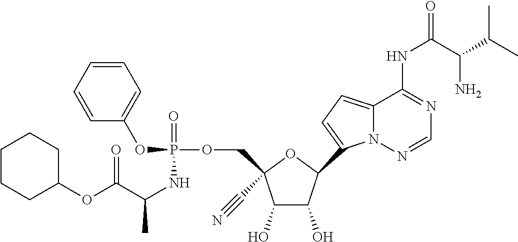 [SMILES: CC(C)[C@H](N)C(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

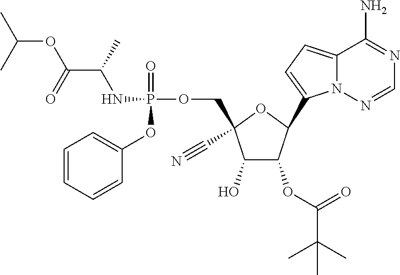 [SMILES: CC(C)OC(=O)[C@H](C)N[P@](=O)(OC[C@@]1(C#N)O[C@@H](c2ccc3c(N)ncnn23)[C@H](OC(=O)C(C)(C)C)[C@@H]1O)Oc1ccccc1]

TABLE 1J

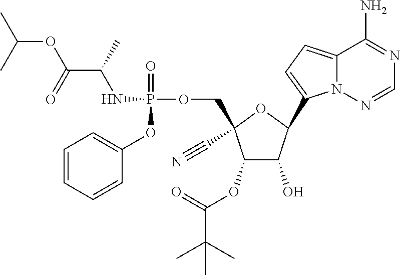 [SMILES: CC(C)OC(=O)[C@H](C)N[P@](=O)(OC[C@@]1(C#N)O[C@@H](c2ccc3c(N)ncnn23)[C@H](O)[C@@H]1OC(=O)C(C)(C)C)Oc1ccccc1]

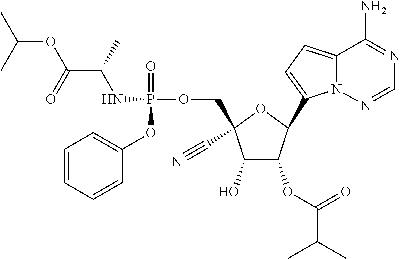 [SMILES: CC(C)OC(=O)[C@H](C)N[P@](=O)(OC[C@@]1(C#N)O[C@@H](c2ccc3c(N)ncnn23)[C@H](OC(=O)C(C)C)[C@@H]1O)Oc1ccccc1]

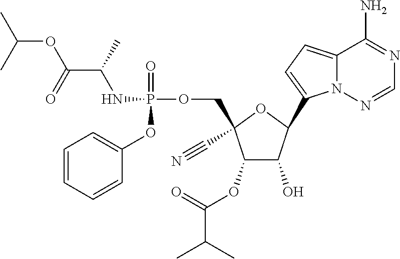 [SMILES: CC(C)OC(=O)[C@H](C)N[P@](=O)(OC[C@@]1(C#N)O[C@@H](c2ccc3c(N)ncnn23)[C@H](O)[C@@H]1OC(=O)C(C)C)Oc1ccccc1]

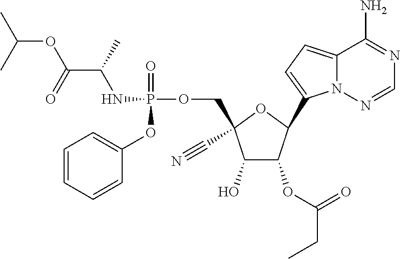 [SMILES: CCC(=O)O[C@H]1[C@H](c2ccc3c(N)ncnn23)O[C@](C#N)(CO[P@@](=O)(N[C@@H](C)C(=O)OC(C)C)Oc2ccccc2)[C@H]1O]

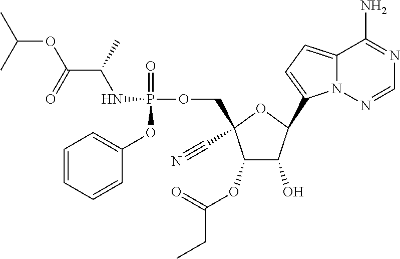 [SMILES: CCC(=O)O[C@H]1[C@@H](O)[C@H](c2ccc3c(N)ncnn23)O[C@]1(C#N)CO[P@@](=O)(N[C@@H](C)C(=O)OC(C)C)Oc1ccccc1]

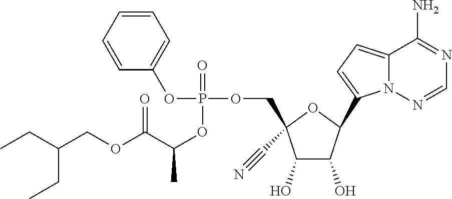 [SMILES: CCC(CC)COC(=O)[C@H](C)OP(=O)(OC[C@@]1(C#N)O[C@@H](c2ccc3c(N)ncnn23)[C@H](O)[C@@H]1O)Oc1ccccc1]

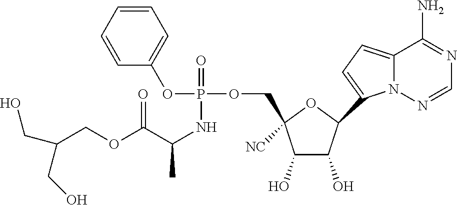 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC(CO)CO]

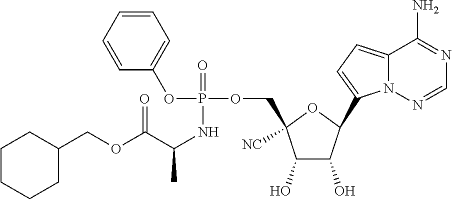 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCCCC1]

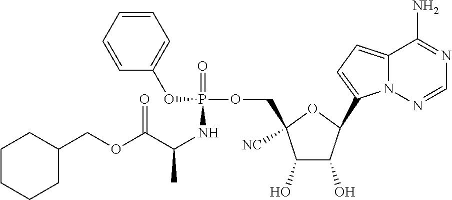 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CCCCC1]

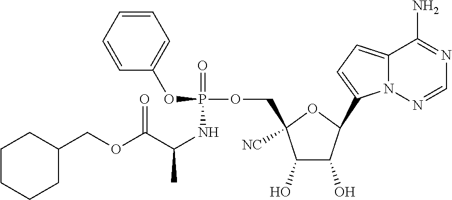 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC1CCCCC1]

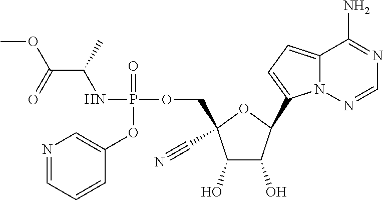 [SMILES: COC(=O)[C@H](C)NP(=O)(OC[C@@]1(C#N)O[C@@H](c2ccc3c(N)ncnn23)[C@H](O)[C@@H]1O)Oc1cccnc1]

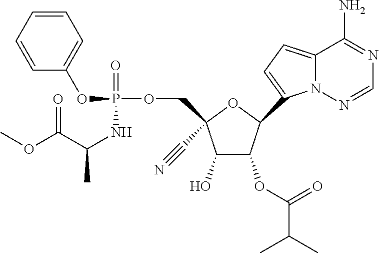 [SMILES: COC(=O)[C@H](C)N[P@](=O)(OC[C@@]1(C#N)O[C@@H](c2ccc3c(N)ncnn23)[C@H](OC(=O)C(C)C)[C@@H]1O)Oc1ccccc1]

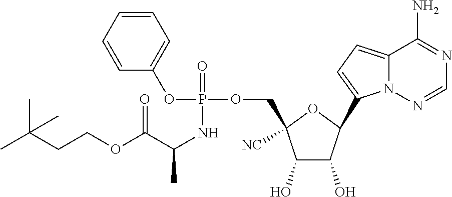 [SMILES: C[C@H](NP(=O)(OC[C@@]1(C#N)O[C@@H](c2ccc3c(N)ncnn23)[C@H](O)[C@@H]1O)Oc1ccccc1)C(=O)OCCC(C)(C)C]

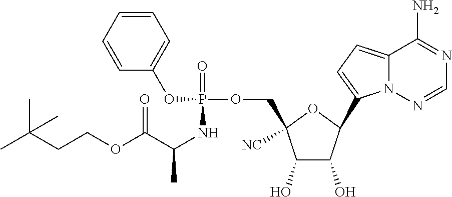 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCCC(C)(C)C]

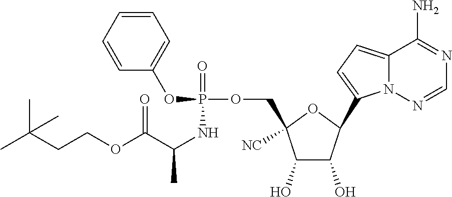 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCCC(C)(C)C]

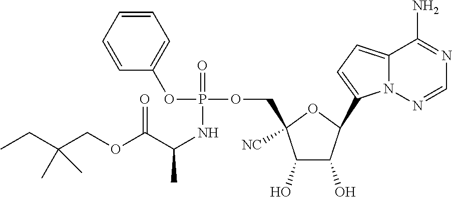 [SMILES: CCC(C)(C)COC(=O)[C@H](C)NP(=O)(OC[C@@]1(C#N)O[C@@H](c2ccc3c(N)ncnn23)[C@H](O)[C@@H]1O)Oc1ccccc1]

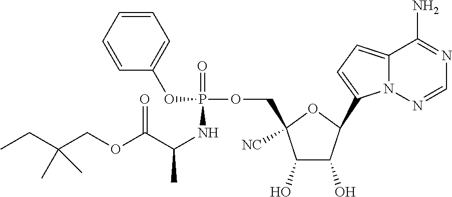 [SMILES: CCC(C)(C)COC(=O)[C@H](C)N[P@@](=O)(OC[C@@]1(C#N)O[C@@H](c2ccc3c(N)ncnn23)[C@H](O)[C@@H]1O)Oc1ccccc1]

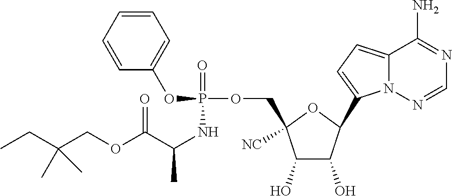 [SMILES: CCC(C)(C)COC(=O)[C@H](C)N[P@](=O)(OC[C@@]1(C#N)O[C@@H](c2ccc3c(N)ncnn23)[C@H](O)[C@@H]1O)Oc1ccccc1]

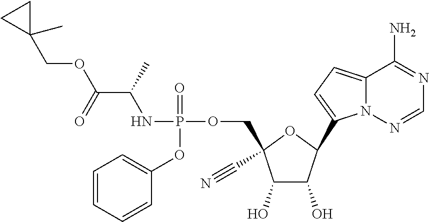 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1(C)CC1]

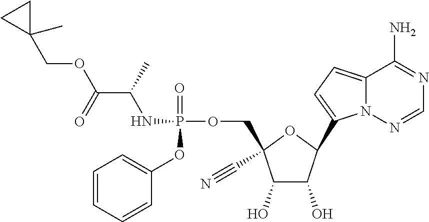 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC1(C)CC1]

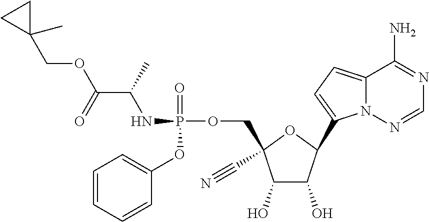 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1(C)CC1]

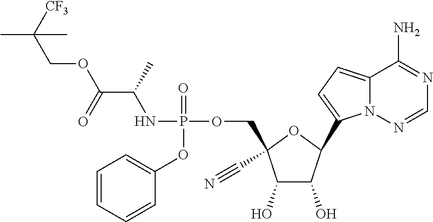 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC(C)(C)C(F)(F)F]

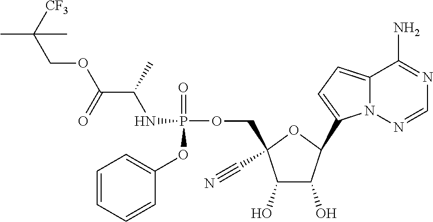 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC(C)(C)C(F)(F)F]

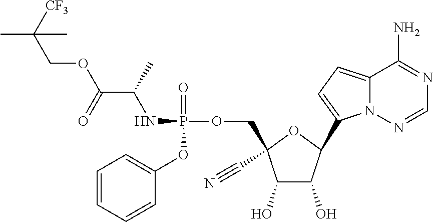 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC(C)(C)C(F)(F)F]

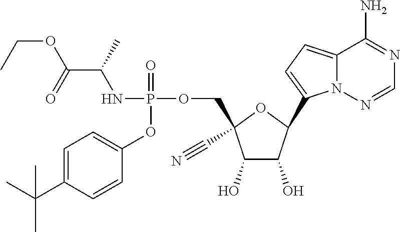 [SMILES: CCOC(=O)[C@H](C)NP(=O)(OC[C@@]1(C#N)O[C@@H](c2ccc3c(N)ncnn23)[C@H](O)[C@@H]1O)Oc1ccc(C(C)(C)C)cc1]

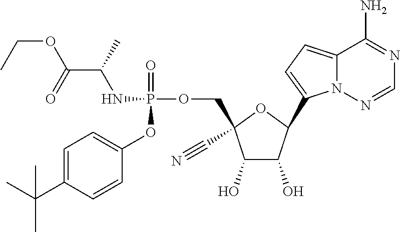 [SMILES: CCOC(=O)[C@H](C)N[P@](=O)(OC[C@@]1(C#N)O[C@@H](c2ccc3c(N)ncnn23)[C@H](O)[C@@H]1O)Oc1ccc(C(C)(C)C)cc1]

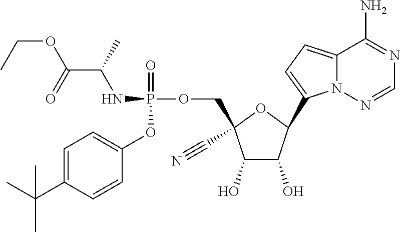 [SMILES: CCOC(=O)[C@H](C)N[P@@](=O)(OC[C@@]1(C#N)O[C@@H](c2ccc3c(N)ncnn23)[C@H](O)[C@@H]1O)Oc1ccc(C(C)(C)C)cc1]

In some embodiments, the present disclosure provides a compound, or a pharmaceutically acceptable salt thereof, having the structure of:

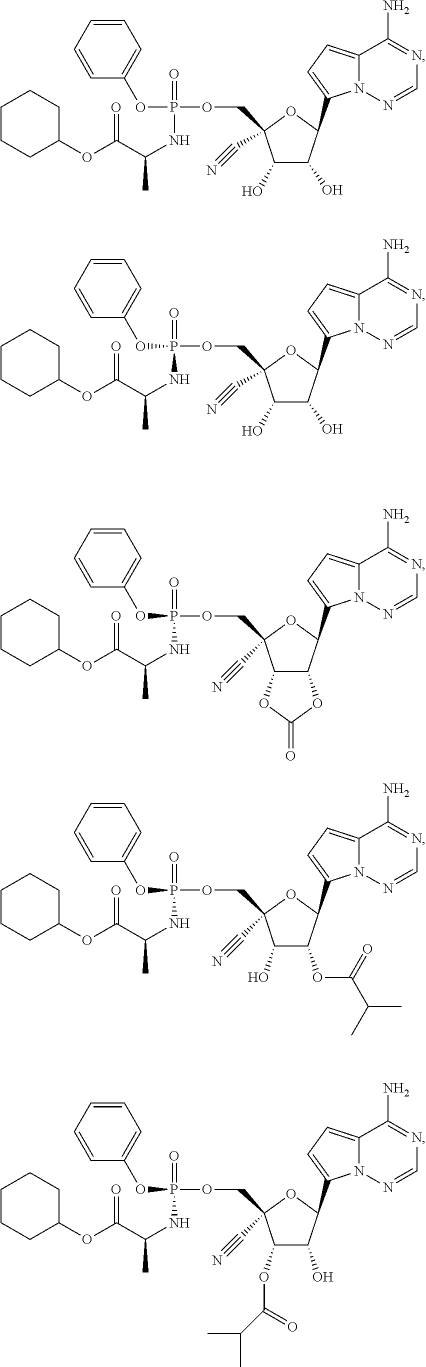

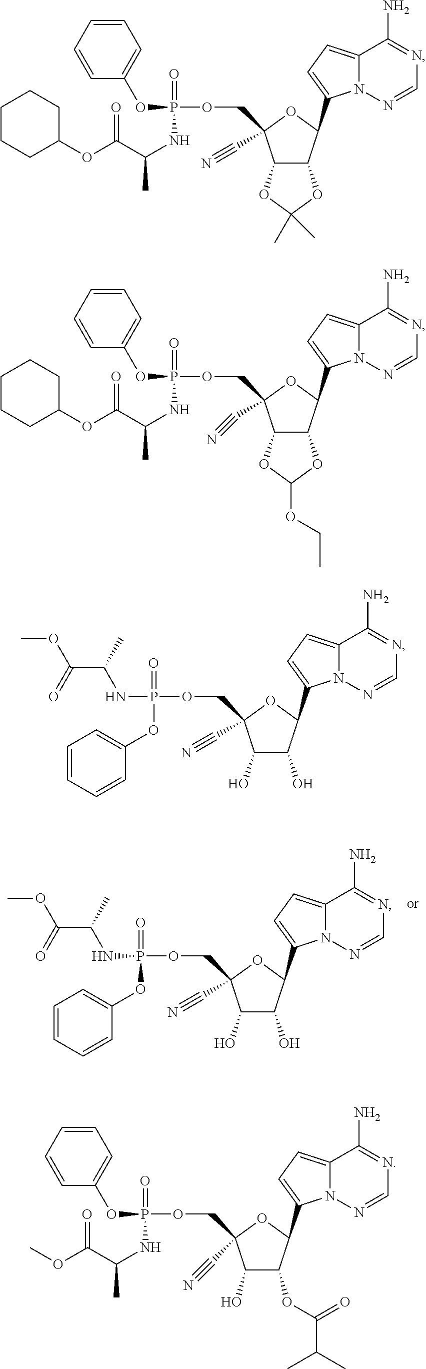

In some embodiments, the present disclosure provides a compound, or a pharmaceutically acceptable salt thereof, having the structure of:

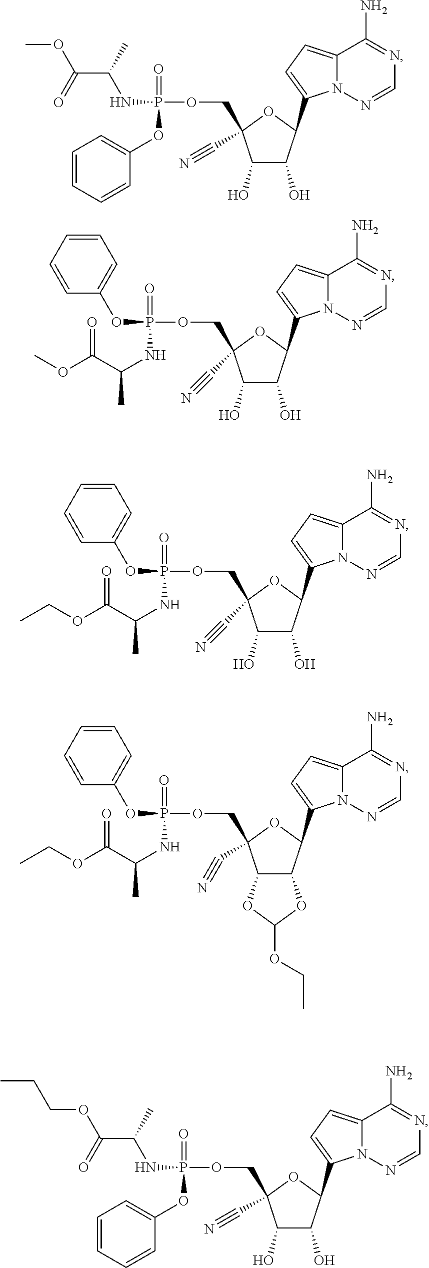

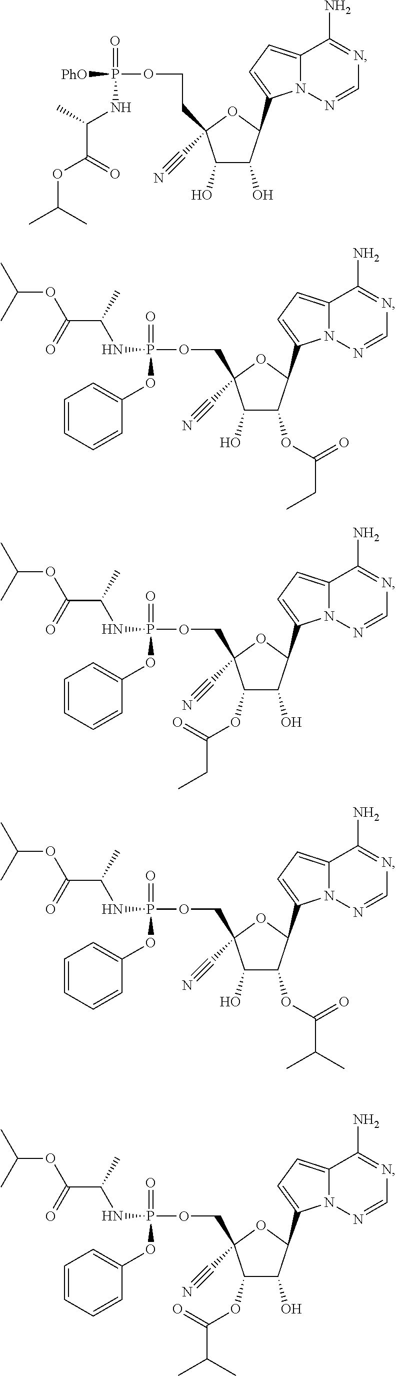

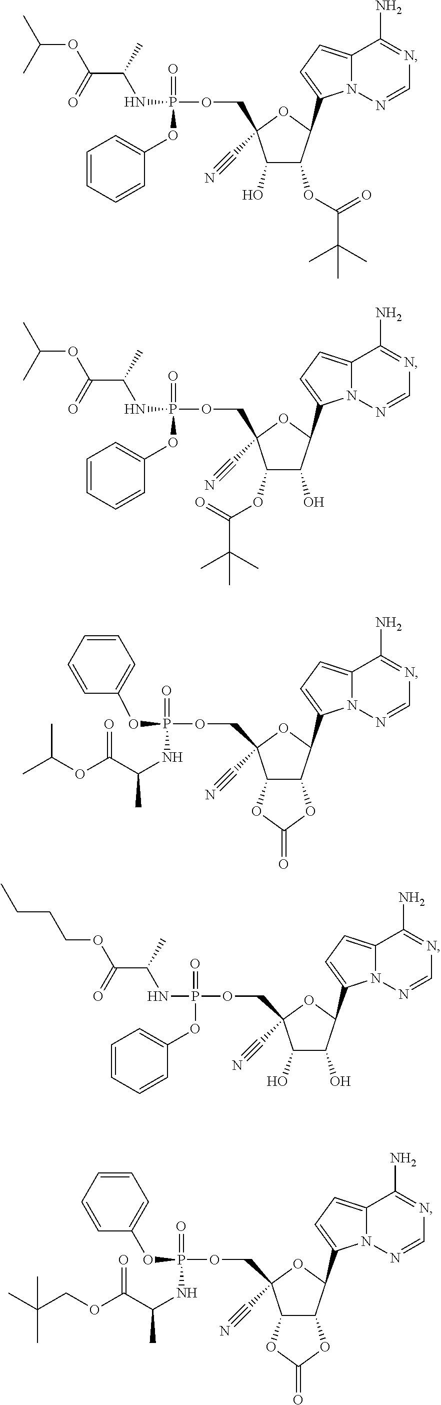

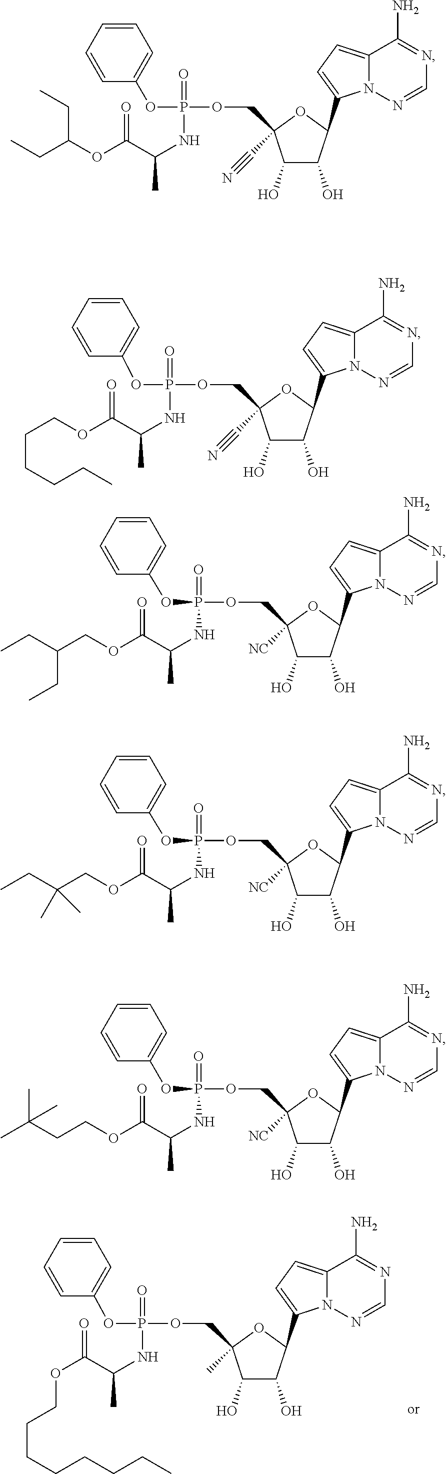

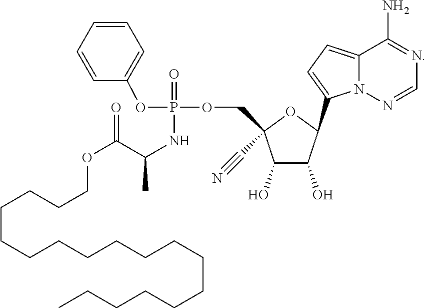 [SMILES: CCCCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

In some embodiments, with the proviso that when the compound of Formula (Ia), (Ib), (Ic), (Id), (Ie), (If), (Ig), (Ih) or (Ii) has the formula:

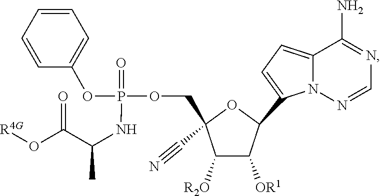

and R^{4G} is ethyl or 2-ethylbutyl, then one of R¹ and R² is —C(O)R^{1A}, or R¹ and R² are combined to form —C(O)— or —C(R^{2A})(R^{2B})—. In some embodiments, the compound can be represented by Formula (Ia), (Ib), (Ic), (Id), (Ie), (If), (Ig), (Ih) or (Ii), or a pharmaceutically acceptable salt thereof, with the proviso that the compound is not:

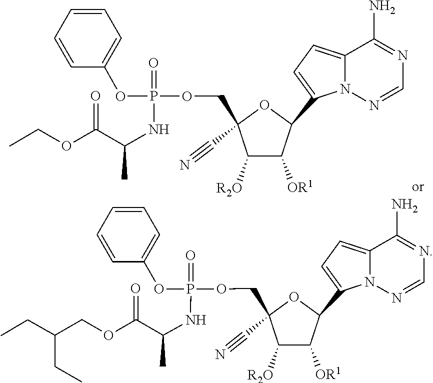

Also falling within the scope herein are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g. ¹⁴C or ³H) compound, administering it parenterally in a detectable dose (e.g. greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g. by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

### IV. Pharmaceutical Formulations

In some embodiments, the present disclosure provides a pharmaceutical formulation comprising a pharmaceutically effective amount of a compound of the present disclosure, or a pharmaceutically acceptable salt thereof, and a pharmaceutically acceptable carrier or excipient. Also provided herein is a pharmaceutical formulation comprising a pharmaceutically effective amount of a compound of Formula (Ia), (Ib), (Ic), (Id), (Ie), (If), (Ig), (Ih) and (Ii), or a pharmaceutically acceptable salt, solvate, and/or ester thereof, and a pharmaceutically acceptable carrier or excipient.

The compounds herein are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, comprise at least one active ingredient, as above defined, together with one or more acceptable carriers and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations herein comprise a combination together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, solutions, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions may be in the form of a sterile injectable or intravenous preparations, such as a sterile injectable aqueous or oleaginous suspension. This suspension may be formulated according to the known art using those suitable dispersing or wetting agents and suspending agents which have been mentioned above. The sterile injectable or intravenous preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of Pneumoviridae infections as described below.

Another embodiments provides a novel, efficacious, safe, nonirritating and physiologically compatible inhalable composition comprising a compound of Formula (Ia), (Ib), (Ic), (Id), (Ie), (If), (Ig), (Ih) or (Ii), or a pharmaceutically acceptable salt thereof, suitable for treating Pneumoviridae infections and potentially associated bronchiolitis. Preferred pharmaceutically acceptable salts are inorganic acid salts including hydrochloride, hydrobromide, sulfate or phosphate salts as they may cause less pulmonary irritation. Preferably, the inhalable formulation is delivered to the endobronchial space in an aerosol comprising particles with a mass median aerodynamic diameter (MMAD) between about 1 and about 5 μm. Preferably, the compound of Formula (Ia), (Ib), (Ic), (Id), (Ie), (If), (Ig), (Ih) or (Ii) is formulated for aerosol delivery using a nebulizer, pressurized metered dose inhaler (pMDI), or dry powder inhaler (DPI).

Non-limiting examples of nebulizers include atomizing, jet, ultrasonic, pressurized, vibrating porous plate, or equivalent nebulizers including those nebulizers utilizing adaptive aerosol delivery technology (Denyer, J. Aerosol medicine Pulmonary Drug Delivery 2010, 23 Supp 1, S1-S10). A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes.

In a preferred embodiment, the formulation for nebulization is delivered to the endobronchial space in an aerosol comprising particles with a MMAD predominantly between about 1 μm and about 5 μm using a nebulizer able to aerosolize the formulation of the compound of Formula (Ia), (Ib), (Ic), (Id), (Ie), (If), (Ig), (Ih) or (Ii) into particles of the required MMAD. To be optimally therapeutically effective and to avoid upper respiratory and systemic side effects, the majority of aerosolized particles should not have a MMAD greater than about 5 μm. If an aerosol contains a large number of particles with a MMAD larger than 5 μm, the particles are deposited in the upper airways decreasing the amount of drug delivered to the site of inflammation and bronchoconstriction in the lower respiratory tract. If the MMAD of the aerosol is smaller than about 1 μm, then the particles have a tendency to remain suspended in the inhaled air and are subsequently exhaled during expiration.

When formulated and delivered according to the method herein, the aerosol formulation for nebulization delivers a therapeutically efficacious dose of the compound of Formula (Ia), (Ib), (Ic), (Id), (Ie), (If), (Ig), (Ih) or (Ii) to the site of Pneumoviridae infection sufficient to treat the Pneumoviridae infection. The amount of drug administered must be adjusted to reflect the efficiency of the delivery of a therapeutically efficacious dose of the compound of Formula (Ia), (Ib), (Ic), (Id), (Ie), (If), (Ig), (Ih) or (Ii). In a preferred embodiment, a combination of the aqueous aerosol formulation with the atomizing, jet, pressurized, vibrating porous plate, or ultrasonic nebulizer permits, depending on the nebulizer, about, at least, 20, to about 90%, typically about 70% delivery of the administered dose of the compound of Formula (Ia), (Ib), (Ic), (Id), (Ie), (If), (Ig), (Ih) or (Ii) into the airways. In a preferred embodiment, at least about 30 to about 50% of the active compound is delivered. More preferably, about 70 to about 90% of the active compound is delivered.

In another embodiment, a compound of Formula (Ia), (Ib), (Ic), (Id), (Ie), (If), (Ig), (Ih) or (Ii) or a pharmaceutically acceptable salt thereof, is delivered as a dry inhalable powder. The compounds are administered endobronchially as a dry powder formulation to efficacious deliver fine particles of compound into the endobronchial space using dry powder or metered dose inhalers. For delivery by DPI, the compound of Formula (Ia), (Ib), (Ic), (Id), (Ie), (If), (Ig), (Ih) or (Ii) is processed into particles with, predominantly, MMAD between about 1 μm and about 5 μm by milling spray drying, critical fluid processing, or precipitation from solution. Media milling, jet milling and spray-drying devices and procedures capable of producing the particle sizes with a MMAD between about 1 μm and about 5 μm are well known in the art. In one embodiment, excipients are added to the compound of Formula (Ia), (Ib), (Ic), (Id), (Ie), (If), (Ig), (Ih) or (Ii) before processing into particles of the required sizes. In another embodiment, excipients are blended with the particles of the required size to aid in dispersion of the drug particles, for example by using lactose as an excipient.

Particle size determinations are made using devices well known in the art. For example a multi-stage Anderson cascade impactor or other suitable method such as those specifically cited within the US Pharmacopoeia Chapter 601 as characterizing devices for aerosols within metered-dose and dry powder inhalers.

In another preferred embodiment, a compound of Formula (Ia), (Ib), (Ic), (Id), (Ie), (If), (Ig), (Ih) or (Ii) is delivered as a dry powder using a device such as a dry powder inhaler or other dry powder dispersion devices. Non-limiting examples of dry powder inhalers and devices include those disclosed in U.S. Pat. Nos. 5,458,135; 5,740,794; 5,775,320; 5,785,049; 3,906,950; 4,013,075; 4,069,819; 4,995,385; 5,522,385; 4,668,218; 4,667,668; 4,805,811 and 5,388,572. There are two major designs of dry powder inhalers. One design is a metering device in which a reservoir for the drug is place within the device and the patient adds a dose of the drug into the inhalation chamber. The second design is a factory-metered device in which each individual dose has been manufactured in a separate container. Both systems depend on the formulation of the drug into small particles of MMAD from 1 μm and about 5 μm and often involve co-formulation with larger excipient particles such as, but not limited to, lactose. Drug powder is placed in the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to decompose, and the mass of the large excipient particles causes their impaction at the back of the throat, while the smaller drug particles are deposited deep in the lungs. In preferred embodiments, a compound of Formula (Ia), (Ib), (Ic), (Id), (Ie), (If), (Ig), (Ih) or (Ii), or a pharmaceutically acceptable salt thereof, is delivered as a dry powder using either type of dry powder inhaler as described herein, wherein the MMAD of the dry powder, exclusive of any excipients, is predominantly in the range of 1 μm to about 5 μm.

In another embodiment, a compound of Formula (Ia), (Ib), (Ic), (Id), (Ie), (If), (Ig), (Ih) or (Ii) is delivered as a dry powder using a metered dose inhaler. Non-limiting examples of metered dose inhalers and devices include those disclosed in U.S. Pat. Nos. 5,261,538; 5,544,647; 5,622,163; 4,955,371; 3,565,070; 3,361,306 and 6,116,234. In preferred embodiments, a compound of Formula (Ia), (Ib), (Ic), (Id), (Ie), (If), (Ig), (Ih) or (Ii), or a pharmaceutically acceptable salt thereof, is delivered as a dry powder using a metered dose inhaler wherein the MMAD of the dry powder, exclusive of any excipients, is predominantly in the range of about 1-5 μm.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations may include other agents conventional in the art having regard to the type of formulation in question, for example those suitable for oral administration may include flavoring agents.

Further provided are veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds herein are used to provide controlled release pharmaceutical formulations containing as active ingredient one or more of the compounds (“controlled release formulations”) in which the release of the active ingredient is controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### V. Routes of Administration

One or more of the compounds of Formula (Ia), (Ib), (Ic), (Id), (Ie), (If), (Ig), (Ih) or (Ii) (herein referred to as the active ingredients) are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the preferred route may vary with for example the condition of the recipient.

The compounds of the present disclosure (also referred to herein as the active ingredients), can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient. An advantage of certain compounds disclosed herein is that they are orally bioavailable and can be dosed orally.

A compound of the present disclosure, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In certain embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from 1 mg to 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, 125, 150, 175, 200, 225, 250, 275, 300, 325, 350, 375, 400, 425, 450, 475, or about 500 mg per dose. Other therapeutically effective amounts of the compound of the present disclosure are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month.

Other therapeutically effective amounts of the compound of the present disclosure are about 20, 25, 30, 35, 40, 45, 50, 55, 60, 65, 70, 75, 80, 85, 90, 95, or about 100 mg per dose.

The frequency of dosage of the compound of the present disclosure are will be determined by the needs of the individual patient and can be, for example, once per day or twice, or more times, per day. Administration of the compound continues for as long as necessary to treat the viral infection. For example, a compound can be administered to a human being infected with a virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

In one embodiment, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In one embodiment, kits comprising a compound of the present disclosure, or a pharmaceutically acceptable salt thereof, in combination with one or more (e.g., one, two, three, four, one or two, one to three, or one to four) additional therapeutic agents are provided.

In certain embodiments, a compound of the present disclosure, or a pharmaceutically acceptable salt thereof, is combined with one, two, three, four or more additional therapeutic agents. In certain embodiments, a compound of the present disclosure, or a pharmaceutically acceptable salt thereof, is combined with two additional therapeutic agents. In other embodiments, a compound of the present disclosure, or a pharmaceutically acceptable salt thereof, is combined with three additional therapeutic agents. In further embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In certain embodiments, when a compound of the present disclosure is combined with one or more additional therapeutic agents as described herein, the components of the composition are administered as a simultaneous or sequential regimen. When administered sequentially, the combination may be administered in two or more administrations.

In certain embodiments, a compound of the present disclosure is combined with one or more additional therapeutic agents in a unitary dosage form for simultaneous administration to a patient, for example as a solid dosage form for oral administration.

In certain embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping a compound in liposomes or microemulsions that are compatible with body tissues.

### VI. Combination Therapy

The compounds of Formula (Ia), (Ib), (Ic), (Id), (Ie), (If), (Ig), (Ih) or (Ii) and compositions provided herein are also used in combination with other active therapeutic agents for the treatment of virus infections, such as Pneumoviridae, Picornaviridae, Flaviviridae, or Filoviridae virus infections.

### Combination Therapy for the Treatment of Pneumoviridae

The compounds and compositions provided herein are also used in combination with other active therapeutic agents. For the treatment of Pneumoviridae virus infections, preferably, the other active therapeutic agent is active against Pneumoviridae virus infections, particularly respiratory syncytial virus infections and/or metapneumovirus infections. Non-limiting examples of these other active therapeutic agents active against RSV are ribavirin, palivizumab, motavizumab, RSV-IGIV (RespiGam®), MEDI-557, A-60444 (also known as RSV604), MDT-637, BMS-433771, ALN-RSV0, ALX-0171 and mixtures thereof. Other non-limiting examples of other active therapeutic agents active against respiratory syncytial virus infections include respiratory syncytial virus protein F inhibitors, such as AK-0529; RV-521, ALX-0171, JNJ-53718678, BTA-585, and presatovir; RNA polymerase inhibitors, such as lumicitabine and ALS-8112; anti-RSV G protein antibodies, such as anti-G-protein mAb; viral replication inhibitors, such as nitazoxanide.

In some embodiments, the other active therapeutic agent may be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGP084-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent may be a vaccine for the treatment or prevention of metapneumovirus infections, including but not limited to mRNA-1653 and rHMPV-Pa vaccine.

### Combination Therapy for the Treatment of Picornaviridae

The compounds and compositions provided herein are also used in combination with other active therapeutic agents. For the treatment of Picornaviridae virus infections, preferably, the other active therapeutic agent is active against Picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody;

In some embodiments, the other active therapeutic agent may be a vaccine for the treatment or prevention of Picornaviridae virus infections, including but not limited to EV71 vaccines, TAK-021, and EV-D68 adenovector-based vaccine.

### Combination Therapy for Respiratory Infections

Many of the infections of the Pneumoviridae and Picornaviridae viruses are respiratory infections. Therefore, additional active therapeutics used to treat respiratory symptoms and sequelae of infection may be used in combination with the compounds provided herein. The additional agents are preferably administered orally or by direct inhalation. For example, other preferred additional therapeutic agents in combination with the compounds provided herein for the treatment of viral respiratory infections include, but are not limited to, bronchodilators and corticosteroids.

Glucocorticoids

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the compounds provided herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

Anti-Inflammatory Agents

Other anti-inflammatory agents working through anti-inflammatory cascade mechanisms are also useful as additional therapeutic agents in combination with the compounds provided herein for the treatment of viral respiratory infections. Applying “anti-inflammatory signal transduction modulators” (referred to in this text as AISTM), like phosphodiesterase inhibitors (e.g. PDE-4, PDE-5, or PDE-7 specific), transcription factor inhibitors (e.g. blocking NFxB through IKK inhibition), or kinase inhibitors (e.g. blocking P38 MAP, JNK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluoromethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

β2-Adrenoreceptor Agonist Bronchodilators

Combinations comprising inhaled β2-adrenoreceptor agonist bronchodilators such as formoterol, albuterol or salmeterol with the compounds provided herein are also suitable, but non-limiting, combinations useful for the treatment of respiratory viral infections.

Combinations of inhaled β2-adrenoreceptor agonist bronchodilators such as formoterol or salmeterol with ICS's are also used to treat both the bronchoconstriction and the inflammation (Symbicort® and Advair®, respectively). The combinations comprising these ICS and β2-adrenoreceptor agonist combinations along with the compounds provided herein are also suitable, but non-limiting, combinations useful for the treatment of respiratory viral infections.

Other examples of Beta 2 adrenoceptor agonists are bedoradrine, vilanterol, indacaterol, olodaterol, tulobuterol, formoterol, abediterol, salbutamol, arformoterol, levalbuterol, fenoterol, and TD-5471.

Anticholinergics

For the treatment or prophylaxis of pulmonary broncho-constriction, anticholinergics are of potential use and, therefore, useful as an additional therapeutic agent in combination with the compounds provided herein for the treatment of viral respiratory infections. These anticholinergics include, but are not limited to, antagonists of the muscarinic receptor (particularly of the M3 subtype) which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, bencycloquidium bromide.

Mucolytic Agents

The compounds provided herein and the compositions provided herein may also be combined with mucolytic agents to treat both the infection and symptoms of respiratory infections. A non-limiting example of a mucolytic agent is ambroxol. Similarly, the compounds of Formula (I), (Ia), (Ib), (Ic), (Id), (Ie), (If), (Ig), (Ih), or (Ii) may be combined with expectorants to treat both the infection and symptoms of respiratory infections. A non-limiting example of an expectorant is guaifenesin.

Nebulized hypertonic saline is used to improve immediate and long-term clearance of small airways in patients with lung diseases (Kuzik, J. Pediatrics 2007, 266). Thus, the compounds provided herein may also be combined with nebulized hypertonic saline particularly when the Pneumoviridae virus infection is complicated with bronchiolitis. The combination of the compounds of Formula (I) or Formula (II) with hypertonic saline may also comprise any of the additional agents discussed above. In one embodiment, nebulized about 3% hypertonic saline is used.

### Combination Therapy for the Treatment of COPD

The compounds and compositions provided herein are also used in combination with other active therapeutic agents. For the treatment of respiratory exacerbations of COPD, the other active therapeutic agents include other active against COPD. Non-limiting examples of these other active therapeutic agents include anti-IL5 antibodies, such as benralizumab, mepolizumab; dipeptidyl peptidase I (DPP1) inhibitors, such as AZD-7986 (INS-1007); DNA gyrase inhibitor/topoisomerase IV inhibitors, such as ciprofloxacin hydrochloride; MDR associated protein 4/phosphodiesterase (PDE) 3 and 4 inhibitors, such as RPL-554; CFTR stimulators, such as ivacaftor, QBW-251; MMP-9/MMP-12 inhibitors, such as RBx-10017609; Adenosine A1 receptor antagonists, such as PBF-680; GATA 3 transcription factor inhibitors, such as SB-010; muscarinic receptor modulator/nicotinic acetylcholine receptor agonists, such as ASM-024; MARCKS protein inhibitors, such as BIO-11006; kit tyrosine kinase/PDGF inhibitors such as masitinib; phosphodiesterase (PDE) 4 inhibitors, such as roflumilast, CHF-6001; phosphoinositide-3 kinase delta inhibitors, such as nemiralisib; 5-Lipoxygenase inhibitors, such as TA-270; muscarinic receptor antagonist/beta 2 adrenoceptor agonist, such as batefenterol succinate, AZD-887, ipratropium bromide; TRN-157; elastase inhibitors, such as erdosteine; metalloprotease-12 inhibitors such as FP-025; interleukin 18 ligand inhibitors, such as tadekinig alfa; skeletal muscle troponin activators, such as CK-2127107; p38 MAP kinase inhibitors, such as acumapimod; IL-17 receptor modulators, such as CNTO-6785; CXCR2 chemokine antagonists, such as danirixin; leukocyte elastase inhibitors, such as POL-6014; epoxide hydrolase inhibitors, such as GSK-2256294; HNE inhibitors, such as CHF-6333; VIP agonists, such as aviptadil; phosphoinositide-3 kinase delta/gamma inhibitors, such as RV-1729; complement C3 inhibitors, such as APL-1; and G-protein coupled receptor-44 antagonists, such as AM-211.

Other non-limiting examples of active therapeutic agents also include budesonide, adipocell, nitric oxide, PUR-1800, YLP-001, LT-4001, azithromycin, gamunex, QBKPN, sodium pyruvate, MUL-1867, mannitol, MV-130, MEDI-3506, BI-443651, VR-096, OPK-0018, TEV-48107, doxofylline, TEV-46017, OligoG-COPD-5/20, Stempeucel®, ZP-051, lysine acetylsalicylate.

In some embodiments, the other active therapeutic agent may be a vaccine that is active against COPD, including but not limited to MV-130 and GSK-2838497A.

### Combination Therapy for the Treatment of Dengue

The compounds and compositions provided herein are also used in combination with other active therapeutic agents. For the treatment of Flaviviridae virus infections, preferably, the other active therapeutic agent is active against Flaviviridae virus infections, particularly dengue infections. Non-limiting examples of these other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent may be a vaccine for the treatment or prevention of dengue, including but not limited to TetraVax-DV, Dengvaxia®, DPIV-001, TAK-003, live attenuated dengue vaccine, tetravalent dengue fever vaccine, tetravalent DNA vaccine, rDEN2delta30-7169; and DENV-1 PIV.

### Combination Therapy for the Treatment of Ebola

The compounds and compositions provided herein are also used in combination with other active therapeutic agents. For the treatment of Filoviridae virus infections, preferably, the other active therapeutic agent is active against Filoviridae virus infections, particularly Marburg virus, Ebola virus and Cueva virus infections. Non-limiting examples of these other active therapeutic agents are: ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), TKM-Ebola, T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as REGN-3470-3471-3479 and ZMapp.

Other non-limiting active therapeutic agents active against Ebola include an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPC1 inhibitors).

In some embodiments, the other active therapeutic agent may be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The compounds and compositions provided herein may also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The compounds and compositions provided herein are also intended for use with general care provided to patients with Filoviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

### VII. Methods of Treating Viral Infections

The present disclosure provides methods for treating a variety of diseases, such as respiratory syncytial virus (RSV), ebola, Zika, West Nile, Dengue, and HCV using compounds of Formula (Ia), (Ib), (Ic), (Id), (Ie), (If), (Ig), (Ih) or (Ii).

### Paramyxoviridae

In some embodiments, the present disclosure provides methods for treating a Paramyxoviridae infection, comprising administering to an individual (e.g. a human) infected with Paramyxoviridae virus a therapeutically effective amount a compound of the present disclosure or a pharmaceutically acceptable salt thereof. Paramyxoviridae viruses include, but are not limited to, respiratory syncytial virus (RSV).

### Pnuemoviridae

In some embodiments, the present disclosure provides a method of treating a Pneumoviridae virus infection in a human in need thereof, the method comprising administering to the human a therapeutically effective amount of a compound of the present disclosure, or a pharmaceutically acceptable salt thereof. Pneumoviridae viruses include, but are not limited to, respiratory syncytial virus, and human metapneumovirus. In some embodiments, the Pneumoviridae virus infection is a respiratory syncytial virus infection. In some embodiments, the Pneumoviridae virus infection is human metapneumovirus infection.

In some embodiments, the present disclosure provides a method for manufacturing a medicament for treating a Pneumoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Pneumoviridae virus infection. In some embodiments, the Pneumoviridae virus infection is a respiratory syncytial virus infection. In some embodiments, the Pneumoviridae virus infection is human metapneumovirus infection.

In some embodiments, the present disclosure provides a compound of the present disclosure, or a pharmaceutically acceptable salt thereof, for use in the treatment of a Pneumoviridae virus infection in a human in need thereof. In some embodiments, the Pneumoviridae virus infection is a respiratory syncytial virus infection. In some embodiments, the Pneumoviridae virus infection is human metapneumovirus infection.

In certain embodiments, the present disclosure provides methods for treating a RSV infection, comprising administering to an individual (e.g. a human) infected with respiratory syncytial virus a therapeutically effective amount a compound of the present disclosure or a pharmaceutically acceptable salt thereof. Typically, the individual is suffering from a chronic respiratory syncytial viral infection, although it is within the scope of the present disclosure to treat people who are acutely infected with RSV.

In certain embodiments, a method of inhibiting RSV replication is provided, comprising administering a compound of the present disclosure, or a pharmaceutically acceptable salt thereof, to an individual (e.g. a human).

In certain embodiments, the present disclosure provides a method for reducing the viral load associated with RSV infection, wherein the method comprises administering to an individual (e.g. a human) infected with RSV a therapeutically effective amount of a compound of the present disclosure, or a pharmaceutically acceptable salt thereof, wherein the therapeutically effective amount is sufficient to reduce the RSV viral load in the individual.

As described more fully herein, compounds of the present disclosure can be administered with one or more additional therapeutic agent(s) to an individual (e.g. a human) infected with RSV. The additional therapeutic agent(s) can be administered to the infected individual (e.g. a human) at the same time as a compound of the present disclosure or before or after administration of a compound of the present disclosure.

In certain embodiments, a compound of the present disclosure, or a pharmaceutically acceptable salt thereof, for use in treating or preventing a RSV infection is provided. In certain embodiments, a compound of the present disclosure (e.g. a compound of Formula (I)), or a pharmaceutically acceptable salt thereof, for the manufacture of a medicament for treating or preventing a RSV infection is provided.

As described more fully herein, compounds of the present disclosure can be administered with one or more additional therapeutic agent(s) to an individual (e.g. a human) infected with RSV. Further, in certain embodiments, when used to treat or prevent RSV, a compound of the present disclosure may be administered with one or more (e.g. one, two, three, four or more) additional therapeutic agent(s) selected from the group consisting of RSV combination drugs, RSV vaccines, RSV DNA polymerase inhibitors, immunomodulators toll-like receptor (TLR) modulators, interferon alpha receptor ligands, hyaluronidase inhibitors, respiratory syncytial surface antigen inhibitors, cytotoxic T-lymphocyte-associated protein 4 (ipi4) inhibitors, cyclophilin inhibitors, RSV viral entry inhibitors, antisense oligonucleotide targeting viral mRNA, short interfering RNAs (siRNA) and ddRNAi endonuclease modulators, ribonucleotide reductase inhibitors, RSV E antigen inhibitors, covalently closed circular DNA (cccDNA) inhibitors, farnesoid X receptor agonists, RSV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, RSV replication inhibitors, arginase inhibitors, and other RSV drugs.

### Picornaviridae

In some embodiments, the present disclosure provides a method of treating a Picornaviridae virus infection in a human in need thereof, the method comprising administering to the human a therapeutically effective amount of a compound of the present disclosure, or a pharmaceutically acceptable salt thereof. Picornaviridae viruses are eneteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection.

In some embodiments, the present disclosure provides a method for manufacturing a medicament for treating a Picornaviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Picornaviridae virus infection. In some embodiments, the Picornaviridae virus infection is human rhinovirus infection.

In some embodiments, the present disclosure provides a compound of the present disclosure, or a pharmaceutically acceptable salt thereof, for use in the treatment of a Picornaviridae virus infection in a human in need thereof. In some embodiments, the Picornaviridae virus infection is human rhinovirus infection.

### Flaviviridae

In some embodiments, the present disclosure provides a method of treating a Flaviviridae virus infection in a human in need thereof, the method comprising administering to the human a therapeutically effective amount of a compound of the present disclosure, or a pharmaceutically acceptable salt thereof. Representative Flaviviridae viruses include, but are not limited to, dengue, Yellow fever, West Nile, Zika, Japanese encephalitis virus, and Hepatitis C (HCV). In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Japanese ensephalitis virus infection. In some embodiments, the Flaviviridae virus infection is a Hepatitis C virus infection.

In some embodiments, the present disclosure provides a method for manufacturing a medicament for treating a Flaviviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Flaviviridae virus infection. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

In some embodiments, the present disclosure provides a compound of the present disclosure, or a pharmaceutically acceptable salt thereof, for use in the treatment of a Flaviviridae virus infection in a human in need thereof. In some embodiments, the Flaviviridae virus infection is a dengue virus infection. In some embodiments, the Flaviviridae virus infection is a Yellow fever virus infection. In some embodiments, the Flaviviridae virus infection is a West Nile virus infection. In some embodiments, the Flaviviridae virus infection is a Zika virus infection. In some embodiments, the Flaviviridae virus infection is a Hepatitis C virus infection.

### Filoviridae

In some embodiments, the present disclosure provides a method of treating a Filoviridae virus infection in a human in need thereof, the method comprising administering to the human a therapeutically effective amount of a compound of the present disclosure, or a pharmaceutically acceptable salt thereof. Representative Filoviridae viruses include, but are not limited to, ebola and Marburg. In some embodiments, the Filoviridae virus infection is an ebola virus infection.

In some embodiments, the present disclosure provides a method for manufacturing a medicament for treating a Filoviridae virus infection in a human in need thereof, characterized in that the compound of the present disclosure, or a pharmaceutically acceptable salt thereof, is used. In some embodiments, the present disclosure provides use of a compound of the present disclosure, or a pharmaceutically acceptable salt thereof, for the manufacture of a medicament for the treatment in a human of a Filoviridae virus infection. In some embodiments, the Filoviridae virus infection is an ebola virus infection.

In some embodiments, the present disclosure provides a compound of the present disclosure, or a pharmaceutically acceptable salt thereof, for use in the treatment of a Filoviridae virus infection in a human in need thereof. In some embodiments, the Filoviridae virus infection is an ebola virus infection.

### VIII. Methods of Treatment or Prophylaxis of an Exacerbation of a Respiratory Condition by a Virus Infection

The compounds of Formula (Ia), (Ib), (Ic), (Id), (Ie), (If), (Ig), (Ih) or (Ii) can also be used for the treatment or prophylaxis of an exacerbation of a respiratory condition by a viral infection in a human in need thereof.

In some embodiments, the present disclosure provides a method for the treatment or prophylaxis of an exacerbation of a respiratory condition by a viral infection in a human in need thereof, the method comprising administering to the human a therapeutically effective amount of a compound of the present disclosure, or a pharmaceutically acceptable salt thereof, wherein the respiratory condition is chronic obstructive pulmonary disease. In some embodiments, the viral infection is caused by respiratory syncytial virus, rhinovirus or metapneumovirus.

In some embodiments, the present disclosure provides a method for the treatment or prophylaxis of an exacerbation of a respiratory condition by a viral infection in a human in need thereof, the method comprising administering to the human a therapeutically effective amount of the compound of the present disclosure, or a pharmaceutically acceptable salt thereof, wherein the respiratory condition is asthma. In some embodiments, the viral infection is caused by respiratory syncytial virus, rhinovirus, enteroviruses or metapneumovirus.

In some embodiments, the present disclosure provides a method for manufacturing a medicament for the treatment or prophylaxis of an exacerbation of a respiratory condition by a viral infection in a human in need thereof, characterized in that a compound of the present disclosure, or a pharmaceutically acceptable salt thereof, is used, wherein the respiratory condition is chronic obstructive pulmonary disease. In some embodiments, the viral infection is caused by respiratory syncytial virus, rhinovirus or metapneumovirus.

In some embodiments, the present disclosure provides a method for manufacturing a medicament for the treatment or prophylaxis of an exacerbation of a respiratory condition by a viral infection in a human in need thereof, characterized in that the compound of the present disclosure, or a pharmaceutically acceptable salt thereof, is used, wherein the respiratory condition is asthma. In some embodiments, the viral infection is caused by respiratory syncytial virus, rhinovirus, enteroviruses or metapneumovirus.

In some embodiments, the present disclosure provides use of a compound of the present disclosure, or a pharmaceutically acceptable salt thereof, for the manufacture of a medicament for the treatment or prophylaxis in a human of an exacerbation of a respiratory condition by a viral infection, wherein the respiratory condition is chronic obstructive pulmonary disease. In some embodiments, the viral infection is caused by respiratory syncytial virus, rhinovirus or metapneumovirus.

In some embodiments, the present disclosure provides use of the compound of the present disclosure, or a pharmaceutically acceptable salt thereof, for the manufacture of a medicament for the treatment or prophylaxis in a human of an exacerbation of a respiratory condition by a viral infection, wherein the respiratory condition is asthma. In some embodiments, the viral infection is caused by respiratory syncytial virus, rhinovirus, enteroviruses or metapneumovirus.

In some embodiments, the present disclosure provides the compound of the present disclosure, or a pharmaceutically acceptable salt thereof, for use in the treatment or prophylaxis of an exacerbation of a respiratory condition by a viral infection in a human in need thereof, wherein the respiratory condition is chronic obstructive pulmonary disease. In some embodiments, the viral infection is caused by respiratory syncytial virus, rhinovirus or metapneumovirus.

In some embodiments, the present disclosure provides the compound of the present disclosure, or a pharmaceutically acceptable salt thereof, for use in the treatment or prophylaxis of an exacerbation of a respiratory condition by a viral infection in a human in need thereof, wherein the respiratory condition is asthma. In some embodiments, the viral infection is caused by respiratory syncytial virus, rhinovirus, enteroviruses or metapneumovirus.

### IX. Examples

Abbreviations. Certain abbreviations and acronyms are used in describing the experimental details. Although most of these would be understood by one skilled in the art, Table 2 contains a list of many of these abbreviations and acronyms.

TABLE 2
List of abbreviations and acronyms.

| Abbreviation | Meaning |
|---|---|
| Ac | acetate |
| ACN | acetonitrile |
| AIBN | azobisisobutyronitrile |
| Bn | benzyl |
| Bu | butyl |
| Bz | benzoyl |
| BzCl | benzoyl chloride |
| CDI | 1,1′-carbonyldiimidazole |
| DAST | diethylaminosulfur trifluoride |
| DCE | 1,2-dichloroethane |
| DCM | dichloromethane |
| DIPEA | N,N-diisopropylethylamine |
| DMAP | 4-dimethylamiopyridine |
| DMDO | dimethydioxirane |
| DMSO | dimethylsulfoxide |
| DMF | dimethylformamide |
| DMTrCl | 4,4′-dimethoxytritylchloride |
| DMTr | 4,4′-dimethoxytrityl |
| EDCI | N-(3-dimethylaminopropyl)-N′-ethylcarbodiimide |
| | hydrochloride |
| Et | ethyl |
| Imid | imidazole |
| KOtBu | potassium tert-butoxide |
| LC | liquid chromatography |
| MCPBA | meta-chloroperbenzoic acid |
| Me | methyl |
| m/z | mass to charge ratio |
| MS or ms | mass spectrum |
| NIS | N-iodosuccinimide |
| NMP | N-methyl-2-pyrrolidone |
| Ph | phenyl |
| Ph₃P | triphenylphosphine |
| PMB | para-methoxybenzyl |
| PMBCl | para-methoxybenzyl chloride |
| PhOC(S)Cl | phenylchlorothionoformate |
| (PhO)₃PMeI | methyltriphenoxyphosphonium iodide |
| Pyr | pyridine |
| RT | room temperature |
| SFC | supercritical fluid chromatography |
| TBAF | tetrabutylammonium fluoride |
| TBS | tert-butyldimethylsilyl |
| TBSCl | tert-Butyldimethylsilyl chloride |
| TMSN₃ | trimethylsilyl azide |
| TEA | triethylamine |
| TES | triethylsilane |
| TFA | trifluoroacetic acid |
| THF | tetrahydrofuran |
| TMS | trimethylsilyl |
| TMSCl | trimethylsilyl chloride |
| Ts | 4-toluenesulfonyl |
| TsOH | tosylic acid |
| δ | parts per million referenced to residual non- |
| | deuterated solvent peak |

Compounds can be subjected to preparatory HPLC (Phenomenex Gemini 10 u C18 110 Å AXIA 250×21.2 mm column, 30-70% acetonitrile/water gradient with 0.1% TFA). Some compounds are afforded as the TFA salt following this preparatory HPLC process.

### A. Intermediates

### Intermediate 1. (2R,3S,4R,5S)-5-(4-aminopyrrolo[2,1-f][1,2,4]triazin-7-yl)-3,4-dihydroxy-2-(hydroxymethyl)tetrahydrofuran-2-carbonitrile

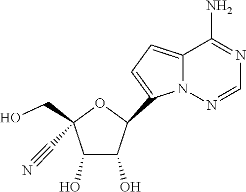 [SMILES: N#C[C@]1(CO)O[C@@H](c2ccc3c(N)ncnn23)[C@H](O)[C@@H]1O]

The product can be prepared according WO2015/069939. For example, pages 43-54 of WO2015/069939 provide a process for preparing the compound, identified as compound 1 in WO2015/069939.

### Intermediate 2. tert-butyl (7-((3aS,4S,6R,6aS)-6-cyano-6-(hydroxymethyl)-2,2-dimethyltetrahydrofuro[3,4-d][1,3]dioxol-4-yl)pyrrolo[2,1-f][1,2,4]triazin-4-yl)carbamate

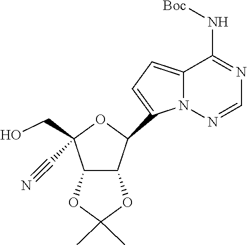 [SMILES: CC(C)(C)OC(=O)Nc1ncnn2c([C@@H]3O[C@](C#N)(CO)[C@H]4OC(C)(C)O[C@@H]34)ccc12]

Compound 14j from WO2015/069939 (21.79 g, 39.93 mmol) in THF (400 mL) was cooled in an ice bath. TBAF 1.0 M in THF (50.0 mL, 50.0 mmol) was added in one portion. The mixture was allowed to come to ambient temperature and stirred for about 30 min. The reaction was determined to be complete by LCMS. The reaction mixture was quenched with water and the organics were removed under reduced pressure. The crude was partitioned between EtOAc and Water. The layers were separated and the aqueous was washed with EtOAc. The organics were combined and dried over sodium sulfate. The solids were filtered off and the solvent removed under reduced pressure. The crude was purified by silica gel chromatography 330 g column 30-100% EtOAc in Hexanes to afford the product. MS m/z=431.74 [M+1]. ¹H NMR (400 MHz, DMSO-d6) δ 10.53 (s, 1H), 8.25 (s, 1H), 7.21 (s, 1H), 7.03 (d, J=4.6 Hz, 1H), 5.77 (t, J=6.1 Hz, 1H), 5.59 (d, J=4.0 Hz, 1H), 5.27 (dd, J=6.7, 4.1 Hz, 1H), 4.94 (d, J=6.7 Hz, 1H), 3.66 (dd, J=6.1, 2.4 Hz, 2H), 1.62 (s, 3H), 1.50 (s, 9H), 1.33 (s, 3H).

### Intermediate 3. (3aS,4R,6S,6aS)-6-(4-aminopyrrolo[2,1-f][1,2,4]triazin-7-yl)-4-(((tert-butyldimethylsilyl)oxy)methyl)-2,2-dimethyltetrahydrofuro[3,4-d][1,3]dioxole-4-carbonitrile

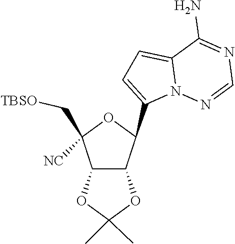 [SMILES: CC1(C)O[C@H]2[C@H](c3ccc4c(N)ncnn34)O[C@](C#N)(CO[Si](C)(C)C(C)(C)C)[C@H]2O1]

The product can be prepared according to WO2015/069939. For example, pages 127-138 of WO2015/069939 provide a process for preparing the compound, identified as compound 14k in WO2015/069939.

### Intermediate 4. (3aS,4R,6S,6aS)-6-(4-aminopyrrolo[2,1-f][1,2,4]triazin-7-yl)-4-(hydroxymethyl)-2,2-dimethyltetrahydrofuro[3,4-d][1,3]dioxole-4-carbonitrile

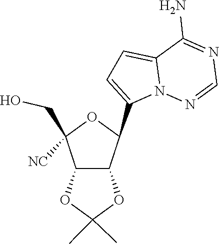 [SMILES: CC1(C)O[C@H]2[C@H](c3ccc4c(N)ncnn34)O[C@](C#N)(CO)[C@H]2O1]

Took up Intermediate 3 (8.41 g, 18.87 mmol) in THF (100 mL). Added TBAF 1.0 M in THF (28.31 mL, 28.31 mmol) in one portion at ambient temperature. Allowed to stir at ambient temperature for 10 min. The reaction was determined to be complete by LCMS. The reaction mixture was quenched with water and the organics were removed under reduced pressure. The crude was partitioned between EtOAc and Water. The layers were separated and the aqueous was washed with EtOAc. The organics were combined and dried over sodium sulfate. The solids were filtered off and the solvent removed under reduced pressure. The crude was purified by silica gel chromatography 120 g column 0-10% CH₃OH in CH₂Cl₂ to afford the product. LC/MS: t_{R}=0.76 min, MS m/z=332.14 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.87-7.80 (m, 3H), 6.85 (d, J=4.5 Hz, 1H), 6.82 (d, J=4.5 Hz, 1H), 5.74 (t, J=5.8 Hz, 1H), 5.52 (d, J=4.2 Hz, 1H), 5.24 (dd, J=6.8, 4.2 Hz, 1H), 4.92 (d, J=6.8 Hz, 1H), 3.65 (dd, J=6.1, 1.7 Hz, 2H), 1.61 (s, 3H), 1.33 (s, 3H).

### Intermediate 5. (2R,3S,4R,5S)-5-(4-aminopyrrolo[2,1-f][1,2,4]triazin-7-yl)-2-(((tert-butyldimethylsilyl)oxy)methyl)-3,4-dihydroxytetrahydrofuran-2-carbonitrile

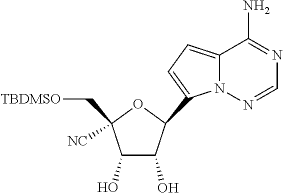 [SMILES: CC(C)(C)[Si](C)(C)OC[C@@]1(C#N)O[C@@H](c2ccc3c(N)ncnn23)[C@H](O)[C@@H]1O]

Dissolved Intermediate 1 (2 g, 6.18 mmol) in 50 mL DMF, to the solution were added tert-butylchlorodimethylsilane (1 g, 7 mmol) and imidazole (1.26 g, 19 mmol). The resulting mixture was stirred at RT for 2 h and the reaction was diluted with EtOAc, washed with NH₄Cl solution, the organic solvent was evaporated and the residue was purified by silica gel column chromatography eluting with 0-100% EtOAc in hexane to afford the product. LCMS: MS m/z=406.36 [M+1], t_{R}=1.45 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.25 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 6. (2R,3S,4S,5S)-5-(4-aminopyrrolo[2,1-f][1,2,4]triazin-7-yl)-2-(((tert-butyldimethylsilyl)oxy)methyl)-2-cyanotetrahydrofuran-3,4-diyl bis(2-methylpropanoate)

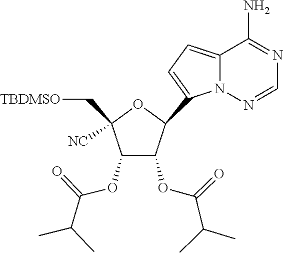 [SMILES: CC(C)C(=O)O[C@H]1[C@H](c2ccc3c(N)ncnn23)O[C@](C#N)(CO[Si](C)(C)C(C)(C)C)[C@H]1OC(=O)C(C)C]

Dissolved Intermediate 5 (1.8 g, 4.44 mmol) in 15 mL THF, to the solution were added isobutyric anhydride (1.54 g, 9.8 mmol) and DMAP (179 mg, 1.45 mmol). The resulting mixture was stirred at RT for 5 min and the reaction was quenched with MeOH and then diluted with EtOAc, washed with brine, the organic solvent was dried over Na₂SO₄ and evaporated under vacuum. The residue was purified by silica gel column chromatography eluting with 0-100% EtOAc in hexane to afford the product. LCMS: MS m/z=546.16 [M+1], t_{R}=1.92 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.88 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 7. (2R,3S,4S,5S)-5-(4-aminopyrrolo[2,1-f][1,2,4]triazin-7-yl)-2-cyano-2-(hydroxymethyl)tetrahydrofuran-3,4-diyl bis(2-methylpropanoate)

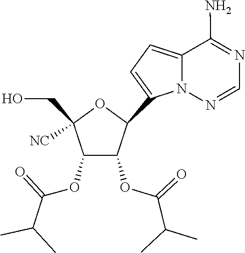 [SMILES: CC(C)C(=O)O[C@H]1[C@H](c2ccc3c(N)ncnn23)O[C@](C#N)(CO)[C@H]1OC(=O)C(C)C]

Dissolved Intermediate 6 (3.2 g, 5.86 mmol) in 25 mL THF in a 100 mL plastic bottle, to the solution was added HF-pyridine (10 g, 0.35 mmol). The resulting mixture was stirred at RT for 3 h and the reaction was quenched with NaHCO₃ and then diluted with EtOAc, washed with brine, the organic solvent was dried over Na₂SO₄ and evaporated under vacuum. The residue was purified by silica gel column chromatography eluting with 0-100% EtOAc in hexane to afford the product. ¹H NMR (400 MHz, Acetonitrile-d3) δ 7.90 (s, 1H), 6.83-6.74 (m, 2H), 6.33 (s, 2H), 5.84-5.74 (m, 2H), 5.62 (d, J=5.4 Hz, 1H), 4.31 (dd, J=8.4, 5.2 Hz, 1H), 3.94 (dd, J=12.2, 5.0 Hz, 1H), 3.87 (dd, J=12.2, 8.4 Hz, 1H), 2.70 (hept, J=7.0 Hz, 1H), 2.56 (hept, J=7.0 Hz, 1H), 1.28-1.17 (m, 6H), 1.12 (dd, J=15.1, 7.0 Hz, 6H). LCMS: MS m/z=432.24 [M+1], t_{R}=1.47 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.74 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 8. cyclopentyl L-alaninate HC salt

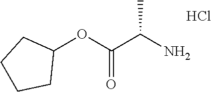

To a mixture of (tert-butoxycarbonyl)-L-alanine (3.95 g, 20.9 mmol), cyclopentanol (1.5 g, 17.4 mmol) and 1-Ethyl-3-(3-dimethylaminopropyl)carbodiimide HCl salt (EDCI) (3.5 g, 22.6 mmol) in acetonitrile (100 mL) was added 4-(Dimethylamino)pyridine (DMAP, 3.2 g, 26.1 mmol). Then the mixture was stirred at room temperature for 2 h, and then the reaction mixture was diluted with EtOAc, washed with brine, dried organic solvent over sodium sulfate, and then concentrated in vacuum. The obtained residue was purified by silica gel chromatography eluting with 0-100% ethyl acetate in hexanes to afford intermediate which was dissolved in 10 mL DCM, to the solution was added 4 N HCl in dioxane (3 mL). The reaction mixture was stirred at RT for 30 min, the solvent was then evaporated and the residue was dried over high vacuum to afford crude product. ¹H NMR (400 MHz, Chloroform-d) δ 8.75-8.42 (m, 2H), 5.20 (tt, J=5.6, 2.5 Hz, 1H), 4.22-4.07 (m, 1H), 1.87-1.58 (m, 8H), 1.54 (dd, J=12.6, 7.2 Hz, 3H).

### Intermediate 9. cyclopropyl L-alaninate HC salt

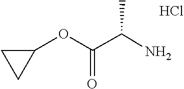

To a mixture of (tert-butoxycarbonyl)-L-alanine (5.86 g, 31 mmol), cyclopropanol (1.5 g, 25.8 mmol) and 1-Ethyl-3-(3-dimethylaminopropyl)carbodiimide HCl salt (EDCI) (5.2 g, 33.6 mmol) in acetonitrile (100 mL) was added 4-(Dimethylamino)pyridine (DMAP, 4.7 g, 38.7 mmol). Then the mixture was stirred at room temperature for 2 h, and then the reaction mixture was diluted with EtOAc, washed with brine, dried organic solvent over sodium sulfate, and then concentrated in vacuum. The obtained residue was purified by silica gel chromatography eluting with 0-100% ethyl acetate in hexanes to afford intermediate which was dissolved in 10 mL DCM, to the solution was added 4 N HCl in dioxane (3 mL). The reaction mixture was stirred at RT for 30 min, the solvent was then evaporated and the residue was dried over high vacuum to afford crude product. ¹H NMR (400 MHz, Chloroform-d) δ 8.68 (s, 2H), 4.22 (tt, J=6.3, 3.2 Hz, 1H), 1.68 (d, J=7.3 Hz, 3H), 1.42 (s, 1H), 0.86-0.69 (m, 2H), 0.70 (dd, J=7.1, 3.6 Hz, 2H).

### Intermediate 10. formacetal 1 and 2: 1,1-Dimethoxy-N,N-dimethylmethanamine and 1-(dimethoxymethyl)-4-methylpiperazine

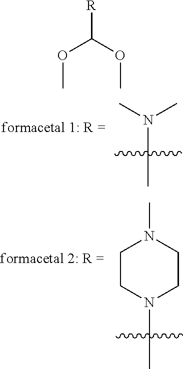

A mixture of N-methylpiperazine (1.5 mL, 15.93 mmol) and DMF-dimethylacetal (1 mL, 7.50 mmol) was heated in a sealed tube at 100° C. for 3 days, concentrated under high vacuum at 60° C. to remove excess N-methyl piperazine, and then used in next reaction. Based on the next reaction's product compositions, the product was a mixture of formacetal 1 and formacetal 2 with ca 1:2 ratio.

### Intermediate 11. (S)-cyclohexyl 2-aminopropanoate hydrochloride

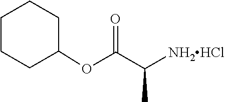

To a mixture of L-alanine (5 g, 56.12 mmol) and cyclohexanol (56 g, 561 mmol) was added TMSCl (20 mL). The resulting mixture was stirred at about 70° C. for about 15 h and concentrated in vacuo at about 80° C., co-evaporated with toluene, dissolved in hexanes, and stirred at about room temperature, during which solid was precipitated. The solid was collected by filtration and the filter cake was washed with 5% EtOAc in hexanes several times, and dried under high vacuum for about 15 h to give the product. ¹H NMR (400 MHz, Chloroform-d) δ 8.76 (s, 3H), 4.85 (tt, J=8.7, 3.8 Hz, 1H), 4.17 (p, J=6.5 Hz, 1H), 1.84 (dd, J=9.9, 5.5 Hz, 2H), 1.70 (d, J=7.3 Hz, 5H), 1.57-1.42 (m, 3H), 1.32 (ddddd, J=20.3, 12.8, 9.9, 6.4, 3.1 Hz, 3H).

### Intermediate 12. (S)-2-ethylbutyl 2-((tert-butoxycarbonyl)amino)-4-methylpentanoate

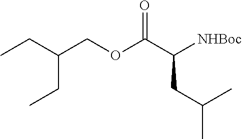 [SMILES: CCC(CC)COC(=O)[C@H](CC(C)C)NC(=O)OC(C)(C)C]

Took up (S)-2-((tert-butoxycarbonyl)amino)-4-methylpentanoic acid (1.09 g, 4.71 mmol) in acetonitrile (10 mL) and added 2-ethyl-1-butanol (2.88 mL, 23.56 mmol) followed by EDCI (878 mg, 5.66 mmol) and DMAP (863 mg, 7.07 mmol) in one portion. Allowed to stir at room temperature overnight. Concentrated and diluted with CH₂Cl₂. Purified by silica gel chromatography 0-40% EtOAc/Hex to afford the product. ¹H NMR (400 MHz, DMSO-d6) δ 7.19 (d, J=8.7 Hz, 1H), 4.00-3.84 (m, 3H), 1.67-1.22 (m, 17H), 0.91-0.80 (m, 12H).

### Intermediate 13. (S)-2-ethylbutyl 2-amino-4-methylpentanoate hydrochloride

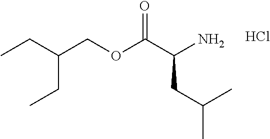

Took up (S)-2-ethylbutyl 2-((tert-butoxycarbonyl)amino)-4-methylpentanoate in CH₂Cl₂ (10 mL) and 4 N HCl in dioxane (10 mL, 40 mmol). Stirred at ambient temperature for 1 h. Concentrated under reduced pressure and co-evaporated with hexanes. Placed under high vacuum for 1 h and the product was used as is without purification for the next step. ¹H NMR (400 MHz, DMSO-d₆) δ 8.43 (s, 3H), 4.08 (d, J=5.6 Hz, 2H), 3.92 (m, 1H), 1.69 (m, 1H), 1.61 (m, 2H), 1.47 (m, 1H), 1.34 (m, 4H), 0.83 (m, 12H).

### Intermediate 14. 2-(Benzyloxy)-2-methylpropyl ((4-nitrophenoxy)(phenoxy)phosphoryl)-L-alaninate

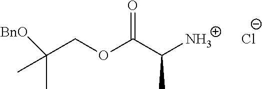 [SMILES: C[C@H]([NH3+])C(=O)OCC(C)(C)OCc1ccccc1 ; [Cl-]]

To a mixture of Boc-L-alanine (1.26 g, 6.66 mmol), 2-benzyloxy-2-methylpropanol (1.0 g, 5.55 mmol), and EDCI (1.12 g, 7.21 mmol) in acetonitrile (20 mL) was added DMAP (2.04 g, 8.32 mmol). Then the mixture was stirred at room temperature for 2 h, then diluted with EtOAc, washed with brine, dried over sodium sulfate, and concentrated in vacuo. The obtained residue was purified by silica gel chromatography (EtOAc 0 to 60% in hexanes) to give a Boc-L-alanine propyl ester, which was dissolved in DCM (10 mL) and 4 N HCl in dioxane (5.5 mL, 22.19 mmol) was added at room temperature. The resulting mixture was stirred at room temperature for 2 h, concentrated in vacuo, re-dissolved in ACN (10 mL), lyophilized overnight to afford the product. ¹H NMR (400 MHz, Chloroform-d) δ 8.82 (s, 3H), 7.42-7.07 (m, 5H), 4.44 (s, 2H), 4.24 (m, 2H), 4.08 (d, J=11.2 Hz, 1H), 1.70 (d, J=7.0 Hz, 3H), 1.28 (d, J=2.4 Hz, 6H). LCMS m/z=251.97 (freebase M+H), t_{R}=0.85 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

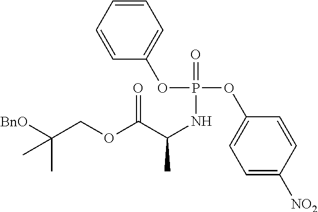 [SMILES: C[C@H](NP(=O)(Oc1ccccc1)Oc1ccc([N+](=O)[O-])cc1)C(=O)OCC(C)(C)OCc1ccccc1]

2-(Benzyloxy)-2-methylpropyl ((4-nitrophenoxy)(phenoxy)phosphoryl)-L-alaninate. To a solution of 2-(Benzyloxy)-2-methylpropyl L-alaninate HCl salt (832 mg, 2.89 mmol) in DCM (20 mL) was added phenyl phosphorodichloridate (0.43 mL, 2.89 mmol) in one portion at −78° C. and triethylamine (0.80 mL, 5.76 mmol) was added dropwise over 5 min at −78° C. The resulting mixture was stirred for 30 min after removal of dry ice bath and cooled to −78° C. and p-nitrophenol (402 mg, 2.89 mmol) was added in one portion and triethylamine (0.40 mL, 2.89 mmol) added over 5 min at −78° C. The resulting mixture was stirred for 50 min after removal of dry ice bath, then diluted with DCM, washed with brine, concentrated in vacuo, and the resulting residue purified by silica gel column chromatography (EtOAc 0 to 60% in hexanes) to give the product. ¹H NMR (400 MHz, Chloroform-d) δ 8.23-8.13 (m, 2H), 7.41-7.27 (m, 3H), 7.28-7.14 (m, 4H), 4.45 (m, 2H), 4.27-4.15 (m, 2H), 4.07 (m, 1H), 3.89 (m, 1H), 1.41 (m, 3H), 1.27 (m, 6H). ³¹P NMR (162 MHz, Chloroform-d) δ −3.10, −3.18. LCMS m/z=528.78 (M+H), t_{R}=1.70 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 15. cyclobutylmethyl ((4-nitrophenoxy)(phenoxy)phosphoryl)-L-alaninate

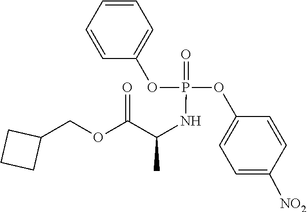 [SMILES: C[C@H](NP(=O)(Oc1ccccc1)Oc1ccc([N+](=O)[O-])cc1)C(=O)OCC1CCC1]

L-Alanine cyclobutylmethyl ester-HCl (1.2 g, 7.16 mmol) was suspended in methylene chloride (10 mL), cooled to −78° C., and phenyl dichlorophosphate (1.07 mL, 7.16 mmol) added quickly. Triethylamine (2.0 mL, 14.32 mmol) was added over 60 min at −78° C. and the resulting mixture was stirred at room temperature for 3 h. The reaction mixture was cooled to 0° C. and 4-nitrophenol (996 mg, 7.16 mmol) was added in one portion. Then triethylamine (1.0 mL, 7.16 mmol) was added over 60 min. Then the mixture was stirred for 3 h at room temperature, filtered, the filtrate concentrated to one third volume, and filtered again. The filtrate was concentrated and the residue purified by silica gel column chromatography (EtOAc 0 to 35% in hexanes) to give the product. ¹H NMR (400 MHz, chloroform-d) δ 8.28-8.16 (m, 2H), 7.45-7.32 (m, 4H), 7.29-7.16 (m, 3H), 4.23-4.01 (m, 3H), 3.95-3.83 (m, 1H), 2.59 (m, 1H), 2.03 (m, 2H), 1.98-1.80 (m, 2H), 1.73 (m, 2H), 1.42 (d, J=3.2 Hz, 1.5H), 1.40 (d, J=3.3 Hz, 1.5H). ³¹P NMR (162 MHz, chloroform-d) δ −3.06, −3.11.

### Intermediate 16. 2-ethylbutyl ((benzyloxy)(4-nitrophenoxy)phosphoryl)-L-alaninate

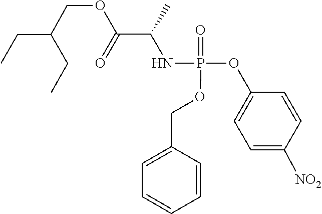 [SMILES: CCC(CC)COC(=O)[C@H](C)NP(=O)(OCc1ccccc1)Oc1ccc([N+](=O)[O-])cc1]

4-Nitrophenyl phosphorodichloridate (2.00 g, 7.81 mmol) and triethylamine (2.18 mL, 15.6 mmol) were sequentially added to a suspension of 2-ethylbutyl L-alaninate hydrochloride (1.091 g, 18.9 mmol) in dichloromethane (23 mL) at 0° C. under an argon atmosphere. After 1 h, benzyl alcohol (0.810 mL, 7.81 mmol) and triethylamine (1.09 mL, 7.81 mmol) were then sequentially added at 0° C., and the resulting mixture was then allowed to warm to rt. After 1 h, the reaction mixture was diluted with dichloromethane (50 mL), washed with saturated an aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, chloroform-d) δ 8.30-8.07 (m, 2H), 7.42-7.28 (m, 7H), 5.18-5.09 (m, 2H), 4.70 (s, 1H), 4.08-3.95 (m, 2H), 3.68 (q, J=9.4 Hz, 1H), 1.55-1.18 (m, 8H), 0.87 (t, J=7.4 Hz, 6H). ³¹P NMR (162 MHz, chloroform-d₁) δ 2.32 (s), 2.28 (s). LCMS: MS m/z=463.00 [M−1], t_{R}=1.56 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

### Intermediate 17. 2-ethylbutyl ((S)-(4-nitrophenoxy)(phenoxy)(phosphoryl)-L-alaninate

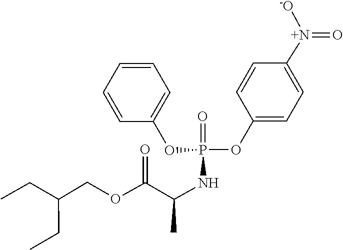

Prepared as described in WO 2016/069825.

### Intermediate 18. isopropyl ((S)-(4-nitrophenoxy)(phenoxy)phosphoryl)-L-alaninate

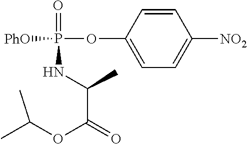 [SMILES: CC(C)OC(=O)[C@H](C)N[P@](=O)(Oc1ccccc1)Oc1ccc([N+](=O)[O-])cc1]

Prepared as described in Cho et al., J. Med. Chem. 2014, 57, 1812-1825.

### Intermediate 19. ethyl ((S)-(4-nitrophenoxy)(phenoxy)phosphoryl)-L-alaninate

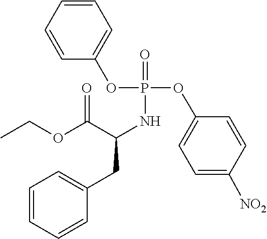 [SMILES: CCOC(=O)[C@H](Cc1ccccc1)NP(=O)(Oc1ccccc1)Oc1ccc([N+](=O)[O-])cc1]

Prepared as described in US20120009147A1.

### Intermediate 20. Cyclopropylmethyl ((4-nitrophenoxy)(phenoxy)phosphoryl)-L-alaninate

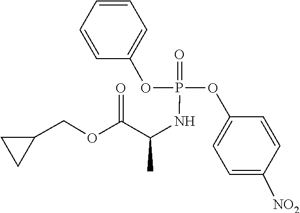 [SMILES: C[C@H](NP(=O)(Oc1ccccc1)Oc1ccc([N+](=O)[O-])cc1)C(=O)OCC1CC1]

L-Alanine cyclopropylmethyl ester-HCl (1.0 g, 5.57 mmol) was suspended in methylene chloride (10 mL), cooled to −78° C., and phenyl dichlorophosphate (0.83 mL, 5.57 mmol) was added quickly. Triethylamine (1.54 mL, 11.13 mmol) in DCM (1.5 mL) was added over 30 min at −78° C. and stirred 30 min. 4-Nitrophenol (774 mg, 5.57 mmol) was added in one portion at −78° C. Then triethylamine (0.77 mL, 7.16 mmol) in DCM (2 mL) was added over 30 min. Then the mixture was stirred for 30 min at the same temperature, washed with water, saturated Na₂CO₃ solution, and brine, and dried with sodium sulfate, and concentrated in vacuo. The residue was purified by silica gel column chromatography (EtOAc 0 to 20% in hexanes) to give the product. ¹H NMR (400 MHz, chloroform-d) δ 8.22 (m, 2H), 7.58-7.29 (m, 4H), 7.32-7.14 (m, 3H), 4.25-4.07 (m, 1H), 4.07-3.80 (m, 3H), 1.44 (d, J=2.9 Hz, 1.5H), 1.42 (d, J=2.9 Hz, 1.5H), 1.26-1.01 (m, 1H), 0.66-0.49 (m, 2H), 0.42-0.15 (m, 2H). ³¹P NMR (162 MHz, chloroform-d) δ −3.07, −3.11. MS m/z=420.97.

### Intermediate 21. 2-(((4-nitrophenoxy)(phenoxy)phosphoryl)amino)ethyl pivalate

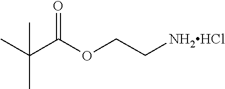

2-aminoethyl pivalate hydrochloride. Pivaloyl chloride (3.82 mL, 31.0 mmol) was added to a solution of tert-butyl (2-hydroxyethyl)carbamate (4.8 mL, 31.0 mmol) and diisopropylethylamine (5.4 mL, 31.0 mmoL) in dichloromethane (150 mL) at RT. After 4 h, the resulting mixture was washed with saturated aqueous sodium bicarbonate solution (150 mL) and brine (150 mL), was dried over anhydrous sodium sulfate, and was concentrated under reduced pressure. The crude colorless oil was taken up into a solution of hydrochloric acid in dioxane (4 M, 50 mL), and was stirred at RT and white solids slowly precipitated from the solution. After 3 h, The solids were collected by vacuum filtration to afford the product. ¹H NMR (400 MHz, CD₃OD) δ 4.32-4.25 (m, 2H), 3.26 (t, J=5.4 Hz, 2H), 1.23 (s, 9H).

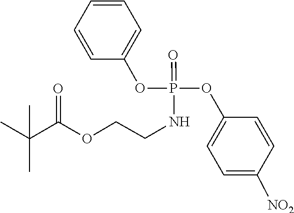 [SMILES: CC(C)(C)C(=O)OCCNP(=O)(Oc1ccccc1)Oc1ccc([N+](=O)[O-])cc1]

2-(((4-nitrophenoxy)(phenoxy)phosphoryl)amino)ethyl pivalate. To a solution of 2-aminoethyl pivalate hydrochloride (0.861 g, 4.74 mmol) and phenyl dichlorophosphate (0.705 mL, 4.74 mmol) in dichloromethane (23 mL) was added triethylamine (1.2 mL, 9.4 mmol) at 0° C. under and argon atmosphere. The resulting mixture was allowed to warm to RT and was stirred for 1.5 h. 4-Nitrophenol (660 mg, 4.74 mmol) and triethylamine (0.66 mL, 4.7 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the product. ¹H NMR (400 MHz, CDCl₃) δ 8.23 (d, J=9.2 Hz, 2H), 7.47-7.31 (m, 4H), 7.29-7.16 (m, 3H), 4.18-4.06 (m, 2H), 3.45-3.31 (m, 2H), 1.17 (s, 9H). ³¹P NMR (162 MHz, DMSO-d₆) δ −1.48 (s). MS m/z=422.95 [M+1].

### Intermediate 22. (2S)-tetrahydro-2H-pyran-4-yl 2-(((4-nitrophenoxy)(phenoxy)phosphoryl)amino)propanoate

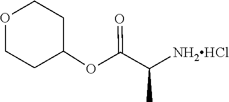

(S)-tetrahydro-2H-pyran-4-yl 2-aminopropanoate hydrochloride. To a mixture of L-alanine (500 mg, 5.61 mmol) and tetrahydro-2H-pyran-4-ol (5 g, 49.0 mmol) was added TMSCl (2 mL). The resulting mixture was stirred at 70° C. for 15 h and concentrated in vacuo and the resulting solid was tritulated with 5% EtOAc in hexanes, filtered, and washed with 5% EtOAc in hexanes several times, and dried under high vacuum for 15 h to give the product which was used in next reaction without any characterization.

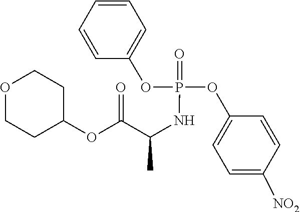 [SMILES: C[C@H](NP(=O)(Oc1ccccc1)Oc1ccc([N+](=O)[O-])cc1)C(=O)OC1CCOCC1]

(2S)-tetrahydro-2H-pyran-4-yl 2-(((4-nitrophenoxy)(phenoxy)phosphoryl)amino)propanoate. (S)-tetrahydro-2H-pyran-4-yl 2-aminopropanoate hydrochloride (1.33 g, 6.34 mmol) was dissolved in methylene chloride (15 mL), cooled to −78° C., and phenyl dichlorophosphate (1.137 mL, 7.61 mmol) added quickly. Triethylamine (2.2 mL, 15.2 mmol) was added over 30 min at −78° C. and the resulting mixture was stirred for 30 min at −78° C. Then 4-nitrophenol (882 mg, 6.34 mmol) was added in one portion and triethylamine (1.1 mL, 7.61 mmol) was added over 30 min at −78° C. The mixture was stirred for 30 min at −78° C., washed with water twice and brine, dried over sodium sulfate, and concentrated in vacuo. The residue was purified by silica gel column chromatography (EtOAc 0 to 70% in hexanes) to give the product. ¹H NMR (400 MHz, Chloroform-d) δ 8.22 (m, 2H), 7.49-7.06 (m, 7H), 4.95 (m, 1H), 4.14 (m, 1H), 4.07-3.80 (m, 3H), 3.52 (m, 2H), 1.95-1.81 (m, 2H), 1.64 m, 2H), 1.42 (m, 3H). ³¹P NMR (162 MHz, Chloroform-d) δ −3.09, −3.13. MS m/z=451 (M+H)⁺.

### Intermediate 23. (S)-1-methylpyrrolidin-3-yl ((4-nitrophenoxy)(phenoxy)phosphoryl)-L-alaninate

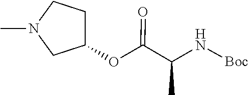 [SMILES: C[C@H](NC(=O)OC(C)(C)C)C(=O)O[C@H]1CCN(C)C1]

(S)-1-methylpyrrolidin-3-yl (tert-butoxycarbonyl)-L-alaninate. Boc-L-Alanine (2.1 g, 11 mmol) and (R)-3-hydroxy-1-methylpyrrolidine (1.1 mL, 10 mmol) were dissolved in anhydrous THF (20 mL). Triphenylphosphine (3.4 g, 13 mmol) was added in one portion. Diisopropyl azodicarboxylate (2.4 mL, 12 mmol) was added dropwise. Reaction was stirred for 2 hrs. More diisopropyl azodicarboxylate (240 uL, 1.2 mmol) was added dropwise, and the reaction was stirred for 16 hrs. Reaction was diluted with EtOAc (10 mL) and washed with saturated aqueous sodium bicarbonate solution (10 mL). Organic was then extracted with 5% aqueous citric acid solution (30 mL). Citric acid extract was washed with EtOAc (2×5 mL). Citric acid portion was basified with 1 N aqueous NaOH solution to give pH of 9 and extracted with EtOAc (2×10 mL). Organic extracts were combined, dried over anhydrous sodium sulfate and then concentrated under reduced pressure to give the product. ¹H NMR (400 MHz, chloroform-d) δ 5.24 (m, 1H), 5.01 (m, 1H), 4.27 (m, 1H), 2.88-2.69 (m, 2H), 2.64 (m, 1H), 2.37 (s, 3H), 2.29 (m, 1H), 1.96-1.80 (m, 1H), 1.44 (s, 9H), 1.37 (d, J=7.2 Hz, 3H).

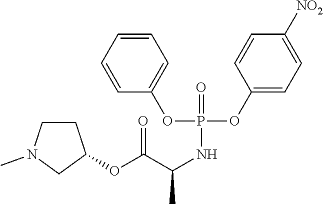 [SMILES: C[C@H](NP(=O)(Oc1ccccc1)Oc1ccc([N+](=O)[O-])cc1)C(=O)O[C@H]1CCN(C)C1]

(S)-1-methylpyrrolidin-3-yl ((4-nitrophenoxy)(phenoxy)phosphoryl)-L-alaninate. (S)-1-methylpyrrolidin-3-yl (tert-butoxycarbonyl)-L-alaninate (545 mg, 2 mmol) was mixed with 10 mL of 4 N HCl in dioxane and stirred for 1 hr. Reaction was concentrated under reduced pressure to give foam which was then mixed with 20 mL anhydrous DCM and stirred under atmospheric nitrogen in an ice bath. Phenyl dichlorophosphate (298 uL, 2 mmol) was added to reaction in one portion. Reaction was stirred for 15 mins. Triethylamine (837 uL, 6 mmol) was added to the reaction dropwise. Reaction was stirred for 1 hr. Triethylamine (279 μL, 2 mmol) was added to the reaction dropwise and then stirred for 30 mins. p-Nitrophenol (250 mg, 1.8 mmol) was added in one portion. Reaction mixture was stirred for 16 hrs. Reaction was diluted with DCM (20 mL) and washed with water (5×20 mL). Organic was dried over anhydrous sodium sulfate and concentrated under reduced pressure. The crude residue was purified via SiO₂ column chromatography (12 g SiO₂ Combiflash HP Gold Column, 0-10% methanol/DCM). Fractions were combined and concentrated under reduced pressure to give the product. ¹H NMR (400 MHz, chloroform-d) δ 8.28-8.15 (m, 2H), 7.46-7.28 (m, 4H), 7.28-7.13 (m, 3H), 5.17 (m, 1H), 4.21-4.04 (m, 1H), 4.01-3.85 (m, 1H), 2.81 (m, 1H), 2.70-2.55 (m, 2H), 2.35 (s, 3H), 2.33-2.21 (m, 2H), 1.84-1.70 (m, 1H), 1.39 (m, 3H). ³¹P NMR (162 MHz, chloroform-d) δ −3.16, −3.21. LCMS: MS m/z=450.3 [M+1]; 448.1 [M−1], t_{R}=1.15 min; LC system: Thermo Dionex Ultimate 3000 UHPLC; Column: Phenomenex Kinetex 2.6p C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.61 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 24. (R)-1-methylpyrrolidin-3-yl ((4-nitrophenoxy)(phenoxy)phosphoryl)-L-alaninate

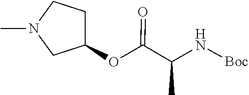 [SMILES: C[C@H](NC(=O)OC(C)(C)C)C(=O)O[C@@H]1CCN(C)C1]

(R)-1-methylpyrrolidin-3-yl (tert-butoxycarbonyl)-L-alaninate. Boc-L-Alanine (5.2 g, 27.5 mmol) and (R)-3-hydroxy-1-methylpyrrolidine (2.74 mL, 25 mmol) were dissolved in anhydrous THF (25 mL). N,N′-Diisopropylcarbodiimide (4.67 mL, 30 mmol) was added dropwise. Reaction was stirred for 2 hrs. More N,N′-diisopropylcarbodiimide (467 uL, 3 mmol) was added dropwise, and the reaction was stirred for 2 hrs. More N,N′-diisopropyl carbodiimide (467 uL, 3 mmol) was added dropwise, and the reaction was stirred for 16 hrs.

Reaction was diluted with EtOAc (25 mL) and stirred for 10 mins. Solid was filtered off and washed with small amount of EtOAc. Filtrate was washed with saturated aqueous sodium bicarbonate solution (3×10 mL). Organic was then extracted with 5% aqueous citric acid solution (50 mL). Citric acid extract was washed with EtOAc (5 mL). Citric acid portion was basified with 1 N aqueous NaOH solution to give pH of 9 and then extracted with EtOAc (3×15 mL). Organic extracts were combined, dried over anhydrous sodium sulfate and then concentrated under reduced pressure to give the product. ¹H NMR (400 MHz, chloroform-d) δ 5.28-5.18 (m, 1H), 5.02 (m, 1H), 4.28 (m, 1H), 2.84-2.75 (m, 1H), 2.69 (d, J=4.2 Hz, 2H), 2.36 (s, 3H), 2.34-2.22 (m, 2H), 1.87-1.76 (m, 1H), 1.44 (s, 9H), 1.37 (d, J=7.2 Hz, 3H).

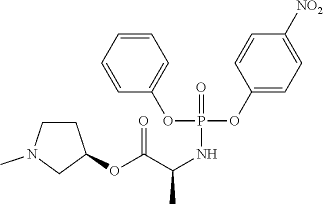 [SMILES: C[C@H](NP(=O)(Oc1ccccc1)Oc1ccc([N+](=O)[O-])cc1)C(=O)O[C@@H]1CCN(C)C1]

(R)-1-methylpyrrolidin-3-yl ((4-nitrophenoxy)(phenoxy)phosphoryl)-L-alaninate. (R)-1-methylpyrrolidin-3-yl (tert-butoxycarbonyl)-L-alaninate (3.9 g, 14.3 mmol) was mixed with 30 mL of 4 N HCl in dioxane and stirred for 3 hrs. Reaction was concentrated under reduced pressure to give foam which was then mixed with 30 mL anhydrous DCM and stirred under atmospheric nitrogen in an ice bath. Phenyl dichlorophosphate (2.34 mL, 15.75 mmol) was added to reaction in one portion. Reaction was stirred for 15 mins. Triethylamine (4.4 mL, 31.5 mmol) was mixed with anhydrous DCM (5 mL) and added to the reaction dropwise. Reaction was stirred for 1 hr. Triethylamine (2.2 mL, 15.75 mmol) was mixed with anhydrous DCM (3 mL) and added to the reaction dropwise. Reaction was stirred for 15 mins. p-Nitrophenol (1.8 g, 12.87 mmol) was added in one portion. Reaction mixture was stirred for 2 hrs.

Reaction was diluted with DCM (20 mL) and washed with aqueous sodium bicarbonate solution (3×20 mL). Organic was dried over anhydrous sodium sulfate and concentrated under reduced pressure. The crude residue was purified via SiO₂ column chromatography (40 g SiO₂ Combiflash HP Gold Column, 0-10% methanol/DCM). Fractions were combined and concentrated under reduced pressure to give the product. ¹H NMR (400 MHz, chloroform-d) δ 8.29-8.15 (m, 2H), 7.48-7.29 (m, 4H), 7.29-7.13 (m, 3H), 5.20 (m, 1H), 4.21-4.07 (m, 1H), 3.99 (m, 1H), 2.86 (m, 1H), 2.70 (m, 1H), 2.63 (m, 1H), 2.37 (m, 3H), 2.35-2.21 (m, 2H), 1.86-1.73 (m, 1H), 1.40 (m, 3H). ³¹P NMR (162 MHz, chloroform-d) δ −3.12, −3.14. LCMS: MS m/z=450.3 [M+1]; 448.1 [M−1], t_{R}=1.24 min; LC system: Thermo Dionex Ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.63 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 25. (2S)-cyclohexyl 2-(((4-nitrophenoxy)(phenoxy)phosphoryl)amino)propanoate

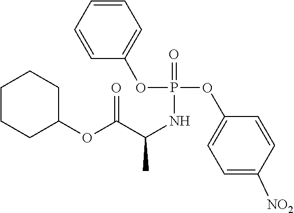 [SMILES: C[C@H](NP(=O)(Oc1ccccc1)Oc1ccc([N+](=O)[O-])cc1)C(=O)OC1CCCCC1]

Intermediate 11 (3.4 g, 16.37 mmol) was dissolved in methylene chloride (45 mL), cooled to −78° C., and phenyl dichlorophosphate (2.45 mL, 16.37 mmol) added quickly. Triethylamine (4.54 mL, 32.74 mmol) was added over 60 min at −78° C. and then 4-nitrophenol (2277 mg, 16.37 mmol) was added in one portion. Triethylamine (2.27 mL, 16.37 mmol) was added over 60 min at −78° C. The resulting mixture was stirred for 2 h at −78° C., diluted with methylene chloride (100 mL), washed with water twice and brine, dried over sodium sulfate, and concentrated in vacuo. The residue was purified by silica gel column chromatography (EtOAc 0 to 20% in hexanes) to give the product. ¹H NMR (400 MHz, Chloroform-d) δ 8.22 (m, 2H), 7.46-7.30 (m, 4H), 7.29-7.09 (m, 3H), 4.76 (m, 1H), 4.20-4.02 (m, 1H), 3.92 (m, 1H), 1.87-1.64 (m, 4H), 1.54 (m, 2H), 1.46-1.18 (m, 7H). ³¹P NMR (162 MHz, Chloroform-d) δ −2.94, −3.00. MS m/z=449 (M+H)⁺.

### Intermediate 26. tert-butyl 4-(((2S)-2-(((4-nitrophenoxy)(phenoxy)phosphoryl)amino)propanoyl)oxy)piperidine-1-carboxylate

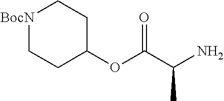 [SMILES: C[C@H](N)C(=O)OC1CCN(C(=O)OC(C)(C)C)CC1]

tert-butyl 4-((L-alanyl)oxy)piperidine-1-carboxylate. To a mixture of ((benzyloxy)carbonyl)-L-alanine (1.26 g, 5.65 mmol), tert-butyl 4-hydroxypiperidine-1-carboxylate (5.68 g, 28.22 mmol), and EDCI (1.05 g, 6.77 mmol) in acetonitrile (15 mL) was added DMAP (1.03 g, 8.47 mmol). Then the mixture was stirred at room temperature for 15 h, diluted with EtOAc, washed with brine, dried over sodium sulfate, and concentrated in vacuo. The obtained residue was purified by silica gel chromatography (EtOAc 0 to 100% in hexanes) to give a Cbz-L-alanine piperidyl ester, which was dissolved in THF (10 mL) and 20% palladium hydroxide (400 mg) on carbon was added. The resulting mixture was stirred under H₂ gas for 2 h, filtered, and the filtrate concentrated in vacuo. The obtained residue was dried under high vacuum to afford the product. ¹H NMR (400 MHz, Chloroform-d) δ 4.95 (tt, J=7.9, 3.8 Hz, 1H), 3.79-3.62 (m, 2H), 3.56 (q, J=7.0 Hz, 1H), 3.25 (ddd, J=13.6, 8.5, 3.7 Hz, 2H), 1.85 (ddd, J=13.4, 6.4, 3.4 Hz, 2H), 1.73 (s, 2H), 1.62 (ddq, J=12.7, 8.7, 4.3, 3.9 Hz, 2H), 1.46 (s, 9H), 1.34 (d, J=7.0 Hz, 3H). MS m/z=273 [M+H].

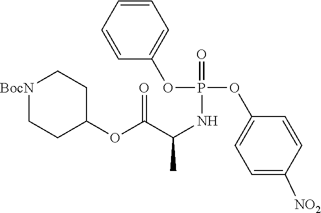 [SMILES: C[C@H](NP(=O)(Oc1ccccc1)Oc1ccc([N+](=O)[O-])cc1)C(=O)OC1CCN(C(=O)OC(C)(C)C)CC1]

tert-butyl 4-(((2S)-2-(((4-nitrophenoxy)(phenoxy)phosphoryl)amino)propanoyl)oxy)piperidine-1-carboxylate. tert-butyl 4-((L-alanyl)oxy)piperidine-1-carboxylate (0.9 g, 3.31 mmol) was dissolved in methylene chloride (10 mL), cooled to −78° C., and phenyl dichlorophosphate (0.49 mL, 3.31 mmol) added quickly. Triethylamine (0.46 mL, 3.31 mmol) was added over 30 min at −78° C. and 4-nitrophenol (460 mg, 3.31 mmol) was added in one portion. Then triethylamine (0.49 mL, 3.31 mmol) was added over 30 min at −78° C. The resulting mixture was stirred for 2 h at −78° C., diluted with methylene chloride, washed with water twice and brine, dried over sodium sulfate, and concentrated in vacuo. The residue was purified by silica gel column chromatography (EtOAc 0 to 70% in hexanes) to afford the product. ¹H NMR (400 MHz, Chloroform-d) δ 8.23 (m, 2H), 7.42-7.31 (m, 4H), 7.25-7.16 (m, 3H), 4.93 (m, 1H), 4.26-4.03 (m, 1H), 3.85 (m, 1H), 3.75-3.56 (m, 2H), 3.21 (m, 2H), 1.91-1.75 (m, 2H), 1.66-1.48 (m, 2H), 1.46 (s, 9H), 1.44-1.38 (m, 3H). ³¹P NMR (162 MHz, Chloroform-d) δ −3.07, −3.13. MS m/z=550 (M+H)⁺.

### Intermediate 27. trans-4-(trifluoromethyl)cyclohexyl ((4-nitrophenoxy)(phenoxy)phosphoryl)-L-alaninate

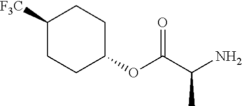 [SMILES: C[C@H](N)C(=O)O[C@H]1CC[C@H](C(F)(F)F)CC1]

trans-4-(trifluoromethyl)cyclohexyl L-alaninate. The product was prepared from Cbz-l-alanine (900 mg, 4.03 mmol) and trans-4-(trifluoromethyl)cyclohexan-1-ol (1.02 g, 6.05 mmol) in a manner similar to that described for Intermediate 26. MS m/z=240 [M+H].

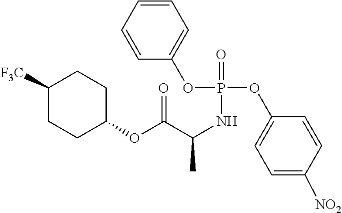 [SMILES: C[C@H](NP(=O)(Oc1ccccc1)Oc1ccc([N+](=O)[O-])cc1)C(=O)O[C@H]1CC[C@H](C(F)(F)F)CC1]

trans-4-(trifluoromethyl)cyclohexyl ((4-nitrophenoxy)(phenoxy)phosphoryl)-L-alaninate. The product (840 mg) was prepared as isomeric mixture from trans-4-(trifluoromethyl)cyclohexyl L-alaninate (974 mg, 4.07 mmol) in a manner similar to that described for Intermediate 25. ¹H NMR (400 MHz, Chloroform-d) δ 8.27-8.19 (m, 2H), 7.43-7.31 (m, 4H), 7.26-7.16 (m, 3H), 4.68 (m, 1H), 4.11 (m, 1H), 3.84 (m, 1H), 2.02 (m, 4H), 1.50-1.27 (m, 8H). ¹⁹F NMR (377 MHz, Chloroform-d) δ −73.91 (d, J=7.7 Hz). ³¹P NMR (162 MHz, Chloroform-d) δ −3.08, −3.12. MS m/z=517 [M+H].

The product was separated by Chiralpak SFC (Chiralpak IF 20×250 mm column, 30% isopropanol) to afford Intermediate 28 and Intermediate 29:

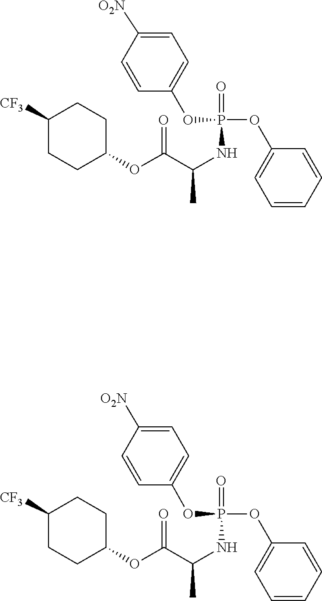 [SMILES: C[C@H](N[P@@](=O)(Oc1ccccc1)Oc1ccc([N+](=O)[O-])cc1)C(=O)O[C@H]1CC[C@H](C(F)(F)F)CC1 ; C[C@H](N[P@](=O)(Oc1ccccc1)Oc1ccc([N+](=O)[O-])cc1)C(=O)O[C@H]1CC[C@H](C(F)(F)F)CC1]

Intermediate 28. trans-4-(trifluoromethyl)cyclohexyl ((R)-(4-nitrophenoxy)(phenoxy)phosphoryl)-L-alaninate. First eluting diastereomer of Intermediate 27: ¹H NMR (400 MHz, Chloroform-d) δ 8.22 (d, J=9.1 Hz, 2H), 7.42-7.31 (m, 4H), 7.29-7.16 (m, 3H), 4.69 (tt, J=10.7, 4.2 Hz, 1H), 4.19-4.04 (m, 1H), 3.90 (dd, J=11.9, 9.5 Hz, 1H), 2.12-1.97 (m, 5H), 1.52-1.21 (m, 7H). ¹⁹F NMR (376 MHz, Chloroform-d) δ −73.90 (d, J=7.7 Hz). ³¹P NMR (162 MHz, Chloroform-d) δ −3.07.

Intermediate 29. trans 4-(trifluoromethyl)cyclohexyl ((S)-(4-nitrophenoxy)(phenoxy)phosphoryl)-L-alaninate. Second eluting diastereomer of Intermediate 27: ¹H NMR (400 MHz, Chloroform-d) δ 8.21 (d, J=9.08 Hz, 2H), 7.42-7.31 (m, 4H), 7.26-7.13 (m, 3H), 4.67 (tt, J=10.8, 4.2 Hz, 1H), 4.11 (ddt, J=15.8, 8.9, 7.1 Hz, 1H), 3.97 (dd, J=12.0, 9.4 Hz, 1H), 2.07-1.91 (m, 5H), 1.51-1.19 (m, 7H). ¹⁹F NMR (376 MHz, Chloroform-d) δ −73.90 (d, J=7.9 Hz). ³¹P NMR (162 MHz, Chloroform-d) δ −3.08.

### Intermediate 30. 1-Methylpiperidin-4-yl((4-nitrophenoxy) (phenoxy)phosphoryl)-L-alaninate

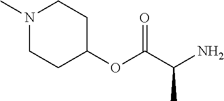

1-Methylpiperidin-4-yl L-alaninate. To a mixture of N-Cbz-L-alanine (1.047 g, 4.688 mmol), 4-hydroxy-N-methylpiperidine (450 mg, 3.907 mmol), and EDCI (788 mg, 5.079 mmol) in acetonitrile (20 mL) was added DMAP (716 mg, 5.861 mmol). Then the mixture was stirred at room temperature for 15 h, then diluted with EtOAc, washed with brine, dried over sodium sulfate, and concentrated in vacuo. The obtained residue was purified by silica gel chromatography (MeOH 0 to 10% in DCM) to give an Cbz-L-alanine 4-piperidyl ester, which was dissolved in THF (10 mL) and 20% Pd(OH)₂ (300 mg, 0.427 mmol) was added at room temperature. The resulting mixture was stirred under H₂ gas at room temperature for 2 h, filtered, concentrated in vacuo, co-evaporated with DCM several times, and dried under high vacuum overnight to afford the product. ¹H NMR (400 MHz, Chloroform-d) δ 4.81 (td, J=8.3, 7.7, 3.8 Hz, 1H), 3.52 (q, J=7.0 Hz, 1H), 2.63 (s, 2H), 2.29 (s, 5H), 2.14-1.86 (m, 4H), 1.73 (ddt, J=12.9, 8.8, 4.5 Hz, 2H), 1.32 (d, J=7.0 Hz, 3H). LCMS: MS m/z=187.09 [M+1]; t_{R}=0.12 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

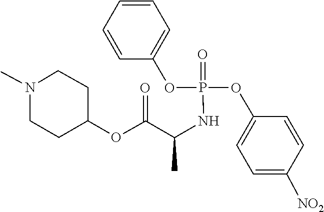 [SMILES: C[C@H](NP(=O)(Oc1ccccc1)Oc1ccc([N+](=O)[O-])cc1)C(=O)OC1CCN(C)CC1]

1-Methylpiperidin-4-yl((4-nitrophenoxy) (phenoxy)phosphoryl)-L-alaninate. To a solution of 1-Methylpiperidin-4-yl L-alaninate (360 mg, 1.706 mmol) in DCM (10 mL) was added phenyl phosphorodichloridate (0.255 mL, 1.706 mmol) in one portion at −78° C. and then triethylamine (0.24 mL, 1.706 mmol) in DCM (2.76 mL) was added over 30 min at −78° C. The resulting mixture was stirred for 30 min after removal of dry ice bath and then recooled to −78° C. p-Nitrophenol (0.237 g, 1.706 mmol) was added in one portion and triethylamine (0.237 mL, 1.706 mmol) added over 30 min at −78° C. The resulting mixture was stirred for 30 min after removal of dry ice bath, then diluted with EtOAc, washed with water and brine, concentrated in vacuo, and the resulting residue purified by silica gel column chromatography (MeOH 0 to 10% in DCM) to afford the product. ¹H NMR (400 MHz, Chloroform-d) δ 8.28-8.15 (m, 2H), 7.36 (m, 4H), 7.25-7.17 (m, 3H), 4.80 (s, 1H), 4.19-4.04 (m, 1H), 3.93 (m, 1H), 2.64 (s, 2H), 2.31 (m, 5H), 1.90 (m, 2H), 1.78-1.67 (m, 2H), 1.47-1.33 (m, 3H). ³¹P NMR (162 MHz, Chloroform-d) δ −3.04, −3.07. LCMS: MS m/z=464.32 [M+1]; t_{R}=0.74 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 31. (tetrahydro-2H-pyran-4-yl)methyl ((4-nitrophenoxy)(phenoxy)phosphoryl)-L-alaninate

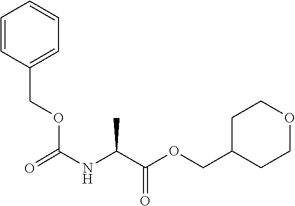 [SMILES: C[C@H](NC(=O)OCc1ccccc1)C(=O)OCC1CCOCC1]

(tetrahydro-2H-pyran-4-yl)methyl ((benzyloxy)carbonyl)-L-alaninate. Cbz-L-Ala (446 mg, 2 mmol) was dissolved in anhydrous MeCN (10 mL). EDCI (422 mg, 2.2 mmol) was added in one portion and the reaction was stirred for 15 mins. Tetrahydropyran-4-methanol (279 uL, 2.4 mmol) was added. DMAP (269 mg, 2.2 mmol) was then added in one portion. Reaction was stirred for 16 hrs.

Reaction was diluted reaction with EtOAc (30 mL) and washed with 5% aqueous citric acid solution (10 mL), followed with saturated aqueous sodium bicarbonate solution (10 mL) and finally with brine (10 mL). Organic was dried over anhydrous sodium sulfate and concentrated under reduced pressure. The crude residue was purified via SiO₂ column chromatography (12 g SiO₂ Combiflash HP Gold Column, 0-80% ethyl acetate/hexanes). Fractions were combined and concentrated under reduced pressure to afford the product. ¹H NMR (400 MHz, Chloroform-d) δ 7.40-7.28 (m, 5H), 5.28 (d, J=7.9 Hz, 1H), 5.11 (s, 2H), 4.39 (t, J=7.4 Hz, 1H), 4.07-3.84 (m, 4H), 3.38 (t, J=11.7 Hz, 2H), 1.92 (s, 1H), 1.68-1.50 (m, 3H), 1.39 (m, 4H).

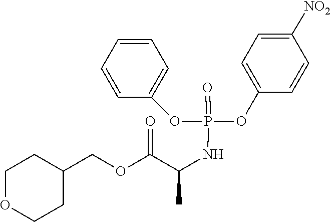 [SMILES: C[C@H](NP(=O)(Oc1ccccc1)Oc1ccc([N+](=O)[O-])cc1)C(=O)OCC1CCOCC1]

(tetrahydro-2H-pyran-4-yl)methyl ((4-nitrophenoxy)(phenoxy)phosphoryl)-L-alaninate. (tetrahydro-2H-pyran-4-yl)methyl ((benzyloxy)carbonyl)-L-alaninate (530 mg, 1.65 mmol) was dissolved in anhydrous THF (12 mL). 10% Pd/C Degussa type was added and the reaction mixture was stirred under atmospheric hydrogen for 2 hrs. Catalyst was filtered and the filtrate was used without purification.

Phenyl dichlorophosphate (294 uL, 1.98 mmol) was dissolved in anhydrous DCM (10 mL) and stirred in an ice bath under atmospheric nitrogen. Above THF solution was added to the reaction dropwise and then stirred for 10 mins. Triethylamine (300 uL, 2.15 mmol) was added dropwise and then stirred for 30 mins. p-Nitrophenol (207 mg, 1.49 mmol) and triethylamine (300 uL, 2.15 mmol) were added. Ice bath was removed and the reaction mixture was stirred for 14 hrs at RT.

Reaction was diluted with EtOAc (30 mL) and washed with 0.2 M sodium carbonate solution (2×10 mL) and followed with brine (10 mL). Organic was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions were combined and concentrated under reduced pressure to afford the product. ¹H NMR (400 MHz, Chloroform-d) δ 8.23 (d, J=9.0 Hz, 2H), 7.45-7.30 (m, 4H), 7.30-7.16 (m, 3H), 4.23-4.07 (m, 2H), 3.97 (m, 4H), 3.85 (t, J=10.5 Hz, 1H), 3.35 (t, J=11.8 Hz, 2H), 1.99-1.79 (m, 1H), 1.56 (d, J=8.4 Hz, 3H), 1.48-1.29 (m, 4H). ³¹P NMR (162 MHz, Chloroform-d) δ −3.13 (s), −3.16 (s). MS m/z=464.9 [M+1]; 463.1 [M−1].

### Intermediate 32. trans-4-(tert-butyl)cyclohexyl ((S)-(4-nitrophenoxy)(phenoxy)phosphoryl)-L-alaninate

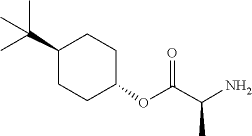 [SMILES: C[C@H](N)C(=O)O[C@H]1CC[C@H](C(C)(C)C)CC1]

trans-4-(tert-butyl)cyclohexyl L-alaninate. The product (845 mg) was prepared from Cbz-l-alanine (960 mg, 4.03 mmol) and trans-4-(tert-butyl)cyclohexanol (1.0 g, 6.45 mmol) in a manner similar to that described for Intermediate 26. ¹H NMR (400 MHz, Chloroform-d) δ 4.65 (tt, J=11.2, 4.5 Hz, 1H), 3.51 (q, J=7.1 Hz, 1H), 2.07-1.93 (m, 2H), 1.87-1.73 (m, 4H), 1.40-1.23 (m, 4H), 1.19-0.94 (m, 4H), 0.85 (d, J=2.6 Hz, 9H). MS m/z=228 [M+H].

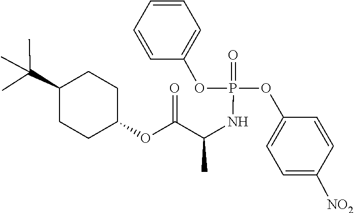 [SMILES: C[C@H](NP(=O)(Oc1ccccc1)Oc1ccc([N+](=O)[O-])cc1)C(=O)O[C@H]1CC[C@H](C(C)(C)C)CC1]

trans-4-(tert-butyl)cyclohexyl ((4-nitrophenoxy)(phenoxy) phosphoryl)-L-alaninate. The product (520 mg) was prepared as isomeric mixture from trans-4-(tert-butyl)cyclohexyl L-alaninate (420 mg, 1.85 mmol) in a manner similar to that described for Intermediate 25. ¹H NMR (400 MHz, Chloroform-d) δ 8.27-8.19 (m, 2H), 7.37 (m, 4H), 7.28-7.16 (m, 3H), 4.62 (m, 1H), 4.17-4.00 (m, 1H), 3.88 (m, 1H), 1.95 (m, 2H), 1.80 (m, 2H), 1.39 (m, 3H), 1.35-1.22 (m, 2H), 1.15-0.92 (m, 3H), 0.85 (s, 9H). ³¹P NMR (162 MHz, Chloroform-d) δ −2.98, −3.04. MS m/z=505 [M+H].

### Intermediate 33. ((1r, 4S)-4-(trifluoromethyl)cyclohexyl)methyl ((4-nitrophenoxy)(phenoxy)phosphoryl)-L-alaninate

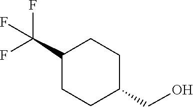

((1s, 4s)-4-(trifluoromethyl)cyclohexyl)methanol. To an ice cold solution of (1s,4s)-4-(trifluoromethyl)cyclohexane carboxylic acid (3 g, 15.29 mmol) in anhydrous tetrahydrofuran (40 mL) was added lithium aluminum hydride (0.871 g, 22.94 mmol) portion wise in 30 min. The reaction mixture was stirred at room temperature for 3 h. Cooled to 0° C. and quenched with water (0.8 mL), 5 N aqueous sodium hydroxide (0.8 mL) followed by water (2.4 mL). Solids separated were filtered and filtrate was diluted with ethyl acetate and saturated aqueous sodium bicarbonate solution. Organic layer was separated, washed with brine and dried over sodium sulfate. Ethyl acetate was filtered and concentrated under reduced pressure to afford the product. The residue obtained was dried at high vacuum for 1 h and is used as such in subsequent reactions. ¹H NMR (400 MHz, Chloroform-d) δ 3.47 (dd, J=6.3, 1.9 Hz, 2H), 2.08-1.77 (m, 5H), 1.62-1.18 (m, 4H), 0.99 (qd, J=13.0, 3.2 Hz, 2H). ¹⁹F NMR (376 MHz, Chloroform-d) δ −74.33 (d, J=8.2 Hz).

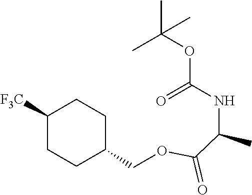 [SMILES: C[C@H](NC(=O)OC(C)(C)C)C(=O)OC[C@H]1CC[C@H](C(F)(F)F)CC1]

((1r, 4S)-4-(trifluoromethyl)cyclohexyl)methyl (tert-butoxycarbonyl)-L-alaninate. The product (1.48 g) was prepared in a manner similar to that described for Intermediate 12. ¹H NMR (400 MHz, Chloroform-d) δ 5.00 (s, 1H), 4.30 (s, 1H), 4.04-3.89 (m, 2H), 2.08-1.79 (m, 5H), 1.74-1.57 (m, 1H), 1.44 (s, 9H), 1.38 (d, J=7.2 Hz, 3H), 1.30 (m, 2H), 1.12-0.93 (m, 2H). ¹⁹F NMR (376 MHz, Chloroform-d) δ −74.38 (d, J=7.8 Hz).

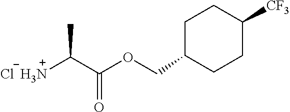 [SMILES: [Cl-] ; C[C@H]([NH3+])C(=O)OC[C@H]1CC[C@H](C(F)(F)F)CC1]

(S)-1-oxo-1-(((1r, 4S)-4-(trifluoromethyl)cyclohexyl)methoxy)propan-2-aminium chloride. The product (1.184 g) was prepared in a manner similar to that described for Intermediate 13. ¹H NMR (400 MHz, DMSO-d6) δ 8.55 (s, 3H), 4.17-3.88 (m, 3H), 2.21 (dtd, J=12.2, 8.8, 3.3 Hz, 1H), 1.83 (ddd, J=29.5, 13.4, 3.4 Hz, 4H), 1.63 (tdd, J=11.9, 6.0, 3.3 Hz, 1H), 1.41 (d, J=7.2 Hz, 3H), 1.32-0.93 (m, 4H). ¹⁹F NMR (377 MHz, DMSO-d6) δ −72.84 (d, J=8.8 Hz).

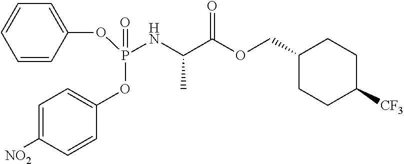 [SMILES: C[C@H](NP(=O)(Oc1ccccc1)Oc1ccc([N+](=O)[O-])cc1)C(=O)OC[C@H]1CC[C@H](C(F)(F)F)CC1]

((1r, 4S)-4-(trifluoromethyl)cyclohexyl)methyl ((4-nitrophenoxy)(phenoxy)phosphoryl)-L-alaninate. The product (1.4 g) was prepared in a manner similar to that described for Intermediate 35. ¹H NMR (400 MHz, DMSO-d6) δ 8.37-8.22 (m, 2H), 7.56-7.31 (m, 4H), 7.30-7.14 (m, 2H), 6.72 (ddd, J=13.7, 10.1, 8.6 Hz, 1H), 4.10-3.91 (m, 1H), 3.88-3.75 (m, 2H), 2.20-1.99 (m, 1H), 1.86-1.63 (m, 4H), 1.54-1.41 (m, 1H), 1.29-1.06 (m, 5H), 0.98 (td, J=12.7, 3.2 Hz, 2H). MS m/z=531.02 [M+1].

### Intermediate 34. Ethyl ((S)-(perfluorophenoxy)(phenoxy)phosphoryl)-L-alaninate

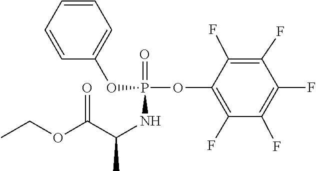 [SMILES: CCOC(=O)[C@H](C)N[P@](=O)(Oc1ccccc1)Oc1c(F)c(F)c(F)c(F)c1F]

To a solution of L-alanine ethyl ester-HCl (631 mg, 2.465 mmol) in DCM (15 mL) was added phenyl phosphorodichloridate (0.368 mL, 2.465 mmol) in one portion at −78° C. and triethylamine (0.68 mL, 4.93 mmol) was added dropwise over 5 min at −78° C. The resulting mixture was stirred for 30 min after removal of dry ice bath and then cooled to −78° C. Pentafluorophenol (454 mg, 2.465 mmol) was added in one portion and triethylamine (0.34 mL, 2.465 mmol) added over 5 min at −78° C. The resulting mixture was stirred for 1 h after removal of dry ice bath, then diluted with DCM, washed with brine, concentrated in vacuo, and the resulting residue purified by silica gel column chromatography (EtOAc 0 to 60% in hexanes) to give a diastereomeric mixture, to which diisopropyl ether (4 mL) was added. The suspension was sonicated and filtered. ¹H NMR of the filter cake showed it is 3:1 ratio of mixture. Diisopropyl ether (5 mL) was added to the filter cake and the suspension was heated at 70° C. to a clear solution. Upon removal of heating bath, needle like crystals started to form and after 10 min, the mixture was filtered and the filter cake was dried under high vacuum for 30 min to afford the Sp isomer.

Diastereomeric mixture: ¹H NMR (400 MHz, Chloroform-d) δ 7.43-7.30 (m, 2H), 7.32-7.17 (m, 3H), 4.29-4.11 (m, 3H), 3.94 (m, 1H), 1.52-1.42 (m, 3H), 1.28 (q, J=7.0 Hz, 3H).

Sp isomer: ¹H NMR (400 MHz, Acetonitrile-d3) δ 7.50-7.36 (m, 2H), 7.32-7.21 (m, 3H), 4.75 (t, J=11.5 Hz, 1H), 4.17-3.98 (m, 3H), 1.37 (dd, J=7.1, 1.1 Hz, 3H), 1.22 (t, J=7.1 Hz, 3H). ³¹P NMR (162 MHz, Acetonitrile-d3) δ −0.51. ¹⁹F NMR (376 MHz, Acetonitrile-d3) δ −155.48-−155.76 (m), −162.73 (td, J=21.3, 3.7 Hz), −165.02-−165.84 (m). LCMS m/z=440.5 (M-ethyl+H), t_{R}=1.57 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 35. (2S)-2-ethylbutyl 2-cyclohexyl-2-(((4-nitrophenoxy) (phenoxy)phosphoryl)amino)acetate

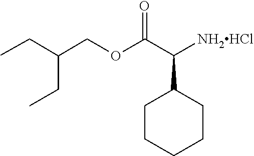

(S)-2-ethylbutyl 2-amino-2-cyclohexylacetate hydrochloride. Took up L-cyclohexylglycine (0.90 g, 5.75 mmol) in 2-ethyl-1-butanol (20 mL) and added chlorotimethylsilane (1.31 mL, 10.30 mmol) in one portion. Placed in a preheated 60° C. oil bath for 16 h. Concentrated and co-evaporated with toluene 5 times in a 60° C. rotary evaporator bath. Placed under high vacuum overnight to afford the product. The material was used as is for the next step. ¹H NMR (400 MHz, DMSO-d₆) δ 8.38 (s, 3H), 4.17-3.96 (m, 2H), 3.84 (d, J=4.5 Hz, 1H), 1.90-1.40 (m, 5H), 1.41-0.88 (m, 11H), 0.83 (t, J=7.3 Hz, 6H).

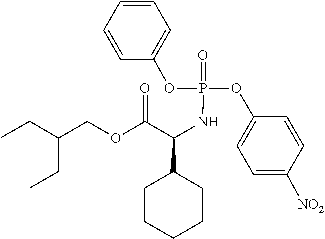 [SMILES: CCC(CC)COC(=O)[C@@H](NP(=O)(Oc1ccccc1)Oc1ccc([N+](=O)[O-])cc1)C1CCCCC1]

(2S)-2-ethylbutyl 2-cyclohexyl-2-(((4-nitrophenoxy) (phenoxy)phosphoryl)amino)acetate. To a solution of (S)-2-ethylbutyl 2-amino-2-cyclohexylacetate hydrochloride (1.50 g, 5.39 mmol) and phenyl dichlorophosphate (0.803 mL, 5.39 mmol) in dichloromethane (50 mL) was added triethylamine (1.56 mL, 11.16 mmol) at 0° C. under an argon atmosphere. The resulting mixture was allowed to warm to RT and was stirred for 1 h. 4-Nitrophenol (713 mg, 5.13 mmol) and triethylamine (0.81 mL, 5.63 mmol) were then added. After 2 h, the reaction mixture was diluted with Et₂O (100 mL) and the solids were filtered off. The crude was concentrated under reduced pressure and was purified by silica gel chromatography (120 g SiO₂ Combiflash HP Gold Column, 0-50% ethyl acetate/hexanes), followed by purification by reverse phase HPLC without modifier 20-100% ACN in Water to afford the product. ¹H NMR (400 MHz, DMSO-d₆) δ 8.28 (br d, J=9.3 Hz, 2H), 7.55-7.28 (m, 4H), 7.28-7.01 (m, 3H), 6.61-6.52 (m, 1H), 3.85 (d, J=4.0 Hz, 2H) 3.75-3.53 (m, 1H), 1.67-1.31 (m, 7H), 1.25 (m, 6H), 1.16-0.67 (m, 9H). LC/MS: t_{R}=1.48 min, MS m/z=519.03 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

### Intermediate 36. (1-(2,2,2-trifluoroethyl)piperidin-4-yl)methyl ((4-nitrophenoxy)(phenoxy)phosphoryl)alaninate

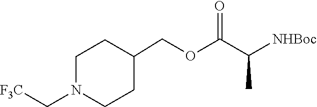 [SMILES: C[C@H](NC(=O)OC(C)(C)C)C(=O)OCC1CCN(CC(F)(F)F)CC1]

(1-(2,2,2-trifluoroethyl)piperidin-4-yl)methyl (tert-butoxycarbonyl)alaninate. The product (3.8 g) was prepared in a manner similar to that described for Intermediate 12. ¹H NMR (400 MHz, DMSO-d₆) δ 7.25 (d, J=7.4 Hz, 1H), 4.08-3.72 (m, 3H), 3.10 (q, J=10.3 Hz, 2H), 2.88 (d, J=11.0 Hz, 2H), 2.37-2.18 (m, 2H), 1.66-1.47 (m, 3H), 1.36 (s, 9H), 1.21 (d, J=7.5 Hz, 5H). ¹⁹F NMR (376 MHz, DMSO-d₆) δ −68.52 (t, J=10.3 Hz).

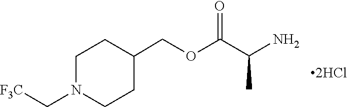 [SMILES: C[C@H](N)C(=O)OCC1CCN(CC(F)(F)F)CC1]

(1-(2,2,2-trifluoroethyl)piperidin-4-yl)methyl alaninate dihydrochloride. The product (3.52 g) was prepared in a manner similar to that described for Intermediate 13. ¹H NMR (400 MHz, DMSO-d₆) δ 8.67 (s, 3H), 4.44-3.75 (m, 5H), 3.49-2.81 (m, 4H), 2.00-1.61 (m, 5H), 1.43 (d, J=7.2 Hz, 3H). ¹⁹F NMR (376 MHz, DMSO-d₆) δ −63.30 (d, J=443.2 Hz).

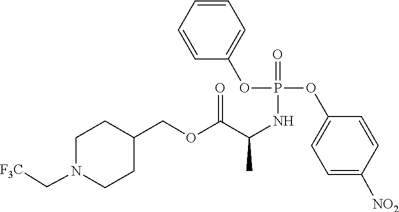 [SMILES: C[C@H](NP(=O)(Oc1ccccc1)Oc1ccc([N+](=O)[O-])cc1)C(=O)OCC1CCN(CC(F)(F)F)CC1]

(1-(2,2,2-trifluoroethyl)piperidin-4-yl)methyl ((4-nitrophenoxy)(phenoxy)phosphoryl)alaninate. The product (4.25 g) was prepared in a manner similar to that described for Intermediate 35. ¹H NMR (400 MHz, DMSO-d₆) δ 8.32-8.24 (m, 2H), 7.53-7.40 (m, 2H), 7.39 (ddd, J=8.1, 6.8, 3.1 Hz, 2H), 7.24 (ddd, J=17.4, 6.5, 1.6 Hz, 3H), 6.69 (ddd, J=13.7, 10.0, 8.4 Hz, 1H), 4.07-3.92 (m, 1H), 3.88-3.77 (m, 2H), 3.08 (qd, J=10.3, 1.6 Hz, 2H), 2.87-2.79 (m, 2H), 2.25-2.14 (m, 2H), 1.56-1.39 (m, 3H), 1.26-1.08 (m, 5H). ³¹P NMR (162 MHz, DMSO-d₆) δ −1.26, −1.49. ¹⁹F NMR (376 MHz, DMSO-d₆) δ −68.45 (td, J=10.2, 2.4 Hz). LCMS: MS m/z=546.27 [M+1];]; t_{R}=1.12 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 37. (1-Ethyl-3,3-difluoropiperidin-4-yl ((4-nitrophenoxy)(phenoxy)phosphoryl)-L-alaninate

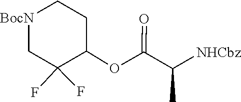 [SMILES: C[C@H](NC(=O)OCc1ccccc1)C(=O)OC1CCN(C(=O)OC(C)(C)C)CC1(F)F]

tert-Butyl 4-((((benzyloxy)carbonyl)-L-alanyl)oxy)-3,3-difluoropiperidine-1-carboxylate. To a mixture of N-Cbz-L-alanine (2.0 g, 8.96 mmol), tert-butyl 3,3-difluoro-4-hydroxypiperidine-1-carboxylate (2.12 g, 8.96 mmol), and EDCI (1.67 g, 10.75 mmol) in acetonitrile (20 mL) was added DMAP (1.64 g, 13.44 mmol). Then the mixture was stirred at room temperature for 15 h, then diluted with EtOAc, washed with brine, dried over sodium sulfate, and concentrated in vacuo. The obtained residue was purified by silica gel chromatography (EtOAc 50 to 100% in hexanes) to afford the product. ¹⁹F NMR (377 MHz, Chloroform-d) δ −114.32 (m), −117.73-−121.11 (m). LCMS: MS m/z=343.14 [M+1-Boc], 386.82 (M+1-t-Bu); t_{R}=1.23 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

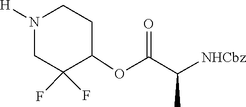 [SMILES: C[C@H](NC(=O)OCc1ccccc1)C(=O)OC1CCNCC1(F)F]

3,3-difluoropiperidin-4-yl ((benzyloxy)carbonyl)-L-alaninate. To a mixture of tert-Butyl 4-((((benzyloxy)carbonyl)-L-alanyl)oxy)-3,3-difluoropiperidine-1-carboxylate (330 mg, 0.746 mmol) in DCM (5 mL) was added 4 M HCL in dioxane (0.9 mL) slowly at room temperature. The resulting mixture was stirred at room temperature for 2 h, concentrated in vacuo, co-evaporation with DCM several times, and dried under high vacuum for 15 h to afford the product. ¹H NMR (400 MHz, Chloroform-d) δ 7.33 (m, 5H), 5.59 (m, 1H), 5.27-5.01 (m, 3H), 4.53-4.25 (m, 1H), 3.12 (m, 1H), 3.03-2.76 (m, 2H), 2.73 (s, 1H), 1.94 (s, 1H), 1.80 (s, 1H), 1.41 (d, J=7.2 Hz, 3H). ¹⁹F NMR (376 MHz, Chloroform-d) δ −114.66 (dd, J=245.9, 61.8 Hz), −119.63.

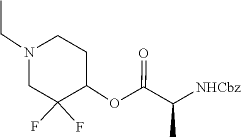 [SMILES: CCN1CCC(OC(=O)[C@H](C)NC(=O)OCc2ccccc2)C(F)(F)C1]

1-ethyl-3,3-difluoropiperidin-4-yl ((benzyloxy)carbonyl)-L-alaninate. A mixture of 3,3-difluoropiperidin-4-yl ((benzyloxy)carbonyl)-L-alaninate (450 mg, 1.190 mmol), acetaldehyde (0.194 mL, 2.629 mmol), and acetic acid (0.15 mL, 2.629 mmol) in DCM (9 mL) was stirred for 20 min at room temperature and sodium cyanoborohydride (330 mg, 5.258 mmol) was added. The resulting mixture was stirred for 1 h and purified by preparative HPLC (Phenominex Gemini 10 u C18 110 Å 250×21.2 mm column, 20-80% acetonitrile (0.1% TFA)/water (0.1% TFA) gradient) to afford the product. ¹H NMR (400 MHz, Acetonitrile-d₃) δ 10.18 (bs, 2H), 7.38 (m, 5H), 6.19 (m, 1H), 5.47-5.26 (m, 1H), 4.33 (m, 1H), 3.82-2.98 (m, 6H), 2.30 (s, 1H), 2.16 (s, 1H), 1.42 (m, 3H), 1.31 (td, J=7.3, 1.5 Hz, 3H). LCMS: MS m/z=371.27 [M+1]; t_{R}=0.66 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

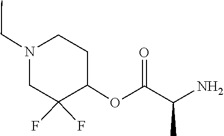 [SMILES: CCN1CCC(OC(=O)[C@H](C)N)C(F)(F)C1]

1-ethyl-3,3-difluoropiperidin-4-yl L-alaninate. A mixture of 1-ethyl-3,3-difluoropiperidin-4-yl ((benzyloxy)carbonyl)-L-alaninate (450 mg, 0.929 mmol) and 20% Pd(OH)₂/C in THF (10 mL) was stirred at room temperature under H₂ gas for 1 h, filtered, concentrated in vacuo, co-evaporated with DCM several time, and dried under high vacuum for 1 h to afford the product. LCMS: MS m/z=237.09 [M+1]; t_{R}=0.15 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

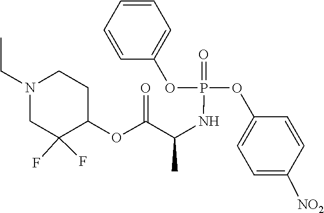 [SMILES: CCN1CCC(OC(=O)[C@H](C)NP(=O)(Oc2ccccc2)Oc2ccc([N+](=O)[O-])cc2)C(F)(F)C1]

(1-Ethyl-3,3-difluoropiperidin-4-yl ((4-nitrophenoxy)(phenoxy)phosphoryl)-L-alaninate. Methylene chloride (10 mL) was added to the syrup of 1-ethyl-3,3-difluoropiperidin-4-yl L-alaninate (480 mg, 1.37 mmol) and TEA (0.190 mL, 0.370 mmol) was added to achieve a solution, which was cooled to −78° C. and phenyl dichlorophosphate (0.205 mL, 1.370 mmol) was added quickly. Triethylamine (0.190 mL, 1.37 mmol) was added over 30 min at −78° C. The resulting mixture was stirred for 30 min at the same temperature and 4-nitrophenol (191 mg, 1.370 mmol) added in one portion. Then triethylamine (0.190 mL, 1.370 mmol) was added over 30 min at −78° C. Then the mixture was stirred for 2 h at room temperature, washed with water and brine, dried over sodium sulfate, and concentrated in vacuo. The residue was then purified by silica gel column chromatography (EtOAc 0 to 100% in hexanes) to give the product. ¹H NMR (400 MHz, Chloroform-d) δ 8.29-8.15 (m, 2H), 7.44-7.28 (m, 4H), 7.27-7.11 (m, 3H), 5.03 (m, 1H), 4.34-4.14 (m, 1H), 3.94-3.75 (m, 1H), 2.88 (s, 1H), 2.63-2.49 (m, 4H), 2.39 (m, 1H), 2.03-1.93 (m, 1H), 1.93-1.77 (m, 1H), 1.44 (m, 3H), 1.09 (td, J=7.2, 1.0 Hz, 3H). ³¹P NMR (162 MHz, Chloroform-d) δ −3.21, −3.26, −3.32, −3.46. ¹⁹F NMR (377 MHz, Chloroform-d) δ −110.50 (d, J=244.0 Hz), −116.76 (m). LCMS: MS m/z=514.29 [M+1]; t_{R}=0.80 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 38. 4-nitrophenyl-N,N′-ethyl L-alaninatephosphorodiamidate

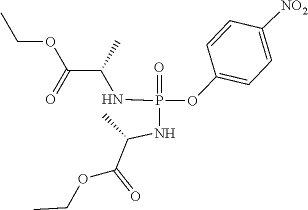 [SMILES: CCOC(=O)[C@H](C)NP(=O)(N[C@@H](C)C(=O)OCC)Oc1ccc([N+](=O)[O-])cc1]

To a solution of ethyl L-alaninate HCl salt (1.8 g, 11.72 mmol) in DCM (20 mL) was added 4-nitrophenyl phosphorodichloridate (1.5 g, 5.86 mmol) in one portion. The resulting mixture was cooled to 0° C. and triethylamine (2.37 g, 23.44 mmol) was added dropwise. The resulting mixture was stirred for 30 min after removal of ice bath and was stirred for overnight. The reaction mixture was then diluted with EtOAc, washed with water and brine, the organic solvent was concentrated in vacuum, and the resulting residue was purified by silica gel column chromatography eluting with 0-100% ethyl acetate in hexanes to afford the product. LCMS: MS m/z=417.93 [M+1], t_{R}=1.23 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.02 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 39. benzyl ((4-nitrophenoxy)(phenoxy)phosphoryl)-L-alaninate

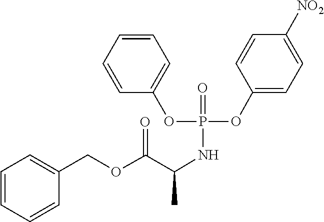 [SMILES: C[C@H](NP(=O)(Oc1ccccc1)Oc1ccc([N+](=O)[O-])cc1)C(=O)OCc1ccccc1]

Phenyl dichlorophosphate (1.49 mL, 10 mmol) was dissolved in 20 mL anhydrous dichloromethane and stirred under atmospheric nitrogen in an ice bath. L-Alanine benzyl ester HCl (2.2 g, 10 mmol) was added to the reaction solution in one portion and stirred for 10 min. Triethylamine (3 mL, 22 mmol) was dissolved in 5 mL of anhydrous dichloromethane and added to the reaction dropwise. The reaction mixture was stirred for 2 h. p-Nitrophenol (1.25 g, 9 mmol) was added in one portion. Triethylamine (1.5 mL, 11 mmol) was dissolved in 3 mL of anhydrous dichloromethane and added to the reaction dropwise. The reaction mixture was stirred for 1 h, and was diluted with dichloromethane (10 mL) and washed with water (3×10 mL). Organic extract was dried over anhydrous sodium sulfate and concentrated under reduced pressure. The crude residue was purified via SiO₂ column chromatography (4 g SiO₂ Combiflash HP Gold Column, 0-30% ethyl acetate/hexanes). Fractions containing the desired product were combined and concentrated under reduced pressure to afford the product. ¹H NMR (400 MHz, chloroform-d) δ 8.24-8.10 (m, 2H), 7.40-7.10 (m, 12H), 5.14 (m, 2H), 4.19 (m, 1H), 3.87 (m, 1H), 1.47-1.36 (m, 3H). ³¹P NMR (162 MHz, chloroform-d) δ −3.15, −3.29. LCMS: MS m/z=457.1 [M+1]; 455.1 [M−1], t_{R}=1.45 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=4.03 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 40. 4-nitrophenyl-N,N′-methyl L-alaninatephosphorodiamidate

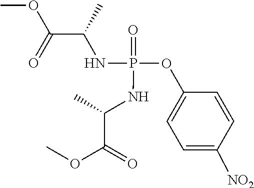 [SMILES: COC(=O)[C@H](C)NP(=O)(N[C@@H](C)C(=O)OC)Oc1ccc([N+](=O)[O-])cc1]

Triethylamine (3.68 mL, 26.4 mmol) was added to a solution of methyl L-alaninate hydrochloride (1.63 g, 12.0 mmol) and 4-nitrophenyl phosphorodichloridate (1.5 g, 5.9 mmol) in dichloromethane (23 mL) at 0° C. under an argon atmosphere. After 3 h, the reaction mixture was diluted with dichloromethane (50 mL), washed with saturated aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, chloroform-d₁) 8.25-8.16 (m, 2H), 7.38 (dd, J=9.3, 1.0 Hz, 2H), 4.17-3.95 (m, 2H), 3.73 (br s, 6H), 3.61 (br t, J=10.0 Hz, 2H), 1.42 (s, 3H), 1.40 (s, 1H). ³¹P NMR (162 MHz, chloroform-d₁) δ 7.82 (s). LCMS: MS m/z=389.98 [M+1], t_{R}=1.11 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.81 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

### Intermediate 41. methyl ((4-nitrophenoxy)(phenoxy)phosphoryl)-L-alaninate

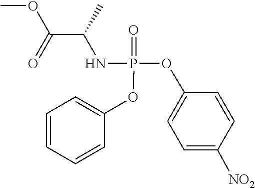 [SMILES: COC(=O)[C@H](C)NP(=O)(Oc1ccccc1)Oc1ccc([N+](=O)[O-])cc1]

Phenyl dichlorophosphate (2.81 mL, 18.9 mmol) and triethylamine (5.38 mL, 37.9 mmol) were sequentially added to a suspension of methyl L-alaninate hydrochloride (2.64 g, 18.9 mmol) in dichloromethane (100 mL) at 0° C. After 1 h, 4-nitrophenol (2.64 g, 18.9 mmol) and triethylamine (2.64 mL, 18.9 mmol) were then sequentially added at 0° C., and the resulting mixture was then allowed to warm to RT. After 2.5 h, the reaction mixture was diluted with dichloromethane (100 mL), washed with saturated a aqueous sodium bicarbonate solution (100 mL) and brine (100 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, chloroform-d₁) δ 8.25-8.18 (m, 2H), 7.43-7.29 (m, 4H), 7.29-7.15 (m, 3H), 4.24-4.07 (m, 1H), 3.97 (br q, J=9.8 Hz, 1H), 3.70 (s, 3H), 1.45-1.35 (m, 3H). ³¹P NMR (162 MHz, chloroform-d₁) δ −3.12 (s), −3.17 (s). LCMS: MS m/z=380.98 [M+1], t_{R}=1.59 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.49 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

### Intermediate 42. methyl ((4-nitrophenoxy)(phenoxy)phosphoryl)-L-alaninate

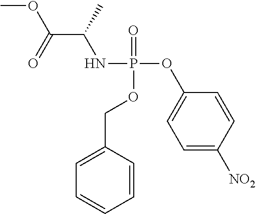 [SMILES: COC(=O)[C@H](C)NP(=O)(OCc1ccccc1)Oc1ccc([N+](=O)[O-])cc1]

4-Nitrophenyl phosphorodichloridate (2.00 g, 7.81 mmol) and triethylamine (2.18 mL, 15.6 mmol) were sequentially added to a suspension of methyl L-alaninate hydrochloride (1.091 g, 18.9 mmol) in dichloromethane (23 mL) at 0° C. under an argon atmosphere. After 1 h, benzyl alcohol (0.810 mL, 7.81 mmol) and triethylamine (1.09 mL, 7.81 mmol) were then sequentially added at 0° C., and the resulting mixture was then allowed to warm to RT. After 1 h, the reaction mixture was diluted with dichloromethane (50 mL), washed with saturated an aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, chloroform-d) δ 8.32-8.09 (m, 2H), 8.32-8.09 (m, 7H), 5.15 (app t, J=8.4 Hz, 2H), 4.70 (s, 1H), 4.07-3.93 (m, 1H), 3.73-3.65 (m, 3H), 1.42-1.31 (m, 3H). ³¹P NMR (162 MHz, chloroform-d₁) δ 2.23 (s), 2.15 (s). LCMS: MS m/z=394.9[M+1], t_{R}=1.34 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

### Intermediate 43. Isopropyl ((4-(dimethylcarbamoyl)phenoxy)(4-nitrophenoxy)phosphoryl)-L-alaninate

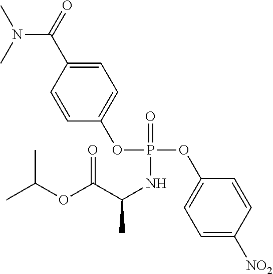 [SMILES: CC(C)OC(=O)[C@H](C)NP(=O)(Oc1ccc(C(=O)N(C)C)cc1)Oc1ccc([N+](=O)[O-])cc1]

To a solution of 4-nitrophenyl phosphorodichloridate (620 mg, 2.422 mmol) and isopropyl L-alanine-HCl (406 mg, 2.422 mmol) in DCM-THF (10:3 mL) was added TEA (0.68 mL, 4.844 mmol) in DCM (3.32 mL) over 30 min at −78° C. The resulting mixture was stirred for 30 min after removal of dry ice bath and cooled to −78° C. and N,N-dimethyl-4-hydroxybenzamide (400 mg, 2.422 mmol) was added in one portion and TEA (0.34 mL, 2.422 mmol) in DCM (3.66 mL) added over 30 min at −78° C. The resulting mixture was stirred for 1 h after removal of dry ice bath, then diluted with EtOAc, washed with brine, concentrated in vacuo, and the resulting residue purified by silica gel column chromatography (EtOAc 0 to 100% in hexanes) to afford the product. ¹H NMR (400 MHz, Chloroform-d) δ 8.26-8.18 (m, 2H), 7.45-7.35 (m, 3H), 7.27 (m, 2H), 6.76 (m, 1H), 5.01 (m, 1H), 4.17-3.94 (m, 2H), 3.19-2.84 (m, 6H), 1.39 (m, 3H), 1.27-1.16 (m, 6H). ³¹P NMR (162 MHz, Chloroform-d) δ −3.13, −3.21. MS m/z=480 (M+H). LCMS: MS m/z=480.26 [M+1]; t_{R}=1.00 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 44. oxetan-3-yl ((4-nitrophenoxy)(phenoxy)phosphoryl)-L-alaninate

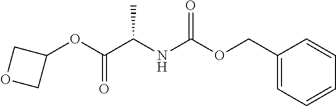 [SMILES: C[C@H](NC(=O)OCc1ccccc1)C(=O)OC1COC1]

oxetan-3-yl ((benzyloxy)carbonyl)-L-alaninate. To a mixture of ((benzyloxy)carbonyl)-L-alanine (1.8 g, 8.1 mmol), 3-hydroxyoxetane (0.5 g, 6.75 mmol) and 1-Ethyl-3-(3-dimethylaminopropyl)carbodiimide HCl salt (EDCI) (1.68 g, 8.77 mmol) in acetonitrile (100 mL) was added 4-(Dimethylamino)pyridine (DMAP, 1.24 g, 10.12 mmol). Then the mixture was stirred at room temperature for 2 h, then the reaction mixture was diluted with EtOAc, washed with brine, dried organic solvent over sodium sulfate, and then concentrated in vacuum. The obtained residue was purified by silica gel chromatography eluting with 0-100% ethyl acetate in hexanes to afford the product. ¹H NMR (400 MHz, Chloroform-d) δ 7.40-7.28 (m, 5H), 5.47 (p, J=5.9 Hz, 1H), 5.30 (d, J=8.0 Hz, 1H), 5.10 (s, 2H), 4.88 (t, J=7.1 Hz, 2H), 4.62 (ddd, J=17.5, 7.7, 5.3 Hz, 2H), 4.41 (p, J=7.3 Hz, 1H), 1.44 (d, J=7.3 Hz, 3H). LCMS: MS m/z=280.04 [M+1], t_{R}=1.11 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.82 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

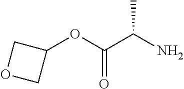

oxetan-3-yl L-alaninate. Dissolved oxetan-3-yl ((benzyloxy)carbonyl)-L-alaninate (0.1 g, 0.36 mmol) in DCM (5 mL), to the solution was added 15 mg of Pd—C(10%, wet), the reaction flask was degassed and then charged with H₂ balloon, stirred at RT for 2 h, the reaction mixture was then filtered, solvent was evaporated under vacuum, the residue was dried on high vacuum for 5 min to afford the product. ¹H NMR (400 MHz, Chloroform-d) δ 5.42 (p, J=5.7 Hz, 1H), 4.87 (t, J=6.9 Hz, 2H), 4.65-4.54 (m, 2H), 3.58 (qd, J=7.0, 2.1 Hz, 1H), 1.49 (d, J=7.1 Hz, 2H), 1.34 (dd, J=7.2, 2.1 Hz, 3H).

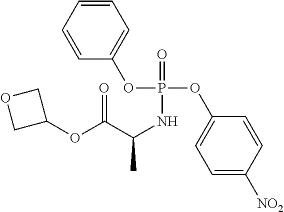 [SMILES: C[C@H](NP(=O)(Oc1ccccc1)Oc1ccc([N+](=O)[O-])cc1)C(=O)OC1COC1]

oxetan-3-yl ((4-nitrophenoxy)(phenoxy)phosphoryl)-L-alaninate. To a solution of oxetan-3-yl L-alaninate (120 mg, 0.83 mmol) in DCM (10 mL) was added phenyl phosphorodichloridate (175 mg, 0.83 mmol) in one portion. The resulting mixture was cooled to 0° C. and triethylamine (252 mg, 2.49 mmol) was added dropwise. The resulting mixture was stirred for 30 min after removal of ice bath and cooled to 0° C. and para-nitrophenol (115 mg, 0.83 mmol) was added in one portion and triethylamine (252 mg, 2.49 mmol) was added dropwise. The resulting mixture was stirred for 30 min after removal of ice bath, diluted with EtOAc, washed with water and brine, the organic solvent was concentrated in vacuum, and the resulting residue was purified by silica gel column chromatography eluting with 0-100% ethyl acetate in hexanes to afford the product. LCMS: MS m/z=423.06 [M+1], t_{R}=1.25 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.15 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 45. propyl ((4-nitrophenoxy)(phenoxy)phosphoryl)-L-alaninate

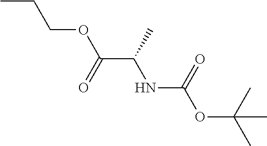 [SMILES: CCCOC(=O)[C@H](C)NC(=O)OC(C)(C)C]

propyl (tert-butoxycarbonyl)-L-alaninate. N-(3-Dimethylaminopropyl)-N′-ethylcarbodiimide hydrochloride (6.08 g, 31.71 mmol) was added to a solution of Boc-Ala-OH (5 g, 26.43 mmol) and n-propyl alcohol (6.02 mL, 80.6 mmol) in acetonitrile (125 mL) at RT. After 15 min, 4-(dimethylamino)pyridine (3.23 g, 26.43 mmol) was added. After 16 h, the reaction mixture was concentrated to half the volume, and the mixture was diluted with ethyl acetate (250 mL) and the resulting mixture was washed with saturated aqueous sodium carbonate solution (2×200 mL) and brine (200 mL). The organic layer was dried over anhydrous sodium sulfate and concentrated under reduced pressure. The crude residue was subjected to silica gel chromatography eluting with 0-20% EtOAc in hexane to afford the product. ¹H NMR (400 MHz, Acetonitrile-d₃) δ 5.57 (s, 1H), 4.19-3.92 (m, 3H), 1.63 (h, J=7.1 Hz, 2H), 1.40 (s, 9H), 1.30 (d, J=7.3 Hz, 3H), 0.93 (t, J=7.4 Hz, 3H). LCMS: MS m/z=231.60 [M+1], t_{R}=1.10 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

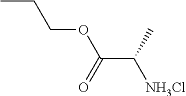

propyl L-alaninate hydrochloride. 4 M Hydrochloric acid solution in dioxane (16.91 mL) was added to propyl (tert-butoxycarbonyl)-L-alaninate (3.91 g, 16.91 mmol) in dichloromethane (10 mL) at RT. After 16 h, reaction mixture was concentrated under reduced pressure to afford the product. ¹H NMR (400 MHz, Acetonitrile-d₃) δ 8.45 (s, 3H), 4.22-4.11 (m, 2H), 4.11-3.99 (m, 1H), 1.68 (dtd, J=14.0, 7.4, 6.6 Hz, 2H), 1.60 (d, J=7.2 Hz, 3H), 0.95 (t, J=7.4 Hz, 3H). LCMS: MS m/z=131.94 [M+1], t_{R}=0.32 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

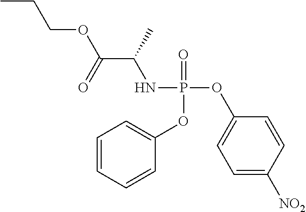 [SMILES: CCCOC(=O)[C@H](C)NP(=O)(Oc1ccccc1)Oc1ccc([N+](=O)[O-])cc1]

propyl ((4-nitrophenoxy)(phenoxy)phosphoryl)-L-alaninate. Phenyl dichlorophosphate (0.89 mL, 5.97 mmol) in dichloromethane (12 mL) was added dropwise over 15 minutes to a solution of propyl L-alaninate hydrochloride (1.0 g, 5.97 mmol) in dichloromethane (12 mL) at 0° C. After the addition was complete, triethylamine (2.0 mL, 14.32 mmol) in dichloromethane (2.5 mL) was added over 5 minutes. After 3.5 h, 4-nitrophenol (0.83 g, 5.97 mmol) and triethylamine (1.0 mL, 7.16 mmol) were then sequentially added at 0° C., and the resulting mixture was then allowed to warm to RT. After 2 h, the reaction mixture was diluted with dichloromethane (50 mL), washed with water (2×100 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, Acetonitrile-d₃) δ 8.28-8.20 (m, 2H), 7.49-7.35 (m, 4H), 7.31-7.19 (m, 3H), 4.72-4.56 (m, 1H), 4.14-4.02 (m, 1H), 3.99 (td, J=6.6, 2.5 Hz, 2H), 1.58 (dtdd, J=13.9, 7.4, 6.5, 0.9 Hz, 2H), 1.31 (ddd, J=7.1, 4.2, 1.1 Hz, 3H), 0.88 (t, J=7.4 Hz, 3H). ³¹P NMR (162 MHz, Acetonitrile-d₃) δ −2.12, −2.22. LCMS: MS m/z=409.12 [M+1], t_{R}=1.15 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.73 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 46. oxetan-3-ylmethyl ((4-nitrophenoxy)(phenoxy)phosphoryl)-L-alaninate

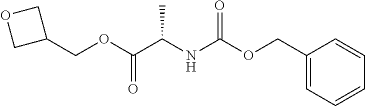 [SMILES: C[C@H](NC(=O)OCc1ccccc1)C(=O)OCC1COC1]

oxetan-3-ylmethyl ((benzyloxy)carbonyl)-L-alaninate. To a mixture of ((benzyloxy)carbonyl)-L-alanine (6.08 g, 27.24 mmol), oxetan-3-ylmethanol (2 g, 22.7 mmol) and 1-Ethyl-3-(3-dimethylaminopropyl)carbodiimide HCl salt (EDCI) (5.66 g, 29.51 mmol) in acetonitrile (100 mL) was added 4-(Dimethylamino)pyridine (DMAP, 4.16 g, 34.05 mmol). Then the mixture was stirred at room temperature for 2 h, the reaction mixture was then diluted with EtOAc, washed with brine, dried organic solvent over sodium sulfate, and then concentrated in vacuum. The obtained residue was purified by silica gel chromatography eluting with 0-100% ethyl acetate in hexanes to afford the product. LCMS: MS m/z=280.04 [M+1], t_{R}=1.11 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.88 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

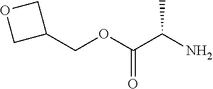

oxetan-3-ylmethyl L-alaninate. Dissolved oxetan-3-ylmethyl ((benzyloxy)carbonyl)-L-alaninate (2.2 g, 8 mmol) in DCM (25 mL), to the solution was added 500 mg of Pd—C(10%, wet), the reaction flask was degassed and then charged with H₂ balloon, stirred at RT for 2 h, the reaction mixture was then filtered, solvent was evaporated under vacuum, the residue was dried on high vacuum for 5 min to afford the product. ¹H NMR (400 MHz, Chloroform-d) δ 4.77 (dd, J=7.9, 6.3 Hz, 2H), 4.44 (td, J=6.1, 2.5 Hz, 2H), 4.38-4.23 (m, 2H), 3.55 (q, J=7.0 Hz, 1H), 3.34-3.19 (m, 1H), 1.31 (d, J=7.0 Hz, 3H).

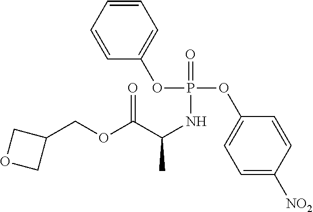 [SMILES: C[C@H](NP(=O)(Oc1ccccc1)Oc1ccc([N+](=O)[O-])cc1)C(=O)OCC1COC1]

oxetan-3-ylmethyl ((4-nitrophenoxy)(phenoxy)phosphoryl)-L-alaninate. To a solution of oxetan-3-ylmethyl L-alaninate (1.19 g, 7.11 mmol) in DCM (20 mL) was added phenyl phosphorodichloridate (1.5 g, 7.11 mmol) in one portion. The resulting mixture was cooled to 0° C. and triethylamine (1.44 g, 14.22 mmol) was added drop wise. The resulting mixture was stirred for 30 min after removal of ice bath and cooled to 0° C. and para-nitrophenol (0.99 g, 7.1 mmol) was added in one portion and triethylamine (1.44 g, 14.22 mmol) was added dropwise. The resulting mixture was stirred for 30 min after removal of ice bath, diluted with EtOAc, washed with water and brine, the organic solvent was concentrated in vacuum, and the resulting residue was purified by silica gel column chromatography eluting with 0-100% ethyl acetate in hexanes to afford the product. LCMS: MS m/z=437.14 [M+1], t_{R}=1.25 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.36 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Intermediate 47. cyclobutyl ((4-nitrophenoxy)(phenoxy)phosphoryl)-L-alaninate

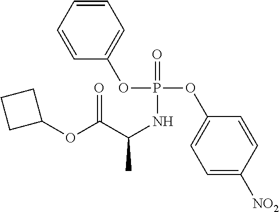 [SMILES: C[C@H](NP(=O)(Oc1ccccc1)Oc1ccc([N+](=O)[O-])cc1)C(=O)OC1CCC1]

To a solution of L-Alanine, cyclobutyl ester (1.8 g, 10 mmol) in DCM (10 mL) under a nitrogen atmosphere in an ice bath was added phenyl phosphorodichloridate (2.1 g, 10 mmol) in one portion. Then triethylamine (1.11 g, 11 mmol) was added dropwise. The resulting mixture was stirred for 2 h after removal of ice bath and cooled to 0° C. and para-nitrophenol (2.5 g, 18 mmol) was added in one portion and triethylamine (1.11 g, 11 mmol) was added dropwise. The resulting mixture was stirred for 2 h after removal of ice bath, diluted with EtOAc, washed with 5% aqueous citric acid solution twice, followed by washing with brine, the organic solvent was concentrated in vacuum, and the resulting residue was purified by silica gel column chromatography eluting with 0-100% ethyl acetate in hexanes to afford the product. MS m/z=422.0 (M+H)⁺.

Resolution of the Sp and Rp diastereomers. The product was purified via chiral preparatory HPLC (Chiralpak IA, 150×4.6 mm, Heptane 70%, IPA 30%) to form Intermediate 48 and Intermediate 49:

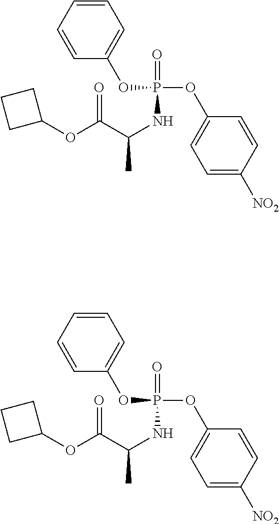 [SMILES: C[C@H](N[P@](=O)(Oc1ccccc1)Oc1ccc([N+](=O)[O-])cc1)C(=O)OC1CCC1 ; C[C@H](N[P@@](=O)(Oc1ccccc1)Oc1ccc([N+](=O)[O-])cc1)C(=O)OC1CCC1]

Intermediate 48. First Eluting Diastereomer of Intermediate 47: ¹H NMR (400 MHz, Methanol-d4) δ 8.33-8.23 (m, 2H), 7.52-7.33 (m, 4H), 7.33-7.17 (m, 3H), 4.96-4.85 (m, 1H), 4.07-3.96 (m, 1H), 2.27 (m, 2H), 2.07-1.91 (m, 2H), 1.83-1.70 (m, 1H), 1.70-1.55 (m, 1H), 1.32 (ddd, J=7.2, 5.3, 1.2 Hz, 3H). ³¹P NMR (162 MHz, Methanol-d4) δ 1.36. LCMS: MS m/z=421.05 [M+1], t_{R}=1.42 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=8.07 min; HPLC system: Chiralpak IC, 150×4.6 mm, 5 micron, CN=IC00CD-QC005, 1 CV=2.49 mL, CV #1, Col Valve: Position 3, 15 mL/15 min @ 1 mL/min. Pmax=300 bar; Solvent Valves: D: Heptane 70%, #6: IPA 30%.

Intermediate 49. Second Eluting Diastereomer of Intermediate 47: ¹H NMR (400 MHz, Methanol-d4) δ 8.33-8.23 (m, 2H), 7.52-7.33 (m, 4H), 7.33-7.17 (m, 3H), 4.96-4.85 (m, 1H), 4.07-3.96 (m, 1H), 2.27 (m, 2H), 2.07-1.91 (m, 2H), 1.83-1.70 (m, 1H), 1.70-1.55 (m, 1H), 1.32 (ddd, J=7.2, 5.3, 1.2 Hz, 3H). ³¹P NMR (162 MHz, Methanol-d4) δ 1.59. LCMS: MS m/z=420.90 [M+1], t_{R}=1.42 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=11.50 min; HPLC system: Chiralpak IC, 150×4.6 mm, 5 micron, CN=IC00CD-QC005, 1 CV=2.49 mL, CV #1, Col Valve: Position 3, 15 mL/15 min @ 1 mL/min. Pmax=300 bar; Solvent Valves: D: Heptane 70%, #6: IPA 30%.

### Intermediate 50. methyl ((S)-(perfluorophenoxy)(phenoxy)phosphoryl)-L-alaninate

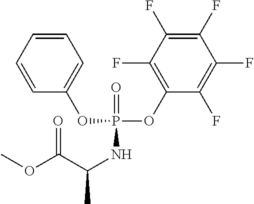 [SMILES: COC(=O)[C@H](C)N[P@](=O)(Oc1ccccc1)Oc1c(F)c(F)c(F)c(F)c1F]

L-Alanine methyl ester hydrochloride (14 g, 100 mmol) was mixed with 50 mL of anhydrous DCM and stirred under atmospheric nitrogen in an ice bath. Phenyl dichlorophosphate (16.4 mL, 110 mmol) was added to the reaction dropwise, and the reaction mixture was stirred for 30 mins. Triethylamine (29.4 mL, 210 mmol) was mixed with 20 mL anhydrous DCM and added to the reaction dropwise. Reaction was stirred for 1 hr. Pentafluorophenol (18.4 g, 100 mmol) was added in one portion. Triethylamine (14.7 mL, 105 mmol) was mixed with 30 mL of anhydrous DCM and added to reaction dropwise. The reaction mixture was stirred for 16 hrs at RT.

Reaction was diluted with DCM (50 mL) and washed with water (5×10 mL). Organic was dried over anhydrous sodium sulfate and then concentrated under reduced pressure to give solid. Isopropyl ether (130 mL) was added to solid. Big pieces of solid were broke down and then sonicated for 20 mins, after which the mixture was then stirred for 24 hrs.

Solid was collected and washed with small amount of isopropyl ether (30 mL). Solid was dried under high vacuum to give the product. ¹H NMR (400 MHz, chloroform-d) δ 7.40-7.32 (m, 2H), 7.28-7.19 (m, 3H), 4.20 (m, 1H), 3.96-3.85 (m, 1H), 3.74 (s, 3H), 1.47 (d, J=7.1 Hz, 3H). ³¹P NMR (162 MHz, chloroform-d) δ −1.62. ¹⁹F NMR (376 MHz, chloroform-d) δ −153.82 (dd, J=18.5, 2.7 Hz), −159.99 (td, J=21.8, 3.8 Hz), −162.65 (dd, J=22.2, 17.6 Hz). LCMS: MS m/z=425.9 [M+1], 423.9 [M−1], t_{R}=1.68 min; LC system: Thermo Dionex Ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.76 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 51. isopropyl ((4-(2-methoxyethoxy)phenoxy)(4-nitrophenoxy)phosphoryl)-L-alaninate

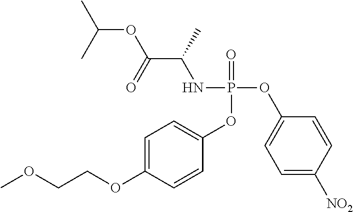 [SMILES: COCCOc1ccc(OP(=O)(N[C@@H](C)C(=O)OC(C)C)Oc2ccc([N+](=O)[O-])cc2)cc1]

4-Nitrophenyl phosphorodichloridate (503 mg, 1.97 mmol) in dichloromethane (20 mL) was added dropwise over 10 minutes to a solution of L-alanine isopropyl ester hydrochloride (329 mg, 1.97 mmol) in dichloromethane (20 mL) at 0° C. After addition was complete, triethylamine (0.55 mL, 3.93 mmol) was added dropwise. After 90 minutes, 4-(2-methoxy-ethoxy)phenol (331 mg, 1.97 mmol) and triethylamine (0.28 mL, 1.97 mmol) were sequentially added at 0° C., and the resulting mixture was then allowed to warm to RT. After 30 minutes, the reaction mixture was washed with water (2×50 mL) and brine (50 mL), dried over anhydrous sodium sulfate, and concentrated under reduced pressure. The crude residue was purified by silica gel chromatography eluting with 20-100% ethyl acetate in hexanes to afford the product. ¹H NMR (400 MHz, Methanol-d₄) δ 8.32-8.24 (m, 2H), 7.51-7.39 (m, 2H), 7.24-7.12 (m, 2H), 6.97-6.90 (m, 2H), 4.94 (heptd, J=6.2, 3.2 Hz, 1H), 4.12-4.07 (m, 2H), 4.05-3.93 (m, 1H), 3.76-3.68 (m, 2H), 3.41 (d, J=0.5 Hz, 3H), 1.32 (td, J=7.1, 1.2 Hz, 3H), 1.19 (dt, J=6.3, 2.0 Hz, 6H). ³¹P NMR (162 MHz, Methanol-d₄) δ −0.86, −1.06. LCMS: MS m/z=483.06 [M+1], t_{R}=1.39 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.58 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 52. butyl ((4-nitrophenoxy)(phenoxy)phosphoryl)-L-alaninate

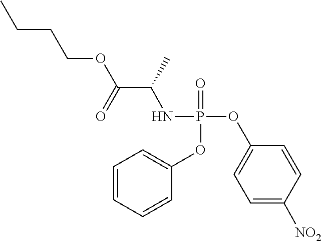 [SMILES: CCCCOC(=O)[C@H](C)NP(=O)(Oc1ccccc1)Oc1ccc([N+](=O)[O-])cc1]

Phenyl dichlorophosphate (0.89 mL, 5.97 mmol) in dichloromethane (12 mL) was added dropwise over 15 minutes to a solution of butyl L-alaninate hydrochloride (CAS #81305-85-3, 1.0 g, 5.97 mmol) in dichloromethane (12 mL) at 0° C. After the addition was complete, triethylamine (2.0 mL, 14.32 mmol) in dichloromethane (2.5 mL) was added over 5 minutes. After 3.5 h, 4-nitrophenol (0.83 g, 5.97 mmol) and triethylamine (1.0 mL, 7.16 mmol) were then sequentially added at 0° C., and the resulting mixture was then allowed to warm to RT. After 2 h, the reaction mixture was diluted with dichloromethane (50 mL), washed with water (2×100 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, methanol-d₄) δ 8.31-8.23 (m, 1H), 7.52-7.34 (m, 2H), 7.32-7.18 (m, 2H), 4.04 (td, J=6.6, 2.7 Hz, 2H), 1.60-1.48 (m, 1H), 1.40-1.26 (m, 3H), 0.89 (t, J=7.4 Hz, 2H). ³¹P NMR (162 MHz, methanol-d₄) δ −1.36, −1.59. LCMS: MS m/z=423.13 [M+1], t_{R}=1.22 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 53. 3-Methoxypropyl ((4-nitrophenoxy)(phenoxy)phosphoryl)-L-alaninate

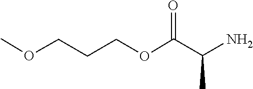

3-Methoxypropyl L-alaninate. To a mixture of Cbz-L-alanine (2.80 g, 12.54 mmol), 3-methoxypropanol (1.00 mL, 10.45 mmol), and EDCI (2.11 g, 13.59 mmol) in acetonitrile (40 mL) was added DMAP (1.92 g, 15.68 mmol). Then the mixture was stirred at room temperature for 15 h, then diluted with EtOAc, washed with brine, dried over sodium sulfate, and concentrated in vacuo. The obtained residue was purified by silica gel chromatography (EtOAc 0 to 50% in hexanes, 35 min run) to give a Cbz-L-alanine ester (2.78 g), which was dissolved in THF (20 mL) and 20% Pd(OH)₂ (800 mg, 1.14 mmol) added at room temperature. The resulting mixture was stirred at room temperature for 4 h under a hydrogen gas atmosphere, filtered, concentrated in vacuo, and dried under high vacuum to afford the product. ¹H NMR (400 MHz, Chloroform-d) δ 4.28-4.14 (m, 2H), 3.55 (q, J=7.0 Hz, 1H), 3.43 (t, J=6.2 Hz, 2H), 3.32 (s, 3H), 1.98-1.85 (m, 4H), 1.33 (d, J=7.0 Hz, 3H). LCMS m/z=161.98 (M+H), t_{R}=0.12 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

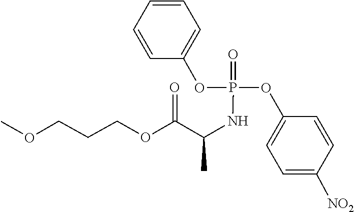 [SMILES: COCCCOC(=O)[C@H](C)NP(=O)(Oc1ccccc1)Oc1ccc([N+](=O)[O-])cc1]

3-Methoxypropyl ((4-nitrophenoxy)(phenoxy)phosphoryl)-L-alaninate. To a solution of 3-Methoxypropyl L-alaninate (1.32 g, 8.20 mmol) in DCM (20 mL) was added phenyl phosphorodichloridate (1.23 mL, 8.20 mmol) in one portion quickly at −78° C. Then triethylamine (1.14 mL, 8.20 mmol) was added over 5 min at −78° C. The resulting mixture was stirred for 30 min after removal of dry ice bath and cooled to −78° C. p-Nitrophenol (1.14 g, 8.20 mmol) was added in one portion and triethylamine (1.14 mL, 8.20 mmol) added over 5 min at −78° C. The resulting mixture was stirred for 2 h after removal of dry ice bath. After dilution with DCM, the mixture was washed with brine, concentrated in vacuo, and the resulting residue purified by silica gel column chromatography (EtOAc 0 to 100% in hexanes) to afford the product. ¹H NMR (400 MHz, Chloroform-d) δ 8.26-8.19 (m, 2H), 7.36 (m, 4H), 7.27-7.15 (m, 3H), 4.20 (m, 2H), 4.17-4.06 (m, 1H), 3.91 (m, 1H), 3.40 (m, 2H), 3.30 (m, 3H), 1.87 (m, 2H), 1.40 (m, 3H). ³¹P NMR (162 MHz, Chloroform-d) δ −3.07, −3.10. LCMS: m/z=439.11 (M+H). t_{R}=1.36 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min

### Intermediate 54. methyl (2S)-2-(((benzyloxy)carbonyl)amino)-3-(4-(((((S)-1-methoxy-1-oxopropan-2-yl)amino)(4-nitrophenoxy)phosphoryl)oxy)phenyl)propanoate

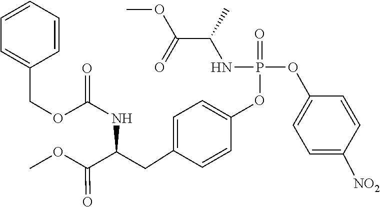 [SMILES: COC(=O)[C@H](C)NP(=O)(Oc1ccc(C[C@H](NC(=O)OCc2ccccc2)C(=O)OC)cc1)Oc1ccc([N+](=O)[O-])cc1]

L-Alanine methyl ester hydrochloride (275 mg, 1.97 mmol) in dichloromethane (20 mL) was added dropwise over 10 minutes to a solution of 4-nitrophenyl phosphorodichloridate (504 mg, 1.97 mmol) in dichloromethane (20 mL) at 0° C. After addition was complete, triethylamine (0.55 mL, 3.93 mmol) was added dropwise. After 60 minutes, N-carbobenzyloxy-L-tyrosine methyl ester (649 mg, 1.97 mmol) and triethylamine (0.28 mL, 1.97 mmol) were sequentially added at 0° C., and the resulting mixture was then allowed to warm to RT. After 3 hr, the reaction mixture was washed with water (2×50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, Methanol-d₄) δ 8.34-8.17 (m, 2H), 7.53-7.37 (m, 2H), 7.37-7.09 (m, 9H), 5.02 (s, 2H), 4.43 (dd, J=9.4, 5.2 Hz, 1H), 4.19-3.97 (m, 1H), 3.70 (s, 3H), 3.62 (d, J=4.4 Hz, 3H), 3.16 (dd, J=14.0, 5.4 Hz, 1H), 2.93 (dd, J=14.1, 9.8 Hz, 1H), 1.32 (td, J=7.3, 1.2 Hz, 3H). ³¹P NMR (162 MHz, Methanol-d₄) δ −1.30, −1.51. LCMS: MS m/z=616.03 [M+1], t_{R}=1.63 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.81 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 55. (S)-Tetrahydrofuran-3-yl ((4-nitrophenoxy)(phenoxy)

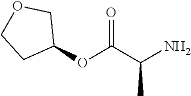

(S)-Tetrahydrofuran-3-yl-L-alaninate. To a mixture of N-Cbz-L-alanine (3.31, 14.83 mmol), (S)-THF-3-ol (1.0 mL, 12.34 mmol), and EDCI (2.49 g, 16.04 mmol) in acetonitrile (20 mL) was added DMAP (2.26 g, 18.51 mmol). Then the mixture was stirred at room temperature for 15 h, then diluted with EtOAc, washed with brine, dried over sodium sulfate, and concentrated in vacuo. The obtained residue was purified by silica gel chromatography (EtOAc 0 to 80% in hexanes) to give a Cbz-L-alanine 4-THF ester, which was dissolved in THF (20 mL) and 20% palladium hydroxide (433 mg, 0.617 mmol) was added at room temperature. The resulting mixture was stirred at room temperature for 2 h under H₂ gas, filtered, and concentrated in vacuo, co-evaporated with DCM multiple times, and dried 15 h under high vacuum to afford the product. ¹H NMR (400 MHz, Chloroform-d) δ 5.37-5.29 (m, 1H), 3.97-3.77 (m, 4H), 3.61-3.52 (m, 1H), 2.27-2.12 (m, 1H), 2.02 (dt, J=12.8, 5.6 Hz, 1H), 1.76 (s, 2H), 1.34 (dd, J=7.1, 1.5 Hz, 3H). LCMS m/z=159.94 (M+H), t_{R}=0.12 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

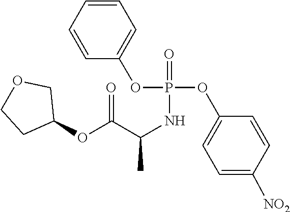 [SMILES: C[C@H](NP(=O)(Oc1ccccc1)Oc1ccc([N+](=O)[O-])cc1)C(=O)O[C@H]1CCOC1]

(S)-Tetrahydrofuran-3-yl ((4-nitrophenoxy)(phenoxy)phosphoryl)-L-alaninate. To a solution of (S)-Tetrahydrofuran-3-yl-L-alaninate (1.45 g, 9.10 mmol) in DCM (20 mL) was added phenyl phosphorodichloridate (1.37 mL, 9.10 mmol) in one portion quickly at −78° C. Then triethylamine (1.27 mL, 9.10 mmol) was added over 5 min at −78° C. The resulting mixture was stirred for 30 min after removal of dry ice bath and cooled to −78° C. p-Nitrophenol (1.27 g, 9.10 mmol) was added in one portion and triethylamine (1.27 mL, 9.10 mmol) added over 5 min at −78° C. The resulting mixture was stirred for 2 h after removal of dry ice bath. After dilution with DCM, the mixture was washed with brine, concentrated in vacuo, and the resulting residue purified by silica gel column chromatography (EtOAc 0 to 100% in hexanes) to afford the product. ¹H NMR (400 MHz, Chloroform-d) δ 8.22 (m, 2H), 7.49-7.31 (m, 4H), 7.30-7.12 (m, 3H), 5.29 (m, 1H), 4.14 (m, 1H), 4.00-3.79 (m, 4H), 3.82-3.60 (m, 1H), 2.17 (m, 1H), 1.95 (m, 1H), 1.40 (m, 3H). ³¹P NMR (162 MHz, Chloroform-d) δ −3.18, −3.20. LCMS: m/z=437.05 (M+H), t_{R}=1.41 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 56. 3-morpholinopropyl ((4-nitrophenoxy)(phenoxy)phosphoryl)-L-alaninate

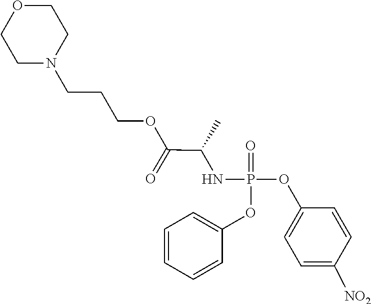 [SMILES: C[C@H](NP(=O)(Oc1ccccc1)Oc1ccc([N+](=O)[O-])cc1)C(=O)OCCCN1CCOCC1]

4-Nitrophenyl phosphorodichloridate (503 mg, 1.97 mmol) in dichloromethane (20 mL) was added dropwise over 10 minutes to a solution of 3-morpholinopropyl L-alaninate hydrochloride (496 mg, 1.97 mmol) in dichloromethane (20 mL) at 0° C. After addition was complete, triethylamine (0.55 mL, 3.93 mmol) was added dropwise. After 90 minutes, phenol (185 mg, 1.97 mmol) and triethylamine (0.28 mL, 1.97 mmol) were sequentially added at 0° C., and the resulting mixture was then allowed to warm to RT. After 30 minutes, the reaction mixture was washed with water (2×50 mL) and brine (50 mL), dried over anhydrous sodium sulfate, and concentrated under reduced pressure. The crude residue was purified by silica gel chromatography eluting with 20-100% ethyl acetate in hexanes to afford the product. ¹H NMR (400 MHz, Methanol-d₄) δ 8.32-8.24 (m, 2H), 7.51-7.39 (m, 2H), 7.24-7.12 (m, 2H), 6.97-6.90 (m, 2H), 4.94 (m, 1H), 4.12-4.07 (m, 2H), 4.05-3.93 (m, 1H), 3.76-3.68 (m, 2H), 3.41 (d, J=0.5 Hz, 3H), 1.32 (td, J=7.1, 1.2 Hz, 3H), 1.19 (dt, J=6.3, 2.0 Hz, 6H). ³¹P NMR (162 MHz, Acetonitrile-d₃) δ −2.12, −2.22. LCMS: MS m/z=494.35 [M+1], t_{R}=1.03 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 57. (R)-Tetrahydrofuran-3-yl ((4-nitrophenoxy)(phenoxy)

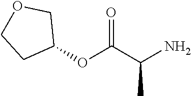

(R)-Tetrahydrofuran-3-yl-L-alaninate. To a mixture of N-Cbz-L-alanine (3.31 g, 14.83 mmol), (R)-THF-3-ol (1.0 mL, 12.34 mmol), and EDCI (2.49 g, 16.04 mmol) in acetonitrile (20 mL) was added DMAP (2.26 g, 18.51 mmol). Then the mixture was stirred at room temperature for 15 h, then diluted with EtOAc, washed with brine, dried over sodium sulfate, and concentrated in vacuo. The obtained residue was purified by silica gel chromatography (EtOAc 0 to 50% in hexanes, 35 min run) to give a Cbz-L-alanine ester (2.78 g), which was dissolved in THF (20 mL) and 20% Pd(OH)₂ (433 mg, 0.617 mmol) added at room temperature. The resulting mixture was stirred at room temperature for 4.5 h under a hydrogen atmosphere, filtered, concentrated in vacuo, and dried under high vacuum to afford the product. ¹H NMR (400 MHz, Chloroform-d) δ 5.32 (ddt, J=6.5, 4.3, 1.9 Hz, 1H), 3.98-3.78 (m, 4H), 3.56 (q, J=7.0 Hz, 1H), 2.19 (dtd, J=13.7, 8.4, 6.4 Hz, 1H), 2.05-1.92 (m, 1H), 1.79 (s, 2H), 1.34 (d, J=7.0 Hz, 3H). LCMS: m/z=159.92 (M+H), t_{R}=0.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

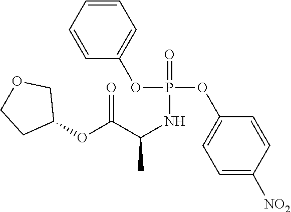 [SMILES: C[C@H](NP(=O)(Oc1ccccc1)Oc1ccc([N+](=O)[O-])cc1)C(=O)O[C@@H]1CCOC1]

(R)-Tetrahydrofuran-3-yl ((4-nitrophenoxy)(phenoxy)phosphoryl)-L-alaninate. To a solution of (R)-Tetrahydrofuran-3-yl-L-alaninate (1.66 g, 10.44 mmol) in DCM (40 mL) was added phenyl phosphorodichloridate (1.56 mL, 10.44 mmol) added in one portion quickly at −78° C. Then triethylamine (1.45 mL, 10.44 mmol) was added over 5 min at −78° C. The resulting mixture was stirred for 30 min after removal of dry ice bath and cooled to −78° C. p-Nitrophenol (1.45 g, 10.44 mmol) was added in one portion and triethylamine (1.45 mL, 10.44 mmol) added over 5 min at −78° C. The resulting mixture was stirred for 2 h after removal of dry ice bath. After dilution with DCM, the mixture was washed with brine, concentrated in vacuo, and the resulting residue purified by silica gel column chromatography (EtOAc 0 to 100% in hexanes) to afford the product. ¹H NMR (400 MHz, Chloroform-d) δ 8.22 (m, 2H), 7.43-7.31 (m, 4H), 7.25-7.14 (m, 3H), 5.29 (m, 1H), 4.21-4.10 (m, 1H), 3.93-3.79 (m, 4H), 3.79-3.71 (m, 1H), 2.17 (m, 1H), 1.97-1.85 (m, 1H), 1.44-1.37 (m, 3H). ³¹P NMR (162 MHz, Chloroform-d) δ −3.24, −3.26. LCMS: m/z=437.02 (M+H), t_{R}=1.42 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Intermediate 58. methyl (chloro(phenoxy)phosphorothioyl)-L-alaninate

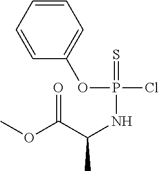 [SMILES: COC(=O)[C@H](C)NP(=S)(Cl)Oc1ccccc1]

Thiophosphoryl chloride (5.08 mL, 50.0 mmol) and triethylamine (6.97 mL, 50.0 mmol) were sequentially added to a solution of phenol (4.70 mg, 50.0 mmol) in TBME (72 mL) at −78° C. under an argon atmosphere. The reaction mixture was then allowed to warm to RT. After 1 h, the resulting mixture was filtered and the filtrate was concentrated under reduced pressure. The residue was dissolved in dichloromethane (72 mL) and L-alanine methyl ester hydrochloride (6.97 mg, 50.0 mmol) was added. The resulting suspension was cooled to −78° C. and triethylamine (13.9 mL, 100 mmol) was added dropwise. The reaction mixture was then allowed to warm to RT. After 16 h, the reaction mixture was concentrated under reduced pressure and TBME (100 mL) was added to the residue. The resulting white solids were removed by vacuum filtration and the filtrate was concentrated under reduced pressure to afford the product used directly in the next step. ¹H NMR (400 MHz, chloroform-d₁) δ 7.45-7.12 (m, 5H), 4.67-4.44 (m, 1H), 4.44-4.24 (m, 1H), 3.81 (s, 1.5H), 3.78 (s, 1.5H), 1.53 (app t, J=6.8 Hz, 3H). ³¹P NMR (162 MHz, chloroform-d₁) δ 64.78 (s), 64.63 (s).

### Intermediate 59. Cyclohexyl ((((S)-1-(2-ethylbutoxy)-1-oxopropan-2-yl)amino)(4-nitrophenoxy)phosphoryl)-L-alaninate and cyclohexyl ((((S)-1-cyclohexyloxy-1-oxopropan-2-yl)amino)(4-nitrophenoxy)phosphoryl)-L-alaninate

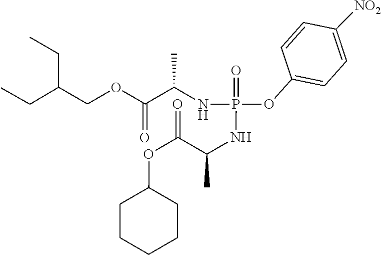 [SMILES: CCC(CC)COC(=O)[C@H](C)NP(=O)(N[C@@H](C)C(=O)OC1CCCCC1)Oc1ccc([N+](=O)[O-])cc1]

To a solution of (S)-1-(cyclohexyloxy)-1-oxopropan-2-aminium chloride Intermediate 11 (680 mg, 3.27 mmol) in THF (10 mL) was added 4-nitrophenyl phosphorodichloridate (838 mg, 3.27 mmol) in one portion. The resulting mixture was cooled in ice bath and triethylamine (1.0 mL, 6.54 mmol) in THF (2 mL) was added over 30 min. The resulting mixture was stirred under ice bath for 1.5 h and (S)-1-(2-ethylbutoxy)-1-oxopropan-2-aminium chloride (687 mg, 3.27 mmol) was added in one portion and triethylamine (1.0 mL, 6.54 mmol) in THF (2 mL) added over 30 min under ice bath. The resulting mixture was stirred under ice bath for 1.5 h, diluted with EtOAc, washed with water and brine, concentrated in vacuo, and the resulting residue purified by preparative HPLC (Phenomenex Gemini-NX 10μ C18 110° A 250×30 mm column, 0%-100% acetonitrile/water gradient in 25 min run) to afford the product. ¹H NMR (400 MHz, Chloroform-d) δ 8.20 (m, 2H), 7.38 (m, 2H), 4.77 (m, 1H), 4.15-3.91 (m, 4H), 3.60 (m, 2H), 1.91-1.77 (m, 2H), 1.75-1.67 (m, 2H), 1.51 (m, 2H), 1.45-1.23 (m, 15H), 0.88 (m, 6H). ³¹P NMR (162 MHz, Chloroform-d) δ 8.04. LCMS: MS m/z=528.10 [M+1].

### Intermediate 60. 4-nitrophenyl-N,N′-cyclohexyl L-alaninatephosphorodiamidate

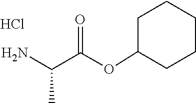

(S)-cyclohexyl 2-aminopropanoate hydrochloride. L-Alanine (891 mg, 10 mmol) was mixed with cyclohexanol (10 mL). Trimethylsilyl chloride (12.7 mL, 100 mmol) was added dropwise and stirred for 20 mins. Reaction mixture was heated to 60° C. and stirred for 16 hrs. Reaction was concentrated under reduced pressure and azeotroped with toluene (5×) to give an oil. Hexanes (100 mL) was added and stirred for 15 hrs to give a solid which was collected, washed with hexanes (100 mL) and dried under high vacuum to give the product. ¹H NMR (400 MHz, DMSO-d₆) δ 8.45 (s, 3H), 4.77 (tt, J=8.4, 3.7 Hz, 1H), 4.02 (q, J=7.2 Hz, 1H), 1.71 (m, 4H), 1.53-1.17 (m, 9H).

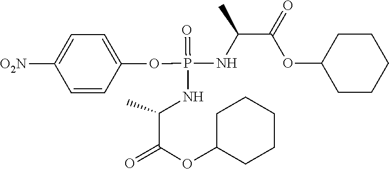 [SMILES: C[C@H](NP(=O)(N[C@@H](C)C(=O)OC1CCCCC1)Oc1ccc([N+](=O)[O-])cc1)C(=O)OC1CCCCC1]

4-nitrophenyl-N,N′-cyclohexyl L-alaninatephosphorodiamidate. 4-Nitrophenyl dichlorophosphate (256 mg, 1 mmol) was dissolved in anhydrous dichloromethane (10 mL) and stirred under atmosphere nitrogen in an ice bath. (S)-cyclohexyl 2-aminopropanoate hydrochloride (415 mg, 2 mmol) was added in one portion. Triethylamine (698 μL, 5 mmol) was added dropwise and stirred for 2 hrs. Reaction was diluted with dichloromethane (15 mL) and washed with 2% aqueous citric acid solution (20 mL). Organic was dried over anhydrous sodium sulfate and concentrated under reduced pressure. The crude was purified via SiO₂ column chromatography (12 g SiO₂ Combiflash HP Gold Column 0-50% ethyl acetate/hexanes) to afford the product. ¹H NMR (400 MHz, DMSO-d₆) δ 8.30-8.13 (m, 2H), 7.49-7.27 (m, 2H), 5.50 (m, 2H), 4.62 (m, 2H), 3.85 (m, 2H), 1.67 (m, 8H), 1.51-1.18 (m, 18H). ³¹P NMR (162 MHz, DMSO-d₆) δ 9.50. MS m/z=526.0 [M+1], 524.1 [M−1].

### Intermediate 61. 4-nitrophenyl-N,N′-isopropyl L-alaninatephosphorodiamidate

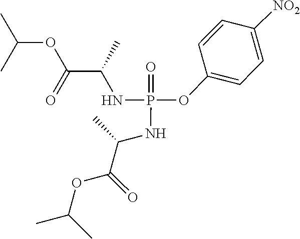 [SMILES: CC(C)OC(=O)[C@H](C)NP(=O)(N[C@@H](C)C(=O)OC(C)C)Oc1ccc([N+](=O)[O-])cc1]

To a solution of isopropyl L-alaninate HCl salt (1.97 g, 11.72 mmol) in DCM (20 mL) was added 4-nitrophenyl phosphorodichloridate (1.5 g, 5.86 mmol) in one portion. The resulting mixture was cooled to about 0° C. and triethylamine (2.37 g, 23.44 mmol) was added dropwise. The resulting mixture was stirred for about 30 min after removal of ice bath and was stirred overnight. The reaction mixture was then diluted with EtOAc, washed with water and brine, the organic solvent was concentrated in vacuum, and the resulting residue was purified by silica gel column chromatography eluting with 0-100% ethyl acetate in hexanes to afford the product. LCMS: MS m/z=445.96 [M+1].

### Intermediate 62. 4-nitrophenyl-N,N′-cyclobutylmethyl L-alaninatephosphorodiamidate

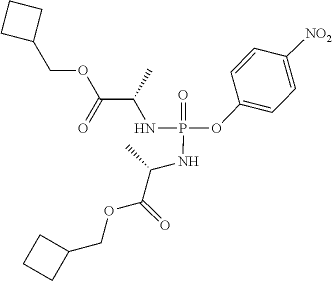 [SMILES: C[C@H](NP(=O)(N[C@@H](C)C(=O)OCC1CCC1)Oc1ccc([N+](=O)[O-])cc1)C(=O)OCC1CCC1]

To a solution of cyclobutylmethyl L-alaninate HCl salt (1.51 g, 7.8 mmol) in DCM (20 mL) was added 4-nitrophenyl phosphorodichloridate (1 g, 3.9 mmol) in one portion. The resulting mixture was cooled to 0° C. and triethylamine (1.58 g, 15.6 mmol) was added dropwise. The resulting mixture was stirred for 30 min after removal of ice bath and was stirred for overnight. The reaction mixture was then diluted with EtOAc, washed with water and brine, the organic solvent was concentrated in vacuum, and the resulting residue was purified by silica gel column chromatography eluting with 0-100% ethyl acetate in hexanes to afford the product. LCMS: MS m/z=497.98 [M+1].

### Intermediate 63. (1r,4S)-4-((tert-butoxycarbonyl)amino)cyclohexyl ((4-nitrophenoxy)(phenoxy)phosphoryl)-L-alaninate

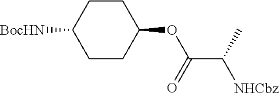 [SMILES: C[C@H](NC(=O)OCc1ccccc1)C(=O)O[C@H]1CC[C@H](NC(=O)OC(C)(C)C)CC1]

(1r,4S)-4-((tert-butoxycarbonyl)amino)cyclohexyl ((benzyloxy)carbonyl)-L-alaninate. 4-Dimethylaminopyridine (2.84 g, 23 mmol) was added to a solution of tert-butyl ((1r,4r)-4-hydroxycyclohexyl)carbamate (4.00 g, 19.0 mmol) and ((benzyloxy)carbonyl)-L-alanine (4.98 g, 22.0 mmol), and EDCI (3.13 g, 20.0 mmol) in acetonitrile (100 mL) at RT. After 4 h, the reaction mixture was diluted with dichloromethane (200 mL), washed with brine (50 mL), dried over anhydrous sodium sulfate, and concentrated under reduced pressure. The crude residue was purified by silica gel chromatography eluting with 0-50% ethyl acetate in hexanes to afford the product. ¹H NMR (400 MHz, CDCl₃) δ 7.40-7.28 (m, 5H), 5.29 (br d, J=7.7 Hz, 1H), 5.10 (s, 2H), 4.78-4.60 (m, 1H), 4.47-4.19 (m, 2H), 3.45 (s, 1H), 2.08-1.89 (m, 4H), 1.54-1.34 (m, 14H), 1.28-1.16 (m, 2H). LCMS: MS m/z=420.99 [M+1].

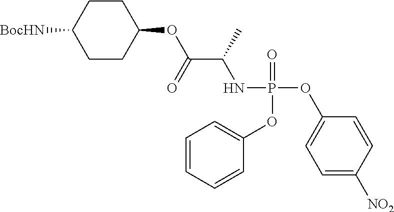 [SMILES: C[C@H](NP(=O)(Oc1ccccc1)Oc1ccc([N+](=O)[O-])cc1)C(=O)O[C@H]1CC[C@H](NC(=O)OC(C)(C)C)CC1]

(1r,4S)-4-((tert-butoxycarbonyl)amino)cyclohexyl ((4-nitrophenoxy)(phenoxy)phosphoryl)-L-alaninate. A hydrogen balloon was appended to a flask containing a solution of (1r,4S)-4-((tert-butoxycarbonyl)amino)cyclohexyl ((benzyloxy)carbonyl)-L-alaninate (1.96 g, 4.66 mmol) and palladium on carbon (10% wt, 2.0 g) in tetrahydrofuran (50 mL) at RT under an argon atmosphere. The vessel was evacuated and refilled with hydrogen atmosphere (3×) and the reaction mixture was stirred vigorously. After 1.5 h, the reaction mixture was filtered through a pad of celite and the filtrate was concentrated under reduced pressure to afford the crude Cbz-deprotected material. The crude residue was taken up into dichloromethane (23 mL) and the resulting mixture was cooled to 0° C. Phenyl dichlorophosphate (0.70 mL, 4.7 mmol) and triethylamine (0.66 mL, 4.7 mmol) were sequentially added. After 1 h, 4-nitrophenol (660 mg, 4.74 mmol) and triethylamine (0.66 mL, 4.7 mmol) were then added. After 1.5 h, the reaction mixture was diluted with dichloromethane (50 mL), washed with saturated aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, chloroform-d₁) δ 8.26-8.18 (m, 2H), 7.43-7.30 (m, 4H), 7.25-7.17 (m, 3H), 4.77-4.58 (m, 1H), 4.40 (br s, 1H), 4.18-3.99 (m, 1H), 3.93-3.80 (m, 1H), 3.44 (br s, 1H), 2.07-1.87 (m, 4H), 1.52-1.36 (m, 14H), 1.30-1.16 (m, 2H). ³¹P NMR (162 MHz, chloroform-d₁) δ −3.15 (s). LCMS: MS m/z=563.88 [M+1].

### Intermediate 64. ((1r,4S)-4-((tert-butoxycarbonyl)amino)cyclohexyl)methyl ((4-nitrophenoxy)(phenoxy)phosphoryl)-L-alaninate

### Method 1.

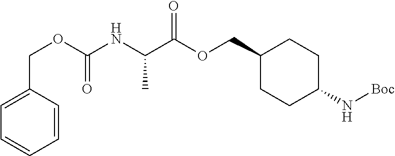 [SMILES: C[C@H](NC(=O)OCc1ccccc1)C(=O)OC[C@H]1CC[C@H](NC(=O)OC(C)(C)C)CC1]

((1r,4S)-4-((tert-butoxycarbonyl)amino)cyclohexyl)methyl ((benzyloxy) carbonyl)-L-alaninate. Cbz-L-Alanine (223 mg, 1.00 mmol) was dissolved in anhydrous MeCN (10 mL). trans-1-(Boc-amino)-4-(hydroxymethyl)cyclohexane (229 mg, 1.00 mmol) and EDCI (230 mg, 1.2 mmol) were added to the reaction, which was then stirred for 25 min. DMAP (122 mg, 1 mmol) was added in one portion, and the reaction was stirred for 4 h. The reaction mixture was diluted with ethyl acetate (15 mL) and washed with 5% aqueous citric acid solution (2×5 mL), followed with brine (10 mL). Organic extract was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions containing the desired product were combined and concentrated under reduced pressure to give the product. ¹H NMR (400 MHz, chloroform-d) δ 7.41-7.27 (m, 5H), 5.29 (d, J=7.6 Hz, 1H), 5.11 (s, 2H), 4.47-4.24 (m, 2H), 3.96 (d, J=6.6 Hz, 2H), 3.37 (bs, 1H), 2.03 (m, 2H), 1.78 (m, 2H), 1.58 (m, 2H), 1.44 (m, 12H), 1.10 (m, 4H).

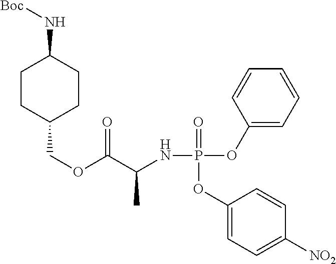 [SMILES: C[C@H](NP(=O)(Oc1ccccc1)Oc1ccc([N+](=O)[O-])cc1)C(=O)OC[C@H]1CC[C@H](NC(=O)OC(C)(C)C)CC1]

((1r,4S)-4-((tert-butoxycarbonyl)amino)cyclohexyl)methyl ((4-nitrophenoxy)(phenoxy)phosphoryl)-L-alaninate. ((1r,4S)-4-((tert-butoxycarbonyl)amino)cyclohexyl)methyl ((benzyloxy) carbonyl)-L-alaninate (348 mg, 0.800 mmol) was dissolved in 12 mL of anhydrous tetrahydrofuran. Degussa type 10% Palladium on carbon (25 mg) was added to the reaction and then stirred under atmospheric hydrogen for 3 h. Palladium on carbon was filtered off, and the filtrate was used in the next reaction without further purification. Phenyl dichlorophosphate (119 μL, 0.800 mmol) was dissolved in 15 mL anhydrous dichloromethane and stirred under atmospheric nitrogen in an ice bath. The filtrate from above was then added to the reaction solution dropwise and then stirred for 30 min. Triethylamine (120 μL, 0.88 mmol) was added dropwise and stirred for 1 h. p-Nitrophenol (100 mg, 0.72 mmol) was added in one portion. Triethylamine (123 μL, 0.88 mol) was added dropwise, and the reaction mixture was stirred for 2 h at RT. The reaction mixture was then diluted with dichloromethane (10 mL) and washed with water (3×10 mL). The organic extract was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions containing the desired product were combined and concentrated under reduced pressure to give the product. ¹H NMR (400 MHz, chloroform-d) δ 8.27-8.18 (m, 2H), 7.44-7.30 (m, 4H), 7.27-7.17 (m, 3H), 4.35 (s, 1H), 4.22-4.06 (m, 1H), 3.99-3.88 (m, 2H), 3.85 (t, J=10.6 Hz, 1H), 3.36 (s, 1H), 2.03 (m, 2H), 1.75 (m, 2H), 1.57 (m, 2H), 1.48-1.36 (m, 12H), 1.15-0.98 (m, 4H). ³¹P NMR (162 MHz, chloroform-d) δ 3.12, 3.13. LCMS: MS m/z=478.2 [M+1].

### Method 2.

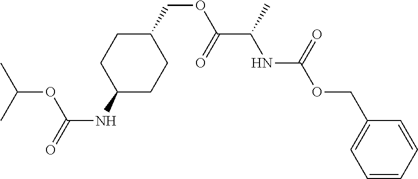 [SMILES: CC(C)OC(=O)N[C@H]1CC[C@H](COC(=O)[C@H](C)NC(=O)OCc2ccccc2)CC1]

((1r,4S)-4-((tert-butoxycarbonyl)amino)cyclohexyl)methyl ((benzyloxy)carbonyl)-L-alaninate. trans-1-((tert-Butoxycarbonyl)amino)-4-(hydroxymethyl)cyclohexane (510 mg, 2.18 mmol) followed by N-(3-dimethylaminopropyl)-N′-ethylcarbodiimide hydrochloride (509 g, 2.62 mmol) were added to a solution of Z-Ala-OH (489 g, 2.18 mmol) in acetonitrile (22 mL) at RT. After 30 min, 4-(dimethylamino)pyridine (267 mg, 2.18 mmol) was added. After 18 h, the reaction was diluted with ethyl acetate (100 mL) and the resulting mixture was washed with 10% aqueous citric acid (2×100 mL) and brine (100 mL). The organic layer was dried over anhydrous sodium sulfate and concentrated under reduced pressure. The crude residue was subjected to silica gel chromatography eluting with 0-50% methanol in ethyl acetate to afford the product. ¹H NMR (400 MHz, Chloroform-d) δ 7.41-7.29 (m, 5H), 5.28 (s, 1H), 5.11 (s, 2H), 4.46-4.27 (m, 2H), 3.96 (d, J=6.6 Hz, 2H), 3.37 (s, 1H), 2.03 (s, 2H), 1.78 (s, 2H), 1.56 (s, 2H), 1.44 (s, 9H), 1.42 (d, J=7.2 Hz, 3H), 1.08 (t, J=9.7 Hz, 4H). LCMS: MS m/z=434.87 [M+1], t_{R}=1.21 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.96 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

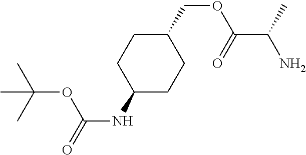 [SMILES: C[C@H](N)C(=O)OC[C@H]1CC[C@H](NC(=O)OC(C)(C)C)CC1]

((1r,4S)-4-((tert-butoxycarbonyl)amino)cyclohexyl)methyl L-alaninate. Palladium on carbon (198 mg, 10 wt %) was added to a solution of ((1r,4S)-4-((tert-butoxycarbonyl)amino)cyclohexyl)methyl ((benzyloxy)carbonyl)-L-alaninate (719 g, 1.65 mmol) in tetrahydrofuran (24 mL) that was purged with argon. The mixture was then purged with hydrogen and stirred at RT. After 1 h, the mixture was filtered through celite, the filter was rinsed with tetrahydrofuran, and the volatiles were removed under reduce pressure to obtain the product. ¹H NMR (400 MHz, Chloroform-d) δ 4.38 (s, 1H), 4.02-3.85 (m, 2H), 3.55 (q, J=7.0 Hz, 1H), 3.38 (s, 1H), 2.04 (d, J=7.1 Hz, 2H), 1.83-1.73 (m, 2H), 1.63 (s, 2H), 1.44 (s, 10H), 1.34 (d, J=7.0 Hz, 3H), 1.09 (t, J=10.0 Hz, 4H). LCMS: MS m/z=300.93 [M+1], t_{R}=0.65 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

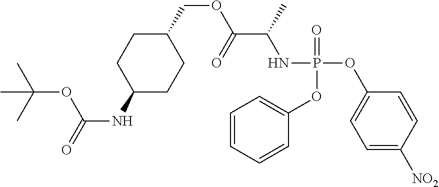 [SMILES: C[C@H](NP(=O)(Oc1ccccc1)Oc1ccc([N+](=O)[O-])cc1)C(=O)OC[C@H]1CC[C@H](NC(=O)OC(C)(C)C)CC1]

((1r,4S)-4-((tert-butoxycarbonyl)amino)cyclohexyl)methyl ((4-nitrophenoxy)(phenoxy)phosphoryl)-L-alaninate. To ((1r,4S)-4-((tert-butoxycarbonyl)amino)cyclohexyl)methyl L-alaninate (553 mg, 1.65 mmol) in tetrahydrofuran (24 mL) at 0° C. was added a solution of phenyl dichlorophosphate (247 μL, 1.65 mmol) in dichloromethane (30 mL) slowly over 15 min. After the addition was complete, triethylamine (0.26 mL, 1.82 mmol) was added dropwise. After 1 h, 4-nitrophenol (240 mg, 1.65 mmol) and triethylamine (0.26 mL, 1.82 mmol) were then sequentially added at 0° C., and the resulting mixture was then allowed to warm to RT. After 1 h, the reaction mixture was diluted with dichloromethane (50 mL) and washed with water (3×75 mL). The organic layer was dried over anhydrous sodium sulfate and concentrated under reduced pressure. The crude residue was purified by silica gel chromatography eluting with 0-100% ethyl acetate in hexanes to afford the product. ¹H NMR (400 MHz, Chloroform-d) δ 8.23 (ddd, J=9.3, 1.3, 0.6 Hz, 2H), 7.44-7.31 (m, 4H), 7.25-7.16 (m, 3H), 4.36 (s, 1H), 4.22-4.06 (m, 1H), 3.96-3.90 (m, 2H), 3.84 (t, J=10.6 Hz, 1H), 3.36 (s, 1H), 2.02 (s, 2H), 1.83-1.68 (m, 2H), 1.57 (s, 2H), 1.44 (s, 9H), 1.41 (dd, J=7.1, 3.2 Hz, 3H), 1.06 (t, J=9.6 Hz, 3H). ³¹P NMR (162 MHz, Chloroform-d) δ −3.13 (d, J=2.9 Hz). LCMS: MS m/z=577.8 [M+1], t_{R}=1.28 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=6.35 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 65. 4-nitrophenyl-N,N′-butryl L-alaninatephosphorodiamidate

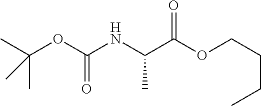 [SMILES: CCCCOC(=O)[C@H](C)NC(=O)OC(C)(C)C]

butyl (tert-butoxycarbonyl)-L-alaninate. Boc-L-alanine (380 mg, 2.0 mmol) was dissolved in anhydrous MeCN (10 mL). 1-Butanol (920 μL, 10.0 mmol) and EDCI (460 mg, 2.4 mmol) were added to the reaction which was then stirred for 15 min. DMAP (240 mg, 2.0 mmol) was added in one portion, and the reaction was stirred for 14 h. The reaction mixture was diluted reaction with ethyl acetate (15 mL) and washed with saturated aqueous sodium bicarbonate solution (2×10 mL), followed with brine (5 mL). The organic extracts were dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions containing the desired product were combined and concentrated under reduced pressure to give the product. ¹H NMR (400 MHz, chloroform-d) δ 5.04 (m, 1H), 4.29 (m, 1H), 4.18-4.07 (m, 2H), 1.67-1.59 (m, 2H), 1.44 (s, 9H), 1.38 (m, 5H), 0.93 (t, J=7.4 Hz, 3H).

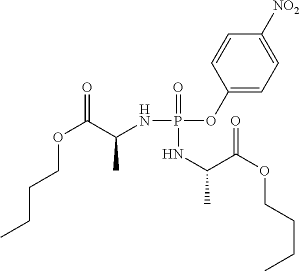 [SMILES: CCCCOC(=O)[C@H](C)NP(=O)(N[C@@H](C)C(=O)OCCCC)Oc1ccc([N+](=O)[O-])cc1]

4-nitrophenyl-N,N′-butryl L-alaninatephosphorodiamidate. Butyl (tert-butoxycarbonyl)-L-alaninate (291 mg, 1.18 mmol) was dissolved in 7 mL of 4 M HCl in dioxane and stirred for 1 h. The reaction mixture was concentrated under reduced pressure to give an oil which was then dissolved in anhydrous dichloromethane (10 mL) and stirred under atmospheric nitrogen in an ice bath. 4-Nitrophenyl phosphorodichloridate (152 mg, 0.59 mmol) was added in one portion, and the reaction was stirred for 10 min. Triethylamine (270 μL, 1.95 mmol) was dissolved in 1 mL of anhydrous dichloromethane and added to the reaction solution dropwise. The reaction mixture was stirred for 1 h. Triethylamine (270 μL, 1.95 mmol) was dissolved with 700 μL of anhydrous dichloromethane and added to reaction dropwise. The reaction mixture was stirred for 16 h at RT. The reaction mixture was diluted with dichloromethane (15 mL) and washed with water (3×20 mL). The organic extract was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions containing the desired product were combined and concentrated under reduced pressure to give the product. ¹H NMR (400 MHz, chloroform-d) δ 8.27-8.15 (m, 2H), 7.43-7.34 (m, 2H), 4.19-3.98 (m, 5H), 3.80-3.61 (m, 1H), 3.58 (m, 2H), 1.67-1.59 (m, 4H), 1.45-1.30 (m, 10H), 0.93 (m, 6H). ³¹P NMR (162 MHz, chloroform-d) δ 7.93. LCMS: MS m/z=474.0 [M+1].

### Intermediate 66. methyl (2S)-2-(((benzyloxy)carbonyl)amino)-3-(4-(((((S)-1-isopropoxy-1-oxopropan-2-yl)amino)(4-nitrophenoxy)phosphoryl)oxy)phenyl)propanoate

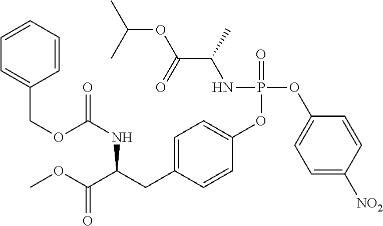 [SMILES: COC(=O)[C@H](Cc1ccc(OP(=O)(N[C@@H](C)C(=O)OC(C)C)Oc2ccc([N+](=O)[O-])cc2)cc1)NC(=O)OCc1ccccc1]

4-Nitrophenyl phosphorodichloridate (504 mg, 1.97 mmol) in dichloromethane (20 mL) was added dropwise over 10 minutes to a solution of L-alanine isopropyl ester hydrochloride (330 mg, 1.97 mmol) in dichloromethane (20 mL) at 0° C. After addition was complete, triethylamine (0.55 mL, 3.93 mmol) was added dropwise. After 60 minutes, N-carbobenzyloxy-L-tyrosine methyl ester (649 mg, 1.97 mmol) and triethylamine (0.28 mL, 1.97 mmol) were sequentially added at 0° C., and the resulting mixture was then allowed to warm to RT. After 30 minutes, the reaction mixture was washed with water (2×50 mL) and brine (50 mL), dried over anhydrous sodium sulfate, and concentrated under reduced pressure. The crude residue was purified by silica gel chromatography eluting with 20-100% ethyl acetate in hexanes to afford the product. ¹H NMR (400 MHz, Methanol-d₄) δ 8.32-8.22 (m, 2H), 7.49-7.37 (m, 2H), 7.35-7.13 (m, 9H), 5.02 (s, 2H), 4.93 (pd, J=6.3, 1.1 Hz, 1H), 4.43 (dd, J=9.4, 5.2 Hz, 1H), 4.00 (dtd, J=10.1, 7.7, 6.5 Hz, 1H), 3.70 (s, 3H), 3.15 (dd, J=14.0, 5.4 Hz, 1H), 2.93 (dd, J=13.9, 9.6 Hz, 1H), 1.32 (td, J=7.2, 1.2 Hz, 3H), 1.20-1.16 (m, 6H). ³¹P NMR (162 MHz, Methanol-d₄) δ −1.26, −1.49. LCMS: MS m/z=644.11 [M+1], t_{R}=1.56 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=6.21 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 67. 2-morpholinoethyl ((4-nitrophenoxy)(phenoxy)phosphoryl)-L-alaninate

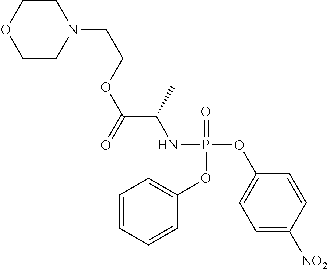 [SMILES: C[C@H](NP(=O)(Oc1ccccc1)Oc1ccc([N+](=O)[O-])cc1)C(=O)OCCN1CCOCC1]

4-Nitrophenyl phosphorodichloridate (505 mg, 1.97 mmol) in dichloromethane (20 mL) was added dropwise over 10 minutes to a solution of 2-morpholinoethyl L-alaninate hydrochloride (496 mg, 1.97 mmol) in dichloromethane (20 mL) at 0° C. After addition was complete, triethylamine (0.55 mL, 3.93 mmol) was added dropwise. After 90 minutes, phenol (185 mg, 1.97 mmol) and triethylamine (0.28 mL, 1.97 mmol) were sequentially added at 0° C., and the resulting mixture was then allowed to warm to RT. After 30 minutes, the reaction mixture was washed with water (2×50 mL) and brine (50 mL), dried over anhydrous sodium sulfate, and concentrated under reduced pressure. The crude residue was purified by silica gel chromatography eluting with 20-100% ethyl acetate in hexanes to afford the product. ¹H NMR (400 MHz, CDCl₃) δ 8.28-8.14 (m, 2H), 7.41-7.29 (m, 4H), 7.24-7.16 (m, 4H), 6.87-6.81 (m, 1H), 4.14-4.04 (bs, 2H), 2.61-2.57 (bs, 4H), 2.45-3.40 (bs, 4H), 1.42 (dt, J=6.3, 2.0 Hz, 6H). ³¹P NMR (162 MHz, CDCl₃) δ −2.70. LCMS: MS m/z=480.27 [M+1], t_{R}=0.96 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.23 min; HPLC system: Agilent 1100 series; Column: Kinetx 2.6 u 100 A C18, 100 mm×4.6 mm; Solvents: Acetonitrile with 0.1% TFA, Water with 0.1% TFA; Gradient: 0 min-8.5 min 2-98% ACN, 8.5 min-10.0 min 98% ACN at 1.5 mL/min.

### Intermediate 68. 2-(diisopropylamino)ethyl ((4-nitrophenoxy)(phenoxy)phosphoryl)-L-alaninate

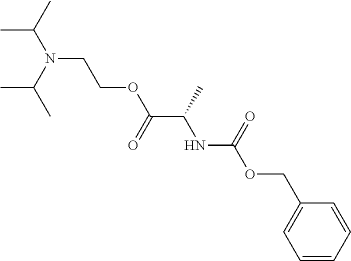 [SMILES: CC(C)N(CCOC(=O)[C@H](C)NC(=O)OCc1ccccc1)C(C)C]

2-(diisopropylamino)ethyl ((benzyloxy)carbonyl)-L-alaninate. N-(3-Dimethylaminopropyl)-N′-ethylcarbodiimide hydrochloride (2.06 g, 10.8 mmol) was added to a solution of Z-Ala-OH (2.00 g, 8.96 mmol) and 2-(diisopropylamino)ethanol (3.2 mL, 17.9 mmol) in acetonitrile (125 mL) at RT. After 10 min, 4-(dimethylamino)pyridine (1.09 g, 8.96 mmol) was added. After 2 d, the reaction mixture was concentrated to half the volume, and the mixture was diluted with ethyl acetate (100 mL) and the resulting mixture was washed with saturated aqueous sodium carbonate solution (100 mL) and brine (100 mL). The organic layer was dried over anhydrous sodium sulfate and concentrated under reduced pressure. The crude residue was subjected to silica gel chromatography eluting with 0-20% methanol in ethyl acetate to afford the product. ¹H NMR (400 MHz, Acetonitrile-d₃) δ 7.48-7.23 (m, 5H), 5.96 (s, 1H), 5.07 (s, 2H), 4.30-4.00 (m, 3H), 2.28 (t, J=7.1 Hz, 2H), 2.14 (s, 6H), 1.73 (p, J=6.9 Hz, 2H), 1.34 (d, J=7.3 Hz, 3H). LCMS: MS m/z=351.26 [M+1], t_{R}=1.05 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.10 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

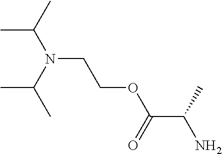

2-(diisopropylamino)ethyl L-alaninate. Palladium on carbon (587 mg, 10 wt %) was added to a solution of 2-(diisopropylamino)ethyl ((benzyloxy)carbonyl)-L-alaninate [symbol] (1.93 g, 5.52 mmol) in ethanol (50 mL) that was purged with argon. The mixture was then purged with hydrogen and stirred at RT. After 18 hr, the mixture was filtered through celite, the filter was rinsed with ethyl acetate, and the volatiles were removed under reduce pressure to afford the product. ¹H NMR (400 MHz, Acetonitrile-d₃) δ 4.06-3.90 (m, 2H), 3.43 (q, J=7.0 Hz, 1H), 3.01 (hept, J=6.5 Hz, 2H), 2.65 (t, J=6.9 Hz, 2H), 1.22 (d, J=7.0 Hz, 3H), 0.99 (d, J=6.6 Hz, 12H). LCMS: MS m/z=217.01 [M+1], t_{R}=0.17 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

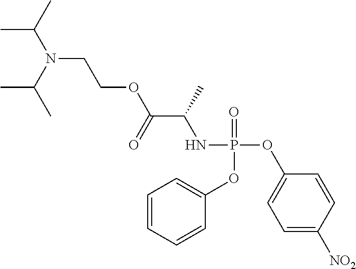 [SMILES: CC(C)N(CCOC(=O)[C@H](C)NP(=O)(Oc1ccccc1)Oc1ccc([N+](=O)[O-])cc1)C(C)C]

2-(diisopropylamino)ethyl ((4-nitrophenoxy)(phenoxy)phosphoryl)-L-alaninate. 2-(diisopropylamino)ethyl L-alaninate (511 mg, 2.43 mmol) in tetrahydrofuran (7 mL) was added dropwise over 15 minutes to a solution of phenyl dichlorophosphate (0.36 mL, 2.43 mmol) in tetrahydrofuran (25 mL) at 0° C. After the addition was complete, triethylamine (0.36 mL, 2.43 mmol) was added dropwise. After 90 min, 4-nitrophenol (337 mg, 2.43 mmol) and triethylamine (1.0 mL, 7.16 mmol) were then sequentially added at 0° C., and the resulting mixture was then allowed to warm to RT. After 17 h, the reaction mixture was diluted with ethyl acetate (100 mL) and washed with water (2×100 mL) and brine (100 mL). The organic layer was dried over anhydrous sodium sulfate and concentrated under reduced pressure to afford the product. ¹H NMR (400 MHz, Acetonitrile-d₃) δ 8.29-8.18 (m, 2H), 7.49-7.35 (m, 4H), 7.30-7.21 (m, 3H), 4.71-4.52 (m, 1H), 4.12-3.99 (m, 2H), 4.00-3.83 (m, 3H), 3.06-2.86 (m, 2H), 2.56 (td, J=7.0, 3.8 Hz, 2H), 1.31 (ddd, J=7.1, 4.7, 1.1 Hz, 4H), 0.94 (d, J=6.5 Hz, 13H). ³¹P NMR (162 MHz, Acetonitrile-d₃) δ −2.15, −2.30. LCMS: MS m/z=494.25 [M+1], t_{R}=1.27 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.97 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 69. isopropyl (2S)-2-(((benzyloxy)carbonyl)amino)-3-(4-(((((S)-1-methoxy-1-oxopropan-2-yl)amino)(4-nitrophenoxy)phosphoryl)oxy)phenyl)propanoate

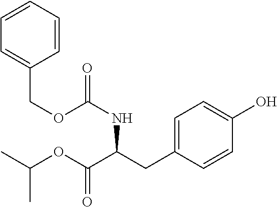 [SMILES: CC(C)OC(=O)[C@H](Cc1ccc(O)cc1)NC(=O)OCc1ccccc1]

isopropyl ((benzyloxy)carbonyl)-L-tyrosinate. Benzyl chloroformate (0.94 mL, 6.58 mmol) was added dropwise to a mixture of L-tyrosine isopropyl ester (1.0 g, 4.48 mmol) in acetone (4.5 mL) and 7 wt % aqueous sodium carbonate (4.5 mL). After 2 hr, reaction mixture was diluted with ethyl acetate (25 mL) and the resulting mixture was washed with water (10 mL) and brine (10 mL). The organic layer was dried over anhydrous sodium sulfate and concentrated under reduced pressure. The crude residue was subjected to silica gel chromatography eluting with 0-100% ethyl acetate in hexanes to afford the product. ¹H NMR (400 MHz, Methanol-d₄) δ 7.39-7.20 (m, 5H), 7.06-6.97 (m, 2H), 6.74-6.62 (m, 2H), 5.05 (d, J=2.6 Hz, 2H), 4.94 (p, J=6.3 Hz, 1H), 4.31 (dd, J=8.6, 6.1 Hz, 1H), 2.99 (dd, J=13.9, 6.1 Hz, 1H), 2.84 (dd, J=13.9, 8.6 Hz, 1H), 1.22 (d, J=6.3 Hz, 3H), 1.14 (d, J=6.3 Hz, 3H). LCMS: MS m/z=357.87 [M+1], t_{R}=1.36 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.19 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

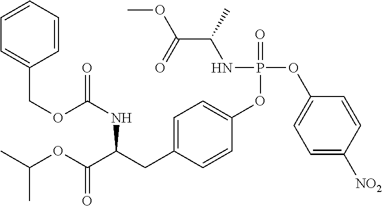 [SMILES: COC(=O)[C@H](C)NP(=O)(Oc1ccc(C[C@H](NC(=O)OCc2ccccc2)C(=O)OC(C)C)cc1)Oc1ccc([N+](=O)[O-])cc1]

isopropyl (2S)-2-(((benzyloxy)carbonyl)amino)-3-(4-(((((S)-1-methoxy-1-oxopropan-2-yl)amino)(4-nitrophenoxy)phosphoryl)oxy)phenyl)propanoate. L-Alanine isopropyl ester hydrochloride (97.2 mg, 0.70 mmol) in dichloromethane (8.0 mL) was added to a solution of 4-nitrophenyl phosphorodichloridate (179.7 mg, 0.70 mmol) in dichloromethane (7.5 mL) at 0° C. After addition was complete, triethylamine (0.22 mL, 1.57 mmol) was added dropwise. After 60 minutes, isopropyl ((benzyloxy)carbonyl)-L-tyrosinate (250.9 mg, 0.70 mmol) in dichloromethane (8.0 mL) and triethylamine (0.11 mL, 0.78 mmol) were sequentially added at 0° C., and the resulting mixture was then allowed to warm to RT. After 20 minutes, the reaction mixture was washed with water (2×20 mL) and brine (20 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, Methanol-d₄) δ 8.32-8.24 (m, 2H), 7.43 (ddd, J=16.0, 9.2, 1.1 Hz, 2H), 7.36-7.09 (m, 9H), 5.03 (s, 2H), 4.97 (p, J=6.2 Hz, 1H), 4.35 (d, J=8.2 Hz, 1H), 4.14-3.95 (m, 1H), 3.62 (d, J=4.5 Hz, 3H), 3.12 (dt, J=12.6, 5.9 Hz, 1H), 2.92 (t, J=11.6 Hz, 1H), 1.35-1.30 (m, 3H), 1.22 (d, J=6.2 Hz, 3H), 1.16 (d, J=6.2 Hz, 4H). ³¹P NMR (162 MHz, Methanol-d₄) δ −1.31, −1.52. LCMS: MS m/z=644.07 [M+1], t_{R}=1.56 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=6.17 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Intermediate 70. isopropyl ((2-(methylthio)ethoxy)(4-nitrophenoxy)phosphoryl)-L-alaninate

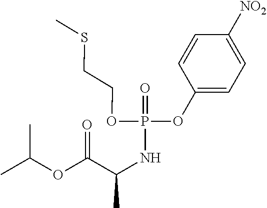 [SMILES: CSCCOP(=O)(N[C@@H](C)C(=O)OC(C)C)Oc1ccc([N+](=O)[O-])cc1]

4-Nitrophenyl phosphorodichloridate (512 mg, 2 mmol) was mixed with 10 mL of anhydrous dichloromethane and stirred under atmospheric nitrogen in an ice bath. L-Alanine isopropyl ester hydrogen chloride (335 mg, 2 mmol) was dissolved in anhydrous dichloromethane (3 mL) and added to the reaction dropwise. The reaction mixture was stirred for 30 min. Triethylamine (927 μL, 6.6 mmol) was dissolved in anhydrous dichloromethane (1 mL) and added to reaction dropwise, and the reaction was stirred for 60 min. 2-(methylthio)ethanol (74 μL, 2 mmol) was added in one portion, and the reaction mixture was stirred for 16 h. The reaction mixture was diluted with dichloromethane (15 mL) and washed with water (3×20 mL). The organic extract was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions containing the desired product were combined and concentrated under reduced pressure to give the product. ¹H NMR (400 MHz, chloroform-d) δ 8.27-8.18 (m, 2H), 7.44-7.33 (m, 2H), 5.02 (m, 1H), 4.33-4.21 (m, 2H), 4.07-3.94 (m, 1H), 3.70 (m, 1H), 2.84-2.73 (m, 2H), 2.14 (m, 3H), 1.40 (m, 3H), 1.29-1.19 (m, 6H). ³¹P NMR (162 MHz, chloroform-d) δ 2.08, 2.20. LCMS: MS m/z=834.5 [2M+Na]; 405.1 [M−1], t_{R}=1.33 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=3.60 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 71. isopropyl ((2-methoxyethoxy)(4-nitrophenoxy)phosphoryl)-L-alaninate

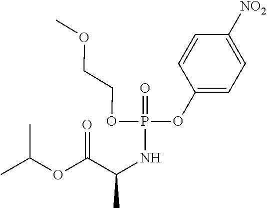 [SMILES: COCCOP(=O)(N[C@@H](C)C(=O)OC(C)C)Oc1ccc([N+](=O)[O-])cc1]

4-Nitrophenyl phosphorodichloridate (512 mg, 2 mmol) was mixed with 10 mL of anhydrous dichloromethane and stirred under atmospheric nitrogen in an ice bath. L-Alanine isopropyl ester hydrogen chloride (335 mg, 2 mmol) was dissolved in anhydrous dichloromethane (3 mL) and added to the reaction dropwise. The reaction mixture was stirred for 30 min. Triethylamine (927 μL, 6.6 mmol) was dissolved in anhydrous dichloromethane (1 mL) and added to the reaction mixture dropwise. The reaction mixture was stirred for 60 min. 2-methoxyethanol (158 μL, 2 mmol) was added in one portion, and the reaction mixture was stirred for 16 h. The reaction mixture was diluted with dichloromethane (15 mL) and washed with water (3×10 mL). The organic extract was dried over anhydrous sodium sulfate and concentrated under reduced pressure. The crude residue was purified via SiO₂ column chromatography (4 g SiO₂ Combiflash HP Gold Column, 0-50% ethyl acetate/hexanes). Fractions containing the desired product were combined and concentrated under reduced pressure to give the product. ¹H NMR (400 MHz, chloroform-d) δ 8.25-8.15 (m, 2H), 7.43-7.32 (m, 2H), 5.00 (m, 1H), 4.36-4.17 (m, 2H), 4.06-3.82 (m, 2H), 3.65-3.55 (m, 2H), 3.37 (m, 3H), 1.41-1.34 (m, 3H), 1.27-1.18 (m, 6H). ³¹P NMR (162 MHz, chloroform-d) δ 2.52, 2.69. LCMS: MS m/z=391.0 [M+1]; 389.1 [M−1], t_{R}=1.24 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.29 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 72. isopropyl ((2-(methylsulfonyl)ethoxy)(4-nitrophenoxy)phosphoryl)-L-alaninate

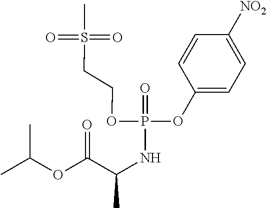 [SMILES: CC(C)OC(=O)[C@H](C)NP(=O)(OCCS(C)(=O)=O)Oc1ccc([N+](=O)[O-])cc1]

Phosphorous oxychloride (280 μL, 3 mmol) was dissolved in anhydrous tetrahydrofuran (10 mL) and stirred in an ice bath under atmospheric nitrogen. 2-(methylsulfonyl)ethanol (280 μL, 3 mmol) was dissolved in anhydrous tetrahydrofuran (2 mL) and added to reaction dropwise. Reaction was stirred for 1 h. L-Alanine isopropyl ester hydrochloride (503 mg, 3 mmol) was added in one portion and the reaction mixture was stirred for 1 h. Triethylamine (1.38 mL, 9.9 mmol) was dissolved in anhydrous tetrahydrofuran (2 mL) and added to the reaction dropwise. The reaction was stirred for 90 min. p-Nitrophenol (417 mg, 3 mmol) was added in one portion. Triethylamine (460 μL, 3.3 mmol) was added. The reaction mixture was stirred for 16 h.

The reaction mixture was then diluted with ethyl acetate (20 mL) and washed with water (5×15 mL) followed with brine (5 mL). The organic extract was dried over anhydrous sodium sulfate and concentrated under reduced pressure. The crude residue was purified via SiO₂ column chromatography (4 g SiO₂ Combiflash HP Gold Column, 0-80% ethyl acetate/hexanes). Fractions containing the desired product were combined and concentrated under reduced pressure to give the product. ¹H NMR (400 MHz, chloroform-d) δ 8.28-8.16 (m, 2H), 7.44-7.32 (m, 2H), 5.00 (m, 1H), 4.71-4.51 (m, 2H), 4.06-3.85 (m, 2H), 3.51-3.33 (m, 2H), 2.96 (m, 3H), 1.40-1.35 (m, 3H), 1.27-1.20 (m, 6H). ³¹P NMR (162 MHz, chloroform-d) δ 2.06, 2.29. LCMS: MS m/z=439.0 [M+1]; t_{R}=1.18 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=3.08 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

### Intermediate 73. 2-(2-ethoxyethoxy)ethyl ((perfluorophenoxy)(phenoxy)phosphoryl)-L-alaninate single isomer

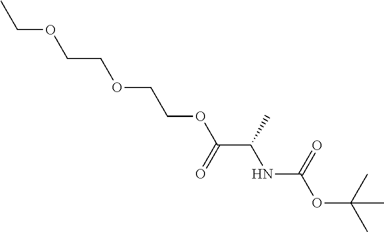 [SMILES: CCOCCOCCOC(=O)[C@H](C)NC(=O)OC(C)(C)C]

2-(2-ethoxyethoxy)ethyl (tert-butoxycarbonyl)-L-alaninate. To a stirred solution of (tert-butoxycarbonyl)-L-alanine (12.41 g, 66 mmol) and 2-(2-ethoxyethoxy)ethan-1-ol (8.00 g, 60 mmol) in dry dichloromethane (100 mL) were added N-methylmorpholine (19.67 mL, 179 mmol), 4-(dimethylamino)pyridine (0.15 g, 1.2 mmol) and tri-propylphosphonic acid cyclic anhydride (42.6 mL, 72 mmol, 50% in ethyl acetate) at 0° C. under an atmosphere of argon. The reaction mixture was then stirred at room temperature for 2 hours. The reaction mixture was washed with water (50 mL), twice with 10% solution of citric acid (2×40 mL), twice with saturated aqueous sodium bicarbonate solution (2×40 mL) and once with brine (50 mL), dried over sodium sulfate, filtered through a 3 cm layer of silica gel which was washed with additional dichloromethane. The combined organics were concentrated down under reduced pressure, co-distilled with dichloromethane and dried under high vacuum overnight to afford the product. ¹H NMR (400 MHz, DMSO-d₆) δ 7.27 (d, J=7.4 Hz, 1H), 4.23-4.14 (m, 1H), 4.14-4.06 (m, 1H), 4.05-3.94 (m, 1H), 3.64-3.56 (m, 2H), 3.55-3.49 (m, 2H), 3.49-3.39 (m, 4H), 1.38 (s, 9H), 1.23 (d, J=7.4 Hz, 3H), 1.09 (t, J=7.0 Hz, 3H).

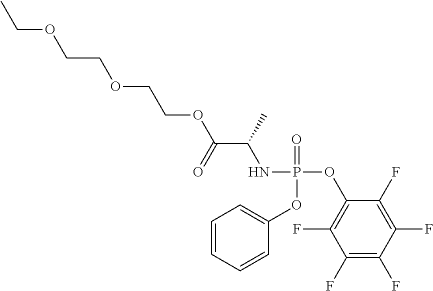 [SMILES: CCOCCOCCOC(=O)[C@H](C)NP(=O)(Oc1ccccc1)Oc1c(F)c(F)c(F)c(F)c1F]

2-(2-ethoxyethoxy)ethyl ((perfluorophenoxy)(phenoxy)phosphoryl)-L-alaninate single isomer. The intermediate 2-(2-ethoxyethoxy)ethyl (tert-butoxycarbonyl)-L-alaninate (18.3 g, 59.93 mmol) was dissolved in 50 mL of 4 M HCl in 1,4-dioxane and the reaction mixture was stirred at room temperature for 2 hours, concentrated under reduced pressure and co-distilled with toluene to give the solid which was dried under high vacuum for 1 hour. The solids were suspended in dichloromethane (100 mL) and phenyl dichlorophosphate (9.81 mL, 65.92 mmol) and triethylamine (18.28 mL, 131.84 mmol) were sequentially added at −78° C. and the resulting mixture was stirred at room temperature for 2 hours. The reaction mixture was cooled down to 0° C. and pentafluorophenol (11.03 g, 59.93 mmol) and triethylamine (10.80 mL, 78.05 mmol) were then sequentially added and the resulting mixture was then allowed to warm to room temperature. After 3 hours, the reaction mixture was cooled down to 0° C. and solids were filtered off, the filtrate was washed with saturated ammonium chloride water solution (100 mL), water (100 mL) and brine (50 mL). The organics were dried over sodium sulfate and filtered through a 3 cm layer of silica gel which was washed with 1:1 ethyl acetate and dichloromethane mixture (100 mL). The combined organics were concentrated down under reduced pressure to afford 21.7 g of the crude product (as a mixture of both isomers on phosphorus based on the NMR). The solids were dissolved in minimum amount of boiling diisopropyl ether and the mixture was vigorously stirred at room temperature overnight. The solid product was filtered off and washed with cold diisopropyl ether (2×20 mL) and hexane (3×40 mL) to afford the product (a single isomer at phosphorus based on the NMR). ¹H NMR (400 MHz, DMSO-d₆) δ 7.47-7.36 (m, 2H), 7.30-7.20 (m, 3H), 6.92 (dd, J=14.2, 9.9 Hz, 1H), 4.21-4.08 (m, 2H), 4.07-3.92 (m, 1H), 3.62-3.56 (m, 2H), 3.53-3.47 (m, 2H), 3.45-3.36 (m, 4H), 1.29 (d, J=7.1 Hz, 3H), 1.07 (t, J=7.0 Hz, 3H). ¹⁹F NMR (376 MHz, DMSO-d₆) δ −154.24 (d, J=21.5 Hz, 2F), −160.86 (t, J=23.1 Hz, 1F), −163.68 (t, J=21.7 Hz, 2F). ³¹P NMR (162 MHz, DMSO-d₆) δ 0.40. LCMS: MS m/z=528.06 [M+1], t_{R}=1.64 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-0.2 min 2% acetonitrile, 0.2 min-1.5 min 2-100% acetonitrile, 1.5 min-2.2 min 100% acetonitrile, 2.2 min-2.4 min 100%-2% acetonitrile, 2.4 min-2.5 min 2% acetonitrile at 2 μL/min.

### Intermediate 74. cyclohexyl ((S)-(4-nitrophenoxy)(phenoxy)phosphoryl)-L-alaninate

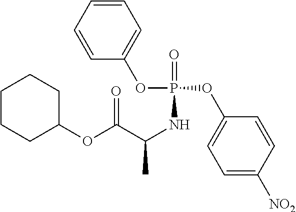 [SMILES: C[C@H](N[P@](=O)(Oc1ccccc1)Oc1ccc([N+](=O)[O-])cc1)C(=O)OC1CCCCC1]

Intermediate 25 (1.3 g, 2.90 mmol) was suspended in diisopropyl ether (3 mL) and para-nitrophenol (14 mg, 0.1 mmol) and DBU (0.05 mL, 0.335 mmol) were added at RT. The resulting mixture was stirred for 4 h and 1 N aqueous hydrochloric acid solution and ethyl acetate were added. The organic layer was split and was dried over anhydrous sodium sulfate and concentrated under reduced pressure. The resulting residue was taken up into diisopropyl ether (2 mL) and was sonicated to disperse the solids. The solids were collected by vacuum filtration to afford the product. ¹H NMR (400 MHz, Methanol-d₄) δ 8.27 (d, J=9.1 Hz, 2H), 7.51-7.44 (m, 2H), 7.38 (dd, J=8.6, 7.2 Hz, 2H), 7.28-7.17 (m, 3H), 4.68 (dt, J=8.9, 4.6 Hz, 1H), 4.02 (dq, J=9.9, 7.2 Hz, 1H), 1.80-1.64 (m, 5H), 1.52 (s, 1H), 1.57-1.46 (m, 1H), 1.44-1.22 (m, 9H). ³¹P NMR (162 MHz, Methanol-d₄) δ −1.32. MS m/z=449 (M+H)⁺.

### B. Compounds

### Example 1. (S)-isopropyl 2-(((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

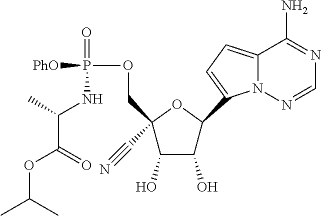 [SMILES: CC(C)OC(=O)[C@H](C)N[P@](=O)(OC[C@@]1(C#N)O[C@@H](c2ccc3c(N)ncnn23)[C@H](O)[C@@H]1O)Oc1ccccc1]

Intermediate 1 (50 mg, 0.172 mmol) and Intermediate 18 (84 mg, 0.206 mmol) were mixed in anhydrous N,N-dimethylformamide (2 mL). Magnesium chloride (36 mg, 0.378 mmol) was added in one portion. The reaction mixture was heated at 50° C. N,N-Diisopropylethylamine (75 μL, 0.43 mmol) was added, and the reaction was stirred for 4.5 hrs at 50° C. The reaction mixture was cooled, diluted with ethyl acetate (30 mL) and washed with 5% aqueous citric acid solution (10 mL) and then brine (10 mL). The organic layer was dried over anhydrous sodium sulfate and concentrated under reduced pressure. The crude product was purified via SiO₂ column chromatography (4 g SiO₂ Combiflash HP Gold Column 0-2-5% methanol/dichloromethane) to afford the product. ¹H NMR (400 MHz, Methanol-d₄) δ 7.79 (s, 1H), 7.36-7.25 (m, 2H), 7.25-7.12 (m, 3H), 6.84 (d, J=4.5 Hz, 1H), 6.73 (d, J=4.5 Hz, 1H), 5.49 (d, J=5.1 Hz, 1H), 4.91-4.84 (m, 1H), 4.62 (dd, J=5.6, 5.0 Hz, 1H), 4.47 (d, J=5.6 Hz, 1H), 4.45-4.30 (m, 2H), 3.85 (dq, J=10.0, 7.1 Hz, 1H), 1.25 (d, J=7.2 Hz, 3H), 1.15 (t, J=6.4 Hz, 6H). ³¹P NMR (162 MHz, Methanol-d₄) δ 3.31. MS m/z=561.0 [M+1], 559.0 [M−1].

### Example 2. (2S)-cyclobutylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

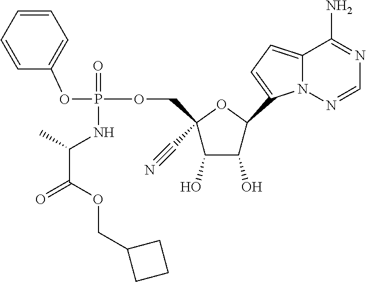 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCC1]

Intermediate 2 (50 mg, 0.116 mmol) and Intermediate 15 (60 mg, 0.139 mmol) were dissolved in anhydrous tetrahydrofuran (3 mL). Magnesium chloride (17 mg, 0.174 mmol) was added in one portion. Reaction was warmed to 60° C. and stirred for 20 min. N,N-Diisopropylethylamine (50 μL, 0.29 mmol) was added, and the reaction was stirred at 60° C. for 17 h. The reaction was cooled to room temperature, diluted with ethyl acetate (30 mL) and washed with 5% aqueous sodium carbonate solution (3×20 mL) and then brine (20 mL), dried over anhydrous sodium sulfate and concentrated under reduced pressure. Residue was dissolved in acetonitrile (2 mL) and stirred in an ice bath. 12 M hydrochloric acid (330 μL) was added dropwise and stirred for 20 h. The reaction was diluted with ethyl acetate (30 mL) and cooled in an ice bath. 1 N sodium hydroxide solution was added dropwise to give pH of 10. Organic layer was collected and washed with saturated aqueous sodium bicarbonate solution (10 mL) and then brine (10 mL). Organic layer was dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d4) δ 7.79 (m, 1H), 7.37-7.10 (m, 5H), 6.84 (dd, J=4.5, 2.3 Hz, 1H), 6.73 (dd, J=4.5, 2.4 Hz, 1H), 5.53-5.45 (m, 1H), 4.62 (q, J=5.5 Hz, 1H), 4.54-4.28 (m, 3H), 4.10-3.80 (m, 3H), 2.65-2.45 (m, 1H), 2.08-1.62 (m, 6H), 1.26 (d, J=7.2 Hz, 3H). ³¹P NMR (162 MHz, Methanol-d₄) δ 3.25, 3.24. MS m/z=587.2 [M+1], 585.2 [M−1].

### Example 3. (2S)-ethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-3-phenylpropanoate

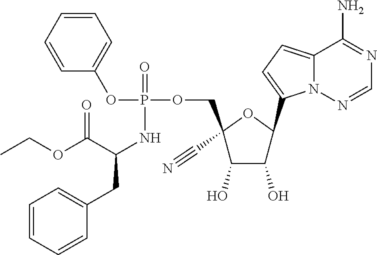 [SMILES: CCOC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

To a mixture of Intermediate 4 (52.0 mg, 0.121 mmol), Intermediate 19 (68.0 mg, 0.145 mmol), and magnesium chloride (17.2 mg, 0.181 mmol) was added THF (1.0 mL) at RT. The resulting suspension was warmed to 50° C., and was allowed to stir for 10 min. N,N-Diisopropylethylamine (0.052 mL, 0.301 mmol) was then added and the resulting mixture was stirred at 50° C. for 30 min. The reaction mixture was then allowed to cool to RT, and concentrated aqueous hydrochloric acid solution (12 M, 0.200 mL, 2.4 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.27 min, MS m/z=623.00 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.88 (m, 3H), 7.37-6.84 (m, 12H), 6.71 (t, J=4.2 Hz, 2H), 6.22 (ddd, J=23.7, 12.9, 10.5 Hz, 1H), 5.36 (dd, J=9.2, 6.1 Hz, 1H), 4.39 (s, 1H), 4.16 (dd, J=16.2, 5.3 Hz, 1H), 4.09-3.83 (m, 5H), 2.93 (dt, J=14.3, 7.3 Hz, 1H), 2.78 (m, 1H), 1.01 (t, 7.1 Hz, 3H). ³¹P NMR (162 MHz, DMSO-d₆) δ 3.85 (s), 2.86 (s).

### Example 4. (2S)-cyclohexyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

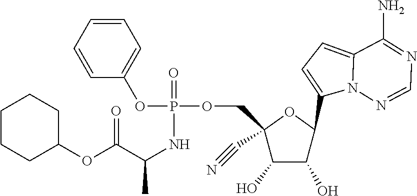 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

To a mixture of Intermediate 4 (99 mg, 0.30 mmol), Intermediate 25 (201 mg, 0.45 mmol), and MgCl₂ (43 mg, 0.45 mmol) in DMF (4 mL) was added N,N-diisopropylethylamine (0.13 mL, 0.75 mmol) dropwise at room temperature. The resulting mixture was stirred at room temperature for 15 h and purified by preparative HPLC (Phenominex Synergi 4 u Hydro-RR 80 Å 150×30 mm column, 10-100% acetonitrile/water gradient) to give an intermediate, which was dissolved in ACN (3 mL) and c-HCl (0.1 mL) was added. The resulting mixture was stirred at 50° C. for 2 h, cooled, and purified by preparative HPLC (Phenominex Synergi 4 u Hydro-RR 80 Å 150×30 mm column, 10-80% acetonitrile/water gradient) to give the product. ¹H NMR (400 MHz, Methanol-d4) δ 7.80 (s, 0.5H), 7.78 (s, 0.5H), 7.42-7.05 (m, 5H), 6.84 (m, 1H), 6.73 (m, 1H), 5.50 (m, 1H), 4.64 (m, 2H), 4.57-4.25 (m, 3H), 3.86 (m, 1H), 1.91-1.61 (m, 4H), 1.61-1.09 (m, 9H). ³¹P NMR (162 MHz, Methanol-d4) δ 3.3. MS m/z=601 (M+H)⁺.

Separation of the Diastereomers. The product was purified via chiral preparatory HPLC (Chiralpak IA, 150×4.6 mm, Heptane 70% Ethanol 30%).

### Example 5. cyclohexyl ((R)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

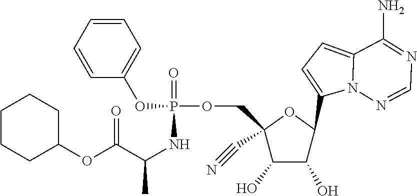 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCCCC1]

First Eluting Diastereomer of Example 4: ¹H NMR (400 MHz, Methanol-d₄) δ 7.78 (s, 1H), 7.34-7.23 (m, 2H), 7.19-7.10 (m, 3H), 6.85 (d, J=4.5 Hz, 1H), 6.73 (d, J=4.5 Hz, 1H), 5.51 (d, J=5.0 Hz, 1H), 4.69 (td, J=8.8, 4.2 Hz, 1H), 4.62 (t, J=5.3 Hz, 1H), 4.53-4.44 (m, 2H), 4.36 (dd, J=10.9, 5.2 Hz, 1H), 3.86 (dq, J=9.4, 7.1 Hz, 1H), 1.85-1.62 (m, 4H), 1.58-1.20 (m, 9H). ³¹P NMR (162 MHz, Methanol-d₄) δ 3.31.

### Example 6. cyclohexyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

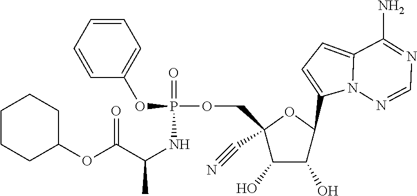 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]

Second Eluting Diastereomer of Example 4: ¹H NMR (400 MHz, Methanol-d₄) δ 7.80 (s, 1H), 7.37-7.27 (m, 2H), 7.26-7.13 (m, 3H), 6.84 (d, J=4.5 Hz, 1H), 6.73 (d, J=4.5 Hz, 1H), 5.49 (d, J=5.0 Hz, 1H), 4.71-4.56 (m, 2H), 4.46 (d, J=5.6 Hz, 1H), 4.45-4.30 (m, 2H), 3.87 (dq, J=10.0, 7.1 Hz, 1H), 1.80-1.61 (m, 4H), 1.55-1.21 (m, 9H). ³¹P NMR (162 MHz, Methanol-d₄) δ 3.31.

### Example 7

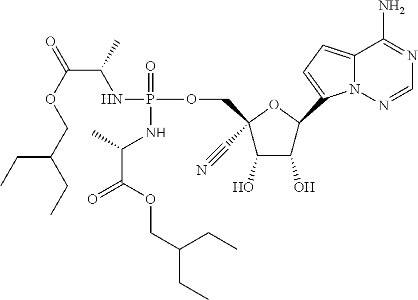 [SMILES: CCC(CC)COC(=O)[C@H](C)NP(=O)(N[C@@H](C)C(=O)OCC(CC)CC)OC[C@@]1(C#N)O[C@@H](c2ccc3c(N)ncnn23)[C@H](O)[C@@H]1O]

Intermediate 2 (60 mg, 0.139 mmol) was dissolved in anhydrous tetrahydrofuran (2 mL). Phosphorus oxychloride (25 μL, 0.278 mmol) was added in one portion and stirred for 30 mins. More phosphorus oxychloride (100 μL) was added and stirred for 30 mins. (S)-2-ethylbutyl 2-aminopropanoate hydrochloride (87 mg, 0.417 mmol) and triethylamine (116 μL, 0.834 mmol) were added and stirred for 30 mins. More (S)-2-ethylbutyl 2-aminopropanoate hydrochloride (500 mg) was added. Triethylamine was added to give pH of 9. Reaction was stirred for 16 hrs, diluted with ethyl acetate (20 mL) and washed with saturated aqueous sodium bicarbonate solution (2×20 mL), 5% aqueous citric acid solution (20 mL) followed with brine (20 mL). Organic layer was dried over anhydrous sodium sulfate and concentrated under reduced pressure. Residue was dissolved in acetonitrile (2 mL), 12 M hydrochloric acid (400 uL) was added and the mixture was stirred for 4 hrs. Reaction was diluted with ethyl acetate (30 mL) and washed with saturated aqueous sodium bicarbonate solution (2×10 mL) followed with brine (10 mL). Organic layer was dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.82 (s, 1H), 6.86 (d, J=4.5 Hz, 1H), 6.77 (d, J=4.5 Hz, 1H), 5.50 (d, J=4.8 Hz, 1H), 4.64-4.57 (m, 1H), 4.49 (d, J=5.7 Hz, 1H), 4.31 (dd, J=11.1, 7.1 Hz, 1H), 4.21 (dd, J=11.1, 5.8 Hz, 1H), 4.11-3.94 (m, 4H), 3.94-3.84 (m, 2H), 1.58-1.29 (m, 10H), 0.98-0.82 (m, 18H). ³¹P NMR (162 MHz, Methanol-d₄) δ 13.61. MS m/z=682.1 [M+1], 680.1 [M−1].

### Example 8. (2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

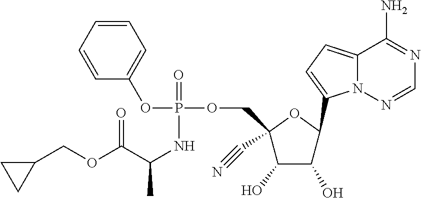 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CC1]

To a mixture of Intermediate 4 (70 mg, 0.211 mmol), Intermediate 20 (133 mg, 0.32 mmol), and MgCl₂ (30 mg, 0.32 mmol) in THF (3 mL) was added N,N-diisopropylethylamine (0.092 mL, 0.53 mmol) dropwise at room temperature. The resulting mixture was stirred at 60° C. for 15 h, diluted with EtOAc, washed with water and brine, dried with sodium sulfate, and concentrated in vacuo. The resulting residue was dissolved in ACN (3 mL) and c-HCl (0.3 mL) was added. The mixture was stirred at room temperature for 2 h and purified by preparative HPLC (Phenominex Synergi 4 u Hydro-RR 80 Å 150×30 mm column, 10-70% acetonitrile/water gradient) to give the product. ¹H NMR (400 MHz, Methanol-d4) δ 7.79 (m, 1H), 7.39-7.10 (m, 5H), 6.85 (m, 1H), 6.73 (m, 1H), 5.50 (m, 1H), 4.62 (m, 1H), 4.58-4.24 (m, 3H), 4.00-3.69 (m, 3H), 1.27 (m, 3H), 1.17-0.95 (m, 1H), 0.49 (m, 2H), 0.29-0.15 (m, 2H). ³¹P NMR (162 MHz, Methanol-d4) δ 3.29, 3.22. MS m/z 573 (M+H)⁺.

Separation of the (S) and (R) diastereomers. The product was separated by chiral preparatory HPLC (Chiralpak IA, 150×4.6 mm, Heptane 80% Ethanol 20%) to afford the diastereomers:

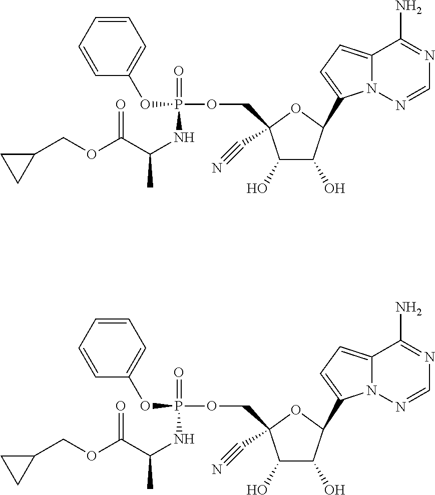 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CC1 ; C[C@H](N[P@](=O)(OC[C@@]1(C#N)O[C@@H](c2ccc3c(N)ncnn23)[C@H](O)[C@@H]1O)Oc1ccccc1)C(=O)OCC1CC1]

### Example 9. First Eluting Diastereomer of Example 8

¹H NMR (400 MHz, Methanol-d4) δ 7.78 (s, 1H), 7.29 (dd, J=8.7, 7.1 Hz, 2H), 7.22-7.06 (m, 3H), 6.85 (d, J=4.5 Hz, 1H), 6.73 (d, J=4.5 Hz, 1H), 5.50 (d, J=5.0 Hz, 1H), 4.63 (t, J=5.3 Hz, 1H), 4.55-4.44 (m, 2H), 4.36 (dd, J=10.9, 5.1 Hz, 1H), 3.97-3.77 (m, 3H), 1.26 (dd, J=7.2, 1.2 Hz, 3H), 1.15-1.04 (m, 1H), 0.58-0.45 (m, 2H), 0.32-0.18 (m, 2H). ³¹P NMR (162 MHz, Methanol-d4) δ 3.30.

### Example 10. Second eluting diastereomer of Example 8

¹H NMR (400 MHz, Methanol-d4) δ 7.80 (s, 1H), 7.38-7.26 (m, 2H), 7.29-7.11 (m, 3H), 6.84 (d, J=4.5 Hz, 1H), 6.73 (d, J=4.5 Hz, 1H), 5.49 (d, J=5.1 Hz, 1H), 4.62 (t, J=5.3 Hz, 1H), 4.47 (d, J=5.6 Hz, 1H), 4.42 (dd, J=10.9, 6.3 Hz, 1H), 4.34 (dd, J=10.9, 5.4 Hz, 1H), 3.98-3.82 (m, 2H), 3.78 (dd, J=11.4, 7.3 Hz, 1H), 1.27 (dd, J=7.2, 1.1 Hz, 3H), 1.11-0.98 (m, 1H), 0.52-0.45 (m, 2H), 0.25-0.14 (m, 2H). ³¹P NMR (162 MHz, Methanol-d4) δ 3.23.

### Example 11. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl pivalate

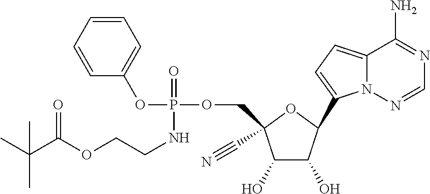 [SMILES: CC(C)(C)C(=O)OCCNP(=O)(OC[C@@]1(C#N)O[C@@H](c2ccc3c(N)ncnn23)[C@H](O)[C@@H]1O)Oc1ccccc1]

To a mixture of Intermediate 4 (22.0 mg, 0.066 mmol), Intermediate 21 (28.1 mg, 0.066 mmol), and magnesium chloride (6.3 mg, 0.166 mmol) was added acetonitrile (0.50 mL) at RT. The resulting suspension was warmed to 50° C., and was allowed to stir for 5 min. N,N-Diisopropylethylamine (0.03 mL, 0.066 mmol) was then added and the resulting mixture was stirred at 50° C. for 1 h. The reaction mixture was then allowed to cool to RT, and concentrated aqueous hydrochloric acid solution (12 M, 0.077 mL, 0.93 mmol) was added. After 1 h, the reaction mixture was diluted with saturated aqueous sodium carbonate solutions (20 mL) and ethyl acetate (20 mL). The layers were split and the organic layer was washed with brine (20 mL), was dried over anhydrous sodium sulfate, and was concentrated under reduced pressure. The crude residue was purified by preparatory HPLC (Phenominex Synergi 4 u Hydro-RR 80 Å 150×30 mm column, 40-100% acetonitrile/water gradient) to afford the product. MS m/z=575.00 [M+H].

Separation of the (S) and (R) Diastereomers. The product was purified via chiral preparatory HPLC (Chiralpak IC, 150×4.6 mm, Heptane 80% Ethanol 20%) to afford the diastereomers:

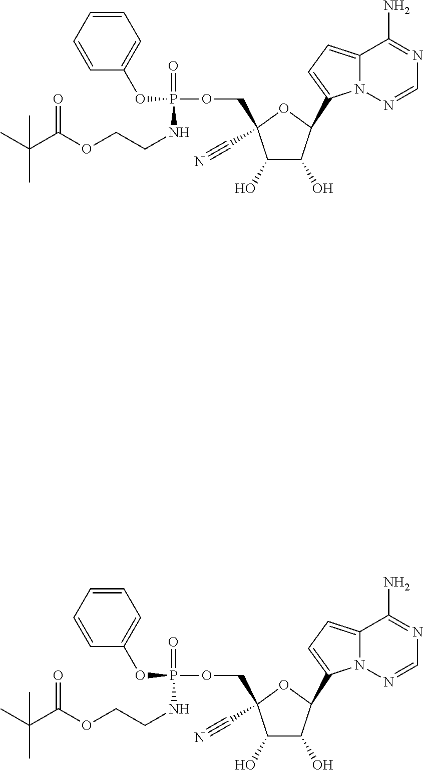 [SMILES: CC(C)(C)C(=O)OCCN[P@@](=O)(OC[C@@]1(C#N)O[C@@H](c2ccc3c(N)ncnn23)[C@H](O)[C@@H]1O)Oc1ccccc1 ; CC(C)(C)C(=O)OCCN[P@](=O)(OC[C@@]1(C#N)O[C@@H](c2ccc3c(N)ncnn23)[C@H](O)[C@@H]1O)Oc1ccccc1]

### Example 12. First Eluting Diastereomer of Example 11

¹H NMR (400 MHz, CD₃OD) δ 7.80 (s, 1H), 7.33 (t, J=7.9 Hz, 2H), 7.27-7.13 (m, 3H), 6.84 (d, J=4.5 Hz, 1H), 6.74 (d, J=4.5 Hz, 1H), 5.50 (d, J=4.9 Hz, 1H), 4.63 (t, J=5.3 Hz, 1H), 4.47 (d, J=5.6 Hz, 1H), 4.45-4.29 (m, 2H), 3.96 (t, J=5.7 Hz, 2H), 3.14 (dt, J=11.8, 5.7 Hz, 2H), 1.12 (s, 9H). ³¹P NMR (162 MHz, CD₃OD) δ 5.24 (s). MS m/z=575.00 [M+H].

### Example 13. Second Eluting Diastereomer of Example 11

¹H NMR (400 MHz, CD₃OD) δ 7.77 (s, 1H), 7.32-7.25 (m, 2H), 7.19-7.12 (m, 3H), 6.85 (d, J=4.5 Hz, 1H), 6.72 (d, J=4.5 Hz, 1H), 5.50 (d, J=4.9 Hz, 1H), 4.62 (t, J=5.2 Hz, 1H), 4.49 (d, J=5.5 Hz, 1H), 4.42 (dd, J=10.9, 6.1 Hz, 1H), 4.33 (dd, J=10.9, 5.5 Hz, 1H), 3.99 (d, J=5.3 Hz, 1H), 3.19-3.10 (m, 2H), 1.15 (s, 9H). ³¹P NMR (162 MHz, CD₃OD) δ 5.05 (br s). MS m/z=575.00 [M+H].

### Example 14

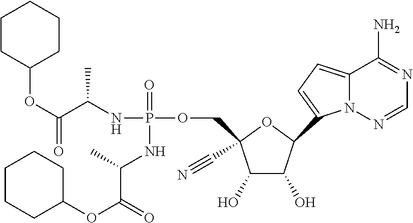 [SMILES: C[C@H](NP(=O)(N[C@@H](C)C(=O)OC1CCCCC1)OC[C@@]1(C#N)O[C@@H](c2ccc3c(N)ncnn23)[C@H](O)[C@@H]1O)C(=O)OC1CCCCC1]

Intermediate 4 (50 mg, 0.15 mmol) and Intermediate 60 (95 mg, 0.18 mmol) were dissolved in anhydrous tetrahydrofuran (3 mL). Magnesium chloride (21 mg, 0.225 mmol) was added in one portion. Reaction was warmed to 50° C. and stirred for 30 mins. N,N-Diisopropylethylamine (65 μL, 0.375 mmol) was added, and the reaction was stirred for 16 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred in an ice bath for 60 mins. Reaction was diluted with ethyl acetate (30 mL) and cooled in an ice bath. Saturated aqueous sodium bicarbonate solution was added dropwise to give pH of 16. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.82 (s, 1H), 6.86 (d, J=4.5 Hz, 1H), 6.77 (d, J=4.5 Hz, 1H), 5.51 (d, J=4.8 Hz, 1H), 4.70 (m, 2H), 4.61 (dd, J=5.7, 4.9 Hz, 1H), 4.49 (d, J=5.7 Hz, 1H), 4.36-4.17 (m, 2H), 3.86 (m, 2H), 1.88-1.63 (m, 8H), 1.58-1.25 (m, 18H). ³¹P NMR (162 MHz, Methanol-d₄) δ 13.64. MS m/z=678.1 [M+1], 676.2 [M−1].

### Example 15. (S)-1-methylpyrrolidin-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

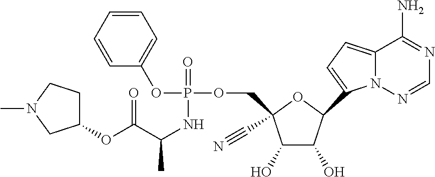 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCN(C)C1]

Intermediate 4 (99 mg, 0.3 mmol) and Intermediate 23 (162 mg, 0.36 mmol) were mixed and dissolved in 2 mL of anhydrous THF. Magnesium chloride (86 mg, 0.9 mmol) was added in one portion. DIPEA (131 uL, 0.75 mmol) was added, and the reaction was stirred at 50° C. for 5 hrs.

Reaction was diluted with EtOAc (15 mL) and washed with water (4×15 mL) and then with brine (5 mL). Organic extract was dried over anhydrous sodium sulfate and concentrated under reduced pressure. The crude residue was purified via SiO₂ column chromatography (4 g SiO₂ Combiflash HP Gold Column, 0-5-10-20% methanol/DCM). Fractions having the desired product were combined and concentrated under reduced pressure. Residue was dissolved in MeCN (7 mL) and stirred in an ice bath. Concentrate aqueous hydrochloric acid (500 uL) was added dropwise. Reaction was stirred in an ice bath for 2 hrs. Reaction was diluted with EtOAc (30 mL) and added saturated aqueous sodium bicarbonate solution (30 mL). Mixture was stirred for 10 mins. Organic extract was collected and aqueous portion was extracted with EtOAc (2×10 mL). Organic extracts were combined, dried over anhydrous sodium sulfate and concentrated under reduced pressure. Residue was dissolved in MeCN and water and freeze-dried to give the product. ¹H NMR (400 MHz, methanol-d₄) δ 7.79 (m, 1H), 7.39-7.26 (m, 2H), 7.26-7.10 (m, 3H), 6.84 (m, 1H), 6.73 (m, 1H), 5.49 (m, 1H), 5.18-4.98 (m, 1H), 4.62 (m, 1H), 4.55-4.28 (m, 3H), 3.89 (m, 1H), 2.78 (m, 1H), 2.69-2.54 (m, 2H), 2.32 (m, 4H), 2.23-2.08 (m, 1H), 1.78 (m, 1H), 1.27 (m, 3H). ³¹P NMR (162 MHz, methanol-d₄) δ 3.28, 3.14. LCMS: MS m/z=602.2 [M+1], 600.2 [M−1], t_{R}=0.99 min; LC system: Thermo Dionex Ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=1.84 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.868 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 16. trans-4-(trifluoromethyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

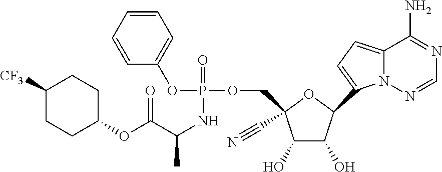 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

The product was obtained from Intermediate (129 mg, 0.25 mmol) and Intermediate 4 (55 mg, 0.25 mmol) in a manner similar to that described for Example 3. ¹H NMR (400 MHz, Methanol-d4) δ 7.78 (m, 1H), 7.32 (m, 2H), 7.25-7.12 (m, 3H), 6.84 (m, 1H), 6.73 (m, 1H), 5.50 (dd, J=5.1, 1.9 Hz, 1H), 4.69-4.49 (m, 2H), 4.49-4.32 (m, 3H), 3.93-3.75 (m, 1H), 2.23-1.71 (m, 5H), 1.44-1.20 (m, 7H). ³¹P NMR (162 MHz, Methanol-d4) δ 3.28, 3.22. ¹⁹F NMR (377 MHz, Methanol-d4) δ −75.31-75.40 (m). MS m/z=669 [M+H].

The product was separated by SFC using 30% ethanol (AD-H4.6X100m column).

### Example 17. trans-4-(trifluoromethyl)cyclohexyl ((R)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

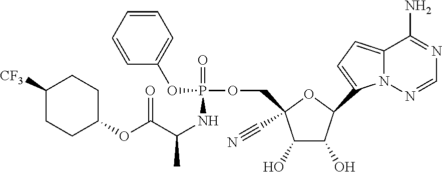 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

First eluting diastereomer of Example 16: ¹H NMR (400 MHz, Methanol-d4) δ 7.78 (s, 1H), 7.29 (t, J=7.9 Hz, 2H), 7.21-7.10 (m, 3H), 6.85 (d, J=4.5 Hz, 1H), 6.73 (d, J=4.5 Hz, 1H), 5.50 (d, J=5.0 Hz, 1H), 4.62 (q, J=5.2 Hz, 2H), 4.52 (d, J=5.6 Hz, 1H), 4.47 (dd, J=10.9, 6.0 Hz, 1H), 4.35 (dd, J=10.9, 5.1 Hz, 1H), 3.83 (dq, J=9.1, 7.1 Hz, 1H), 2.10 (m, 1H), 1.96 (m, 4H), 1.38 (m, 4H), 1.23 (dd, J=7.1, 1.2 Hz, 3H). ³¹P NMR (162 MHz, Methanol-d4) δ 3.29. ¹⁹F NMR (377 MHz, Methanol-d4) δ −75.41 (d, J=8.6 Hz).

### Example 18. trans-4-(trifluoromethyl)cyclohexyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

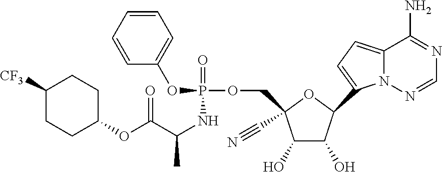 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

Second eluting diastereomer of Example 16: ¹H NMR (400 MHz, Methanol-d4) δ 7.79 (s, 1H), 7.39-7.27 (m, 2H), 7.29-7.13 (m, 3H), 6.84 (d, J=4.5 Hz, 1H), 6.73 (d, J=4.5 Hz, 1H), 5.50 (d, J=5.0 Hz, 1H), 4.58 (m, 2H), 4.43 (m, 2H), 4.35 (dd, J=10.9, 5.5 Hz, 1H), 3.86 (dq, J=9.9, 7.4 Hz, 1H), 2.14-1.81 (m, 5H), 1.32 (m, 4H), 1.24 (dd, J=7.1, 1.0 Hz, 3H). ³¹P NMR (162 MHz, Methanol-d4) δ 3.22. ¹⁹F NMR (377 MHz, Methanol-d4) δ −75.33 (d, J=8.5 Hz).

The product was also obtained from Intermediate 29 (701 mg, 1.36 mmol) and Intermediate 4 (300 mg, 0.91 mmol) in a manner similar to that described for Example 3.

### Example 19. 1-methylpiperidin-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

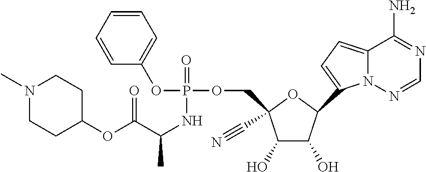 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCN(C)CC1]

To a mixture of Intermediate 4 (70 mg, 0.211 mmol), Intermediate 30 (293 mg, 0.317 mmol), and MgCl₂ (30 mg, 0.317 mmol) in THF (3 mL) was added N,N-diisopropylethylamine (0.09 mL, 0.528 mmol) dropwise at room temperature. The resulting mixture was stirred at 50° C. for 2 h, purified by preparative HPLC (Phenomenex Gemini-NX 10μ C18 110° A 250×30 mm column, 0%-100% acetonitrile/water gradient in 25 min run) to give an acetonide intermediate, which was dissolved in acetonitrile (3 mL) and c-HCl (0.5 mL) was added. The resulting mixture was stirred for 2 h, concentrated in vacuo, and lyophilized to afford the product. ¹H NMR (400 MHz, Methanol-d4) δ 7.85 (m, 1H), 7.41-7.12 (m, 5H), 6.97 (m, 1H), 6.79 (m, 1H), 5.51 (m, 1H), 5.04 (m, 1H), 4.62 (m, 1H), 4.53-4.31 (m, 3H), 3.98 (m, 1H), 3.66 (m, 5H), 2.82 (m, 2H), 2.10 (m, 4H), 1.30 (m, 3H). ³¹P NMR (162 MHz, Methanol-d4) δ 3.32, 3.10. LCMS: MS m/z=616.24 [M+1-HCl]; t_{R}=0.54 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Example 20. (tetrahydro-2H-pyran-4-yl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy) phosphoryl)-L-alaninate

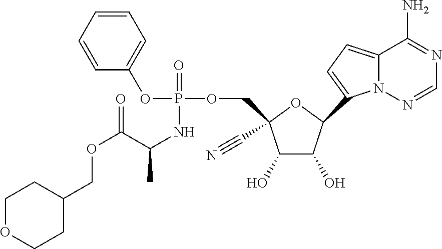 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCOCC1]

Intermediate 4 (50 mg, 0.15 mmol) and Intermediate 31 (84 mg, 0.18 mmol) were mixed and dissolved in anhydrous THF (5 mL). Magnesium chloride (86 mg, 0.906 mmol) was added in one portion and the reaction was stirred at 50° C. for 10 mins. DIPEA (158 uL, 0.906 mmol) was added and the reaction was stirred at 50° C. for 2 hrs. More magnesium chloride (50 mg) was added and stirred at 50° C. for 16 hrs.

Reaction was diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 1 hr. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions containing the desired product were combined and concentrated under reduced pressure to give oil which was then dissolved in MeCN and water and freeze-dried to afford the product. ¹H NMR (400 MHz, Methanol-d4) δ 7.79 (m, 1H), 7.41-7.07 (m, 5H), 6.84 (m, 1H), 6.73 (m, 1H), 5.57-5.40 (m, 1H), 4.62 (m, 1H), 4.56-4.28 (m, 3H), 3.87 (m, 5H), 3.31 (m, 2H), 1.94-1.72 (m, 1H), 1.63-1.46 (m, 2H), 1.34-1.16 (m, 5H). ³¹P NMR (162 MHz, Methanol-d4) δ 3.23 (s), 3.19 (s). MS m/z=617.1 [M+1]; 615.0 [M−1].

### Example 21. trans-4-(tert-butyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

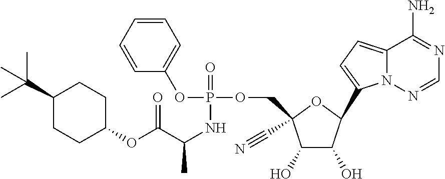 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

The product was obtained from Intermediate 32 (116 mg, 0.23 mmol) and Intermediate 4 (51 mg, 0.15 mmol) in a manner similar to that described for Example 3. ¹H NMR (400 MHz, Methanol-d4) δ 7.79 (m, 1H), 7.32 (m, 2H), 7.25-7.18 (m, 2H), 7.19-7.10 (m, 1H), 6.85 (m, 1H), 6.76-6.69 (m, 1H), 5.51 (m, 1H), 4.65-4.57 (m, 1H), 4.51 (m, 1H), 4.47-4.39 (m, 2H), 4.35 (m, 1H), 3.93-3.76 (m, 1H), 1.93 (m, 2H), 1.74 (m, 2H), 1.24 (m, 5H), 1.13-0.89 (m, 3H), 0.84 (m, 9H). ³¹P NMR (162 MHz, Methanol-d4) δ 3.35, 3.28. MS m/z=657 [M+H].

The mixture was separated by Chiralpak SFC (Chiralpak ID 21×250 mm column, 30% methanol).

### Example 22. trans-4-(tert-butyl)cyclohexyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

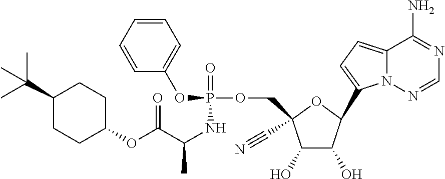 [SMILES: C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

First eluting diastereomer of Example 21. ¹H NMR (400 MHz, Methanol-d4) δ 7.80 (s, 1H), 7.33 (t, J=7.8 Hz, 2H), 7.26-7.12 (m, 3H), 6.85 (d, J=4.5 Hz, 1H), 6.74 (d, J=4.5 Hz, 1H), 5.51 (d, J=4.9 Hz, 1H), 4.59 (t, J=5.3 Hz, 1H), 4.51 (tt, J=11.3, 4.5 Hz, 1H), 4.46-4.39 (m, 2H), 4.35 (dd, J=10.9, 5.6 Hz, 1H), 3.89-3.81 (m, 1H), 1.99-1.86 (m, 2H), 1.75 (t, J=12.0 Hz, 2H), 1.31-1.18 (m, 5H), 1.12-0.89 (m, 3H), 0.83 (s, 9H). ³¹P NMR (162 MHz, Methanol-d4) δ 3.25.

### Example 23. trans-4-(tert-butyl)cyclohexyl ((R)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

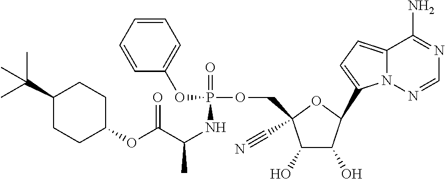 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(C)(C)C)CC1]

Second eluting diastereomer of Example 21. ¹H NMR (400 MHz, Methanol-d4) δ 7.78 (s, 1H), 7.29 (t, J=7.8 Hz, 2H), 7.18-7.11 (m, 3H), 6.85 (d, J=4.5 Hz, 1H), 6.73 (d, J=4.5 Hz, 1H), 5.51 (d, J=5.0 Hz, 1H), 4.62 (t, J=5.3 Hz, 1H), 4.59-4.43 (m, 3H), 4.36 (dd, J=10.9, 5.2 Hz, 1H), 3.82 (q, J=7.9 Hz, 1H), 1.94 (m, 2H), 1.79 (m, 2H), 1.36-1.20 (m, 5H), 1.13-0.94 (m, 3H), 0.85 (s, 9H). ³¹P NMR (162 MHz, Methanol-d4) δ 3.32.

### Example 24. 2-ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(benzyloxy)phosphoryl)-L-alaninate

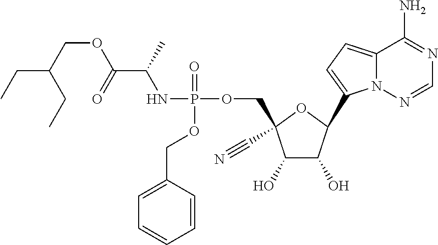 [SMILES: CCC(CC)COC(=O)[C@H](C)NP(=O)(OCc1ccccc1)OC[C@@]1(C#N)O[C@@H](c2ccc3c(N)ncnn23)[C@H](O)[C@@H]1O]

Acetonitrile (2.5 mL) was added to a mixture of Intermediate 4 (200 mg, 0.604 mmol), Intermediate 16 (280 mg, 0.604 mmol), and magnesium chloride (57 mg, 0.60 mmol) at RT. The mixture was heated to 50° C. for 5 min, and N,N-diisopropylethylamine (0.263 mL, 0.604 mmol) was added. After 22 h, the reaction mixture was allowed to cool to RT, and concentrated aqueous hydrochloric acid solution (0.5 mL) was added dropwise. After 1 h, the reaction mixture was diluted with ethyl acetate (100 mL) and the resulting mixture was washed with saturated aqueous sodium carbonate solution (50 mL) and brine (50 mL). The organic layer was dried over anhydrous sodium sulfate and concentrated under reduced pressure. The crude residue was subjected to silica gel chromatography eluting with 0-100% ethyl acetate in hexanes to afford the product. ¹H NMR (400 MHz, methanol-d₄) δ 7.78 (s, 0.6H), 7.75 (s, 0.4H), 7.40-7.26 (m, 5H), 6.85-6.80 (m, 1H), 6.75-6.69 (m, 1H), 5.54-5.48 (m, 2H), 5.06 (d, J=7.5 Hz, 1.2H), 4.99 (d, J=7.3 Hz, 0.8H), 4.64-4.58 (m, 1H), 4.52-4.46 (m, 1H), 4.41-4.20 (m, 2H), 4.07-3.77 (m, 2H), 1.54-1.21 (m, 8H), 0.95-0.77 (m, 6H). ³¹P NMR (162 MHz, methanol-d₄) δ 7.90 (s), 7.82 (s). LCMS: MS m/z=617.14 [M+1], t_{R}=1.26 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.057 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

### Example 25. 2-ethylbutyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

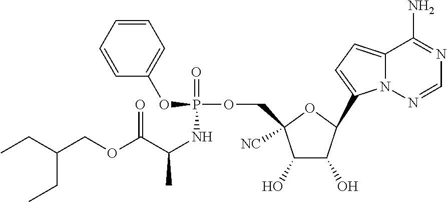 [SMILES: CCC(CC)COC(=O)[C@H](C)N[P@](=O)(OC[C@@]1(C#N)O[C@@H](c2ccc3c(N)ncnn23)[C@H](O)[C@@H]1O)Oc1ccccc1]

To a mixture of Intermediate 4 (700 mg, 2.113 mmol), Intermediate 17 (998 mg, 2.218 mmol), and magnesium chloride (302 mg, 3.169 mmol) was added tetrahydrofuran (8.5 mL) at room temperature followed by the addition of N,N-Diisopropylethylamine (0.92 mL, 5.282 mmol). The resulting mixture was stirred at 50° C. for 3 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified via SiO₂ column chromatography (80 g SiO₂ Combiflash HP Gold Column, 100% Dichloromethane-14% Methanol in dichloromethane as eluent). Pure material obtained was dissolved in an anhydrous acetonitrile (10 mL) and was cooled in an ice bath followed by the dropwise addition of concentrated hydrochloric acid (4 mL, 48 mmol). The reaction mixture was stirred at room temperature for 1 h. After 1 h the reaction mixture was cooled in an ice bath and was diluted with water. Neutralized the solution with 3 N sodium hydroxide and extracted with dichloromethane. Organic layer was separated, dried over sodium sulfate, filtered and concentrated. The residue obtained was purified by SiO₂ column chromatography (40 g SiO₂ Combiflash HP Gold Column, 100% Dichloromethane-20% Methanol in dichloromethane) to afford the product. ¹H NMR (400 MHz, Methanol-d4) δ 7.80 (s, 1H), 7.38-7.29 (m, 2H), 7.27-7.13 (m, 3H), 6.84 (d, J=4.5 Hz, 1H), 6.74 (d, J=4.5 Hz, 1H), 5.49 (d, J=5.0 Hz, 1H), 4.61 (t, J=5.3 Hz, 1H), 4.49-4.29 (m, 3H), 4.04-3.82 (m, 3H), 1.43 (dq, J=12.5, 6.1 Hz, 1H), 1.37-1.23 (m, 7H), 0.84 (td, J=7.5, 1.1 Hz, 6H). ³¹P NMR (162 MHz, Acetonitrile-d3) δ 2.73. MS m/z=603 [M+1].

### Example 26. ((1r,4S)-4-(trifluoromethyl)cyclohexyl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

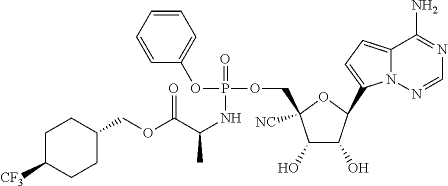 [SMILES: C[C@H](NP(=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1]

To a mixture of Intermediate 4 (0.06 g, 0.181 mmol), Intermediate 33 (0.115 g, 0.217 mmol), and magnesium chloride (0.028 g, 0.29 mmol) was added tetrahydrofuran (1.5 mL) at room temperature followed by the addition of N,N-Diisopropylethylamine (0.079 mL, 0.453 mmol). The resulting mixture was stirred at 50° C. for 3 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified via SiO₂ column chromatography (40 g SiO₂ Combiflash HP Gold Column, 100% Dichloromethane-14% Methanol in dichloromethane as eluent). Pure material obtained was dissolved in an anhydrous acetonitrile (2 mL) and was cooled in an ice bath followed by the dropwise addition of concentrated hydrochloric acid (0.1 mL, 1.2 mmol). The reaction mixture was stirred at room temperature for 1 h. After 1 h the reaction mixture was cooled in an ice bath and was diluted with saturated sodium bicarbonate solution (1 mL). The resulting mixture was purified by preparative HPLC (Phenominex Synergi 4 u Hydro-RR 80 Å 150×30 mm column, 15%-85% acetonitrile/water gradient in 30 min run) to afford the product. ¹H NMR (400 MHz, Acetonitrile-d3) δ 7.82 (d, J=3.1 Hz, 1H), 7.40-7.10 (m, 5H), 6.81-6.67 (m, 2H), 6.54 (s, 2H), 5.50 (t, J=5.0 Hz, 1H), 4.72-4.26 (m, 6H), 4.05-3.69 (m, 3H), 2.17-1.93 (m, 1H), 1.88 (dt, J=13.3, 3.6 Hz, 2H), 1.82-1.69 (m, 2H), 1.55 (dtq, J=12.0, 5.8, 3.0 Hz, 1H), 1.33-1.14 (m, 5H), 1.10-0.86 (m, 2H). ³¹P NMR (162 MHz, Acetonitrile-d3) δ 2.77, −2.68. ¹⁹F NMR (376 MHz, Acetonitrile-d3) δ −74.72 (d, J=8.7 Hz). MS m/z=683.20 [M+1].

Separation of the (S) and (R) Diastereomers. The product was purified via chiral preparatory HPLC (AD-H 5 um 21×250 mm, Heptane 70%, Ethanol 30%) to afford the diastereomers:

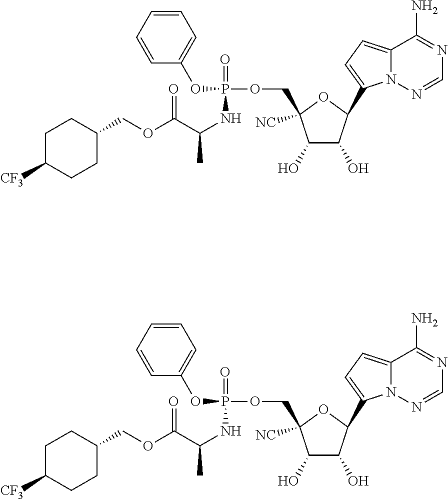 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1 ; C[C@H](N[P@](=O)(OC[C@@]1(C#N)O[C@@H](c2ccc3c(N)ncnn23)[C@H](O)[C@@H]1O)Oc1ccccc1)C(=O)OC[C@H]1CC[C@H](C(F)(F)F)CC1]

### Example 27. First Eluting Diastereomer of Example 26

¹H NMR (400 MHz, Acetonitrile-d3) δ 7.88 (s, 1H), 7.35 (t, J=7.8 Hz, 2H), 7.25-7.15 (m, 3H), 6.79-6.71 (m, 2H), 6.23 (s, 2H), 5.48 (d, J=4.9 Hz, 1H), 4.66-4.55 (m, 1H), 4.50 (t, J=6.0 Hz, 1H), 4.43 (dd, J=11.1, 6.5 Hz, 1H), 4.37-4.17 (m, 3H), 4.07-3.83 (m, 4H), 1.93 (d, J=13.5 Hz, 2H), 1.81 (d, J=13.5 Hz, 2H), 1.62 (d, J=6.2 Hz, 1H), 1.40-1.20 (m, 6H), 1.03 (q, J=12.9 Hz, 2H). ¹⁹F NMR (376 MHz, Acetonitrile-d3) δ −74.83 (d, J=8.8 Hz). ³¹P NMR (162 MHz, Acetonitrile-d3) δ 2.59. MS m/z=683.20 [M+1].

### Example 28. Second Eluting Diastereomer of Example 26

¹H NMR (400 MHz, Acetonitrile-d3) δ 7.89 (s, 1H), 7.37 (t, J=7.8 Hz, 2H), 7.31-7.14 (m, 3H), 6.75 (s, 2H), 6.24 (s, 2H), 5.47 (d, J=4.9 Hz, 1H), 4.58 (q, J=5.1 Hz, 1H), 4.48 (t, J=6.0 Hz, 1H), 4.43-4.20 (m, 4H), 4.05-3.73 (m, 4H), 1.91 (d, J=13.2 Hz, 2H), 1.78 (d, J=13.0 Hz, 2H), 1.65-1.47 (m, 1H), 1.39-1.19 (m, 6H), 1.01 (t, J=13.0 Hz, 2H). ¹⁹F NMR (376 MHz, Acetonitrile-d3) δ −74.83 (d, J=8.8 Hz). ³¹P NMR (162 MHz, Acetonitrile-d3) δ 2.67. MS m/z=683.20 [M+1].

### Example 29. Ethyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

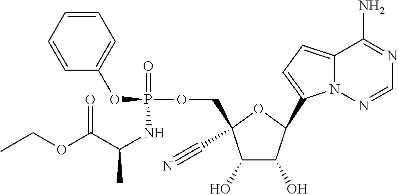 [SMILES: CCOC(=O)[C@H](C)N[P@](=O)(OC[C@@]1(C#N)O[C@@H](c2ccc3c(N)ncnn23)[C@H](O)[C@@H]1O)Oc1ccccc1]

To a mixture of Intermediate 4 (150 mg, 0.45 mmol), Intermediate 34 (298 mg, 0.68 mmol), and MgCl₂ (65 mg, 0.68 mmol) in THF (6 mL) was added N,N-diisopropylethylamine (0.20 mL, 1.13 mmol) dropwise. The resulting mixture was stirred at 50° C. for 2 h, cooled, diluted with EtOAc (150 mL), washed with brine (50 mL×2), dried, concentrated in vacuo, redissolved in acetonitrile (6 mL), and c-HCL (0.3 mL) added in ice bath. The resulting mixture was stirred for 1 h in ice bath and 1 h at room temperature, treated with saturated NaHCO₃ (2 mL), purified by HPLC (Phenomenex Gemini-NX 10ρ C18 110° A 250×30 mm column, 5-70% acetonitrile/water gradient in 25 min run) to afford the product. ¹H NMR (400 MHz, Methanol-d4) δ 7.80 (s, 1H), 7.31 (d, J=7.7 Hz, 2H), 7.25-7.14 (m, 3H), 6.84 (d, J=4.5 Hz, 1H), 6.73 (d, J=4.6 Hz, 1H), 5.49 (d, J=5.1 Hz, 1H), 4.62 (t, J=5.3 Hz, 1H), 4.46 (d, J=5.6 Hz, 1H), 4.40 (dd, J=10.9, 6.2 Hz, 1H), 4.33 (dd, J=10.9, 5.4 Hz, 1H), 4.11-3.98 (m, 2H), 3.87 (dd, J=9.9, 7.1 Hz, 1H), 1.25 (dd, J=7.1, 1.0 Hz, 3H), 1.16 (t, J=7.1 Hz, 3H). ³¹P NMR (162 MHz, Methanol-d4) δ 3.26. LCMS: MS m/z=547.12 [M+1]; t_{R}=0.76 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.03 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 30. cyclohexyl ((((2R,3S,4R,5S)-5-(4-aminopyrrolo[2,1-f][1,2,4] triazin-7-yl)-2-cyano-3,4-dihydroxytetrahydrofuran-2-yl)methoxy)(((S)-1-(2-ethylbutoxy)-1-oxopropan-2-yl)amino)phosphoryl)-L-alaninate

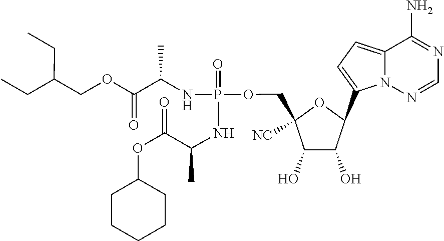 [SMILES: CCC(CC)COC(=O)[C@H](C)NP(=O)(N[C@@H](C)C(=O)OC1CCCCC1)OC[C@@]1(C#N)O[C@@H](c2ccc3c(N)ncnn23)[C@H](O)[C@@H]1O]

To a mixture of Intermediate 4 (65 mg, 0.196 mmol), Intermediate 59 (124 mg, 0.235 mmol), and MgCl₂ (40 mg, 0.42 mmol) in THF (2 mL) was added N,N-diisopropylethylamine (0.085 mL, 0.490 mmol) dropwise. The resulting mixture was stirred at about 50° C. for about 2 h, cooled, purified by preparative HPLC (Phenomenex Gemini-NX 10μ C18 110° A 250×30 mm column, 0%-100% acetonitrile/water gradient in 25 min run) to give an acetonide intermediate, which was dissolved in acetonitrile (2 mL) and c-HCl (0.1 mL) was added under icebath. The resulting mixture was then stirred under ice-bath for about 2 h and sat NaHCO₃ (2 mL) added slowly. The resulting mixture was then purified by preparative HPLC (Phenomenex Gemini-NX 10μ C18 110° A 250×30 mm column, 0%-80% acetonitrile/water gradient in 25 min run) to afford the product. ¹H NMR (400 MHz, Acetonitrile-d3) δ 7.86 (s, 1H), 6.81-6.67 (m, 2H), 6.48 (s, 2H), 5.53-5.44 (m, 2H), 4.71 (m, 1H), 4.58 (m, 1H), 4.50 (m, 1H), 4.29 (m, 1H), 4.18 (m, 1H), 4.13-3.69 (m, 7H), 1.72 (m, 4H), 1.58-1.19 (m, 17H), 0.88 (m, 6H). ³¹P NMR (162 MHz, Acetonitrile-d3) δ 12.68, 12.66. LCMS: MS m/z=680.31 [M+1].

### Example 31. (1-(2,2,2-trifluoroethyl)piperidin-4-yl)methyl ((((2R,3S,4R,5S)-5-(4-aminopyrrolo[2,1-f][1,2,4]triazin-7-yl)-2-cyano3,4-dihydroxytetrahydrofuran-2-yl)methoxy)(phenoxy)phosphoryl)alaninate

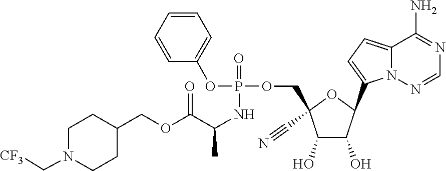 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1CCN(CC(F)(F)F)CC1]

To a mixture of Intermediate 4 (0.5 g, 1.509 mmol), Intermediate 36 (0.905 g, 1.66 mmol), and magnesium chloride (0.206 g, 2.264 mmol) was added tetrahydrofuran (7 mL) followed by the addition of N,N-Diisopropylethylamine (0.657 mL, 3.773 mmol) and the resulting mixture was stirred at 50° C. for 3 h. The reaction mixture was then concentrated under reduced pressure and the residue obtained was diluted with acetonitrile (11 mL) and cooled to 0° C. Concentrated hydrochloric acid (1 mL, 12 mmol) was added and the reaction mixture was stirred at room temperature for 2 h. After 2 h, the reaction mixture was cooled in an ice bath and was neutralized with 5 N aqueous sodium hydroxide solution. The resulting mixture was extracted with ethyl acetate. Organic layer was separated, dried over sodium sulfate, filtered and concentrated. The crude residue was purified via SiO₂ column chromatography (80 g SiO₂ Combiflash HP Gold Column, 100% Dichloromethane-20% Methanol in dichloromethane) to afford the product. ¹H NMR (400 MHz, methanol-d₄) δ 7.79 (d, J=8.8 Hz, 1H), 7.38-7.12 (m, 5H), 6.85 (dd, J=4.5, 1.8 Hz, 1H), 6.74 (dd, J=4.5, 3.2 Hz, 1H), 5.49 (t, J=5.2 Hz, 1H), 4.63 (q, J=5.5 Hz, 1H), 4.55-4.30 (m, 3H), 3.98-3.86 (m, 3H), 3.91-3.76 (m, 2H), 3.07-2.86 (m, 4H), 2.32-2.17 (m, 2H), 1.61 (t, J=12.5 Hz, 4H), 1.26 (ddd, J=7.1, 3.5, 1.1 Hz, 4H). ¹⁹F NMR (377 MHz, methanol-d₄) δ −71.22 (td, J=9.8, 4.6 Hz). ³¹P NMR (162 MHz, methanol-d₄) δ 3.23, 3.18. LCMS: MS m/z=349.86 [M+1]; t_{R}=0.70 min (minor isomer)−0.72 min (major isomer); LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.525 min (minor isomer), 3.56 min (major isomer); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 32. 1-ethyl-3,3-difluoropiperidin-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

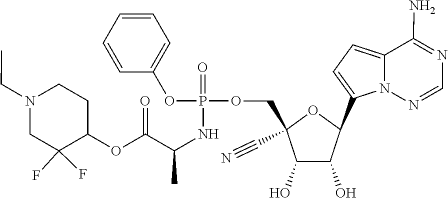 [SMILES: CCN1CCC(OC(=O)[C@H](C)NP(=O)(OC[C@@]2(C#N)O[C@@H](c3ccc4c(N)ncnn34)[C@H](O)[C@@H]2O)Oc2ccccc2)C(F)(F)C1]

To a mixture of Intermediate 4 (50 mg, 0.151 mmol), Intermediate 37 (116 mg, 0.226 mmol), and MgCl₂ (22 mg, 0.226 mmol) in THF (2 mL) was added N,N-diisopropylethylamine (0.1 mL, 0.574 mmol) was added dropwise. The resulting mixture was stirred at 50° C. for 2 h, cooled, purified by prep HPLC (Phenomenex Gemini-NX 10μ C18 110° A 250×30 mm column, 0%-100% acetonitrile/water gradient in 25 min run) to give an acetonide intermediate, which was dissolved in ACN (2 mL) and added c-HCl (0.1 mL). The resulting mixture was stirred at room temperature for 1 h, neutralized with 5 N NaOH, and purified by preparative HPLC (Phenominex Gemini 10 u C18 110 Å 250×21.2 mm column, 20-65% acetonitrile (0.1% TFA)/water (0.1% TFA) gradient). Upon concentration, the residue was dissolved in EtOAc and washed with sat NaHCO₃ solution, concentrated in vacuo, redissolved in DCM and a drop of c-HCl added, which resulted in white precipitation. After concentration, the residue was dissolved in water-acetonitrile, and lyophilized to afford the product. ¹H NMR (400 MHz, Methanol-d4) δ 7.91 (m, 1H), 7.40-7.10 (m, 6H), 6.86 (m, 1H), 5.55-5.47 (m, 1H), 5.30 (m, 1H), 4.57 (m, 1H), 4.49-4.30 (m, 3H), 4.06 (m, 1H), 3.68 (m, 2H), 3.32-3.09 (m, 2H), 2.19 (s, 2H), 1.40-1.23 (m, 8H). ³¹P NMR (162 MHz, Methanol-d₄) δ 3.19, 3.01, 2.97, 2.96. ¹⁹F NMR (376 MHz, Methanol-d₄) δ −77.5. LCMS: MS m/z=666.23 [M+1] as neutral form; t_{R}=0.68 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.35, 3.37, 3.38, 3.41 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 33. (1r,4S)-4-aminocyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

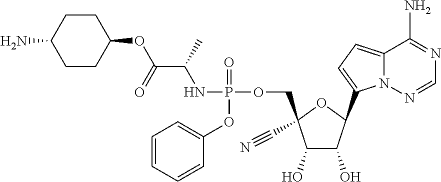 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](N)CC1]

(1r,4S)-4-((tert-butoxycarbonyl)amino)cyclohexyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate. Acetonitrile (4.5 mL) was added to a mixture of Intermediate 4 (300 mg, 0.91 mmol), Intermediate 63 (510 mg, 0.91 mmol), and magnesium chloride (86 mg, 0.91 mmol) at RT. The mixture was heated to 50° C. for 20 min, and N,N-diisopropylethylamine (0.39 mL, 2.26 mmol) was added. After 3.5 h, the reaction mixture was allowed to cool to RT, and the reaction mixture was diluted with ethyl acetate (200 mL) and the resulting mixture was washed with saturated aqueous sodium carbonate solution (200 mL) and brine (200 mL). The organic layer was dried over anhydrous sodium sulfate and concentrated under reduced pressure. The crude residue was subjected to silica gel chromatography eluting with 0-10% methanol in dichloromethane to afford the product. ¹H NMR (400 MHz, chloroform-d₁) δ 7.92 (s, 0.25H), 7.91 (s, 0.75H), 7.35-7.08 (m, 5H), 6.71-6.68 (m, 1H), 6.66-6.62 (m, 1H), 5.92 (br s, 2H), 5.65-5.60 (m, 1H), 5.27-5.22 (m, 1H), 5.10 (d, J=6.7 Hz, 0.25H), 5.00 (d, J=6.6 Hz, 0.75H), 4.69-4.57 (m, 1H), 4.51-4.27 (m, 3H), 4.06-3.92 (m, 1H), 3.86-3.74 (m, 1H), 3.41 (br s, 1H), 2.03-1.84 (m, 4H), 1.76 (br s, 3H), 1.44 (br s, 9H), 1.41-1.29 (m, 8H), 1.24-1.12 (m, 2H). ³¹P NMR (162 MHz, chloroform-d₁) δ −3.15 (s). LCMS: MS m/z=756.11 [M+1].

(1r,4S)-4-aminocyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Concentrated hydrochloric acid solution (12 M, 0.47 mL) was added to a solution of (1r,4S)-4-((tert-butoxycarbonyl)amino)cyclohexyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate (470 mg, 0.62 mmol) in acetonitrile (4.7 mL) at RT. After 1 h, the reaction mixture was diluted with ethyl acetate (20 mL) and neutralized to pH=7 with saturated aqueous sodium carbonate solution. The resulting mixture was concentrated under reduced pressure, and methanol (4 mL) was added to the residue. Ethyl acetate (2 mL) was then added and the resulting solids were removed by filtration. The filtrate was concentrated under reduced pressure and the crude residue was purified by preparatory HPLC (Gemini 5 u C18 100 Å 100×30 mm column, 10-100% acetonitrile/water gradient 0.1% TFA) to afford the product. ¹H NMR (400 MHz, methanol-d₄) δ 8.03 (s, 0.75H), 7.99 (s, 0.25H), 7.42-7.12 (m, 6H), 6.96-6.92 (m, 1H), 5.57-5.51 (m, 1H), 4.74-4.60 (m, 1H), 4.56-4.49 (m, 1H), 4.49-4.34 (m, 3H), 3.96-3.84 (m, 1H), 3.18-3.04 (m, 1H), 2.12-1.99 (m, 4H), 1.57-1.42 (m, 4H), 1.33-1.28 (m, 3H). ³¹P NMR (162 MHz, methanol-d₄) δ 3.36 (s), 3.24 (s). LCMS: MS m/z=616.07 [M+1].

### Example 34

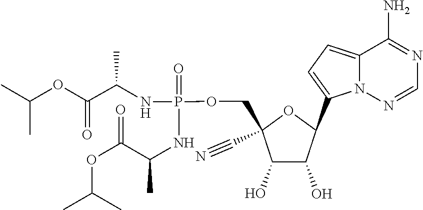 [SMILES: CC(C)OC(=O)[C@H](C)NP(=O)(N[C@@H](C)C(=O)OC(C)C)OC[C@@]1(C#N)O[C@@H](c2ccc3c(N)ncnn23)[C@H](O)[C@@H]1O]

To a mixture of Intermediate 61 (161 mg, 0.36 mmol), Intermediate 4 (100 mg, 0.3 mmol), and MgCl₂ (43 mg, 0.45 mmol) in THF (2 mL) was added N,N-diisopropylethylamine (98 mg, 0.76 mmol) dropwise. The resulting mixture was stirred at about 50° C. for about 2 h, reaction mixture was cooled, diluted with EtOAc, washed with water and brine, the organic solvent was evaporated under vacuum, the residue was then dissolved in acetonitrile (2 mL), cooled in ice bath, and con. HCl was added dropwise. The resulting mixture was stirred at about room temperature for about 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was purified by Prep HPLC to afford the product. ¹H NMR (400 MHz, Methanol-d4) δ 7.83 (s, 1H), 6.87 (d, J=4.5 Hz, 1H), 6.78 (d, J=4.5 Hz, 1H), 5.50 (d, J=4.9 Hz, 1H), 5.00-4.92 (m, 1H), 4.91-4.85 (m, 1H), 4.62 (t, J=5.3 Hz, 1H), 4.50 (d, J=5.7 Hz, 1H), 4.35-4.12 (m, 2H), 3.91-3.72 (m, 2H), 1.34-1.13 (m, 18H). ³¹P NMR (162 MHz, Methanol-d4) δ 13.61. LCMS: MS m/z=598.05 [M+1].

### Example 35. ((2R,3S,4R,5S)-5-(4-aminopyrrolo[2,1-f][1,2,4]triazin-7-yl)-2-cyano-3,4-dihydroxytetrahydrofuran-2-yl)methyl bis-ethyl L-alaninate phosphate

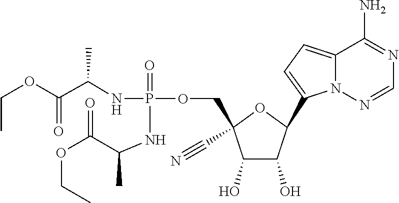 [SMILES: CCOC(=O)[C@H](C)NP(=O)(N[C@@H](C)C(=O)OCC)OC[C@@]1(C#N)O[C@@H](c2ccc3c(N)ncnn23)[C@H](O)[C@@H]1O]

To a mixture of Intermediate 38 (100 mg, 0.3 mmol), Intermediate 4 (151 mg, 0.36 mmol), and MgCl₂ (43 mg, 0.45 mmol) in THF (2 mL) was added N,N-diisopropylethylamine (98 mg, 0.76 mmol) dropwise. The resulting mixture was stirred at 50° C. for 2 h, reaction mixture was cooled, diluted with EtOAc, washed with water and brine, the organic solvent was evaporated under vacuum, the residue was then dissolved in acetonitrile (2 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue purified by Prep HPLC to afford the product. ¹H NMR (400 MHz, Methanol-d4) δ 7.82 (s, 1H), 6.85 (d, J=4.5 Hz, 1H), 6.77 (d, J=4.5 Hz, 1H), 5.50 (d, J=4.9 Hz, 1H), 4.63 (t, J=5.3 Hz, 1H), 4.50 (d, J=5.7 Hz, 1H), 4.30 (dd, J=11.1, 7.0 Hz, 1H), 4.23-3.99 (m, 5H), 3.86 (ddq, J=19.6, 9.4, 7.1 Hz, 2H), 1.35-1.13 (m, 12H). ³¹P NMR (162 MHz, Methanol-d4) δ 13.61. LCMS: MS m/z=570.10 [M+1], t_{R}=0.99 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.19 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 36. (2R,3S,4R,5S)-5-(4-aminopyrrolo[2,1-f][1,2,4]triazin-7-yl)-2-cyano-3,4-dihydroxytetrahydrofuran-2-yl)methyl bis-cyclobutylmethyl L-alaninate phosphate

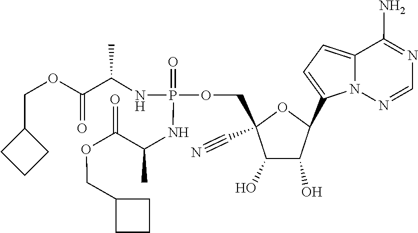 [SMILES: C[C@H](NP(=O)(N[C@@H](C)C(=O)OCC1CCC1)OC[C@@]1(C#N)O[C@@H](c2ccc3c(N)ncnn23)[C@H](O)[C@@H]1O)C(=O)OCC1CCC1]

To a mixture of Intermediate 62 (132 mg, 0.27 mmol), Intermediate 4 (80 mg, 0.24 mmol), and MgCl₂ (34 mg, 0.36 mmol) in THF (2 mL) was added N,N-diisopropylethylamine (78 mg, 0.6 mmol) dropwise. The resulting mixture was stirred at 50° C. for 2 h, reaction mixture was cooled, diluted with EtOAc, washed with water and brine, the organic solvent was evaporated under vacuum, the residue was then dissolved in acetonitrile (2 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was purified by Prep HPLC to afford the product. ¹H NMR (400 MHz, Methanol-d4) δ 7.82 (s, 1H), 6.85 (d, J=4.5 Hz, 1H), 6.77 (d, J=4.5 Hz, 1H), 5.50 (d, J=4.9 Hz, 1H), 4.61 (t, J=5.3 Hz, 1H), 4.49 (d, J=5.7 Hz, 1H), 4.30 (dd, J=11.1, 7.1 Hz, 1H), 4.20 (dd, J=11.1, 5.7 Hz, 1H), 4.14-3.98 (m, 3H), 3.98-3.80 (m, 3H), 2.60 (dp, J=22.0, 7.4 Hz, 2H), 2.10-1.95 (m, 4H), 1.94-1.64 (m, 8H), 1.39-1.17 (m, 6H). ³¹P NMR (162 MHz, Methanol-d4) δ 13.54. LCMS: MS m/z=650.12 [M+1].

### Example 37. benzyl ((((2R,3S,4R,5S)-5-(4-aminopyrrolo[2,1-f][1,2,4]triazin-7-yl)-2-cyano-3,4-dihydroxytetrahydrofuran-2-yl)methoxy) (phenoxy)phosphoryl)-L-alaninate

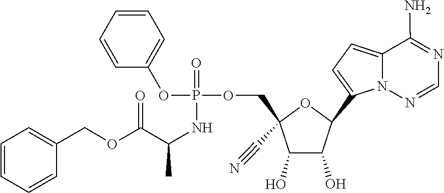 [SMILES: C[C@H](NP(=O)(OC[C@@]1(C#N)O[C@@H](c2ccc3c(N)ncnn23)[C@H](O)[C@@H]1O)Oc1ccccc1)C(=O)OCc1ccccc1]

Intermediate 4 (83 mg, 0.25 mmol) was mixed with Intermediate 39 (126 mg, 0.275 mmol) and dissolved in 2 mL of anhydrous tetrahydrofuran. Magnesium chloride (71 mg, 0.75 mmol) was added in one portion. DIPEA (87 μL, 0.5 mmol) was then added, and the reaction was stirred at 60° C. for 16 h.

More Intermediate 39 (30 mg) and DIPEA (52 μL) were added. The reaction mixture was stirred at 60° C. for 6 h. The reaction mixture was then cooled to RT, diluted with ethyl acetate (10 mL) and washed with water (5×10 mL) followed with brine (5 mL). The organic extract was dried over anhydrous sodium sulfate and concentrated under reduced pressure. The resulted material was dissolved in 5 mL of MeCN and stirred in an ice bath. Concentrated HCl (aq) (300 μL) was added dropwise, and reaction was stirred in an ice bath for 2 h. The reaction mixture was diluted with ethyl acetate (10 mL) and washed with saturated aqueous sodium bicarbonate solution and followed with brine. Organic extract was dried over anhydrous sodium sulfate and concentrated under reduced pressure. The crude residue was purified via SiO₂ column chromatography (4 g SiO₂ Combiflash HP Gold Column, 0-10% methanol in dichloromethane). Fractions containing the desired product were combined and concentrated under reduced pressure as oil which was then dissolved in MeCN and water and freeze-dried to afford the product. ¹H NMR (400 MHz, methanol-d₄) δ 7.77 (m, 1H), 7.35-7.08 (m, 10H), 6.83 (m, 1H), 6.71 (m, 1H), 5.52-5.48 (m, 1H), 5.14-4.93 (m, 2H), 4.61 (m, 1H), 4.53-4.27 (m, 3H), 4.01-3.87 (m, 1H), 1.26 (m, 3H). ³¹P NMR (162 MHz, methanol-d₄) δ 3.22, 3.19. LCMS: MS m/z=609.1 [M+1]; 607.4 [M−1], t_{R}=1.19 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.78 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=4.626 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 38

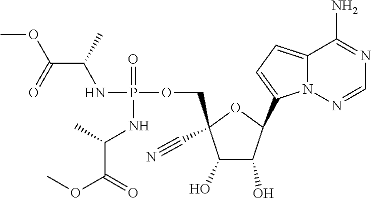 [SMILES: COC(=O)[C@H](C)NP(=O)(N[C@@H](C)C(=O)OC)OC[C@@]1(C#N)O[C@@H](c2ccc3c(N)ncnn23)[C@H](O)[C@@H]1O]

Acetonitrile (1 mL) was added to a mixture of Intermediate 4 (150 mg, 0.453 mmol), Intermediate 40 (176 mg, 0.453 mmol), and magnesium chloride (43 mg, 0.453 mmol) at RT. The mixture was heated to 50° C. for 10 min, and N,N-diisopropylethylamine (0.197 mL, 1.13 mmol) was added. After 2 h, the reaction mixture was allowed to cool to RT, and concentrated aqueous hydrochloric acid solution (0.25 mL) was added dropwise. After 1 h, the reaction mixture was diluted with ethyl acetate (20 mL) and the resulting mixture was washed with saturated aqueous sodium carbonate solution (20 mL) and brine (20 mL). The organic layer was dried over anhydrous sodium sulfate and concentrated under reduced pressure. The crude residue was subjected to silica gel chromatography eluting with 0-100% ethyl acetate in hexanes to afford the product. ¹H NMR (400 MHz, methanol-d₄) δ 7.82 (s, 1H), 6.86 (d, J=4.5 Hz, 1H), 6.77 (d, J=4.5 Hz, 1H), 5.50 (d, J=4.9 Hz, 1H), 4.64 (dd, J=5.6, 5.0 Hz, 1H), 4.51 (d, J=5.7 Hz, 1H), 4.30 (dd, J=11.1, 6.9 Hz, 1H), 4.19 (dd, J=11.1, 5.6 Hz, 1H), 3.86 (ddd, J=14.7, 9.4, 7.2 Hz, 2H), 3.69 (s, 3H), 3.64 (s, 3H), 1.30 (dd, J=7.2, 1.0 Hz, 3H), 1.25 (dd, J=7.2, 0.8 Hz, 3H). ³¹P NMR (162 MHz, methanol-d₄) δ 13.58 (s). LCMS: MS m/z=542.08 [M+1], t_{R}=0.88 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=1.87 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=3.052 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 39. methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

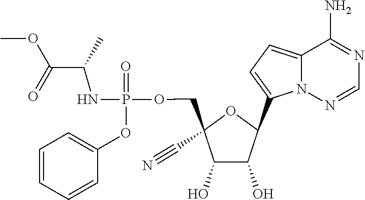 [SMILES: COC(=O)[C@H](C)NP(=O)(OC[C@@]1(C#N)O[C@@H](c2ccc3c(N)ncnn23)[C@H](O)[C@@H]1O)Oc1ccccc1]

Acetonitrile (5 mL) was added to a mixture of Intermediate 4 (348 mg, 1.05 mmol), Intermediate 41 (399 mg, 1.05 mmol), and magnesium chloride (100 mg, 1.05 mmol) at RT. The mixture was heated to 50° C. for 10 min, and N,N-diisopropylethylamine (0.475 mL, 2.63 mmol) was added. After 2.5 h, the reaction mixture was allowed to cool to RT, and concentrated aqueous hydrochloric acid solution (0.5 mL) was added dropwise. After 1 h, the reaction mixture was diluted with ethyl acetate (100 mL) and the resulting mixture was washed with saturated aqueous sodium carbonate solution (100 mL) and brine (100 mL). The organic layer was dried over anhydrous sodium sulfate and concentrated under reduced pressure. The crude residue was subjected to silica gel chromatography eluting with 0-100% ethyl acetate in hexanes to afford the product. ¹H NMR (400 MHz, methanol-d₄) δ 7.80 (s, 0.65H), 7.78 (s, 0.35H), 7.37-7.25 (m, 2H), 7.25-7.12 (m, 3H), 6.87-6.82 (m, 1H), 6.75-6.71 (m, 1H), 5.52-5.47 (m, 1H), 4.66-4.60 (m, 1H), 4.55-4.29 (m, 3H), 3.95-3.80 (m, 1H), 3.64 (s, 1H), 3.60 (s, 2H), 1.27-1.22 (m, 3H). ³¹P NMR (162 MHz, methanol-d₄) δ 3.24 (s). LCMS: MS m/z=533.13 [M+1], t_{R}=1.02 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.28 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=3.712 min (minor isomer), 3.775 min (major isomer); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 40. isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(2-(methylthio) ethoxy)phosphoryl)-L-alaninate

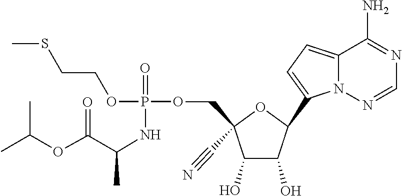 [SMILES: CSCCOP(=O)(N[C@@H](C)C(=O)OC(C)C)OC[C@@]1(C#N)O[C@@H](c2ccc3c(N)ncnn23)[C@H](O)[C@@H]1O]

Intermediate 4 (50 mg, 0.15 mmol) and Intermediate 70 (67 mg, 0.165 mmol) were mixed and dissolved in 1.5 mL of anhydrous tetrahydrofuran. Magnesium chloride (43 mg, 0.45 mmol) was in one portion. DIPEA (65 μL, 0.75 mmol) was added, and the reaction was stirred at RT for 36 h. The reaction was diluted with ethyl acetate (15 mL) and washed with water (5×10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-3% methanol/dichloromethane). Fractions containing the desired product were combined and concentrated under reduced pressure. The residue was dissolved in MeCN (5 mL) and stirred in an ice bath. Concentrate aqueous hydrochloric acid (300 μL) was added dropwise. The reaction mixture was stirred in an ice bath for 2 h. The reaction mixture was diluted with ethyl acetate (15 mL) and added saturated aqueous sodium bicarbonate solution (10 mL). The mixture was stirred for 10 min. The organic extract was collected and washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-5% methanol/dichloromethane). Fractions containing the desired product were combined and concentrated under reduced pressure. The residue was dissolved in MeCN and water and freeze-dried to give the product. ¹H NMR (400 MHz, methanol-d₄) δ 7.81 (m, 1H), 6.85 (m, 1H), 6.76 (m, 1H), 5.50 (m, 1H), 4.92 (m, 1H), 4.64 (m, 1H), 4.50 (m, 1H), 4.40-4.21 (m, 2H), 4.17 (m, 1H), 4.09 (m, 1H), 3.85-3.72 (m, 1H), 2.72 (m, 2H), 2.09 (m, 3H), 1.29 (m, 3H), 1.25-1.21 (m, 3H), 1.17 (m, 3H). ³¹P NMR (162 MHz, methanol-d₄) δ 7.74, 7.82. LCMS: MS m/z=559.0 [M+1]; 557.2 [M−1], t_{R}=1.04 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=2.36 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.976, 4.022 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 41. isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(2-methoxy ethoxy)phosphoryl)-L-alaninate

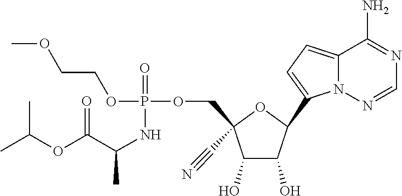 [SMILES: COCCOP(=O)(N[C@@H](C)C(=O)OC(C)C)OC[C@@]1(C#N)O[C@@H](c2ccc3c(N)ncnn23)[C@H](O)[C@@H]1O]

Intermediate 4 (50 mg, 0.15 mmol) and Intermediate 71 (64 mg, 0.165 mmol) were mixed and dissolved in 1.5 mL of anhydrous tetrahydrofuran. Magnesium chloride (43 mg, 0.45 mmol) was added in one portion. DIPEA (65 μL, 0.375 mmol) was added, and the reaction was stirred at RT for 20 h.

The reaction mixture was diluted with ethyl acetate (15 mL) and washed with water (5×10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-3% methanol/dichloromethane). Fractions containing the desired product were combined and concentrated under reduced pressure. The resulting material was dissolved in MeCN (5 mL) and stirred in an ice bath. Concentrate aqueous hydrochloric acid (300 μL) was added dropwise. The reaction mixture was stirred in an ice bath for 2 h. The reaction was diluted with ethyl acetate (15 mL) and saturated aqueous sodium bicarbonate solution was added. The mixture was stirred for 10 min. The organic extract was washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-5% methanol/dichloromethane). Fractions containing the desired product were combined and concentrated under reduced pressure. The residue was dissolved in MeCN and water and freeze-dried to give the product. ¹H NMR (400 MHz, methanol-d₄) δ 7.82 (s, 1H), 6.89-6.83 (m, 1H), 6.76 (m, 1H), 5.50 (m, 1H), 5.01-4.84 (m, 1H), 4.63 (m, 1H), 4.50 (m, 1H), 4.35 (m, 1H), 4.30-4.19 (m, 1H), 4.19-4.13 (m, 2H), 3.77 (m, 1H), 3.63-3.51 (m, 2H), 3.35 (m, 3H), 1.28 (m, 3H), 1.17 (m, 6H). ³¹P NMR (162 MHz, methanol-d₄) δ 7.98, 8.04. LCMS: MS m/z=543.1 [M+1]; 541.2 [M−1], t_{R}=0.96 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=2.18 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.599, 3.619 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 42. isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(2-(methylsulfonyl)ethoxy)phosphoryl)-L-alaninate

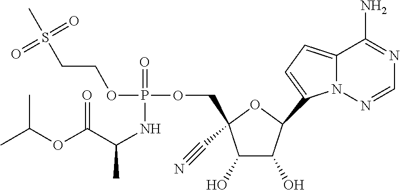 [SMILES: CC(C)OC(=O)[C@H](C)NP(=O)(OCCS(C)(=O)=O)OC[C@@]1(C#N)O[C@@H](c2ccc3c(N)ncnn23)[C@H](O)[C@@H]1O]

Intermediate 4 (66 mg, 0.2 mmol) and Intermediate 72 (100 mg, 0.22 mmol) were mixed and dissolved in 2 mL of anhydrous tetrahydrofuran. Magnesium chloride (57 mg, 0.6 mmol) was added in one portion. DIPEA (87 μL, 0.5 mmol) was added and the reaction was stirred at 35° C. for 16 h. The reaction was diluted with ethyl acetate (15 mL) and washed with water (5×10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-100% B/hexanes (B=3% MeOH in ethyl acetate)). Fractions containing the desired product were combined and concentrated under reduced pressure.

The resulting material was dissolved in MeCN (5 mL) and stirred in an ice bath. Concentrate aqueous hydrochloric acid (250 μL) was added dropwise. The reaction was stirred in an ice bath for 2 h. The reaction was diluted with ethyl acetate (15 mL) and added saturated aqueous sodium bicarbonate solution (10 mL). The mixture was stirred for 10 min. The organic extract was washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-10% methanol/dichloromethane). Fractions containing the desired product were combined and concentrated under reduced pressure. The residue was dissolved in MeCN and water and freeze-dried to give the product. ¹H NMR (400 MHz, methanol-d₄) δ 7.82 (m, 1H), 6.86 (m, 1H), 6.77 (m, 1H), 5.50 (m, 1H), 5.03-4.85 (m, 1H), 4.64 (m, 1H), 4.54-4.44 (m, 2H), 4.43-4.21 (m, 2H), 3.80 (m, 1H), 3.57-3.36 (m, 2H), 2.97 (m, 3H), 1.30 (m, 3H), 1.26-1.14 (m, 6H). ³¹P NMR (162 MHz, methanol-d₄) δ 7.79, 7.92. LCMS: MS m/z=591.1 [M+1], t_{R}=0.92 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.07 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.435 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 43. Isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(4-(dimethylcarbamoyl)phenoxy)phosphoryl)-L-alaninate

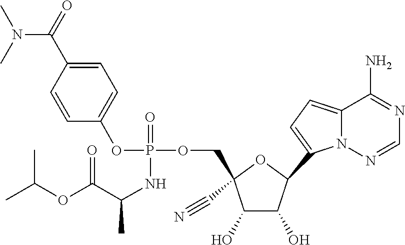 [SMILES: CC(C)OC(=O)[C@H](C)NP(=O)(OC[C@@]1(C#N)O[C@@H](c2ccc3c(N)ncnn23)[C@H](O)[C@@H]1O)Oc1ccc(C(=O)N(C)C)cc1]

To a mixture of Intermediate 4 (70 mg, 0.211 mmol), Intermediate 43 (160 mg, 0.317 mmol), and MgCl₂ (30 mg, 0.317 mmol) in THF (3 mL) was added N,N-diisopropylethylamine (0.1 mL, 0.528 mmol) dropwise at room temperature. The resulting mixture was stirred at 50° C. for 2 h, and purified by preparative HPLC (Phenomenex Gemini-NX 10μ C18 110° A 250×30 mm column, 0%-100% acetonitrile/water gradient in 25 min run) to give an acetonide intermediate, which was dissolved in acetonitrile (2 mL) and c-HCl (0.2 mL) was added. The mixture was stirred for 2 h, aq. NaHCO₃ (2 mL) added under ice bath, and purified by preparative HPLC (Phenomenex Gemini-NX 10μ C18 110° A 250×30 mm column, 0%-100% acetonitrile/water gradient in 25 min run) to afford the product. ¹H NMR (400 MHz, Methanol-d4) δ 7.80 (m, 1H), 7.40 (m, 1H), 7.37-7.28 (m, 2H), 7.23 (m, 1H), 6.86 (m, 1H), 6.74 (m, 1H), 5.50 (m, 1H), 5.00-4.81 (m, 1H), 4.61 (m, 1H), 4.54-4.38 (m, 2H), 4.35 (m, 1H), 3.92-3.79 (m, 1H), 3.07 (d, J=3.4 Hz, 3H), 2.95 (m, 3H), 1.28 (m, 3H), 1.22-1.12 (m, 6H). ³¹P NMR (162 MHz, Methanol-d4) δ 3.18. LCMS: MS m/z=632.32 [M+1]; t_{R}=0.67 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.84 min (18%), 3.85 (81%); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min

### Example 44. oxetan-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

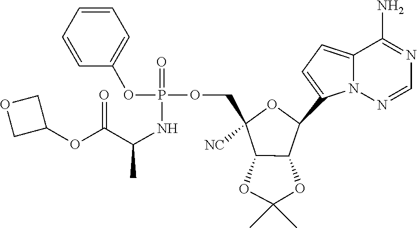 [SMILES: C[C@H](NP(=O)(OC[C@@]1(C#N)O[C@@H](c2ccc3c(N)ncnn23)[C@@H]2OC(C)(C)O[C@@H]21)Oc1ccccc1)C(=O)OC1COC1]

oxetan-3-yl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate. To a mixture of Intermediate 44 (133 mg, 0.31 mmol), Intermediate 4 (130 mg, 0.39 mmol), and MgCl₂ (45 mg, 0.47 mmol) in THF (5 mL) was added N,N-diisopropylethylamine (127 mg, 0.98 mmol) dropwise. The resulting mixture was stirred at 50° C. for 2 h, reaction mixture was cooled, diluted with EtOAc, washed with water and brine, the organic solvent was evaporated under vacuum, the residue was purified with silica gel column chromatography eluting with 0-100% MeOH in DCM to afford the product. LCMS: MS m/z=615.18 [M+1], t_{R}=1.18 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.40 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

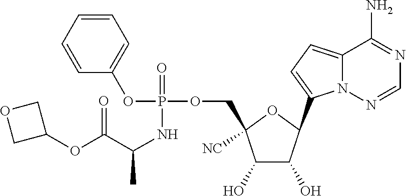 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1COC1]

oxetan-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Dissolved oxetan-3-yl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate (500 mg, 0.81 mmol) in 10 mL ACN, mixed 20 mL of TFA with 10 mL water, then added the TFA solution to above reaction mixture, stirred at RT for 30 mins, quenched with aq. NaHCO₃ solution, extracted with EtOAc, evaporated organic solvent, purified with Prep HPLC to afford the product. ¹H NMR (400 MHz, Methanol-d4) δ 7.79 (d, J=9.7 Hz, 1H), 7.38-7.11 (m, 5H), 6.85 (d, J=4.5 Hz, 1H), 6.73 (dd, J=5.6, 4.5 Hz, 1H), 5.50 (t, J=4.5 Hz, 1H), 5.32 (dtt, J=23.9, 6.3, 5.1 Hz, 1H), 4.82-4.73 (m, 2H), 4.63 (td, J=5.3, 4.1 Hz, 1H), 4.60-4.44 (m, 4H), 4.44-4.26 (m, 2H), 4.01-3.85 (m, 1H), 1.29 (dt, J=7.2, 1.3 Hz, 3H). ³¹P NMR (162 MHz, Methanol-d4) δ 3.3, 3.29. LCMS: MS m/z=575.11 [M+1], t_{R}=0.98 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.63 and 3.70 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 45. propyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

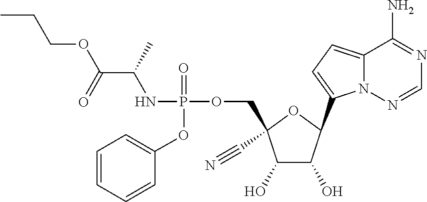 [SMILES: CCCOC(=O)[C@H](C)NP(=O)(OC[C@@]1(C#N)O[C@@H](c2ccc3c(N)ncnn23)[C@H](O)[C@@H]1O)Oc1ccccc1]

N,N-Diisopropylethylamine (0.33 mL, 1.89 mmol) and magnesium chloride (107.8 mg, 1.13 mmol) were added to a mixture of Intermediate 4 (250.0 mg, 0.76 mmol) and Intermediate 45 (462.16 mg, 1.13 mmol) in tetrahydrofuran (7.5 mL) at RT. The mixture was heated to 55° C. After 2 h, the reaction mixture was allowed to cool to RT, diluted with ethyl acetate (30 mL) and the resulting mixture was washed with water (5×20 mL) and brine (20 mL). The organic layer was dried over anhydrous sodium sulfate and concentrated under reduced pressure. Concentrated aqueous hydrochloric acid solution (0.53 mL) was added dropwise to the crude residue in acetonitrile (7.5 mL) at 0° C. The mixture was warmed to RT. After 2 h, the reaction mixture was diluted with ethyl acetate (100 mL) and the resulting mixture was washed with saturated aqueous sodium carbonate solution (75 mL) and brine (75 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Methanol-d₄) δ 7.80 (d, J=7.2 Hz, 1H), 7.37-7.27 (m, 2H), 7.26-7.13 (m, 3H), 6.85 (dd, J=4.5, 2.9 Hz, 1H), 6.74 (dd, J=4.6, 2.1 Hz, 1H), 5.50 (t, J=5.3 Hz, 1H), 4.63 (q, J=5.3 Hz, 1H), 4.54-4.31 (m, 3H), 4.07-3.82 (m, 3H), 1.68-1.49 (m, 2H), 1.31-1.26 (m, 3H), 0.90 (dt, J=9.9, 7.4 Hz, 3H). ³¹P NMR (162 MHz, Methanol-d₄) δ 3.27. LCMS: MS m/z=561.20 [M+1], t_{R}=0.78 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.70 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

Resolution of the Sp and Rp diastereomers. The product was purified via chiral SFC (Chiralpak AD-H, 5 um, 21×250 mm, Isopropyl alcohol 30%) to afford the diastereomers:

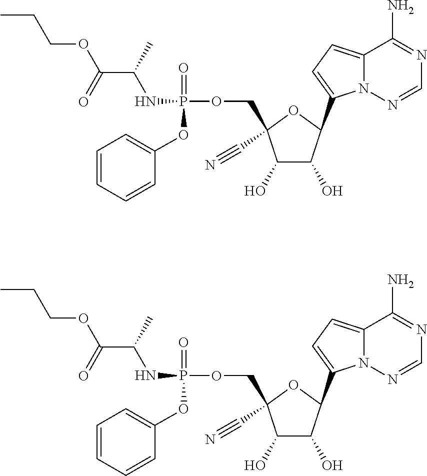 [SMILES: CCCOC(=O)[C@H](C)N[P@](=O)(OC[C@@]1(C#N)O[C@@H](c2ccc3c(N)ncnn23)[C@H](O)[C@@H]1O)Oc1ccccc1 ; CCCOC(=O)[C@H](C)N[P@@](=O)(OC[C@@]1(C#N)O[C@@H](c2ccc3c(N)ncnn23)[C@H](O)[C@@H]1O)Oc1ccccc1]

### Example 46. First Eluting Diastereomer of Example 45

¹H NMR (400 MHz, Methanol-d₄) δ 7.79 (s, 1H), 7.33-7.26 (m, 2H), 7.20-7.12 (m, 3H), 6.85 (d, J=4.5 Hz, 1H), 6.73 (d, J=4.5 Hz, 1H), 5.51 (d, J=5.0 Hz, 1H), 4.68-4.60 (m, 1H), 4.53 (d, J=5.6 Hz, 1H), 4.48 (dd, J=10.9, 6.0 Hz, 1H), 4.36 (dd, J=10.9, 5.1 Hz, 1H), 4.06-3.95 (m, 2H), 3.88 (dq, J=9.4, 7.1 Hz, 1H), 1.62 (h, J=7.3 Hz, 2H), 1.26 (dd, J=7.1, 1.3 Hz, 3H), 0.91 (t, J=7.5 Hz, 3H). ³¹P NMR (162 MHz, Methanol-d₄) δ 3.26. LCMS: MS m/z=561.21 [M+1], t_{R}=0.76 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.63 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

### Example 47. Second Eluting Diastereomer of Example 45

¹H NMR (400 MHz, Methanol-d₄) δ 7.80 (s, 1H), 7.37-7.29 (m, 2H), 7.26-7.14 (m, 3H), 6.84 (d, J=4.5 Hz, 1H), 6.74 (d, J=4.5 Hz, 1H), 5.50 (d, J=5.0 Hz, 1H), 4.62 (dd, J=5.6, 5.1 Hz, 1H), 4.47 (d, J=5.6 Hz, 1H), 4.42 (dd, J=10.9, 6.3 Hz, 1H), 4.34 (dd, J=10.9, 5.5 Hz, 1H), 4.02-3.85 (m, 3H), 1.58 (dtd, J=14.0, 7.4, 6.6 Hz, 2H), 1.27 (dd, J=7.2, 1.1 Hz, 3H), 0.88 (t, J=7.5 Hz, 3H). ³¹P NMR (162 MHz, Methanol-d₄) δ 3.27. LCMS: MS m/z=561.26 [M+1], t_{R}=0.77 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.74 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

### Example 48. oxetan-3-ylmethyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate

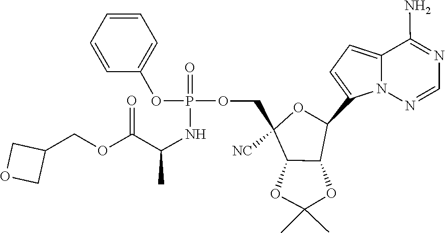 [SMILES: C[C@H](NP(=O)(OC[C@@]1(C#N)O[C@@H](c2ccc3c(N)ncnn23)[C@@H]2OC(C)(C)O[C@@H]21)Oc1ccccc1)C(=O)OCC1COC1]

To a mixture of Intermediate 46 (350 mg, 1.06 mmol), Intermediate 4 (507 mg, 1.16 mmol), and MgCl₂ (130 mg, 1.37 mmol) in THF (10 mL) was added N,N-diisopropylethylamine (341 mg, 2.64 mmol) dropwise. The resulting mixture was stirred at 50° C. for 2 h, reaction mixture was cooled, diluted with EtOAc, washed with water and brine, the organic solvent was evaporated under vacuum, the residue was purified with silica gel column chromatography eluting with 0-100% MeOH in DCM to afford the product. LCMS: MS m/z=629.10 [M+1], t_{R}=1.21 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.51 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 49. oxetan-3-ylmethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

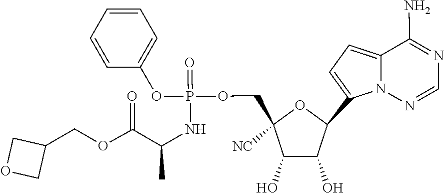 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1COC1]

Dissolved Example 48 (385 mg, 0.61 mmol) in 12 mL ACN, mixed 17 mL of TFA with 12 mL water, then added the TFA solution to above reaction mixture, stirred at RT for 30 mins, quenched with aq. NaHCO₃ solution, extracted with EtOAc, evaporated organic solvent, purified with Prep HPLC to afford the product. ¹H NMR (400 MHz, Methanol-d4) δ 7.80 (d, J=8.5 Hz, 1H), 7.31 (dt, J=16.0, 7.8 Hz, 2H), 7.25-7.10 (m, 3H), 6.86 (dd, J=4.6, 2.8 Hz, 1H), 6.74 (t, J=4.3 Hz, 1H), 5.49 (t, J=4.9 Hz, 1H), 4.72 (dddd, J=11.0, 7.9, 6.3, 3.6 Hz, 2H), 4.67-4.57 (m, 1H), 4.55-4.29 (m, 5H), 4.29-4.23 (m, 1H), 4.24-4.09 (m, 1H), 3.91 (m, 1H), 3.28-3.10 (m, 1H), 1.27 (ddd, J=7.2, 2.7, 1.2 Hz, 3H). ³¹P NMR (162 MHz, Methanol-d4) δ 3.22, 3.15. LCMS: MS m/z=589.15 [M+1], t_{R}=1.01 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.66 and 3.72 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 50. cyclobutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate single diastereomer

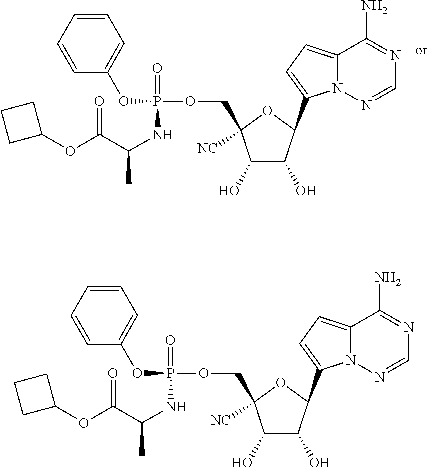 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCC1]

To a mixture of Intermediate 48 (330 mg, 0.79 mmol), Intermediate 4 (260 mg, 0.79 mmol), and MgCl₂ (97 mg, 1.02 mmol) in THF (10 mL) was added N,N-diisopropylethylamine (254 mg, 1.96 mmol) dropwise. The resulting mixture was stirred at 50° C. for 2 h, reaction mixture was cooled, diluted with EtOAc, washed with water and brine, the organic solvent was evaporated under vacuum, the residue was purified with silica gel column chromatography eluting with 0-100% MeOH in DCM to afford acetonide intermediate, which is then dissolved in acetonitrile (10 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was dissolved in DCM and purified by silica gel column chromatography eluting with 0-100% MeOH in DCM to afford the product. ¹H NMR (400 MHz, Methanol-d4) δ 7.78 (s, 1H), 7.31 (dd, J=8.7, 7.1 Hz, 2H), 7.22 (dt, J=8.6, 1.3 Hz, 2H), 7.20-7.08 (m, 1H), 6.84 (d, J=4.5 Hz, 1H), 6.72 (d, J=4.5 Hz, 1H), 5.51 (dt, J=5.0, 1.4 Hz, 1H), 4.82-4.80 (m, 1H), 4.63 (t, J=5.3 Hz, 1H), 4.53-4.38 (m, 2H), 4.35 (ddd, J=10.3, 5.0, 1.4 Hz, 1H), 3.86 (dq, J=9.7, 7.1 Hz, 1H), 2.32-2.09 (m, 2H), 2.04-1.89 (m, 2H), 1.79-1.64 (m, 1H), 1.64-1.46 (m, 1H), 1.29-1.18 (m, 3H). ³¹P NMR (162 MHz, Methanol-d4) δ 3.25. LCMS: MS m/z=573.11 [M+1], t_{R}=1.12 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=4.395 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 51. cyclobutyl (((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate single diastereomer

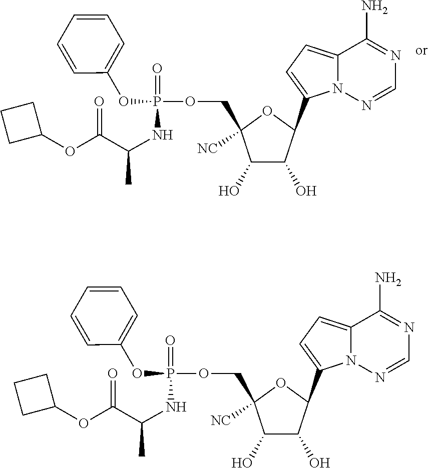 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCC1]

To a mixture of Intermediate 49 (355 mg, 0.85 mmol), Intermediate 4 (280 mg, 0.85 mmol), and MgCl₂ (105 mg, 1.1 mmol) in THF (10 mL) was added N,N-diisopropylethylamine (273 mg, 2.1 mmol) dropwise. The resulting mixture was stirred at 50° C. for 2 h, reaction mixture was cooled, diluted with EtOAc, washed with water and brine, the organic solvent was evaporated under vacuum, the residue was purified with silica gel column chromatography eluting with 0-100% MeOH in DCM to afford acetonide intermediate, which is then dissolved in acetonitrile (10 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was dissolved in DCM and purified by silica gel column chromatography eluting with 0-100% MeOH in DCM to afford the product. ¹H NMR (400 MHz, Methanol-d4) δ 7.78 (s, 1H), 7.28 (dd, J=8.8, 7.0 Hz, 2H), 7.20-7.08 (m, 3H), 6.85 (d, J=4.5 Hz, 1H), 6.73 (d, J=4.6 Hz, 1H), 5.51 (d, J=5.0 Hz, 1H), 4.97-4.86 (m, 1H), 4.63 (t, J=5.3 Hz, 1H), 4.52 (d, J=5.6 Hz, 1H), 4.47 (dd, J=10.9, 5.9 Hz, 1H), 4.35 (dd, J=10.9, 5.1 Hz, 1H), 3.84 (dq, J=9.2, 7.1 Hz, 1H), 2.34-2.19 (m, 2H), 2.13-1.91 (m, 2H), 1.84-1.69 (m, 1H), 1.69-1.52 (m, 1H), 1.25 (dd, J=7.2, 1.2 Hz, 3H). ³¹P NMR (162 MHz, Methanol-d4) δ 3.21. LCMS: MS m/z=573.10 [M+1], t_{R}=1.15 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=4.364 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 52. methyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

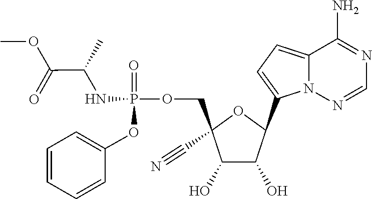 [SMILES: COC(=O)[C@H](C)N[P@](=O)(OC[C@@]1(C#N)O[C@@H](c2ccc3c(N)ncnn23)[C@H](O)[C@@H]1O)Oc1ccccc1]

Method 1. N,N-Diisopropylethylamine (0.12 mL, 0.68 mmol) and magnesium chloride (38.8 mg, 0.41 mmol) were added to a mixture of Intermediate 4 (100.0 mg, 0.30 mmol) and Intermediate 50 (141.2 mg, 0.33 mmol) in tetrahydrofuran (3 mL) at RT. The mixture was heated to 50° C. After 1 h, the reaction mixture was allowed to cool to RT, diluted with ethyl acetate (25 mL) and the resulting mixture was washed with water (5×10 mL) and brine (10 mL). The organic layer was dried over anhydrous sodium sulfate and concentrated under reduced pressure. Concentrated aqueous hydrochloric acid solution (0.2 mL) was added dropwise to the crude residue in acetonitrile (3 mL) at 0° C. The mixture was warmed to RT. After 3 h, the reaction mixture was diluted with ethyl acetate (25 mL) and the resulting mixture was washed with saturated aqueous sodium carbonate solution (20 mL) and brine (20 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Acetonitrile-d₃) δ 7.87 (s, 1H), 7.40-7.30 (m, 2H), 7.27-7.14 (m, 3H), 6.73 (s, 2H), 6.20 (s, 2H), 5.46 (d, J=5.0 Hz, 1H), 4.63-4.51 (m, 1H), 4.51-4.40 (m, 1H), 4.35 (dd, J=11.1, 6.6 Hz, 2H), 4.28 (dd, J=11.1, 6.4 Hz, 2H), 4.00-3.83 (m, 2H), 3.59 (s, 3H), 1.26 (dd, J=7.1, 1.0 Hz, 3H). ³¹P NMR (162 MHz, Acetonitrile-d₃) δ 2.64. LCMS: MS m/z=533.15 [M+1], t_{R}=0.65 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.03 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

Method 2. Intermediate 4 (150 mg, 0.5 mmol) and Intermediate 50 (234 mg, 0.55 mmol) were mixed and dissolved in 4 mL of anhydrous THF. Magnesium chloride (143 mg, 1.5 mmol) was added in one portion. DIPEA (218 uL, 1.25 mmol) was added, and the reaction was stirred at 50° C. for 4 hrs.

Reaction was diluted with EtOAc (20 mL) and washed with water (5×15 mL) and then with brine (5 mL). Organic extract was dried over anhydrous sodium sulfate and concentrated under reduced pressure. Residue was dissolved in MeCN (10 mL) and stirred in an ice bath. Concentrate aqueous hydrochloric acid (500 uL) was added dropwise. Reaction was stirred in an ice bath for 4 hrs. Reaction was diluted with EtOAc (30 mL) and added saturated aqueous sodium bicarbonate solution (30 mL). Mixture was stirred for 10 mins. Organic extract was collected and washed with brine (10 mL). Organic extract was dried over anhydrous sodium sulfate and concentrated under reduced pressure. The crude residue was purified via SiO₂ column chromatography (12 g SiO₂ Combiflash HP Gold Column, 0-10% methanol/DCM). Fractions having the desired product were combined and concentrated under reduced pressure. Residue was dissolved in MeCN and water and freeze-dried to give the product. ¹H NMR (400 MHz, methanol-d₄) δ 7.79 (s, 1H), 7.37-7.27 (m, 2H), 7.22 (m, 2H), 7.16 (m, 1H), 6.83 (d, J=4.5 Hz, 1H), 6.73 (d, J=4.5 Hz, 1H), 5.51 (d, J=5.1 Hz, 1H), 4.63 (t, J=5.3 Hz, 1H), 4.48 (d, J=5.6 Hz, 1H), 4.45-4.30 (m, 2H), 3.90 (m, 1H), 3.59 (s, 3H), 1.25 (d, J=7.1 Hz, 3H). ³¹P NMR (162 MHz, methanol-d₄) δ 3.24. LCMS: MS m/z=533.0 [M+1], 531.0 [M−1], t_{R}=1.31 min; LC system: Thermo Dionex Ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.29 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.791 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 53. isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

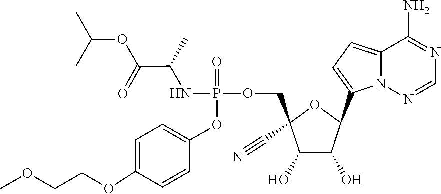 [SMILES: COCCOc1ccc(OP(=O)(N[C@@H](C)C(=O)OC(C)C)OC[C@@]2(C#N)O[C@@H](c3ccc4c(N)ncnn34)[C@H](O)[C@@H]2O)cc1]

N,N-Diisopropylethylamine (0.06 mL, 0.33 mmol) and magnesium chloride (12.0 mg, 0.13 mmol) were added to a mixture of Intermediate 4 (41.8 mg, 0.13 mmol) and Intermediate 51 (60.9 mg, 0.13 mmol) in tetrahydrofuran (1.5 mL) at RT. The mixture was heated to 55° C. After 5 h, the reaction mixture was allowed to cool to RT, diluted with ethyl acetate (20 mL) and the resulting mixture was washed with water (5×15 mL) and brine (20 mL). The organic layer was dried over anhydrous sodium sulfate and concentrated under reduced pressure. Concentrated aqueous hydrochloric acid solution (0.06 mL) was added dropwise to the crude residue in acetonitrile (1.5 mL) at 0° C. The mixture was warmed to RT. After 2 h, the reaction mixture was diluted with ethyl acetate (20 mL) and the resulting mixture was washed with saturated aqueous sodium carbonate solution (20 mL) and brine (20 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Methanol-d₄) δ 7.80 (d, J=3.7 Hz, 1H), 7.14 (dd, J=9.0, 1.4 Hz, 1H), 7.09-7.03 (m, 1H), 6.90-6.80 (m, 3H), 6.73 (dd, J=4.8, 1.0 Hz, 1H), 5.50 (dd, J=7.8, 5.0 Hz, 1H), 4.99-4.86 (m, 1H), 4.62 (q, J=5.1 Hz, 1H), 4.53-4.29 (m, 3H), 4.10-4.01 (m, 2H), 3.90-3.77 (m, 1H), 3.77-3.68 (m, 2H), 3.41 (d, J=2.1 Hz, 3H), 1.26 (ddd, J=7.1, 3.7, 1.1 Hz, 3H), 1.23-1.13 (m, 6H). ³¹P NMR (162 MHz, Methanol-d₄) δ 3.71. LCMS: MS m/z=635.19 [M+1], t_{R}=0.95 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.68 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

### Example 54. butyl ((S)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

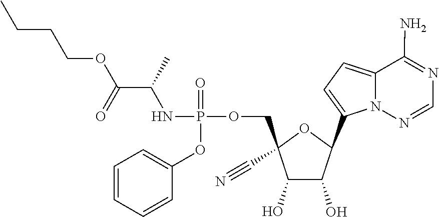 [SMILES: CCCCOC(=O)[C@H](C)NP(=O)(OC[C@@]1(C#N)O[C@@H](c2ccc3c(N)ncnn23)[C@H](O)[C@@H]1O)Oc1ccccc1]

N,N-diisopropylethylamine (0.13 mL, 0.76 mmol) and magnesium chloride (43 mg, 0.45 mmol) were added to a mixture of Intermediate 4 (100.0 mg, 0.30 mmol) and Intermediate 52 (191 mg, 0.45 mmol) in tetrahydrofuran (7.5 mL) at RT. The mixture was heated to 55° C. After 2 h, the reaction mixture was allowed to cool to RT, diluted with ethyl acetate (30 mL) and the resulting mixture was washed with water (5×20 mL) and brine (20 mL). The organic layer was dried over anhydrous sodium sulfate and concentrated under reduced pressure. Concentrated aqueous hydrochloric acid solution (0.53 mL) was added dropwise to the crude residue in acetonitrile (7.5 mL) at 0° C. The mixture was warmed to RT. After 2 h, the reaction mixture was diluted with ethyl acetate (100 mL) and the resulting mixture was washed with saturated aqueous sodium carbonate solution (75 mL) and brine (75 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Methanol-d₄) δ 7.79 (d, J=7.2 Hz, 1H), 7.37-7.10 (m, 4H), 6.84 (dd, J=4.5, 2.8 Hz, 1H), 6.73 (dd, J=4.5, 2.0 Hz, 1H), 5.49 (t, J=5.2 Hz, 1H), 4.62 (q, J=5.3 Hz, 1H), 4.55-4.28 (m, 3H), 4.15-3.80 (m, 3H), 1.68-1.46 (m, 2H), 1.46-1.22 (m, 5H), 0.99-0.83 (m, 3H). ³¹P NMR (162 MHz, Methanol-d₄) δ 3.25. LCMS: MS m/z=575.14 [M+1], t_{R}=0.83 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=6.50 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-19.0 min 2-95% ACN, 19.0 min-20.0 min 95% ACN at 2 mL/min.

### Example 55. tetrahydro-2H-pyran-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

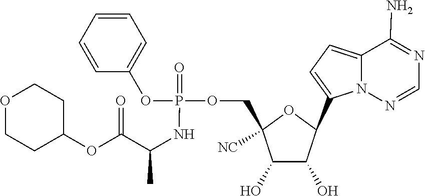 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCOCC1]

To a mixture of Intermediate 22 (1.7 g, 3.77 mmol), Intermediate 4 (1 g, 3 mmol), and MgCl₂ (359 mg, 3.77 mmol) in acetonitrile (40 mL) was added N,N-diisopropylethylamine (0.98 g, 8 mmol) dropwise. The resulting mixture was stirred at 50° C. for 2 h, reaction mixture was cooled, diluted with EtOAc, washed with water and brine, the organic solvent was evaporated under vacuum, the residue was then dissolved in acetonitrile, cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was purified by silica gel column chromatography eluting with 0-100% MeOH in DCM to afford the product. ¹H NMR (400 MHz, Methanol-d4) δ 7.80 (s, 1H), 7.78 (s, 1H), 7.33-7.24 (m, 2H), 7.24-7.10 (m, 3H), 6.84 (d, J=4.5 Hz, 1H), 6.73 (d, J=4.5 Hz, 1H), 5.50 (d, J=5.0 Hz, 1H), 4.62 (t, J=5.3 Hz, 1H), 4.54-4.42 (m, 2H), 4.35 (dd, J=10.9, 5.2 Hz, 1H), 3.97-3.80 (m, 3H), 3.56-3.44 (m, 2H), 1.89-1.81 (m, 2H), 1.60 (dtd, J=12.9, 8.6, 3.9 Hz, 2H), 1.27 (dd, J=7.2, 1.3 Hz, 4H), 1.14 (d, J=6.1 Hz, 5H). ³¹P NMR (162 MHz, Methanol-d4) δ 3.23. LCMS: MS m/z=603.14 [M+1], t_{R}=1.20 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.87 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 56. Single Diastereomer of tetrahydro-2H-pyran-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

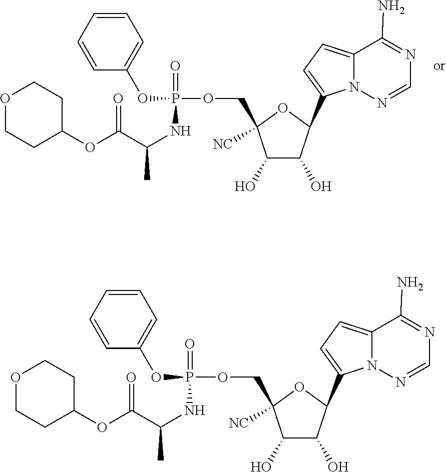 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCOCC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCOCC1]

First Eluting Diastereomer of Example 55: ¹H NMR (400 MHz, Methanol-d4) δ 7.78 (s, 1H), 7.29 (dd, J=8.7, 7.0 Hz, 2H), 7.16 (ddd, J=7.1, 2.1, 1.1 Hz, 3H), 6.85 (d, J=4.5 Hz, 1H), 6.73 (d, J=4.5 Hz, 1H), 5.50 (d, J=5.0 Hz, 1H), 4.88 (m, 1H), 4.63 (t, J=5.3 Hz, 1H), 4.55-4.44 (m, 2H), 4.36 (dd, J=10.9, 5.2 Hz, 1H), 3.86 (m, 3H), 3.50 (dtd, J=11.3, 5.4, 2.7 Hz, 2H), 1.94-1.76 (m, 2H), 1.60 (dtd, J=12.9, 8.4, 3.9 Hz, 2H), 1.27 (dd, J=7.1, 1.3 Hz, 3H). ³¹P NMR (162 MHz, Methanol-d4) δ 3.23. MS m/z=603 (M+H)⁺.

### Example 57. Single Diastereomer of tetrahydro-2H-pyran-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

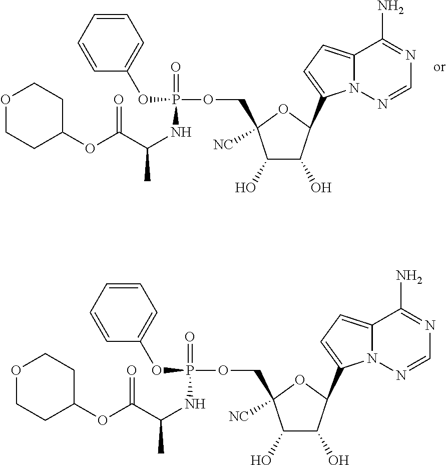 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCOCC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCOCC1]

Second eluting diastereomer of Example 55: ¹H NMR (400 MHz, Methanol-d4) δ 7.80 (s, 1H), 7.33 (dd, J=8.6, 7.2 Hz, 2H), 7.27-7.11 (m, 3H), 6.84 (d, J=4.5 Hz, 1H), 6.74 (d, J=4.5 Hz, 1H), 5.49 (d, J=5.0 Hz, 1H), 4.80 (m, 1H), 4.61 (t, J=5.3 Hz, 1H), 4.50-4.38 (m, 2H), 4.35 (dd, J=10.9, 5.5 Hz, 1H), 3.90 (dq, J=9.9, 7.1 Hz, 1H), 3.85-3.75 (m, 2H), 3.46 (dddd, J=11.8, 8.9, 6.0, 3.2 Hz, 2H), 1.81 (tdd, J=9.6, 4.6, 2.5 Hz, 2H), 1.57 (dtd, J=12.7, 8.4, 3.9 Hz, 2H), 1.27 (dd, J=7.1, 1.1 Hz, 3H). ³¹P NMR (162 MHz, Methanol-d4) δ 3.23. MS m/z=603 (M+H)⁺.

### Example 58. 3-Methoxypropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

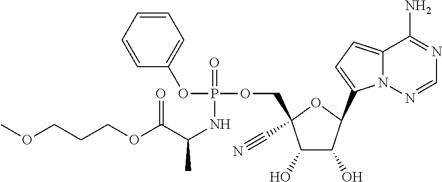 [SMILES: COCCCOC(=O)[C@H](C)NP(=O)(OC[C@@]1(C#N)O[C@@H](c2ccc3c(N)ncnn23)[C@H](O)[C@@H]1O)Oc1ccccc1]

To a mixture of Intermediate 4 (127 mg, 0.38 mmol), Intermediate 53 (252 mg, 0.58 mmol), and MgCl₂ (55 mg, 0.58 mmol) in THF (5 mL) was added N,N-diisopropylethylamine (0.17 mL, 0.97 mmol) dropwise at room temperature. The resulting mixture was stirred at 50° C. for 2 h and purified by preparative HPLC (Phenominex Gemini-NX 10 u C18 110 Å 250×30 mm column, ACN 10 to 100% in water). The obtained residue was dissolved in ACN (8 mL) and c-HCl (0.2 mL) added. The resulting mixture was stirred at room temperature for 1 h, cooled under ice bath, and aq.NaHCO₃ (4 mL) added slowly. The mixture was concentrated to half volume and purified by preparative HPLC (Phenominex Gemini-NX 10 u C18 110 Å 250×30 mm column, ACN 10 to 100% in water) to afford the product. ¹H NMR (400 MHz, Methanol-d4) δ 7.80 (s, 0.64H), 7.78 (s, 0.36H), 7.31 (m, 2H), 7.25-7.12 (m, 3H), 6.84 (m, 1H), 6.73 (m, 1H), 5.50 (m, 1H), 4.62 (m, 1H), 4.53-4.38 (m, 2H), 4.34 (m, 1H), 4.17-4.00 (m, 2H), 3.93-3.83 (m, 1H), 3.39 (m, 2H), 3.27 (m, 3H), 1.81 (m, 2H), 1.26 (m, 3H). ³¹P NMR (162 MHz, Methanol-d4) δ 3.24. LCMS: m/z=591.18 (M+H), t_{R}=0.96 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.96 min (35%) and 4.02 min (64%); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

Resolution of the Sp and Rp diastereomers. The product was separated by IA SFC 5 um, 21×250 mm (30% 2-propanol) to afford the diastereomers:

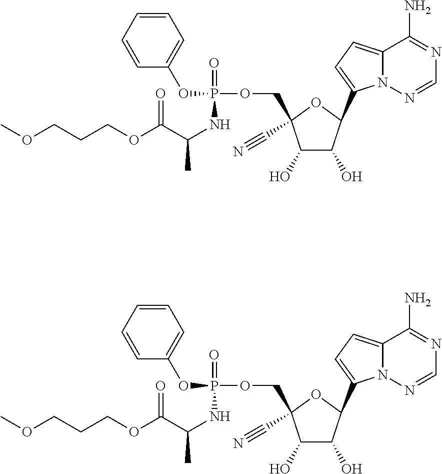 [SMILES: COCCCOC(=O)[C@H](C)N[P@@](=O)(OC[C@@]1(C#N)O[C@@H](c2ccc3c(N)ncnn23)[C@H](O)[C@@H]1O)Oc1ccccc1 ; COCCCOC(=O)[C@H](C)N[P@](=O)(OC[C@@]1(C#N)O[C@@H](c2ccc3c(N)ncnn23)[C@H](O)[C@@H]1O)Oc1ccccc1]

### Example 59. First Eluting Diastereomer of Example 58

¹H NMR (400 MHz, Methanol-d4) δ 7.78 (s, 1H), 7.33-7.25 (m, 2H), 7.19-7.12 (m, 3H), 6.84 (d, J=4.5 Hz, 1H), 6.72 (d, J=4.5 Hz, 1H), 5.51 (d, J=5.0 Hz, 1H), 4.63 (t, J=5.3 Hz, 1H), 4.52 (d, J=5.6 Hz, 1H), 4.47 (dd, J=10.9, 6.0 Hz, 1H), 4.35 (dd, J=10.9, 5.1 Hz, 1H), 4.12 (td, J=6.5, 2.1 Hz, 2H), 3.89 (ddd, J=14.4, 10.8, 6.6 Hz, 1H), 3.39 (t, J=6.2 Hz, 2H), 3.26 (s, 3H), 1.83 (m, 2H), 1.25 (dd, J=7.1, 1.2 Hz, 3H). ³¹P NMR (162 MHz, Methanol-d4) δ 3.24. HPLC: t_{R}=3.96 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 60. Second Eluting Diastereomer of Example 58

¹H NMR (400 MHz, Methanol-d4) δ 7.79 (s, 1H), 7.32 (dd, J=8.6, 7.2 Hz, 2H), 7.22 (dt, J=8.6, 1.3 Hz, 2H), 7.20-7.13 (m, 1H), 6.83 (d, J=4.5 Hz, 1H), 6.73 (d, J=4.5 Hz, 1H), 5.50 (d, J=5.0 Hz, 1H), 4.62 (t, J=5.3 Hz, 1H), 4.47 (d, J=5.6 Hz, 1H), 4.41 (dd, J=10.9, 6.3 Hz, 1H), 4.34 (dd, J=10.9, 5.5 Hz, 1H), 4.07 (qt, J=10.9, 6.4 Hz, 2H), 3.90 (dq, J=10.0, 7.1 Hz, 1H), 3.37 (t, J=6.2 Hz, 2H), 3.25 (s, 3H), 1.79 (m, 2H), 1.26 (dd, J=7.2, 1.0 Hz, 3H). ³¹P NMR (162 MHz, Methanol-d4) δ 3.24. HPLC: t_{R}=4.02 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 61. (R)-1-methylpyrrolidin-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

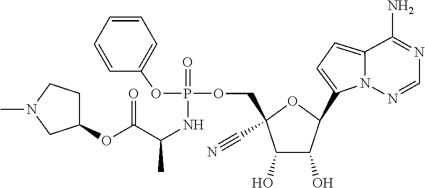 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCN(C)C1]

Intermediate 4 (50 mg, 0.15 mmol) and Intermediate 24 (81 mg, 0.18 mmol) were mixed and dissolved in 1.5 mL of anhydrous THF. Magnesium chloride (43 mg, 0.45 mmol) was added in one portion. DIPEA (65 uL, 0.375 mmol) was added, and the reaction was stirred at 50° C. for 16 hrs.

Reaction was diluted with EtOAc (15 mL) and washed with water (6×10 mL) and then with brine (5 mL). Organic extract was dried over anhydrous sodium sulfate and concentrated under reduced pressure. The crude residue was purified via SiO₂ column chromatography (4 g SiO₂ Combiflash HP Gold Column, 0-5-10-20% methanol/DCM). Fractions having the desired product were combined and concentrated under reduced pressure. Residue was dissolved in MeCN (5 mL) and stirred in an ice bath. Concentrate aqueous hydrochloric acid (300 uL) was added dropwise. Reaction was stirred in an ice bath for 2 hrs. Reaction was diluted with EtOAc (20 mL) and added saturated aqueous sodium bicarbonate solution (30 mL). Mixture was stirred for 10 mins. Organic extract was collected and aqueous portion was extracted with EtOAc (2×10 mL). Organic extracts were combined, dried over anhydrous sodium sulfate and concentrated under reduced pressure. Residue was dissolved in MeCN and water and freeze-dried to give the product. ¹H NMR (400 MHz, methanol-d₄) δ 7.80 (m, 1H), 7.41-7.09 (m, 5H), 6.85 (m, 1H), 6.74 (m, 1H), 5.49 (m, 1H), 5.33-5.15 (m, 1H), 4.70-4.58 (m, 1H), 4.56-4.28 (m, 3H), 4.00-3.86 (m, 1H), 3.28-3.07 (m, 3H), 3.03-2.83 (m, 1H), 2.69 (m, 3H), 2.35 (m, 1H), 2.00 (m, 1H), 1.28 (m, 3H). ³¹P NMR (162 MHz, methanol-d₄) δ 3.39, 3.05. LCMS: MS m/z=602.2 [M+1], 599.9 [M−1], t_{R}=1.00 min; LC system: Thermo Dionex Ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=1.85 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.142, 3.190 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 62. methyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)oxy)phenyl)-2-(((benzyloxy)carbonyl)amino)propanoate

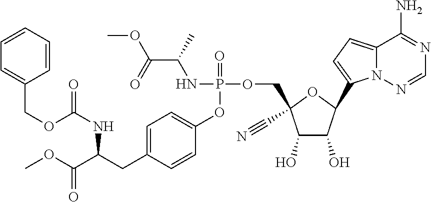 [SMILES: COC(=O)[C@H](C)NP(=O)(OC[C@@]1(C#N)O[C@@H](c2ccc3c(N)ncnn23)[C@H](O)[C@@H]1O)Oc1ccc(C[C@H](NC(=O)OCc2ccccc2)C(=O)OC)cc1]

methyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)oxy)phenyl)-2-(((benzyloxy)carbonyl)amino)propanoate. N,N-Diisopropylethylamine (0.11 mL, 0.604 mmol) and magnesium chloride (23 mg, 0.24 mmol) were added to a mixture of Intermediate 4 (80 mg, 0.24 mmol) and Intermediate 54 (178 mg, 0.29 mmol) in tetrahydrofuran (3.8 mL) at RT. The mixture was heated to 55° C. After 2 h, the reaction mixture was allowed to cool to RT, diluted with ethyl acetate (50 mL) and the resulting mixture was washed with water (5×50 mL) and brine (50 mL). The organic layer was dried over anhydrous sodium sulfate and concentrated under reduced pressure. Concentrated aqueous hydrochloric acid solution (0.11 mL) was added dropwise to the crude residue in acetonitrile (3.8 mL) at 0° C. The mixture was warmed to RT. After 3.5 h, the reaction mixture was diluted with ethyl acetate (50 mL) and the resulting mixture was washed with saturated aqueous sodium carbonate solution (2×50 mL) and brine (50 mL). The organic layer was dried over anhydrous sodium sulfate and concentrated under reduced pressure. The crude residue was subjected to silica gel chromatography eluting with 0-25% methanol in dichloromethane to afford the product. ¹H NMR (400 MHz, Methanol-d₄) δ 7.78 (d, J=9.1 Hz, 1H), 7.38-7.25 (m, 5H), 7.19-7.09 (m, 3H), 7.06 (dd, J=8.7, 1.2 Hz, 1H), 6.84 (dd, J=4.5, 1.3 Hz, 1H), 6.72 (dd, J=7.2, 4.5 Hz, 1H), 5.56-5.46 (m, 1H), 5.03 (d, J=2.9 Hz, 2H), 4.63 (td, J=5.3, 4.4 Hz, 1H), 4.54-4.29 (m, 4H), 3.87 (ddq, J=16.7, 9.4, 7.1 Hz, 1H), 3.69 (d, J=3.0 Hz, 3H), 3.61 (d, J=15.9 Hz, 4H), 3.20-3.06 (m, 1H), 2.91 (dt, J=14.0, 8.4 Hz, 1H), 1.24 (td, J=7.1, 1.1 Hz, 3H). ³¹P NMR (162 MHz, Methanol-d₄) δ 3.27 (d, J=2.1 Hz). LCMS: MS m/z=768.49 [M+1], t_{R}=1.12 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.95 min, 4.02 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

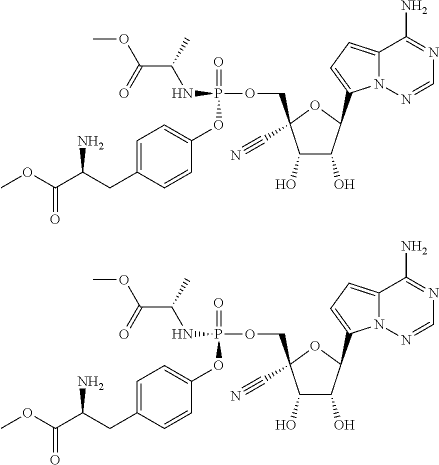 [SMILES: COC(=O)[C@H](C)N[P@@](=O)(OC[C@@]1(C#N)O[C@@H](c2ccc3c(N)ncnn23)[C@H](O)[C@@H]1O)Oc1ccc(C[C@H](N)C(=O)OC)cc1 ; COC(=O)[C@H](C)N[P@](=O)(OC[C@@]1(C#N)O[C@@H](c2ccc3c(N)ncnn23)[C@H](O)[C@@H]1O)Oc1ccc(C[C@H](N)C(=O)OC)cc1]

methyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)oxy)phenyl)-2-(((benzyloxy)carbonyl)amino)propanoate. Palladium on carbon (10.3 mg, 10 wt %) was added to a solution of methyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)oxy)phenyl)-2-(((benzyloxy)carbonyl)amino)propanoate (30.6 mg, 0.04 mmol) in ethanol (5 mL) that was purged with argon. The mixture was then purged with hydrogen and stirred at RT. After 18 hr, the mixture was filtered through celite, the filter was rinsed with ethanol, and the volatiles were removed under reduce pressure. The crude residue was subjected to preparatory HPLC (Phenomenex Synergi 4 um Polar-RP 80 Å 150×21.2 mm column, 10-60% acetonitrile/water gradient with 0.1% TFA) to afford the product as TFA salts.

### Example 62. First Eluting Diastereomer

¹H NMR (400 MHz, Methanol-d₄) δ 7.95 (s, 1H), 7.29-7.16 (m, 5H), 6.93 (s, 1H), 5.53 (d, J=5.3 Hz, 1H), 4.59 (t, J=5.4 Hz, 1H), 4.52-4.43 (m, 2H), 4.37 (dd, J=10.9, 5.2 Hz, 1H), 4.30 (dd, J=7.6, 6.1 Hz, 1H), 4.03-3.87 (m, 1H), 3.81 (s, 3H), 3.69 (s, 3H), 3.25 (dd, J=14.5, 6.1 Hz, 1H), 3.13 (dd, J=14.6, 7.4 Hz, 1H), 1.34 (dd, J=7.4, 1.2 Hz, 3H). ¹⁹F NMR (376 MHz, Methanol-d₄) δ −77.68. ³¹P NMR (162 MHz, Methanol-d₄) δ 3.54. LCMS: MS m/z=634.18 [M+1], t_{R}=0.77 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=2.30 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Example 63. Second Eluting Diastereomer

¹H NMR (400 MHz, Methanol-d₄) δ 7.94 (s, 1H), 7.32-7.22 (m, 4H), 7.14 (s, 1H), 6.89 (s, 1H), 5.52 (d, J=4.9 Hz, 1H), 4.58 (t, J=5.3 Hz, 1H), 4.40 (dd, J=12.5, 5.8 Hz, 2H), 4.37-4.28 (m, 2H), 3.92 (dd, J=10.0, 7.3 Hz, 1H), 3.83 (s, 3H), 3.61 (s, 3H), 3.27-3.08 (m, 2H), 1.31 (d, J=7.1 Hz, 3H). ¹⁹F NMR (376 MHz, Methanol-d₄) δ −77.65. ³¹P NMR (162 MHz, Methanol-d₄) δ 3.48. LCMS: MS m/z=634.24 [M+1], t_{R}=0.80 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=2.43 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Example 64. (S)-Tetrahydrofuran-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

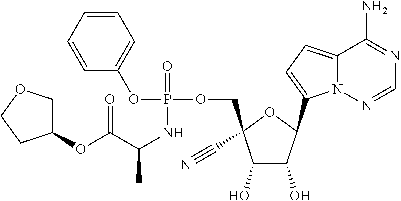 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCOC1]

To a mixture of Intermediate 4 (132 mg, 0.40 mmol), Intermediate 55 (234 mg, 0.54 mmol), and MgCl₂ (46 mg, 0.48 mmol) in THF (5 mL) was added N,N-diisopropylethylamine (0.10 mL, 0.60 mmol) dropwise at room temperature. The resulting mixture was stirred at 50° C. for 2 h and purified by preparative HPLC (Phenominex Gemini-NX 10 u C18 110 Å 250×30 mm column, ACN 10 to 100% in water). The obtained residue was dissolved in ACN (4 mL) and c-HCl (0.2 mL) added. The resulting mixture was stirred at room temperature for 1 h, cooled under ice bath, neutralized with 5 N NaOH, and purified by preparative HPLC (Phenominex Gemini-NX 10 u C18 110 Å 250×30 mm column, ACN 10 to 100% in water) to afford the product. ¹H NMR (400 MHz, Methanol-d₄) δ 7.80 (s, 0.67H), 7.78 (s, 0.33H), 7.37-7.13 (m, 5H), 6.84 (m, 1H), 6.73 (m, 1H), 5.49 (m, 1H), 5.25-5.20 (m, 0.33H), 5.18-5.10 (m, 0.67H), 4.62 (m, 1H), 4.53-4.30 (m, 3H), 3.93-3.63 (m, 5H), 2.20-1.99 (m, 1H), 1.98-1.87 (m, 1H), 1.25 (m, 3H). LCMS: m/z=589.02 (M+H), t_{R}=1.06 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.75 min (29%), 3.81 min (68%); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

Resolution of the Sp and Rp diastereomers. The mixture was separated by Chiralpak AD-H, 150×4.6 mm, 5 um (100% EtOH).

### Example 65. Single Diastereomer of (S)-Tetrahydrofuran-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

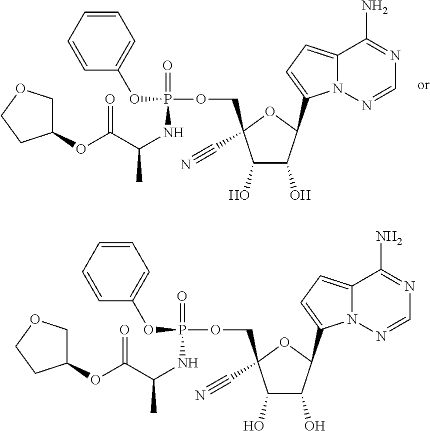 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CCOC1 ; C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CCOC1]

First eluting diastereomer of Example 64: ¹H NMR (400 MHz, Methanol-d4) δ 7.78 (s, 1H), 7.28 (m, 2H), 7.16 (dt, J=8.1, 1.3 Hz, 3H), 6.84 (d, J=4.5 Hz, 1H), 6.73 (d, J=4.6 Hz, 1H), 5.50 (d, J=5.0 Hz, 1H), 5.23 (t, J=5.5 Hz, 1H), 4.63 (t, J=5.3 Hz, 1H), 4.51 (d, J=5.5 Hz, 1H), 4.47 (dd, J=10.9, 5.9 Hz, 1H), 4.35 (dd, J=10.9, 5.2 Hz, 1H), 3.92-3.68 (m, 5H), 2.23-2.06 (m, 1H), 2.01-1.91 (m, 1H), 1.25 (dd, J=7.1, 1.3 Hz, 3H). ³¹P NMR (162 MHz, Methanol-d4) δ 3.22. LCMS: m/z=589.02 (M+H), t_{R}=1.06 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.75 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 66. Single Diastereomer of (S)-Tetrahydrofuran-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

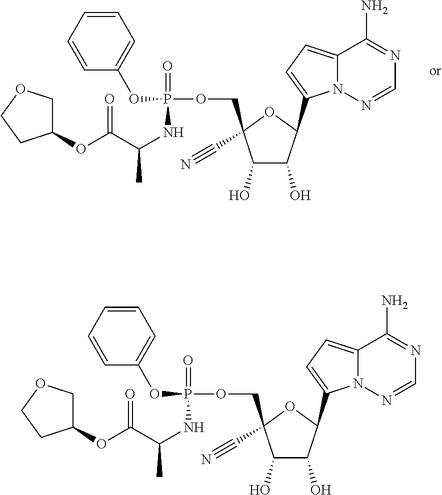 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CCOC1 ; C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CCOC1]

Second eluting diastereomer of Example 64: ¹H NMR (400 MHz, Methanol-d4) δ 7.80 (s, 1H), 7.33 (dd, J=8.6, 7.2 Hz, 2H), 7.25-7.21 (m, 2H), 7.20-7.15 (m, 1H), 6.84 (d, J=4.5 Hz, 1H), 6.74 (d, J=4.5 Hz, 1H), 5.48 (d, J=5.0 Hz, 1H), 5.14 (dd, J=6.0, 4.1 Hz, 1H), 4.62 (t, J=5.3 Hz, 1H), 4.46 (d, J=5.7 Hz, 1H), 4.41 (dd, J=10.9, 6.4 Hz, 1H), 4.33 (dd, J=10.9, 5.4 Hz, 1H), 3.95-3.65 (m, 5H), 2.11-1.98 (m, 1H), 1.96-1.82 (m, 1H), 1.25 (dd, J=7.1, 1.1 Hz, 3H). ³¹P NMR (162 MHz, Methanol-d4) δ 3.19. LCMS: m/z=589.02 (M+H), t_{R}=1.07 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.82 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 67. 3-morpholinopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

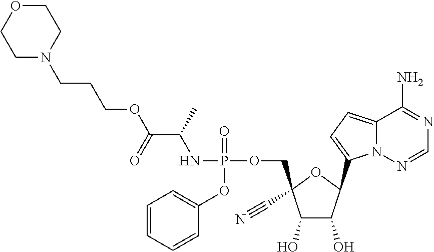 [SMILES: C[C@H](NP(=O)(OC[C@@]1(C#N)O[C@@H](c2ccc3c(N)ncnn23)[C@H](O)[C@@H]1O)Oc1ccccc1)C(=O)OCCCN1CCOCC1]

N,N-Diisopropylethylamine (0.11 mL, 0.62 mmol) and magnesium chloride (23.8 mg, 0.25 mmol) were added to a mixture of Intermediate 4 (82.7 mg, 0.25 mmol) and Intermediate 56 (133 mg, 0.27 mmol) in tetrahydrofuran (2.5 mL) at RT. The mixture was heated to 55° C. After 4.5 h, the reaction mixture was allowed to cool to RT, diluted with ethyl acetate (25 mL) and the resulting mixture was washed with water (2×15 mL) and brine (20 mL). The organic layer was dried over anhydrous sodium sulfate and concentrated under reduced pressure. Concentrated aqueous hydrochloric acid solution (0.12 mL) was added dropwise to the crude residue in acetonitrile (5 mL). After 4.5 h, the reaction mixture was concentrated under reduced pressure. The resulting crude residue was subjected preparatory HPLC (Phenomenex Gemini 10 u C18 110 Å AXIA 250×21.2 mm column, 30-70% acetonitrile/water gradient with 0.1% TFA) followed by silica gel chromatography eluting with 0-25% methanol in dichloromethane to afford the product. ¹H NMR (400 MHz, Acetonitrile-d₃) δ 7.94 (s, 1H), 7.43-7.19 (m, 6H), 5.51 (d, J=4.4 Hz, 1H), 4.55-4.43 (m, 3H), 4.43-4.33 (m, 2H), 4.09 (dt, J=9.4, 4.9 Hz, 2H), 3.95 (d, J=13.1 Hz, 3H), 3.77 (m, 2H), 3.38 (s, 2H), 3.13 (q, J=8.7, 7.9 Hz, 2H), 2.08-1.99 (m, 2H), 1.30 (t, J=8.6 Hz, 3H). ³¹P NMR (162 MHz, Methanol-d₄) δ 2.70, 2.40. LCMS: MS m/z=646.35 [M+1], t_{R}=1.05 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.23 min; HPLC system: Agilent 1100 series; Column: Kinetx 2.6 u 100 A C18, 100 mm×4.6 mm; Solvents: Acetonitrile with 0.1% TFA, Water with 0.1% TFA; Gradient: 0 min-8.5 min 2-98% ACN, 8.5 min-10.0 min 98% ACN at 1.5 mL/min.

### Example 68. (R)-Tetrahydrofuran-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

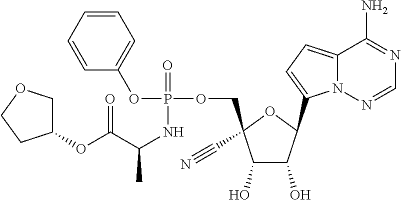 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCOC1]

To a mixture of Intermediate 4 (130 mg, 0.40 mmol), Intermediate 57 (256 mg, 0.59 mmol), and MgCl₂ (46 mg, 0.48 mmol) in THF (5 mL) was added N,N-diisopropylethylamine (0.10 mL, 0.60 mmol) dropwise at room temperature. The resulting mixture was stirred at 50° C. for 2 h and purified by preparative HPLC (Phenominex Gemini-NX 10 u C18 110 Å 250×30 mm column, ACN 10 to 100% in water). The obtained residue was dissolved in ACN (4 mL) and c-HCl (0.2 mL) added. The resulting mixture was stirred at room temperature for 1 h, cooled under ice bath, neutralized with 5 N NaOH, and purified by preparative HPLC (Phenominex Gemini-NX 10 u C18 110 Å 250×30 mm column, ACN 10 to 100% in water) to afford the product. ¹H NMR (400 MHz, Methanol-d4) δ 7.80 (s, 0.71H), 7.78 (s, 0.29H), 7.31 (m, 2H), 7.25-7.13 (m, 3H), 6.84 (m, 1H), 6.73 (m, 1H), 5.49 (m, 1H), 5.23 (s, 0.29H), 5.20-5.14 (m, 0.71H), 4.66-4.59 (m, 1H), 4.53-4.30 (m, 3H), 3.95-3.69 (m, 5H), 2.22-2.05 (m, 1H), 1.99-1.85 (m, 1H), 1.25 (m, 3H). ³¹P NMR (162 MHz, Methanol-d4) δ 3.22, 3.17. LCMS: m/z=589.03 (M+H), t_{R}=1.07 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.77 min (25%), 3.82 min (75%); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min

Resolution of the Sp and Rp diastereomers. The mixture was separated by Chiralpak IA (150×4.6 mm, 5 micron. 100% EtOH) to afford the diastereomers:

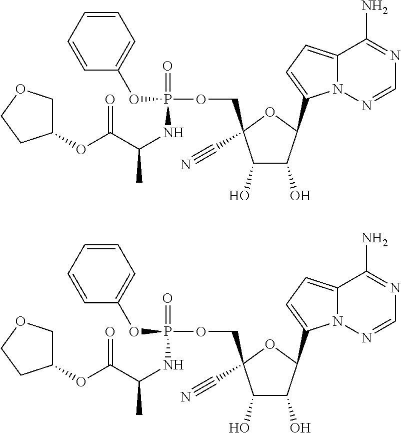 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@@H]1CCOC1 ; C[C@H](N[P@](=O)(OC[C@@]1(C#N)O[C@@H](c2ccc3c(N)ncnn23)[C@H](O)[C@@H]1O)Oc1ccccc1)C(=O)O[C@@H]1CCOC1]

### Example 69. First Eluting Diastereomer of Example 68

¹H NMR (400 MHz, Methanol-d4) δ 7.78 (s, 1H), 7.32-7.26 (m, 2H), 7.19-7.13 (m, 3H), 6.84 (d, J=4.5 Hz, 1H), 6.73 (d, J=4.6 Hz, 1H), 5.50 (d, J=5.0 Hz, 1H), 5.26-5.20 (m, 1H), 4.62 (t, J=5.3 Hz, 1H), 4.50 (d, J=5.6 Hz, 1H), 4.47 (dd, J=11.0, 6.0 Hz, 1H), 4.35 (dd, J=10.9, 5.2 Hz, 1H), 3.94-3.69 (m, 5H), 2.15 (td, J=14.5, 8.3 Hz, 1H), 1.99-1.86 (m, 1H), 1.25 (dd, J=7.2, 1.2 Hz, 3H). ³¹P NMR (162 MHz, Methanol-d4) δ 3.22. LCMS: m/z=589.09 (M+H), t_{R}=0.95 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.76 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min

### Example 70. Second Eluting Diastereomer of Example 68

¹H NMR (400 MHz, Methanol-d4) δ 7.80 (s, 1H), 7.40-7.25 (m, 3H), 7.28-7.12 (m, 2H), 6.84 (d, J=4.5 Hz, 1H), 6.74 (d, J=4.5 Hz, 1H), 5.49 (d, J=5.1 Hz, 1H), 5.17 (td, J=4.1, 2.1 Hz, 1H), 4.62 (t, J=5.3 Hz, 1H), 4.46 (d, J=5.6 Hz, 1H), 4.41 (dd, J=10.9, 6.4 Hz, 1H), 4.34 (dd, J=10.9, 5.4 Hz, 1H), 3.98-3.68 (m, 5H), 2.18-2.03 (m, 1H), 1.96-1.83 (m, 1H), 1.25 (dd, J=7.2, 1.1 Hz, 3H). ³¹P NMR (162 MHz, Methanol-d4) δ 3.17. LCMS: m/z=589.10 (M+H), t_{R}=0.96 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.81 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 71. methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphorothioyl)-L-alaninate

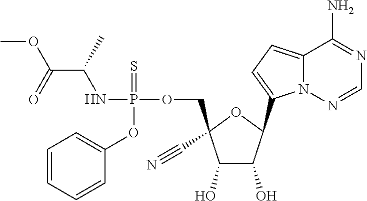

Triethylamine (170 μl, 1.2 mmol) was added to a solution of Intermediate 4 (0.40 g, 1.2 mmol) and Intermediate 58 (0.35 g, 1.2 mmol) in acetonitrile (6 mL) at RT. The reaction mixture was warmed to 65° C. After 3 h, the reaction mixture was allowed to cool to RT and concentrated aqueous hydrochloric acid solution (300 μL) was added. After 1 h, saturated aqueous sodium bicarbonate solution (5 mL) was slowly added and the resulting mixture was extracted with dichloromethane (3×5 mL). The combined organic extracts were dried over anhydrous sodium sulfate and were concentrated under reduced pressure. The crude residue was subjected to silica gel chromatography eluting with 0-100% ethyl acetate in hexanes to afford the product. ¹H NMR (400 MHz, methanol-d₄) δ 7.95 (br s, 1H), 7.40-7.02 (m, 5H), 6.90-6.72 (m, 2H), 5.52-5.45 (m, 1H), 4.58-4.49 (m, 1H), 4.43-4.30 (m, 2H), 3.90-3.77 (m, 2H), 3.71-3.54 (m, 3H), 1.38-1.29 (m, 3H). LCMS: MS m/z=549.27 [M+1], t_{R}=1.23 min (minor isomer), 1.25 (major isomer); LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.21 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=5.124 min (minor isomer), 5.221 min (major isomer); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 72. methyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-isopropoxy-1-oxopropan-2-yl)amino)phosphoryl)oxy)phenyl)-2-(((benzyloxy)carbonyl)amino)propanoate

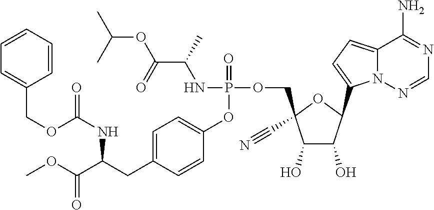 [SMILES: COC(=O)[C@H](Cc1ccc(OP(=O)(N[C@@H](C)C(=O)OC(C)C)OC[C@@]2(C#N)O[C@@H](c3ccc4c(N)ncnn34)[C@H](O)[C@@H]2O)cc1)NC(=O)OCc1ccccc1]

N,N-Diisopropylethylamine (0.06 mL, 0.33 mmol) and magnesium chloride (12.3 mg, 0.13 mmol) were added to a mixture of Intermediate 4 (42.7 mg, 0.13 mmol) and Intermediate 66 (82.9 mg, 0.13 mmol) in tetrahydrofuran (1.5 mL) at RT. The mixture was heated to 55° C. After 4 h, the reaction mixture was allowed to cool to RT, diluted with ethyl acetate (20 mL) and the resulting mixture was washed with water (5×15 mL) and brine (20 mL). The organic layer was dried over anhydrous sodium sulfate and concentrated under reduced pressure. Concentrated aqueous hydrochloric acid solution (0.06 mL) was added dropwise to the crude residue in acetonitrile (1.5 mL) at 0° C. The mixture was warmed to RT. After 2 h, the reaction mixture was diluted with ethyl acetate (20 mL) and the resulting mixture was washed with saturated aqueous sodium carbonate solution (20 mL) and brine (20 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Methanol-d₄) δ 7.79 (d, J=8.0 Hz, 1H), 7.31 (ddd, J=11.2, 6.2, 3.3 Hz, 5H), 7.20-7.10 (m, 3H), 7.06 (d, J=8.3 Hz, 1H), 6.87-6.80 (m, 1H), 6.72 (dd, J=5.5, 4.7 Hz, 1H), 5.56-5.44 (m, 1H), 5.03 (d, J=3.6 Hz, 2H), 4.91 (ddd, J=24.9, 12.6, 6.3 Hz, 1H), 4.62 (t, J=5.4 Hz, 1H), 4.48 (dd, J=11.8, 5.5 Hz, 1H), 4.45-4.28 (m, 3H), 3.90-3.77 (m, 1H), 3.69 (d, J=3.5 Hz, 3H), 3.16-3.05 (m, 1H), 2.91 (dt, J=14.0, 8.8 Hz, 1H), 1.25 (dt, J=7.2, 1.4 Hz, 3H), 1.20 (d, J=6.3 Hz, 3H), 1.16 (t, J=6.3 Hz, 3H). ³¹P NMR (162 MHz, Methanol-d₄) δ 3.36, 3.33. LCMS: MS m/z=796.45 [M+1], t_{R}=1.17 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.44 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Example 73. isopropyl (2S)-3-(4-(((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)oxy)phenyl)-2-(((benzyloxy)carbonyl)amino)propanoate

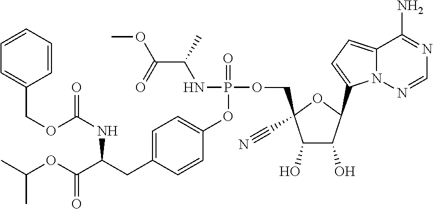 [SMILES: COC(=O)[C@H](C)NP(=O)(OC[C@@]1(C#N)O[C@@H](c2ccc3c(N)ncnn23)[C@H](O)[C@@H]1O)Oc1ccc(C[C@H](NC(=O)OCc2ccccc2)C(=O)OC(C)C)cc1]

N,N-Diisopropylethylamine (0.11 mL, 0.0.62 mmol) and magnesium chloride (23.8 mg, 0.25 mmol) were added to a mixture of Intermediate 4 (82.7 mg, 0.25 mmol) and Intermediate 69 (176.6 mg, 0.27 mmol) in tetrahydrofuran (2.5 mL) at RT. The mixture was heated to 55° C. After 4.5 h, the reaction mixture was allowed to cool to RT, diluted with ethyl acetate (25 mL) and the resulting mixture was washed with water (2×15 mL) and brine (20 mL). The organic layer was dried over anhydrous sodium sulfate and concentrated under reduced pressure. Concentrated aqueous hydrochloric acid solution (0.12 mL) was added dropwise to the crude residue in acetonitrile (5 mL). After 4.5 h, the reaction mixture was diluted with ethyl acetate (25 mL) and the resulting mixture was washed with saturated aqueous sodium carbonate solution (2×20 mL) and brine (20 mL). The organic layer was dried over anhydrous sodium sulfate and concentrated under reduced pressure. The crude residue was subjected to silica gel chromatography eluting with 0-25% methanol in dichloromethane to afford the product. ¹H NMR (400 MHz, Methanol-d₄) δ 7.79 (d, J=9.3 Hz, 1H), 7.36-7.21 (m, 5H), 7.20-7.10 (m, 3H), 7.09-7.03 (m, 1H), 6.84 (dd, J=4.5, 1.0 Hz, 1H), 6.73 (dd, J=7.4, 4.5 Hz, 1H), 5.51 (t, J=4.8 Hz, 1H), 5.04 (d, J=2.2 Hz, 2H), 5.01-4.89 (m, 1H), 4.66-4.60 (m, 1H), 4.55-4.28 (m, 4H), 3.87 (ddd, J=16.3, 9.6, 7.1 Hz, 1H), 3.61 (d, J=16.9 Hz, 3H), 3.09 (dt, J=14.2, 5.8 Hz, 1H), 2.91 (dt, J=15.3, 8.2 Hz, 1H), 1.24 (m, 6H), 1.16 (dd, J=6.3, 3.8 Hz, 3H). ³¹P NMR (162 MHz, Methanol-d₄) δ 3.27. LCMS: MS m/z=796.51 [M+1], t_{R}=1.25 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.331 min, 4.395 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

### Example 74. 2-(diisopropylamino)ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

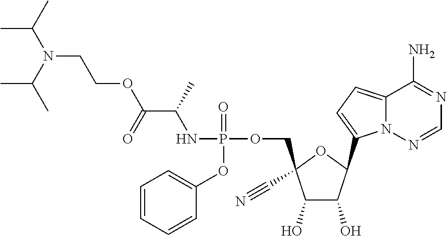 [SMILES: CC(C)N(CCOC(=O)[C@H](C)NP(=O)(OC[C@@]1(C#N)O[C@@H](c2ccc3c(N)ncnn23)[C@H](O)[C@@H]1O)Oc1ccccc1)C(C)C]

N,N-Diisopropylethylamine (0.20 mL, 1.17 mmol) and magnesium chloride (44.7 mg, 0.47 mmol) were added to a mixture of Intermediate 4 (155.6 mg, 0.47 mmol) and Intermediate 68 (231.8 mg, 0.47 mmol) in tetrahydrofuran (5.47 mL) at RT. The mixture was heated to 55° C. After 2 h, the reaction mixture was allowed to cool to RT, diluted with ethyl acetate (50 mL) and the resulting mixture was washed with water (2×50 mL) and brine (50 mL). The organic layer was dried over anhydrous sodium sulfate and concentrated under reduced pressure. Concentrated aqueous hydrochloric acid solution (0.40 mL) was added dropwise to the crude residue in acetonitrile (5 mL) at 0° C. The mixture was warmed to RT. After 20 h, the volatiles were removed under reduced pressure. The aqueous was lyophilized to afford the product that was used without further purification. LCMS: MS m/z=635.19 [M+1], t_{R}=0.95 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

### Example 75. 2-(2-ethoxyethoxy)ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate single isomer

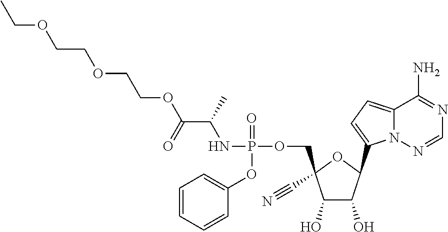 [SMILES: CCOCCOCCOC(=O)[C@H](C)NP(=O)(OC[C@@]1(C#N)O[C@@H](c2ccc3c(N)ncnn23)[C@H](O)[C@@H]1O)Oc1ccccc1]

Tetrahydrofuran (1.4 mL) was added to a mixture of Intermediate 4 (202 mg, 0.610 mmol), Intermediate 73 (418 mg, 0.793 mmol), and magnesium chloride (87 mg, 0.914 mmol) at room temperature. The mixture was heated to 40° C. for 10 min, and N,N-diisopropylethylamine (0.265 mL, 1.524 mmol) was added. After stirring for 2 hours at 40° C., the reaction mixture was allowed to cool to at room temperature, and was concentrated down under reduced pressure. The crude residue was dissolved in ethyl acetate (40 mL) and the resulting mixture was washed with water (30 mL) and brine (30 mL). The organic layer was separated, dried over anhydrous sodium sulfate and concentrated under reduced pressure. The crude residue was dissolved in acetonitrile (10 mL) and concentrated aqueous hydrochloric acid solution (0.508 mL) was added dropwise at 0° C. After 4 hours at 0° C., the reaction mixture was diluted with ethyl acetate (50 mL) and water (30 mL) at 0° C. and the resulting mixture was washed with saturated aqueous sodium bicarbonate solution (20 mL) and brine (20 mL). The organic layer was dried over anhydrous sodium sulfate and concentrated under reduced pressure. The crude residue was purified by preparatory HPLC (Phenomenex Gemini 5 μm C18 110 Å 100×30 mm column) using gradient from 10-100% acetonitrile in water to afford the product. ¹H NMR (400 MHz, DMSO-d₆) δ 7.86 (s, 1H), 7.77 (bs, 2H), 7.41-7.32 (m, 2H), 7.27-7.12 (m, 3H), 6.85 (d, J=4.4 Hz, 1H), 6.74 (d, J=4.5 Hz, 1H), 6.25-6.07 (m, 2H), 5.50 (d, J=5.9 Hz, 1H), 5.38 (d, J=6.2 Hz, 1H), 4.54-4.42 (m, 1H), 4.35-4.21 (m, 2H), 4.22-4.08 (m, 2H), 4.07-3.95 (m, 1H), 3.92-3.77 (m, 1H), 3.55-3.49 (m, 2H), 3.49-3.44 (m, 2H), 3.43-3.36 (m, 4H), 1.20 (d, J=7.1 Hz, 3H), 1.07 (t, J=7.0 Hz, 3H). ³¹P NMR (162 MHz, DMSO-d₆) δ 3.24. LCMS: MS m/z=635.07 [M+1], t_{R}=1.17 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-0.2 min 2% acetonitrile, 0.2 min-1.5 min 2-100% acetonitrile, 1.5 min-2.2 min 100% acetonitrile, 2.2 min-2.4 min 100%-2% acetonitrile, 2.4 min-2.5 min 2% acetonitrile at 2 μL/min. HPLC: t_{R}=2.45 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 m/min. HPLC: t_{R}=4.09 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 76. methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(benzyloxy)phosphoryl)-L-alaninate

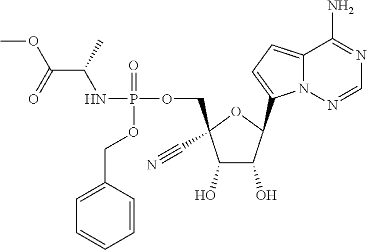 [SMILES: COC(=O)[C@H](C)NP(=O)(OCc1ccccc1)OC[C@@]1(C#N)O[C@@H](c2ccc3c(N)ncnn23)[C@H](O)[C@@H]1O]

Acetonitrile (2.5 mL) was added to a mixture of Intermediate 4 (150 mg, 0.453 mmol), Intermediate 42 (179 mg, 0.453 mmol), and magnesium chloride (43 mg, 0.453 mmol) at RT. The mixture was heated to 50° C. for 5 min, and N,N-diisopropylethylamine (0.197 mL, 0.453 mmol) was added. After 22 h, the reaction mixture was allowed to cool to RT, and concentrated aqueous hydrochloric acid solution (0.5 mL) was added dropwise. After 1 h, the reaction mixture was diluted with ethyl acetate (100 mL) and the resulting mixture was washed with saturated aqueous sodium carbonate solution (50 mL) and brine (50 mL). The organic layer was dried over anhydrous sodium sulfate and concentrated under reduced pressure. The crude residue was subjected to silica gel chromatography eluting with 0-100% ethyl acetate in hexanes to afford the product. ¹H NMR (400 MHz, methanol-d₄) δ 7.78 (s, 0.7H), 7.73 (s, 0.3H), 7.41-7.22 (m, 5H), 6.88-6.79 (m, 1H), 6.76-6.67 (m, 1H), 5.56-5.43 (m, 1H), 5.09-4.93 (m, 2H), 4.69-4.18 (m, 4H), 3.92-3.72 (m, 1H), 3.61 (s, 0.9H), 3.60 (s, 2.1H), 1.31-1.22 (m, 3H). ³¹P NMR (162 MHz, methanol-d₄) δ 7.88 (s), 7.81 (s). LCMS: MS m/z=547.06 [M+1], t_{R}=1.04 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.381 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

### Example 77. Isopropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

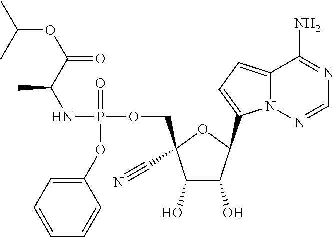 [SMILES: CC(C)OC(=O)[C@H](C)NP(=O)(OC[C@@]1(C#N)O[C@@H](c2ccc3c(N)ncnn23)[C@H](O)[C@@H]1O)Oc1ccccc1]

Intermediate 1 (0.149 g, 0.512 mmol) taken up in anhydrous THF and concentrated. The resulting residue was placed under high vacuum for 1.5 hours. The residue was then dissolved in NMP (4 mL) and then THF (1 mL) was added. This solution was cooled in an ice bath and a 1 M solution of tert-BuMgCl in THF (0.767 mL, 0.767 mmol) was added, causing a white precipitate to form. After 5 minutes the cold bath was removed, the mixture was sonicated to disperse the precipitate solids, and the reaction was stirred at room temperature for 10 minutes. A solution of intermediate isopropyl ((4-nitrophenoxy)(phenoxy)phosphoryl)-L-alaninate (0.251 g, 0.614 mmol; WO2011123668) in THF (0.9 mL) was added. The reaction was stirred at room temperature and progress was monitored by LC/MS. After 1 hour 45 minutes the reaction was cooled in an ice bath and quenched by the addition of glacial AcOH (0.25 mL). The ice bath was removed and stirring was continued for 5 minutes at room temperature. Volatiles were removed by evaporation and the product was isolated from the residue by HPLC. ¹H NMR (400 MHz, Methanol-d₄, chemical shift with asterisk (*) denotes shift of associated proton(s) on the 2^{nd} isomer present) δ 7.81 (s, 0.41H), 7.79* (s, 0.59H), 7.36-7.12 (m, 5H), 6.85 (m, 1H), 6.74 (m, 1H), 5.50 (m, 1H), 4.97-4.85 (m, 1H), 4.63 (m, 1H), 4.54-4.32 (m, 3H), 3.85 (m, 1H), 1.25 (d, J=7.1 Hz, 2H), 1.20* (d, J=6.3 Hz, 4H), 1.16 (t, J=6.3 Hz, 3H). ³¹P NMR (162 MHz, Methanol-d₄) δ 3.30 (s). MS m/z=561.03 [M+1].

Resolution of the Sp and Rp diastereomers. The product was purified via chiral preparatory HPLC (Chiralpak IA, Heptane 70% Ethanol 30%).

Second Eluting Diastereomer: Example 1.

### Example 78. isopropyl ((R)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

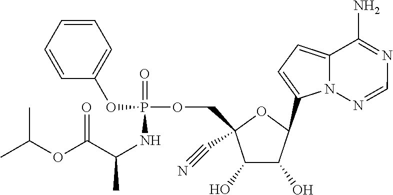 [SMILES: CC(C)OC(=O)[C@H](C)N[P@@](=O)(OC[C@@]1(C#N)O[C@@H](c2ccc3c(N)ncnn23)[C@H](O)[C@@H]1O)Oc1ccccc1]

First Eluting Diastereomer of Example: ¹H NMR (400 MHz, methanol-d₄) δ 7.81 (s, 1H), 7.38-7.24 (m, 2H), 7.22-7.12 (m, 3H), 6.87 (d, J=4.5 Hz, 1H), 6.75 (d, J=4.5 Hz, 1H), 5.53 (d, J=5.0 Hz, 1H), 4.96 (heptet, J=6.3 Hz, 1H), 4.65 (t, J=5.3 Hz, 1H), 4.56-4.46 (m, 2H), 4.38 (dd, J=10.9, 5.2 Hz, 1H), 3.92-3.80 (m, 1H), 1.27 (br d, J=7.1 Hz, 3H), 1.22 (d, J=6.3 Hz, 6H). LCMS: MS m/z=560.96 [M+1], t_{R}=1.48 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.54 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=4.95 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 79. 2-ethylbutyl N2-((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-N6-((benzyloxy)carbonyl)-L-lysinate

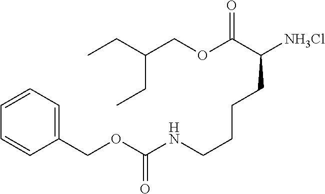

2-ethylbutyl (S)-6-(((benzyloxy)carbonyl)amino)-2-(chloro-15-azanyl)hexanoate. 4 N hydrochloric acid (5 mL) was added to a solution of N6-((benzyloxy)carbonyl)-L-lysine (1 g, 4 mmol) in 2-ethyl-butanol (10 mL) and the resulting mixture was heated to 70° C. After 3 h, the reaction mixture was concentrated under reduced pressure at 70° C. The crude solid residue was taken up into hexanes (150 mL) and was stirred for 4 h. The resulting solid was collected by vacuum filtration to afford the product. ¹H NMR (400 MHz, methanol-d₄) δ 7.38-7.25 (m, 5H), 5.06 (s, 2H), 4.26-4.12 (m, 2H), 4.03 (t, J=6.3 Hz, 1H), 3.13 (t, J=6.7 Hz, 2H), 2.01-1.80 (m, 2H), 1.62-1.32 (m, 8H), 0.92 (t, J=7.5 Hz, 6H).

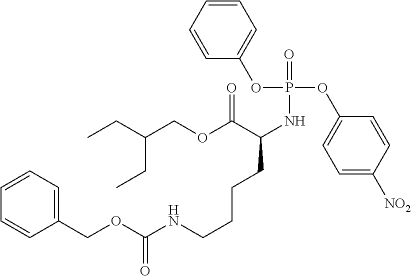 [SMILES: CCC(CC)COC(=O)[C@H](CCCCNC(=O)OCc1ccccc1)NP(=O)(Oc1ccccc1)Oc1ccc([N+](=O)[O-])cc1]

2-ethylbutyl N6-((benzyloxy)carbonyl)-N2-((4-nitrophenoxy)(phenoxy)phosphoryl)-L-lysinate. To a solution of 2-ethylbutyl (S)-6-(((benzyloxy)carbonyl)amino)-2-(chloro-15-azanyl)hexanoate (1.3 g, 3.57 mmol) and phenyl dichlorophosphate (0.753 mL, 3.57 mmol) in dichloromethane (23 mL) was added triethylamine (0.422 mL, 7.14 mmol) at 0° C. under and argon atmosphere. The resulting mixture was allowed to warm to RT and was stirred for 1.5 h. 4-Nitrophenol (496 mg, 3.57 mmol) and triethylamine (0.5 mL, 3.57 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the product (˜1:0.8 diastereomeric mixture). ¹H NMR (400 MHz, chloroform-d) δ 8.27-8.13 (m, 2H), 7.44-7.12 (m, 12H), 5.08 (br s, 2H), 4.72 (br s, 1H), 4.09-3.97 (m, 3H), 3.92-3.72 (m, 1H), 3.18-2.99 (m, 2H), 1.82-1.19 (m, 11H), 0.86 (br t, J=7.4 Hz, 6H). ³¹P NMR (162 MHz, chloroform-d₁) δ −2.49 (s), −2.76 (s). MS m/z=641.97 [M+1].

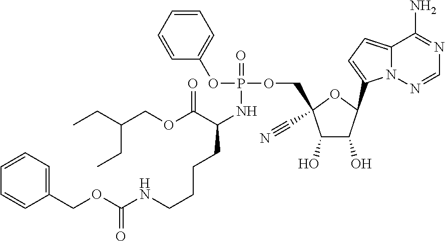 [SMILES: CCC(CC)COC(=O)[C@H](CCCCNC(=O)OCc1ccccc1)NP(=O)(OC[C@@]1(C#N)O[C@@H](c2ccc3c(N)ncnn23)[C@H](O)[C@@H]1O)Oc1ccccc1]

To a mixture of Intermediate 4 (43 mg, 0.130 mmol), 2-ethylbutyl N6-((benzyloxy)carbonyl)-N2-((4-nitrophenoxy)(phenoxy)phosphoryl)-L-lysinate (83.3 mg, 0.130 mmol), and magnesium chloride (12.4 mg, 0.106 mmol) was added acetonitrile (1 mL) at RT. The resulting suspension was warmed to 50° C., and was allowed to stir for 5 min. N,N-Diisopropylethylamine (0.057 mL, 0.324 mmol) was then added and the resulting mixture was stirred at 50° C. for 1 h. The reaction mixture was then allowed to cool to RT, and concentrated aqueous hydrochloric acid solution (12 M, 0.151 mL) was added. After 1 h, the reaction mixture was diluted with saturated aqueous sodium carbonate solutions (20 mL) and ethyl acetate (20 mL). The layers were split and the organic layer was washed with brine (20 mL), was dried over anhydrous sodium sulfate, and was concentrated under reduced pressure. The crude residue purified via SiO₂ column chromatography (40 g SiO₂ Combiflash HP Gold Column, 0-100% ethyl acetate/hexanes) to afford the product. ¹H NMR (400 MHz, methanol-d₄) δ 7.80 (s, 0.4H), 7.77 (s, 0.6H), 7.35-7.09 (m, 10H), 6.88-6.82 (m, 1H), 6.75 (d, J=4.6 Hz, 0.4H), 6.71 (d, J=4.5 Hz, 0.6H), 5.53-5.48 (m, 1H), 5.04 (br s, 2H), 4.65-4.53 (m, 1H), 4.51-4.28 (m, 3H), 4.07-3.76 (m, 3H), 3.08-2.94 (m, 2H), 1.74-1.13 (m, 11H), 0.89-0.78 (m, 6H). ³¹P NMR (162 MHz, methanol-d₄) δ 3.66 (s), 3.27 (s). MS m/z=794.51 [M+1].

### Example 80. (2S)-isopropyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-3-phenylpropanoate

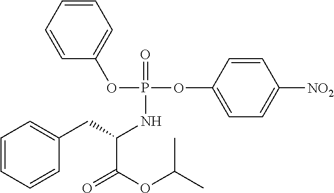 [SMILES: CC(C)OC(=O)[C@H](Cc1ccccc1)NP(=O)(Oc1ccccc1)Oc1ccc([N+](=O)[O-])cc1]

(2S)-isopropyl 2-(((4-nitrophenoxy)(phenoxy)phosphoryl)amino)-3-phenylpropanoate. Phenyl dichlorophosphate (718 μL, 4.8 mmol) was dissolved in anhydrous dichloromethane (20 mL) and stirred under atmosphere nitrogen in an ice bath. Phenylalanine isopropyl ester hydrochloride (1 g, 4.1 mmol) was added in one portion. Triethylamine (736 μL, 5.3 mmol) was added dropwise and stirred for 30 min. More Triethylamine (736 μL, 5.3 mmol) was added dropwise and stirred for 30 mins. Additional Triethylamine (736 μL, 5.3 mmol) was added dropwise and stirred for 15 mins. p-Nitrophenol (600 mg, 4.32 mmol) was added and ice bath was removed. The reaction mixture was then stirred for 2 h. More p-nitrophenol (50 mg) and triethylamine (736 μL, 5.3 mmol) were added and stirred for 1 h. The reaction mixture was concentrated under reduced pressure, diluted with ethyl acetate (50 mL) and washed with 5% aqueous citric acid solution (20 mL) twice, followed with brine (20 mL). The organic layer was dried over anhydrous sodium sulfate and concentrated under reduced pressure. The crude product was purified via SiO₂ column chromatography (24 g SiO₂ Combiflash HP Gold Column 0-15% ethyl acetate/hexanes) to afford the product. ¹H NMR (400 MHz, Chloroform-d) δ 8.17 (t, J=9.0 Hz, 2H), 7.38-7.13 (m, 10H), 7.13-7.02 (m, 2H), 4.95 (pd, J=6.3, 3.9 Hz, 1H), 4.31 (ddq, J=10.6, 9.2, 6.2 Hz, 1H), 3.69 (td, J=10.9, 4.6 Hz, 1H), 3.02 (dd, J=6.1, 1.8 Hz, 2H), 1.21-1.08 (m, 6H). ³¹P NMR (162 MHz, Chloroform-d) δ −2.97, −2.98. MS m/z=485.0 [M+1], 483.2 [M−1].

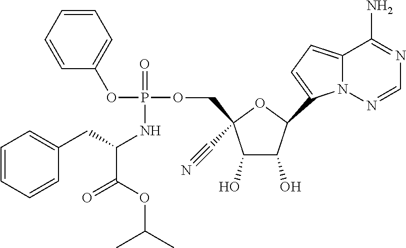 [SMILES: CC(C)OC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

Intermediate 2 (50 mg, 0.116 mmol) and (2S)-isopropyl 2-(((4-nitrophenoxy)(phenoxy)phosphoryl)amino)-3-phenylpropanoate (67 mg, 0.139 mmol) were dissolved in anhydrous tetrahydrofuran (3 mL). Magnesium chloride (17 mg, 0.174 mmol) was added in one portion. Reaction was warmed to 50° C. and stirred for 20 mins. N,N-Diisopropylethylamine (50 μL, 0.29 mmol) was added, and the reaction was stirred for 3 h at 50° C. Reaction was heated and stirred at 60° C. for 17 h. The reaction mixture was cooled to room temperature. 4 N hydrogen chloride in 1,4-dioxane (5 mL) along with methanol (500 μL) were added to the reaction mixture which was the stirred for 2 h. Water (1 mL) was added and stirred for 1 h. The mixture was diluted with ethyl acetate (30 mL) and cooled in an ice bath. 1 N aqueous sodium hydroxide solution was added slowly to give pH of 10. The organic layer was collected and washed with 5% aqueous sodium carbonate solution (20 mL) twice and then brine (20 mL). Organic layer was dried over anhydrous sodium sulfate and concentrated under reduced pressure. The crude product was purified via SiO₂ column chromatography (4 g SiO₂ Combiflash HP Gold Column 0-3-10% methanol/dichloromethane). Fractions with the desired product were combined and concentrated under reduced pressure. The residue was purified with Prep HPLC (Phenomenex Gemini C₁₈ column, 0-100% acetonitrile/water with 0.1% trifluoroacetic acid as modifier). Fractions containing the product were combined and diluted with ethyl acetate (30 mL) and washed with saturated aqueous sodium bicarbonate solution (30 mL) and then brine (10 mL). Organic layer was dried over anhydrous sodium sulfate and concentrated under reduced pressure. Residue was dissolved in acetonitrile and water and freeze dried to give the product. ¹H NMR (400 MHz, Chloroform-d) δ 7.66 (s, 1H), 7.28-7.01 (m, 10H), 6.53 (dd, J=11.3, 5.1 Hz, 2H), 5.55-5.38 (m, 1H), 4.91 (m, 1H), 4.43 (m, 2H), 4.34-4.01 (m, 3H), 3.10-2.81 (m, 2H), 1.12 (m, 6H). ³¹P NMR (162 MHz, Chloroform-d) δ 2.90, 2.73 MS m/z=637.1 [M+1], 635.0 [M−1].

### Example 81. (2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-3-phenylpropanoate

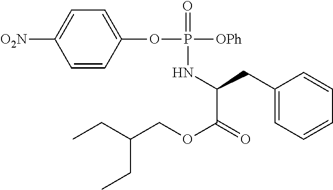 [SMILES: CCC(CC)COC(=O)[C@H](Cc1ccccc1)NP(=O)(Oc1ccccc1)Oc1ccc([N+](=O)[O-])cc1]

(2S)-2-ethylbutyl 2-(((4-nitrophenoxy)(phenoxy)phosphoryl)amino)-3-phenylpropanoate. Phenyl dichlorophosphate (354 μL, 2.38 mmol) was dissolved in anhydrous dichloromethane (24 mL) and stirred under atmosphere argon in an ice bath. L-Phenylalanine 2-ethylbutyl ester hydrochloride (680 mg, 2.38 mmol) was added in one portion. Triethylamine (730 μL, 5.24 mmol) was added dropwise. The reaction mixture was stirred for 2 h. More triethylamine (365 uL, 2.62 mmol) was added dropwise and stirred for 60 mins. p-Nitrophenol (265 mg, 1.9 mmol) was added. The reaction mixture was stirred for 2 h and was diluted with dichloromethane (30 mL) and washed with 5% aqueous sodium carbonate solution (20 mL) twice. Dried organic layer over anhydrous sodium sulfate and concentrated under reduced pressure. The crude product was purified via SiO₂ column chromatography (24 g SiO₂ Combiflash HP Gold Column 0-20% ethyl acetate/hexanes) to afford the product. ¹H NMR (400 MHz, DMSO-d₆) δ 8.23-8.14 (m, 2H), 7.33 (t, J=7.8 Hz, 2H), 7.28-7.10 (m, 7H), 7.07 (m, 2H), 6.85 (m, 1H), 4.15-3.97 (m, 1H), 3.90-3.75 (m, 2H), 2.97 (m, 1H), 2.79 (m, 1H), 1.39-1.25 (m, 1H), 1.17 (dtd, J=8.8, 7.2, 3.6 Hz, 4H), 0.73 (tt, J=7.6, 1.7 Hz, 6H).

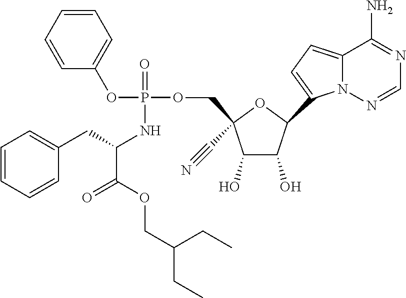 [SMILES: CCC(CC)COC(=O)[C@H](Cc1ccccc1)NP(=O)(OC[C@@]1(C#N)O[C@@H](c2ccc3c(N)ncnn23)[C@H](O)[C@@H]1O)Oc1ccccc1]

Intermediate 2 (50 mg, 0.116 mmol) and (2S)-2-ethylbutyl 2-(((4-nitrophenoxy)(phenoxy)phosphoryl)amino)-3-phenylpropanoate (73 mg, 0.139 mmol) were dissolved in anhydrous tetrahydrofuran (3 mL). Magnesium chloride (17 mg, 0.174 mmol) was added in one portion. Reaction was warmed to 50° C. and stirred for 20 mins. N,N-Diisopropylethylamine (50 μL, 0.29 mmol) was added, and the reaction was stirred for 3 h at 50° C. Reaction was heated and stirred at 60° C. for 17 h. The reaction mixture was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL), added aqueous trifluoroacetic acid solution (v/v 1:1, 2 mL) and stirred for 20 h. The reaction mixture was diluted with ethyl acetate (30 mL) and cooled in an ice bath. Aqueous potassium hydroxide solution was added dropwise to give pH of 10. The organic layer was collected and washed with saturated aqueous sodium bicarbonate solution (10 mL) and then brine (10 mL). The organic layer was dried over anhydrous sodium sulfate and concentrated under reduced pressure. The crude product was purified via SiO₂ column chromatography (4 g SiO₂ Combiflash HP Gold Column 0-3-5% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.78 (m, 1H), 7.33-6.97 (m, 10H), 6.84 (dd, J=4.5, 2.9 Hz, 1H), 6.70 (dd, J=4.5, 3.0 Hz, 1H), 5.54-5.44 (m, 1H), 4.59-4.50 (m, 1H), 4.37 (m, 1H), 4.26-4.02 (m, 3H), 4.02-3.80 (m, 3H), 2.99 (m, 1H), 2.85 (m, 1H), 1.42-1.29 (m, 1H), 1.29-1.13 (m, 4H), 0.80 (m, 6H). ³¹P NMR (162 MHz, Methanol-d₄) δ 3.21, 2.70. MS m/z=679.1 [M+1], 677.1 [M−1].

### Example 82. (2R)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

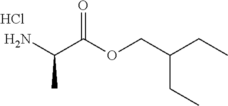

(R)-2-ethylbutyl 2-aminopropanoate hydrochloride. D-Alanine (891 mg, 10 mmol) was mixed with 2-ethyl-1butanol (20 mL). Trimethylsilyl chloride (3.8 mL, 30 mmol) was added dropwise and stirred for 30 min. More Trimethylsilyl chloride (3.8 mL, 30 mmol) was added dropwise. Reaction mixture was heated to 50° C. and stirred for 2 h. More Trimethylsilyl chloride (3.8 mL, 30 mmol) was added dropwise. The reaction mixture was heated to 75° C. and stirred for 18 h. The reaction mixture was concentrated under reduced pressure at 80° C. The residue was cooled in an ice bath. Hexanes (100 mL) was added and stirred for 1 h. The solids were collected and washed with hexanes (100 mL) and dried under high vacuum to give the product. ¹H NMR (400 MHz, DMSO-d₆) δ 8.42 (s, 3H), 4.18-3.96 (m, 3H), 1.58-1.43 (m, 1H), 1.39 (d, J=7.2 Hz, 3H), 1.31 (ddq, J=10.2, 7.3, 3.7 Hz, 4H), 0.85 (t, J=7.4 Hz, 6H).

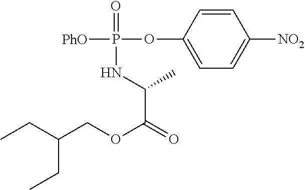 [SMILES: CCC(CC)COC(=O)[C@@H](C)NP(=O)(Oc1ccccc1)Oc1ccc([N+](=O)[O-])cc1]

(2R)-2-ethylbutyl 2-(((4-nitrophenoxy)(phenoxy)phosphoryl)amino)propanoate. Phenyl dichlorophosphate (354 μL, 2.38 mmol) was dissolved in anhydrous dichloromethane (24 mL) and stirred under atmosphere nitrogen in an ice bath. D-Alanine 2-ethylbutylester hydrochloride (500 mg, 2.38 mmol) was added in one portion. Triethylamine (730 μL, 5.24 mmol) was added dropwise and stirred for 2 h. More Triethylamine (365 μL, 2.62 mmol) was added dropwise and stirred for 30 min. p-Nitrophenol (265 mg, 1.9 mmol) was added and ice bath was removed. Reaction was then stirred for 2 hrs. The reaction mixture was diluted with dichloromethane (20 mL) and washed with 5% aqueous sodium carbonate solution (2×20 mL). The organic layer was dried over anhydrous sodium sulfate and concentrated under reduced pressure. The crude product was purified via SiO₂ column chromatography (24 g SiO₂ Combiflash HP Gold Column 0-15% ethyl acetate/hexanes) to afford the product. ¹H NMR (400 MHz, DMSO-d₆) δ 8.32-8.22 (m, 2H), 7.53-7.32 (m, 4H), 7.29-7.15 (m, 3H), 6.75-6.60 (m, 1H), 4.08-3.93 (m, 1H), 3.93-3.82 (m, 2H), 1.38 (m, 1H), 1.31-1.17 (m, 7H), 0.78 (t, J=7.5 Hz, 6H). ³¹P NMR (162 MHz, DMSO-d₆) δ −1.26, −1.48. MS m/z=451.0 [M+1], 449.1 [M−1].

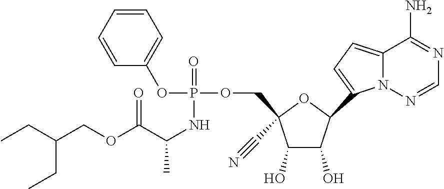 [SMILES: CCC(CC)COC(=O)[C@@H](C)NP(=O)(OC[C@@]1(C#N)O[C@@H](c2ccc3c(N)ncnn23)[C@H](O)[C@@H]1O)Oc1ccccc1]

(2R)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. Intermediate 2 (50 mg, 0.116 mmol) and (2R)-2-ethylbutyl 2-(((4-nitrophenoxy)(phenoxy)phosphoryl)amino)propanoate (63 mg, 0.139 mmol) were dissolved in anhydrous tetrahydrofuran (3 mL). Magnesium chloride (17 mg, 0.174 mmol) was added in one portion. Reaction was warmed to 50° C. and stirred for 20 min. N,N-Diisopropylethylamine (50 μL, 0.29 mmol) was added, and the reaction was stirred for 3 h at 50° C. Reaction was heated and stirred at 60° C. for 17 h. The reaction mixture was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL), added aqueous trifluoroacetic acid solution (v/v 1:1, 2 mL) and stirred for 20 h. The reaction mixture was diluted with ethyl acetate (30 mL) and cooled in an ice bath. Aqueous potassium hydroxide solution was added dropwise to give pH of 10. Organic layer was collected and washed with saturated aqueous sodium bicarbonate solution (10 mL) and then brine (10 mL). Organic layer was dried over anhydrous sodium sulfate and concentrated under reduced pressure. The crude product was purified via SiO₂ column chromatography (4 g SiO₂ Combiflash HP Gold Column 0-3-5% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.38-7.23 (m, 2H), 7.23-7.09 (m, 3H), 6.85 (dd, J=5.5, 4.5 Hz, 1H), 6.73 (dd, J=15.4, 4.5 Hz, 1H), 5.55-5.46 (m, 1H), 4.61 (dt, J=20.1, 5.3 Hz, 1H), 4.53-4.25 (m, 3H), 4.14-3.83 (m, 3H), 1.50-1.38 (m, 1H), 1.37-1.17 (m, 7H), 0.85 (tdd, J=7.5, 3.9, 1.6 Hz, 6H). ³¹P NMR (162 MHz, Methanol-d₄) δ 3.37, 3.06. MS m/z=603.1 [M+1], 601.1 [M−1].

Separation of the (S) and (R) Diastereomers. The product was purified via chiral preparatory HPLC (Chiralpak IC, 150×4.6 mm, Heptane 70% Ethanol 30%) to afford the diastereomers:

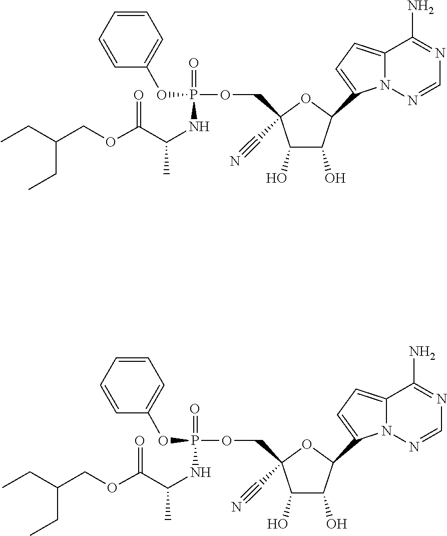 [SMILES: CCC(CC)COC(=O)[C@@H](C)N[P@@](=O)(OC[C@@]1(C#N)O[C@@H](c2ccc3c(N)ncnn23)[C@H](O)[C@@H]1O)Oc1ccccc1 ; CCC(CC)COC(=O)[C@@H](C)N[P@](=O)(OC[C@@]1(C#N)O[C@@H](c2ccc3c(N)ncnn23)[C@H](O)[C@@H]1O)Oc1ccccc1]

### Example 83. First Eluting Diastereomer

¹H NMR (400 MHz, Methanol-d₄) δ 7.81 (s, 1H), 7.38-7.28 (m, 2H), 7.23-7.13 (m, 3H), 6.84 (d, J=4.5 Hz, 1H), 6.75 (d, J=4.5 Hz, 1H), 5.51 (d, J=5.1 Hz, 1H), 4.64 (t, J=5.3 Hz, 1H), 4.49 (d, J=5.6 Hz, 1H), 4.48-4.33 (m, 2H), 4.05-3.88 (m, 3H), 1.50-1.40 (m, 1H), 1.37-1.24 (m, 7H), 0.95-0.80 (m, 6H). ³¹P NMR (162 MHz, Methanol-d₄) δ 3.39.

### Example 84. Second Eluting Diastereomer

¹H NMR (400 MHz, Methanol-d₄) δ 7.78 (s, 1H), 7.34-7.25 (m, 2H), 7.23-7.11 (m, 3H), 6.86 (d, J=4.5 Hz, 1H), 6.71 (d, J=4.5 Hz, 1H), 5.49 (d, J=5.0 Hz, 1H), 4.58 (t, J=5.3 Hz, 1H), 4.44 (m, 2H), 4.31 (m, 1H), 4.04 (m, 1H), 3.97-3.85 (m, 2H), 1.44 (dt, J=12.4, 6.2 Hz, 1H), 1.36-1.24 (m, 7H), 0.86 (m, 6H). ³¹P NMR (162 MHz, Methanol-d₄) δ 3.06

### Example 85. (2S)-ethyl 6-acetamido-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)hexanoate

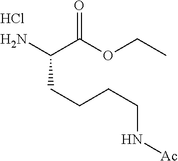

(S)-ethyl 6-acetamido-2-aminohexanoate hydrochloride. (S)-6-acetamido-2-aminohexanoic acid (1.88 g, 10 mmol) was mixed with ethanol (25 mL). Trimethylsilyl chloride (6.3 mL, 50 mmol) was added dropwise and stirred for 30 min. Reaction mixture was heated to 80° C. and stirred for 20 h. The reaction mixture was concentrated under reduced pressure to give an oil which was mixed with hexane (100 mL) and stirred for 30 min. Solvent was decanted off. More hexanes (100 mL) was added and stirred for 30 mins. Solvent was decanted off and the resulting oil was dried under high vacuum to give title compound. ¹H NMR (400 MHz, DMSO-d₆) δ 8.59 (bs, 3H), 7.94 (s, 1H), 4.18 (m, 2H), 3.92 (d, J=5.8 Hz, 1H), 2.98 (d, J=6.1 Hz, 2H), 1.77 (m, 5H), 1.45-1.30 (m, 2H), 1.21 (t, J=7.1 Hz, 3H).

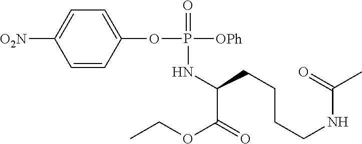 [SMILES: CCOC(=O)[C@H](CCCCNC(C)=O)NP(=O)(Oc1ccccc1)Oc1ccc([N+](=O)[O-])cc1]

(2S)-ethyl 6-acetamido-2-(((4-nitrophenoxy)(phenoxy)phosphoryl)amino)hexanoate. Phenyl dichlorophosphate (354 μL, 2.38 mmol) was dissolved in anhydrous dichloromethane (24 mL) and stirred under atmosphere nitrogen in an ice bath. (S)-ethyl 6-acetamido-2-aminohexanoate hydrochloride (601 mg, 2.38 mmol) was added in one portion. Triethylamine (730 μL, 5.3 mmol) was added dropwise and stirred for 2 h. More Triethylamine (365 μL, 2.62 mmol) was added dropwise and stirred for 60 mins. p-Nitrophenol (265 mg, 1.9 mmol) was added and ice bath was removed. Reaction was then stirred for 2 h. The reaction was diluted with dichloromethane (20 mL) and washed with 5% aqueous sodium carbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-100% ethyl acetate/hexanes) to afford the product. ¹H NMR (400 MHz, DMSO-d₆) δ 8.33-8.22 (m, 2H), 7.70 (t, J=5.7 Hz, 1H), 7.53-7.33 (m, 4H), 7.29-7.14 (m, 3H), 6.63 (dt, J=13.4, 10.6 Hz, 1H), 3.99 (qd, J=7.1, 2.1 Hz, 2H), 3.78 (qt, J=9.7, 5.4 Hz, 1H), 2.87 (qd, J=6.8, 3.4 Hz, 2H), 1.74 (s, 3H), 1.51 (m, 2H), 1.34-1.00 (m, 7H). MS m/z=494.1 [M+1], 492.2 [M−1].

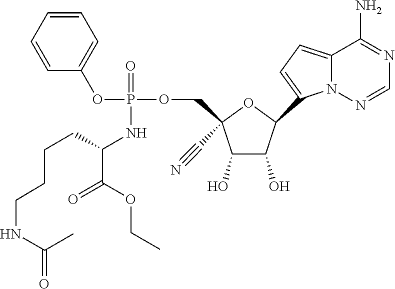 [SMILES: CCOC(=O)[C@H](CCCCNC(C)=O)NP(=O)(OC[C@@]1(C#N)O[C@@H](c2ccc3c(N)ncnn23)[C@H](O)[C@@H]1O)Oc1ccccc1]

(2S)-ethyl 6-acetamido-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)hexanoate. Intermediate 2 (50 mg, 0.116 mmol) and (2S)-ethyl 6-acetamido-2-(((4-nitrophenoxy)(phenoxy)phosphoryl)amino)hexanoate (69 mg, 0.139 mmol) were dissolved in anhydrous tetrahydrofuran (3 mL). Magnesium chloride (17 mg, 0.174 mmol) was added in one portion. Reaction was warmed to 50° C. and stirred for 20 min. N,N-Diisopropylethylamine (50 μL, 0.29 mmol) was added, and the reaction was stirred for 3 h at 50° C. Reaction was heated and stirred at 60° C. for 17 h. The reaction was cooled to room temperature, diluted with ethyl acetate (30 mL) and washed with 5% aqueous sodium carbonate solution (3×20 mL) and then brine (20 mL). Organic layer was dried over anhydrous sodium sulfate and concentrated under reduced pressure. Residue was dissolved in acetonitrile (2 mL), added aqueous trifluoroacetic acid solution (v/v 1:1, 2 mL) and stirred for 20 h. The reaction was diluted with ethyl acetate (30 mL) and cooled in an ice bath. Aqueous potassium hydroxide solution was added dropwise to give pH of 10. Organic layer was collected and washed with saturated aqueous sodium bicarbonate solution (10 mL) and then brine (10 mL). Organic layer was dried over anhydrous sodium sulfate and concentrated under reduced pressure. The crude product was purified via SiO₂ column chromatography (4 g SiO₂ Combiflash HP Gold Column 0-3-10% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.38-7.26 (m, 2H), 7.26-7.10 (m, 3H), 6.85 (dd, J=4.5, 3.3 Hz, 1H), 6.74 (dd, J=4.5, 3.0 Hz, 1H), 5.54-5.45 (m, 1H), 4.63 (td, J=5.4, 4.4 Hz, 1H), 4.54-4.27 (m, 3H), 4.08-3.93 (m, 2H), 3.80 (m, 1H), 3.05 (tq, J=6.0, 1.8 Hz, 2H), 1.90 (m, 3H), 1.74-1.62 (m, 1H), 1.62-1.48 (m, 1H), 1.47-1.25 (m, 4H), 1.16 (t, J=7.1 Hz, 3H). ³¹P NMR (162 MHz, Methanol-d₄) δ 3.54, 3.44. MS m/z=646.1 [M+1], 644.1 [M−1].

### Example 86. (2R)-isopropyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

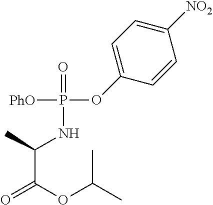 [SMILES: CC(C)OC(=O)[C@@H](C)NP(=O)(Oc1ccccc1)Oc1ccc([N+](=O)[O-])cc1]

isopropyl ((4-nitrophenoxy)(phenoxy)phosphoryl)-D-alaninate. D-alanine isopropylester-HCl (1.21 g, 7.22 mmol) was suspended in methylene chloride (15 mL) and the resulting mixture was cooled to −78° C. Phenyl dichlorophosphate (1.08 mL, 7.22 mmol) was added. Triethylamine (2.0 mL, 14.44 mmol) was then added over 30 min at −78° C. 4-nitrophenol (1004 mg, 7.22 mmol) was then added in one portion. Then triethylamine (1.0 mL, 7.22 mmol) was added over 30 min at −78° C. The resulting mixture was stirred for 30 min at −78° C., and was then allowed to warm to RT and was washed with water (2×) and saturate sodium bicarbonate solution, dried over sodium sulfate, and concentrated in vacuo. The residue was purified by silica gel column chromatography (ethyl acetate 0 to 30% in hexanes) to afford the product. ¹H NMR (400 MHz, Chloroform-d) δ 8.32-8.14 (m, 2H), 7.44-7.31 (m, 4H), 7.31-7.15 (m, 3H), 5.01 (pd, J=6.3, 1.0 Hz, 1H), 4.22-3.99 (m, 1H), 3.89 (dd, J=12.2, 9.1 Hz, 1H), 1.48-1.32 (m, 3H), 1.33-1.11 (m, 6H); ³¹P NMR (162 MHz, Chloroform-d) δ −3.00, −3.03; MS m/z 409 (M+1)+.

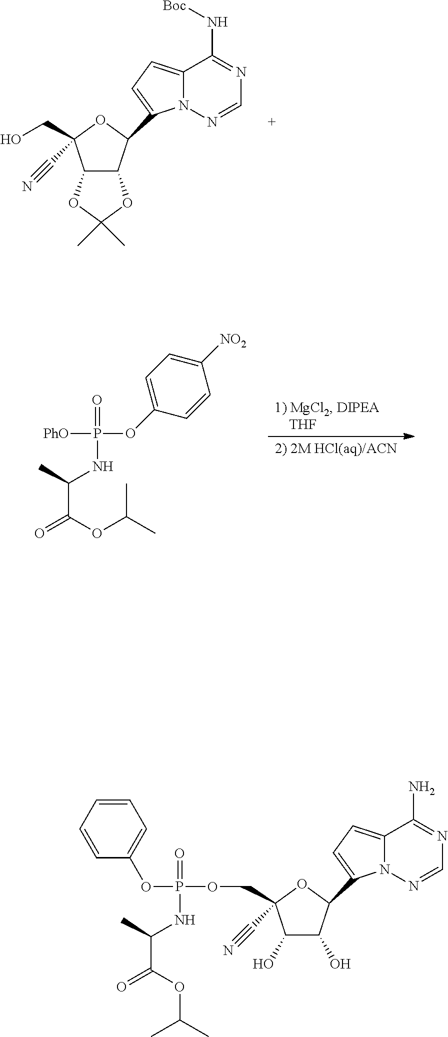

(2R)-isopropyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. Intermediate 2 (50 mg, 0.116 mmol) and isopropyl ((4-nitrophenoxy)(phenoxy)phosphoryl)-D-alaninate (57 mg, 0.139 mmol) were dissolved in anhydrous tetrahydrofuran (3 mL). Magnesium chloride (17 mg, 0.174 mmol) was added in one portion. Reaction was warmed to 60° C. and stirred for 20 min. N,N-Diisopropylethylamine (50 μL, 0.29 mmol) was added, and the reaction was stirred at 60° C. for 17 h. The reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (330 μL) was added dropwise and stirred for 20 h. The reaction was diluted with ethyl acetate (30 mL) and cooled in an ice bath. 1 N sodium hydroxide solution was added dropwise to give pH of 10. Organic layer was collected and washed with saturated aqueous sodium bicarbonate solution (10 mL) and then brine (10 mL). Organic layer was dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d4) δ 7.79 (m, 1H), 7.30 (m, 2H), 7.23-7.10 (m, 3H), 6.84 (t, J=4.7 Hz, 1H), 6.73 (dd, J=13.9, 4.5 Hz, 1H), 5.53-5.46 (m, 1H), 4.93 (m, 1H), 4.62 (m, 1H), 4.52-4.41 (m, 2H), 4.35 (m, 1H), 3.93-3.78 (m, 1H), 1.26-1.14 (m, 9H). ³¹P NMR (162 MHz, Methanol-d₄) δ 3.41, 3.13. MS m/z=561.2 [M+1], 559.2 [M−1].

### Example 87. (2S)-isopropyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

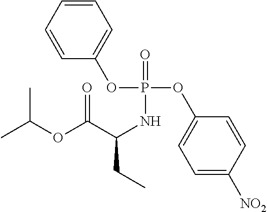 [SMILES: CC[C@H](NP(=O)(Oc1ccccc1)Oc1ccc([N+](=O)[O-])cc1)C(=O)OC(C)C]

(2S)-isopropyl 2-(((4-nitrophenoxy)(phenoxy)phosphoryl)amino)butanoate. (2S)-isopropyl-2-aminobutanoate hydrochloride (1.28 g, 7.05 mmol, 1:1.6 isomeric mixture) was converted to the intermediate by using the same procedure used for Example 88. ¹H NMR (400 MHz, Methanol-d4) δ 8.33-8.23 (m, 2H), 7.55-7.34 (m, 4H), 7.34-7.17 (m, 3H), 5.00-4.87 (m, 1H), 3.84 (m, 1H), 1.86-1.49 (m, 2H), 1.18 (m, 6H), 0.87 (m, 3H). ³¹P NMR (162 MHz, Methanol-d4) δ −0.91, −1.10. MS m/z=423 (M+H)⁺.

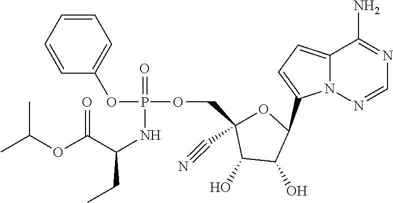 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC(C)C]

(2S)-isopropyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. The product was obtained from intermediate (2S)-isopropyl 2-(((4-nitrophenoxy)(phenoxy)phosphoryl)amino)butanoate (80 mg, 0.19 mmol) and Intermediate 4 (117 mg, 0.28 mmol) using the same procedure used for Example 88. ¹H NMR (400 MHz, Methanol-d4) δ 7.80 (s, 0.7H), 7.79 (s, 0.3H), 7.39-7.25 (m, 2H), 7.25-7.13 (m, 3H), 6.86 (m, 1H), 6.74 (m, 1H), 5.49 (m, 1H), 4.98-4.82 (m, 1H), 4.62 (m, 1H), 4.45 (m, 2H), 4.34 (m 1H), 3.72 (m, 1H), 1.77-1.47 (m, 2H), 1.21-1.12 (m, 6H), 0.83 (m, 3H). ³¹P NMR (162 MHz, Methanol-d4) δ 3.68, 3.65. MS m/z=575 (M+H)⁺.

### Example 88. (2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

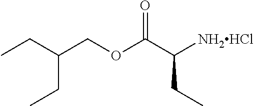

(S)-2-ethylbutyl 2-aminobutanoate hydrochloride. To a mixture of (S)-2-aminobutanoic acid (2.5 g, 24.24 mmol) and 2-ethyl-butan-1-ol (25 mL) was added TMSCl (8.64 mL). The resulting mixture was stirred at 70° C. for 15 h and concentrated in rotary evaporator at 80° C., co-evaporated with toluene several times, dried under high vacuum, to afford the intermediate and used in next reaction.

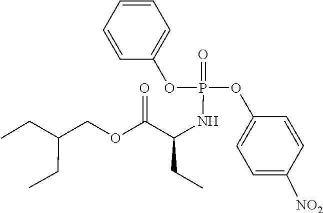 [SMILES: CCC(CC)COC(=O)[C@H](CC)NP(=O)(Oc1ccccc1)Oc1ccc([N+](=O)[O-])cc1]

(2S)-2-ethylbutyl 2-(((4-nitrophenoxy)(phenoxy)phosphoryl)amino)butanoate. Compound (S)-2-ethylbutyl 2-aminobutanoate hydrochloride (660 mg, 2.95 mmol) was suspended in methylene chloride (6 mL), cooled to −78° C., and phenyl dichlorophosphate (0.44 mL, 2.95 mmol) added quickly. Triethylamine (0.82 mL, 5.90 mmol) was added over 30 min at −78° C. and 4-nitrophenol (410 mg, 2.95 mmol) was added in one portion. Then triethylamine (0.41 mL, 2.95 mmol) was added over 30 min at −78° C. The resulting mixture was stirred for 30 min at −78° C., washed with water twice and brine, dried over sodium sulfate, and concentrated in vacuo. The residue was purified by silica gel column chromatography (EtOAc 0 to 30% in hexanes) to give the intermediate. ¹H NMR (400 MHz, Methanol-d4) δ 8.34-8.22 (m, 2H), 7.52-7.32 (m, 4H), 7.31-7.18 (m, 3H), 3.99 (m, 1.1 Hz, 2H), 3.89 (m, 1H), 1.76 (m, 1H), 1.65 (m, 1H), 1.50-1.38 (m, 1H), 1.38-1.25 (m, 4H), 0.97-0.79 (m, 9H). ³¹P NMR (162 MHz, Methanol-d4) δ −0.94, −1.20. MS m/z 465 (M+H)⁺.

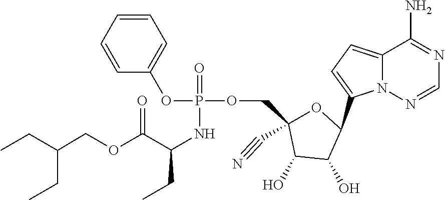 [SMILES: CCC(CC)COC(=O)[C@H](CC)NP(=O)(OC[C@@]1(C#N)O[C@@H](c2ccc3c(N)ncnn23)[C@H](O)[C@@H]1O)Oc1ccccc1]

(2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. To a mixture of Intermediate 4 (89 mg, 0.21 mmol), intermediate (2S)-2-ethylbutyl 2-(((4-nitrophenoxy)(phenoxy)phosphoryl)amino)butanoate (130 mg, 0.28 mmol), and MgCl₂ (30 mg, 0.31 mmol) in DMF (4 mL) was N,N-diisopropylethylamine (0.09 mL, 0.52 mmol) dropwise at room temperature. The resulting mixture was stirred at 50° C. for 4 h, then diluted with EtOAc, washed with water and brine, dried with sodium sulfate, and concentrated in vacuo. The obtained residue was dissolved in ACN (3 mL) and c-HCl (0.5 mL) was added. The resulting mixture was stirred at 50° C. for 2 h and purified by preparative HPLC (Phenominex Synergi 4 u Hydro-RR 80 Å 150×30 mm column, 10-70% acetonitrile/water gradient) to afford the product. ¹H NMR (400 MHz, Methanol-d4) δ 7.79 (s, 0.63H), 7.78 (s, 0.37H), 7.25 (m, 5H), 6.97-6.58 (m, 2H), 5.49 (m, 1H), 4.61 (m, 1H), 4.45 (m, 2H), 4.34 (m, 1H), 3.99 (m, 1H), 3.88 (m, 1H), 3.83-3.73 (m, 1H), 1.77-1.65 (m, 1H), 1.59 (m, 1H), 1.43 (m, 1H), 1.38-1.21 (m, 4H), 0.85 (m, 9H). ³¹P NMR (162 MHz, Methanol-d4) δ 3.62, 3.59. MS m/z 617 (M+H)⁺.

Separation of the (S) and (R) Diastereomers. The product was purified via chiral preparatory HPLC (Chiralpak IA, 150×4.6 mm, Heptane 70% Ethanol 30%) to afford the diastereomers:

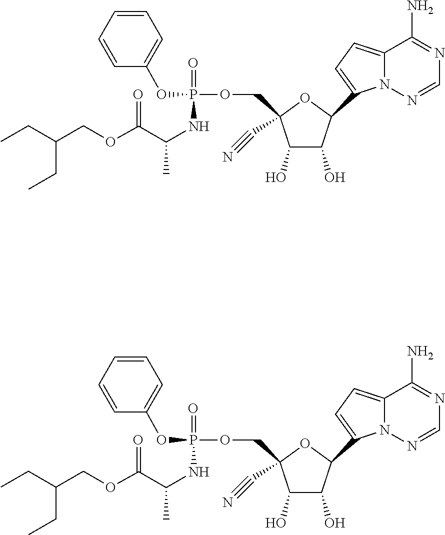 [SMILES: CCC(CC)COC(=O)[C@@H](C)N[P@@](=O)(OC[C@@]1(C#N)O[C@@H](c2ccc3c(N)ncnn23)[C@H](O)[C@@H]1O)Oc1ccccc1 ; CCC(CC)COC(=O)[C@@H](C)N[P@](=O)(OC[C@@]1(C#N)O[C@@H](c2ccc3c(N)ncnn23)[C@H](O)[C@@H]1O)Oc1ccccc1]

### Example 89. First Eluting Diastereomer

¹H NMR (400 MHz, Methanol-d₄) δ 7.78 (s, 1H), 7.28 (m, 2H), 7.20-7.11 (m, 3H), 6.85 (d, J=4.5 Hz, 1H), 6.73 (d, J=4.5 Hz, 1H), 5.50 (d, J=5.1 Hz, 1H), 4.62 (t, J=5.4 Hz, 1H), 4.47 (m, 2H), 4.35 (m, 1H), 4.06-3.93 (m, 2H), 3.78 (m, 1H), 1.80-1.66 (m, 1H), 1.60 (m, 1H), 1.53-1.42 (m, 1H), 1.39-1.22 (m, 4H), 0.94-0.79 (m, 9H). ³¹P NMR (162 MHz, Methanol-d₄) δ 3.59.

### Example 90. Second Eluting Diastereomer

¹H NMR (400 MHz, Methanol-d₄) δ 7.79 (s, 1H), 7.32 (m, 2H), 7.26-7.12 (m, 3H), 6.84 (d, J=4.5 Hz, 1H), 6.74 (d, J=4.5 Hz, 1H), 5.48 (d, J=4.9 Hz, 1H), 4.64-4.58 (m, 1H), 4.46 (d, J=5.6 Hz, 1H), 4.45-4.29 (m, 2H), 4.01-3.83 (m, 2H), 3.82-3.73 (m, 1H), 1.77-1.65 (m, 1H), 1.59 (m, 1H), 1.42 (m, 1H), 1.30 (m, 4H), 0.84 (m, 9H). ³¹P NMR (162 MHz, Methanol-d₄) δ 3.62.

### Example 91. (2S)-2-ethylbutyl 6-amino-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)hexanoate

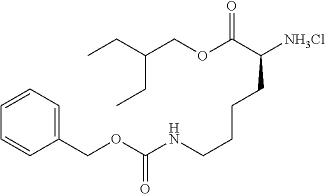

2-ethylbutyl N6-((benzyloxy)carbonyl)-L-lysinate hydrochloride. 4 N hydrochloric acid (5 mL) was added to a solution of N6-((benzyloxy)carbonyl)-L-lysine (1 g, 4 mmol) in 2-ethyl-butanol (10 mL) and the resulting mixture was heated to 70° C. After 3 h, the reaction mixture was concentrated under reduced pressure at 70° C. The crude solid residue was taken up into hexanes (150 mL) and was stirred for 4 h. The resulting white crystalline solid was collected by vacuum filtration to afford the intermediate. ¹H NMR (400 MHz, CD₃OD) δ 7.38-7.25 (m, 5H), 5.06 (s, 2H), 4.26-4.12 (m, 2H), 4.03 (t, J=6.3 Hz, 1H), 3.13 (t, J=6.7 Hz, 2H), 2.01-1.80 (m, 2H), 1.62-1.32 (m, 8H), 0.92 (t, J=7.5 Hz, 6H).

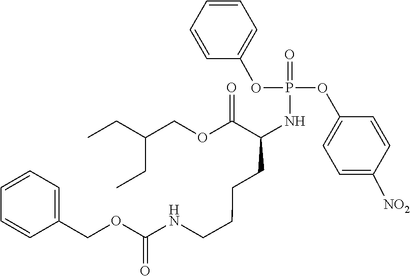 [SMILES: CCC(CC)COC(=O)[C@H](CCCCNC(=O)OCc1ccccc1)NP(=O)(Oc1ccccc1)Oc1ccc([N+](=O)[O-])cc1]

2-ethylbutyl N6-((benzyloxy)carbonyl)-N2-((4-nitrophenoxy)(phenoxy)phosphoryl)-L-lysinate. To a solution of 2-ethylbutyl N6-((benzyloxy)carbonyl)-L-lysinate hydrochloride (1.3 g, 3.57 mmol) and phenyl dichlorophosphate (0.753 mL, 3.57 mmol) in dichloromethane (23 mL) was added triethylamine (0.422 mL, 7.14 mmol) at 0° C. under and argon atmosphere. The resulting mixture was allowed to warm to RT and was stirred for 1.5 h. 4-Nitrophenol (496 mg, 3.57 mmol) and triethylamine (0.5 mL, 3.57 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. MS m/z=641.97 [M+1].

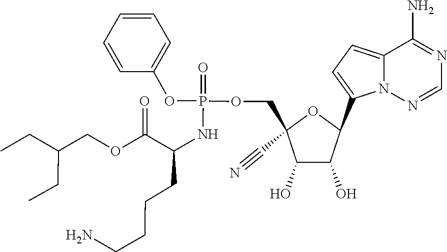 [SMILES: CCC(CC)COC(=O)[C@H](CCCCN)NP(=O)(OC[C@@]1(C#N)O[C@@H](c2ccc3c(N)ncnn23)[C@H](O)[C@@H]1O)Oc1ccccc1]

(2S)-2-ethylbutyl 6-amino-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)hexanoate. To a mixture of Intermediate 4 (43 mg, 0.130 mmol), intermediate 2-ethylbutyl N6-((benzyloxy)carbonyl)-N2-((4-nitrophenoxy)(phenoxy)phosphoryl)-L-lysinate (83.3 mg, 0.130 mmol), and magnesium chloride (12.4 mg, 0.106 mmol) was added acetonitrile (1 mL) at RT. The resulting suspension was warmed to 50° C., and was allowed to stir for 5 min. N,N-Diisopropylethylamine (0.057 mL, 0.324 mmol) was then added and the resulting mixture was stirred at 50° C. for 1 h. The reaction mixture was then allowed to cool to RT, and concentrated aqueous hydrochloric acid solution (12 M, 0.151 mL) was added. After 1 h, the reaction mixture was diluted with saturated aqueous sodium carbonate solutions (20 mL) and ethyl acetate (20 mL). The layers were split and the organic layer was washed with brine (20 mL), was dried over anhydrous sodium sulfate, and was concentrated under reduced pressure. The crude residue purified via SiO₂ column chromatography (40 g SiO₂ Combiflash HP Gold Column, 0-100% ethyl acetate/hexanes) to afford the Cbz protected intermediate, which was taken up into TFA (1 mL) and the resulting mixture was heated to 50° C. After 2 h, the crude residue was purified by preparatory HPLC (Phenominex Luna 5 u C18(2) 100 Å 100×30 mm column, 5-100% acetonitrile/water gradient) to afford the product as the TFA salt. ¹H NMR (400 MHz, Methanol-d4) * denotes major diastereomer δ 8.05 (s, 1H), 8.00 (s, 1H*), 7.40-7.12 (m, 5H, 5H*), 6.98 (d, J=4.7 Hz, 1H), 6.93 (d, J=4.8 Hz, 1H*), 5.55 (d, J=5.2 Hz, 1H), 5.53 (d, J=5.0 Hz, 1H*), 4.57-4.50 (m, 1H, 1H*), 4.50-4.33 (m, 3H, 3H*), 4.08-3.96 (m, 2H, 2H*), 3.92-3.84 (m, 1H, 1H*), 2.87 (t, J=7.8 Hz, 2H*), 2.83-2.75 (m, 2H), 1.87-1.73 (m, 2H, 2H*), 1.73-1.24 (m, 10H, 10H*), 0.94-0.81 (m, 6H, 6H*). ³¹P NMR (162 MHz, Methanol-d4) * denotes major diastereomer δ 3.76 (s) 3.49* (s). ¹⁹F NMR (376 MHz, Methanol-d4) δ −77.99. MS m/z=660.31 [M+1].

### Example 92. (2S)-neopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

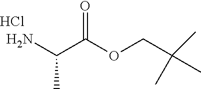

(S)-neopentyl 2-aminopropanoate hydrochloride. Boc-L-Alanine (3.8 g, 20 mmol) and neopentyl alcohol (2.1 g, 24 mmol) were dissolved in anhydrous dichloromethane (20 mL). The reaction mixture was cooled in an ice bath and stirred under atmospheric nitrogen. N-Ethyl-N′-(3-Dimethylaminopropyl)carbodiimide hydrochloride (5.8 g, 30 mmol) was added in 3 portions. 4-(Dimethylamino)pyridine (244 mg, 2 mmol) was added in one portion. Reaction was stirred for 30 min and then the ice bath was removed. Reaction was stirred at room temperature for 5 h. More neopentyl alcohol (210 mg, 2.4 mmol) and N-Ethyl-N′-(3-Dimethylaminopropyl)carbodiimide hydrochloride (500 mg) were added. Reaction was stirred for 16 h. The reaction mixture was diluted with dichloromethane (30 mL) and washed with 5% aqueous citric acid solution (15 mL), saturated aqueous sodium bicarbonate solution (15 mL) and brine (15 mL). Organic layer was dried over anhydrous sodium sulfate and concentrated under reduced pressure. The crude product was purified via SiO₂ column chromatography (40 g SiO₂ Combiflash HP Gold Column 0-20% ethyl acetate/hexanes). The resulting product was dissolved in 4 N hydrogen chloride in 1,4-dioxane (10 mL) and stirred for 30 min. Anhydrous ether (50 mL) was added and stirred for 30 min. Resulting solid was collected, washed with anhydrous ether (100 mL) and then dried under vacuum to give the intermediate. ¹H NMR (400 MHz, DMSO-d₆) δ 8.51 (s, 3H), 4.10 (q, J=7.2 Hz, 1H), 3.92 (d, J=10.4 Hz, 1H), 3.79 (d, J=10.4 Hz, 1H), 1.42 (d, J=7.2 Hz, 3H), 0.91 (s, 9H).

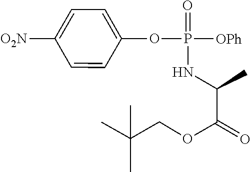 [SMILES: C[C@H](NP(=O)(Oc1ccccc1)Oc1ccc([N+](=O)[O-])cc1)C(=O)OCC(C)(C)C]

(2S)-neopentyl 2-(((4-nitrophenoxy)(phenoxy)phosphoryl)amino)propanoate. Phenyl dichlorophosphate (374 μL, 2.5 mmol) was dissolved in anhydrous dichloromethane (25 mL) and stirred under atmosphere nitrogen in an ice bath. (S)-neopentyl 2-aminopropanoate hydrochloride (489 mg, 2.5 mmol) was added in one portion. Triethylamine (768 μL, 5.5 mmol) was added dropwise and stirred for 1 h. More Triethylamine (384 μL, 2.75 mmol) was added dropwise and stirred for 20 mins. p-Nitrophenol (278 mg, 2 mmol) was added and ice bath was removed. Reaction was then stirred for 2 h. The reaction mixture was diluted with dichloromethane (20 mL) and washed with 5% aqueous sodium carbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, DMSO-d₆) δ 8.33-8.22 (m, 2H), 7.55-7.32 (m, 4H), 7.31-7.14 (m, 3H), 6.71 (m, 1H), 4.11-3.94 (m, 1H), 3.80-3.60 (m, 2H), 1.24 (m, 3H), 0.84 (d, J=1.6 Hz, 9H). ³¹P NMR (162 MHz, DMSO-d₆) δ −1.21, −1.40. MS m/z=436.9 [M+1], 435.1 [M−1].

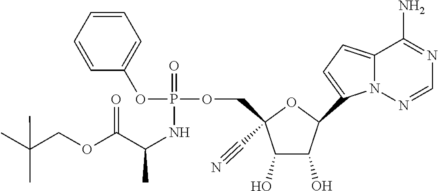 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC(C)(C)C]

(2S)-neopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. Intermediate 2 (50 mg, 0.116 mmol) and (2S)-neopentyl 2-(((4-nitrophenoxy)(phenoxy)phosphoryl)amino)propanoate (61 mg, 0.139 mmol) were dissolved in anhydrous tetrahydrofuran (3 mL). Magnesium chloride (17 mg, 0.174 mmol) was added in one portion. Reaction was warmed to 60° C. and stirred for 20 min. N,N-Diisopropylethylamine (50 μL, 0.29 mmol) was added, and the reaction was stirred for 20 h at 60° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (400 μL) was added dropwise and stirred at room temperature for 4 hrs. The reaction mixture was diluted with ethyl acetate (30 mL) and cooled in an ice bath. Saturated aqueous sodium bicarbonate solution was added dropwise to give pH of 10. Organic layer was collected and washed with brine (10 mL). Organic layer was dried over anhydrous sodium sulfate and concentrated under reduced pressure. The crude product was purified via SiO₂ column chromatography (4 g SiO₂ Combiflash HP Gold Column 0-5% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.38-7.08 (m, 5H), 6.84 (dd, J=4.5, 1.8 Hz, 1H), 6.73 (dd, J=4.6, 1.7 Hz, 1H), 5.49 (m, 1H), 4.66-4.57 (m, 1H), 4.54-4.29 (m, 3H), 3.94 (dq, J=9.8, 7.1 Hz, 1H), 3.86-3.60 (m, 2H), 1.30 (m, 3H), 0.89 (m, 9H). ³¹P NMR (162 MHz, Methanol-d₄) δ 3.28. MS m/z=589.0 [M+1], 586.9 [M−1].

Separation of the (S) and (R) Diastereomers. The product was purified via chiral preparatory HPLC (Chiralpak IA, 150×4.6 mm, Heptane 70% Ethanol 30%) to afford the diastereomers:

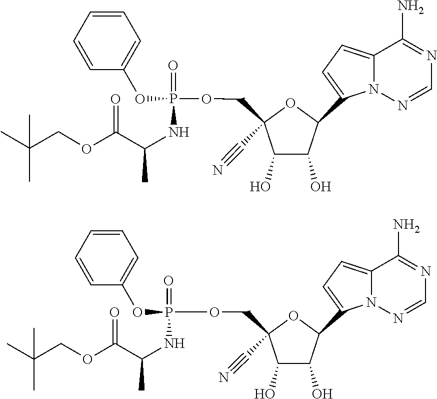 [SMILES: CC(O[P@](=O)(N[C@@H](C)C(=O)OCC(C)(C)C)Oc1ccccc1)[C@@]1(C#N)O[C@@H](c2ccc3c(N)ncnn23)[C@H](O)[C@@H]1O ; CC(O[P@@](=O)(N[C@@H](C)C(=O)OCC(C)(C)C)Oc1ccccc1)[C@@]1(C#N)O[C@@H](c2ccc3c(N)ncnn23)[C@H](O)[C@@H]1O]

### Example 93. First Eluting Diastereomer

¹H NMR (400 MHz, Methanol-d₄) δ 7.78 (s, 1H), 7.28 (dd, J=8.7, 7.1 Hz, 2H), 7.20-7.10 (m, 3H), 6.84 (d, J=4.5 Hz, 1H), 6.73 (d, J=4.5 Hz, 1H), 5.50 (d, J=5.0 Hz, 1H), 4.62 (t, J=5.3 Hz, 1H), 4.48 (m, 2H), 4.36 (dd, J=10.9, 5.2 Hz, 1H), 3.94 (dq, J=9.5, 7.2 Hz, 1H), 3.86-3.70 (m, 2H), 1.36-1.24 (m, 3H), 0.91 (m, 9H). ³¹P NMR (162 MHz, Methanol-d₄) δ 3.26.

### Example 94. Second Eluting Diastereomer

¹H NMR (400 MHz, Methanol-d4) δ 7.80 (s, 1H), 7.37-7.27 (m, 2H), 7.26-7.13 (m, 3H), 6.84 (d, J=4.5 Hz, 1H), 6.73 (d, J=4.5 Hz, 1H), 5.49 (d, J=5.0 Hz, 1H), 4.60 (t, J=5.3 Hz, 1H), 4.46 (d, J=5.6 Hz, 1H), 4.44-4.31 (m, 2H), 3.94 (dq, J=9.9, 7.1 Hz, 1H), 3.82-3.62 (m, 2H), 1.33-1.25 (m, 3H), 0.88 (m, 9H). ³¹P NMR (162 MHz, Methanol-d4) δ 3.27.

### Example 95. isopropyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutanecarboxylate

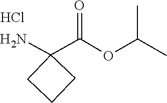

isopropyl 1-aminocyclobutanecarboxylate hydrochloride. 1-Aminocyclobutanecarboxylic acid hydrochloride (1.52 g, 10 mmol) was mixed with isopropyl alcohol (30 mL). Trimethylsilyl chloride (12.7 mL, 100 mmol) was added dropwise and stirred for 30 mins. Reaction mixture was heated to 80° C. and stirred for 20 h to give a clear solution. The reaction was concentrated under reduced pressure. Anhydrous ether (50 mL) was added and stirred for 30 min. Resulting solid was collected, washed with hexanes (50 mL) and dried under high vacuum to give the intermediate. ¹H NMR (400 MHz, DMSO-d₆) δ 8.73 (s, 3H), 5.00 (p, J=6.2 Hz, 1H), 2.46-2.32 (m, 4H), 2.02 (m, 2H), 1.26 (d, J=6.2 Hz, 6H).

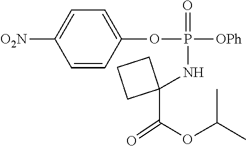 [SMILES: CC(C)OC(=O)C1(NP(=O)(Oc2ccccc2)Oc2ccc([N+](=O)[O-])cc2)CCC1]

isopropyl 1-(((4-nitrophenoxy)(phenoxy)phosphoryl)amino)cyclobutanecarboxylate. Phenyl dichlorophosphate (374 μL, 2.5 mmol) was dissolved in anhydrous dichloromethane (25 mL) and stirred under atmosphere nitrogen in an ice bath. isopropyl 1-aminocyclobutanecarboxylate hydrochloride (484 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 2 h.

Reaction was diluted with dichloromethane (20 mL) and washed with 1% aqueous citric acid solution (20 mL) followed with 5% aqueous sodium carbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, DMSO-d₆) δ 8.32-8.23 (m, 2H), 7.49-7.43 (m, 2H), 7.43-7.35 (m, 2H), 7.27-7.17 (m, 3H), 6.92 (d, J=11.7 Hz, 1H), 4.85 (p, J=6.2 Hz, 1H), 2.46-2.36 (m, 2H), 2.25-2.11 (m, 2H), 1.86-1.68 (m, 2H), 1.10 (d, J=6.2 Hz, 6H). ³¹P NMR (162 MHz, DMSO-d₆) δ −3.04. MS m/z=434.9 [M+1], 433.1 [M−1].

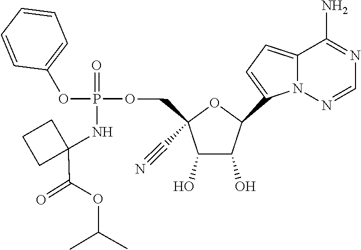 [SMILES: CC(C)OC(=O)C1(NP(=O)(OC[C@@]2(C#N)O[C@@H](c3ccc4c(N)ncnn34)[C@H](O)[C@@H]2O)Oc2ccccc2)CCC1]

isopropyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutanecarboxylate. Intermediate 2 (50 mg, 0.116 mmol) and isopropyl 1-(((4-nitrophenoxy)(phenoxy)phosphoryl)amino)cyclobutanecarboxylate (60 mg, 0.139 mmol) were dissolved in anhydrous tetrahydrofuran (3 mL). Magnesium chloride (22 mg, 0.232 mmol) was added in one portion. Reaction was warmed to 60° C. and stirred for 20 mins. N,N-Diisopropylethylamine (50 μL, 0.29 mmol) was added, and the reaction was stirred at 60° C. for 17 hrs. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (400 μL) was added dropwise and stirred for 4 hrs at room temperature.

Reaction was diluted with ethyl acetate (30 mL) and cooled in an ice bath. Saturated aqueous sodium bicarbonate solution was added dropwise to give pH of 10. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-5-10% methanol/dichloromethane). Fractions with the desired product were combined and concentrated under reduced pressure. Residue was dissolved in acetonitrile and water and freeze dried to give the product. ¹H NMR (400 MHz, Methanol-d₄) δ 7.78 (m, 1H), 7.36-7.09 (m, 5H), 6.83 (m, 1H), 6.72 (m, 1H), 5.53-5.45 (m, 1H), 4.98 (m, 1H), 4.63 (m, 1H), 4.55-4.31 (m, 3H), 2.56-2.34 (m, 2H), 2.34-2.13 (m, 2H), 1.94-1.75 (m, 2H), 1.26-1.17 (m, 6H). ³¹P NMR (162 MHz, Methanol-d₄) δ 2.03, 1.91. MS m/z=587.0 [M+1], 585.0 [M−1].

### Example 96. ((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-(benzyloxy)-3-(octadecyloxy)propyl) hydrogen phosphate

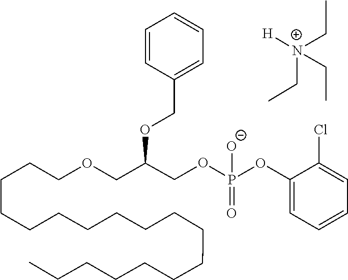

triethylammonium (R)-2-(benzyloxy)-3-(octadecyloxy)propyl (2-chlorophenyl) phosphate. 2-chlorophenyl phosphorodichloridate (0.371 mL, 2.3 mmol) was dissolved in ACN (9.2 mL). The resulting solution was cooled in an ice bath and 1,2,4-triazole (0.342 g, 4.95 mmol) was added, followed by Et₃N (0.689 mL, 4.95 mmol). The cold bath was removed and after 45 min a solution of (S)-2-(benzyloxy)-3-(octadecyloxy)propan-1-ol (1 g, 2.3 mmol) in pyridine (9.2 mL) was added. After stirring for 1 h 25 min at room temperature Et₃N (0.810 mL) and water (0.23 mL) were added to the reaction. The reaction was stirred for 10 min. A saturated aqueous NaHCO₃ solution was added and the mixture was stirred for an additional 10 min. The mixture was extracted with DCM (3×) and the combined organics were dried over Na₂SO₄, which was removed by filtration. The filtrate was concentrated and placed under high vacuum to yield the intermediate. This material was used directly as is in subsequent reactions. ¹H NMR (400 MHz, ACN-d₃) δ 7.61 (dt, J=8.2, 1.3 Hz, 1H), 7.40-7.24 (m, 6H), 7.17 (ddd, J=8.5, 7.4, 1.7 Hz, 1H), 6.99 (td, J=7.7, 1.5 Hz, 1H), 4.6 (m, 2H), 4.04-3.91 (m, 2H), 3.70 (pent, J=5.1 Hz, 1H), 3.51-3.42 (m, 2H), 3.36 (t, J=6.5 Hz, 2H), 2.96 (m, 6H), 1.49 (m, 2H), 1.27 (m, 30H), 1.19 (t, J=7.3 Hz, 9H), 0.88 (t, J=4 Hz, 3H). ³¹P NMR (162 MHz, ACN-d₃) δ −6.007 (s). MS m/z=624.97 [M+1].

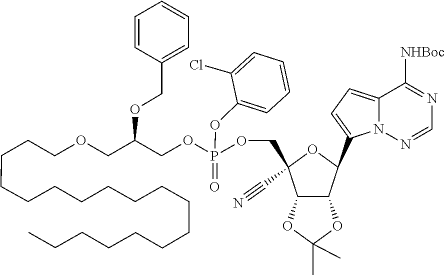 [SMILES: CCCCCCCCCCCCCCCCCCOC[C@H](COP(=O)(OC[C@@]1(C#N)O[C@@H](c2ccc3c(NC(=O)OC(C)(C)C)ncnn23)[C@@H]2OC(C)(C)O[C@@H]21)Oc1ccccc1Cl)OCc1ccccc1]

tert-butyl (7-((3aS,4S,6R,6aS)-6-(((((R)-2-(benzyloxy)-3-(octadecyloxy)propoxy)(2-chlorophenoxy)phosphoryl)oxy)methyl)-6-cyano-2,2-dimethyltetrahydrofuro[3,4-d][1,3]dioxol-4-yl)pyrrolo[2,1-f][1,2,4]triazin-4-yl)carbamate. Intermediate triethylammonium (R)-2-(benzyloxy)-3-(octadecyloxy)propyl (2-chlorophenyl) phosphate (0.337 g, 0.464 mmol) was dissolved in pyridine (4 mL). To this solution was added MSNT (0.275 g, 0.927 mmol), followed by Intermediate 2 (0.1 g, 0.232 mmol). To this solution was added N-methylimidazole (NMI) (0.046 mL, 0.579 mmol) and the reaction was stirred at room temperature, monitoring by for the disappearance of Intermediate 2 by LC/MS. The reaction was cooled in an ice bath and quenched by the slow addition of a saturated aqueous NaHCO₃ solution. The aqueous layer was diluted with a 1:1:1: mixture of water, a saturated aqueous NaHCO₃ solution and brine. The aqueous layer was extracted with DCM (3×) and the combined organics were dried over Na₂SO₄, which was removed by filtration. The filtrate was concentrated and the intermediate was isolated by silica gel column chromatography (25 g load cartridge, 40 g Combiflash HP Gold Column, eluent ramp from 100% hexanes to 60% EtOAc/hexanes). ¹H NMR (400 MHz, ACN-d₃) δ 8.44 (s, 1H), 8.12 (s, 1H), 7.47-7.39 (m, 1H), 7.36-7.32 (m, 1H), 7.31-7.21 (m, 5H), 7.20-7.06 (m, 3H), 6.91 (s, 1H), 5.69 (s, 1H), 5.25 (ddd, J=12.9, 6.6, 3.2 Hz, 1H), 5.04 (t, J=6.8 Hz, 1H), 4.60-4.13 (m, 6H), 3.72 (p, J=5.0 Hz, 1H), 3.43 (dd, J=7.8, 5.3 Hz, 2H), 3.35 (m, 2H), 1.71-1.67 (m, 3H), 1.59-1.50 (m, 9H), 1.47 (m, 2H), 1.35 (s, 3H), 1.32-1.20 (m, 30H), 0.87 (t, J=4 Hz, 3H). ³¹P NMR (162 MHz, ACN-d₃) δ −7.31 (s), −7.41 (s). MS m/z=1038.33 [M+1].

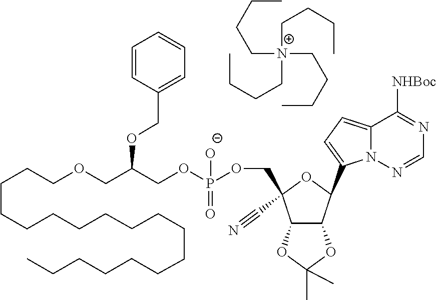 [SMILES: CCCCCCCCCCCCCCCCCCOC[C@H](COP(=O)([O-])OC[C@@]1(C#N)O[C@@H](c2ccc3c(NC(=O)OC(C)(C)C)ncnn23)[C@@H]2OC(C)(C)O[C@@H]21)OCc1ccccc1 ; CCCC[N+](CCCC)(CCCC)CCCC]

tetrabutylammonium (R)-2-(benzyloxy)-3-(octadecyloxy)propyl (((3aS,4R,6S,6aS)-6-(4-((tert-butoxycarbonyl)amino)pyrrolo[2,1-f][1,2,4]triazin-7-yl)-4-cyano-2,2-dimethyltetrahydrofuro[3,4-d][1,3]dioxol-4-yl)methyl) phosphate. Intermediate tert-butyl (7-((3aS,4S,6R,6aS)-6-(((((R)-2-(benzyloxy)-3-(octadecyloxy)propoxy)(2-chlorophenoxy)phosphoryl)oxy)methyl)-6-cyano-2,2-dimethyltetrahydrofuro [3,4-d][1,3]dioxol-4-yl)pyrrolo[2,1-f][1,2,4]triazin-4-yl)carbamate (0.532 g, 0.512 mmol) was dissolved in THF (5.7 mL) and pyridine (0.7 mL) and water (0.7 mL) were added. To the resulting clear homogenous solution was added a 1 M solution of TBAF in THF (1.537 mL, 1.537 mmol). The reaction was stirred at room temperature and reaction progress was monitored LC/MS. Upon completion the reaction was cooled in an ice bath and quenched with 2 pipettes of saturated aqueous NaHCO₃ solution. This solution was further diluted with 2 pipettes of water and the resulting mixture was extracted with DCM (4×). The aqueous phase was made acidic (pH-3) with 2 N HCl and then extracted with additional DCM (3×). The combined organic phases were extracted with brine (the pH of which was adjusted to 8 with a saturated aqueous NaHCO₃ solution) and dried over Na₂SO₄, which was removed by filtration. The filtrate was concentrated and the intermediate was isolated by silica gel column chromatography (12 g load cartridge, 80 g Combiflash HP Gold Column, eluent ramp from 100% DCM to 20% MeOH/DCM). ¹H NMR (400 MHz, CDCl₃) δ 8.18 (s, 1H), 8.03 (s, 1H), 7.36-7.09 (m, 6H), 6.86 (s, 1H), 5.72 (s, 1H), 5.12 (d, J=6.2 Hz, 1H), 5.02 (dd, J=6.6, 3.9 Hz, 1H), 4.64 (dd, J=18, 12 Hz, 2H), 4.13 (d, J=4.8 Hz, 2H), 3.94 (m, 2H), 3.77 (s, 1H), 3.58 (dd, J=10.4, 2.4 Hz, 1H), 3.48 (dd, J=10.6, 6.5 Hz, 1H), 3.35 (t, J=6 Hz, 2H), 3.32-3.22 (m, N⁺(CH₂CH₂CH₂CH₃)₄, 8H), 1.70 (s, 3H), 1.66-1.56 (m, N⁺(CH₂CH₂CH₂CH₃)₄, 8H), 1.54 (s, 9H), 1.52-1.47 (m, 2H), 1.36-1.45 (m, N⁺(CH₂CH₂CH₂CH₃)₄, 8H), 1.32 (s, 3H), 1.30-1.18 (m, 30H), 1.00-0.93 (m, N⁺(CH₂CH₂CH₂CH₃)₄, 12H), 0.86 (t, J=6.8 Hz, 3H). ³¹P NMR (162 MHz, DMSO-d₆) δ −0.875 (s). MS m/z=928.18 [M+1].

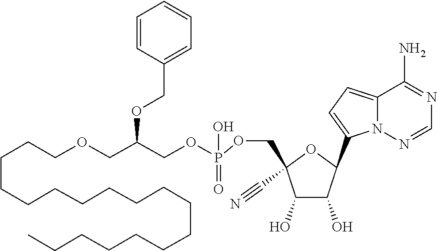 [SMILES: CCCCCCCCCCCCCCCCCCOC[C@H](COP(=O)(O)OC[C@@]1(C#N)O[C@@H](c2ccc3c(N)ncnn23)[C@H](O)[C@@H]1O)OCc1ccccc1]

((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-(benzyloxy)-3-(octadecyloxy)propyl) hydrogen phosphate. Intermediate tetrabutylammonium (R)-2-(benzyloxy)-3-(octadecyloxy)propyl (((3aS,4R,6S,6aS)-6-(4-((tert-butoxycarbonyl)amino)pyrrolo[2,1-f][1,2,4]triazin-7-yl)-4-cyano-2,2-dimethyltetrahydrofuro[3,4-d][1,3]dioxol-4-yl)methyl) phosphate (0.518 g, 0.558 mmol) was dissolved in THF (15 mL). To this solution was added a 12 M aqueous solution of HCl (3.837 mL, 46 mmol) at room temperature. The reaction was stirred at room temperature and reaction progress was monitored LC/MS. Upon completion the reaction was concentrated and the residue was coevaporated with THF (2×) and DCM (2×). The resulting residue was taken up in DCM and the product was isolated by silica gel column chromatography (12 g load cartridge, 40 g Combiflash HP Gold Column, eluent ramp from 100% DCM to 20% MeOH/DCM). ¹H NMR (400 MHz, Methanol-d₄) δ 7.79 (s, 1H), 7.33-7.17 (m, 5H), 6.83 (dd, J=11.6, 4.4 Hz, 2H), 5.55 (d, J=5.2 Hz, 1H), 4.65-4.49 (m, 4H), 4.20-4.08 (m, 2H), 3.95 (t, J=5.4 Hz, 2H), 3.71 (pent., J=5 Hz, 1H), 3.52 (dd, J=10.6, 3.8 Hz, 1H), 3.44 (dd, J=10.7, 6.0 Hz, 1H), 3.37 (td, J=6.6, 2.9 Hz, 2H), 1.50 (pent., J=6.7 Hz, 2H), 1.36-1.21 (m, 30H), 0.90 (t, J=6.7 Hz, 3H). ³¹P NMR (162 MHz, Methanol-d₄) δ −0.410. MS m/z=788.16 [M+1].

### Example 97. (2S)-ethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

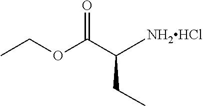

(S)-ethyl 2-aminobutanoate hydrochloride. To a mixture of (S)-ethyl 2-aminobutanoic acid (5 g, 48.5 mmol) and ethanol (28 mL) was added TMSCl (17 mL). The resulting mixture was stirred at 70° C. for 15 h and concentrated in vacuo. The resulting solid was tritulated with 5% EtOAc in hexanes, filtered, and washed with 5% EtOAc in hexanes several times, and dried under high vacuum for 15 h to afford the intermediate. ¹H NMR (400 MHz, Chloroform-d) δ 8.83 (s, 3H), 4.27 (p, J=7.0 Hz, 2H), 4.06 (q, J=5.6 Hz, 1H), 2.28-2.01 (m, 2H), 1.31 (t, J=7.0 Hz, 3H), 1.12 (t, J=7.4 Hz, 3H).

(2S)-ethyl 2-(((4-nitrophenoxy)(phenoxy)phosphoryl)amino)butanoate. Intermediate (S)-ethyl 2-aminobutanoate hydrochloride (1.20 g, 7.16 mmol) was converted to the intermediate by the same procedure used for Example 88. ¹H NMR (400 MHz, Chloroform-d) δ 8.34-8.09 (m, 2H), 7.45-7.30 (m, 4H), 7.27-7.14 (m, 3H), 4.15 (m, 2H), 4.09-3.97 (m, 1H), 3.90-3.74 (m, 1H), 1.88-1.62 (m, 2H), 1.24 (m, 3H), 0.86 (m, 3H). ³¹P NMR (162 MHz, Chloroform-d) δ −2.68, −2.73. MS m/z 409 (M+H)⁺.

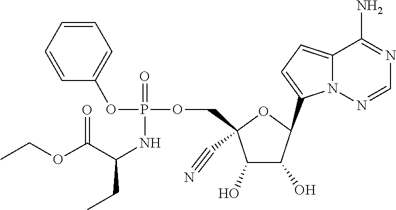 [SMILES: CCOC(=O)[C@H](CC)NP(=O)(OC[C@@]1(C#N)O[C@@H](c2ccc3c(N)ncnn23)[C@H](O)[C@@H]1O)Oc1ccccc1]

(2S)-ethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. The product was obtained from Intermediate 4 (50 mg, 0.12 mmol) and intermediate (2S)-ethyl 2-(((4-nitrophenoxy)(phenoxy)phosphoryl)amino)butanoate (71 mg, 0.18 mmol) by the same procedure used for Example 88. ¹H NMR (400 MHz, Methanol-d4) δ 7.79 (s, 0.56H), 7.78 (s, 0.44H), 7.39-7.06 (m, 5H), 6.93-6.80 (m, 1H), 6.73 (m, 1H), 5.49 (m, 1H), 4.63 (m, 1H), 4.56-4.26 (m, 3H), 4.19-3.94 (m, 2H), 3.84-3.65 (m, 1H), 1.69 (m, 1H), 1.58 (m, 1H), 1.18 (m, 3H), 0.82 (m 3H). ³¹P NMR (162 MHz, Methanol-d4) δ 3.61, 3.60. MS m/z=561 (M+H)⁺.

### Example 98. cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-valinate

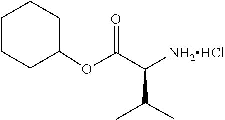

cyclohexyl L-valinate hydrochloride. 4 N hydrochloric acid (50 mL) was added to a solution of L-valine (5 g, 43 mmol) in cyclohexanol (45 g) and the resulting mixture was heated to 70° C. After 20 h, the reaction mixture was concentrated under reduced pressure at 70° C. The crude solid residue was taken up into hexanes (250 mL) and was stirred for 24 h. The resulting white crystalline solid was collected by vacuum filtration to afford the intermediate. ¹H NMR (400 MHz, methanol-d₄) δ 4.99-4.88 (m, 1H), 3.89 (d, J=4.5 Hz, 1H), 2.36-2.21 (m, 1H), 1.97-1.86 (m, 2H), 1.83-1.70 (m, 1H), 1.66-1.28 (m, 7H), 1.12-1.02 (m, 6H).

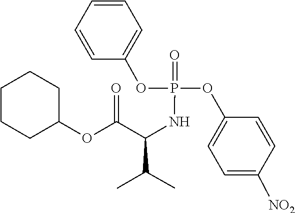 [SMILES: CC(C)[C@H](NP(=O)(Oc1ccccc1)Oc1ccc([N+](=O)[O-])cc1)C(=O)OC1CCCCC1]

cyclohexyl ((4-nitrophenoxy)(phenoxy)phosphoryl)-L-valinate. To a solution of intermediate cyclohexyl L-valinate hydrochloride (0.945 g, 4.74 mmol) and phenyl dichlorophosphate (0.705 mL, 4.74 mmol) in dichloromethane (23 mL) was added triethylamine (1.2 mL, 9.4 mmol) at 0° C. under and argon atmosphere. The resulting mixture was allowed to warm to RT and was stirred for 1 h. 4-Nitrophenol (660 mg, 4.74 mmol) and triethylamine (0.66 mL, 4.7 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, methanol-d₄) δ 8.31-8.24 (m, 2H), 7.49-7.18 (m, 7H), 4.72-4.62 (m, 1H), 3.74-3.66 (m, 1H), 2.09-1.95 (m, 1H), 1.79-1.64 (m, 2H), 1.57-1.47 (m, 1H), 1.44-1.25 (m, 7H), 0.92-0.84 (m, 6H). ³¹P NMR (162 MHz, methanol-d₄) δ −0.28 (s), −0.59 (s). MS m/z=476.85 [M+1].

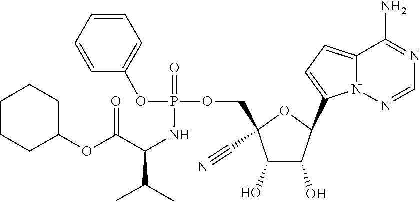 [SMILES: CC(C)[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-valinate. To a mixture of Intermediate 4 (20.0 mg, 0.060 mmol), intermediate cyclohexyl ((4-nitrophenoxy)(phenoxy)phosphoryl)-L-valinate (28.8 mg, 0.060 mmol), and magnesium chloride (5.7 mg, 0.060 mmol) was added acetonitrile (0.50 mL) at RT. The resulting suspension was warmed to 50° C., and was allowed to stir for 5 min. N,N-Diisopropylethylamine (0.026 mL, 0.151 mmol) was then added and the resulting mixture was stirred at 50° C. for 1 h. The reaction mixture was then allowed to cool to RT, and concentrated aqueous hydrochloric acid solution (12 M, 0.025 mL) was added. After 1 h, the reaction mixture was diluted with saturated aqueous sodium carbonate solution (20 mL) and ethyl acetate (20 mL). The layers were split and the organic layer was washed with brine (20 mL), was dried over anhydrous sodium sulfate, and was concentrated under reduced pressure. The crude residue was purified via SiO₂ column chromatography (12 g SiO₂ Combiflash HP Gold Column, 0-10% methanol/dichloromethane) to afford the product. ¹H NMR (400 MHz, methanol-d₄) δ 7.79 (s, 0.55H), 7.78 (s, 0.45H), 7.34-7.10 (m, 5H), 6.87-6.82 (m, 1H), 6.75-6.71 (m, 1H), 5.52-5.47 (m, 1H), 4.76-4.58 (m, 2H), 4.51-4.41 (m, 2H), 4.38-4.30 (m, 1H), 3.67-3.59 (m, 1H), 2.05-1.91 (m, 1H), 1.83-1.61 (m, 1H), 1.56-1.26 (m, 9H), 0.90-0.80 (m, 6H). ³¹P NMR (162 MHz, methanol-d₄) δ 4.15 (s), 4.09 (s). LCMS: MS m/z=629.32 [M+1], t_{R}=1.68 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.12 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=5.92 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 99. (2S)-ethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

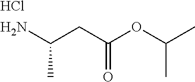

(S)-isopropyl 3-aminobutanoate hydrochloride. (S)-3-aminobutyric acid (1 g, 10 mmol) was mixed with isopropyl alcohol (30 mL)l. Trimethylsilyl chloride (12.7 mL, 100 mmol) was added dropwise and stirred for 30 mins. Reaction mixture was heated to 80° C. and stirred for 20 hrs. More Trimethylsilyl chloride (6 mL) was added dropwise. Reaction was stirred at 80° C. for 20 hrs to give a clear solution. Reaction was concentrated under reduced pressure to give a paste which was mixed with anhydrous ether (100 mL) and stirred for 5 mins. Solid was collected, washed with anhydrous ether (50 mL) and dried under high vacuum to give the intermediate. ¹H NMR (400 MHz, DMSO-d₆) δ 8.20 (s, 3H), 4.91 (p, J=6.3 Hz, 1H), 3.49-3.38 (m, 1H), 2.73 (dd, J=16.3, 5.5 Hz, 1H), 2.57-2.49 (m, 1H), 1.19 (m, 9H).

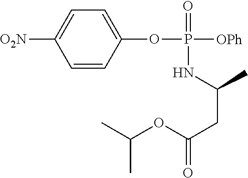 [SMILES: CC(C)OC(=O)C[C@H](C)NP(=O)(Oc1ccccc1)Oc1ccc([N+](=O)[O-])cc1]

(3S)-isopropyl 3-(((4-nitrophenoxy)(phenoxy)phosphoryl)amino)butanoate. Phenyl dichlorophosphate (374 μL, 2.5 mmol) was dissolved in anhydrous dichloromethane (25 mL) and stirred under atmosphere nitrogen in an ice bath. (S)-isopropyl 3-aminobutanoate hydrochloride (454 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 40 mins. p-Nitrophenol (278 mg, 2 mmol) was added and ice bath was removed. Reaction was then stirred for 2 hrs. More p-nitrophenol (50 mg) was added and stirred for 1 hr. Reaction was diluted with dichloromethane (20 mL) and washed with 1% aqueous citric acid solution (20 mL) followed with 5% aqueous sodium carbonate solution (20 mL). Organic layer was dried over anhydrous sodium sulfate and concentrated under reduced pressure. The crude product was purified via SiO₂ column chromatography (24 g SiO₂ Combiflash HP Gold Column 0-40% ethyl acetate/hexanes) to afford the intermediate. ¹H NMR (400 MHz, DMSO-d₆) δ 8.33-8.23 (m, 2H), 7.52-7.34 (m, 4H), 7.27-7.16 (m, 3H), 6.18 (ddd, J=13.4, 9.8, 1.8 Hz, 1H), 4.79 (pd, J=6.3, 4.4 Hz, 1H), 3.78-3.57 (m, 1H), 2.43-2.22 (m, 2H), 1.10 (m, 6H), 1.05 (m, 3H). ³¹P NMR (162 MHz, DMSO-d₆) δ −0.90, −0.98. MS m/z=422.9 [M+1], 421.1 [M−1].

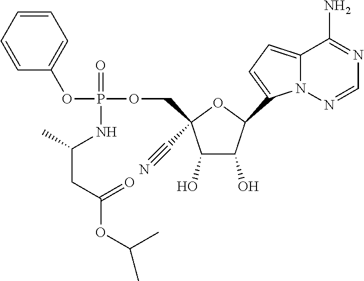 [SMILES: CC(C)OC(=O)C[C@H](C)NP(=O)(OC[C@@]1(C#N)O[C@@H](c2ccc3c(N)ncnn23)[C@H](O)[C@@H]1O)Oc1ccccc1]

(3S)-isopropyl 3-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. Intermediate 4 (50 mg, 0.15 mmol) and (3S)-isopropyl 3-(((4-nitrophenoxy)(phenoxy)phosphoryl)amino)butanoate (76 mg, 0.18 mmol) were dissolved in anhydrous tetrahydrofuran (3 mL). Magnesium chloride (29 mg, 0.3 mmol) was added in one portion. Reaction was warmed to 50° C. and stirred for 20 mins. N,N-Diisopropylethylamine (65 μL, 0.375 mmol) was added, and the reaction was stirred for 15 hrs at 50° C. Reaction was cooled to room temperature, diluted with ethyl acetate (30 mL) and washed with 5% aqueous sodium carbonate solution (3×20 mL) and then brine (20 mL), dried over anhydrous sodium sulfate, and concentrated under reduced pressure. Residue was dissolved in acetonitrile (2 mL) and stirred in an ice bath. 12 M hydrochloric acid (400 μL) was added dropwise and stirred at room temperature for 4 hrs. Reaction was diluted with ethyl acetate (30 mL) and cooled in an ice bath. Saturated aqueous sodium bicarbonate solution was added dropwise to give pH of 10. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-5-10% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.78 (M, 1H), 7.37-7.24 (m, 2H), 7.24-7.10 (m, 3H), 6.84 (m, 1H), 6.73 (m, 1H), 5.53-5.46 (m, 1H), 4.97-4.89 (m, 1H), 4.66-4.57 (m, 1H), 4.48 (t, J=5.4 Hz, 1H), 4.45-4.26 (m, 2H), 3.75-3.61 (m, 1H), 2.47-2.23 (m, 2H), 1.17 (m, 6H), 1.11 (m, 3H). ³¹P NMR (162 MHz, Methanol-d₄) δ 3.83, 3.74. MS m/z=575.0 [M+1], 573.0 [M−1].

### Example 100. 2-ethylbutyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutanecarboxylate

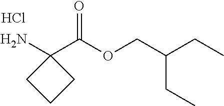

2-ethylbutyl 1-aminocyclobutanecarboxylate hydrochloride. 1-aminocyclobutanecarboxylic acid hydrochloride (1.52 g, 10 mmol) was mixed with anhydrous tetrahydrofuran (20 mL). 2-Ethyl-1-butanol (1.5 mL, 12 mmol) was added. Trimethylsilyl chloride (12.7 mL, 100 mmol) was added dropwise and stirred for 30 mins. Reaction mixture was heated to 80° C. and stirred for 20 hrs. More 2-Ethyl-1-butanol (3 mL) was added and stirred at 80° C. for 2 days. Reaction was concentrated under reduced pressure. 2-Ethyl-1-butanol (15 mL) was added. Trimethylsilyl chloride (10 mL) was added dropwise. Reaction mixture was heated to 80° C. and stirred for 20 hrs. Reaction was concentrated under reduced pressure to give the intermediate that was dried under high vacuum and used without further purification. ¹H NMR (400 MHz, DMSO-d₆) δ 8.85 (s, 3H), 4.12 (d, J=5.4 Hz, 2H), 2.55-2.36 (m, 4H), 2.11-1.93 (m, 2H), 1.63-1.45 (m, 1H), 1.35 (p, J=7.3 Hz, 4H), 0.87 (t, J=7.4 Hz, 6H).

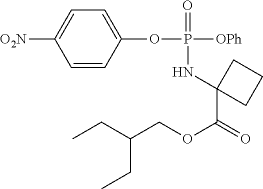 [SMILES: CCC(CC)COC(=O)C1(NP(=O)(Oc2ccccc2)Oc2ccc([N+](=O)[O-])cc2)CCC1]

2-ethylbutyl 1-(((4-nitrophenoxy)(phenoxy)phosphoryl)amino)cyclobutanecarboxylate. Phenyl dichlorophosphate (374 μL, 2.5 mmol) was dissolved in anhydrous dichloromethane (25 mL) and stirred under atmosphere nitrogen in an ice bath. 2-ethylbutyl 1-aminocyclobutane carboxylate hydrochloride (590 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 1 hr. More p-nitrophenol (55 mg) was added and stirred for 1 hr. Reaction was diluted with dichloromethane (20 mL) and washed with 5% aqueous sodium carbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, DMSO-d₆) δ 8.34-8.23 (m, 2H), 7.50-7.33 (m, 4H), 7.21 (m, 3H), 6.95 (m, 1H), 3.90 (d, J=5.6 Hz, 2H), 2.42 (m, 2H), 2.29-2.11 (m, 2H), 1.88-1.68 (m, 2H), 1.38 (m, 1H), 1.25 (m, 4H), 0.77 (m, 6H). ³¹P NMR (162 MHz, DMSO-d₆) δ −3.13. MS m/z=477.0 [M+1], 475.2 [M−1].

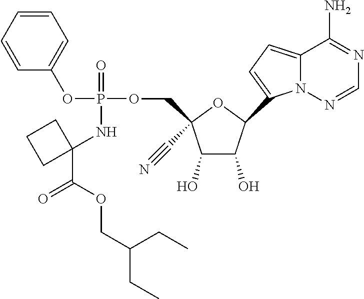 [SMILES: CCC(CC)COC(=O)C1(NP(=O)(OC[C@@]2(C#N)O[C@@H](c3ccc4c(N)ncnn34)[C@H](O)[C@@H]2O)Oc2ccccc2)CCC1]

2-ethylbutyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutanecarboxylate. Intermediate 4 (50 mg, 0.15 mmol) and 2-ethylbutyl 1-(((4-nitrophenoxy)(phenoxy)phosphoryl)amino)cyclobutanecarboxylate (76 mg, 0.18 mmol) were dissolved in anhydrous tetrahydrofuran (3 mL). Magnesium chloride (29 mg, 0.3 mmol) was added in one portion. Reaction was warmed to 50° C. and stirred for 20 mins. N,N-Diisopropylethylamine (65 μL, 0.375 mmol) was added, and the reaction was stirred for 15 hrs at 50° C. Reaction was cooled to room temperature, diluted with ethyl acetate (30 mL) and washed with 5% aqueous sodium carbonate solution (3×20 mL) and brine (20 mL), dried over anhydrous sodium sulfate and concentrated under reduced pressure. Residue was dissolved in acetonitrile (2 mL) and stirred in an ice bath. 12 M hydrochloric acid (300 μL) was added dropwise and stirred at room temperature for 80 min. Reaction was diluted with ethyl acetate (30 mL) and cooled in an ice bath. Saturated aqueous sodium bicarbonate solution was added dropwise to give pH of 10. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-5-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.78 (m, 1H), 7.37-7.09 (m, 5H), 6.83 (m, 1H), 6.72 (m, 1H), 5.51-5.49 (m, 1H), 4.62 (m, 1H), 4.53-4.32 (m, 3H), 4.07-3.99 (m, 2H), 2.57-2.36 (m, 2H), 2.36-2.18 (m, 2H), 1.96-1.76 (m, 2H), 1.58-1.43 (m, 1H), 1.43-1.29 (m, 4H), 0.94-0.80 (m, 6H). ³¹P NMR (162 MHz, Methanol-d₄) δ 1.95, 1.83. MS m/z=629.1 [M+1], 627.0 [M−1].

### Example 101. (2S)-pentan-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

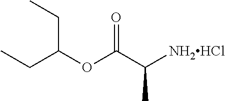

(S)-pentan-3-yl 2-aminopropanoate hydrochloride. To a mixture of L-alaninate (5 g, 56.12 mmol) and 3-hydroxypentane (50 mL) was added TMSCl (20 mL). The resulting mixture was stirred at 70° C. for 15 h and concentrated in rotary evaporator at 80° C. The resulting solid was triturated with 5% EtOAc in hexanes, filtered, and washed with 5% EtOAc in hexanes several times, and dried under high vacuum over night to give the intermediate. ¹H NMR (400 MHz, Chloroform-d) δ 8.79 (s, 3H), 4.83 (p, J=6.2 Hz, 1H), 4.19 (p, J=6.5 Hz, 1H), 1.72 (d, J=7.2 Hz, 3H), 1.67-1.52 (m, 4H), 0.88 (td, J=7.5, 1.7 Hz, 6H).

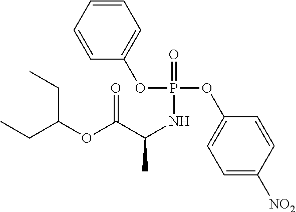 [SMILES: CCC(CC)OC(=O)[C@H](C)NP(=O)(Oc1ccccc1)Oc1ccc([N+](=O)[O-])cc1]

(2S)-pentan-3-yl 2-(((4-nitrophenoxy)(phenoxy)phosphoryl)amino)propanoate. Intermediate (S)-pentan-3-yl 2-aminopropanoate hydrochloride (1.00 g, 5.11 mmol) was suspended in methylene chloride (15 mL), cooled to −78° C., and phenyl dichlorophosphate (0.76 mL, 5.11 mmol) added quickly. Triethylamine (1.42 mL, 10.22 mmol) was added over 30 min at −78° C. and the resulting mixture was stirred at −78° C. for 30 min. Then 4-nitrophenol (711 mg, 5.11 mmol) was added in one portion and triethylamine (0.71 mL, 5.11 mmol) was added over 30 min at −78° C. The mixture was stirred for 30 min at −78° C., washed with water and brine, dried over sodium sulfate, and concentrated in vacuo. The residue was purified by silica gel column chromatography (EtOAc 0 to 20% in hexanes) to give the intermediate. ¹H NMR (400 MHz, Chloroform-d) δ 8.22 (m, 2H), 7.46-7.30 (m, 4H), 7.31-7.14 (m, 3H), 4.78 (m, 1H), 4.27-4.04 (m, 1H), 3.98-3.77 (m, 1H), 1.72-1.45 (m, 4H), 1.42 (m, 3H), 0.84 (m, 6H). ³¹P NMR (162 MHz, Chloroform-d) δ −2.99, −3.06. MS m/z=437 (M+H)⁺.

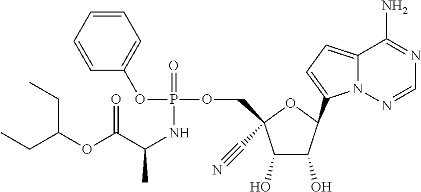 [SMILES: CCC(CC)OC(=O)[C@H](C)NP(=O)(OC[C@@]1(C#N)O[C@@H](c2ccc3c(N)ncnn23)[C@H](O)[C@@H]1O)Oc1ccccc1]

(2S)-pentan-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. To a mixture of Intermediate 4 (66 mg, 0.30 mmol), intermediate (2S)-pentan-3-yl 2-(((4-nitrophenoxy)(phenoxy)phosphoryl)amino)propanoate (170 mg, 0.39 mmol), and MgCl₂ (28 mg, 0.30 mmol) in DMF (3 mL) was added N,N-diisopropylethylamine (0.087 mL, 0.50 mmol) dropwise at room temperature. The resulting mixture was stirred at 60° C. for 15 h and purified by HPLC (ACN 0 to 100% in water) to give an intermediate which, was dissolved in ACN (3 mL) and C—HCl (0.1 mL) was added. The resulting mixture was stirred at 50° C. for 2 h, and purified by preparative HPLC (Phenominex Synergi 4 u Hydro-RR 80 Å 150×30 mm column, 5-100% acetonitrile/water gradient) to give the product. ¹H NMR (400 MHz, Methanol-d4) δ 7.79 (m, 1H), 7.36-7.07 (m, 5H), 6.84 (m, 1H), 6.73 (m, 1H), 5.50 (m, 1H), 4.76-4.59 (m, 2H), 4.54-4.40 (m, 2H), 4.34 (m, 1H), 3.89 (m, 1H), 1.63-1.42 (m, 4H), 1.27 (m, 3H), 0.91-0.75 (m, 6H). ³¹P NMR (162 MHz, Methanol-d4) δ 3.37, 3.29. MS m/z=589 (M+H)⁺.

### Example 102. (2R)-neopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

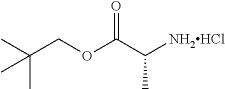

(R)-neopentyl 2-aminopropanoate hydrochloride. The intermediate was obtained from intermediate (R)-2-aminopropanoic acid (500 mg, 5.61 mmol) and intermediate neopentyl alcohol (5.0 g, 56.7 mmol) by using the same procedure used for Example 97.

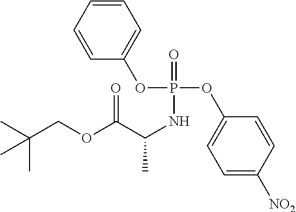 [SMILES: C[C@@H](NP(=O)(Oc1ccccc1)Oc1ccc([N+](=O)[O-])cc1)C(=O)OCC(C)(C)C]

(2R)-neopentyl 2-(((4-nitrophenoxy)(phenoxy)phosphoryl)amino)propanoate. Intermediate (R)-neopentyl 2-aminopropanoate hydrochloride (1.0 g, 5.11 mmol) was converted to the intermediate by the same procedure used for Example 88. ¹H NMR (400 MHz, Chloroform-d) δ 8.22 (m, 2H), 7.43-7.30 (m, 4H), 7.28-7.15 (m, 3H), 4.25-4.13 (m, 1H), 3.97-3.88 (m, 1H), 3.86 (dd, J=10.6, 1.2 Hz, 1H), 3.78 (dd, J=10.5, 3.4 Hz, 1H), 1.43 (m, 3H), 0.92 (m, 9H). ³¹P NMR (162 MHz, Chloroform-d) δ −3.01, −3.06. MS 437 (M+H)⁺.

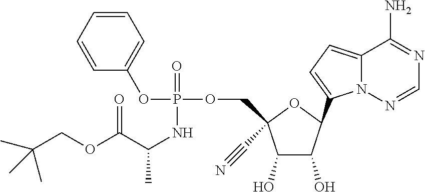 [SMILES: C[C@@H](NP(=O)(OC[C@@]1(C#N)O[C@@H](c2ccc3c(N)ncnn23)[C@H](O)[C@@H]1O)Oc1ccccc1)C(=O)OCC(C)(C)C]

(2R)-neopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. The product was obtained from Intermediate 4 (80 mg, 0.19 mmol) and intermediate (2R)-neopentyl 2-(((4-nitrophenoxy)(phenoxy)phosphoryl)amino)propanoate (198 mg, 0.45 mmol) by the same procedure used for Example 88. ¹H NMR (400 MHz, Methanol-d4) δ 7.80 (s, 0.5H), 7.78 (s, 0.5H), 7.37-7.25 (m, 2H), 7.23-7.08 (m, 3H), 6.84 (m, 1H), 6.73 (m, 1H), 5.50 (m, 1H), 4.62 (m, 1H), 4.52-4.41 (m, 2H), 4.35 (m, 1H), 3.96 (m, 1H), 3.83 (m, 1H), 3.70 (m, 1H), 1.31 (dd, J=7.2, 1.0 Hz, 1.5H), 1.24 (dd, J=7.2, 1.2 Hz, 1.5H), 0.89 (d, J=1.7 Hz, 9H). ³¹P NMR (162 MHz, Methanol-d4) δ 3.39, 3.08. MS m/z=589 (M+H)⁺.

### Example 103. (2S)-cyclohexyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

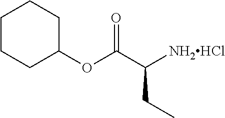

(S)-cyclohexyl 2-aminobutanoate hydrochloride. 4 N hydrochloric acid (12 mL) was added to a solution of (S)-2-aminobutanoic acid (1 g, 10 mmol) in cyclohexanol (5 mL) and the resulting mixture was heated to 70° C. After 20 h, the reaction mixture was concentrated under reduced pressure at 70° C. The crude solid residue was taken up into hexanes (150 mL) and was stirred for 4 h. The resulting white crystalline solid was collected by vacuum filtration to afford the intermediate. ¹H NMR (400 MHz, CD₃OD) δ 4.96-4.86 (m, 1H), 3.96 (t, J=6.1 Hz, 1H), 2.02-1.85 (m, 4H), 1.82-1.70 (m, 2H), 1.63-1.26 (m, 6H), 1.05 (t, J=7.5 Hz, 3H).

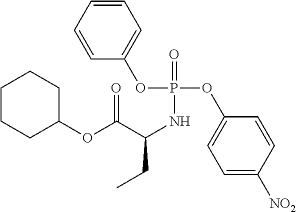 [SMILES: CC[C@H](NP(=O)(Oc1ccccc1)Oc1ccc([N+](=O)[O-])cc1)C(=O)OC1CCCCC1]

(2S)-cyclohexyl 2-(((4-nitrophenoxy)(phenoxy)phosphoryl)amino)butanoate. To a solution of (S)-cyclohexyl 2-aminobutanoate hydrochloride (0.878 g, 4.74 mmol) and phenyl dichlorophosphate (0.705 mL, 4.74 mmol) in dichloromethane (23 mL) was added triethylamine (1.2 mL, 9.4 mmol) at 0° C. under and argon atmosphere. The resulting mixture was allowed to warm to RT and was stirred for 1.5 h. 4-Nitrophenol (660 mg, 4.74 mmol) and triethylamine (0.66 mL, 4.7 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, DMSO-d₆) δ 8.28 (br d, J=9.1 Hz, 2H), 7.52-7.33 (m, 4H), 7.29-7.17 (m, 3H), 6.61 (td, J=12.8, 10.2 Hz, 1H), 4.65-4.55 (m, 1H), 3.74 (tdd, J=10.1, 7.8, 5.6 Hz, 1H), 1.75-1.37 (m, 6H), 1.36-1.13 (m, 5H), 0.76 (dd, J=7.4, 6.3 Hz, 3H). ³¹P NMR (162 MHz, DMSO-d₆) δ −0.79 (s), −1.03 (s). MS m/z=462.93 [M+1].

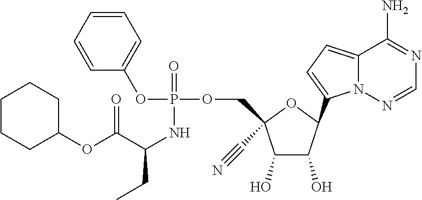 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC1CCCCC1]

(2S)-cyclohexyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. To a mixture of Intermediate 4 (35.0 mg, 0.106 mmol), intermediate (2S)-cyclohexyl 2-(((4-nitrophenoxy)(phenoxy)phosphoryl)amino)butanoate (49.0 mg, 0.106 mmol), and magnesium chloride (10.1 mg, 0.106 mmol) was added acetonitrile (0.50 mL) at RT. The resulting suspension was warmed to 50° C., and was allowed to stir for 5 min. N,N-Diisopropylethylamine (0.05 mL, 0.246 mmol) was then added and the resulting mixture was stirred at 50° C. for 1 h. The reaction mixture was then allowed to cool to RT, and concentrated aqueous hydrochloric acid solution (12 M, 0.123 mL, 1.5 mmol) was added. After 1 h, the reaction mixture was diluted with saturated aqueous sodium carbonate solutions (20 mL) and ethyl acetate (20 mL). The layers were split and the organic layer was washed with brine (20 mL), was dried over anhydrous sodium sulfate, and was concentrated under reduced pressure. The crude residue was purified by preparatory HPLC (Phenominex Synergi 4 u Hydro-RR 80 Å 150×30 mm column, 40-100% acetonitrile/water gradient) to afford the product. MS m/z=615.16 [M+H].

Separation of the (S) and (R) Diastereomers. The product was purified via chiral preparatory HPLC (Chiralpak IA, 150×4.6 mm, Heptane 80% Ethanol 20%) to afford the diastereomers:

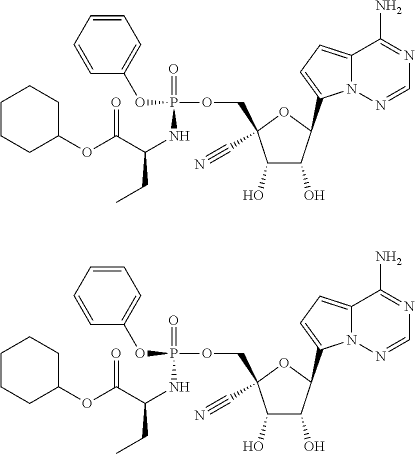 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OC1CCCCC1 ; CC[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]

### Example 104. First Eluting Diastereomer

¹H NMR (400 MHz, CD₃OD) δ 7.82 (s, 1H), 7.39-7.17 (m, 5H), 6.87 (d, J=4.5 Hz, 1H), 6.76 (d, J=4.5 Hz, 1H), 5.52 (d, J=5.0 Hz, 1H), 4.74-4.61 (m, 2H), 4.53-4.32 (m, 3H), 3.95 (p, J=6.2 Hz, 1H), 3.78 (dt, J=9.5, 6.6 Hz, 1H), 1.84-1.22 (m, 11H), 0.86 (t, J=7.4 Hz, 3H). ³¹P NMR (162 MHz, CD₃OD) δ 3.69 (s). MS m/z=615.16 [M+H]

### Example 105. Second Eluting Diastereomer

¹H NMR (400 MHz, CD₃OD) δ 7.81 (s, 1H), 7.37-7.27 (m, 2H), 7.24-7.14 (m, 3H), 6.88 (d, J=4.5 Hz, 1H), 6.76 (d, J=4.5 Hz, 1H), 5.54 (d, J=5.1 Hz, 1H), 4.78-4.69 (m, 1H), 4.65 (t, J=5.3 Hz, 1H), 4.55-4.47 (m, 2H), 4.39 (dd, J=10.9, 5.3 Hz, 1H), 3.95 (p, J=6.2 Hz, 1H), 3.77 (td, J=7.9, 5.4 Hz, 1H), 1.87-1.25 (m, 11H), 0.86 (t, J=7.4 Hz, 3H). ³¹P NMR (162 MHz, CD₃OD) δ 3.68 (br s). MS m/z=615.16 [M+H]

### Example 106. ethyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclopropanecarboxylate

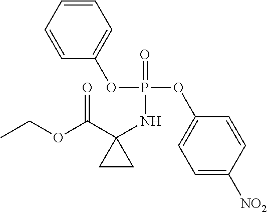 [SMILES: CCOC(=O)C1(NP(=O)(Oc2ccccc2)Oc2ccc([N+](=O)[O-])cc2)CC1]

ethyl 1-(((4-nitrophenoxy)(phenoxy)phosphoryl)amino)cyclopropanecarboxylate. Ethyl 1-aminocyclopropane-1-carboxylate hydrochloride (1.0 g, 6.04 mmol) was converted to the intermediate by the same procedure used for Example 88. ¹H NMR (400 MHz, Chloroform-d) δ 8.21 (m, 2H), 7.41-7.30 (m, 4H), 7.30-7.14 (m, 3H), 4.05 (m, 3H), 1.68-1.44 (m, 4H), 1.16 (t, J=7.1 Hz, 3H). ³¹P NMR (162 MHz, Chloroform-d) δ −3.16. MS m/z=407 (M+H)⁺.

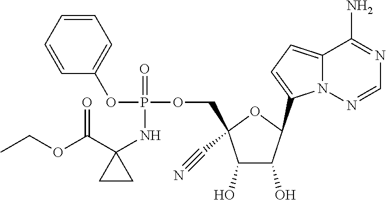 [SMILES: CCOC(=O)C1(NP(=O)(OC[C@@]2(C#N)O[C@@H](c3ccc4c(N)ncnn34)[C@H](O)[C@@H]2O)Oc2ccccc2)CC1]

ethyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclopropanecarboxylate. To a mixture of Intermediate 4 (100 mg, 0.30 mmol), intermediate ethyl 1-(((4-nitrophenoxy)(phenoxy)phosphoryl)amino)cyclopropanecarboxylate (147 mg, 0.36 mmol), and MgCl2 (43 mg, 0.45 mmol) in THF (4 mL) was added N,N-diisopropylethylamine (0.13 mL, 0.76 mmol) dropwise at room temperature. The resulting mixture was stirred at 60° C. for 15 h, diluted with EtOAc, washed with water and brine, dried with sodium sulfate, and concentrated in vacuo. The resulting residue was dissolved in ACN (3 mL) and c-HCl (0.5 mL) was added. The mixture was stirred for 2 h at room temperature and purified by preparative HPLC (Phenominex Synergi 4 u Hydro-RR 80 Å 150×30 mm column, 10-70% acetonitrile/water gradient) and then silica gel column chromatography (MeOH 0 to 15% in methylene chloride) to give the product. ¹H NMR (400 MHz, Methanol-d4) δ 7.80 (s, 0.5H), 7.77 (s, 0.5H), 7.38-7.23 (m, 2H), 7.24-7.11 (m, 3H), 6.84 (m, 1H), 6.73 (m, 1H), 5.53-5.45 (m, 1H), 4.64 (m, 1H), 4.54 (m, 0.5H), 4.52-4.40 (m, 2H), 4.37 (m, 0.5H), 4.13-4.01 (m, 2H), 1.32 (m, 2H), 1.17 (m, 3H), 1.07 (m, 2H). ³¹P NMR (162 MHz, Methanol-d4) δ 3.7, 3.61. MS m/z=559 (M+H)⁺.

### Example 107. ethyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclopropanecarboxylate

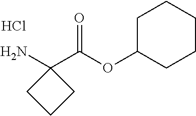 [SMILES: Cl ; NC1(C(=O)OC2CCCCC2)CCC1]

cyclohexyl 1-aminocyclobutanecarboxylate hydrochloride. 1-Aminocyclobutanecarboxylic acid hydrochloride (758 mg, 5 mmol) was mixed with cyclohexanol (5 mL). Trimethylsilyl chloride (6.4 mL, 50 mmol) was added dropwise and stirred for 30 mins at RT. Reaction mixture was heated to 80° C. and stirred for 20 hrs. More cyclohexanol (5 mL) and Trimethylsilyl chloride (6 mL) were added. Reaction was stirred at 80° C. and stirred for 20 hrs. Reaction was concentrated under reduced pressure. Residue was azeotroped with toluene (5×) to give gel/solid. Hexanes (100 mL) was added and stirred for 15 mins to give a solid which was collected, washed with hexanes (50 mL) and dried under high vacuum to give the intermediate. ¹H NMR (400 MHz, DMSO-d₆) δ 8.77 (s, 3H), 4.84 (tt, J=7.8, 3.6 Hz, 1H), 2.47-2.35 (m, 4H), 2.10-1.95 (m, 2H), 1.78 (m, 2H), 1.67 (m, 2H), 1.59-1.24 (m, 6H).

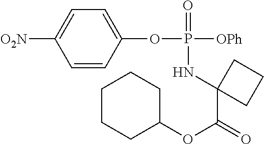 [SMILES: O=C(OC1CCCCC1)C1(NP(=O)(Oc2ccccc2)Oc2ccc([N+](=O)[O-])cc2)CCC1]

cyclohexyl 1-(((4-nitrophenoxy)(phenoxy)phosphoryl)amino)cyclobutanecarboxylate. Phenyl dichlorophosphate (374 μL, 2.5 mmol) was dissolved in anhydrous dichloromethane (25 mL) and stirred under atmosphere nitrogen in an ice bath. Cyclohexyl 1-aminocyclobutanecarboxylate hydrochloride (584 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 14 hrs. Reaction was diluted with dichloromethane (20 mL) and washed with 5% aqueous sodium carbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-30% ethyl acetate/hexanes) to afford the intermediate. ¹H NMR (400 MHz, DMSO-d₆) δ 8.34-8.23 (m, 2H), 7.50-7.43 (m, 2H), 7.43-7.35 (m, 2H), 7.27-7.16 (m, 3H), 6.90 (d, J=11.7 Hz, 1H), 4.64 (m, 1H), 2.43 (m, 2H), 2.28-2.14 (m, 2H), 1.88-1.72 (m, 2H), 1.72-1.52 (m, 4H), 1.42 (dd, J=11.7, 6.4 Hz, 1H), 1.36-1.16 (m, 5H). ³¹P NMR (162 MHz, DMSO-d₆) δ −3.06. MS m/z=474.9 [M+1], 473.2 [M−1].

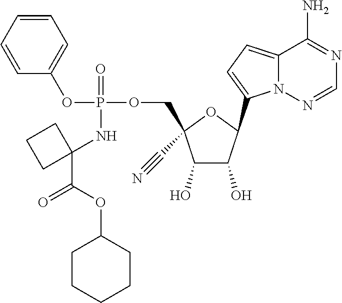 [SMILES: N#C[C@]1(COP(=O)(NC2(C(=O)OC3CCCCC3)CCC2)Oc2ccccc2)O[C@@H](c2ccc3c(N)ncnn23)[C@H](O)[C@@H]1O]

cyclohexyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutanecarboxylate. Intermediate 4 (50 mg, 0.15 mmol) and cyclohexyl 1-(((4-nitrophenoxy)(phenoxy)phosphoryl)amino)cyclobutanecarboxylate (85 mg, 0.18 mmol) were dissolved in anhydrous tetrahydrofuran (3 mL). Magnesium chloride (21 mg, 0.225 mmol) was added in one portion. Reaction was warmed to 50° C. and stirred for 20 mins. N,N-Diisopropylethylamine (65 μL, 0.375 mmol) was added, and the reaction was stirred for 14 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred at room temperature for 60 mins. Reaction was diluted with ethyl acetate (30 mL) and cooled in an ice bath. Saturated aqueous sodium bicarbonate solution was added dropwise to give pH of 10. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.78 (m, 1H), 7.37-7.09 (m, 5H), 6.87-6.81 (m, 1H), 6.72 (m, 1H), 5.52-5.47 (m, 1H), 4.75 (m, 1H), 4.67-4.59 (m, 1H), 4.54-4.32 (m, 3H), 2.47 (m, 2H), 2.34-2.17 (m, 2H), 1.97-1.59 (m, 7H), 1.58-1.24 (m, 5H). ³¹P NMR (162 MHz, Methanol-d₄) δ 2.00, 1.88. MS m/z=627.1 [M+1], 625.0 [M−1].

### Example 108. (2S)-cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

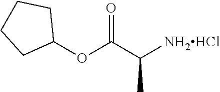

(S)-cyclopentyl 2-aminopropanoate hydrochloride. The intermediate was prepared from L-alaninate (3 g, 33.67 mmol) and cyclopentanol (31 mL) by the same procedure used for Intermediate 11. ¹H NMR (400 MHz, Chloroform-d) δ 8.73 (d, J=5.8 Hz, 3H), 5.26 (tt, J=5.7, 2.4 Hz, 1H), 4.14 (q, J=6.5 Hz, 1H), 1.99-1.41 (m, 11H).

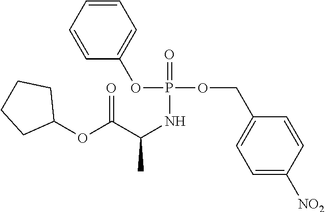 [SMILES: C[C@H](NP(=O)(OCc1ccc([N+](=O)[O-])cc1)Oc1ccccc1)C(=O)OC1CCCC1]

(2S)-cyclopentyl 2-(((4-nitrophenoxy)(phenoxy)phosphoryl)amino)propanoate. (S)-cyclopentyl 2-aminopropanoate hydrochloride (1.0 g, 5.16 mmol) was converted to the intermediate by the same procedure used for Intermediate 25. ¹H NMR (400 MHz, Chloroform-d) δ 8.22 (m, 2H), 7.45-7.29 (m, 4H), 7.29-7.11 (m, 3H), 5.17 (m, 1H), 4.18-3.97 (m, 1H), 3.93-3.75 (m, 1H), 1.92-1.79 (m, 2H), 1.76-1.51 (m, 6H), 1.38 (m, 3H). ³¹P NMR (162 MHz, Chloroform-d) δ −2.99, −3.03. MS m/z=435 (M+H)⁺.

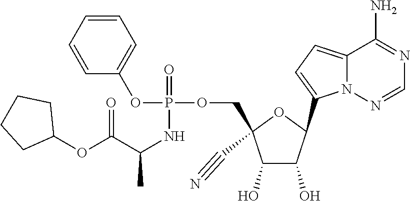 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCC1]

(2S)-cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. To a mixture of Intermediate 4 (79 mg, 0.24 mmol), intermediate (2S)-cyclopentyl 2-(((4-nitrophenoxy)(phenoxy)phosphoryl)amino)propanoate (155 mg, 0.326 mmol), and MgCl₂ (34 mg, 0.36 mmol) in THF (3 mL) was added N,N-diisopropylethylamine (0.104 mL, 0.60 mmol) dropwise at room temperature. The resulting mixture was stirred at 50° C. for 15 h, diluted with EtOAc, washed with water and brine, dried with sodium sulfate, and concentrated in vacuo. The resulting residue was dissolved in ACN (3 mL) and c-HCl (0.3 mL) was added. The mixture was stirred for 2 h and purified by preparative HPLC (Phenominex Synergi 4 u Hydro-RR 80 Å 150×30 mm column, 10-70% acetonitrile/water gradient) to give the product. ¹H NMR (400 MHz, Methanol-d4) δ 7.81 (m, 1H), 7.41-7.08 (m, 5H), 6.87 (m, 1H), 6.74 (m, 1H), 5.50 (m, 1H), 5.13-5.06 (m, 0.31H), 5.03 (td, J=6.0, 3.1 Hz, 0.69H), 4.69-4.55 (m, 1H), 4.55-4.21 (m, 3H), 3.84 (m, 1H), 1.93-1.43 (m, 8H), 1.24 (m, 3H). ³¹P NMR (162 MHz, Methanol-d4) δ 3.32, 2.75. MS m/z=587 (M+H)⁺.

### Example 109. (2S)-cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

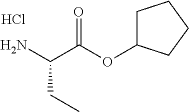

(S)-cyclopentyl 2-aminobutanoate hydrochloride. (S)-2-aminobutanoic acid (515 mg, 5 mmol) was mixed with cyclopentanol (10 mL). Thionyl chloride (1.1 mL, 15 mmol) was added dropwise. Reaction mixture was heated to 60° C. and stirred for 20 hrs. Reaction was concentrated under reduced pressure to give solid. Hexanes (100 mL) was added and stirred for 15 mins. Solid was collected, washed with hexanes (100 mL) and dried under high vacuum to give the intermediate. ¹H NMR (400 MHz, DMSO-d₆) δ 8.48 (s, 3H), 5.17 (ddt, J=5.9, 4.0, 2.1 Hz, 1H), 3.88 (t, J=6.0 Hz, 1H), 1.81 (m, 4H), 1.73-1.50 (m, 6H), 0.90 (t, J=7.5 Hz, 3H).

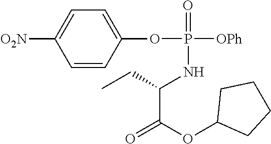 [SMILES: CC[C@H](NP(=O)(Oc1ccccc1)Oc1ccc([N+](=O)[O-])cc1)C(=O)OC1CCCC1]

(2S)-cyclopentyl 2-(((4-nitrophenoxy)(phenoxy)phosphoryl)amino)butanoate. Phenyl dichlorophosphate (374 μL, 2.5 mmol) was dissolved in anhydrous dichloromethane (25 mL) and stirred under atmosphere nitrogen in an ice bath. (S)-cyclopentyl 2-aminobutanoate hydrochloride (519 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 14 hrs. Reaction was diluted with dichloromethane (20 mL) and washed with water (20 mL), followed with 2% aqueous citric acid solution (20 mL). Organic layer was dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, DMSO-d₆) δ 8.34-8.22 (m, 2H), 7.53-7.31 (m, 4H), 7.30-7.15 (m, 3H), 6.60 (dt, J=13.4, 10.4 Hz, 1H), 4.98 (m, 1H), 3.72 (m, 1H), 1.82-1.67 (m, 2H), 1.67-1.40 (m, 8H), 0.75 (m, 3H). ³¹P NMR (162 MHz, DMSO-d₆) δ −0.82, −1.02. MS m/z=448.9 [M+1], 447.1 [M−1].

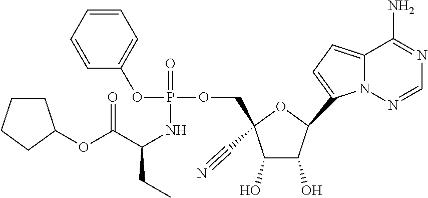 [SMILES: CC[C@H](NP(=O)(OC[C@@]1(C#N)O[C@@H](c2ccc3c(N)ncnn23)[C@H](O)[C@@H]1O)Oc1ccccc1)C(=O)OC1CCCC1]

(2S)-cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. Intermediate 4 (50 mg, 0.15 mmol) and (2S)-cyclopentyl 2-(((4-nitrophenoxy)(phenoxy)phosphoryl)amino)butanoate (81 mg, 0.18 mmol) were dissolved in anhydrous tetrahydrofuran (3 mL). Magnesium chloride (21 mg, 0.225 mmol) was added in one portion. Reaction was warmed to 50° C. and stirred for 30 mins. N,N-Diisopropylethylamine (65 μL, 0.375 mmol) was added, and the reaction was stirred for 14 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred at room temperature for 60 mins. Reaction was diluted with ethyl acetate (30 mL) and cooled in an ice bath. Saturated aqueous sodium bicarbonate solution was added dropwise to give pH of 9. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.36-7.10 (m, 5H), 6.84 (m, 1H), 6.73 (m, 1H), 5.53-5.47 (m, 1H), 5.19-4.93 (m, 1H), 4.62 (m, 1H), 4.53-4.39 (m, 2H), 4.35 (m, 1H), 3.72 (m, 1H), 1.90-1.43 (m, 10H), 0.82 (m, 3H). ³¹P NMR (162 MHz, Methanol-d₄) δ 3.66, 3.59. MS m/z=601.1 [M+1], 699.0 [M−1].

Separation of the (S) and (R) Diastereomers. The product was purified via chiral preparatory HPLC (Chiralpak IC, 150×4.6 mm, Heptane 80% Ethanol 20%) to afford the diastereomers:

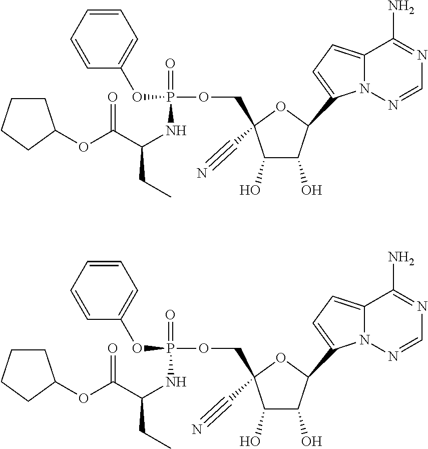 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OC1CCCC1 ; CC[C@H](N[P@](=O)(OC[C@@]1(C#N)O[C@@H](c2ccc3c(N)ncnn23)[C@H](O)[C@@H]1O)Oc1ccccc1)C(=O)OC1CCCC1]

### Example 110. First Eluting Diastereomer

¹H NMR (400 MHz, Methanol-d₄) δ 7.80 (s, 1H), 7.37-7.27 (m, 2H), 7.27-7.13 (m, 3H), 6.84 (d, J=4.5 Hz, 1H), 6.74 (d, J=4.5 Hz, 1H), 5.48 (d, J=4.9 Hz, 1H), 5.02 (tt, J=5.6, 2.4 Hz, 1H), 4.62 (t, J=5.3 Hz, 1H), 4.50-4.37 (m, 2H), 4.32 (dd, J=10.9, 5.3 Hz, 1H), 3.71 (ddd, J=9.6, 7.4, 5.7 Hz, 1H), 1.85-1.46 (m, 10H), 0.83 (t, J=7.4 Hz, 3H). ³¹P NMR (162 MHz, Methanol-d₄) δ 3.67.

### Example 111. Second Eluting Diastereomer

¹H NMR (400 MHz, Methanol-d₄) δ 7.78 (s, 1H), 7.33-7.23 (m, 2H), 7.20-7.10 (m, 3H), 6.85 (d, J=4.5 Hz, 1H), 6.73 (d, J=4.5 Hz, 1H), 5.50 (d, J=5.1 Hz, 1H), 5.09 (tt, J=5.6, 2.5 Hz, 1H), 4.62 (t, J=5.4 Hz, 1H), 4.54-4.41 (m, 2H), 4.36 (dd, J=10.9, 5.3 Hz, 1H), 3.77-3.65 (m, 1H), 1.91-1.48 (m, 10H), 0.82 (t, J=7.4 Hz, 3H). ³¹P NMR (162 MHz, Methanol-d₄) δ 3.61.

### Example 112. cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)acetate

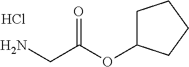

cyclopentyl 2-aminoacetate hydrochloride. Glycine (750 mg, 10 mmol) was mixed with cyclopentanol (10 mL). Thionyl chloride (2.6 mL) was added dropwise and stirred for 30 mins. Reaction mixture was heated to 60° C. and stirred for 20 hrs. Reaction was concentrated under reduced pressure and dried under high vacuum. Hexanes (100 mL) was added and stirred for 15 mins to give solid. Solid was collected, washed with hexanes (100 mL) and dried under high vacuum to give the intermediate. ¹H NMR (400 MHz, DMSO-d₆) δ 8.38 (s, 3H), 5.17 (tt, J=6.3, 2.5 Hz, 1H), 3.71 (s, 2H), 1.94-1.73 (m, 2H), 1.73-1.47 (m, 6H).

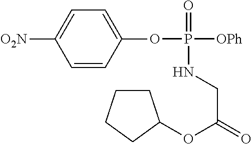 [SMILES: O=C(CNP(=O)(Oc1ccccc1)Oc1ccc([N+](=O)[O-])cc1)OC1CCCC1]

cyclopentyl 2-(((4-nitrophenoxy)(phenoxy)phosphoryl)amino)acetate. Phenyl dichlorophosphate (374 μL, 2.5 mmol) was dissolved in anhydrous dichloromethane (25 mL) and stirred under atmosphere nitrogen in an ice bath. Cyclopentyl 2-aminoacetate hydrochloride (449 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 3 hrs. Reaction was diluted with dichloromethane (20 mL) and washed with water (10 mL), followed with 2% aqueous citric acid solution (10 mL). Organic layer was dried over anhydrous sodium sulfate and concentrated under reduced pressure. The crude product was purified via SiO₂ column chromatography (24 g SiO₂ Combiflash HP Gold Column 0-30% ethyl acetate/hexanes) to afford the intermediate. ¹H NMR (400 MHz, DMSO-d₆) δ 8.34-8.21 (m, 2H), 7.53-7.43 (m, 2H), 7.43-7.34 (m, 2H), 7.29-7.15 (m, 3H), 6.53 (dt, J=14.3, 7.1 Hz, 1H), 5.04 (m, 1H), 3.71 (dd, J=14.9, 7.1 Hz, 2H), 1.77 (m, 2H), 1.54 (m, 6H). ³¹P NMR (162 MHz, DMSO-d₆) δ −0.11. MS m/z=420.9 [M+1], 419.1 [M−1].

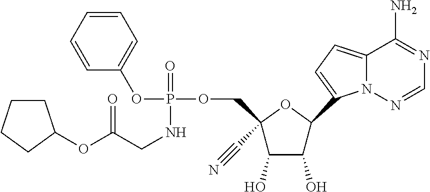 [SMILES: N#C[C@]1(COP(=O)(NCC(=O)OC2CCCC2)Oc2ccccc2)O[C@@H](c2ccc3c(N)ncnn23)[C@H](O)[C@@H]1O]

cyclopentyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)acetate. Intermediate 4 (50 mg, 0.15 mmol) and cyclopentyl 2-(((4-nitrophenoxy)(phenoxy)phosphoryl)amino)acetate (76 mg, 0.18 mmol) were dissolved in anhydrous tetrahydrofuran (3 mL). Magnesium chloride (21 mg, 0.225 mmol) was added in one portion. Reaction was warmed to 50° C. and stirred for 30 mins. N,N-Diisopropylethylamine (65 μL, 0.375 mmol) was added, and the reaction was stirred for 8 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred at room temperature for 60 mins. Reaction was diluted with ethyl acetate (30 mL) and cooled in an ice bath. Saturated aqueous sodium bicarbonate solution was added dropwise to give pH of 9. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.37-7.10 (m, 5H), 6.84 (m, 1H), 6.73 (m, 1H), 5.50 (m, 1H), 5.12 (m, 1H), 4.62 (m, 1H), 4.56-4.32 (m, 3H), 3.67-3.58 (m, 2H), 1.81 (m, 2H), 1.74-1.48 (m, 6H). ³¹P NMR (162 MHz, Methanol-d₄) δ 4.52, 4.36. MS m/z=573.1 [M+1], 571.0 [M−1].

Separation of the (S) and (R) Diastereomers. The product was purified via chiral preparatory HPLC (Chiralpak IC, 150×4.6 mm, Heptane 80% Ethanol 20%) to afford the diastereomers:

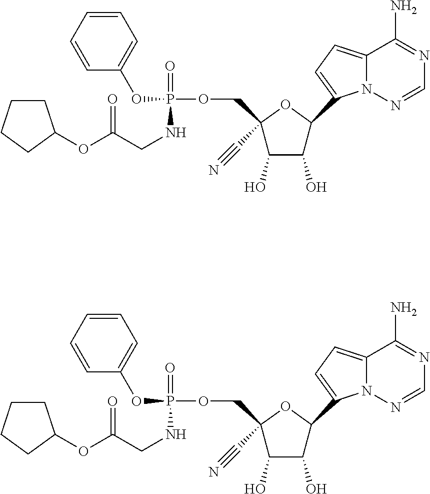 [SMILES: N#C[C@]1(CO[P@](=O)(NCC(=O)OC2CCCC2)Oc2ccccc2)O[C@@H](c2ccc3c(N)ncnn23)[C@H](O)[C@@H]1O ; N#C[C@]1(CO[P@@](=O)(NCC(=O)OC2CCCC2)Oc2ccccc2)O[C@@H](c2ccc3c(N)ncnn23)[C@H](O)[C@@H]1O]

### Example 113. First Eluting Diastereomer

¹H NMR (400 MHz, Methanol-d₄) δ 7.81 (s, 1H), 7.37-7.27 (m, 2H), 7.26-7.13 (m, 3H), 6.84 (d, J=4.5 Hz, 1H), 6.74 (d, J=4.5 Hz, 1H), 5.50 (d, J=5.2 Hz, 1H), 5.09 (tt, J=5.6, 2.5 Hz, 1H), 4.68-4.58 (m, 1H), 4.44 (m, 3H), 3.66-3.58 (m, 2H), 1.80 (m, 2H), 1.74-1.48 (m, 6H). ³¹P NMR (162 MHz, Methanol-d₄) δ 4.54.

### Example 114. Second Eluting Diastereomer

¹H NMR (400 MHz, Methanol-d₄) δ 7.79 (s, 1H), 7.34-7.25 (m, 2H), 7.23-7.12 (m, 3H), 6.87-6.82 (m, 1H), 6.73 (d, J=4.5 Hz, 1H), 5.50 (d, J=5.1 Hz, 1H), 5.15 (ddt, J=8.1, 5.4, 2.4 Hz, 1H), 4.65-4.57 (m, 1H), 4.51 (m, 2H), 4.36 (dd, J=10.8, 5.1 Hz, 1H), 3.67-3.55 (m, 2H), 1.83 (m, 2H), 1.75-1.50 (m, 6H). ³¹P NMR (162 MHz, Methanol-d₄) δ 4.39.

### Example 115. (2S)-pentan-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

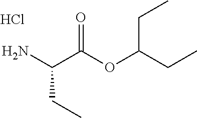

(S)-pentan-3-yl 2-aminobutanoate hydrochloride. L-2-aminobutyric acid (515 mg, 5 mmol) was mixed with 3-pentanol (10 mL). Trimethylsilyl chloride (6.4 mL, 50 mmol) was added dropwise and stirred for 30 mins. Reaction mixture was heated to 60° C. and stirred for 20 hrs. Reaction was concentrated under reduced pressure. Hexanes (100 mL) was added and stirred for 15 mins to give solid. Solid was collected, washed with hexanes (100 mL) and dried under high vacuum to give the intermediate. ¹H NMR (400 MHz, DMSO-d₆) δ 8.50 (s, 3H), 4.76 (tt, J=7.2, 5.1 Hz, 1H), 3.97 (t, J=5.9 Hz, 1H), 1.84 (m, 2H), 1.67-1.43 (m, 4H), 0.93 (t, J=7.5 Hz, 3H), 0.84 (td, J=7.4, 2.8 Hz, 6H).

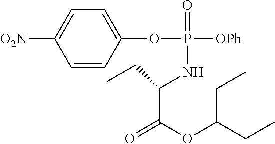 [SMILES: CCC(CC)OC(=O)[C@H](CC)NP(=O)(Oc1ccccc1)Oc1ccc([N+](=O)[O-])cc1]

(2S)-pentan-3-yl 2-(((4-nitrophenoxy)(phenoxy)phosphoryl)amino)butanoate. Phenyl dichlorophosphate (374 μL, 2.5 mmol) was dissolved in anhydrous dichloromethane (25 mL) and stirred under atmosphere nitrogen in an ice bath. (S)-pentan-3-yl 2-aminobutanoate hydrochloride (524 mg, 2.5 mmol) was added in one portion. Triethylamine (768 μL, 5.5 mmol) was added dropwise and stirred for 1 hr. More Triethylamine (384 μL, 2.75 mmol) was added dropwise and stirred for 20 mins. p-Nitrophenol (278 mg, 2 mmol) was added and ice bath was removed. Reaction was then stirred for 14 hrs. Reaction was diluted with dichloromethane (20 mL) and washed with water (10 mL), followed with 2% aqueous citric acid solution (10 mL). Organic layer was dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, DMSO-d₆) δ 8.31-8.23 (m, 2H), 7.52-7.33 (m, 4H), 7.28-7.15 (m, 3H), 6.62 (m, 1H), 4.62 (m, 1H), 3.85-3.71 (m, 1H), 1.74-1.31 (m, 6H), 0.84-0.67 (m, 9H). ³¹P NMR (162 MHz, DMSO-d₆) δ −0.77, −1.00. MS m/z=451.0 [M+1], 449.1 [M−1].

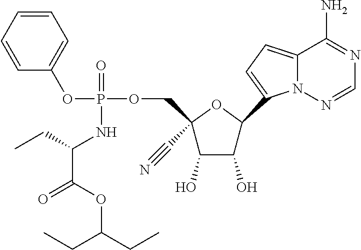 [SMILES: CCC(CC)OC(=O)[C@H](CC)NP(=O)(OC[C@@]1(C#N)O[C@@H](c2ccc3c(N)ncnn23)[C@H](O)[C@@H]1O)Oc1ccccc1]

(2S)-pentan-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. Intermediate 4 (50 mg, 0.15 mmol) and (2S)-pentan-3-yl 2-(((4-nitrophenoxy)(phenoxy)phosphoryl)amino)butanoate (81 mg, 0.18 mmol) were dissolved in anhydrous tetrahydrofuran (3 mL). Magnesium chloride (21 mg, 0.225 mmol) was added in one portion. Reaction was warmed to 50° C. and stirred for 20 mins. N,N-Diisopropylethylamine (65 μL, 0.375 mmol) was added, and the reaction was stirred for 16 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred in an ice bath for 60 mins. Reaction was diluted with ethyl acetate (30 mL) and cooled in an ice bath. Saturated aqueous sodium bicarbonate solution was added dropwise to give pH of 9. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.36-7.10 (m, 5H), 6.84 (m, 1H), 6.73 (m, 1H), 5.50 (m, 1H), 4.76-4.56 (m, 2H), 4.54-4.40 (m, 2H), 4.34 (m, 1H), 3.78 (m, 1H), 1.73 (m, 1H), 1.66-1.44 (m, 5H), 0.90-0.75 (m, 9H). ³¹P NMR (162 MHz, Methanol-d₄) δ 3.72, 3.63. MS m/z=603.1 [M+1], 601.0 [M−1].

### Example 116. (2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-2-cyclohexylacetate

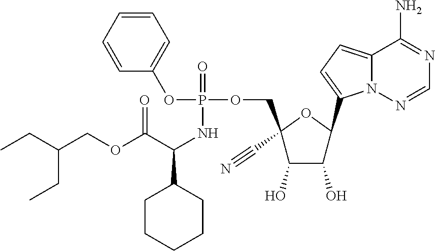 [SMILES: CCC(CC)COC(=O)[C@@H](NP(=O)(OC[C@@]1(C#N)O[C@@H](c2ccc3c(N)ncnn23)[C@H](O)[C@@H]1O)Oc1ccccc1)C1CCCCC1]

To a mixture of Intermediate 4 (40.0 mg, 0.093 mmol), Intermediate 35 (57.69 mg, 0.111 mmol), and magnesium chloride (13.24 mg, 0.139 mmol) was added THF (1.0 mL) at RT. The resulting suspension was warmed to 50° C., and was allowed to stir for 10 min. N,N-Diisopropylethylamine (0.040 mL, 0.232 mmol) was then added and the resulting mixture was stirred at 50° C. for 30 min. The reaction mixture was then allowed to cool to RT, and concentrated aqueous hydrochloric acid solution (12 M, 0.200 mL, 2.4 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.36 min, MS m/z=671.34 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.75 (br s, 2H), 7.39-7.20 (m, 3H), 7.23-7.04 (m, 2H), 6.84 (d, J=4.5, 1H), 6.70 (d, J=4.5, 1H), 6.09 (m, 1H), 5.96 (m, 1H), 5.47 (d, J=5.9 Hz, 1H), 5.37 (m, 1H), 4.45 (m, 1H), 4.39-4.22 (m, 2H), 3.99-3.81 (m, 2H), 3.77 (m, 1H), 3.62-3.44 (m, 1H), 1.67-0.65 (m, 22H). ³¹P NMR (162 MHz, DMSO-d₆) δ 4.06 (s), 3.90 (s).

### Example 117. (2R)-ethyl 1-((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)pyrrolidine-2-carboxylate

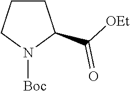 [SMILES: CCOC(=O)[C@@H]1CCCN1C(=O)OC(C)(C)C]

(S)-1-tert-butyl 2-ethyl pyrrolidine-1,2-dicarboxylate. (tert-butoxycarbonyl)-L-proline (10 g, 0.046 mol) was dissolved in CH₂Cl₂ (200 mL). To this solution was added EDCI (8.66 g, 0.056 mol), DMAP (1.70 g, 0.014 mol), and DIPEA (8.9 mL, 0.051 mol). EtOH (13.56 mL, 0.232 mL) was then added and the reaction was stirred at room temperature. After 2 hours the reaction was cooled in an ice bath and quenched via the pipette-wise addition of a saturated aqueous NaHCO₃ solution. The mixture was warmed to room temperature and further diluted with CH₂Cl₂ and a saturated aqueous NaHCO₃ solution. The layers were separated and the organic layer was washed with water. The organic phase was then washed with pH 3 water (water was added to the separatory funnel and the pH was adjusted by adding 0.5 N HCl), half saturated brine and dried over Na₂SO₄. The drying agent was removed by filtration. The filtrate was concentrated and the intermediate was isolated by silica gel column chromatography (25 g load cartridge, 220 g Combiflash HP Gold Column, eluent ramp from 100% hexanes to 35% EtOAc/hexanes). ¹H NMR (400 MHz, DMSO-d₆) δ 4.17-4.00 (m, 3H), 3.40-3.26 (m, 2H), 2.26-2.12 (m, 1H), 1.88-1.75 (m, 3H), 1.39 (s, Boc rotamer #1, 4H), 1.33 (s, Boc rotamer #2, 5H), 1.22-1.15 (m, 3H).

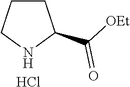

(S)-ethyl pyrrolidine-2-carboxylate hydrochloride. Intermediate (S)-1-tert-butyl 2-ethyl pyrrolidine-1,2-dicarboxylate (6.94 g, 0.029 mol) was dissolved in CH₂Cl₂ (20 mL). To this solution was added a 4 N solution of HCl in dioxane (35.66 mL, 0.143 mol). The resulting solution was stirred at room temperature and the progress of the reaction was monitored by TLC. After 1 hour 5 minutes the reaction was concentrated to an oil and then co-evaporated with CH₂Cl₂ twice. The resulting residue was placed under high vacuum for overnight and used as is in subsequent reactions. ¹H NMR (400 MHz, DMSO-d₆) δ 10.34 (brs, 1H), 9.03 (brs, 1H), 4.33 (t, J=9.1 Hz, 1H), 4.21 (quart, J=7.2 Hz, 2H), 3.28-3.13 (m, 2H), 2.31-2.20 (m, 1H), 2.04-1.84 (m, 3H), 1.24 (t, J=7.2 Hz, 3H).

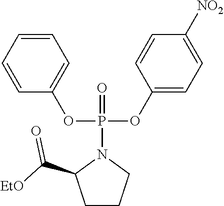 [SMILES: CCOC(=O)[C@@H]1CCCN1P(=O)(Oc1ccccc1)Oc1ccc([N+](=O)[O-])cc1]

(2S)-ethyl 1-((4-nitrophenoxy)(phenoxy)phosphoryl)pyrrolidine-2-carboxylate. Intermediate (S)-ethyl pyrrolidine-2-carboxylate hydrochloride (1.725 g, 9.602 mmol) was dissolved in CH₂Cl₂ (50 mL). This solution was cooled in an ice bath and phenyl dichlorophosphate (1.242 mL, 8.35 mmol) was added, followed by Et₃N (2.58 mL, 18.37 mmol). The reaction turns into a heterogenous white mixture. The cold bath was removed and the reaction was stirred at room temperature for 1 hour 30 minutes. 4-Nitrophenol (1.1 g, 7.932 mmol) was then added, followed by additional Et₃N (1.17 mL, 8.35 mmol). Reaction progress was monitored by LC/M and after 40 minutes added NMI (0.632 mL, 7.932 mmol) was added. The reaction was stirred for an additional 30 minutes and then diluted with Et₂O. The resulting solids was removed by filtration. The filtrate was concentrated and the intermediate was isolated by silica gel column chromatography (25 g load cartridge, 120 g Combiflash HP Gold Column, eluent ramp from 100% hexanes to 50% EtOAc/hexanes). ¹H NMR (400 MHz, DMSO-d₆) δ 8.35-8.25 (m, 2H), 7.56-7.39 (m, 4H), 7.33-7.21 (m, 3H), 4.35-4.26 (m, 1H), 4.10-3.96 (m, 2H), 3.41-3.33 (m, 2H), 2.18-2.06 (m, 1H), 1.96-1.74 (m, 3H), 1.11 (td, J=7.1, 2.8 Hz, 3H). ³¹P NMR (162 MHz, Methanol-d₄) δ −3.589 (s), −3.688 (s). MS m/z=421.2 [M+1].

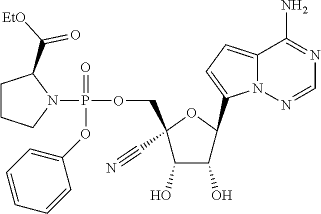 [SMILES: CCOC(=O)[C@@H]1CCCN1P(=O)(OC[C@@]1(C#N)O[C@@H](c2ccc3c(N)ncnn23)[C@H](O)[C@@H]1O)Oc1ccccc1]

(2R)-ethyl 1-((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)pyrrolidine-2-carboxylate. Intermediate 4 (0.05 g, 0.151 mmol) was dissolved in NMP (0.5 mL) and then THF (0.5 mL) was added. This solution was cooled in an ice bath and a 1 M solution of tert-BuMgCl in THF (0.181 mL, 0.181 mmol) was added, causing a white precipitate to form. The cold bath was removed and the reaction was stirred at room temperature for 10 minutes. A solution of intermediate (2S)-ethyl 1-((4-nitrophenoxy)(phenoxy)phosphoryl)pyrrolidine-2-carboxylate (0.089 g, 0.211 mmol) in THF (0.5 mL) was added. The reaction was stirred at room temperature and progress was monitored by LC/MS. After 5 hours the reaction was cooled in an ice bath and quenched by the addition of glacial AcOH (0.026 mL, 0.453 mmol). The ice bath was removed at stirring was continued for 10 minutes at room temperature. Volatiles were removed by evaporation and the product (0.065 g, 70%) was isolated from the residue by HPLC.

The product (0.065 g, 0.106 mmol) from the previous reaction was dissolved in THF (2 mL) and a 12 M aqueous solution of HCl was added drop-wise. The reaction was stirred at room temperature and progress was monitored by LC/MS. After 2 hours 10 minutes the reaction was cooled in an ice bath and the reaction was quenched by the addition of a saturated aqueous NaHCO₃ solution (˜3 mL) followed by the portion-wise addition of solid NaHCO₃ until the pH of the reaction was no longer acidic. The volatile components of the resulting mixture were removed by rotary evaporation and the residue was portioned between minimal water and CH₂Cl₂. The aqueous phase was then further extracted with CH₂Cl₂ and the combined organics were concentrated and the product was isolated from the residue by HPLC. ¹H NMR (400 MHz, Methanol-d₄) δ 7.82 (s, 1H), 7.38-7.29 (m, 2H), 7.29-7.15 (m, 3H), 6.86 (d, J=4.5 Hz, 1H), 6.75 (d, J=4.5 Hz, 1H), 5.51 (d, J=4.7 Hz, 1H), 4.63 (dd, J=5.7, 4.7 Hz, 1H), 4.45 (d, J=5.7 Hz, 1H), 4.37-4.34 (m, 2H), 4.12 (dt, J=8.6, 3.8 Hz, 1H), 4.02 (quart, J=7.1 Hz, 2H), 3.42-3.34 (m, 1H), 3.30-3.24 (m, 1H), 2.02-1.94 (m, 1H), 1.86-1.67 (m, 3H), 1.15 (t, J=7.1 Hz, 3H). ³¹P NMR (162 MHz, Methanol-d₄) δ 1.605 (s), 1.354 (s). MS m/z=572.99 [M+1].

### Example 118. (2S)-cyclobutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

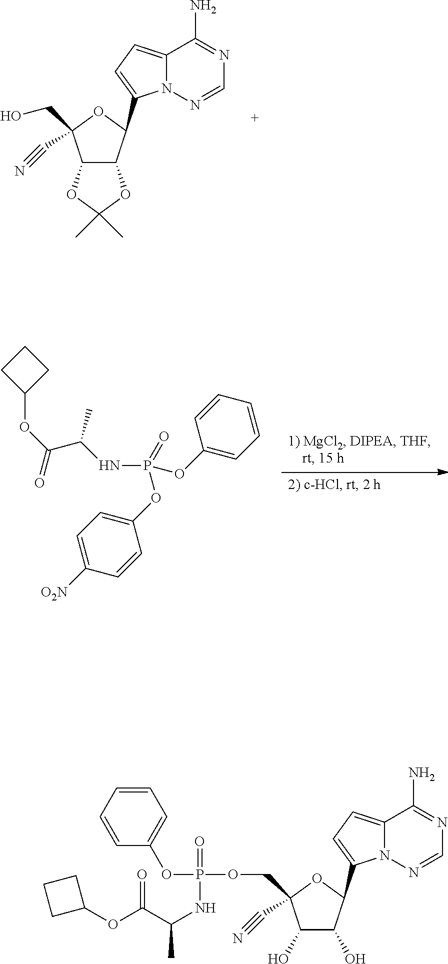

To a mixture of Intermediate 4 (79 mg, 0.24 mmol), Intermediate 47 (150 mg, 0.36 mmol), and MgCl₂ (34 mg, 0.36 mmol) in THF (3 mL) was added N,N-diisopropylethylamine (0.104 mL, 0.60 mmol) dropwise at room temperature. The resulting mixture was stirred at 50° C. for 15 h, diluted with EtOAc, washed with water and brine, dried with sodium sulfate, and concentrated in vacuo. The resulting residue was dissolved in ACN (3 mL) and c-HCl (0.5 mL) was added. The resulting mixture was stirred for 2 h and purified by preparative HPLC (Phenominex Synergi 4 u Hydro-RR 80 Å 150×30 mm column, 10-70% acetonitrile/water gradient) to give the product. ¹H NMR (400 MHz, Methanol-d4) δ 7.70 (m, 1H), 7.26-7.16 (m, 2H), 7.17-7.01 (m, 3H), 6.75 (m, 1H), 6.64 (m, 1H), 5.40 (m, 1H), 4.75 (m, 1H), 4.60-4.47 (m, 1H), 4.47-4.13 (m, 3H), 3.85-3.65 (m, 1H), 2.26-2.03 (m, 2H), 1.99-1.77 (m, 2H), 1.77-1.37 (m, 2H), 1.16 (m, 3H). ³¹P NMR (162 MHz, Methanol-d4) δ 3.27. MS m/z=573 (M+H)⁺.

### Example 119. Separation of Example 2 Diastereomers

Example 2 was separated by chiral preparatory HPLC (Chiralpak IA, 150×4.6 mm, Heptane 80% Ethanol 20%) to afford the diastereomers:

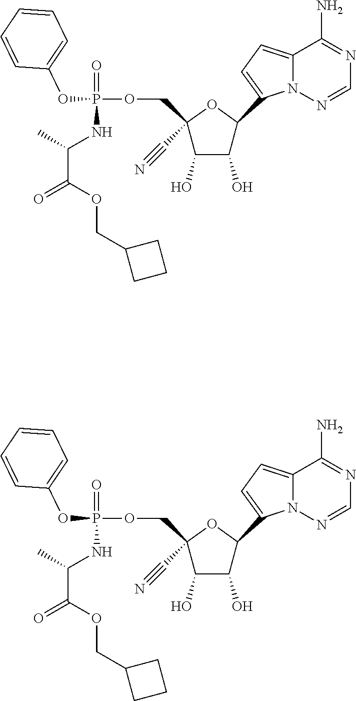 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CCC1 ; C[C@H](N[P@](=O)(OC[C@@]1(C#N)O[C@@H](c2ccc3c(N)ncnn23)[C@H](O)[C@@H]1O)Oc1ccccc1)C(=O)OCC1CCC1]

First eluting diastereomer: ¹H NMR (400 MHz, Methanol-d4) δ 7.78 (s, 1H), 7.34-7.25 (m, 2H), 7.18-7.10 (m, 3H), 6.85 (d, J=4.5 Hz, 1H), 6.73 (d, J=4.5 Hz, 1H), 5.50 (d, J=5.0 Hz, 1H), 4.63 (t, J=5.3 Hz, 1H), 4.55-4.42 (m, 2H), 4.35 (dd, J=10.9, 5.1 Hz, 1H), 4.03 (qd, J=10.9, 6.7 Hz, 2H), 3.88 (dq, J=9.5, 7.1 Hz, 1H), 2.59 (p, J=7.3 Hz, 1H), 2.07-1.94 (m, 2H), 1.94-1.66 (m, 4H), 1.26 (dd, J=7.2, 1.2 Hz, 3H). ³¹P NMR (162 MHz, Methanol-d4) δ 3.26.

### Example 120. Second Eluting Diastereomer

¹H NMR (400 MHz, Methanol-d4) δ 7.80 (s, 1H), 7.38-7.27 (m, 2H), 7.26-7.09 (m, 3H), 6.84 (d, J=4.5 Hz, 1H), 6.73 (d, J=4.5 Hz, 1H), 5.49 (d, J=5.0 Hz, 1H), 4.61 (t, J=5.3 Hz, 1H), 4.46 (d, J=5.6 Hz, 1H), 4.41 (dd, J=10.9, 6.3 Hz, 1H), 4.33 (dd, J=10.9, 5.5 Hz, 1H), 4.00 (dd, J=10.9, 6.8 Hz, 1H), 3.96-3.83 (m, 2H), 2.54 (p, J=7.4 Hz, 1H), 2.04-1.93 (m, 2H), 1.93-1.77 (m, 2H), 1.77-1.61 (m, 2H), 1.26 (dd, J=7.1, 1.1 Hz, 3H). ³¹P NMR (162 MHz, Methanol-d4) δ 3.24.

### Example 121. (2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-2-cyclohexylacetate

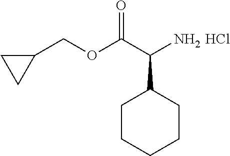 [SMILES: N[C@H](C(=O)OCC1CC1)C1CCCCC1 ; Cl]

(S)-cyclopropylmethyl 2-amino-2-cyclohexylacetate hydrochloride. Took up L-cyclohexylglycine (0.80 g, 5.75 mmol) in cyclopropylmethanol (10 mL) and added chlorotimethylsilane (1.16 mL, 9.16 mmol) in one portion. Placed in a preheated 60° C. oil bath for 16 h. Concentrated and co-evaporated with toluene 5 times in a 60° C. rotary evaporator bath. Placed under high vacuum overnight to afford the intermediate. The material was used as is for the next step. ¹H NMR (400 MHz, DMSO-d₆) δ 8.44 (br s, 2H), 4.40 (br s, 1H), 4.12-3.91 (m, 1H), 3.86 (dd, J=18.9, 6.2 Hz, 2H), 1.88-1.51 (m, 2H), 1.39-0.71 (m, 5H), 0.65-−0.07 (m, 8H).

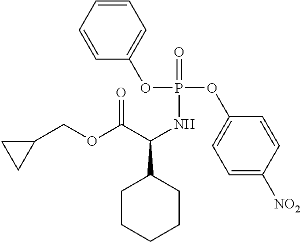 [SMILES: O=C(OCC1CC1)[C@@H](NP(=O)(Oc1ccccc1)Oc1ccc([N+](=O)[O-])cc1)C1CCCCC1]

(2S)-cyclopropylmethyl 2-cyclohexyl-2-(((4-nitrophenoxy)(phenoxy)phosphoryl)amino)acetate. To a solution of (S)-cyclopropylmethyl 2-amino-2-cyclohexylacetate hydrochloride (0.96 g, 4.53 mmol) and phenyl dichlorophosphate (0.68 mL, 4.53 mmol) in dichloromethane (50 mL) was added triethylamine (1.40 mL, 9.92 mmol) at 0° C. under and argon atmosphere. The resulting mixture was allowed to warm to RT and was stirred for 1 h. 4-Nitrophenol (599 mg, 4.31 mmol) and triethylamine (0.69 mL, 4.97 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford impure intermediate. The partially pure material was then purified by reverse phase HPLC without modifier 20-100% ACN in Water to afford the intermediate. ¹H NMR (400 MHz, DMSO-d₆) δ 8.28 (m, 2H), 7.53-7.33 (m, 3H), 7.33-7.05 (m, 3H), 6.57 (dt, J=12.8, 10.5 Hz, 1H), 3.84-3.73 (m, 2H), 3.64 (m, 1H), 1.50 (m, 5H), 1.37-0.67 (m, 8H), 0.44 (m, 2H), 0.19 (m, 2H). ³¹P NMR (162 MHz, DMSO-d₆) δ −0.40 (s), −0.62 (s). LC/MS: t_{R}=2.05 min, MS m/z=489.01 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

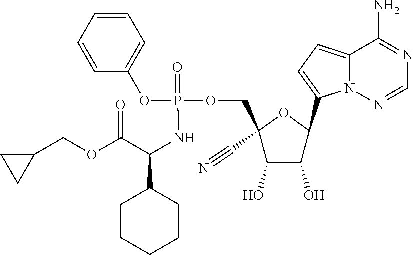 [SMILES: N#C[C@]1(COP(=O)(N[C@H](C(=O)OCC2CC2)C2CCCCC2)Oc2ccccc2)O[C@@H](c2ccc3c(N)ncnn23)[C@H](O)[C@@H]1O]

(2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-2-cyclohexylacetate. To a mixture of Intermediate 4 (43.0 mg, 0.10 mmol), intermediate (2S)-cyclopropylmethyl 2-cyclohexyl-2-(((4-nitrophenoxy)(phenoxy)phosphoryl)amino)acetate (58.42 mg, 0.120 mmol), and magnesium chloride (14.23 mg, 0.149 mmol) was added THF (1.0 mL) at RT. The resulting suspension was warmed to 50° C., and was allowed to stir for 10 min. N,N-Diisopropylethylamine (0.043 mL, 0.249 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.42 min, MS m/z=641.07 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.74 (br s, 2H), 7.31 (td, J=8.6, 7.1 Hz, 2H), 7.21-7.03 (m, 3H), 6.84 (d, J=4.5 Hz, 1H), 6.70 (d, J=4.5 Hz, 1H), 6.09 (d, J=8.31 Hz, 1H), 5.96 (m, 1H), 5.47 (m, 1H), 5.37 (t, J=5.9 Hz, 1H), 4.55-4.37 (m, 1H), 4.37-4.22 (m, 2H), 4.14 (m, 1H), 3.94-3.64 (m, 2H), 3.53 (td, J=10.2, 6.5 Hz, 1H), 1.71-1.31 (m, 6H), 1.21-0.81 (m, 6H), 0.55-0.31 (m, 2H), 0.19 (dd, J=12.1, 5.3 Hz, 2H). ³¹P NMR (162 MHz, DMSO-d₆) δ 3.96 (s), 3.93 (s).

### Example 122. (2S)—(S)-pyrrolidin-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

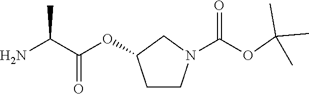 [SMILES: C[C@H](N)C(=O)O[C@H]1CCN(C(=O)OC(C)(C)C)C1]

(S)-tert-butyl 3-(((S)-2-aminopropanoyl)oxy)pyrrolidine-1-carboxylate. Cbz-L-Alanine (446 mg, 2 mmol) was mixed with (S)-Boc-3-pyrrolidinol 374 mg, 2 mmol) and dissolved in anhydrous N,N-dimethylformamide (5 mL). HATU (789 mg, 2.1 mmol) was added in one portion and stirred for 15 mins. Triethylamine (588 μL, 4 mmol) was added in one portion. Reaction was stirred for 16 hrs. Reaction was diluted with ethyl acetate (30 mL) and washed with 5% aqueous citric acid solution (2×15 mL), followed with saturated aqueous sodium bicarbonate solution (2×15 mL) and finally with brine (15 mL). Organic layer was dried over anhydrous sodium sulfate and concentrated under reduced pressure. The crude product was purified via SiO₂ column chromatography (24 g SiO₂ Combiflash HP Gold Column 0-50% ethyl acetate/hexanes). Fractions having the desired product were combined and concentrated under reduced pressure to give an oil which was then dissolved in tetrahydrofuran (15 mL). Degussa 10% palladium on carbon (50 mg) was added and the reaction mixture was stirred under atmospheric hydrogen for 2 hrs. Catalyst was filtered off, and filtrate was concentrated under reduced pressure to give the intermediate. The material was used for next step without purification. ¹H NMR (400 MHz, Chloroform-d) δ 5.30 (s, 1H), 3.64-3.28 (m, 5H), 2.17-1.93 (m, 2H), 1.46 (s, 9H), 1.33 (dd, J=7.0, 2.2 Hz, 3H).

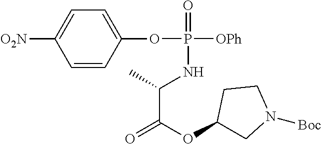 [SMILES: C[C@H](NP(=O)(Oc1ccccc1)Oc1ccc([N+](=O)[O-])cc1)C(=O)O[C@H]1CCN(C(=O)OC(C)(C)C)C1]

(3S)-tert-butyl 3-(((2S)-2-(((4-nitrophenoxy)(phenoxy)phosphoryl)amino)propanoyl) oxy)pyrrolidine-1-carboxylate. Phenyl dichlorophosphate (161 μL, 1.08 mmol) was dissolved in anhydrous dichloromethane (10 mL) and stirred under atmosphere nitrogen in an ice bath. (S)-tert-butyl 3-(((S)-2-aminopropanoyl)oxy)pyrrolidine-1-carboxylate (280 mg, 1.08 mmol) was added in one portion. Triethylamine (332 μL, 2.4 mmol) was added dropwise and stirred for 1 hr. p-Nitrophenol (120 mg, 0.86 mmol) was added and ice bath was removed. Reaction was then stirred for 14 hrs. Reaction was diluted with dichloromethane (20 mL) and washed with water (10 mL), followed with 2% aqueous citric acid solution (10 mL). Organic layer was dried over anhydrous sodium sulfate and concentrated under reduced pressure. The crude product was purified via SiO₂ column chromatography (12 g SiO₂ Combiflash HP Gold Column 0-50% ethyl acetate/hexanes) to afford the intermediate. ¹H NMR (400 MHz, DMSO-d₆) δ 8.28 (m, 2H), 7.53-7.32 (m, 4H), 7.30-7.15 (m, 3H), 6.69 (m, 1H), 5.13 (s, 1H), 4.01-3.92 (m, 1H), 3.52-3.30 (m, 2H), 3.17 (t, J=9.6 Hz, 2H), 2.02 (m, 1H), 1.80 (m, 1H), 1.35 (m, 9H), 1.21 (m, 3H). ³¹P NMR (162 MHz, DMSO-d₆) δ −1.36, −1.45. MS m/z=558.1 [M+Na], 534.2 [M−1].

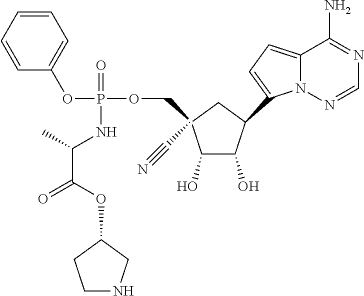

(2S)—(S)-pyrrolidin-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. Intermediate 4 (50 mg, 0.15 mmol) and (3S)-tert-butyl 3-(((2S)-2-(((4-nitrophenoxy)(phenoxy)phosphoryl)amino)propanoyl) oxy)pyrrolidine-1-carboxylate (88 mg, 0.165 mmol) were dissolved in anhydrous tetrahydrofuran (3 mL). Magnesium chloride (21 mg, 0.225 mmol) was added in one portion. Reaction was warmed to 50° C. and stirred for 30 mins. N,N-Diisopropylethylamine (65 μL, 0.375 mmol) was added, and the reaction was stirred for 16 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (300 μL) was added dropwise and stirred in an ice bath for 100 mins. Reaction was diluted with ethyl acetate (30 mL) and cooled in an ice bath. Saturated aqueous sodium bicarbonate solution was added dropwise to give pH of 19. Organic layer was collected, washed with brine (10 mL), dried over anhydrous sodium sulfate and concentrated under reduced pressure. The crude product was purified via prep HPLC (Phenomenex Gemini C₁₈ column, 0-100% acetonitrile/water with 0.1% trifluoroacetic acid as modifier). Fractions having the desired product were combined and freeze dried to give the product. ¹H NMR (400 MHz, Methanol-d₄) δ 8.05-7.96 (m, 1H), 7.43-7.09 (m, 6H), 6.98-6.90 (m, 1H), 5.58-5.50 (m, 1H), 5.47-5.34 (m, 1H), 4.59-4.51 (m, 1H), 4.51-4.31 (m, 3H), 3.97 (m, 1H), 3.56-3.32 (m, 4H), 2.36-2.09 (m, 2H), 1.41-1.29 (m, 3H). ³¹P NMR (162 MHz, Methanol-d₄) δ 3.55, 3.43. MS m/z=588.1 [M+1], 586.0 [M−1].

### Example 123. (2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-2-cyclopentylacetate

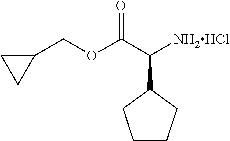

(S)-cyclopropylmethyl 2-amino-2-cyclopentylacetate hydrochloride. Took up L-cyclohexylglycine (0.80 g, 5.75 mmol) in cyclopropylmethanol (10 mL) and added chlorotimethylsilane (1.16 mL, 9.16 mmol) in one portion. Placed in a preheated 60° C. oil bath for 16 h. Concentrated and coevaporated with toluene 5 times in a 60° C. rotary evaporator bath. Placed under high vacuum overnight to afford the intermediate. The material was used as is for the next step. LC/MS: t_{R}=0.61 min, MS m/z=197.95 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

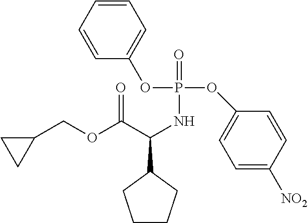 [SMILES: O=C(OCC1CC1)[C@@H](NP(=O)(Oc1ccccc1)Oc1ccc([N+](=O)[O-])cc1)C1CCCC1]

(2S)-cyclopropylmethyl 2-cyclopentyl-2-(((4-nitrophenoxy)(phenoxy)phosphoryl)amino)acetate. To a solution of (S)-2-ethylbutyl 2-amino-2-cyclohexylacetate hydrochloride (1.19 g, 4.80 mmol) and phenyl dichlorophosphate (0.72 mL, 4.80 mmol) in dichloromethane (50 mL) was added triethylamine (1.40 mL, 9.92 mmol) at 0° C. under and argon atmosphere. The resulting mixture was allowed to warm to RT and was stirred for 1 h. 4-Nitrophenol (635 mg, 4.56 mmol) and triethylamine (0.69 mL, 4.97 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford impure intermediate. The partially pure material was then purified by reverse phase HPLC without modifier 20-100% ACN in Water to afford the intermediate. ¹H NMR (400 MHz, DMSO-d₆) δ 8.28 (d, J=9.0 Hz, 2H), 7.58-7.28 (m, 3H), 7.31-7.08 (m, 3H), 6.70 (dt, J=13.0, 10.7 Hz, 1H), 3.87-3.71 (m, 2H), 3.63 (qd, J=9.5, 5.7 Hz, 1H), 2.09 (dt, J=13.7, 6.8 Hz, 1H), 1.64-0.68 (m, 10H), 0.53-0.35 (m, 2H), 0.19 (m, 2H). ³¹P NMR (162 MHz, DMSO-d₆) δ −0.71 (s), −0.97 (s). LC/MS: t_{R}=1.94 min, MS m/z=475.02 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

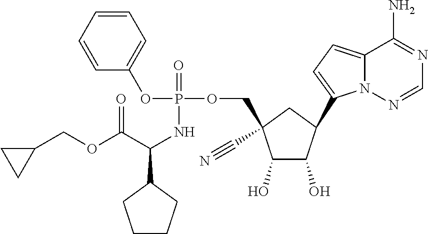 [SMILES: N#C[C@]1(COP(=O)(N[C@H](C(=O)OCC2CC2)C2CCCC2)Oc2ccccc2)C[C@@H](c2ccc3c(N)ncnn23)[C@H](O)[C@@H]1O]

(2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-2-cyclopentylacetate. To a mixture of Intermediate 4 (43.0 mg, 0.10 mmol), intermediate (2S)-cyclopropylmethyl 2-cyclopentyl-2-(((4-nitrophenoxy)(phenoxy)phosphoryl)amino)acetate (64.0 mg, 0.135 mmol), and magnesium chloride (14.23 mg, 0.149 mmol) was added THF (1.0 mL) at RT. The resulting suspension was warmed to 50° C., and was allowed to stir for 10 min. N,N-Diisopropylethylamine (0.043 mL, 0.249 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.37 min, MS m/z=627.07 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.75 (s, 2H), 7.31 (m, 2H), 7.25-7.05 (m, 3H), 6.84 (d, J=4.5 Hz, 1H), 6.71 (d, J=4.5 Hz, 1H), 6.22-5.95 (m, 2H), 5.47 (d, J=5.9 Hz, 1H), 5.36 (t, J=6.0 Hz, 1H), 4.45 (m, 1H), 4.28 (m, 2H), 4.13 (m, 1H), 3.94-3.61 (m, 2H), 3.62-3.40 (m, 1H), 1.71-0.88 (m, 10H), 0.43 (m, 2H), 0.31-0.08 (m, 2H). ³¹P NMR (162 MHz, DMSO-d₆) δ 3.74 (s), 3.64 (s).

### Example 124. (2R)-butyl 1-((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)pyrrolidine-2-carboxylate

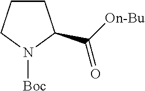

(S)-1-tert-butyl 2-butyl pyrrolidine-1,2-dicarboxylate. The intermediate was prepared in a manner similar to that described for Example 117. ¹H NMR (400 MHz, DMSO-d₆) δ 4.18-3.96 (m, 3H), 3.40-3.26 (m, 2H), 2.26-2.13 (m, 1H), 1.87-1.75 (m, 3H), 1.60-1.48 (m, 2H), 1.39 (s, Boc rotamer #1, 3H), 1.33 (s, Boc rotamer #2, 6H), 1.36-1.28 (m, 2H), 0.88 (td, J=7.4, 2.8 Hz, 3H).

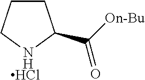

(S)-butyl pyrrolidine-2-carboxylate hydrochloride. The intermediate was prepared in a manner similar to that described for Example 117.

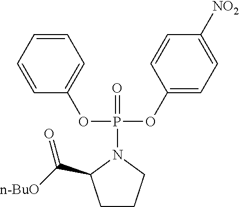

(2S)-butyl 1-((4-nitrophenoxy)(phenoxy)phosphoryl)pyrrolidine-2-carboxylate. The intermediate was prepared in a manner similar to that described for Example 117. ¹H NMR (400 MHz, DMSO-d₆) δ 8.30 (m, 2H), 7.55-7.50 (m, 2H), 7.49-7.21 (m, 5H), 4.36-4.28 (m, 1H), 4.05-3.90 (m, 2H), 3.41-3.33 (m, 2H), 2.18-2.06 (m, 1H), 1.95-1.74 (m, 3H), 1.50-1.40 (m, 2H), 1.30-1.20 (m, 2H), 0.82 (q, J=7.2 Hz, 3H). ³¹P NMR (162 MHz, DMSO-d₆) δ −3.596 (s), −3.726 (s). MS m/z=449.03 [M+1].

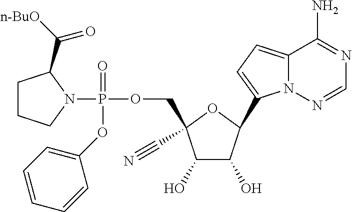

(2R)-butyl 1-((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)pyrrolidine-2-carboxylate. The product was prepared in a manner similar to that described for Example 117. ¹H NMR (400 MHz, Methanol-d₄) δ 7.81 (s, 1H), 7.37-7.14 (m, 5H), 6.86 (d, J=4.5 Hz, 1H), 6.75 (d, J=4.5 Hz, 1H), 5.51 (d, J=4.7 Hz, 1H), 4.62 (dd, J=5.6, 4.8 Hz, 1H), 4.44 (d, J=5.7 Hz, 1H), 4.35 (d, J=7.2 Hz, 2H), 4.16-4.10 (m, 1H), 4.03-3.93 (m, 2H), 3.43-3.35 (m, 1H), 3.30-3.24 (m, 1H), 2.04-1.91 (m, 1H), 1.87-1.69 (m, 3H), 1.56-1.46 (m, 2H), 1.36-1.24 (m, 2H), 0.88 (t, J=7.2 Hz, 3H). ³¹P NMR (162 MHz, Methanol-d₄) δ 1.575 (s), 1.347 (s). MS m/z=601.13 [M+1].

### Example 125. (5-methyl-2-oxo-1,3-dioxol-4-yl)methyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutane-1-carboxylate

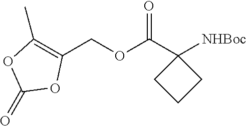 [SMILES: Cc1oc(=O)oc1COC(=O)C1(NC(=O)OC(C)(C)C)CCC1]

(5-methyl-2-oxo-1,3-dioxol-4-yl)methyl 1-((tert-butoxycarbonyl)amino)cyclobutanecarboxylate. The intermediate was prepared in a manner similar to that described for Example 117. ¹H NMR (400 MHz, Methanol-d₄) δ 7.73 (s, rotamer #1, 0.80H), 7.41 (s, rotamer #2, 0.2H), 5.00 (s, rotamer #2, 0.5H), 4.96 (s, rotamer #1, 1.5H), 2.48-2.39 (2H), 2.20-2.05 (5H), 1.95-1.76 (2H), 1.34 (s, rotamer #1, 6.75H), 1.26 (s, rotamer #2, 2.25H).

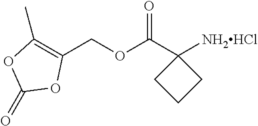

(5-methyl-2-oxo-1,3-dioxol-4-yl)methyl 1-aminocyclobutanecarboxylate hydrochloride. The intermediate was prepared in a manner similar to that described for Example 117.

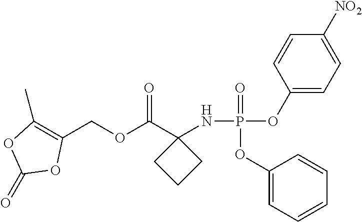 [SMILES: Cc1oc(=O)oc1COC(=O)C1(NP(=O)(Oc2ccccc2)Oc2ccc([N+](=O)[O-])cc2)CCC1]

(5-methyl-2-oxo-1,3-dioxol-4-yl)methyl 1-(((4-nitrophenoxy)(phenoxy)phosphoryl)amino)cyclobutanecarboxylate. The intermediate was prepared in a manner similar to that described for Example 117. ¹H NMR (400 MHz, Methanol-d₄) δ 8.32-8.27 (m, 2H), 7.47-7.37 (m, 4H), 7.25-7.19 (m, 3H), 7.04 (d, J=11.8 Hz, 1H), 4.95 (s, 2H), 2.48-2.42 (m, 2H), 2.29-2.16 (m, 2H), 2.09 (s, 3H), 1.88-1.73 (m, 2H). ³¹P NMR (162 MHz, Methanol-d₄) δ −3.216 (s). MS m/z=504.93 [M+1], 1008.74 [2M+1].

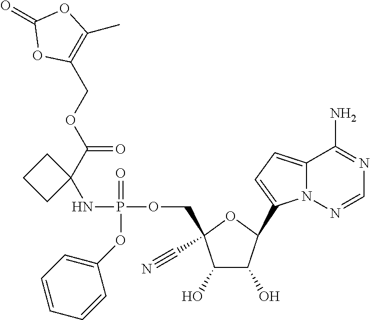 [SMILES: Cc1oc(=O)oc1COC(=O)C1(NP(=O)(OC[C@@]2(C#N)O[C@@H](c3ccc4c(N)ncnn34)[C@H](O)[C@@H]2O)Oc2ccccc2)CCC1]

(5-methyl-2-oxo-1,3-dioxol-4-yl)methyl 1-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)cyclobutane-1-carboxylate. Intermediate 4 (0.054 g, 0.163 mmol), intermediate (5-methyl-2-oxo-1,3-dioxol-4-yl)methyl 1-(((4-nitrophenoxy)(phenoxy)phosphoryl)amino)cyclobutanecarboxylate (0.099 g. 0.196 mmol) and MgCl₂ (0.023 g, 0.244 mmol) were taken up in THF (2.5 mL). The resulting mixture was placed in a 50° C. bath for 10 minutes and then DIPEA (0.071 mL, 0.407 mmol) was added. The reaction was stirred at 50° C. and reaction progress was monitored by LC/MS. After 2 hours 40 minutes the reaction was cooled to room temperature and 12 M HCl (aq) (0.3 mL) was added dropwise. The reaction was stirred at room temperature and reaction progress was monitored by LC/MS. After 35 minutes additional 12 M HCl (aq) (0.15 mL) was added. The reaction was stirred for another 35 minutes. The reaction was cooled in an ice bath and quenched with the addition of a saturated solution of NaHCO₃. The resulting mixture was extracted with CH₂Cl₂ (3×). The combined organic extracts were concentrated and the product was isolated from the residue by HPLC as a mixture of isomers at phosphorous. ¹H NMR (400 MHz, Methanol-d₄, chemical shift with asterisk (*) denotes shift of associated proton(s) on the 2^{nd} isomer present) δ 7.80 (s, 0.6H), 7.77* (s, 0.4H), 7.36-7.12 (m, 5H), 6.87-6.82 (m, 1H), 6.75 (d, J=4.6 Hz, 0.6H), 6.71* (d, J=4.6 Hz, 0.4H), 5.52-5.48 (m, 1H), 4.94 (d, J=2.2 Hz, 0.8H), 4.89* (d, J=2.2 Hz, 1.2H), 4.65 (t, J=5.6 Hz, 0.6H), 4.62* (t, J=5.6 Hz, 0.4H), 4.49 (quart., J=5.5 Hz, 1H), 4.46-4.33 (m, 2H), 2.57-2.42 (m, 2H), 2.31-2.15 (m, 2H), 2.13 (s, 1.2H), 2.10 (s, 1.8H), 1.94-1.80 (m, 2H). ³¹P NMR (162 MHz, Methanol-d₄) δ 1.841 (s), 1.735 (s). MS m/z=657.01 [M+1].

### Example 126. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 3-hydroxy-2,2-dimethylpropanoate

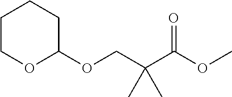

methyl 2,2-dimethyl-3-((tetrahydro-2H-pyran-2-yl)oxy)propanoate. Methyl 2,2-dimethyl-3-hydroxypropionate (1.3 g, 10 mmol) was dissolved in anhydrous dichloromethane (12 mL) and stirred in an ice bath under atmospheric nitrogen. p-Toluene sulfonic acid monohydrate (190 mg, 1 mmol) was added in one portion. 3,4-Dihydro-2H-pyran (1.1 mL, 12 mmol) was added dropwise. Reaction was slowly warmed to room temperature and stirred for 12 hrs. Reaction was diluted with dichloromethane (20 mL) and washed with aqueous sodium bicarbonate solution (2×20 mL). Organic layer was dried over anhydrous sodium sulfate and concentrated under reduced pressure. The crude product was purified via SiO₂ column chromatography (24 g SiO₂ Combiflash HP Gold Column 0-10% ethyl acetate/hexanes) to afford the intermediate. ¹H NMR (400 MHz, Chloroform-d) δ 4.59 (t, J=3.3 Hz, 1H), 3.80 (ddd, J=11.6, 8.9, 2.9 Hz, 1H), 3.73 (d, J=9.2 Hz, 1H), 3.68 (s, 3H), 3.50 (ddt, J=9.2, 5.6, 2.6 Hz, 1H), 3.37 (d, J=9.2 Hz, 1H), 1.78 (m, 1H), 1.70-1.44 (m, 5H), 1.22 (s, 3H), 1.20 (s, 3H).

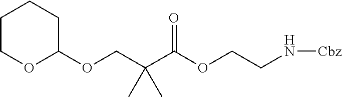 [SMILES: CC(C)(COC1CCCCO1)C(=O)OCCNC(=O)OCc1ccccc1]

2-(((benzyloxy)carbonyl)amino)ethyl 2,2-dimethyl-3-((tetrahydro-2H-pyran-2-yl)oxy) propanoate. methyl 2,2-dimethyl-3-((tetrahydro-2H-pyran-2-yl)oxy)propanoate (1.5 g, 6.9 mmol) was dissolved in anhydrous tetrahydrofuran (15 mL). Sodium hydroxide (276 mg, 6.9 mmol) was dissolved in water (10 mL) and added to the reaction and stirred for 16 hrs. Methanol (10 mL) was added. Reaction was stirred for 16 hrs. Reaction was washed with hexane (100 mL) and then acidified with 5% aqueous citric acid solution to pH 4 and extracted with ethyl acetate (2×30 mL). Ethyl acetate extracts were combined and dried over anhydrous sodium sulfate and concentrated under reduced pressure to give an oil which was then dissolved in anhydrous N, N-dimethylformamide (7 mL) and stirred at room temperature. N-Cbz-aminoethanol (1.35 g, 6.9 mmol) was added in one portion. HATU (2.88 g, 7.6 mmol) was added and stirred for 5 mins. Triethylamine (1.9 mL, 13.8 mmol) was added and stirred for 10 mins. 4-(Dimethylamino)pyridine (84 mg, 0.69 mmol) was then added. Reaction was stirred for 1 hr. More 4-(Dimethylamino)pyridine (340 mg) was added and stirred for 2 hrs. More 4-(Dimethylamino)pyridine (310 mg) was added and stirred for 16 hrs. Reaction was diluted with ethyl acetate (35 mL) and washed with water (2×20 mL) and then with brine (20 mL). Organic layer was dried over anhydrous sodium sulfate and concentrated under reduced pressure. The crude product was purified via SiO₂ column chromatography (24 g SiO₂ Combiflash HP Gold Column 0-30% ethyl acetate/hexanes) to afford the intermediate. ¹H NMR (400 MHz, Chloroform-d) δ 7.41-7.27 (m, 5H), 5.78 (bs, 1H), 5.21-4.98 (m, 2H), 4.43 (m, 1H), 4.40-4.28 (m, 1H), 4.03 (ddd, J=11.0, 7.1, 3.4 Hz, 1H), 3.91 (d, J=8.8 Hz, 1H), 3.81 (m, 1H), 3.55-3.33 (m, 3H), 3.27 (d, J=8.7 Hz, 1H), 1.66 (m, 2H), 1.41 (m, 4H), 1.19 (d, J=9.8 Hz, 6H).

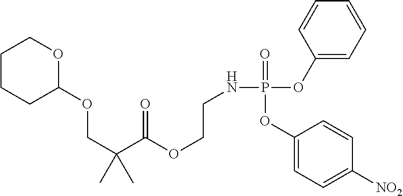 [SMILES: CC(C)(COC1CCCCO1)C(=O)OCCNP(=O)(Oc1ccccc1)Oc1ccc([N+](=O)[O-])cc1]

2-(((4-nitrophenoxy)(phenoxy)phosphoryl)amino)ethyl 2,2-dimethyl-3-((tetrahydro-2H-pyran-2-yl)oxy)propanoate. 2-(((benzyloxy)carbonyl)amino)ethyl 2,2-dimethyl-3-((tetrahydro-2H-pyran-2-yl)oxy) propanoate (1.3 g, 3.4 mmol) was dissolved in tetrahydrofuran (20 mL). Degussa type 10% Palladium on carbon (50 mg) was added and the reaction was stirred under atmospheric hydrogen for 20 hrs. Catalyst was filtered off and filtrate was concentrated under reduced pressure and the resulting crude product was used for next reaction without purification. Phenyl dichlorophosphate (510 μL, 3.4 mmol) was dissolved in anhydrous dichloromethane (15 mL) and stirred under atmosphere nitrogen in an ice bath. Above prepared crude product was dissolved in anhydrous dichloromethane (5 mL) and added dropwise. Triethylamine (1.4 mL, 7.5 mmol) was added dropwise and stirred for 1 hr. p-Nitrophenol (378 mg, 2.72 mmol) was added and ice bath was removed. Reaction was then stirred for 2 hrs. More p-nitrophenol (40 mg) was added and stirred for 1 hr. Reaction was diluted with dichloromethane (20 mL) and washed with 1% aqueous citric acid solution (2×20 mL). Organic layer was dried over anhydrous sodium sulfate and concentrated under reduced pressure. The crude product was purified via SiO₂ column chromatography (24 g SiO₂ Combiflash HP Gold Column 0-30% ethyl acetate/hexanes) to afford the product. ¹H NMR (400 MHz, Chloroform-d) δ 8.23 (d, J=9.0, 2H), 7.48-7.30 (m, 4H), 7.30-7.14 (m, 3H), 4.59-4.27 (m, 3H), 3.95 (m, 3H), 3.46 (m, 1H), 3.41-3.29 (m, 2H), 3.26 (dd, J=8.7, 4.2 Hz, 1H), 1.85-1.34 (m, 6H), 1.24 (s, 3H), 1.16 (s, 3H). ³¹P NMR (162 MHz, Chloroform-d) δ −0.42, −0.56. MS m/z=521.1 [M−1].

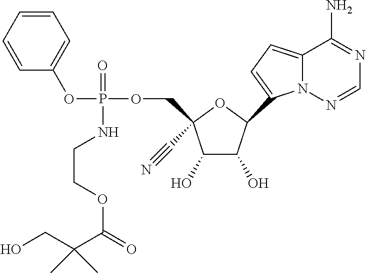 [SMILES: CC(C)(CO)C(=O)OCCNP(=O)(OC[C@@]1(C#N)O[C@@H](c2ccc3c(N)ncnn23)[C@H](O)[C@@H]1O)Oc1ccccc1]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 3-hydroxy-2,2-dimethylpropanoate. Intermediate 4 (50 mg, 0.15 mmol) and 2-(((4-nitrophenoxy)(phenoxy)phosphoryl)amino)ethyl 2,2-dimethyl-3-((tetrahydro-2H-pyran-2-yl)oxy)propanoate (94 mg, 0.18 mmol) were dissolved in anhydrous tetrahydrofuran (3 mL). Magnesium chloride (21 mg, 0.225 mmol) was added in one portion. Reaction was warmed to 50° C. and stirred for 20 mins. N,N-Diisopropylethylamine (65 μL, 0.375 mmol) was added, and the reaction was stirred for 20 hrs at 50° C. Reaction was cooled to room temperature, diluted with ethyl acetate (30 mL) and washed with 2% aqueous sodium carbonate solution (3×20 mL) and then brine (20 mL). Dried over anhydrous sodium sulfate and concentrated under reduced pressure. Residue was dissolved in acetonitrile (2 mL) and stirred in an ice bath. 12 M hydrochloric acid (200 μL) was added dropwise and stirred in an ice bath for 2 hrs. Reaction was diluted with ethyl acetate (30 mL) and cooled in an ice bath. Saturated aqueous sodium bicarbonate solution was added dropwise to give pH of 9. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-3-8% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.82 (m, 1H), 7.28 (m, 5H), 6.88 (m, 1H), 6.76 (m, 1H), 5.54 (m, 1H), 4.66 (m, 1H), 4.52 (t, J=5.1 Hz, 1H), 4.49-4.30 (m, 2H), 4.05 (m, 2H), 3.53 (d, J=10.0 Hz, 2H), 3.18 (dt, J=11.7, 5.6 Hz, 2H), 1.15 (s, 3H), 1.13 (s, 3H). ³¹P NMR (162 MHz, Methanol-d₄) δ 5.31, 5.14. MS m/z=591.0 [M+1], 589.1 [M−1].

### Example 127. (2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-4-methylpentanoate

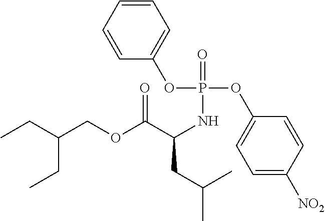 [SMILES: CCC(CC)COC(=O)[C@H](CC(C)C)NP(=O)(Oc1ccccc1)Oc1ccc([N+](=O)[O-])cc1]

(2S)-2-ethylbutyl 4-methyl-2-(((4-nitrophenoxy)(phenoxy)phosphoryl)amino)pentanoate. To a solution of Intermediate 13 (0.76 g, 3.02 mmol) and phenyl dichlorophosphate (0.45 mL, 3.02 mmol) in dichloromethane (20 mL) was added triethylamine (0.87 mL, 6.23 mmol) at 0° C. under and argon atmosphere. The resulting mixture was allowed to warm to RT and was stirred for 1 h. 4-Nitrophenol (399 mg, 2.87 mmol) and triethylamine (0.44 mL, 3.11 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford the intermediate. LC/MS: t_{R}=2.19 min, MS m/z=493.00 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 8.28 (m, 2H), 7.52-7.29 (m, 3H), 7.28-7.09 (m, 2H), 6.66 (m, 1H), 3.93-3.70 (m, 2H), 1.58-1.31 (m, 6H), 1.31-1.13 (m, 5H), 0.86-0.60 (m, 12H). ³¹P NMR (162 MHz, DMSO-d₆) δ −0.87 (s), −1.24 (s).

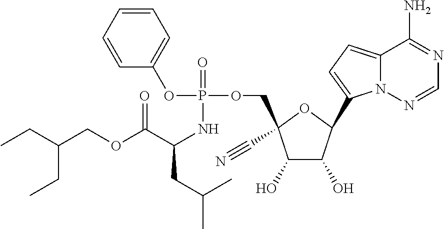 [SMILES: CCC(CC)COC(=O)[C@H](CC(C)C)NP(=O)(OC[C@@]1(C#N)O[C@@H](c2ccc3c(N)ncnn23)[C@H](O)[C@@H]1O)Oc1ccccc1]

To a mixture of Intermediate 4 (38.0 mg, 0.09 mmol), (2S)-2-ethylbutyl 4-methyl-2-(((4-nitrophenoxy)(phenoxy)phosphoryl)amino)pentanoate (52.0 mg, 0.106 mmol), and magnesium chloride (12.58 mg, 0.132 mmol) was added THF (1.0 mL) at RT. The resulting suspension was warmed to 50° C., and was allowed to stir for 10 min. N,N-Diisopropylethylamine (0.038 mL, 0.220 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.53 min, MS m/z=645.10 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.74 (s, 2H), 7.39-7.23 (m, 2H), 7.23-7.06 (m, 3H), 6.84 (d, J=4.5 Hz, 1H), 6.70 (d, J=4.5 Hz, 1H), 6.19-5.92 (m, 2H), 5.48 (d, J=5.8 Hz, 1H), 5.37 (t, J=6.7 Hz, 1H), 4.45 (m, 1H), 4.36-4.14 (m, 2H), 3.94-3.59 (m, 3H), 1.52-1.30 (m, 4H), 1.30-1.13 (m, 5H), 0.89-0.65 (m, 12H). ³¹P NMR (162 MHz, DMSO-d₆) δ 3.61 (s), 3.48 (s).

Separation of the (S) and (R) Diastereomers. The product was purified via chiral preparatory HPLC (Chiralpak IA, 150×4.6 mm, Heptane 80% Ethanol 20%) to afford the diastereomers:

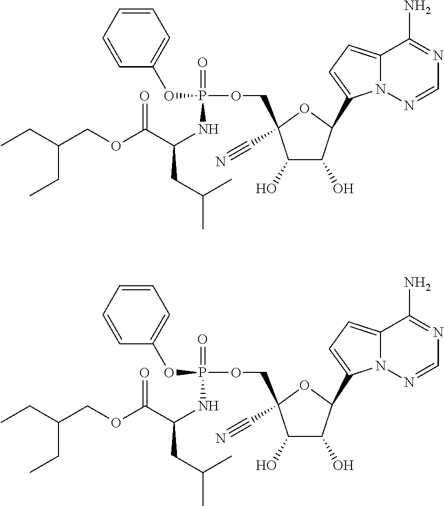 [SMILES: CCC(CC)COC(=O)[C@H](CC(C)C)N[P@@](=O)(OC[C@@]1(C#N)O[C@@H](c2ccc3c(N)ncnn23)[C@H](O)[C@@H]1O)Oc1ccccc1 ; CCC(CC)COC(=O)[C@H](CC(C)C)N[P@](=O)(OC[C@@]1(C#N)O[C@@H](c2ccc3c(N)ncnn23)[C@H](O)[C@@H]1O)Oc1ccccc1]

### Example 128. First Eluting Diastereomer

¹H NMR (400 MHz, Methanol-d₄) δ 7.78 (s, 1H), 7.28 (dd, J=8.6, 7.1 Hz, 2H), 7.20-7.08 (m, 3H), 6.84 (d, J=4.5 Hz, 1H), 6.73 (d, J=4.5 Hz, 1H), 5.49 (d, J=5.7 Hz, 2H), 4.61 (t, J=5.4 Hz, 1H), 4.48 (dd, J=11.3, 5.6 Hz, 2H), 4.36 (dd, J=10.9, 5.5 Hz, 1H), 4.00 (d, J=5.7 Hz, 1H), 3.93-3.82 (m, 1H), 1.63-1.42 (m, 3H), 1.41-1.08 (m, 10H), 1.00-0.68 (m, 11H). ³¹P NMR (162 MHz, CD₃OD) δ 3.52 (s).

### Example 129. Second Eluting Diastereomer

¹H NMR (400 MHz, Methanol-d₄) δ 7.79 (s, 1H), 7.32 (dd, J=8.6, 7.2 Hz, 2H), 7.27-7.10 (m, 3H), 6.84 (d, J=4.5 Hz, 1H), 6.73 (d, J=4.5 Hz, 1H), 5.60-5.37 (m, 2H), 4.60 (t, J=5.3 Hz, 1H), 4.43 (dd, J=16.8, 5.9 Hz, 2H), 4.31 (dd, J=10.9, 5.2 Hz, 1H), 4.05-3.76 (m, 3H), 1.67 (dp, J=13.4, 6.7 Hz, 3H), 1.56-1.15 (m, 10H), 0.93-0.70 (m, 11H). ³¹P NMR (162 MHz, CD₃OD) δ 3.38 (s).

### Example 130. (2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-4-methylpentanoate

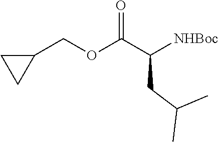 [SMILES: CC(C)C[C@H](NC(=O)OC(C)(C)C)C(=O)OCC1CC1]

(S)-cyclopropylmethyl 2-((tert-butoxycarbonyl)amino)-4-methylpentanoate. Took up cyclopropylmethyl (tert-butoxycarbonyl)-L-leucinate (1.85 g, 7.99 mmol) in acetonitrile (20 mL) and added cyclopropylmethanol (3.16 mL, 39.99 mmol) followed by EDCI (1.49 g, 9.60 mmol) and DMAP (1.47 g, 12.0 mmol) in one portion. Allowed to stir at room temperature overnight. Concentrated and diluted with CH₂Cl₂. Purified by silica gel chromatography 0-40% EtOAc/Hex to afford the intermediate. ¹H NMR (400 MHz, DMSO-d₆) δ 6.93 (d, J=8.7 Hz, 1H), 3.74-3.58 (m, 3H), 1.44-1.09 (m, 12H), 0.83 (m, 1H), 0.64 (m, 6H), 0.25 (m, 2H), 0.06 (m, 2H).

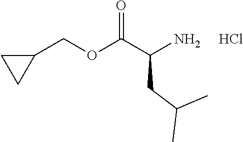

(S)-cyclopropylmethyl 2-amino-4-methylpentanoate hydrochloride. Took up (S)-cyclopropylmethyl 2-((tert-butoxycarbonyl)amino)-4-methylpentanoate in CH₂Cl₂ (15 mL) and 4 N HCl in dioxane (15 mL, 40 mmol). Stirred at ambient temperature for 1 h. Concentrated under reduced pressure and co-evaporated with diethyl ether. Placed under high vacuum for 1 h and the intermediate was used as is without purification for the next step.

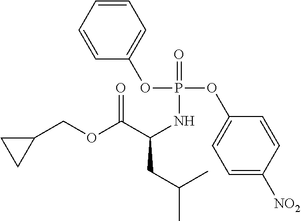 [SMILES: CC(C)C[C@H](NP(=O)(Oc1ccccc1)Oc1ccc([N+](=O)[O-])cc1)C(=O)OCC1CC1]

(2S)-cyclopropylmethyl 4-methyl-2-(((4-nitrophenoxy)(phenoxy)phosphoryl)amino)pentanoate. To a solution of (S)-cyclopropylmethyl 2-amino-4-methylpentanoate hydrochloride (1.29 g, 5.82 mmol) and phenyl dichlorophosphate (0.87 mL, 5.82 mmol) in dichloromethane (30 mL) was added triethylamine (1.68 mL, 12.03 mmol) at 0° C. under and argon atmosphere. The resulting mixture was allowed to warm to RT and was stirred for 1 h. 4-Nitrophenol (769 mg, 5.53 mmol) and triethylamine (0.44 mL, 6.01 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford the intermediate. ¹H NMR (400 MHz, DMSO-d₆) δ 8.28 (m, 2H), 7.59-7.31 (m, 5H), 7.33-7.09 (m, 2H), 6.67 (m, 1H), 3.80 (m, 2H), 1.69-1.29 (m, 3H), 0.99 (m, 1H), 0.86-0.62 (m, 6H), 0.44 (ddt, J=7.0, 5.7, 4.1 Hz, 2H), 0.19 (dq, J=6.7, 4.5, 3.5 Hz, 2H). ³¹P NMR (162 MHz, DMSO-d₆) δ −0.85 (s), −1.15 (s). LC/MS: t_{R}=1.97 min, MS m/z=463.01 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

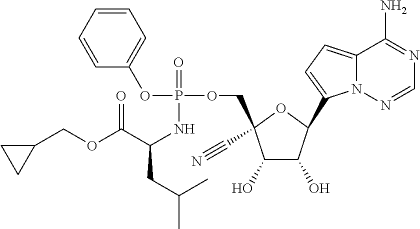 [SMILES: CC(C)C[C@H](NP(=O)(OC[C@@]1(C#N)O[C@@H](c2ccc3c(N)ncnn23)[C@H](O)[C@@H]1O)Oc1ccccc1)C(=O)OCC1CC1]

(2S)-cyclopropylmethyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-4-methylpentanoate. To a mixture of Intermediate 4 (52.0 mg, 0.12 mmol), intermediate (2S)-cyclopropylmethyl 4-methyl-2-(((4-nitrophenoxy)(phenoxy)phosphoryl)amino)pentanoate (67 mg, 0.145 mmol), and magnesium chloride (17.21 mg, 0.18 mmol) was added THF (1.0 mL) at RT. The resulting suspension was warmed to 50° C., and was allowed to stir for 10 min. N,N-Diisopropylethylamine (0.052 mL, 0.220 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.37 min, MS m/z=615.07 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.75 (s, 2H), 7.41-7.23 (m, 2H), 7.24-7.03 (m, 3H), 6.84 (d, J=4.5 Hz, 1H), 6.71 (d, J=4.5 Hz, 1H), 6.31-5.93 (m, 2H), 5.47 (d, J=5.8 Hz, 1H), 5.37 (m, 1H), 4.45 (m, 1H), 4.36-4.16 (m, 2H), 3.96-3.60 (m, 3H), 1.56-1.31 (m, 2H), 1.08-0.87 (m, 2H), 0.93-0.54 (m, 6H), 0.43 (m, 2H), 0.33-0.03 (m, 2H). ³¹P NMR (162 MHz, DMSO-d₆) δ 3.71 (s), 3.43 (s).

### Example 131. (2S)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 2-amino-3-methylbutanoate

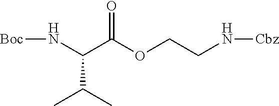 [SMILES: CC(C)[C@H](NC(=O)OC(C)(C)C)C(=O)OCCNC(=O)OCc1ccccc1]

(S)-2-(((benzyloxy)carbonyl)amino)ethyl 2-((tert-butoxycarbonyl)amino)-3-methyl butanoate. Boc-L-Valine (435 mg, 2 mmol) and Cbz-aminoethanol (390 mg, 2 mmol) were mixed and dissolved anhydrous dichloromethane (15 mL). N-Ethyl-N′-(3-Dimethylaminopropyl) carbodiimide hydrochloride (422 mg, 2.2 mmol) was added. Triethylamine (420 μL, 3 mmol) and 4-(Dimethylamino)pyridine (244 mg, 0.2 mmol) were added and the reaction was stirred for 16 hrs. Reaction was diluted with dichloromethane (20 mL) and washed with 2% aqueous citric acid solution (20 mL). Organic layer was dried over anhydrous sodium sulfate and concentrated under reduced pressure. The crude product was purified via SiO₂ column chromatography (12 g SiO₂ Combiflash HP Gold Column 0-30% ethyl acetate/hexanes) to afford the intermediate. ¹H NMR (400 MHz, Chloroform-d) δ 7.39 (m, 5H), 5.17 (m, 2H), 5.00 (d, J=8.4 Hz, 1H), 4.41-4.08 (m, 3H), 3.52 (m, 2H), 2.25-2.09 (m, 1H), 1.46 (s, 9H), 1.00 (d, J=6.8 Hz, 3H), 0.93 (d, J=6.8 Hz, 3H).

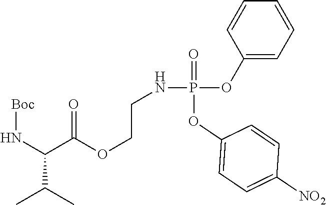 [SMILES: CC(C)[C@H](NC(=O)OC(C)(C)C)C(=O)OCCNP(=O)(Oc1ccccc1)Oc1ccc([N+](=O)[O-])cc1]

(2S)-2-(((4-nitrophenoxy)(phenoxy)phosphoryl)amino)ethyl 2-((tert-butoxycarbonyl) amino)-3-methylbutanoate. (S)-2-(((benzyloxy)carbonyl)amino)ethyl 2-((tert-butoxycarbonyl)amino)-3-methyl butanoate (378 mg, 0.958 mmol) was dissolved in tetrahydrofuran (20 mL). Degussa type 10% Palladium on carbon (50 mg) was added and the reaction was stirred under atmospheric hydrogen for 20 hrs. Catalyst was filtered off and filtrate was concentrated under reduced pressure and the resulting product was used for next reaction without purification. Phenyl dichlorophosphate (143 μL, 0.958 mmol) was dissolved in anhydrous dichloromethane (10 mL) and stirred under atmosphere nitrogen in an ice bath. Above prepared oil was dissolved in anhydrous dichloromethane (5 mL) and added dropwise. Triethylamine (300 μL, 2.1 mmol) was added dropwise and stirred for 1 hr. p-Nitrophenol (107 mg, 0.766 mmol) was added and ice bath was removed. Reaction was then stirred for 2 hrs. Reaction was diluted with dichloromethane (10 mL) and washed with water (3×20 mL). Organic layer was dried over anhydrous sodium sulfate and concentrated under reduced pressure. The crude product was purified via SiO₂ column chromatography (12 g SiO₂ Combiflash HP Gold Column 0-50% ethyl acetate/hexanes) to afford the intermediate. ¹H NMR (400 MHz, Chloroform-d) δ 8.27 (d, J=8.7 Hz, 2H), 7.50-7.33 (m, 4H), 7.25 (m, 3H), 4.98 (m, 1H), 4.41-4.24 (m, 1H), 4.14 (m, 2H), 3.87 (m, 1H), 3.43 (m, 2H), 2.08 (m, 1H), 1.47 (s, 9H), 0.99 (d, J=6.8 Hz, 3H), 0.94 (d, J=6.9 Hz, 3H). ³¹P NMR (162 MHz, Chloroform-d) δ −1.02, −1.06. MS m/z=560.0 [M+Na], 536.0 [M−1].

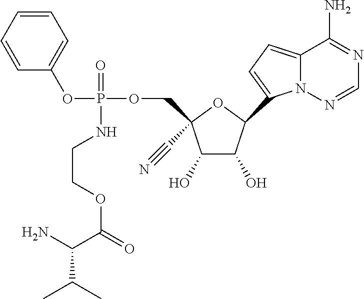 [SMILES: CC(C)[C@H](N)C(=O)OCCNP(=O)(OC[C@@]1(C#N)O[C@@H](c2ccc3c(N)ncnn23)[C@H](O)[C@@H]1O)Oc1ccccc1]

(2S)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 2-amino-3-methylbutanoate. Intermediate 4 (50 mg, 0.15 mmol) and (2S)-2-(((4-nitrophenoxy)(phenoxy)phosphoryl)amino)ethyl 2-((tert-butoxycarbonyl) amino)-3-methylbutanoate (97 mg, 0.18 mmol) were dissolved in anhydrous tetrahydrofuran (3 mL). Magnesium chloride (21 mg, 0.225 mmol) was added in one portion. Reaction was warmed to 50° C. and stirred for 20 mins. N,N-Diisopropylethylamine (65 μL, 0.375 mmol) was added, and the reaction was stirred for 20 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (1.5 mL) and stirred in an ice bath. 12 M hydrochloric acid (250 μL) was added dropwise and stirred in an ice bath for 75 mins. Reaction was diluted with ethyl acetate (30 mL) and cooled in an ice bath. Saturated aqueous sodium bicarbonate solution was added dropwise to give pH of 9. Organic layer was collected, washed with brine (10 mL), dried over anhydrous sodium sulfate and concentrated under reduced pressure. The crude product was purified via SiO₂ column chromatography (4 g SiO₂ Combiflash HP Gold Column 0-10-20% methanol/dichloromethane). Fractions having the desired product were combined and concentrated under reduced pressure. Residue was dissolved in acetonitrile and water and freeze dried to give the product. ¹H NMR (400 MHz, Methanol-d₄) δ 7.79 (m, 1H), 7.38-7.09 (m, 5H), 6.88-6.80 (m, 1H), 6.73 (m, 1H), 5.51 (d, J=4.9 Hz, 1H), 4.63 (q, J=5.0 Hz, 1H), 4.49 (dd, J=7.1, 5.6 Hz, 1H), 4.46-4.27 (m, 2H), 4.06 (m, 2H), 3.29-3.24 (m, 1H), 3.17 (m, 2H), 1.98 (m, 1H), 0.89 (m, 6H). ³¹P NMR (162 MHz, Methanol-d₄) δ 5.21, 5.06. MS m/z=590.1 [M+1], 588.0 [M−1].

### Example 132. (2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-3-cyclopropylpropanoate

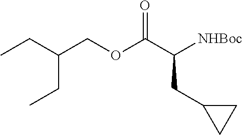 [SMILES: CCC(CC)COC(=O)[C@H](CC1CC1)NC(=O)OC(C)(C)C]

(S)-2-ethylbutyl 2-((tert-butoxycarbonyl)amino)-3-cyclopropylpropanoate. Took up 2-ethylbutanol (1.04 g, 4.54 mmol) in acetonitrile (10 mL) and added 2-ethyl-1-butanol (2.78 mL, 22.68 mmol) followed by EDCI (845 mg, 5.44 mmol) and DMAP (831 mg, 6.80 mmol) in one portion. Allowed to stir at room temperature overnight. Concentrated and diluted with CH₂Cl₂. Purified by silica gel chromatography 0-40% EtOAc/Hex to afford the intermediate. ¹H NMR (400 MHz, DMSO-d₆) δ 7.22 (d, J=8.4 Hz, 1H), 3.99-3.87 (m, 3H), 1.64-1.23 (m, 16H), 0.87-0.76 (m, 7H), 0.41 (m, 2H), 0.15 (m, 1H), 0.01 (m, 1H).

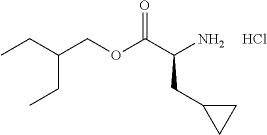

(S)-2-ethylbutyl 2-amino-3-cyclopropylpropanoate hydrochloride. Took up (S)-2-ethylbutyl 2-((tert-butoxycarbonyl)amino)-3-cyclopropylpropanoate (1.03 g, 3.29 mmol) in CH₂Cl₂ (15 mL) and 4 N HCl in dioxane (15 mL, 40 mmol). Stirred at ambient temperature for 1 h. Concentrated under reduced pressure and co-evaporated with diethyl ether. Placed under high vacuum for 1 h and the intermediate was used as is without purification for the next step. ¹H NMR (400 MHz, DMSO-d₆) δ 8.57 (s, 3H), 4.14-3.98 (m, 3H), 1.79 (dt, J=14.4, 6.5 Hz, 1H), 1.70-1.45 (m, 1H), 1.40-1.28 (m, 2H), 1.31-1.20 (m, 1H), 1.23 (s, 1H), 0.85 (t, J=7.5 Hz, 6H), 0.84-0.70 (m, 1H), 0.49-0.39 (m, 2H), 0.14-0.06 (m, 2H).

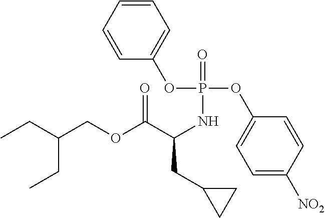 [SMILES: CCC(CC)COC(=O)[C@H](CC1CC1)NP(=O)(Oc1ccccc1)Oc1ccc([N+](=O)[O-])cc1]

(2S)-2-ethylbutyl 3-cyclopropyl-2-(((4-nitrophenoxy)(phenoxy)phosphoryl)amino)propanoate. To a solution of (S)-2-ethylbutyl 2-amino-3-cyclopropylpropanoate hydrochloride (0.87 g, 3.48 mmol) and phenyl dichlorophosphate (0.52 mL, 3.49 mmol) in dichloromethane (20 mL) was added triethylamine (1.0 mL, 7.20 mmol) at 0° C. under argon atmosphere. The resulting mixture was allowed to warm to RT and was stirred for 1 h. 4-Nitrophenol (460 mg, 3.30 mmol) and triethylamine (0.52 mL, 3.60 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford the intermediate. ¹H NMR (400 MHz, DMSO-d₆) δ 8.28 (m, 2H), 7.60-7.32 (m, 4H), 7.32-7.08 (m, 3H), 6.82-6.52 (m, 1H), 3.87 (m, 3H), 1.65-1.30 (m, 3H), 1.30-1.18 (m, 4H), 0.97-0.70 (m, 6H), 0.63 (d, J=6.5 Hz, 1H), 0.27 (m, 2H), −0.07 (q, J=3.8 Hz, 2H). ³¹P NMR (162 MHz, DMSO-d₆) δ −1.09 (s), −1.35 (s). LC/MS: t_{R}=2.13 min, MS m/z=490.99 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

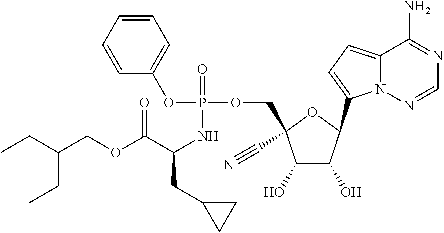 [SMILES: CCC(CC)COC(=O)[C@H](CC1CC1)NP(=O)(OC[C@@]1(C#N)O[C@@H](c2ccc3c(N)ncnn23)[C@H](O)[C@@H]1O)Oc1ccccc1]

(2S)-2-ethylbutyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)-3-cyclopropylpropanoate. To a mixture of Intermediate 4 (52.0 mg, 0.12 mmol), intermediate (2S)-2-ethylbutyl 3-cyclopropyl-2-(((4-nitrophenoxy)(phenoxy)phosphoryl)amino)propanoate (71 mg, 0.145 mmol), and magnesium chloride (17.21 mg, 0.18 mmol) was added THF (1.0 mL) at RT. The resulting suspension was warmed to 50° C., and was allowed to stir for 10 min. N,N-Diisopropylethylamine (0.052 mL, 0.220 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.48 min, MS m/z=643.09 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.83 (s, 1H), 7.74 (s, 2H), 7.31 (m, 2H), 7.24-7.04 (m, 3H), 6.83 (d, J=4.5 Hz, 1H), 6.70 (d, J=4.5 Hz, 1H), 6.27-5.97 (m, 2H), 5.48 (m, 1H), 5.37 (m, 1H), 4.44 (m, 1H), 4.37-4.01 (m, 3H), 3.95-3.65 (m, 3H), 1.64-1.30 (m, 3H), 1.24 (m, 4H), 0.90-0.69 (m, 6H), 0.69-0.55 (m, 1H), 0.28 (m, 2H) −0.04 (m, 2H). ³¹P NMR (162 MHz, DMSO-d₆) δ 3.51 (s), 3.35 (s).

### Example 133. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl L-alaninate

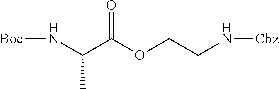 [SMILES: C[C@H](NC(=O)OC(C)(C)C)C(=O)OCCNC(=O)OCc1ccccc1]

(S)-2-(((benzyloxy)carbonyl)amino)ethyl 2-((tert-butoxycarbonyl)amino)propanoate. Boc-L-Alanine (567 mg, 3 mmol) was dissolved anhydrous acetonitrile (15 mL). N-Ethyl-N′-(3-Dimethylaminopropyl)carbodiimide hydrochloride (633 mg, 3.3 mmol) was added and stirred for 20 mins. Cbz-aminoethanol (586 mg, 3 mmol) was added in one portion. 4-(Dimethylamino)pyridine (403 mg, 3.3 mmol) was added and the reaction was stirred for 16 hrs. Reaction was diluted with ethyl acetate (20 mL) and washed with 5% aqueous citric acid solution (20 mL) followed with brine (20 mL). Organic layer was dried over anhydrous sodium sulfate and concentrated under reduced pressure. The crude product was purified via SiO₂ column chromatography (12 g SiO₂ Combiflash HP Gold Column 0-50% ethyl acetate/hexanes) to afford the intermediate. ¹H NMR (400 MHz, Chloroform-d) δ 7.35 (m, 5H), 5.10 (m, 3H), 4.96 (s, 1H), 4.23 (m, 3H), 3.48 (m, 2H), 1.42 (s, 9H), 1.36 (d, J=7.2 Hz, 3H).

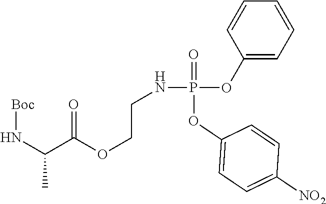 [SMILES: C[C@H](NC(=O)OC(C)(C)C)C(=O)OCCNP(=O)(Oc1ccccc1)Oc1ccc([N+](=O)[O-])cc1]

(2S)-2-(((4-nitrophenoxy)(phenoxy)phosphoryl)amino)ethyl 2-((tert-butoxycarbonyl) amino)propanoate. (S)-2-(((benzyloxy)carbonyl)amino)ethyl 2-((tert-butoxycarbonyl)amino)propanoate (818 mg, 2.23 mmol) was dissolved in tetrahydrofuran (50 mL). Degussa type 10% Palladium on carbon (100 mg) was added and the reaction was stirred under atmospheric hydrogen for 4 hrs. Catalyst was filtered off and filtrate was concentrated under reduced pressure to give an oil which was used for next reaction without purification. Phenyl dichlorophosphate (332 μL, 2.23 mmol) was dissolved in anhydrous dichloromethane (15 mL) and stirred under atmospheric nitrogen in an ice bath. The resulting product was dissolved in anhydrous dichloromethane (5 mL) and added dropwise. Triethylamine (684 μL, 4.9 mmol) was added dropwise and stirred for 1 hr. p-Nitrophenol (248 mg, 1.78 mmol) was added and ice bath was removed. Reaction was then stirred for 14 hrs. Reaction was diluted with dichloromethane (20 mL) and washed with water (3×20 mL). Organic layer was dried over anhydrous sodium sulfate and concentrated under reduced pressure. The crude product was purified via SiO₂ column chromatography (24 g SiO₂ Combiflash HP Gold Column 0-50% ethyl acetate/hexanes) to afford the product. ¹H NMR (400 MHz, Chloroform-d) δ 8.28-8.17 (m, 2H), 7.47-7.30 (m, 4H), 7.28-7.13 (m, 3H), 4.92 (m, 1H), 4.33-4.08 (m, 2H), 3.84 (m, 1H), 3.39 (m, 2H), 1.43 (s, 9H), 1.36 (d, J=7.2 Hz, 3H). ³¹P NMR (162 MHz, Chloroform-d) δ −1.07. MS m/z=532.0 [M+Na], 508.0 [M−1].

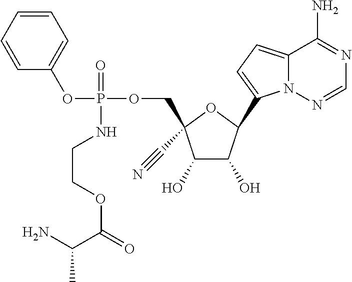 [SMILES: C[C@H](N)C(=O)OCCNP(=O)(OC[C@@]1(C#N)O[C@@H](c2ccc3c(N)ncnn23)[C@H](O)[C@@H]1O)Oc1ccccc1]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl L-alaninate. Intermediate 4 (50 mg, 0.15 mmol) and (2S)-2-(((4-nitrophenoxy)(phenoxy)phosphoryl)amino)ethyl 2-((tert-butoxycarbonyl) amino)propanoate (92 mg, 0.18 mmol) were dissolved in anhydrous tetrahydrofuran (3 mL). Magnesium chloride (21 mg, 0.225 mmol) was added in one portion. Reaction was warmed to 50° C. and stirred for 20 mins. N,N-Diisopropylethylamine (65 μL, 0.375 mmol) was added, and the reaction was stirred for 16 hrs at 50° C. Reaction was cooled to room temperature, diluted with ethyl acetate (30 mL) and washed with water (3×20 mL) and then brine (20 mL). Dried over anhydrous sodium sulfate and concentrated under reduced pressure. Residue was dissolved in acetonitrile (1.6 mL) and stirred in an ice bath. 12 M hydrochloric acid (160 μL) was added dropwise and stirred in an ice bath for 90 mins. Reaction was diluted with methanol (500 μL) and purified with prep HPLC (Phenomenex Gemini C₁₈ column, 5-95% acetonitrile/water with no acid modifier). Fractions having the desired product were combined and concentrated under reduced pressure. Residue was re-purified with prep HPLC (Phenomenex Gemini C₁₈ column, 5-70% acetonitrile/water with no acid modifier). Fractions having the desired product were combined and concentrated under reduced pressure. Residue was dissolved in acetonitrile and water and freeze dried to give the product. ¹H NMR (400 MHz, Methanol-d₄) δ 8.01 (m, 1H), 7.41-7.26 (m, 3H), 7.26-7.11 (m, 3H), 6.94 (m, 1H), 5.55 (m, 1H), 4.53 (m, 1H), 4.49-4.31 (m, 3H), 4.22 (m, 2H), 4.11-3.95 (m, 1H), 3.30-3.19 (m, 2H), 1.56-1.42 (m, 3H). ³¹P NMR (162 MHz, Methanol-d₄) δ 5.24, 5.18. MS m/z=562.1 [M+1], 559.9 [M−1].

### Example 134. (2S)-cycloheptyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

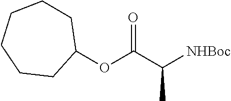 [SMILES: C[C@H](NC(=O)OC(C)(C)C)C(=O)OC1CCCCCC1]

(S)-cycloheptyl 2-((tert-butoxycarbonyl)amino)propanoate. Took up Cbz-L-alaninate (1.04 g, 5.49 mmol) in acetonitrile (10 mL) and added cycloheptanol (2.17 mL, 27.48 mmol) followed by EDCI (1.02 g, 6.59 mmol) and DMAP (1.01 g, 8.25 mmol) in one portion. Allowed to stir at room temperature overnight. Concentrated and diluted with CH₂Cl₂. Purified by silica gel chromatography 0-40% EtOAc/Hex to afford the intermediate. ¹H NMR (400 MHz, DMSO-d₆) δ 7.20 (d, J=7.3 Hz, 1H), 4.81 (tt, J=8.0, 4.2 Hz, 1H), 3.90 (p, J=7.4 Hz, 1H), 1.78 (dt, J=15.0, 10.6 Hz, 2H), 1.67-1.27 (m, 19H), 1.19 (d, J=7.3 Hz, 3H).

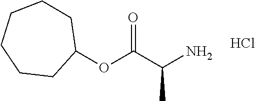

(S)-cycloheptyl 2-aminopropanoate hydrochloride. Took up (S)-cycloheptyl 2-((tert-butoxycarbonyl)amino)propanoate (1.00 g, 3.50 mmol) in CH₂Cl₂ (10 mL) and 4 N HCl in dioxane (10 mL, 40 mmol). Stirred at ambient temperature for 1 h. Concentrated under reduced pressure and co-evaporated with hexanes. Placed under high vacuum for 1 h and the intermediate was used as is without purification for the next step.

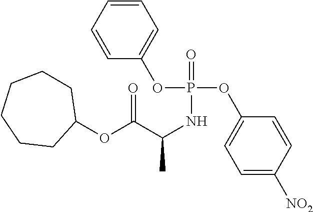 [SMILES: C[C@H](NP(=O)(Oc1ccccc1)Oc1ccc([N+](=O)[O-])cc1)C(=O)OC1CCCCCC1]

(2S)-cycloheptyl 2-(((4-nitrophenoxy)(phenoxy)phosphoryl)amino)propanoate. To a solution of (S)-cycloheptyl 2-aminopropanoate hydrochloride (0.77 g, 3.50 mmol) and phenyl dichlorophosphate (0.52 mL, 3.50 mmol) in dichloromethane (20 mL) was added triethylamine (1.0 mL, 7.02 mmol) at 0° C. under and argon atmosphere. The resulting mixture was allowed to warm to RT and was stirred for 1 h. 4-Nitrophenol (463 mg, 3.32 mmol) and triethylamine (0.53 mL, 3.51 mmol) were then added. After 2 h, the reaction mixture was diluted with Et₂O (100 mL) and the solids were filtered off. The crude product was concentrated under reduced pressure and was purified by silica gel chromatography (120 g SiO₂ Combiflash HP Gold Column, 0-50% ethyl acetate/hexanes) to afford the intermediate. ¹H NMR (400 MHz, DMSO-d₆) δ 8.40-8.16 (m, 2H), 7.60-7.32 (m, 4H), 7.32-7.13 (m, 3H), 6.64 (m, 1H), 4.75 (m, 1H), 3.92 (m, 1H), 1.83-1.65 (m, 2H), 1.65-1.42 (m, 8H), 1.34 (m, 2H), 1.26-1.07 (m, 3H). ³¹P NMR (162 MHz, DMSO-d₆) δ −1.22 (s), −1.46 (s). LC/MS: t_{R}=2.03 min, MS m/z=462.81 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min.

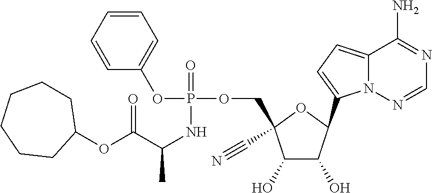 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCCC1]

(2S)-cycloheptyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. To a mixture of Intermediate 4 (50.0 mg, 0.12 mmol), intermediate (2S)-cycloheptyl 2-(((4-nitrophenoxy)(phenoxy)phosphoryl)amino)propanoate (64.3 mg, 0.140 mmol), and magnesium chloride (16.55 mg, 0.174 mmol) was added THF (1.0 mL) at RT. The resulting suspension was warmed to 50° C., and was allowed to stir for 10 min. N,N-Diisopropylethylamine (0.05 mL, 0.29 mmol) was then added and the resulting mixture was stirred at 50° C. for 1 h. Magnesium chloride (20 mg, 0.20 mmol) was added and stirred at 50° C. for 3 h. The reaction mixture was then allowed to cool to RT, and concentrated aqueous hydrochloric acid solution (12 M, 0.300 mL, 3.6 mmol) was added. After 1 h, the reaction mixture was cooled in an ice bath and quenched with saturated aqueous sodium carbonate solution to pH=7. The crude mixture was purified by preparatory HPLC (Phenominex Gemini NX 10 u C18 250×30 mm column, 40-100% acetonitrile/water gradient) to afford the product. LC/MS: t_{R}=1.39 min, MS m/z=615.02 [M+1]; LC system: Thermo Accela 1250 UHPLC. MS system: Thermo LCQ Fleet; Column: Kinetex 2.6μ XB-C18 100 A, 50×3.00 mm. Solvents: Acetonitrile with 0.1% formic acid, Water with 0.1% formic acid. Gradient: 0 min-2.4 min 2-100% ACN, 2.4 min-2.80 min 100% ACN, 2.8 min-2.85 min 100%-2% ACN, 2.85 min-3.0 min 2% ACN at 1.8 mL/min. ¹H NMR (400 MHz, DMSO-d₆) δ 7.84 (s, 1H), 7.74 (s, 2H), 7.44-7.25 (m, 3H), 7.25-7.08 (m, 2H), 6.84 (d, J=4.5 Hz, 1H), 6.71 (d, J=4.4 Hz, 1H), 6.20-5.90 (m, 2H), 5.48 (m, 1H), 5.38 (m, 1H), 4.75 (m, 1H), 4.45 (m, 1H), 4.28 (m, 2H), 4.16 (m, 1H), 1.74 (m, 3H), 1.51 (m, 7H), 1.31 (m, 2H), 1.23-1.03 (m, 3H). ³¹P NMR (162 MHz, DMSO-d₆) δ 3.32 (s), 3.29 (s).

Separation of the (S) and (R) Diastereomers. The product was purified via chiral preparatory HPLC (Chiralpak IC, 150×4.6 mm, Heptane 70% IPA 30%) to afford the diastereomers:

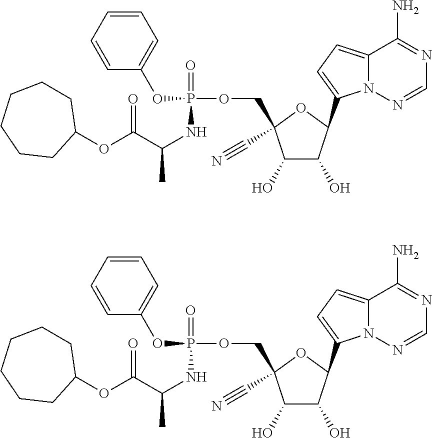 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCCCCC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCCCCC1]

### Example 135. First Eluting Diastereomer

¹H NMR (400 MHz, DMSO-d₆) δ 7.84 (s, 1H), 7.74 (br s, 2H), 7.34 (dd, J=8.6, 7.2 Hz, 2H), 7.25-7.07 (m, 3H), 6.84 (d, J=4.5 Hz, 1H), 6.71 (d, J=4.5 Hz, 1H), 6.17 (s, 1H), 6.06 (dd, J=13.3, 10.0 Hz, 1H), 5.53 (d, J=5.3 Hz, 1H), 5.37 (d, J=6.0 Hz, 1H), 4.73 (tt, J=8.3, 4.4 Hz, 1H), 4.45 (d, J=5.3 Hz, 1H), 4.13 (dd, J=10.9, 4.9 Hz, 1H), 3.76 (dtd, J=10.2, 7.1, 2.7 Hz, 1H), 1.84-1.62 (m, 2H), 1.62-1.38 (m, 8H), 1.38-1.23 (m, 2H), 1.17 (d, J=7.1 Hz, 3H), 1.02 (d, J=6.1 Hz, 2H). ³¹P NMR (162 MHz, DMSO-d₆) δ 3.32 (s).

### Example 136. Second Eluting Diastereomer

¹H NMR (400 MHz, DMSO-d₆) δ 7.83 (s, 1H), 7.74 (brs, 2H), 7.36-7.24 (m, 2H), 7.24-7.05 (m, 3H), 6.84 (d, J=4.5 Hz, 1H), 6.71 (d, J=4.5 Hz, 1H), 6.16-5.98 (m, 2H), 5.48 (d, J=5.9 Hz, 1H), 5.38 (d, J=6.1 Hz, 1H), 4.45 (q, J=5.8 Hz, 1H), 4.39-4.23 (m, 2H), 4.18 (dd, J=10.9, 4.9 Hz, 1H), 1.86-1.66 (m, 2H), 1.64-1.40 (m, 8H), 1.34 (s, 2H), 1.17 (d, J=7.2 Hz, 3H), 1.02 (d, J=6.1 Hz, 2H). ³¹P NMR (162 MHz, DMSO-d₆) δ 3.29 (s).

### Example 137. (2S)-cycloheptyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

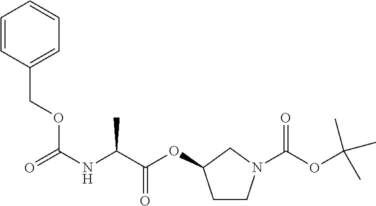 [SMILES: C[C@H](NC(=O)OCc1ccccc1)C(=O)O[C@@H]1CCN(C(=O)OC(C)(C)C)C1]

(R)-tert-butyl 3-(((S)-2-(((benzyloxy)carbonyl)amino)propanoyl)oxy)pyrrolidine-1-carboxylate. Cbz-L-Alanine (446 mg, 2 mmol) was dissolved anhydrous acetonitrile (15 mL). N-Ethyl-N′-(3-Dimethylaminopropyl)carbodiimide hydrochloride (422 mg, 2.2 mmol) was added and stirred for 15 mins. (R)-Boc-3-pyrrolidinol (374 mg, 2 mmol) was added in one portion. 4-(Dimethylamino)pyridine (269 mg, 2.2 mmol) were added and the reaction was stirred for 16 hrs. Reaction was diluted with ethyl acetate (20 mL) and washed with 5% aqueous citric acid solution (20 mL), saturated aqueous sodium bicarbonate solution (20 mL) and finally with brine (20 mL). Organic layer was dried over anhydrous sodium sulfate and concentrated under reduced pressure. The crude product was purified via SiO₂ column chromatography (24 g SiO₂ Combiflash HP Gold Column 0-40% ethyl acetate/hexanes) to afford the product. ¹H NMR (400 MHz, Chloroform-d) δ 7.41-7.28 (m, 5H), 5.28 (m, 2H), 5.11 (s, 2H), 4.45-4.26 (m, 1H), 3.66-3.25 (m, 4H), 2.04 (m, 2H), 1.47 (s, 9H), 1.41 (d, J=7.2 Hz, 3H).

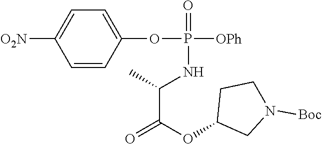 [SMILES: C[C@H](NP(=O)(Oc1ccccc1)Oc1ccc([N+](=O)[O-])cc1)C(=O)O[C@@H]1CCN(C(=O)OC(C)(C)C)C1]

(3R)-tert-butyl 3-(((2S)-2-(((4-nitrophenoxy)(phenoxy)phosphoryl)amino)propanoyl)oxy) pyrrolidine-1-carboxylate. (R)-tert-butyl 3-(((S)-2-(((benzyloxy)carbonyl)amino)propanoyl)oxy)pyrrolidine-1-carboxylate (693 mg, 1.76 mmol) was dissolved in tetrahydrofuran (40 mL). Degussa type 10% Palladium on carbon (100 mg) was added and the reaction was stirred under atmospheric hydrogen for 4 hrs. Catalyst was filtered off and filtrate was concentrated under reduced pressure and the resulting product was used for next reaction without purification. Phenyl dichlorophosphate (263 μL, 1.76 mmol) was dissolved in anhydrous dichloromethane (15 mL) and stirred under atmospheric nitrogen in an ice bath. Above prepared oil was dissolved in anhydrous dichloromethane (5 mL) and added dropwise. Triethylamine (544 μL, 4 mmol) was added dropwise and stirred for 1 hr. p-Nitrophenol (197 mg, 1.42 mmol) was added and ice bath was removed. Reaction was then stirred for 14 hrs. Reaction was diluted with dichloromethane (20 mL) and washed with water (3×20 mL). Organic layer was dried over anhydrous sodium sulfate and concentrated under reduced pressure. The crude product was purified via SiO₂ column chromatography (24 g SiO₂ Combiflash HP Gold Column 0-50% ethyl acetate/hexanes) to afford the product. ¹H NMR (400 MHz, Chloroform-d) δ 8.29-8.16 (m, 2H), 7.37 (m, 4H), 7.29-7.15 (m, 3H), 5.28 (s, 1H), 4.13 (m, 2H), 3.83 (t, J=10.6 Hz, 1H), 3.65-3.23 (m, 4H), 2.15-2.05 (m, 1H), 1.96 (m, 1H), 1.46 (s, 9H), 1.41 (d, J=7.1 Hz, 3H). ³¹P NMR (162 MHz, Chloroform-d) δ −3.21, −3.26. MS m/z=558.0 [M+Na], 534.1 [M−1].

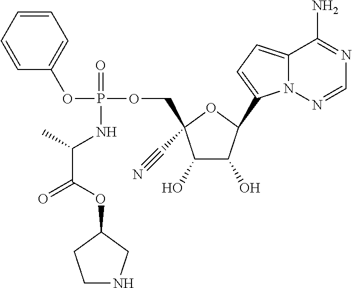 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@@H]1CCNC1]

(2S)—(R)-pyrrolidin-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. Intermediate 4 (73 mg, 0.22 mmol) and (3R)-tert-butyl 3-(((2S)-2-(((4-nitrophenoxy)(phenoxy)phosphoryl)amino)propanoyl)oxy) pyrrolidine-1-carboxylate (130 mg, 0.242 mmol) were dissolved in anhydrous acetonitrile (3 mL). Magnesium chloride (32 mg, 0.33 mmol) was added in one portion. Reaction was warmed to 50° C. and stirred for 30 mins. N,N-Diisopropylethylamine (65 μL, 0.375 mmol) was added, and the reaction was stirred for 16 hrs at 50° C. Reaction was cooled to room temperature, diluted with ethyl acetate (30 mL) and washed with 2% aqueous sodium carbonate solution (3×20 mL) and then brine (20 mL), dried over anhydrous sodium sulfate and concentrated under reduced pressure. Residue was dissolved in acetonitrile (1.6 mL) and stirred in an ice bath. 12 M hydrochloric acid (200 μL) was added dropwise and stirred in an ice bath for 9 hrs. Reaction was diluted with methanol (500 μL) and purified with prep HPLC (Phenomenex Gemini Cis column, 5-95% acetonitrile/water with no acid modifier). Fractions having the desired product were combined and freeze-dried. Residue was re-purified with prep HPLC (Phenomenex Gemini C₁₈ column, 5-70% acetonitrile/water with no acid modifier). Fractions having the desired product were combined and freeze-dried to give the product. ¹H NMR (400 MHz, Methanol-d₄) δ 7.84 (m, 1H), 7.41-7.10 (m, 5H), 6.96 (m, 1H), 6.78 (m, 1H), 5.50 (d, J=4.8 Hz, 1H), 5.43-5.27 (m, 1H), 4.63 (q, J=5.4 Hz, 1H), 4.56-4.29 (m, 3H), 3.94 (m, 1H), 3.52-3.32 (m, 4H), 2.34-2.06 (m, 2H), 1.30 (m, 3H). ³¹P NMR (162 MHz, Methanol-d₄) δ 3.41, 3.06. MS m/z=588.1 [M+1], 586.1 [M−1].

### Example 138. ((2R,3S,4R,5S)-5-(4-aminopyrrolo[2,1-f][1,2,4]triazin-7-yl)-2-cyano-3,4-dihydroxytetrahydrofuran-2-yl)methyl phenyl ((S)-1-(cyclohexylamino)-1-oxopropan-2-yl)phosphoramidate

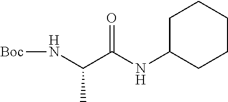 [SMILES: C[C@H](NC(=O)OC(C)(C)C)C(=O)NC1CCCCC1]

(S)-tert-butyl (1-(cyclohexylamino)-1-oxopropan-2-yl)carbamate. Boc-L-Alanine (378 mg, 2 mmol) was dissolved anhydrous tetrahydrofuran (10 mL). N-Ethyl-N′-(3-Dimethylaminopropyl)carbodiimide hydrochloride (422 mg, 2.2 mmol) was added and stirred for 20 mins. Cyclohexylamine (252 μL, 2.2 mmol) was added in one portion. Triethylamine (419 μL, 3 mmol) were added and the reaction was stirred for 20 hrs. Reaction was diluted with ethyl acetate (20 mL) and washed with 5% aqueous citric acid solution (2×20 mL), saturated aqueous sodium bicarbonate solution (2×20 mL) and brine (20 mL). Organic layer was dried over anhydrous sodium sulfate and concentrated under reduced pressure to afford the intermediate which was used for the next step without further purification. ¹H NMR (400 MHz, Methanol-d₄) δ 4.00 (m 1H), 3.62 (m, 1H), 1.94-1.68 (m, 4H), 1.61 (m, 1H), 1.43 (s, 9H), 1.40-1.29 (m, 2H), 1.26 (d, J=7.2 Hz, 3H), 1.21 (m, 3H).

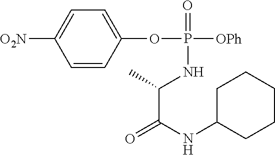 [SMILES: C[C@H](NP(=O)(Oc1ccccc1)Oc1ccc([N+](=O)[O-])cc1)C(=O)NC1CCCCC1]

4-nitrophenyl phenyl ((S)-1-(cyclohexylamino)-1-oxopropan-2-yl)phosphoramidate. (S)-tert-butyl (1-(cyclohexylamino)-1-oxopropan-2-yl)carbamate (232 mg, 0.859 mmol) was dissolved in 4 N hydrochloride in 1,4-dioxane (5 mL) and stirred for 30 mins. Reaction was concentrated under reduced pressure and the resulting product was used without purification. Above product was mixed with anhydrous dichloromethane (12 mL) and stirred under atmospheric nitrogen in an ice bath. Phenyl dichlorophosphate (128 μL, 0.859 mmol) was added in one portion. Triethylamine (264 μL, 1.89 mmol) was added dropwise and stirred for 1 hr. More triethylamine (132 μL, 0.945 mmol) was added dropwise and stirred for 30 mins. p-Nitrophenol (97 mg, 0.687 mmol) was added. Ice bath was removed and the reaction mixture was stirred for 14 hrs. Reaction was diluted with dichloromethane (20 mL) and washed with water (2×20 mL). Organic layer was dried over anhydrous sodium sulfate and concentrated under reduced pressure. The crude product was purified via SiO₂ column chromatography (4 g SiO₂ Combiflash HP Gold Column 0-50% ethyl acetate/hexanes) to afford the intermediate. MS m/z=448.1 [M+1], 446.1 [M−1].

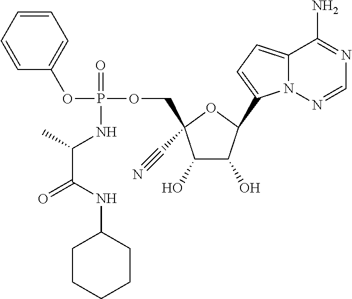 [SMILES: C[C@H](NP(=O)(OC[C@@]1(C#N)O[C@@H](c2ccc3c(N)ncnn23)[C@H](O)[C@@H]1O)Oc1ccccc1)C(=O)NC1CCCCC1]

((2R,3S,4R,5S)-5-(4-aminopyrrolo[2,1-f][1,2,4]triazin-7-yl)-2-cyano-3,4-dihydroxytetrahydrofuran-2-yl)methyl phenyl ((S)-1-(cyclohexylamino)-1-oxopropan-2-yl)phosphoramidate. Intermediate 4 (15 mg, 0.044 mmol) and 4-nitrophenyl phenyl ((S)-1-(cyclohexylamino)-1-oxopropan-2-yl)phosphoramidate (20 mg, 0.044 mmol) were dissolved in anhydrous tetrahydrofuran (3 mL). Magnesium chloride (6.5 mg, 0.068 mmol) was added in one portion. Reaction was warmed to 50° C. and stirred for 30 mins. N,N-Diisopropylethylamine (20 μL, 0.113 mmol) was added, and the reaction was stirred for 16 hrs at 50° C. Reaction was cooled to room temperature, diluted with ethyl acetate (30 mL) and washed with 5% aqueous sodium carbonate solution (3×20 mL) and then brine (20 mL). Dried over anhydrous sodium sulfate and concentrated under reduced pressure. Residue was dissolved in acetonitrile (1.6 mL) and stirred in an ice bath. 12 M hydrochloric acid (300 μL) was added dropwise and stirred in an ice bath for 2 hrs. Sodium bicarbonate (360 mg) was added in small portions. The crude product was purified with prep HPLC (Phenomenex Gemini C₁₈ column, 5-100% acetonitrile/water with no acid modifier). Fractions having the desired product were combined and freeze-dried to give the product. ¹H NMR (400 MHz, Methanol-d4) δ 7.99 (m, 1H), 7.52 (m, 1H), 7.42-7.09 (m, 5H), 6.95 (m, 1H), 5.54 (m, 1H), 4.53 (m, 1H), 4.47-4.29 (m, 3H), 3.78 (m, 1H), 3.62-3.43 (m, 1H), 1.84-1.51 (m, 5H), 1.26-1.00 (m, 8H). ³¹P NMR (162 MHz, Methanol-d₄) δ 3.68, 3.31. MS m/z=600.1 [M+1], 598.1 [M−1].

### Example 139. (2S)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 2-((tert-butoxycarbonyl)amino)-3-methylbutanoate

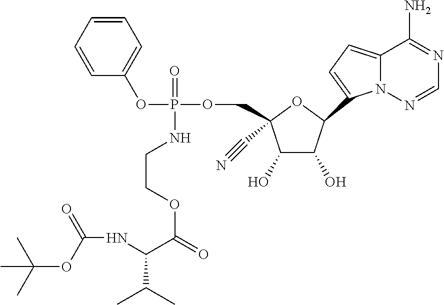 [SMILES: CC(C)[C@H](NC(=O)OC(C)(C)C)C(=O)OCCNP(=O)(OC[C@@]1(C#N)O[C@@H](c2ccc3c(N)ncnn23)[C@H](O)[C@@H]1O)Oc1ccccc1]

The product was isolated from the reaction for Example 131. ¹H NMR (400 MHz, Methanol-d₄) δ 7.80 (m, 1H), 7.41-7.06 (m, 5H), 6.87 (m, 1H), 6.74 (m, 1H), 5.51 (d, J=4.8 Hz, 1H), 4.69-4.57 (m, 1H), 4.57-4.28 (m, 3H), 4.14-3.91 (m, 3H), 3.26 (m, 1H), 3.16 (m, 2H), 2.18-1.96 (m, 1H), 1.43 (s, 9H), 0.88 (m, 6H). ³¹P NMR (162 MHz, Methanol-d₄) δ 5.15, 4.99. MS m/z=689.9 [M+1], 688.1 [M−1].

### Example 140. (3R)-tert-butyl 3-(((2R)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoyl)oxy)pyrrolidine-1-carboxylate

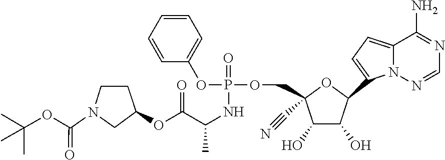 [SMILES: C[C@@H](NP(=O)(OC[C@@]1(C#N)O[C@@H](c2ccc3c(N)ncnn23)[C@H](O)[C@@H]1O)Oc1ccccc1)C(=O)O[C@@H]1CCN(C(=O)OC(C)(C)C)C1]

The product was also isolated from the reaction for Example 137. ¹H NMR (400 MHz, Methanol-d₄) δ 7.91 (m, 1H), 7.41-7.05 (m, 6H), 6.86 (m, 1H), 5.52 (m, 1H), 5.22 (m, 1H), 4.57 (m, 1H), 4.52-4.28 (m, 3H), 3.91 (m, 1H), 3.60-3.31 (m, 4H), 2.01 (m, 2H), 1.44 (s, 9H), 1.29 (m, 3H). ³¹P NMR (162 MHz, Methanol-d₄) δ 3.25, 3.22. MS m/z=688.0 [M+1], 686.1 [M−1].

### Example 141. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 1-methylcyclopropane-1-carboxylate

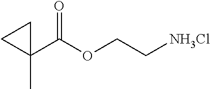

2-aminoethyl 1-methylcyclopropane-1-carboxylate hydrochloride. tert-Butyl (2-hydroxyethyl)carbamate (1.53 mL, 9.0 mmol) and 1-methylcyclopropane-1-carboxylic acid (0.92 mL, 9.0 mmol) were dissolved in acetonitrile (150 mL). EDCI (1.54 g, 10.0 mmol) and DMAP (1.21 g, 10.0 mmol) were then added and the reaction mixture was stirred at RT. After 20 h, the mixture was diluted with ethyl acetate (300 mL) and was washed with saturated aqueous sodium bicarbonate solution (300 mL) and brine (300 mL). The organic layer was dried over anhydrous sodium sulfate and was concentrated under reduced pressure. The crude oil was dissolved in dioxane (5 mL) and 4 M HCl in dioxane (20 mL) was added. After 2 h the resulting solids were collected by vacuum filtration to afford the intermediate. ¹H NMR (400 MHz, CDCl₃) δ 4.10 (t, J=5.3 Hz, 2H), 3.38 (q, J=5.5 Hz, 2H), 1.29 (s, 3H), 1.22 (q, J=3.9 Hz, 2H), 0.68 (q, J=3.9 Hz, 2H).

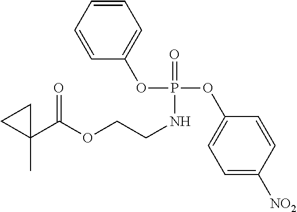 [SMILES: CC1(C(=O)OCCNP(=O)(Oc2ccccc2)Oc2ccc([N+](=O)[O-])cc2)CC1]

2-(((4-nitrophenoxy)(phenoxy)phosphoryl)amino)ethyl 1-methylcyclopropane-1-carboxylate. To a solution of intermediate 2-aminoethyl 1-methylcyclopropane-1-carboxylate hydrochloride (0.426 g, 2.37 mmol) and phenyl dichlorophosphate (0.500 mL, 2.37 mmol) in dichloromethane (11 mL) was added triethylamine (0.66 mL, 4.74 mmol) at 0° C. under and argon atmosphere. The resulting mixture was allowed to warm to RT and was stirred for 1 h. 4-Nitrophenol (330 mg, 2.37 mmol) and triethylamine (0.33 mL, 2.37 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, CDCl₃) δ 8.24 (dd, J=9.2, 2.1 Hz, 2H), 7.44-7.32 (m, 4H), 7.28-7.18 (m, 3H), 4.17-4.09 (m, 2H), 3.41-3.32 (m, 2H), 1.25 (d, J=1.6 Hz, 3H), 1.21-1.15 (m, 2H), 0.70-0.65 (m, 2H). ³¹P NMR (162 MHz, CD₃OD) δ −1.50 (s). MS m/z=421.04 [M+1].

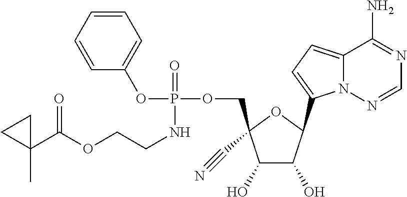 [SMILES: CC1(C(=O)OCCNP(=O)(OC[C@@]2(C#N)O[C@@H](c3ccc4c(N)ncnn34)[C@H](O)[C@@H]2O)Oc2ccccc2)CC1]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl 1-methylcyclopropane-1-carboxylate. To a mixture of Intermediate 4 (34.0 mg, 0.102 mmol), intermediate 2-(((4-nitrophenoxy)(phenoxy)phosphoryl)amino)ethyl 1-methylcyclopropane-1-carboxylate (43.0 mg, 0.102 mmol), and magnesium chloride (9.7 mg, 0.102 mmol) was added acetonitrile (0.50 mL) at RT. The resulting suspension was warmed to 50° C., and was allowed to stir for 5 min. N,N-Diisopropylethylamine (0.045 mL, 0.256 mmol) was then added and the resulting mixture was stirred at 50° C. for 1 h. The reaction mixture was then allowed to cool to RT, and concentrated aqueous hydrochloric acid solution (12 M, 0.119 mL) was added. After 1.5 h, the reaction mixture was diluted with saturated aqueous sodium carbonate solution (20 mL) and ethyl acetate (20 mL). The layers were split and the organic layer was washed with brine (20 mL), was dried over anhydrous sodium sulfate, and was concentrated under reduced pressure. The crude residue was purified via preparatory HPLC (Phenominex Luna 5 u C18(2) 100 Å 100×30 mm column, 5-100% acetonitrile/water gradient) to afford the product. ¹H NMR (400 MHz, methanol-d₄) δ 7.81-7.76 (m, 1H), 7.37-7.25 (m, 2H), 7.24-7.11 (m, 3H), 6.87-6.81 (m, 1H), 6.75-6.70 (m, 1H), 5.54-5.47 (m, 1H), 4.67-4.58 (m, 1H), 4.52-4.27 (m, 3H), 4.02-3.90 (m, 2H), 3.19-3.07 (m, 2H), 1.24-1.19 (m, 3H), 1.18-1.11 (m, 2H), 0.68-0.61 (m, 2H). ³¹P NMR (162 MHz, methanol-d₄) δ 5.24 (s), 5.06 (s). LCMS: MS m/z=573.31 [M+1], t_{R}=1.36 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.56 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=4.93 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 142. (2S)-(1r,4S)-4-methylcyclohexyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

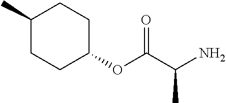

(S)-(1R,4S)-4-methylcyclohexyl 2-aminopropanoate. To a mixture of Cbz-L-alaninate (1.0 g, 4.48 mmol), trans-4-methylcyclohexanol (1.62 g, 14.21 mmol), and EDCI (0.83 g, 5.38 mmol) in acetonitrile (10 mL) was added DMAP (0.82 g, 6.72 mmol) in one portion. The resulting mixture was stirred at room temperature for 15 h, diluted with EtOAc, washed with brine, dried over sodium sulfate, and concentrated in vacuo. The obtained residue was purified by silica gel chromatography (EtOAc 0 to 50% in hexanes) to give a Cbz-L-alanine-trans-4-methylcyclohexyl ester, which was dissolved in THF (10 mL) and 20% palladium hydroxide on carbon (250 mg) was added. The resulting mixture was stirred under hydrogen gas balloon for 2 h, filtered through a celite pad. The filtrate was concentrated in vacuo, and dried under high vacuum to afford the intermediate which was used in next reaction. ¹H NMR (400 MHz, Chloroform-d) δ 4.66 (tt, J=11.1, 4.4 Hz, 1H), 3.49 (qd, J=7.0, 0.9 Hz, 1H), 1.93 (ddt, J=12.8, 6.0, 2.8 Hz, 2H), 1.83 (d, J=1.9 Hz, 2H), 1.77-1.66 (m, 2H), 1.43-1.22 (m, 6H), 1.10-0.93 (m, 2H), 0.88 (d, J=6.5 Hz, 3H). MS m/z=186 (M+H)⁺.

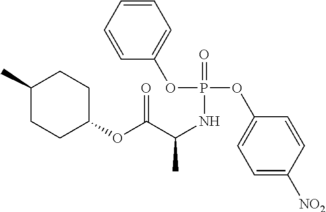 [SMILES: C[C@H](NP(=O)(Oc1ccccc1)Oc1ccc([N+](=O)[O-])cc1)C(=O)O[C@H]1CC[C@H](C)CC1]

(2S)-(1r,4S)-4-methylcyclohexyl 2-(((4-nitrophenoxy)(phenoxy)phosphoryl)amino)propanoate. (S)-(1R,4S)-4-methylcyclohexyl 2-aminopropanoate (470 mg, 2.54 mmol) was converted to this intermediate by the same procedure used for Intermediate 25. ¹H NMR (400 MHz, Chloroform-d) δ 8.22 (m, 2H), 7.37 (m, 4H), 7.28-7.13 (m, 3H), 4.65 (m, 1H), 4.18-4.01 (m, 1H), 3.88 (m, 1H), 1.96-1.81 (m, 2H), 1.80-1.60 (m, 2H), 1.45-1.22 (m, 6H), 1.11-0.95 (m, 2H), 0.89 (d, J=6.5 Hz, 3H). ³¹P NMR (162 MHz, Chloroform-d) δ −3.00, −3.06. MS m/z=463 (M+H)⁺.

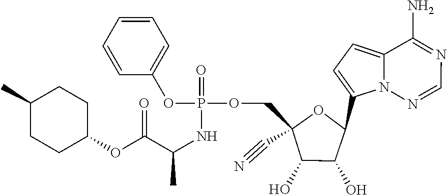 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1]

(2S)-(1r,4S)-4-methylcyclohexyl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. To a mixture of Intermediate 4 (50 mg, 0.15 mmol), intermediate (2S)-(1r,4S)-4-methylcyclohexyl 2-(((4-nitrophenoxy)(phenoxy)phosphoryl)amino)propanoate (116 mg, 0.23 mmol), and MgCl₂ (22 mg, 0.23 mmol) in THF (3 mL) was added N,N-diisopropylethylamine (0.066 mL, 0.38 mmol) dropwise at room temperature. The resulting mixture was stirred at 50° C. for 4 h, diluted with EtOAc, washed with water and brine, dried with sodium sulfate, and concentrated in vacuo. The resulting residue was dissolved in ACN (2 mL) and c-HCl (0.2 mL) was added. The mixture was stirred for 2 h and preparative HPLC (Phenominex Synergi 4 u Hydro-RR 80 Å 150×30 mm column, 10-70% acetonitrile/water gradient) to give the product. ¹H NMR (400 MHz, Methanol-d4) δ 7.79 (m, 1H), 7.37-7.08 (m, 5H), 6.84 (m, 1H), 6.73 (m, 1H), 5.50 (m, 1H), 4.70-4.47 (m, 2H), 4.47-4.29 (m, 3H), 3.85 (m, 1H), 1.92-1.76 (m, 2H), 1.73-1.61 (m, 2H), 1.38-1.20 (m, 6H), 1.05-0.82 (m, 5H). ³¹P NMR (162 MHz, Methanol-d₄) δ 3.30, 3.26. MS m/z=615 (M+H)⁺.

The product was separated by chiral preparatory HPLC (Chiralpak IA, 150×4.6 mm, Heptane 70%/Isopropanol 30%) to afford the diastereomers:

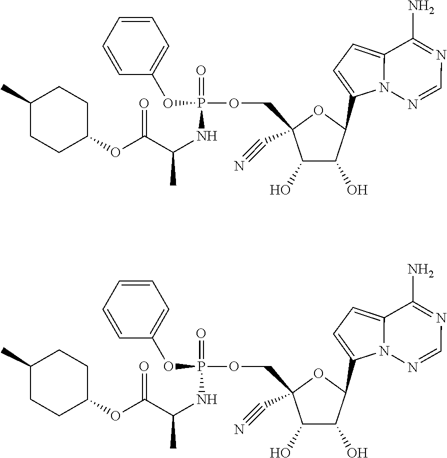 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1 ; C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C)CC1]

### Example 143. First Eluting Diastereomer

¹H NMR (400 MHz, Methanol-d4) δ 7.80 (s, 1H), 7.38-7.29 (m, 2H), 7.26-7.14 (m, 3H), 6.84 (d, J=4.6 Hz, 1H), 6.73 (d, J=4.5 Hz, 1H), 5.50 (d, J=5.0 Hz, 1H), 4.60 (t, J=5.3 Hz, 1H), 4.53 (tt, J=11.0, 4.3 Hz, 1H), 4.45 (d, J=5.6 Hz, 1H), 4.43-4.39 (m, 1H), 4.34 (dd, J=10.9, 5.6 Hz, 1H), 3.85 (dq, J=9.9, 7.1 Hz, 1H), 1.93-1.76 (m, 2H), 1.66 (ddt, J=13.5, 10.1, 3.2 Hz, 2H), 1.35-1.21 (m, 6H), 1.04-0.88 (m, 2H), 0.86 (d, J=6.5 Hz, 3H). ³¹P NMR (162 MHz, Methanol-d4) δ 3.26.

### Example 144. Second Eluting Diastereomer

¹H NMR (400 MHz, Methanol-d4) δ 7.78 (s, 1H), 7.34-7.21 (m, 2H), 7.22-7.09 (m, 3H), 6.85 (d, J=4.5 Hz, 1H), 6.73 (d, J=4.6 Hz, 1H), 5.51 (d, J=5.0 Hz, 1H), 4.65-4.53 (m, 2H), 4.51 (d, J=5.6 Hz, 1H), 4.47 (dd, J=10.9, 6.0 Hz, 1H), 4.35 (dd, J=10.9, 5.2 Hz, 1H), 3.92-3.75 (m, 1H), 1.87 (d, J=12.1 Hz, 2H), 1.70 (d, J=13.2 Hz, 2H), 1.40-1.26 (m, 3H), 1.24 (dd, J=7.1, 1.2 Hz, 3H), 1.00 (q, J=12.8 Hz, 2H), 0.88 (d, J=6.5 Hz, 3H). ³¹P NMR (162 MHz, Methanol-d4) δ 3.30.

### Example 145. (2S)-piperidin-4-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

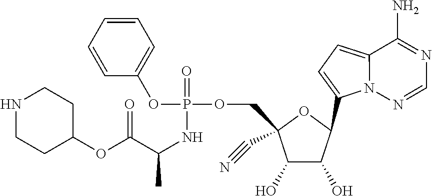 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCNCC1]

To a mixture of Intermediate 4 (52 mg, 0.16 mmol), Intermediate 26 (144 mg, 0.24 mmol), and MgCl₂ (23 mg, 0.24 mmol) in THF (3 mL) was added N,N-diisopropylethylamine (0.069 mL, 0.43 mmol) dropwise at room temperature. The resulting mixture was stirred at 50° C. for 2 h, diluted with EtOAc, washed with water and brine, dried with sodium sulfate, and concentrated in vacuo. The resulting residue was dissolved in ACN (2 mL) and c-HCl (0.2 mL) was added. The mixture was stirred at room temperature for 2 h and purified by preparative HPLC (Phenominex Gemini 10 u 250×21 mm column, 0-60% 1% TFA acetonitrile/water gradient) to afford the product as a trifluoroacetic acid salt. ¹H NMR (400 MHz, Methanol-d4) δ 8.02 (m, 1H), 7.47-7.11 (m, 6H), 6.95 (m, 1H), 5.54 (m, 1H), 5.01 (m, 1H), 4.54 (m, 1H), 4.50-4.34 (m, 3H), 3.99 (m, 1H), 3.28 (m, 2H), 3.17 (m, 2H), 2.02 (m, 2H), 1.97-1.86 (m, 2H), 1.35 (dt, J=7.2, 1.8 Hz, 3H). ³¹P NMR (162 MHz, Methanol-d4) δ 3.32, 3.17. ¹⁹F NMR (376 MHz, Methanol-d4) δ −77.95. MS m/z=602 (M+H)⁺.

### Example 146. (2S)—(R)-1-acetylpyrrolidin-3-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

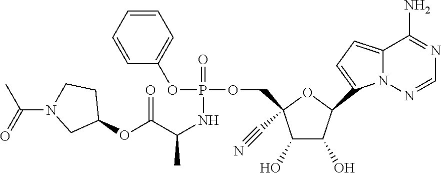 [SMILES: CC(=O)N1CC[C@@H](OC(=O)[C@H](C)NP(=O)(OC[C@@]2(C#N)O[C@@H](c3ccc4c(N)ncnn34)[C@H](O)[C@@H]2O)Oc2ccccc2)C1]

Acetic acid (4.8 μL, 0.084 mmol) was dissolved in anhydrous dichloromethane (300 μL). N-Ethyl-N′-(3-Dimethylaminopropyl)carbodiimide hydrochloride (16 mg, 0.084 mmol) was added in one portion and stirred for 30 mins. Triethylamine (12 μL, 0.084 mmol) was added and stirred for 30 mins. Example 137 (15 mg, 0.0255 mmol) was dissolved in anhydrous N,N-dimethylformamide (300 μL) and pyridine (150 μL). Above prepared mixture was added in 2 portions to the reaction which was then stirred for 14 hrs. Reaction was diluted with ethyl acetate (20 mL) and washed with saturated aqueous sodium bicarbonate solution (20 mL) and brine (20 mL). Organic layer was dried over anhydrous sodium sulfate and concentrated under reduced pressure. The crude product was purified via SiO₂ column chromatography (4 g SiO₂ Combiflash HP Gold Column 0-20% methanol/dichloromethane). Fractions having the desired product were combined and concentrated under reduced pressure. Residue was dissolved in acetonitrile and water and freeze-dried to give the product. ¹H NMR (400 MHz, Methanol-d₄) δ 7.79 (m, 1H), 7.41-7.07 (m, 5H), 6.89-6.79 (m, 1H), 6.74 (m, 1H), 5.55-5.48 (m, 1H), 5.35-5.15 (m, 1H), 4.62 (m, 1H), 4.58-4.28 (m, 3H), 3.90 (m, 1H), 3.69-3.44 (m, 4H), 2.09 (m, 2H), 1.96 (m, 3H), 1.26 (m, 3H). ³¹P NMR (162 MHz, Methanol-d₄) δ 3.14, 3.11. MS m/z=630.4 [M+1], 628.6 [M−1].

### Example 147. ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl (2-chlorophenyl) (2-(octadecyloxy)ethyl) phosphate

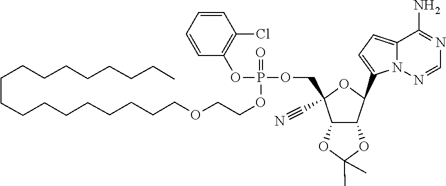 [SMILES: CCCCCCCCCCCCCCCCCCOCCOP(=O)(OC[C@@]1(C#N)O[C@@H](c2ccc3c(N)ncnn23)[C@@H]2OC(C)(C)O[C@@H]21)Oc1ccccc1Cl]

To a solution of 1,2,4-triazole (27.83 mg, 0.4 mmol), and triethylamine (0.06 mL, 0.4 mmol) in THF (0.50 mL) was added 2-chlorophenylphosphorodichloridate (0.03 mL, 0.2 mmol) at RT. After 1 h, the reaction mixture was filtered to remove the solids. Intermediate 4 (50 mg, 0.15 mmol) and 1-methylimidazole (0.02 mL, 0.2 mmol) were then added. After 1.5 h, ethylene glycol monooctadecyl ether (47.47 mg, 0.15 mmol) was added. After 20 h, the reaction mixture was diluted with ethyl acetate (10 mL) and was washed with water (10 mL). The organic layer was split and was dried over anhydrous sodium sulfate and was concentrated under reduced pressure. The crude residue was purified via SiO₂ column chromatography (4 g SiO₂ Combiflash HP Gold Column, 0-100% ethyl acetate/hexanes) to afford the product. ³¹P NMR (162 MHz, DMSO-d₆) δ −7.76 (s). MS m/z=818.34 [M+1].

### Example 148. ((2R,3S,4R,5S)-5-(4-aminopyrrolo[2,1-f][1,2,4]triazin-7-yl)-2-cyano-3,4-dihydroxytetrahydrofuran-2-yl)methyl (2-(octadecyloxy)ethyl) hydrogen phosphate

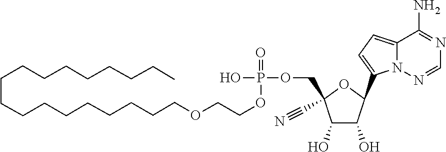 [SMILES: CCCCCCCCCCCCCCCCCCOCCOP(=O)(O)OC[C@@]1(C#N)O[C@@H](c2ccc3c(N)ncnn23)[C@H](O)[C@@H]1O]

Tetrabutylammonium fluoride (1 M in THF, 0.22 mL, 0.22 mmol) was added to a solution of Example 147 (57.0 mg, 0.07 mmol) in Pyridine (0.3 mL), Water (0.3 mL), and THF (2 mL) at RT. After 4 h, the reaction mixture was diluted with dichloromethane (2 mL) and water (2 mL). The aqueous layer was acidified to pH=3 with 1 N aqueous hydrochloric acid solution. The phases were split and the aqueous layer was extracted with dichloromethane (2×2 mL). The combined organic layers were dried over anhydrous sodium sulfate, and were concentrated under reduced pressure. The crude residue was dissolved in THF (2 mL), and concentrated hydrochloric acid solution (12 M, 100 μL) was added After 6 h, was basified to pH=4 with 2 N NaOH. The resulting mixture was concentrated under reduced pressure. The crude residue was purified via SiO₂ column chromatography (4 g SiO₂ Combiflash HP Gold Column, 0-20% methanol/dichloromethane) to afford the product. ¹H NMR (400 MHz, CD₃OD) δ 7.81 (s, 1H), 6.88 (d, J=4.5 Hz, 1H), 6.82 (d, J=4.5 Hz, 1H), 5.53 (d, J=5.3 Hz, 1H), 4.58 (t, J=5.4 Hz, 1H), 4.51 (d, J=5.6 Hz, 1H), 4.14 (qd, J=10.8, 4.8 Hz, 2H), 3.94 (q, J=5.5 Hz, 2H), 3.50 (t, J=5.1 Hz, 2H), 3.38 (t, J=6.7 Hz, 2H), 3.19 (q, J=7.3 Hz, 1H), 1.35-1.19 (m, 32H), 0.89 (t, J=6.7 Hz, 3H). ³¹P NMR (162 MHz, CD₃OD) δ −0.58 (s). MS m/z=668.20 [M+H]

### Example 149. ((2R,3S,4R,5S)-5-(4-aminopyrrolo[2,1-f][1,2,4]triazin-7-yl)-2-cyano-3,4-dihydroxytetrahydrofuran-2-yl)methyl (3-(hexadecyloxy)propyl) hydrogen phosphate

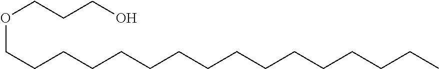 [SMILES: CCCCCCCCCCCCCCCCOCCCO]

3-(hexadecyloxy)propan-1-ol. Sodium hydride 60% dispersion in mineral oil (840 mg, 21 mmol) was mixed with anhydrous tetrahydrofuran (20 mL) and stirred under atmospheric nitrogen in an ice bath. 1,3-Propanediol (1.44 mL, 20 mmol) was added dropwise and stirred for 30 mins. 1-Bromo hexadecane (6.11 mL, 20 mmol) was added in one portion. Ice bath was removed and stirred for 1 hr. Reaction was heated to 80° C. and stirred for 4 hrs. Anhydrous N,N-dimethylformamide (10 mL) was added and stirred at 80° C. for 14 hrs. Reaction was cooled to room temperature, diluted with ethyl acetate (40 mL) and washed with 10% aqueous sodium carbonate solution (20 mL) and then brine (20 mL). Dried over anhydrous sodium sulfate and concentrated under reduced pressure. The crude product was purified via SiO₂ column chromatography (24 g SiO₂ Combiflash HP Gold Column 0-20% ethyl acetate/hexanes) to afford the intermediate. ¹H NMR (400 MHz, Chloroform-d) δ 3.78 (t, J=5.5 Hz, 2H), 3.61 (t, J=5.7 Hz, 2H), 3.42 (t, J=6.6 Hz, 2H), 1.83 (p, J=5.6 Hz, 2H), 1.56 (p, J=6.8 Hz, 2H), 1.25 (s, 26H), 0.88 (t, J=6.7 Hz, 3H).

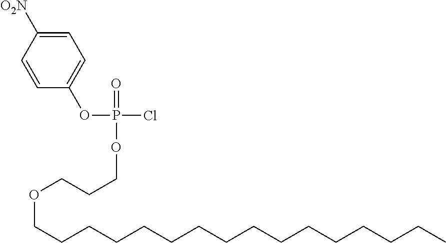 [SMILES: CCCCCCCCCCCCCCCCOCCCOP(=O)(Cl)Oc1ccc([N+](=O)[O-])cc1]

3-(hexadecyloxy)propyl bis(4-nitrophenyl) phosphate. p-Nitrophenyl dichlorophosphate (256 mg, 1 mmol) was dissolved in anhydrous dichloromethane (5 mL) and stirred under atmospheric nitrogen in an ice bath. 3-(hexadecyloxy)propan-1-ol (300 mg, 1 mmol) was dissolved in anhydrous dichloromethane (2 mL) and added to the reaction dropwise. Triethylamine (153 μL, 1.1 mmol) was added dropwise and stirred for 1 hr. Ice bath was removed and stirred for 4 hrs. p-Nitrophenol (111 mg, 0.8 mmol) was added along with triethylamine (153 μL, 1.1 mmol). Reaction mixture was stirred for 14 hrs. Reaction was diluted with ethyl acetate (20 mL) and washed with water (2×20 mL), dried over anhydrous sodium sulfate and concentrated under reduced pressure. The crude product was purified via SiO₂ column chromatography (12 g SiO₂ Combiflash HP Gold Column 0-20% ethyl acetate/hexanes) to afford the intermediate. ¹H NMR (400 MHz, Chloroform-d) δ 8.32-8.18 (m, 2H), 7.39 (m, 2H), 4.45 (q, J=6.6 Hz, 1H), 4.29 (q, J=6.6 Hz, 1H), 3.48 (dd, J=6.7, 5.4 Hz, 2H), 3.36 (td, J=6.7, 3.0 Hz, 2H), 1.98 (dt, J=16.4, 6.2 Hz, 2H), 1.53 (m, 2H), 1.25 (m, 26H), 0.87 (t, J=6.6 Hz, 3H). ³¹P NMR (162 MHz, Chloroform-d) δ −13.35.

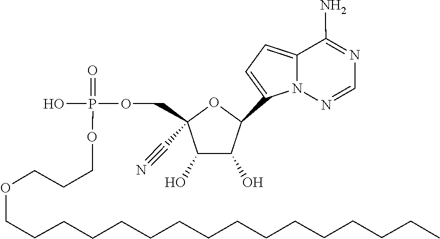 [SMILES: CCCCCCCCCCCCCCCCOCCCOP(=O)(O)OC[C@@]1(C#N)O[C@@H](c2ccc3c(N)ncnn23)[C@H](O)[C@@H]1O]

((2R,3S,4R,5S)-5-(4-aminopyrrolo[2,1-f][1,2,4]triazin-7-yl)-2-cyano-3,4-dihydroxytetrahydrofuran-2-yl)methyl (3-(hexadecyloxy)propyl) hydrogen phosphate. Intermediate 4 (50 mg, 0.15 mmol) and 3-(hexadecyloxy)propyl bis(4-nitrophenyl) phosphate (112 mg, 0.18 mmol) were dissolved in anhydrous tetrahydrofuran (3 mL). Magnesium chloride (71 mg, 0.75 mmol) was added in one portion. Reaction was warmed to 50° C. and stirred for 15 mins. N,N-Diisopropylethylamine (130 μL, 0.75 mmol) was added, and the reaction was stirred for 2 hrs at 50° C. Reaction was cooled to room temperature and diluted with ethyl acetate (50 mL). The mixture was filtered, and the filtrate was concentrated under reduced pressure. The crude product was purified via SiO₂ column chromatography (4 g SiO₂ Combiflash HP Gold Column 0-100% ethyl acetate/hexanes). Fractions having the desired product were combined and concentrated under reduced pressure. The material was dissolved in 1,4-dioxane (2 mL) and stirred in an ice bath. 0.1 N aqueous sodium hydroxide solution (1 mL) was added and the reaction was stirred for 3 hrs. The reaction was neutralized with 1 N hydrochloric acid to give pH of 7 and then extracted with ethyl acetate (30 mL). Organic layer was dried over anhydrous sodium sulfate and concentrated under reduced pressure. The crude product was purified via SiO₂ column chromatography (4 g SiO₂ Combiflash HP Gold Column 0-20% methanol/dichloromethane). Fractions having the desired product were combined and concentrated under reduced pressure to give white solid (29 mg). Material was dissolved in acetonitrile (2 mL). 12 N hydrochloric acid (300 uL) was added dropwise and then stirred for 1 hr. Reaction was concentrated under reduced pressure to afford the product. ¹H NMR (400 MHz, Methanol-d₄) δ 8.08 (s, 1H), 7.46 (d, J=4.5 Hz, 1H), 7.02 (d, J=4.5 Hz, 1H), 5.56 (d, J=4.7 Hz, 1H), 4.52 (t, J=5.1 Hz, 1H), 4.40 (d, J=5.1 Hz, 1H), 4.29 (qd, J=11.0, 5.5 Hz, 2H), 4.11 (q, J=6.5 Hz, 2H), 3.49 (t, J=6.0 Hz, 2H), 3.39 (t, J=6.5 Hz, 2H), 1.89 (p, J=6.2 Hz, 2H), 1.52 (q, J=6.7 Hz, 2H), 1.27 (s, 26H), 0.89 (t, J=6.7 Hz, 3H). ³¹P NMR (162 MHz, Methanol-d₄) δ −1.56.

### Example 150. (2S)-piperidin-4-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate

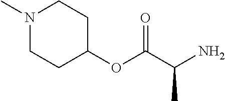

(S)-1-methylpiperidin-4-yl 2-aminopropanoate. Cbz-L-alaninate (2.33 g, 10.42 mmol), N-methyl-4-hydroxy-piperidine (1.00 g, 7.99 mmol), and HATU (3.47 g, 9.12 mmol) were dissolved in DMF (10 mL) and the resulting mixture was stirred at room temperature for 15 min. Then triethylamine (2.41 mL, 17.37 mmol) was added at once. The resulting mixture was stirred at room temperature for 15 h, diluted with EtOAc, washed with brine, dried over sodium sulfate, and concentrated in vacuo. The obtained residue was purified by silica gel chromatography (MeOH 0 to 20% in methylene chloride), which was dissolved in THF (15 mL) and 20% palladium hydroxide on carbon (250 mg) was added. The resulting mixture was stirred at room temperature for 4 h, filtered, concentrated in vacuo, co-evaporated with toluene several times, and dried under high vacuum for 15 h to afford the intermediate. ¹H NMR (400 MHz, Methanol-d4) δ 5.01-4.87 (m, 1H), 3.83 (q, J=7.2 Hz, 1H), 2.92 (m, 2H), 2.71 (m, 2H), 2.50 (s, 3H), 2.01 (m, 2H), 1.94-1.76 (m, 2H), 1.43 (d, J=7.1 Hz, 3H).

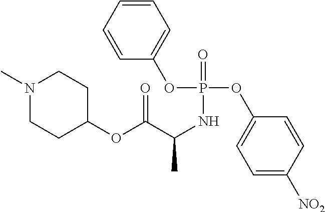 [SMILES: C[C@H](NP(=O)(Oc1ccccc1)Oc1ccc([N+](=O)[O-])cc1)C(=O)OC1CCN(C)CC1]

(2S)-1-methylpiperidin-4-yl 2-(((4-nitrophenoxy)(phenoxy)phosphoryl)amino)propanoate. (S)-1-methylpiperidin-4-yl 2-aminopropanoate (400 mg, 2.15 mmol) was dissolved in methylene chloride (5 mL), cooled to −78° C., and phenyl dichlorophosphate (0.32 mL, 2.15 mmol) added quickly. Triethylamine (0.30 mL, 2.15 mmol) was added over 30 min at −78° C. and 4-nitrophenol (299 mg, 2.15 mmol) was added in one portion. Then triethylamine (0.30 mL, 2.15 mmol) was added over 30 min at −78° C. The mixture was stirred for 2 h at −78° C. and 15 h at room temperature, diluted with methylene chloride, washed with water twice and brine, dried over sodium sulfate, and concentrated in vacuo. The residue was purified by silica gel column chromatography (MeOH 0 to 10% in methylene chloride) to give the intermediate. ¹H NMR (400 MHz, Chloroform-d) δ 8.21-8.00 (m, 2H), 7.50-7.04 (m, 7H), 4.90 (m, 1H), 4.08 (m, 1H), 2.96 (m, 4H), 2.55 (m, 3H), 2.08 (m, 2H), 1.87 (m, 2H), 1.35 (m, 3H). ³¹P NMR (162 MHz, Chloroform-d) δ −2.48, −2.79. MS m/z−464 (M+H)⁺.

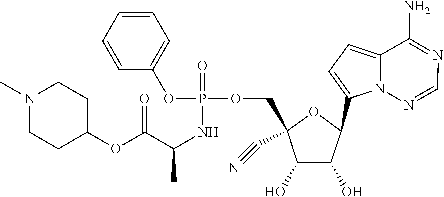 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCN(C)CC1]

(2S)-piperidin-4-yl 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)propanoate. To a mixture of Intermediate 4 (52 mg, 0.16 mmol), (2S)-1-methylpiperidin-4-yl 2-(((4-nitrophenoxy)(phenoxy)phosphoryl)amino)propanoate (135 mg, 0.246 mmol), and MgCl₂ (23 mg, 0.24 mmol) in THF (3 mL) was added N,N-diisopropylethylamine (0.069 mL, 0.43 mmol) dropwise at room temperature. The resulting mixture was stirred at 50° C. for 15 h, purified by preparative HPLC (Phenominex Synergi 4 u Hydro-RR 80 Å 150×30 mm column, 10-100% acetonitrile/water gradient) to give an acetonide intermediate (60 mg, 58%) as a white solid. 20 mg of the acetonide was dissolved in ACN (1 mL) and c-HCl (0.1 mL) added. The resulting mixture stirred at room temperature for 1 h and purified by preparative HPLC (Phenominex Gemini 10 u 250×21 mm column, 0-60% 1% TFA acetonitrile/water gradient) to give the product as TFA salt. ¹H NMR (400 MHz, Methanol-d4) δ 7.99 (m, 1H), 7.46-7.08 (m, 6H), 6.92 m, 1H), 5.53 (d, J=5.1 Hz, 1H), 5.06 (m, 1H), 4.56 (m, 1H), 4.52-4.31 (m, 3H), 4.12-3.89 (m, 1H), 3.55 (m, 1H), 3.34 (s, 1H), 3.24-3.09 (m, 2H), 2.84 (m, 3H), 2.23 (m 1H), 2.04 (m, 2H), 1.83 (s, 1H), 1.42-1.24 (m, 3H). ³¹P NMR (162 MHz, Methanol-d4) δ 3.36, 3.18. ¹⁹F NMR (376 MHz, Methanol-d4) δ −77.67. MS m/z=616 (M+H)⁺.

### Example 151. 3-(((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(hydroxy)phosphoryl)oxy)propane-1,2-diyl dipalmitate

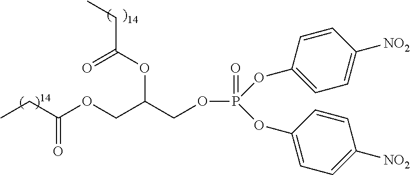

3-((bis(4-nitrophenoxy)phosphoryl)oxy)propane-1,2-diyl-dipalmitate. To a solution of 1,2-dipalmitoyl-rac-glycerol (562 mg, 0.988 mmol) and POCl₃ (167 mg, 1.087 mmol) in CH₂Cl₂ (5 mL) at −30° C. was added TEA (0.14 mL, 1.0 mmol). The reaction mixture was stirred at −30° C. for 10 minutes, then slowly warmed up to RT and stirred for 0.5 h. A solution of 4-nitrophenol (261 mg, 1.877 mmol) and TEA (0.286 mL, 2.06 mmol) in CH₂Cl₂ (1.5 mL) was dropwise added at 0° C. The resulted reaction mixture was stirred at 0° C. for 0.5 h, then at RT for 0.5 h. The reaction mixture was diluted with hexane (15 mL), and filtered. The filtrate was loaded on to a silica gel column (˜25 mL), eluted with 20-33% CH₃CO₂Et-hexane, and the fractions containing product were combined and concentrated under reduced pressure to afford the intermediate. ¹H NMR (400 MHz, CDCl₃): δ 8.28 (d, J=8.8 Hz, 4H), 7.40 (2d, J=9.2 Hz, 4H), 5.25-5.35 (m, 1H), 4.38-4.52 (m, 2H), 4.31 (dd, J=12, 4.4 Hz, 1H), 4.17 (d, J=12, 5.2 Hz, 1H), 2.20-2.32 (m, 4H), 1.50-1.70 (m, 8H), 1.25 (brs, 44H), 0.88 (t, J=7.2 Hz, 6H). ³¹P NMR (162 MHz, CDCl₃): δ −13.24.

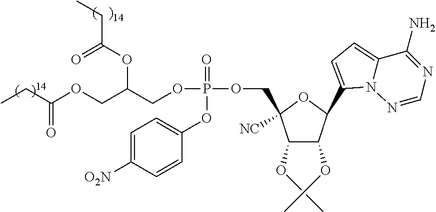

3-(((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(4-nitrophenoxy)phosphoryl)oxy)propane-1,2-diyl dipalmitate. To a solution of the Intermediate 4 (124 mg, 0.374 mmol) and the phosphate 3-((bis(4-nitrophenoxy)phosphoryl)oxy)propane-1,2-diyl-dipalmitate (335 mg, 0.376 mmol) in THF was added MgCl₂ (60 mg, 0.63 mmol). The reaction mixture was stirred at RT for 10 minutes, then N,N-Diisopropylethylamine (150 mg, 1.16 mmol) was added. The reaction mixture was stirred at RT for 16 h. TLC showed the reaction was completed. The reaction mixture was diluted with hexane-CH₃CO₂Et (1:1, 5 mL), washed with H₂O, dried over MgSO₄, purified by silica gel column (eluted with 30-70% CH₃CO₂Et-hexane) to give the desired product. ¹H NMR (400 MHz, CDCl₃): δ 8.10 (d, J=8.8 Hz, 1H), 7.97 (d, J=8.4 Hz, 1H), 7.89 (d, J=4 Hz, 1H), 7.30 (d, J=7.6 Hz, 1H), 7.21 (d, J=8.4, 1H), 6.68-6.75 (m, 2H), 5.61 (d, J=18.4 Hz, 1H), 5.15-5.27 (m, 2H), 5.03-5.05 (m, 1H), 4.4-4.58 (m, 2H), 4.2-4.4 (m, 2H), 4.05-4.18 (m, 2H), 2.2-2.3 (m, 4H), 2.1 (brs, 4H), 1.76 (d, J=32. Hz, 3H), 1.58 (brs, 4H), 1.36 (d, J=6.4 Hz, 3H), 1.25 (brs, 44H), 0.87 (t, J=6.4 Hz, 6H). ³¹P NMR (162 MHz, CDCl₃): δ −7.47, −8.21.

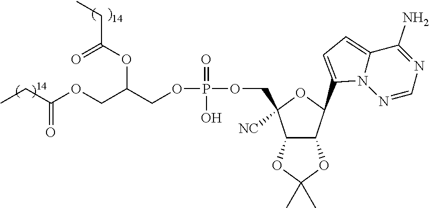

To a solution of 3-(((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(4-nitrophenoxy)phosphoryl)oxy)propane-1,2-diyl dipalmitate (122 mg, 0.113 mmol) in CH₃CO₂Et (2 mL) and CH₃CN (2 mL) was added H₂O (0.5 mL) and triethylamine (0.5 mL). The reaction mixture was stirred at RT for 36 h. The reaction mixture was concentrated. The residues was dissolved in CH₃CO₂Et (5 mL), AcOH (20 μL) was added, stirred for 10 minutes, then washed with H₂O and dried over MgSO₄. The crude material was purified by silica gel column, eluted with 5-25% MeOH—CH₂Cl₂ to afford the desired product. ¹H NMR (400 MHz, DMSO-d6+20% of CDCl₃): δ 7.9 (brs, 2H), 7.84 (s, 1H), 6.88 (d, J=4.4 Hz, 1H), 6.75 (d, J=4.4 Hz, 1H), 5.56 (d, J=3.6 Hz, 1H), 5.2-5.24 (m, 1H), 5.05-5.1 (m, 1H), 5.02 (d, J=6.8 Hz, 1H), 4.24 (dd, J=12, 2.8 Hz, 1H), 3.9-4.1 (m, 3H), 3.8-3.9 (m, 2H), 2.16-2.22 (m, 4H), 1.62 (s, 3H), 1.4-1.5 (m, 4H), 1.32 (s, 3H), 1.19 (brs, 48H), 0.82 (t, J=7 Hz, 6H). ³¹P NMR (162 MHz, DMSO-d6+20% of CDCl₃): δ −1.99.

### Example 152. 3-(((((2R,3S,4R,5S)-5-(4-aminopyrrolo[2,1-f][1,2,4]triazin-7-yl)-2-cyano-3,4-dihydroxytetrahydrofuran-2-yl)methoxy)(hydroxy)phosphoryl)oxy)propane-1,2-diyl dipalmitate

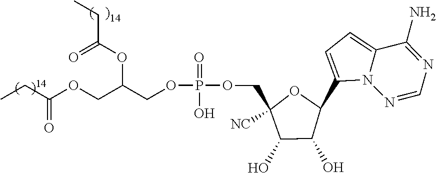

To a solution of Example 151 (40 mg, 0.042 mmol) in THF (1 mL) and H₂O (0.2 mL) was added HCl (37%, 0.2 mL). The reaction mixture was stirred at RT for 1 h. The reaction mixture was concentrated, and co-evaporated with THF-H₂O (2:1) twice. The residue was dissolved in CH₂Cl₂ and loaded to a silica gel column, eluted with 10-40% MeOH—CH₂Cl₂. The fractions containing product were combined and concentrated under reduced pressure. The residue was dissolved in CH₂Cl₂ and filtered. The filtrate was concentrated and treated with MeCN—H₂O (1:1), filtered, dried to give the product. ¹H NMR (400 MHz, DMSO-d6+20% of CDCl₃): δ 8.2 (brs, 2H), 7.91 (s, 1H), 6.97 (d, J=4 Hz, 1H), 6.76 (d, J=4 Hz, 1H), 5.37 (d, J=5.6 Hz, 1H), 5.05-5.15 (m, 1H), 4.41 (d, J=5.2 Hz, 1H), 4.2-4.3 (m, 2H), 3.8-4.3 (m, 5H), 2.15-2.3 (m, 4H), 1.4-1.5 (m, 4H), 1.19 (brs, 48H), 0.83 (t, J=6.4 Hz, 6H). ³¹P NMR (400 MHz, CDCl₃): δ −2.4.

### Example 153. (S)-2-oxopyrrolidin-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

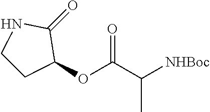 [SMILES: CC(NC(=O)OC(C)(C)C)C(=O)O[C@H]1CCNC1=O]

(S)-2-oxopyrrolidin-3-yl (tert-butoxycarbonyl)alaninate. The intermediate was prepared in a manner similar to that described for Example 117. ¹H NMR (400 MHz, Chloroform-d) δ 7.57 (s, 0.26H), 7.48 (s, 0.74H), 5.33-5.20 (m, 1H), 5.15 (brs, 1H), 4.38-4.22 (m, 1H), 3.39 (m, 1H), 3.30 (m, 1H), 2.61-2.46 (m, 1H), 2.12-1.97 (m, 1H), 1.37 (m, 12H).

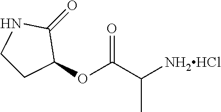

(S)-2-oxopyrrolidin-3-yl alaninate hydrochloride. The intermediate was prepared in a manner similar to that described for Intermediate 13.

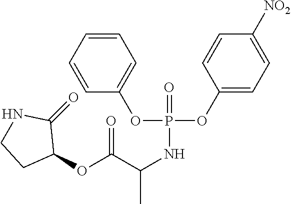 [SMILES: CC(NP(=O)(Oc1ccccc1)Oc1ccc([N+](=O)[O-])cc1)C(=O)O[C@H]1CCNC1=O]

(S)-2-oxopyrrolidin-3-yl ((4-nitrophenoxy)(phenoxy)phosphoryl) alaninate. The intermediate was prepared in a manner similar to that described for Intermediate 35. ¹H NMR (400 MHz, DMSO-d₆) δ 8.34-8.24 (m, 2H), 8.14-8.03 (m, 1H), 7.57-7.36 (m, 3H), 7.33-7.19 (m, 2H), 6.84-6.69 (m, 1H), 5.25-5.09 (m, 1H), 4.14-3.97 (m, 1H), 3.25-3.11 (m, 2H), 2.45-2.30 (m, 1H), 1.87-1.72 (m, 1H), 1.32-1.21 (m, 3H). ³¹P NMR (162 MHz, DMSO-d₆) δ −1.247 (s), −1.520 (s), −1.581 (s). MS m/z=898.62 [M+1].

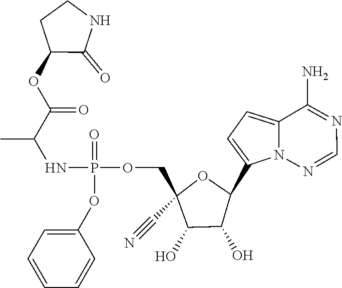 [SMILES: CC(NP(=O)(OC[C@@]1(C#N)O[C@@H](c2ccc3c(N)ncnn23)[C@H](O)[C@@H]1O)Oc1ccccc1)C(=O)O[C@H]1CCNC1=O]

(S)-2-oxopyrrolidin-3-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate. Intermediate 4 (54 mg, 0.163 mmol), intermediate (S)-2-oxopyrrolidin-3-yl ((4-nitrophenoxy)(phenoxy)phosphoryl) alaninate (88 mg, 0.196 mmol) and MgCl₂ (62 mg, 0.652 mmol) were taken up in THF (3 mL). After 5 minutes the resulting mixture was placed in a 50° C. bath. After stirring for 10 minutes DIPEA (0.071 mL, 0.407 mmol) was added in a dropwise manner. The reaction was run until the intermediate was consumed as determined by LC/MS. The reaction was cooled to room temperature and quenched via the addition of a saturated aqueous solution of NaHCO₃. The layers were separated and the aqueous layer was washed with DCM (3×). The combined organic layers were washed with brine and dried over Na₂SO₄. After removal of the drying agent by vacuum filtration, the filtrate was concentrated and the intermediate acetonide protected product (48 mg) was isolated by HPLC. The intermediate acetonide protected product was dissolved in THF (2.5 mL), the resulting solution was cooled in an ice bath, and a 12 N solution of HCl in water (0.3 mL) was added dropwise. The reaction progress was monitored by LC/MS. Upon reaction completion the reaction was concentrated, the residue was combined with that from another reaction ran on the exact same scale and the product was isolated from the combined residues by reverse phase HPLC. ¹H NMR (400 MHz, Methanol-d₄, chemical shift with asterisk (*) denotes shift of associated proton(s) on another isomer) δ 7.81 (s, 0.6H), 7.80 (s, 0.4H), 7.37-7.27 (m, 2H), 7.26-7.14 (m, 3H), 6.87-6.83 (m, 1H), 6.78-6.72 (m, 1H), 5.54-5.48 (m, 1H), 5.33-5.20 (m, 1H), 4.67-4.59 (m, 1H), 4.54-4.30 (m, 3H), 4.07-3.88 (m, 1H), 3.40-3.24 (m, 2H), 2.58-2.37 (m, 1H), 2.03-1.88 (m, 1H), 1.31 (d, J=7.2 Hz, 2.13H), 1.26 (d, J=7.1 Hz, 0.87H). ³¹P NMR (162 MHz, Methanol-d₄) δ 3.233 (s), 3.099 (s), 2.896 (s). MS m/z=602.08 [M+1].

### Example 154. 3,3-dimethylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

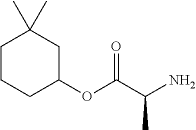

3,3-dimethylcyclohexyl L-alaninate. The intermediate was prepared from Cbz-L-alaninate (1.2 g, 5.38 mmol) and 3,3-dimethylcyclohexanol (2.1 g, 16.13 mmol, racemic mixture) in a manner similar to that described for Intermediate 26. MS m/z=200 [M+H].

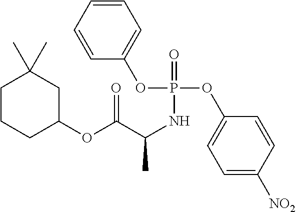 [SMILES: C[C@H](NP(=O)(Oc1ccccc1)Oc1ccc([N+](=O)[O-])cc1)C(=O)OC1CCCC(C)(C)C1]

3,3-dimethylcyclohexyl ((4-nitrophenoxy)(phenoxy)phosphoryl)-L-alaninate. The intermediate was prepared as isomeric mixture (1:1:1:1) from 3,3-dimethylcyclohexyl L-alaninate (1.0 g, 5.02 mmol) in a manner similar to that described for Intermediate 25. ¹H NMR (400 MHz, Chloroform-d) δ 8.27-8.18 (m, 2H), 7.40-7.31 (m, 4H), 7.27-7.21 (m, 3H), 4.93-4.80 (m, 1H), 4.08 (m, 1H), 3.91 (m, 1H), 1.78-1.41 (m, 3H), 1.39 (m, 3H), 1.36-1.04 (m, 5H), 0.96-0.90 (m, 6H). ³¹P NMR (162 MHz, Chloroform-d) δ −3.00, −3.03, −3.06, −3.08. MS m/z=477 [M+H].

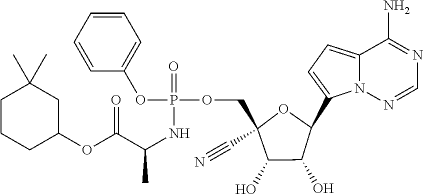 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCC(C)(C)C1]

3,3-dimethylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was prepared from Intermediate 4 (60 mg, 0.18 mmol) and 3,3-dimethylcyclohexyl ((4-nitrophenoxy)(phenoxy)phosphoryl)-L-alaninate (129 mg, 0.27 mmol) in a manner similar to that described for Example 3. ¹H NMR (400 MHz, Methanol-d4) δ 7.79 (m, 1H), 7.38-7.10 (m, 5H), 6.84 (m, 1H), 6.73 (m, 1H), 5.55-5.44 (m, 1H), 4.78 (m, 1H), 4.67-4.55 (m, 1H), 4.54-4.30 (m, 3H), 3.95-3.74 (m, 1H), 1.83 (s, 1H), 1.65-1.35 (m, 3H), 1.34-0.98 (m, 7H), 0.97-0.82 (m, 6H). ³¹P NMR (162 MHz, Methanol-d4) δ 3.30, 3.27. MS m/z=629 [M+H].

### Example 155. trans-2-methylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

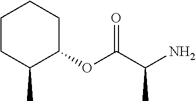

trans-2-methylcyclohexyl L-alaninate. The intermediate was prepared from Cbz-L-alaninate (1.0 g, 4.48 mmol) and trans-2-methylcyclohexanol (2.2 g, 17.9 mmol, racemic mixture) in a manner similar to that described for Intermediate 26. MS m/z=186 [M+H].

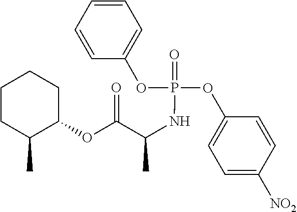 [SMILES: C[C@H](NP(=O)(Oc1ccccc1)Oc1ccc([N+](=O)[O-])cc1)C(=O)O[C@H]1CCCC[C@@H]1C]

trans-2-methylcyclohexyl ((4-nitrophenoxy)(phenoxy)phosphoryl)-L-alaninate. The intermediate was prepared as isomeric mixture from trans-2-methylcyclohexyl L-alaninate (590 mg, 2.66 mmol) in a manner similar to that described for Intermediate 25. ¹H NMR (400 MHz, Chloroform-d) δ 8.27-8.18 (m, 2H), 7.45-7.32 (m, 4H), 7.30-7.15 (m, 3H), 4.43 (m, 1H), 4.28-4.03 (m, 1H), 3.90 (m, 1H), 1.88 (m, 1H), 1.75 (m, 2H), 1.69-1.47 (m, 2H), 1.41 (m, 3H), 1.38-1.13 (m, 3H), 1.12-0.98 (m, 1H), 0.84 (m, 3H). ³¹P NMR (162 MHz, Chloroform-d) δ −2.99, −3.00, −3.05, −3.08. MS m/z=463 [M+H].

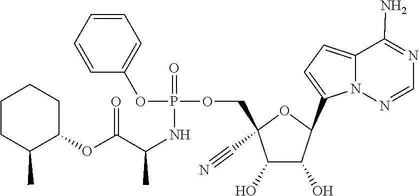 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CCCC[C@@H]1C]

trans-2-methylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was prepared from Intermediate 4 (83 mg, 0.25 mmol) and trans-2-methylcyclohexyl ((4-nitrophenoxy)(phenoxy)phosphoryl)-L-alaninate (174 mg, 0.38 mmol) in a manner similar to that described for Example 3. ¹H NMR (400 MHz, Methanol-d4) δ 7.79 (m, 1H), 7.31 (m, 2H), 7.19 m, 3H), 6.85 (m, 1H), 6.73 m, 1H), 5.56-5.46 (m, 1H), 4.62 (m, 1H), 4.53-4.39 (m, 2H), 4.38-4.25 (m, 2H), 3.96-3.78 (m, 1H), 1.85 (m, 1H), 1.79-1.38 (m, 4H), 1.36-1.11 (m, 6H), 1.12-0.90 (m, 1H), 0.89-0.70 (m, 3H). ³¹P NMR (162 MHz, Methanol-d4) δ 3.32, 3.29, 3.28, 3.27. MS m/z=615 [M+H].

### Example 156. 2,2-dimethyltetrahydro-2H-pyran-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

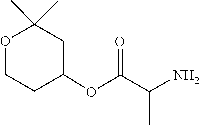

2,2-dimethyltetrahydro-2H-pyran-4-yl alaninate. The intermediate was prepared from Cbz-L-alaninate (1.8 g, 8.06 mmol) and 2,2-dimethyl-4-hydroxytetrahydro-2H-pyran (1.36 mL, 9.68 mmol, racemic mixture) in a manner similar to that described for Intermediate 26. ¹H NMR (400 MHz, Chloroform-d) δ 5.08 (m, 1H), 3.90-3.76 (m, 1H), 3.69 (m, 1H), 3.55 (m, 1H), 2.10-1.74 (m, 4H), 1.67-1.39 (m, 2H), 1.34 (dd, J=7.0, 0.9 Hz, 3H), 1.27 (s, 3H), 1.24 (s, 3H). MS m/z=202 [M+H].

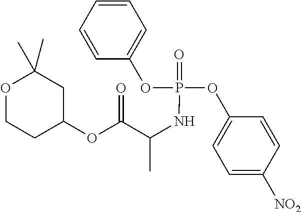 [SMILES: CC(NP(=O)(Oc1ccccc1)Oc1ccc([N+](=O)[O-])cc1)C(=O)OC1CCOC(C)(C)C1]

2,2-dimethyltetrahydro-2H-pyran-4-yl ((4-nitrophenoxy)(phenoxy)phosphoryl)alaninate. The intermediate was prepared as isomeric mixture from 2,2-dimethyltetrahydro-2H-pyran-4-yl alaninate (1.10 g, 5.47 mmol) in a manner similar to that described for Intermediate 25. ¹H NMR (400 MHz, Chloroform-d) δ 8.28-8.14 (m, 2H), 7.44-7.27 (m, 3H), 7.27-7.10 (m, 4H), 5.10-4.97 (m, 1H), 4.18-4.02 (m, 1H), 3.91-3.73 (m, 2H), 3.71-3.59 (m, 1H), 1.89-1.74 (m, 2H), 1.75-1.46 (m, 2H), 1.40 m, 3H), 1.23 (m, 6H). ³¹P NMR (162 MHz, Chloroform-d) δ −3.15, −3.13. MS m/z=479 [M+H].

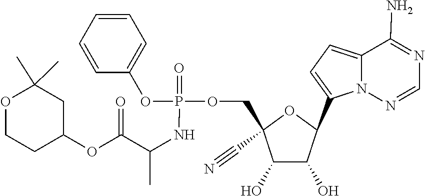 [SMILES: CC(NP(=O)(OC[C@@]1(C#N)O[C@@H](c2ccc3c(N)ncnn23)[C@H](O)[C@@H]1O)Oc1ccccc1)C(=O)OC1CCOC(C)(C)C1]

2,2-dimethyltetrahydro-2H-pyran-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate. The product was prepared from Intermediate 4 (55 mg, ×0.17 mmol) and 2,2-dimethyltetrahydro-2H-pyran-4-yl ((4-nitrophenoxy)(phenoxy)phosphoryl)alaninate (119 mg, 0.25 mmol) in a manner similar to that described for Example 3. ¹H NMR (400 MHz, Methanol-d4) δ 7.85-7.72 (m, 1H), 7.36-7.07 (m, 5H), 6.85 (m, 1H), 6.80-6.65 (m, 1H), 5.51 (m, 1H), 5.06-4.92 (m, 1H), 4.68-4.54 (m, 1H), 4.54-4.29 (m, 3H), 3.97-3.80 (m, 1H), 3.77-3.54 (m, 2H), 1.88-1.72 (m, 2H), 1.41 (m, 2H), 1.29-1.13 (m, 9H). ³¹P NMR (162 MHz, Methanol-d4) δ 3.37, 3.21, 3.19, 2.97. MS m/z=631 [M+H].

### Example 157. neopentyl (2S)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate

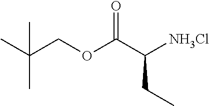

neopentyl (S)-2-aminobutanoate hydrochloride. Chlorotrimethylsilane (1.23 mL, 10.0 mmol) was added to a solution of (S)-2-aminobutanoic acid (1 g, 10 mmol) in neopentyl alcohol (8.54 g) and the resulting mixture was heated to 80° C. After 21 h, the reaction mixture was concentrated under reduced pressure at 70° C. The crude solid residue was taken up into hexanes (150 mL) and was stirred for 4 h. The resulting solid was collected by vacuum filtration to afford the intermediate. ¹H NMR (400 MHz, methanol-d₄) δ 4.05 (t, J=6.1 Hz, 1H), 3.96 (qd, J=10.5, 0.9 Hz, 2H), 2.08-1.88 (m, 2H), 1.07 (td, J=7.6, 0.9 Hz, 3H), 0.99 (s, 9H).

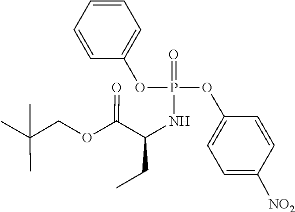 [SMILES: CC[C@H](NP(=O)(Oc1ccccc1)Oc1ccc([N+](=O)[O-])cc1)C(=O)OCC(C)(C)C]

neopentyl (2S)-2-(((4-nitrophenoxy)(phenoxy)phosphoryl)amino)butanoate. To a solution of neopentyl (S)-2-aminobutanoate hydrochloride (0.994 g, 4.74 mmol) and phenyl dichlorophosphate (0.705 mL, 4.74 mmol) in dichloromethane (23 mL) was added triethylamine (1.2 mL, 9.4 mmol) at 0° C. under and argon atmosphere. The resulting mixture was allowed to warm to RT and was stirred for 1.5 h. 4-Nitrophenol (660 mg, 4.74 mmol) and triethylamine (0.66 mL, 4.7 mmol) were then added. After 1 h, the reaction mixture was diluted with dichloromethane (50 mL) and the resulting mixture was washed with saturated aqueous sodium bicarbonate solution (50 mL) and brine (50 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, CDCl₃) δ 8.22 (d, J=8.3 Hz, 2H), 7.47-7.29 (m, 4H), 7.28-7.15 (m, 3H), 4.18-4.03 (m, 1H), 3.94-3.72 (m, 3H), 1.90-1.69 (m, 2H), 0.97-0.82 (m, 12H). ³¹P NMR (162 MHz, CDCl₃) δ −2.64 (s), −2.70 (s). MS m/z=450.96 [M+1].

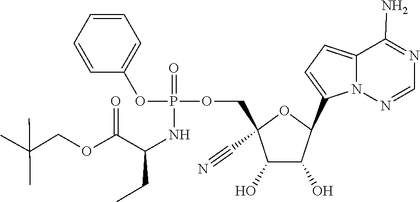 [SMILES: CC[C@H](NP(=O)(OC[C@@]1(C#N)O[C@@H](c2ccc3c(N)ncnn23)[C@H](O)[C@@H]1O)Oc1ccccc1)C(=O)OCC(C)(C)C]

neopentyl (2S)-2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)butanoate. To a mixture of Intermediate 4 (34.0 mg, 0.102 mmol), neopentyl (2S)-2-(((4-nitrophenoxy)(phenoxy)phosphoryl)amino)butanoate (46.1 mg, 0.102 mmol), and magnesium chloride (9.7 mg, 0.102 mmol) was added acetonitrile (0.50 mL) at RT. The resulting mixture was warmed to 50° C., and was allowed to stir for 5 min. N,N-Diisopropylethylamine (0.045 mL, 0.256 mmol) was then added and the resulting mixture was stirred at 50° C. for 1 h. The reaction mixture was then allowed to cool to RT, and concentrated aqueous hydrochloric acid solution (12 M, 0.119 mL, 1.43 mmol) was added. After 1 h, the reaction mixture was diluted with saturated aqueous sodium carbonate solution (20 mL) and ethyl acetate (20 mL). The layers were split and the organic layer was washed with brine (20 mL), was dried over anhydrous sodium sulfate, and was concentrated under reduced pressure. The crude residue was purified by preparatory HPLC (Phenominex Luna 5 u C18(2) 100 Å 100×30 mm column, 5-100% acetonitrile/water gradient) to afford the product. ¹H NMR (400 MHz, methanol-d₄) δ 7.79 (s, 0.6H), 7.78 (s, 0.4H), 7.35-7.10 (m, 5H), 6.86-6.82 (m, 1H), 6.75-6.71 (m, 1H), 5.53-5.47 (m, 1H), 4.65-4.58 (m, 1H), 4.52-4.30 (m, 3H), 3.87-3.63 (m, 3H), 1.80-1.54 (m, 2H), 0.93-0.81 (m, 12H). ³¹P NMR (162 MHz, methanol-d₄) δ 3.61 (s) 3.57 (s). LCMS: MS m/z=603.30 [M+1], t_{R}=1.59 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.88 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=5.66 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

Resolution of the Sp and Rp diastereomers. The product was purified via chiral preparatory SFC (SFC ID 5 um 4.6×150 mm column, SFC 30% IPA) to afford the diastereomers:

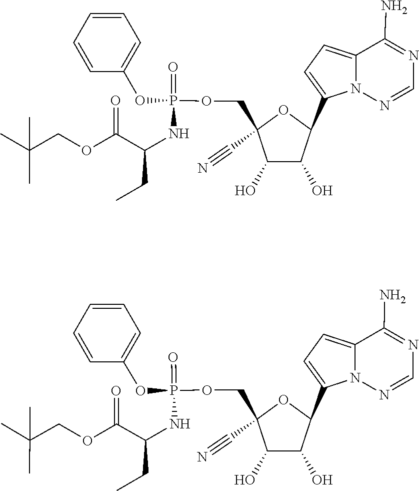 [SMILES: CC[C@H](N[P@@](=O)(OC[C@@]1(C#N)O[C@@H](c2ccc3c(N)ncnn23)[C@H](O)[C@@H]1O)Oc1ccccc1)C(=O)OCC(C)(C)C ; CC[C@H](N[P@](=O)(OC[C@@]1(C#N)O[C@@H](c2ccc3c(N)ncnn23)[C@H](O)[C@@H]1O)Oc1ccccc1)C(=O)OCC(C)(C)C]

### Example 158. First Eluting Diastereomer

¹H NMR (400 MHz, methanol-d₄) δ 7.78 (s, 1H), 7.34-7.23 (m, 2H), 7.19-7.11 (m, 3H), 6.84 (d, J=4.5 Hz, 1H), 6.73 (d, J=4.5 Hz, 1H), 5.50 (d, J=5.1 Hz, 1H), 4.61 (t, J=5.3 Hz, 1H), 4.52-4.43 (m, 2H), 4.36 (dd, J=10.8, 5.3 Hz, 1H), 3.86-3.70 (m, 3H), 1.82-1.69 (m, 1H), 1.69-1.54 (m, 1H), 0.91 (s, 9H), 0.85 (t, J=7.4 Hz, 3H). ³¹P NMR (162 MHz, methanol-d₄) δ 3.58 (s). LCMS: MS m/z=603.30 [M+1], t_{R}=1.57 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.88 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 m/min. HPLC: t_{R}=5.66 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 159. Second Eluting Diastereomer

¹H NMR (400 MHz, methanol-d₄) δ 7.83 (s, 1H), 7.38-7.27 (m, 2H), 7.25-7.14 (m, 3H), 6.93 (d, J=4.5 Hz, 1H), 6.77 (d, J=4.6 Hz, 1H), 5.49 (d, J=5.0 Hz, 1H), 4.59 (t, J=5.3 Hz, 1H), 4.48-4.38 (m, 2H), 4.34 (dd, J=10.9, 5.5 Hz, 1H), 3.86-3.78 (m, 1H), 3.76 (d, J=10.5 Hz, 1H), 3.66 (d, J=10.5 Hz, 1H), 1.80-1.56 (m, 2H), 0.90-0.82 (m, 12H). ³¹P NMR (162 MHz, methanol-d₄) δ 3.62 (s). LCMS: MS m/z=603.30 [M+1], t_{R}=1.59 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.88 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=5.66 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 160. (S)-1-amino-1-oxopropan-2-yl ((((2R,3S,4R,5S)-5-(4-aminopyrrolo[2,1-f]triazin-7-yl)-2-cyano-3,4-dihydroxytetrahydrofuran-2-yl)methoxy)(phenoxy) phosphoryl)-L-alaninate

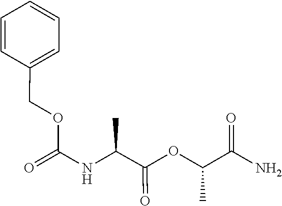 [SMILES: C[C@H](NC(=O)OCc1ccccc1)C(=O)O[C@@H](C)C(N)=O]

(S)-1-amino-1-oxopropan-2-yl ((benzyloxy)carbonyl)-L-alaninate. Cbz-L-Ala (446 mg, 2 mmol) was dissolved in anhydrous acetonitrile (15 mL). EDCI (460 mg, 2.4 mmol) was added in one portion, and the reaction was stirred for 15 mins. (S)-Lactamide (178 mg, 2 mmol) was added in one portion and then DMAP (269 mg, 2.2 mmol) was added. Reaction was stirred for 6 hrs. Reaction was diluted with EtOAc (30 mL) and washed with 5% aqueous citric acid solution (15 mL), followed with saturated aqueous sodium bicarbonate solution (15 mL) and finally with brine (10 mL). Organic extract was dried over anhydrous sodium sulfate and concentrated under reduced pressure to give the intermediate which was used for next step without purification. ¹H NMR (400 MHz, Chloroform-d) δ 7.35 (m, 5H), 6.55 (s, 1H), 5.38-4.93 (m, 5H), 4.34 (p, J=7.2 Hz, 1H), 1.47 (m, 6H).

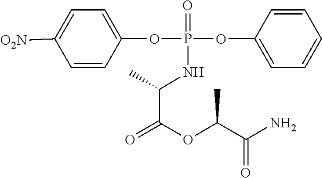 [SMILES: C[C@H](NP(=O)(Oc1ccccc1)Oc1ccc([N+](=O)[O-])cc1)C(=O)O[C@@H](C)C(N)=O]

(S)-1-amino-1-oxopropan-2-yl ((4-nitrophenoxy)(phenoxy)phosphoryl)-L-alaninate. (S)-1-amino-1-oxopropan-2-yl ((benzyloxy)carbonyl)-L-alaninate (479 mg, 1.63 mmol) was dissolved in anhydrous THF (25 mL). 10% Pd/C Degussa type was added, and the mixture was stirred under atmospheric hydrogen for 3 hrs. Catalyst was filtered off and washed with anhydrous THF (5 mL). Filtrate was concentrated under reduced pressure, and the resulting material was used without purification. Phenyl dichlorophosphate (242 uL, 1.63 mmol) was dissolved in anhydrous DCM (15 mL) and stirred under atmospheric nitrogen in an ice bath. Above prepared material was mixed with anhydrous THF (5 mL) and added to the reaction in several portions over 15 mins. Reaction was stirred for 1 hr. Triethylamine (250 uL, 1.79 mmol) was added to the reaction mixture dropwise. Reaction was stirred for 1 hr. More triethylamine (250 uL, 1.79 mmol) was added to the reaction mixture dropwise. Reaction was stirred for 45 mins. p-Nitrophenol (181 mg, 1.3 mmol) was added to the reaction in one portion. Ice bath was removed, and the reaction mixture was stirred for 14 hrs. Reaction was diluted with DCM (20 mL) and washed with water (3×20 mL). Dried organic extract over anhydrous sodium sulfate and concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, Chloroform-d) δ 8.29-8.15 (m, 2H), 7.47-7.29 (m, 4H), 7.29-7.14 (m, 3H), 6.52 (m, 1H), 5.56 (s, 1H), 5.17 (m, 1H), 4.20 (m, 1H), 4.07-3.96 (m, 1H), 1.53-1.40 (m, 6H). ³¹P NMR (162 MHz, Chloroform-d) δ −2.53 (s), −2.79 (s). MS m/z=438.0 [M+1]; 436.0 [M−1].

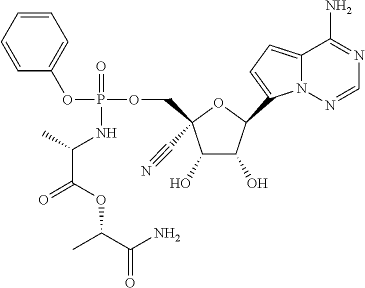 [SMILES: CC(OP(=O)(N[C@@H](C)C(=O)O[C@@H](C)C(N)=O)Oc1ccccc1)[C@@]1(C#N)O[C@@H](c2ccc3c(N)ncnn23)[C@H](O)[C@@H]1O]

(S)-1-amino-1-oxopropan-2-yl ((((2R,3S,4R,5S)-5-(4-aminopyrrolo[2,1-f]triazin-7-yl)-2-cyano-3,4-dihydroxytetrahydrofuran-2-yl)methoxy)(phenoxy) phosphoryl)-L-alaninate. Intermediate 4 (50 mg, 0.15 mmol) and (S)-1-amino-1-oxopropan-2-yl ((4-nitrophenoxy)(phenoxy)phosphoryl)-L-alaninate (79 mg, 0.18 mmol) were mixed in anhydrous THF (3 mL). Magnesium chloride (36 mg, 0.375 mmol) was added in one portion. Reaction was stirred at 50° C. for 30 mins. DIPEA (65 uL, 0.375 mmol) was added, and the reaction was stirred for 14 hrs at 50° C.

Reaction was cooled to RT, diluted with EtOAc (15 mL) and washed with 2% aqueous sodium carbonate solution (2×10 mL) and followed with brine (10 mL). Organic extract was dried over anhydrous sodium sulfate and concentrated under reduced pressure. The crude residue was purified via SiO₂ column chromatography (4 g SiO₂ Combiflash HP Gold Column, 0-5-10% methanol/DCM). Fractions having the desired product were combined and concentrated under reduced pressure. Resulting oil was dissolved in MeCN (2 mL) and stirred in an ice bath. 12 M HCl(aq) (300 uL) was added dropwise to the reaction and then stirred for 1 hr. Reaction was diluted with EtOAc (10 mL). Saturated aqueous sodium bicarbonate solution was added to give of pH 8. Organic layer was collected, washed with brine (10 mL), dried over anhydrous sodium sulfate and concentrated under reduced pressure. The crude residue was purified via SiO₂ column chromatography (4 g SiO₂ Combiflash HP Gold Column, 0-10-20% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.79 (m, 1H), 7.38-7.08 (m, 5H), 6.84 (m, 1H), 6.73 (m, 1H), 5.50 (d, J=4.9 Hz, 1H), 5.04-4.93 (m, 1H), 4.67-4.58 (m, 1H), 4.54-4.29 (m, 3H), 3.97 (m, 1H), 1.46-1.25 (m, 6H). ³¹P NMR (162 MHz, Methanol-d4) δ 3.52 (s), 3.39 (s). MS m/z=590.1 [M+1]; 588.0 [M−1].

### Example 161. (4aR,6S,7S,7aS)-6-(4-aminopyrrolo[2,1-f][1,2,4]triazin-7-yl)-2-(cyclopentyloxy)-7-hydroxydihydro-4H-furo[3,2-d][1,3,2]dioxaphosphinine-4a(6H)-carbonitrile 2-oxide

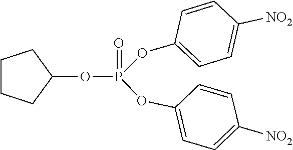 [SMILES: O=[N+]([O-])c1ccc(OP(=O)(Oc2ccc([N+](=O)[O-])cc2)OC2CCCC2)cc1]

Cyclopentyl bis(4-nitrophenyl) phosphate. To a solution of cyclopentanol (0.87 g, 10.1 mmol) and phosphorus oxychloride (1.56 g, 10.17 mmol) in dichloromethane (15 mL) was added triethylamine (1.4 mL, 10.1 mmol) at −78° C. under argon atmosphere. The resulting mixture was allowed to warm to 0° C. and stirred for 0.5 h. 4-Nitrophenol (2.65 g, 19.05 mmol) and triethylamine (2.8 mL, 20.2 mmol) were then added. After 1 h, the reaction mixture was diluted with hexane (15 mL) and the resulting mixture was filtered. The filtrate was purified by silica gel column eluted with 20-33% ethyl acetate/hexanes to afford the intermediate. ¹H NMR (400 MHz, CDCl₃) δ 8.26 (d, J=9.6 Hz, 4H), 7.39 (d, J=9.6 Hz, 4H), 5.15-5.25 (m, 1H), 1.60-2.0 (m, 8H). ³¹P NMR (162 MHz, CDCl₃) δ −14.2 (s).

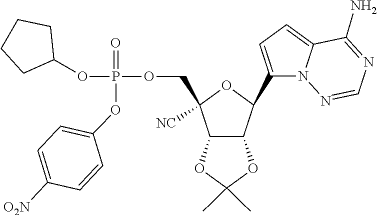 [SMILES: CC1(C)O[C@H]2[C@H](c3ccc4c(N)ncnn34)O[C@](C#N)(COP(=O)(Oc3ccc([N+](=O)[O-])cc3)OC3CCCC3)[C@H]2O1]

((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclopentyl (4-nitrophenyl) phosphate. To a mixture of Intermediate 4 (105 mg, 0.317 mmol), cyclopentyl bis(4-nitrophenyl) phosphate (140 mg, 0.343 mmol), and magnesium chloride (54 mg, 0.567 mmol) in THF (2.0 mL) was added N,N-Diisopropylethylamine (115 μL, 0.66 mmol). The resulting mixture was stirred at RT for 16 h. The reaction mixture was diluted with ethyl acetate (5 mL), washed with water, dried over MgSO₄ and concentrated under reduced pressure. The crude residue was purified by silica gel column eluted with 30-100% ethyl acetate/hexane to afford the intermediate. ¹H NMR (400 MHz, CDCl3) δ 8.09 (d, J=8.8 Hz, 1H), 7.93 (d, J=9.6 Hz, 1H), 7.86 (d, J=5.2 Hz, 1H), 7.3 (d, J=9.2 Hz, 1H), 7.2 (d, J=9.6 Hz, 1H), 6.65-6.71 (m, 1H), 6.55-6.59 (m, 1H), 5.8 (brs, 2H), 5.58-5.63 (m, 1H), 5.2-5.3 (m, 1H), 5.0-5.15 (m, 2H), 4.38-4.52 (m, 2H), 1.5-2.0 (m, 11H), 1.35-1.37 (2s, 3H). ³¹P NMR (162 MHz, CDCl₃) δ −8.84 (s), −9.13 (s). MS m/z=601.0 [M+H].

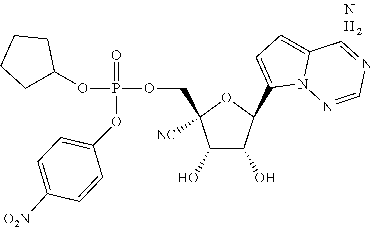 [SMILES: N#C[C@]1(COP(=O)(Oc2ccc([N+](=O)[O-])cc2)OC2CCCC2)O[C@@H](c2ccc3cncnn23)[C@H](O)[C@@H]1O]

((2R,3S,4R,5S)-5-(4-aminopyrrolo[2,1-f][1,2,4]triazin-7-yl)-2-cyano-3,4-dihydroxytetrahydrofuran-2-yl)methyl cyclopentyl (4-nitrophenyl) phosphate. To a solution of ((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methyl cyclopentyl (4-nitrophenyl) phosphate (120 mg, 0.20 mmol) in acetonitrile (1.2 mL) and water (0.2 mL) was added HCl (0.2 mL, 37%) at 0° C. The reaction mixture was stirred at RT for 3 h. The reaction mixture was quenched with NaHCO₃ (210 mg), stirred for 1 h and concentrated. The residue was treated with CH₃CO₂Et, washed with water, dried over anhydrous sodium sulfate, filtered, concentrated and dried in high vacuum to afford the intermediate. ¹H NMR (400 MHz, CDCl₃+5% CD₃OD) δ 8.09 (d, J=8.8 Hz, 1H), 7.95 (d, J=8.6 Hz, 1H), 7.74 (d, J=10 Hz, 1H), 7.27 (d, J=7.6 Hz, 1H), 7.19 (d, J=9.6 Hz, 1H), 6.55-6.7 (m, 2H), 4.95-5.1 (m, 1H), 4.3-4.55 (m, 4H), 1.45-1.95 (m, 8H). ³¹P NMR (162 MHz, CDCl₃+5% CD₃OD) δ −8.88 (s), −9.06 (s). MS m/z=561.0 [M+H].

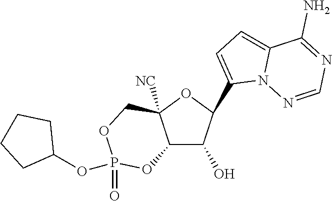

(4aR,6S,7S,7aS)-6-(4-aminopyrrolo[2,1-f][1,2,4]triazin-7-yl)-2-(cyclopentyloxy)-7-hydroxydihydro-4H-furo[3,2-d][1,3,2]dioxaphosphinine-4a(6H)-carbonitrile 2-oxide. The mixture of ((2R,3S,4R,5S)-5-(4-aminopyrrolo[2,1-f][1,2,4]triazin-7-yl)-2-cyano-3,4-dihydroxytetrahydrofuran-2-yl)methyl cyclopentyl (4-nitrophenyl) phosphate (55 mg, 0.098 mmol) and DMAP (110 mg, 0.9 mmol) in ethyl acetate (10 mL) was stirred at 85° C. for 36 h. The reaction mixture was concentrated under reduced pressure. The crude residue was purified by preparatory HPLC (Phenominex Synergi 4 u Hydro-RR 80 Å 150×30 mm column, 40-100% acetonitrile/water gradient) to afford the intermediate. ¹H NMR (400 MHz, CD₃CN) δ 7.86 (s, 1H), 6.78-6.81 (m, 2H), 6.43 (brs, 2H), 5.63 (brs, 1H), 5.26-5.29 (m, 1H), 5.0-5.1 (m, 2H), 4.84 (d, J=5.6 Hz, 1H), 4.68-4.77 (m, 1H), 4.44 (d, J=10.4 Hz, 1H), 4.25 (brs, 1H), 1.6-2.0 (m, 8H). ³¹P NMR (162 MHz, CD₃CN) δ −8.3 (s). MS m/z=422.3 [M+H].

### Example 162. 2-(dimethylamino)ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

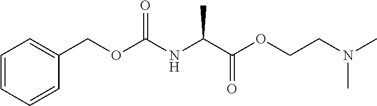 [SMILES: C[C@H](NC(=O)OCc1ccccc1)C(=O)OCCN(C)C]

2-(dimethylamino)ethyl ((benzyloxy)carbonyl)-L-alaninate. Cbz-L-Ala (446 mg, 2 mmol) was dissolved in anhydrous DMF (10 mL). Triethylamine (698 uL, 5 mmol) was added in one portion. 2-chloro-N,N-dimethylethanamine hydrochloride (317 mg, 2.2 mmol) was added. The reaction was stirred for 2 hrs. DMAP (24 mg, 0.2 mmol) was added, and the reaction was stirred for 2 hrs. Reaction was warmed to 50° C. and stirred for 2 hrs. More triethylamine (700 uL) and 2-chloro-N,N-dimethylethanamine hydrochloride (317 mg) were added. The reaction was stirred at 50° C. for 16 hrs.

Reaction was cooled to RT, diluted with EtOAc (30 mL) and washed with saturated aqueous sodium bicarbonate solution (2×15 mL) and followed with brine (10 mL). Organic extract was dried over anhydrous sodium sulfate and concentrated under reduced pressure. The crude residue was purified via SiO₂ column chromatography (24 g SiO₂ Combiflash HP Gold Column, 0-10% methanol/DCM) to afford the intermediate. ¹H NMR (400 MHz, Chloroform-d) δ 7.41-7.27 (m, 5H), 5.37 (m, 1H), 5.11 (s, 2H), 4.50-4.33 (m, 1H), 4.26 (m, 2H), 2.59 (m, 2H), 2.29 (s, 6H), 1.42 (d, J=7.2 Hz, 3H).

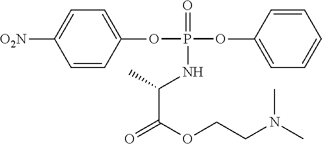 [SMILES: C[C@H](NP(=O)(Oc1ccccc1)Oc1ccc([N+](=O)[O-])cc1)C(=O)OCCN(C)C]

2-(dimethylamino)ethyl ((4-nitrophenoxy)(phenoxy)phosphoryl)-L-alaninate. 2-(dimethylamino)ethyl ((benzyloxy)carbonyl)-L-alaninate (310 mg, 1.05 mmol) was dissolved in anhydrous THF (15 mL). 10% Pd/C Degussa type was added, and the mixture was stirred under atmospheric hydrogen for 3 hrs. Catalyst was filtered off and washed with anhydrous THF (5 mL). Filtrate was concentrated under reduced pressure, and the resulting material was used without purification. Phenyl dichlorophosphate (157 uL, 1.05 mmol) was dissolved in anhydrous THF (10 mL) and stirred under atmospheric nitrogen in an ice bath. Above prepared material was dissolved with anhydrous THF (3 mL) and added to the reaction dropwise over 5 mins. Reaction was stirred for 1 hr. Triethylamine (322 uL, 2.32 mmol) was added to the reaction mixture dropwise. Reaction was stirred for 1 hr. p-Nitrophenol (117 mg, 0.84 mmol) was added to the reaction in one portion. Ice bath was removed, and the reaction mixture was stirred for 14 hrs. Reaction was diluted with EtOAc (20 mL) and washed with water (2×20 mL). Dried organic extract over anhydrous sodium sulfate and concentrated under reduced pressure. The crude residue was purified via SiO₂ column chromatography (24 g SiO₂ Combiflash HP Gold Column, 0-3-7% methanol/DCM) to afford the intermediate. ¹H NMR (400 MHz, Chloroform-d) δ 8.22 (m, 2H), 7.46-7.29 (m, 4H), 7.29-7.14 (m, 3H), 4.33-4.02 (m, 4H), 2.58 (m, 2H), 2.29 (s, 6H), 1.42 (m, 3H). ³¹P NMR (162 MHz, Chloroform-d) δ −3.04 (s). MS m/z=438.1 [M+1]; 436.1 [M−1].

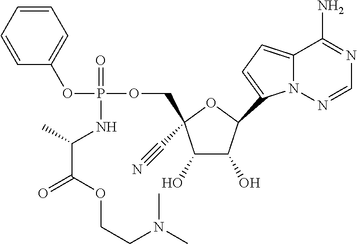 [SMILES: C[C@H](NP(=O)(OC[C@@]1(C#N)O[C@@H](c2ccc3c(N)ncnn23)[C@H](O)[C@@H]1O)Oc1ccccc1)C(=O)OCCN(C)C]

2-(dimethylamino)ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.15 mmol) and 2-(dimethylamino)ethyl ((4-nitrophenoxy)(phenoxy)phosphoryl)-L-alaninate (79 mg, 0.18 mmol) were mixed in anhydrous THF (3 mL). Magnesium chloride (43 mg, 0.45 mmol) was added in one portion. Reaction was stirred for 10 mins. DIPEA (52 uL, 0.3 mmol) was added, and the reaction was stirred for 4 hrs. More magnesium chloride (90 mg, 0.9 mmol) was added, and the reaction was stirred for 16 hrs. Reaction was diluted with EtOAc (15 mL) and washed with 2% aqueous sodium carbonate solution (2×10 mL) and followed with brine (10 mL). Organic extract was dried over anhydrous sodium sulfate and concentrated under reduced pressure. The crude residue was purified via SiO₂ column chromatography (4 g SiO₂ Combiflash HP Gold Column, 0-5-10-20% methanol/DCM). Fractions having the desired product were combined and concentrated under reduced pressure. Resulting oil was dissolved in MeCN (2 mL) and stirred in an ice bath. 12 M HCl(aq) (300 uL) was added dropwise to the reaction and then stirred for 2 hrs. Reaction was diluted with EtOAc (10 mL). Saturated aqueous sodium bicarbonate solution was added to give of pH 8. Organic layer was collected, washed with brine (10 mL), dried over anhydrous sodium sulfate and concentrated under reduced pressure. The crude residue was purified via SiO₂ column chromatography (4 g SiO₂ Combiflash HP Gold Column, 0-10-20% methanol/DCM). Fractions having the desired product were combined and concentrated under reduced pressure and the resulting compound was then dissolved in MeCN and water and freeze-dried to afford the product. ¹H NMR (400 MHz, Acetonitrile-d₃, Water-d2) δ 7.89-7.78 (m, 1H), 7.45-7.29 (m, 2H), 7.29-7.04 (m, 3H), 6.85 (m, 1H), 6.79-6.71 (m, 1H), 5.50 (d, J=4.8 Hz, 1H), 4.66-4.54 (m, 1H), 4.54-4.27 (m, 5H), 3.97 (m, 1H), 3.40-3.24 (m, 2H), 2.88-2.71 (m, 6H), 1.29 (m, 3H). ³¹P NMR (162 MHz, Acetonitrile-d₃, Water-d2) δ 0.93 (s), 0.80 (s). MS m/z=590.2 [M+1]; 588.1 [M−1].

### Example 163. (4aR,6S,7S,7aS)-6-(4-aminopyrrolo[2,1-f][1,2,4]triazin-7-yl)-7-hydroxy-2-isopropoxydihydro-4H-furo[3,2-d][1,3,2]dioxaphosphinine-4a(6H)-carbonitrile 2-oxide

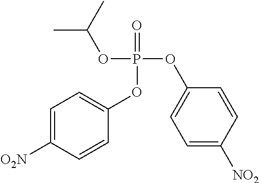 [SMILES: CC(C)OP(=O)(Oc1ccc([N+](=O)[O-])cc1)Oc1ccc([N+](=O)[O-])cc1]

isopropyl bis(4-nitrophenyl) phosphate. To a solution of isopropanol (0.780 mL, 10.2 mmol) and POCl₃ (0.945 mL, 10.2 mmol) in dichloromethane at −78° C. was added triethylamine (1.42 mL, 10.2 mmol). The reaction mixture was stirred at −78° C. for 10 minutes, and was then slowly warmed to 0° C. and stirred for 30 min. A solution of 4-nitrophenol (2.83 g, 20.4 mmol) and triethylamine (2.84 mL, 20.4 mmol) in dichloromethane was then added slowly. The resulting mixture was stirred at 0° C. for 30 min, and was then warmed to RT. After 30 min, the reaction mixture was diluted with hexane (15 mL), and solids were removed by vacuum filtration. The filtrate was purified via SiO₂ column chromatography (120 g SiO₂ Combiflash HP Gold Column, 0-100% ethyl acetate/hexanes) to afford the intermediate. ¹H NMR (400 MHz, CDCl₃) δ 8.27 (d, J=9.1 Hz, 4H), 7.40 (d, J=9.3 Hz, 2H), 4.97 (h, J=6.3 Hz, 1H), 1.42 (d, J=6.2 Hz, 6H). ³¹P NMR (162 MHz, CDCl₃) δ −14.22 (s).

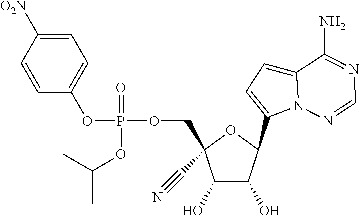 [SMILES: CC(C)OP(=O)(OC[C@@]1(C#N)O[C@@H](c2ccc3c(N)ncnn23)[C@H](O)[C@@H]1O)Oc1ccc([N+](=O)[O-])cc1]

((2R,3S,4R,5S)-5-(4-aminopyrrolo[2,1-f][1,2,4]triazin-7-yl)-2-cyano-3,4-dihydroxytetrahydrofuran-2-yl)methyl isopropyl (4-nitrophenyl) phosphate. To a mixture of Intermediate 4 (167 mg, 0.438 mmol), intermediate isopropyl bis(4-nitrophenyl) phosphate (145 mg, 0.438 mmol), and magnesium chloride (41.7 mg, 0.438 mmol) was added acetonitrile (2.00 mL) at RT. The resulting suspension was warmed to 50° C., and was allowed to stir for 5 min. N,N-Diisopropylethylamine (0.076 mL, 0.438 mmol) was then added and the resulting mixture was stirred at 50° C. for 1 h. The reaction mixture was then allowed to cool to RT, and concentrated aqueous hydrochloric acid solution (12 M, 0.511 mL) was added. After 1 h, the reaction mixture was diluted with saturated aqueous sodium carbonate solution (20 mL) and ethyl acetate (20 mL). The layers were split and the organic layer was washed with brine (20 mL), was dried over anhydrous sodium sulfate, and was concentrated under reduced pressure. The crude residue was purified by preparatory HPLC (Gemini 5 u C18 100 Å 100×30 mm column, 10-100% acetonitrile/water gradient 0.1% TFA). The fractions having the desired product were combined and concentrated to a ˜5 mL volume and saturated aqueous sodium bicarbonate solution was added to neutralize to pH=7. The resulting aqueous mixture was extracted with ethyl acetate (2×10 mL), and the organic extracts were dried over anhydrous sodium sulfate and were concentrated under reduced pressure.

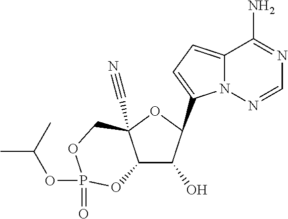

(4aR,6S,7S,7aS)-6-(4-aminopyrrolo[2,1-f][1,2,4]triazin-7-yl)-7-hydroxy-2-isopropoxydihydro-4H-furo[3,2-d][1,3,2]dioxaphosphinine-4a(6H)-carbonitrile 2-oxide. ((2R,3S,4R,5S)-5-(4-aminopyrrolo [2,1-f][1,2,4]triazin-7-yl)-2-cyano-3,4-dihydroxytetrahydrofuran-2-yl)methyl isopropyl (4-nitrophenyl) phosphate (167 mg, 0.438 mmol) was dissolved into ethyl acetate (2 mL) and DMAP (428 mg) was added and the resulting mixture was heated to 80° C. After 24 h, the resulting mixture was concentrated under reduced pressure. The crude residue was purified by preparatory HPLC (Gemini 5 u C18 100 Å 100×30 mm column, 10-100% acetonitrile/water gradient 0.1% TFA) and the fractions having the desired product were combined and concentrated under reduced pressure. The residue was taken up into water/acetonitrile mixture and was neutralized with saturated aqueous sodium bicarbonate solution and was purified via preparatory HPLC (Gemini 5 u C18 100 Å 100×30 mm column, 10-100% acetonitrile/water gradient) to afford the product. 1:0.15 isomer mixture. ¹H NMR (400 MHz, methanol-d₄) δ 7.78 (s, 1H), 6.86 (d, J=4.5 Hz, 1H), 6.78 (d, J=4.5 Hz, 1H), 5.59 (d, J=1.5 Hz, 1H), 5.41 (dd, J=5.4, 2.8 Hz, 1H), 4.84-4.77 (m, 1H), 4.51 (d, J=10.0 Hz, 1H), 1.47 (dd, J=6.2, 3.2 Hz, 6H). ³¹P NMR (162 MHz, methanol-d₄) δ −7.61 (s). MS m/z=396.18 [M+H]. LCMS: MS m/z=396.18 [M+1], t_{R}=1.21 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.12 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

### Example 164. 2-methoxy-2-methylpropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

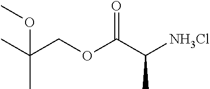

2-methoxy-2-methylpropyl L-alaninate hydrochloride. (tert-butoxycarbonyl)-L-alanine (1.82 mL, 10.0 mmol) and 2-methoxy-2-methylpropan-1-ol (1.00 g, 10.0 mmol) were dissolved in acetonitrile (150 mL). EDCI (1.49 g, 10.0 mmol) and DMAP (1.17 g, 10.0 mmol) were then added and the reaction mixture was stirred at RT. After 2 h, the mixture was concentrated under reduced pressure. The crude residue was purified via SiO₂ column chromatography (25 g SiO₂ Combiflash HP Gold Column, 0-100% ethyl acetate/hexanes). Fractions containing the Boc-protected intermediate were combined and were concentrated under reduced pressure. 4 M HCl in dioxane (20 mL) was added to the concentrate and after 4 h the resulting solids were collected by vacuum filtration to afford the intermediate. ¹H NMR (400 MHz, chloroform-d) δ 4.33-4.20 (m, 1H), 4.17 (d, J=11.3 Hz, 1H) 4.07 (d, J=11.4 Hz, 1H), 3.28-3.18 (m, 3H), 1.75 (d, J=7.1 Hz, 3H), 1.21 (br s, 6H).

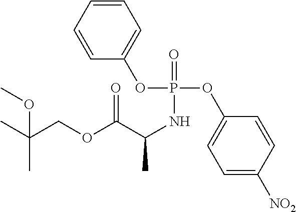 [SMILES: COC(C)(C)COC(=O)[C@H](C)NP(=O)(Oc1ccccc1)Oc1ccc([N+](=O)[O-])cc1]

2-methoxy-2-methylpropyl ((4-nitrophenoxy)(phenoxy)phosphoryl)-L-alaninate. To a solution of 2-methoxy-2-methylpropyl L-alaninate hydrochloride (0.120 g, 0.57 mmol) and phenyl dichlorophosphate (0.084 mL, 0.57 mmol) in dichloromethane (3 mL) was added triethylamine (0.160 mL, 1.13 mmol) at 0° C. under argon atmosphere. The resulting mixture was allowed to warm to RT and was stirred for 1 h. 4-Nitrophenol (0.080 mg, 0.57 mmol) and triethylamine (0.08 mL, 0.57 mmol) were then added. After 1 h, the reaction mixture was diluted with dichloromethane (20 mL) and the resulting mixture was washed with saturated aqueous sodium bicarbonate solution (20 mL) and brine (20 mL), was dried over anhydrous sodium sulfate, and was concentrated under reduced pressure. The crude residue was purified via SiO₂ column chromatography (40 g SiO₂ Combiflash HP Gold Column, 0-100% ethyl acetate/hexanes) to afford the intermediate. ¹H NMR (400 MHz, chloroform-d) δ 8.22 (dd, J=9.3, 2.7 Hz, 2H), 7.44-7.31 (m, 4H), 7.27-7.18 (m, 3H), 4.28-4.12 (m, 1H), 4.08 (d, J=11.4 Hz, 1H), 4.01 (dd, J=11.3, 5.1 Hz, 1H), 3.87 (t, J=10.8 Hz, 1H), 3.20 (d, J=1.3 Hz, 3H), 1.44 (dd, J=7.1, 3.1 Hz, 3H), 1.17 (s, 6H). ³¹P NMR (162 MHz, chloroform-d) δ −3.15 (s), −3.21 (s). MS m/z=453.06 [M+1].

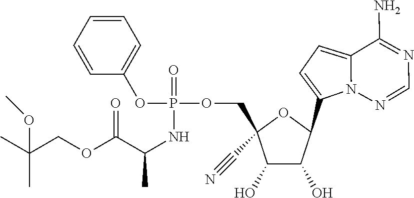 [SMILES: COC(C)(C)COC(=O)[C@H](C)NP(=O)(OC[C@@]1(C#N)O[C@@H](c2ccc3c(N)ncnn23)[C@H](O)[C@@H]1O)Oc1ccccc1]

2-methoxy-2-methylpropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. To a mixture of Intermediate 4 (81.3 mg, 0.245 mmol), intermediate 2-methoxy-2-methylpropyl ((4-nitrophenoxy)(phenoxy)phosphoryl)-L-alaninate (111 mg, 0.245 mmol), and magnesium chloride (23.4 mg, 0.245 mmol) was added acetonitrile (1.20 mL) at RT. The resulting mixture was warmed to 50° C., and was allowed to stir for 5 min. N,N-Diisopropylethylamine (0.107 mL, 0.613 mmol) was then added and the resulting mixture was stirred at 50° C. for 1 h. The reaction mixture was then allowed to cool to RT, and concentrated aqueous hydrochloric acid solution (12 M, 0.286 mL) was added. After 1.5 h, the reaction mixture was diluted with saturated aqueous sodium carbonate solution (20 mL) and ethyl acetate (20 mL). The layers were split and the organic layer was washed with brine (20 mL), was dried over anhydrous sodium sulfate, and was concentrated under reduced pressure. The crude residue was purified via preparatory HPLC (Gemini 5 u C18 100 A 100×30 mm column, 50-100% acetonitrile/water gradient) to afford the product. ¹H NMR (400 MHz, methanol-d₄) δ 7.80 (s, 0.7H), 7.78 (s, 0.3H), 7.36-7.12 (m, 5H), 6.86-6.82 (m, 1H), 6.75-6.71 (m, 1H), 5.52-5.46 (m, 1H), 4.65-4.30 (m, 4H), 4.07-3.82 (m, 3H), 3.19 (s, 0.9H), 3.16 (s, 2.1H), 1.29 (d, J=7.2 Hz, 3H), 1.16 (s, 1.8H), 1.12 (s, 4.2H). ³¹P NMR (162 MHz, methanol-d₄) δ 3.25 (s), 3.16 (s). LCMS: MS m/z=605.50 [M+1], t_{R}=1.35 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.54 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=4.82 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 165. 4-methoxycyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

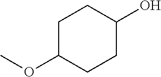

4-methoxycyclohexan-1-ol. The intermediate was obtained from 4-methoxycyclohexanone (1000 mg, 7.80 mmol) in a manner similar to that described for Example 204.

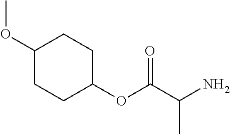

4-methoxycyclohexyl alaninate. The product was prepared from 4-methoxycyclohexanol (700 mg, 5.38 mmol) and cbz-l-alanine (1000 mg, 4.48 mmol) in a manner similar to that described for Intermediate 26.

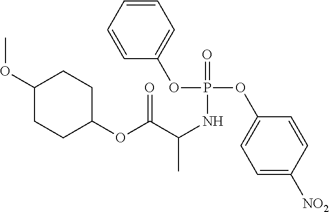 [SMILES: COC1CCC(OC(=O)C(C)NP(=O)(Oc2ccccc2)Oc2ccc([N+](=O)[O-])cc2)CC1]

4-methoxycyclohexyl ((4-nitrophenoxy)(phenoxy) phosphoryl)alaninate. The intermediate was prepared as isomeric mixture from 4-methoxycyclohexyl alaninate (540 mg, 2.68 mmol) in a manner similar to that described for Intermediate 25. ¹H NMR (400 MHz, Chloroform-d) δ 8.26-8.19 (m, 2H), 7.44-7.30 (m, 4H), 7.26-7.17 (m, 3H), 4.92-4.69 (m, 1H), 4.21-4.01 (m, 1H), 3.87 (m, 1H), 3.32 (m, 3H), 3.31-3.18 (m, 1H), 1.92 (m, 2H), 1.83-1.53 (m, 4H), 1.41 (m, 5H). ³¹P NMR (162 MHz, Chloroform-d) δ −3.03, −3.12. MS m/z=479 [M+H].

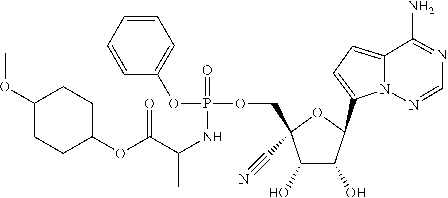 [SMILES: COC1CCC(OC(=O)C(C)NP(=O)(OC[C@@]2(C#N)O[C@@H](c3ccc4c(N)ncnn34)[C@H](O)[C@@H]2O)Oc2ccccc2)CC1]

4-methoxycyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate. The product was obtained from 4-methoxycyclohexyl ((4-nitrophenoxy)(phenoxy) phosphoryl)alaninate (141 mg, 0.29 mmol) and Intermediate 4 (65 mg, 0.20 mmol) in a manner similar to that described for Example 3. ¹H NMR (400 MHz, Methanol-d4) δ 7.85-7.71 (m, 1H), 7.36-7.12 (m, 5H), 6.84 (m, 1H), 6.72 (m, 1H), 5.52 (m, 1H), 4.81-4.55 (m, 2H), 4.55-4.28 (m, 3H), 3.89 (m, 1H), 3.29 (m, 4H), 1.97-1.78 (m, 2H), 1.81-1.47 (m, 4H), 1.49-1.12 (m, 5H). ³¹P NMR (162 MHz, Methanol-d₄) δ 3.45, 3.40, 3.29, 3.28, 3.25, 3.08, 3.04. MS m/z=631 [M+H].

### Example 166. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl cyclohexane carboxylate

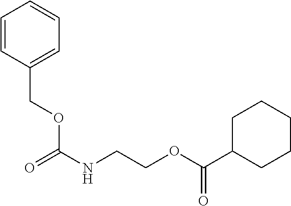 [SMILES: O=C(NCCOC(=O)C1CCCCC1)OCc1ccccc1]

2-(((benzyloxy)carbonyl)amino)ethyl cyclohexanecarboxylate. Cyclohexanecarboxylic acid (256 mg, 2 mmol) was dissolved in anhydrous acetonitrile (6 mL). EDCI (422 mg, 2.2 mmol) was added in one portion, and the reaction was stirred for 15 mins. N-Cbz-aminoethanol (390 mg, 2 mmol) was added in one portion and then DMAP (269 mg, 2.2 mmol) was added. Reaction was stirred for 16 hrs. Reaction was diluted with EtOAc (30 mL) and washed with 5% aqueous citric acid solution (15 mL), followed with saturated aqueous sodium bicarbonate solution (15 mL) and finally with brine (10 mL). Organic extract was dried over anhydrous sodium sulfate and concentrated under reduced pressure. The crude residue was purified via SiO₂ column chromatography (24 g SiO₂ Combiflash HP Gold Column, 0-40% ethyl acetate/hexanes) to afford the intermediate. ¹H NMR (400 MHz, Chloroform-d) δ 7.42-7.28 (m, 5H), 5.11 (s, 2H), 4.96 (s, 1H), 4.15 (t, J=5.2 Hz, 2H), 3.47 (q, J=5.6 Hz, 2H), 2.29 (m, 1H), 1.88 (m, 2H), 1.75 (m, 2H), 1.62 (m, 2H), 1.42 (m, 2H), 1.25 (m, 3H).

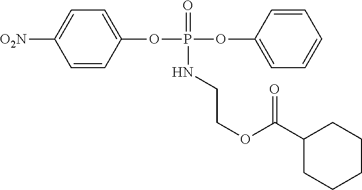 [SMILES: O=C(OCCNP(=O)(Oc1ccccc1)Oc1ccc([N+](=O)[O-])cc1)C1CCCCC1]

2-(((4-nitrophenoxy)(phenoxy)phosphoryl)amino)ethyl cyclohexanecarboxylate. 2-(((benzyloxy)carbonyl)amino)ethyl cyclohexanecarboxylate (372 mg, 1.22 mmol) was dissolved in anhydrous THF (20 mL). 10% Pd/C Degussa type was added, and the mixture was stirred under atmospheric hydrogen for 3 hrs. Catalyst was filtered off and washed with anhydrous THF (5 mL). Filtrate was concentrated under reduced pressure, and the resulting material was used without purification. Phenyl dichlorophosphate (181 uL, 1.22 mmol) was dissolved in anhydrous DCM (10 mL) and stirred under atmospheric nitrogen in an ice bath. Above prepared material was dissolved with anhydrous DCM (2 mL) and added to the reaction dropwise over 5 mins. Reaction was stirred for 1 hr. Triethylamine (374 uL, 2.68 mmol) was added to the reaction mixture dropwise. Reaction was stirred for 1 hr. p-Nitrophenol (136 mg, 0.976 mmol) was added to the reaction in one portion. Ice bath was removed, and the reaction mixture was stirred for 16 hrs. Reaction was diluted with DCM (15 mL) and washed with water (2×20 mL). Dried organic extract over anhydrous sodium sulfate and concentrated under reduced pressure. The crude residue was purified via SiO₂ column chromatography (24 g SiO₂ Combiflash HP Gold Column, 0-30% ethyl acetate/hexanes) to afford the intermediate. ¹H NMR (400 MHz, Chloroform-d) δ 8.29-8.17 (m, 2H), 7.46-7.29 (m, 4H), 7.29-7.13 (m, 3H), 4.19-4.06 (m, 2H), 3.39 (m, 2H), 2.24 (m, 1H), 2.00-1.54 (m, 5H), 1.54-1.11 (m, 5H). ³¹P NMR (162 MHz, Chloroform-d) δ −1.51 (s). MS m/z=449.0 [M+1]; 447.2 [M−1].

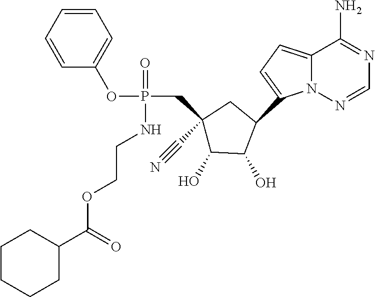 [SMILES: N#C[C@]1(CP(=O)(NCCOC(=O)C2CCCCC2)Oc2ccccc2)C[C@@H](c2ccc3c(N)ncnn23)[C@H](O)[C@@H]1O]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl cyclohexane carboxylate. Intermediate 4 (50 mg, 0.15 mmol) and 2-(((4-nitrophenoxy)(phenoxy)phosphoryl)amino)ethyl cyclohexanecarboxylate (81 mg, 0.18 mmol) were mixed in anhydrous THF (3 mL). Magnesium chloride (57 mg, 0.6 mmol) was added in one portion. Reaction was stirred for 20 mins. DIPEA (52 uL, 0.3 mmol) was added, and the reaction was stirred at 50° C. for 16 hrs. Reaction was cooled to RT, diluted with EtOAc (15 mL) and washed with 2% aqueous sodium carbonate solution (2×10 mL) and followed with brine (10 mL). Organic extract was dried over anhydrous sodium sulfate and concentrated under reduced pressure. Resulting oil was dissolved in MeCN (2 mL) and stirred in an ice bath. 12 M HCl(aq) (300 uL) was added dropwise to the reaction and then stirred for 1 hr. Reaction was diluted with EtOAc (10 mL). Saturated aqueous sodium bicarbonate solution was added to give of pH 8. Organic layer was collected, washed with brine (10 mL), dried over anhydrous sodium sulfate and concentrated under reduced pressure. The crude residue was purified via SiO₂ column chromatography (4 g SiO₂ Combiflash HP Gold Column, 0-3-8% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.79 (m, 1H), 7.31 (m, 2H), 7.25-7.11 (m, 3H), 6.84 (m, 1H), 6.73 (m, 1H), 5.51 (m, 1H), 4.63 (m, 1H), 4.48 (m, 1H), 4.45-4.29 (m, 2H), 3.98 (m, 2H), 3.14 (m, 2H), 2.24 (m, 1H), 1.80 (m, 2H), 1.74-1.49 (m, 3H), 1.45-1.10 (m, 5H). ³¹P NMR (162 MHz, Methanol-d4) δ 5.20 (s), 5.03 (s). MS m/z=601.1 [M+1]; 599.1 [M−1].

### Example 167. (R)-2-(allyloxy)-3-(octadecyloxy)propyl (((2R,3S,4R,5S)-5-(4-aminopyrrolo[2,1-f][1,2,4]triazin-7-yl)-2-cyano-3,4-dihydroxytetrahydrofuran-2-yl)methyl) hydrogen phosphate

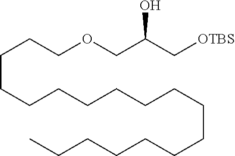 [SMILES: CCCCCCCCCCCCCCCCCCOC[C@@H](O)CO[Si](C)(C)C(C)(C)C]

(R)-1-((tert-butyldimethylsilyl)oxy)-3-(octadecyloxy)propan-2-ol. 1-O-octadecyl-sn-glycerol (3 g, 8.706 mmol) was taken up in pyridine (23 mL), DMF (5 mL) and DCM (5 mL). Imidazole was added the reaction flask was cooled in an ice bath. A solution of TBSCl (1.443 g, 0.01 mol) in pyridine (23 mL) was added in a dropwise fashion via addition funnel. Upon complete addition sitting was continued with the reaction flask in an ice bath. After 1 hour 25 minutes the ice bath was removed and stirring at room temperature was continued for 2 hours 50 minutes. The reaction was cooled in an ice bath and quenched with a 1:1 mixture of water and a saturated aqueous solution of NaHCO₃. The mixture was further diluted with a 1:4 mixture of water and a saturated aqueous solution of NaHCO₃ and the resulting mixture was extracted with hexanes. The combined organic washes were washed with brine and dried over Na₂SO₄. After removal of the drying agent by filtration, the filtrate was concentrated and the intermediate was isolated by silica gel column chromatography (24 g load cartridge, 120 g Combiflash HP Gold Column, eluent ramp from 100% hexanes to 30% EtOAc/hexanes). ¹H NMR (400 MHz, Chloroform-d) δ 3.79 (p, J=5.4 Hz, 1H), 3.67-3.57 (m, 2H), 3.48-3.37 (m, 4H), 1.60-1.49 (m, 2H), 1.34-1.19 (m, 30H), 0.90-0.83 (m, 12H), 0.05 (s, 6H).

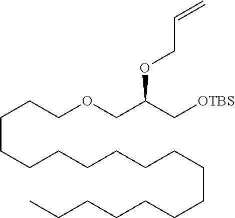 [SMILES: C=CCO[C@H](COCCCCCCCCCCCCCCCCCC)CO[Si](C)(C)C(C)(C)C]

(R)-(2-(allyloxy)-3-(octadecyloxy)propoxy)(tert-butyl)dimethylsilane. NaH, 60% w/w dispersion in mineral oil (0.439 g, 0.011 mol) was suspended in THF (30 mL). The reaction flask was placed in an ice bath and a solution of (R)-1-((tert-butyldimethylsilyl)oxy)-3-(octadecyloxy)propan-2-ol (3.36 g, 7.323 mmol) in THF (15 mL) was added to the reaction in a dropwise fashion via addition funnel. The cold bath was removed and after 30 minutes a solution of allyl bromide (1.267 mL, 0.015 mol) in THF (4 mL) was added in a drop-wise fashion by addition funnel. The reaction was stirred at room temperature for 24 hours. The reaction was cooled in an ice bath and quenched via the addition of a saturated aqueous solution of NH₄Cl. The resulting mixture was diluted with water and hexanes and the layers were separated. The aqueous layer was extracted with hexanes and the combined organics were washed with brine and dried over Na₂SO₄. The drying agent was removed by filtration and the filtrate was concentrated. The intermediate was isolated from the residue by silica gel column chromatography (12 g load cartridge, 120 g Combiflash HP Gold Column, eluent ramp from 100% hexanes to 15% EtOAc/hexanes). ¹H NMR (400 MHz, Chloroform-d) δ 5.90 (ddt, J=17.3, 10.4, 5.6 Hz, 1H), 5.25 (dq, J=17.2, 1.7 Hz, 1H), 5.13 (dq, J=10.4, 1.4 Hz, 1H), 4.13 (dt, J=5.8, 1.5 Hz, 2H), 3.70-3.58 (m, 2H), 3.57-3.48 (m, 2H), 3.47-3.36 (m, 3H), 1.58-1.49 (m, 2H), 1.34-1.19 (s, 30H), 0.90-0.83 (s, 12H), 0.04 (s, 6H).

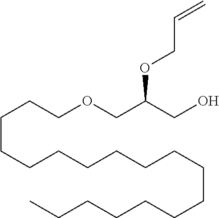 [SMILES: C=CCO[C@@H](CO)COCCCCCCCCCCCCCCCCCC]

(S)-2-(allyloxy)-3-(octadecyloxy)propan-1-ol. (R)-(2-(allyloxy)-3-(octadecyloxy)propoxy)(tert-butyl)dimethylsilane (1.74 g, 3.49 mmol) was dissolved in THF (20 mL). A 1 M solution of TBAF in THF (10.46 mL, 10.46 mmol) was added at room temperature and the reaction was allowed to stir at room temperature for until starting material was consumed, as determined by TLC. The reaction was cooled in an ice bath and quenched via the addition of a saturated aqueous solution of NH₄Cl. The mixture was diluted with DCM and water and the layers were separated. The aqueous phase was extracted with DCM (3×) and the combined organics were washed with brine and dried over Na₂SO₄. The drying agent was removed by vacuum filtration and the filtrate was concentrated. The intermediate was isolated from the residue by silica gel column chromatography (12 g load cartridge, 80 g Combiflash HP Gold Column, eluent ramp from 100% hexanes to 30% EtOAc/hexanes). ¹H NMR (400 MHz, Chloroform-d) δ 5.91 (ddt, J=17.3, 10.4, 5.7 Hz, 1H), 5.27 (dq, J=17.2, 1.6 Hz, 1H), 5.17 (dq, J=10.4, 1.4 Hz, 1H), 4.19-4.04 (m, 2H), 3.76-3.35 (m, 7H), 1.54 (p, J=6.7 Hz, 2H), 1.23 (brs, 30H), 0.86 (t, J=6.8 Hz, 3H).

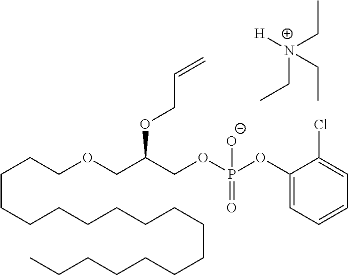

triethylammonium (R)-2-(allyloxy)-3-(octadecyloxy)propyl (2-chlorophenyl) phosphate. The intermediate was prepared in a manner similar to that described for Example 96. ¹H NMR (400 MHz, ACN-d₃) δ 7.63 (dt, J=8.2, 1.2 Hz, 1H), 7.39 (dt, J=8.0, 1.3 Hz, 1H), 7.26-7.21 (m, 1H), 7.02 (td, J=7.7, 1.5 Hz, 1H), 5.90 (ddt, J=17.3, 10.7, 5.5 Hz, 1H), 5.25 (dq, J=17.3, 1.8 Hz, 1H), 5.11 (dq, J=10.5, 1.5 Hz, 1H), 4.07 (dt, J=5.5, 1.5 Hz, 2H), 4.01-3.86 (m, 2H), 3.62 (p, J=5.1 Hz, 1H), 3.49-3.40 (m, 2H), 3.40-3.35 (m, 2H), 3.00 (qd, J=7.3, 4.5 Hz, 6H), 1.50 (p, J=6.8 Hz, 2H), 1.29 (s, 30H), 1.23 (t, J=7.3 Hz, 9H), 0.94-0.86 (m, 3H). ³¹P NMR (162 MHz, ACN-d₃) δ −6.00. MS m/z=575.42 [M+1].

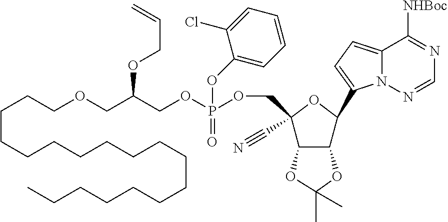 [SMILES: C=CCO[C@H](COCCCCCCCCCCCCCCCCCC)COP(=O)(OC[C@@]1(C#N)O[C@@H](c2ccc3c(NC(=O)OC(C)(C)C)ncnn23)[C@@H]2OC(C)(C)O[C@@H]21)Oc1ccccc1Cl]

tert-butyl (7-((3aS,4S,6R,6aS)-6-(((((R)-2-(allyloxy)-3-(octadecyloxy)propoxy)(2-chlorophenoxy)phosphoryl)oxy)methyl)-6-cyano-2,2-dimethyltetrahydrofuro[3,4-d][1,3]dioxol-4-yl)pyrrolo[2,1-f][1,2,4]triazin-4-yl)carbamate. The intermediate was prepared in a manner similar to that described for Example 96. ¹H NMR (400 MHz, ACN-d₃) δ 8.38 (s, 1H), 8.14 (s, 1H), 7.50-7.43 (m, 1H), 7.42-7.35 (m, 1H), 7.28-7.15 (m, 2H), 7.11 (s, 1H), 6.93 (s, 1H), 5.93-5.79 (m, 1H), 5.72 (s, 1H), 5.34-5.19 (m, 2H), 5.11 (dt, J=6.6, 3.0 Hz, 2H), 4.57-4.45 (m, 2H), 4.34-4.13 (m, 2H), 4.04 (ddt, J=12.7, 5.5, 1.5 Hz, 2H), 3.65 (q, J=5.1 Hz, 1H), 3.45-3.33 (m, 4H), 1.72 (s, 3H), 1.55 (s, 9H), 1.50-1.48 (m, 2H), 1.38 (s, 3H), 1.34-1.24 (m, 30H), 0.94-0.86 (m, 3H). ³¹P NMR (162 MHz, ACN-d₃) δ −7.345 (s), −7.414 (s). MS m/z=989.06 [M+1].

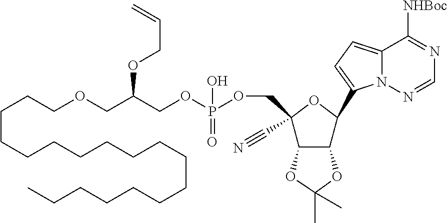 [SMILES: C=CCO[C@H](COCCCCCCCCCCCCCCCCCC)COP(=O)(O)OC[C@@]1(C#N)O[C@@H](c2ccc3c(NC(=O)OC(C)(C)C)ncnn23)[C@@H]2OC(C)(C)O[C@@H]21]

tert-butyl (7-((3aS,4S,6R,6aS)-6-(((((R)-2-(allyloxy)-3-(octadecyloxy)propoxy)(hydroxy)phosphoryl)oxy)methyl)-6-cyano-2,2-dimethyltetrahydrofuro[3,4-d][1,3]dioxol-4-yl)pyrrolo[2,1-f][1,2,4]triazin-4-yl)carbamate. Tert-butyl (7-((3aS,4S,6R,6aS)-6-(((((R)-2-(allyloxy)-3-(octadecyloxy)propoxy)(2-chlorophenoxy)phosphoryl)oxy)methyl)-6-cyano-2,2-dimethyltetrahydrofuro [3,4-d][1,3]dioxol-4-yl)pyrrolo[2,1-f][1,2,4]triazin-4-yl)carbamate (0.1 g, 0.101 mmol) was dissolved in THF (1.2 mL). The resulting solution was cooled in an ice bath and a 1 N solution of NaOH in water (0.121 mL, 0.003 mol) was added in a drop-wise manner. The reaction was stirred at room temperature for 55 minutes and then placed in a preheated 45° C. oil bath. After 1 hour 55 minutes additional THF (0.5 mL) was added followed by more of a 1 N solution of NaOH in water (0.2 mL, 0.006 mol). After 2 hours the reaction was cooled to room temperature and stored in a 5° C. refrigerator overnight. The reaction was placed back in a preheated 45° C. oil bath. After 1 hour the reaction was cooled in an ice bath and quenched via the addition of a 2 N solution of HCl in water (0.2 mL). The resulting mixture was diluted with water (10 mL) and DCM (10 mL). The layers were separated and the aqueous layer was extracted with DCM (3×). The combined organics were extracted with brine and dried over Na₂SO₄. The drying agent was removed by filtration and the filtrate was concentrated. The intermediate was isolated from the residue by silica gel column chromatography (12 g load cartridge, 25 g Combiflash HP Gold Column, eluent ramp from 100% DCM to 20% MeOH/DCM, pausing the ramp at 10% MeOH/DCM). MS m/z=878.43 [M+1]

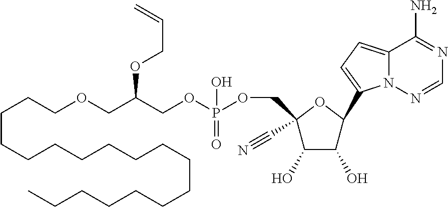 [SMILES: C=CCO[C@H](COCCCCCCCCCCCCCCCCCC)COP(=O)(O)OC[C@@]1(C#N)O[C@@H](c2ccc3c(N)ncnn23)[C@H](O)[C@@H]1O]

(R)-2-(allyloxy)-3-(octadecyloxy)propyl (((2R,3S,4R,5S)-5-(4-aminopyrrolo[2,1-f][1,2,4]triazin-7-yl)-2-cyano-3,4-dihydroxytetrahydrofuran-2-yl)methyl) hydrogen phosphate. The product was prepared in a manner similar to that described for Example 96. ¹H NMR (400 MHz, Methanol-d₄) δ 8.01 (s, 1H), 7.35 (d, J=4.7 Hz, 1H), 7.05 (d, J=4.8 Hz, 1H), 5.90 (ddt, J=17.3, 10.8, 5.6 Hz, 1H), 5.56 (d, J=4.1 Hz, 1H), 5.27 (dq, J=17.3, 1.7 Hz, 1H), 5.11 (dq, J=10.5, 1.5 Hz, 1H), 4.51-4.47 (m, 2H), 4.21 (ddd, J=29.1, 11.0, 5.6 Hz, 2H), 4.12 (dq, J=5.4, 1.7 Hz, 2H), 3.95 (dq, J=13.6, 5.4 Hz, 2H), 3.69 (p, J=5.1 Hz, 1H), 3.58-3.40 (m, 4H), 1.54 (p, J=6.8 Hz, 2H), 1.38-1.23 (m, 30H), 0.94-0.86 (m, 3H). ³¹P NMR (162 MHz, Methanol-d₄) δ −0.417 (s). MS m/z=738.43 [M+1].

### Example 168. cyclohexyl ((S)-(((3aS,4R,6S,6aS)-6-(4-aminopyrrolo[2,1-f][1,2,4]triazin-7-yl)-4-cyano-2-oxotetrahydrofuro[3,4-d][1,3]dioxol-4-yl)methoxy)(phenoxy)phosphoryl)-L-alaninate

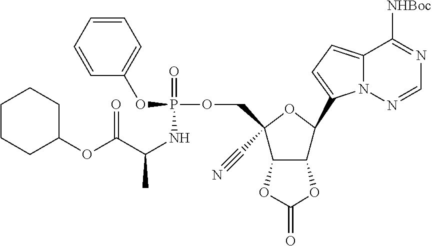 [SMILES: C[C@H](N[P@](=O)(OC[C@@]1(C#N)O[C@@H](c2ccc3c(NC(=O)OC(C)(C)C)ncnn23)[C@@H]2OC(=O)O[C@@H]21)Oc1ccccc1)C(=O)OC1CCCCC1]

Example 6 (10 mg, 0.0167 mmol) was dissolved in anhydrous DMF (1 mL). N, N′-Carbonyldiimidazole (13.5 mg, 0.084 mmol) was added to the reaction. DMAP (2 mg, 0.0167 mmol) was added and the reaction was stirred for 16 hrs.

More N, N′-Carbonyldiimidazole (7.5 mg, 0.045 mmol) was added and the reaction was stirred for 3 hrs. The crude residue was purified via SiO₂ column chromatography (4 g SiO₂ Combiflash HP Gold Column, 0-100% ethyl acetate/hexanes). Fractions having the desired product were combined and concentrated under reduced pressure as oil which was then dissolved in MeCN and water and freeze-dried to afford the product. ¹H NMR (400 MHz, Chloroform-d) δ 7.88 (s, 1H), 7.35 (t, J=7.8 Hz, 2H), 7.28-7.14 (m, 3H), 6.75 (d, J=4.5 Hz, 1H), 6.64 (d, J=4.5 Hz, 1H), 6.03 (s, 2H), 5.65 (m, 2H), 5.49 (dt, J=6.7, 1.9 Hz, 1H), 4.76 (td, J=8.9, 4.3 Hz, 1H), 4.48-4.33 (m, 2H), 4.18-3.94 (m, 2H), 2.02-1.61 (m, 5H), 1.58-1.27 (m, 8H). ³¹P NMR (162 MHz, Chloroform-d) δ 2.63 (s). MS m/z=627.0 [M+1]; 625.2 [M−1].

### Example 169. 2-ethylbutyl ((4aR,6S,7S,7aS)-6-(4-aminopyrrolo[2,1-f][1,2,4]triazin-7-yl)-4a-cyano-7-hydroxy-2-oxidotetrahydro-4H-furo[3,2-d][1,3,2]dioxaphosphinin-2-yl)-L-alaninate

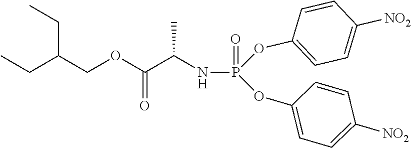 [SMILES: CCC(CC)COC(=O)[C@H](C)NP(=O)(Oc1ccc([N+](=O)[O-])cc1)Oc1ccc([N+](=O)[O-])cc1]

2-ethylbutyl (bis(4-nitrophenoxy)phosphoryl)-L-alaninate. To a solution of 4-nitrophenol (0.52 g, 3.74 mmol) and 4-nitrophenyl phosphorodichloridate (1.06 g, 4.14 mmol) in dichloromethane (15 mL) was added triethylamine (0.61 mL, 4.38 mmol) at −40° C. under argon atmosphere. The resulting mixture was allowed to warm to RT and stirred for 0.5 h. The 2-ethylbutyl L-alaninate hydrochloride (0.85 g, 4.05 mmol) and triethylamine (1.22 mL, 8.77 mmol) were then added. After 1 h, the reaction mixture was diluted with hexane (15 mL) and the resulting mixture was filtered. The filtrate was purified by silica gel column eluted with 20-33% ethyl acetate/hexanes to afford the intermediate. ¹H NMR (400 MHz, CDCl₃) δ 8.25 (d, J=9.2 Hz, 4H), 7.2-7.4 (m, 4H), 4.0-4.2 (m, 3H), 1.48-1.58 (m, 1H), 1.43 (d, J=7.2 Hz, 3H), 1.3-1.4 (m, 4H), 0.87 (t, J=7.2 Hz, 6H). ³¹P NMR (162 MHz, CDCl₃) δ −3.47 (s).

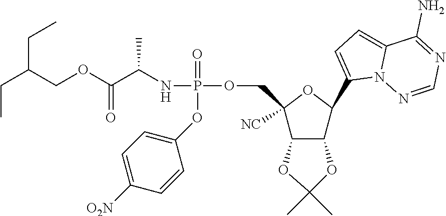 [SMILES: CCC(CC)COC(=O)[C@H](C)NP(=O)(OC[C@@]1(C#N)O[C@@H](c2ccc3c(N)ncnn23)[C@@H]2OC(C)(C)O[C@@H]21)Oc1ccc([N+](=O)[O-])cc1]

2-ethylbutyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(4-nitrophenoxy)phosphoryl)-L-alaninate. To a mixture of Intermediate 4 (155 mg, 0.468 mmol), 2-ethylbutyl (bis(4-nitrophenoxy)phosphoryl)-L-alaninate (255 mg, 0.515 mmol), and magnesium chloride (85 mg, 0.893 mmol) in THF (2.0 mL) was added N,N-Diisopropylethylamine (200 μL, 0.115 mmol). The resulting mixture was stirred at RT for 16 h. The reaction mixture was diluted with ethyl acetate (5 mL), washed with water, dried over MgSO₄ and concentrated under reduced pressure. The crude residue was purified by silica gel column eluted with 30-100% ethyl acetate/hexane to afford the intermediate. ¹H NMR (400 MHz, CDCl3) δ 7.9-8.19 (m, 3H), 7.2-7.4 (m, 2H), 6.66-6.73 (m, 2H), 5.62 (brs, 1H), 5.24-5.3 (m, 1H), 5.0-5.1 (m, 1H), 4.3-4.55 (m, 2H). 3.7-4.2 (m, 3H), 2.0-2.1 (2s, 3H), 1.75-1.78 (2s, 3H), 1.2-1.55 (m, 8H), 0.87 (t J=7.2 Hz, 3H). ³¹P NMR (162 MHz, CDCl₃) δ 1.92 (s), 1.66 (s). MS m/z=688.2 [M+H].

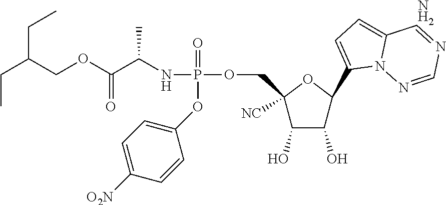

2-ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(4-nitrophenoxy)phosphoryl)-L-alaninate. To a solution of 2-ethylbutyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(4-nitrophenoxy)phosphoryl)-L-alaninate (105 mg, 0.153 mmol) in acetonitrile (1.2 mL) and water (0.2 mL) was added HCl (0.15 mL, 37%) at 0° C. The reaction mixture was stirred at RT for 3 h. The reaction mixture was quenched with solid NaHCO₃ (210 mg), stirred for 1 h and concentrated. The residue was treated with ethyl acetate, washed with water, dried over anhydrous sodium sulfate, filtered, concentrated and dried in high vacuum to afford the intermediate. ¹H NMR (400 MHz, CDCl₃+5% CD₃OD) δ 8.04-8.18 (m, 2H), 7.74-7.76 (m, 1H), 7.02-7.32 (m, 2H), 6.60-6.65 (m, 2H), 5.48 (d, J=3.6 Hz, 1H), 4.3-4.6 (m, 3H), 3.8-4.1 (m, 3H), 1.2-1.5 (m, 8H), 0.7-0.9 (m, 6H). ³¹P NMR (162 MHz, CDCl₃+5% CD₃OD) δ 2.39 (s), 2.33 (s). MS m/z=648.1 [M+H].

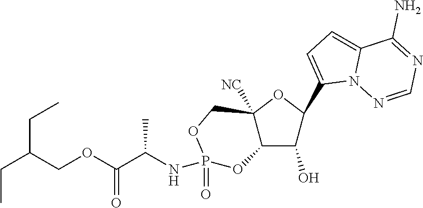

2-ethylbutyl ((4aR,6S,7S,7aS)-6-(4-aminopyrrolo[2,1-f][1,2,4]triazin-7-yl)-4a-cyano-7-hydroxy-2-oxidotetrahydro-4H-furo[3,2-d][1,3,2]dioxaphosphinin-2-yl)-L-alaninate. The mixture of 2-ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(4-nitrophenoxy)phosphoryl)-L-alaninate (82 mg, 0.127 mmol) and DMAP (164 mg, 1.342 mmol) in ethyl acetate (10 mL) was stirred at 55° C. for 16 h. The reaction mixture was concentrated under reduced pressure. The crude residue was purified by silica gel column eluted with 50-100% ethyl acetate/hexane to afford the product. ¹H NMR (400 MHz, CD₃OD) δ 7.75 (s, 1H), 6.76 (d, J=4.8 Hz, 1H) 6.66 (d, J=4.8 Hz, 1H), 5.52 (d, J=1.2 Hz, 1H), 5.26 (dd, J=4.8, 3.2 Hz, 1H), 4.68 (d, J=4.8 Hz, 1H), 4.48-4.59 (m, 2H), 4.05-4.11 (m, 1H), 4.0 (d, J=5.6 Hz, 2H), 1.4-1.47 (m, 1H), 1.28-1.37 (m, 7H), 0.83 (t, J=7.6 Hz, 3H). ³¹P NMR (162 MHz, CD₃OD) δ 6.9 (s). MS m/z=509.1 [M+H].

### Example 170. 2-ethylbutyl ((4aR,6S,7S,7aS)-6-(4-aminopyrrolo[2,1-f][1,2,4]triazin-7-yl)-4a-cyano-7-hydroxy-2-oxidotetrahydro-4H-furo[3,2-d][1,3,2]dioxaphosphinin-2-yl)-L-alaninate

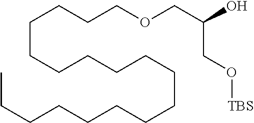

(R)-1-((tert-butyldimethylsilyl)oxy)-3-(octadecyloxy)propan-2-ol. 1-O-octadecyl-2-O-benzyl-sn-glyerol (869 mg, 2 mmol) was dissolved in THF (50 mL). Degussa type 10% palladium on activated carbon (50 mg) was added to the reaction solution which was then stirred under atmospheric hydrogen for 2 hrs. Catalyst was filtered off through Celite. Filtrate was concentrated under reduced pressure. Resulting solid was dissolved in anhydrous THF (20 mL) and stirred at RT. Imidazole (207 mg, 3 mmol) and tert-Butyldimethylsilyl chloride (275 mg, 1.8 mmol) were added to the reaction which was then stirred for 4 hrs. More imidazole (207 mg, 3 mmol) and TBS-Cl (275 mg, 1.8 mmol) were added and the reaction was stirred for 16 hrs. More imidazole (207 mg, 3 mmol) and TBS-Cl (140 mg, 0.9 mmol) were added and the reaction was stirred for 3 hrs.

Reaction was diluted with EtOAc (40 mL) and washed with saturated sodium bicarbonate solution (10 mL) and then with brine (10 mL). Organic layer was dried over anhydrous sodium sulfate and concentrated under reduced pressure. The crude residue was purified via SiO₂ column chromatography (24 g SiO₂ Combiflash HP Gold Column, 0-5% ethyl acetate/hexanes). Fractions having the desired product were combined and concentrated under reduced pressure to afford the intermediate. ¹H NMR (400 MHz, Chloroform-d) δ 3.80 (p, J=5.4 Hz, 1H), 3.70-3.58 (m, 2H), 3.50-3.38 (m, 4H), 1.56 (q, J=6.9 Hz, 2H), 1.25 (m, 30H), 0.94-0.81 (m, 12H), 0.07 (s, 6H).

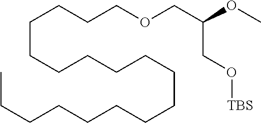

(R)-tert-butyl(2-methoxy-3-(octadecyloxy)propoxy)dimethylsilane. Sodium hydride (60% in oil) (57 mg, 1.43 mmol) was suspended in anhydrous THF (5 mL) and stirred in an ice bath under atmospheric nitrogen. (R)-1-((tert-butyldimethylsilyl)oxy)-3-(octadecyloxy)propan-2-ol (437 mg, 0.95 mmol) was dissolved in anhydrous THF (5 mL) and added to the suspension dropwise. Resulting reaction mixture was stirred for 30 mins. Iodomethane (89 uL, 1.43 mmol) was added dropwise. Ice bath was removed and the reaction was stirred for 2 hrs.

Reaction was diluted with EtOAc (20 mL) and saturated aqueous sodium bicarbonate (10 mL) was added slowly. Organic layer was collected, washed with saturated aqueous sodium bicarbonate (10 mL) followed with brine (10 mL), dried over anhydrous sodium sulfate and concentrated under reduced pressure. The crude residue was purified via SiO₂ column chromatography (12 g SiO₂ Combiflash HP Gold Column, 0-5% ethyl acetate/hexanes). Fractions having the desired product were combined and concentrated under reduced pressure to afford the intermediate. ¹H NMR (400 MHz, Chloroform-d) δ 3.72-3.60 (m, 2H), 3.53 (m, 1H), 3.49-3.31 (m, 7H), 1.56 (m, 2H), 1.25 (m, 30H), 0.94-0.80 (m, 12H), 0.06 (s, 6H).

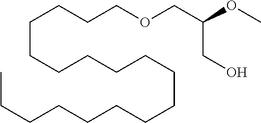 [SMILES: CCCCCCCCCCCCCCCCCCOC[C@H](CO)OC]

(S)-2-methoxy-3-(octadecyloxy)propan-1-ol. (R)-tert-butyl(2-methoxy-3-(octadecyloxy)propoxy)dimethylsilane (381 mg, 0.806 mmol) was dissolved in anhydrous THF (5 mL). TBAF trihydrate (381 mg, 1.21 mmol) was added in one portion. Reaction was stirred for 3 hrs. Reaction was diluted with EtOAc (20 mL) and washed with saturated sodium bicarbonate solution (10 mL) followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions having the desired product were combined and concentrated under reduced pressure to afford the intermediate. ¹H NMR (400 MHz, Chloroform-d) δ 3.76-3.65 (m, 2H), 3.54 (m, 2H), 3.49-3.38 (m, 6H), 1.57 (m, 2H), 1.25 (m, 30H), 0.88 (t, J=6.7 Hz, 3H).

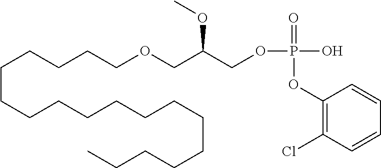 [SMILES: CCCCCCCCCCCCCCCCCCOC[C@H](COP(=O)(O)Oc1ccccc1Cl)OC]

2-chlorophenyl ((R)-2-methoxy-3-(octadecyloxy)propyl) hydrogen phosphate. 2-Chlorophenyl dichlorophosphate (145 uL, 0.897 mmol) was dissolved in anhydrous acetonitrile (4 mL) and stirred in an ice bath under atmospheric nitrogen. 1,2,4 triazole (103 mg, 1.494 mmol) was added in one portion. Triethylamine (208 uL, 1.494 mmol) was added in one portion. (S)-2-methoxy-3-(octadecyloxy)propan-1-ol (268 mg, 0.747 mmol) was dissolved in pyridine (4 mL) and added to the reaction mixture dropwise. Ice bath was removed and the reaction was stirred for 4 hrs. More 1,2,4 triazole (50 mg, 0.75 mmol) and triethylamine (50 uL, 0.37 mmol) were added and the reaction was stirred for 2 hrs.

Water (500 uL) and triethylamine (1 mL) were added to the reaction and then stirred for 20 mins. Reaction was then diluted with EtOAc (20 mL) and washed with saturated aqueous sodium bicarbonate solution (2×10 mL) followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-20% methanol/DCM). Fractions having the desired product were combined and concentrated under reduced pressure to afford the intermediate. ¹H NMR (400 MHz, Chloroform-d) δ 7.68 (d, J=8.2 Hz, 1H), 7.23 (d, J=8.0 Hz, 1H), 7.11-7.01 (m, 1H), 6.87 (t, J=7.7 Hz, 1H), 3.98 (d, J=5.9 Hz, 2H), 3.29 (m, 6H), 3.20 (m, 2H), 3.04 (qd, J=7.3, 4.5 Hz, 1H), 1.42 (t, J=6.9 Hz, 2H), 1.24 (m, 30H), 0.88 (t, J=6.7 Hz, 3H). ³¹P NMR (162 MHz, Chloroform-d) δ −3.91 (s).

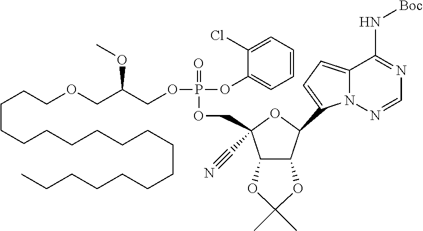 [SMILES: CCCCCCCCCCCCCCCCCCOC[C@H](COP(=O)(OC[C@@]1(C#N)O[C@@H](c2ccc3c(NC(=O)OC(C)(C)C)ncnn23)[C@@H]2OC(C)(C)O[C@@H]21)Oc1ccccc1Cl)OC]

tert-butyl (7-((3aS,4S,6R,6aS)-6-((((2-chlorophenoxy)((R)-2-methoxy-3-(octadecyloxy) propoxy)phosphoryl)oxy)methyl)-6-cyano-2,2-dimethyltetrahydrofuro[3,4-d][1,3]dioxol-4-yl)pyrrolo[2,1-f][1,2,4]triazin-4-yl)carbamate. 2-chlorophenyl ((R)-2-methoxy-3-(octadecyloxy)propyl) hydrogen phosphate (290 mg, 0.528 mmol) was dissolved in anhydrous pyridine (5 mL). 1-(Mesitylene-2-sulfonyl)-3-nitro-1,2,4-triazole (284 mg, 0.96 mmol) was added in one portion. Intermediate 2 (206 mg, 0.48 mmol) was added in one portion and stirred for 30 mins. 1-methyl-imidazole (77 uL, 0.96 mmol) was added in one portion and the reaction mixture was stirred for 2 hrs. More 1-(Mesitylene-2-sulfonyl)-3-nitro-1,2,4-triazole (284 mg, 0.96 mmol) was added and stirred for 20 mins. 1-methyl-imidazole (100 uL) was added and the reaction was stirred for 48 hrs.

Reaction was diluted with EtOAc (25 mL) and washed with saturated sodium bicarbonate solution (3×10 mL), 5% aqueous citric acid solution (2×10 mL) and then with brine (10 mL). Organic layer was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions having the desired product were combined and concentrated under reduced pressure to afford the intermediate. ¹H NMR (400 MHz, Chloroform-d) δ 8.01 (m, 1H), 7.48-7.31 (m, 2H), 7.24-7.03 (m, 3H), 6.92-6.76 (m, 2H), 5.70 (m, 1H), 5.21 (m, 1H), 5.16-5.00 (m, 1H), 4.60-4.17 (m, 4H), 3.63-3.30 (m, 7H), 1.76 (d, J=7.1 Hz, 2H), 1.57 (m, 12H), 1.37 (m, 3H), 1.25 (m, 30H), 0.88 (t, J=6.7 Hz, 3H). ³¹P NMR (162 MHz, Chloroform-d) δ −7.42 (s), −7.45 (s).

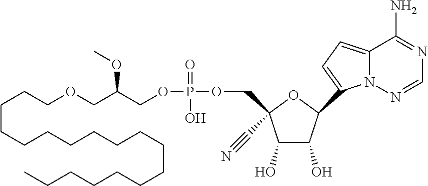 [SMILES: CCCCCCCCCCCCCCCCCCOC[C@H](COP(=O)(O)OC[C@@]1(C#N)O[C@@H](c2ccc3c(N)ncnn23)[C@H](O)[C@@H]1O)OC]

((2R,3S,4R,5S)-5-(4-aminopyrrolo[2,1-f][1,2,4]triazin-7-yl)-2-cyano-3,4-dihydroxytetrahydrofuran-2-yl)methyl ((R)-2-methoxy-3-(octadecyloxy)propyl) hydrogen phosphate. tert-butyl (7-((3aS,4S,6R,6aS)-6-((((2-chlorophenoxy)((R)-2-methoxy-3-(octadecyloxy) propoxy)phosphoryl)oxy)methyl)-6-cyano-2,2-dimethyltetrahydrofuro[3,4-d][1,3]dioxol-4-yl)pyrrolo[2,1-f][1,2,4]triazin-4-yl)carbamate (360 mg, 0.274 mmol) was dissolved in acetonitrile (5 mL). 1 N NaOH(aq) (500 uL) was added and the reaction was stirred for 18 hrs. 1 N NaOH(aq) (500 uL) was added and the reaction was stirred at 30° C. for 6 hrs. More 1 N NaOH(aq) (500 uL) was added and the reaction was stirred at 30° C. for 3 hrs and then at RT for 16 hrs. Reaction was diluted with EtOAc (25 mL) and washed with saturated sodium bicarbonate solution (3×10 mL) and then with brine (10 mL). Organic layer was dried over anhydrous sodium sulfate and concentrated under reduced pressure. The crude residue was purified via SiO₂ column chromatography (12 g SiO₂ Combiflash HP Gold Column, 0-30% methanol/DCM). Fractions having the desired product were combined and concentrated under reduced pressure and the resulting product was then dissolved in 7 mL of 70% TFA in water and stirred at RT. Reaction was monitored with HPLC and LC-MS. After 5 hrs, reaction was cooled in an ice bath. 1 N NaOH(aq) was slowly added to give pH of 4. Precipitate was formed and was collected by filtration. Solid was dissolved in 1 M triethylammonium bicarbonate solution and acetonitrile. The crude residue was purified via C18 column (20-100% MeCN with buffer A being 0.1 M triethylammonium bicarbonate). Fractions having the desired product were combined and freeze-dried to afford the product as a triethylammonium salt. ¹H NMR (400 MHz, Methanol-d4) δ 7.87 (s, 1H), 7.02 (d, J=4.6 Hz, 1H), 6.91 (d, J=4.6 Hz, 1H), 5.54 (d, J=5.3 Hz, 1H), 4.55 (t, J=5.4 Hz, 1H), 4.50 (d, J=5.4 Hz, 1H), 4.23-4.07 (m, 2H), 3.97-3.82 (m, 2H), 3.54-3.46 (m, 2H), 3.46-3.36 (m, 6H), 3.20 (q, J=7.3 Hz, 4H), 1.51 (m, 2H), 1.36-1.20 (m, 36H), 0.89 (t, J=6.7 Hz, 3H). ³¹P NMR (162 MHz, Methanol-d4) δ −0.38 (s). MS m/z=712.2 [M+1]; 710.5 [M−1].

### Example 171. cyclooctyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

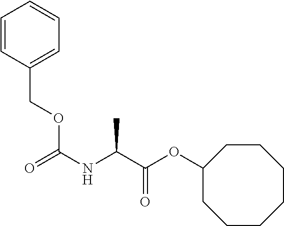 [SMILES: C[C@H](NC(=O)OCc1ccccc1)C(=O)OC1CCCCCCC1]

cyclooctyl ((benzyloxy)carbonyl)-L-alaninate. Cbz-L-Ala (446 mg, 2 mmol) was dissolved in anhydrous MeCN (15 mL). EDCI (422 mg, 2.2 mmol) was added in one portion and the reaction was stirred for 15 mins. Cyclooctanol (291 uL, 2.2 mmol) was added. DMAP (269 mg, 2.2 mmol) was then added in one portion. Reaction was stirred for 16 hrs.

Reaction was diluted reaction with EtOAc (30 mL) and washed with 5% aqueous citric acid solution (10 mL), followed with saturated aqueous sodium bicarbonate solution (10 mL) and finally with brine (10 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 7.41-7.28 (m, 5H), 5.31 (d, J=7.5 Hz, 1H), 5.11 (s, 2H), 4.97 (td, J=8.3, 4.2 Hz, 1H), 4.32 (t, J=7.4 Hz, 1H), 1.88-1.43 (m, 14H), 1.39 (d, J=7.1 Hz, 3H).

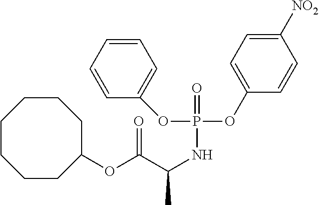 [SMILES: C[C@H](NP(=O)(Oc1ccccc1)Oc1ccc([N+](=O)[O-])cc1)C(=O)OC1CCCCCCC1]

cyclooctyl ((4-nitrophenoxy)(phenoxy)phosphoryl)-L-alaninate. Cyclooctyl ((benzyloxy)carbonyl)-L-alaninate (440 mg, 1.34 mmol) was dissolved in anhydrous THF (15 mL). 10% Pd/C Degussa type was added and the reaction mixture was stirred under atmospheric hydrogen for 5 hrs. Catalyst was filtered and the filtrate was used without purification.

Phenyl dichlorophosphate (219 uL, 1.47 mmol) was dissolved in anhydrous DCM (10 mL) and stirred in an ice bath under atmospheric nitrogen. Above THF solution was added to the reaction dropwise and then stirred for 20 mins. Triethylamine (448 uL, 3.2 mmol) was added dropwise and then stirred for 30 mins. p-Nitrophenol (168 mg, 1.21 mmol) and triethylamine (224 uL, 1.61 mmol) were added. Ice bath was removed and the reaction mixture was stirred for 14 hrs at RT.

Reaction was diluted with EtOAc (30 mL) and washed with 0.2 M sodium carbonate solution (2×10 mL) and followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 8.29-8.15 (m, 2H), 7.37 (m, 4H), 7.29-7.13 (m, 3H), 4.94 (m, 1H), 4.17-4.01 (m, 1H), 3.89 (m, 1H), 1.83-1.42 (m, 14H), 1.38 (m, 3H). ³¹P NMR (162 MHz, Chloroform-d) δ −2.97 (s), −3.04 (s). MS m/z=476.7 [M+1]; 475.1 [M−1].

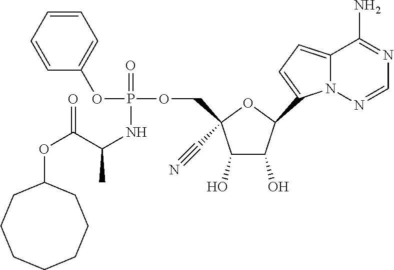 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCCCCCC1]

cyclooctyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.15 mmol) and cyclooctyl ((4-nitrophenoxy)(phenoxy)phosphoryl)-L-alaninate (86 mg, 0.18 mmol) were mixed and dissolved in anhydrous THF (5 mL). Magnesium chloride (86 mg, 0.906 mmol) was added in one portion and the reaction was stirred at 50° C. for 10 mins. DIPEA (158 uL, 0.906 mmol) was added and the reaction was stirred at 50° C. for 2 hrs. More magnesium chloride (50 mg) was added and stirred for 2 hrs. Reaction was stirred at 35° C. for 16 hrs.

Reaction was diluted with EtOAc (20 mL) and washed with 5% citric acid solution (10 mL), with 0.2 M aqueous sodium carbonate solution (2×10 mL) and with brine (10 mL). Organic layer was dried over anhydrous sodium sulfate and concentrated under reduced pressure which was then dissolved in MeCN (5 mL). 12 M HCl(aq) (300 uL) was added dropwise. Reaction was stirred for 1 hr. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions having the desired product were combined and concentrated under reduced pressure and the resulting product was then dissolved in MeCN and water and freeze-dried to afford the product. ¹H NMR (400 MHz, Methanol-d4) δ 7.79 (m, 1H), 7.31 (m, 2H), 7.26-7.10 (m, 3H), 6.84 (m, 1H), 6.73 (m, 1H), 5.50 (m, 2H), 4.88 (m, 1H), 4.62 (m, 1H), 4.54-4.29 (m, 3H), 3.92-3.76 (m, 1H), 1.80-1.35 (m, 14H), 1.24 (d, J=7.1 Hz, 3H). ³¹P NMR (162 MHz, Methanol-d4) δ 3.29 (s). MS m/z=629.0 [M+1]; 627.0 [M−1].

Separation of the (S) and (R) Diastereomers. The product was purified via chiral preparatory HPLC (Chiralpak IA, 150×4.6 mm, SFC-30% Ethanol isocratic) to afford the diastereomers:

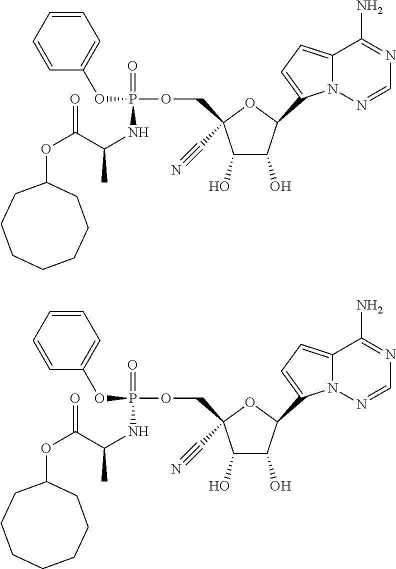

### Example 172. First Eluting Diastereomer

¹H NMR (400 MHz, Methanol-d4) δ 7.78 (s, 1H), 7.37-7.22 (m, 2H), 7.20-7.09 (m, 3H), 6.85 (d, J=4.5 Hz, 1H), 6.73 (d, J=4.5 Hz, 1H), 5.57-5.44 (m, 1H), 4.62 (t, J=5.3 Hz, 1H), 4.54-4.41 (m, 2H), 4.36 (m, 1H), 3.83 (m, 1H), 1.81-1.39 (m, 14H), 1.24 (d, J=7.1, 3H). ³¹P NMR (162 MHz, Methanol-d4) δ 3.30 (s).

### Example 173. Second Eluting Diastereomer

¹H NMR (400 MHz, Methanol-d4) δ 7.80 (s, 1H), 7.33 (m, 2H), 7.27-7.10 (m, 3H), 6.84 (d, J=4.5 Hz, 1H), 6.73 (d, J=4.5 Hz, 1H), 5.50 (d, J=5.0 Hz, 1H), 4.61 (t, J=5.3 Hz, 1H), 4.49-4.26 (m, 3H), 3.85 (dq, J=9.7, 7.0 Hz, 1H), 1.77-1.36 (m, 14H), 1.24 (d, J=7.1 Hz, 3H). ³¹P NMR (162 MHz, Methanol-d4) δ 3.29 (s).

### Example 174. 4,4-difluorocyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

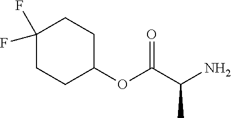

4,4-difluorocyclohexyl L-alaninate. The intermediate was prepared from Cbz-L-alanine (1.1 g, 4.93 mmol) and 4,4-difluorocyclohexanol (1.01 g, 7.39 mmol) in a manner similar to that described for Intermediate 26. MS m/z=208 [M+H].

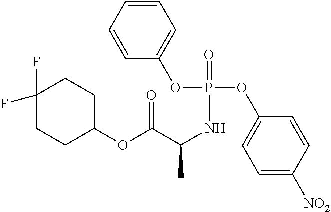

4,4-difluorocyclohexyl ((4-nitrophenoxy)(phenoxy)phosphoryl)-L-alaninate. The intermediate was prepared as isomeric mixture from 4,4-difluorocyclohexyl L-alaninate (673 mg, 3.25 mmol) in a manner similar to that described for Intermediate 25. ¹H NMR (400 MHz, Chloroform-d) δ 8.32-8.19 (m, 2H), 7.43-7.31 (m, 4H), 7.29-7.12 (m, 3H), 4.95 (d, J=7.7 Hz, 1H), 4.23-4.07 (m, 1H), 3.94-3.77 (m, 1H), 2.15-1.69 (m, 8H), 1.41 (ddd, J=7.1, 3.4, 0.7 Hz, 3H). ³¹P NMR (162 MHz, Chloroform-d) δ −3.08, −3.16. ¹⁹F NMR (377 MHz, Chloroform-d) δ −95.69 (d, J=239.1 Hz), −101.23 (d, J=236.5 Hz). MS m/z=485 [M+H].

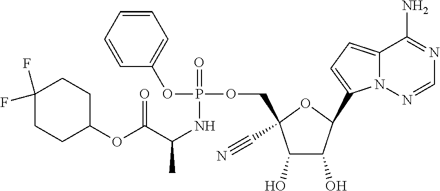 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCC(F)(F)CC1]

4,4-difluorocyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was obtained from 4,4-difluorocyclohexyl ((4-nitrophenoxy)(phenoxy)phosphoryl)-L-alaninate (158 mg, 0.33 mmol) and Intermediate 4 (72 mg, 0.22 mmol) in a manner similar to that described for Example 3. ¹H NMR (400 MHz, Methanol-d4) δ 7.79 (m, 1H), 7.39-7.11 (m, 5H), 6.84 (m, 1H), 6.74 (m, 1H), 5.49 (m, 1H), 4.93-4.80 (m, 1H), 4.62 (m, 1H), 4.54-4.30 (m, 3H), 4.00-3.81 (m, 1H), 2.15-1.63 (m, 8H), 1.27 (m, 3H). ³¹P NMR (162 MHz, Methanol-d4) δ 3.26, 3.24. ¹⁹F NMR (377 MHz, Methanol-d4) δ −96.36 (d, J=233.1 Hz), −102.62 (d, J=236.8 Hz). MS m/z=637 [M+H].

The mixture was separated by SFC (AD-H 21×250 mm column, 30% ethanol to afford the diastereomers:

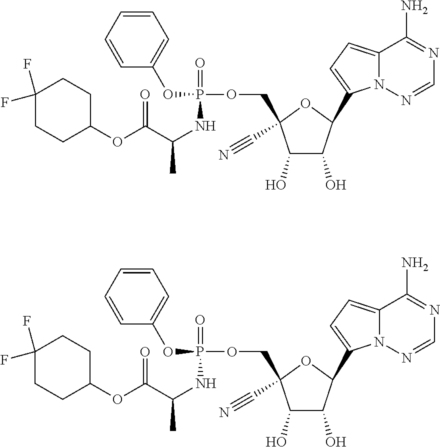 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OC1CCC(F)(F)CC1 ; C[C@H](N[P@](=O)(OC[C@@]1(C#N)O[C@@H](c2ccc3c(N)ncnn23)[C@H](O)[C@@H]1O)Oc1ccccc1)C(=O)OC1CCC(F)(F)CC1]

### Example 175. First Eluting Diastereomer

¹H NMR (400 MHz, Methanol-d4) δ 7.78 (s, 1H), 7.34-7.23 (m, 2H), 7.20-7.11 (m, 3H), 6.85 (d, J=4.5 Hz, 1H), 6.73 (d, J=4.5 Hz, 1H), 5.50 (d, J=5.0 Hz, 1H), 4.63 (t, J=5.3 Hz, 1H), 4.87 (m, 1H, buried by solvent peak), 4.54-4.43 (m, 2H), 4.36 (dd, J=10.9, 5.3 Hz, 1H), 3.97-3.84 (m, 1H), 2.10-1.68 (m, 8H), 1.28 (dd, J=7.1, 1.3 Hz, 3H). ³¹P NMR (162 MHz, Methanol-d4) δ 3.22. ¹⁹F NMR (376 MHz, Methanol-d4) δ −96.61 (d, J=235.4 Hz), −102.50 (d, J=235.6 Hz).

### Example 176. Second Eluting Diastereomer

¹H NMR (400 MHz, Methanol-d4) δ 7.80 (s, 1H), 7.42-7.27 (m, 2H), 7.26-7.13 (m, 3H), 6.84 (d, J=4.5 Hz, 1H), 6.74 (d, J=4.5 Hz, 1H), 5.49 (d, J=4.9 Hz, 1H), 4.81 (s, 1H), 4.61 (t, J=5.3 Hz, 1H), 4.46 (d, J=5.7 Hz, 1H), 4.42 (dd, J=10.9, 6.5 Hz, 1H), 4.34 (dd, J=10.9, 5.5 Hz, 1H), 3.91 (dq, J=9.9, 7.1 Hz, 1H), 2.15-1.69 (m, 8H), 1.27 (dd, J=7.1, 1.1 Hz, 3H). ³¹P NMR (162 MHz, Methanol-d4) δ 3.24. ¹⁹F NMR (376 MHz, Methanol-d4) δ −96.36 (d, J=236.3 Hz), −102.62 (d, J=237.6 Hz).

### Example 177. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl isobutyl carbonate

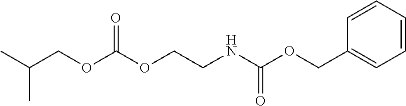 [SMILES: CC(C)COC(=O)OCCNC(=O)OCc1ccccc1]

benzyl (2-((isobutoxycarbonyl)oxy)ethyl)carbamate. N-Cbz-aminoethanol (390 mg, 2 mmol) was dissolved in anhydrous THF (10 mL) and stirred under atmospheric nitrogen. Pyridine (425 uL, 5 mmol) was added in one portion. Isobutyl chloroformate (285 uL, 2.2 mmol) was added dropwise. Reaction was stirred for 15 mins. Reaction was diluted with EtOAc (30 mL) and washed with water (2×20 mL) and followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 7.41-7.28 (m, 5H), 5.11 (m, 3H), 4.22 (t, J=5.2 Hz, 2H), 3.92 (d, J=6.7 Hz, 2H), 3.50 (q, J=5.5 Hz, 2H), 1.97 (dp, J=13.4, 6.7 Hz, 1H), 0.95 (d, J=6.8 Hz, 6H).

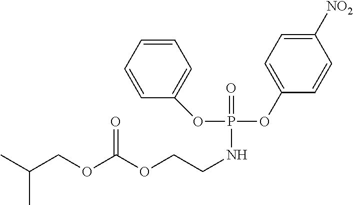 [SMILES: CC(C)COC(=O)OCCNP(=O)(Oc1ccccc1)Oc1ccc([N+](=O)[O-])cc1]

isobutyl (2-(((4-nitrophenoxy)(phenoxy)phosphoryl)amino)ethyl) carbonate. benzyl (2-((isobutoxycarbonyl)oxy)ethyl)carbamate (570 mg, 1.93 mmol) was dissolved in anhydrous THF (12 mL). 10% Pd/C Degussa type was added and the reaction mixture was stirred under atmospheric hydrogen for 3 hrs. Catalyst was filtered and the filtrate was used without purification.

Phenyl dichlorophosphate (344 uL, 2.32 mmol) was dissolved in anhydrous DCM (10 mL) and stirred in an ice bath under atmospheric nitrogen. Above THF solution was added to the reaction dropwise and then stirred for 50 mins. Triethylamine (350 uL, 2.51 mmol) was added dropwise and then stirred for 30 mins. p-Nitrophenol (242 mg, 1.74 mmol) and triethylamine (350 uL, 2.51 mmol) were added. Ice bath was removed and the reaction mixture was stirred for 14 hrs at RT.

Reaction was diluted with EtOAc (30 mL) and washed with water (2×20 mL) and followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the intermediate. ¹H NMR (400 MHz, Chloroform-d) δ 8.27-8.18 (m, 2H), 7.45-7.30 (m, 4H), 7.28-7.15 (m, 3H), 4.22-4.15 (m, 2H), 3.90 (d, J=6.7 Hz, 2H), 3.51 (m, 1H), 3.41 (m, 2H), 1.95 (m, 1H), 0.94 (d, J=6.7 Hz, 6H). ³¹P NMR (162 MHz, Chloroform-d) δ −1.51 (s). MS m/z=438.9 [M+1]; 437.0 [M−1].

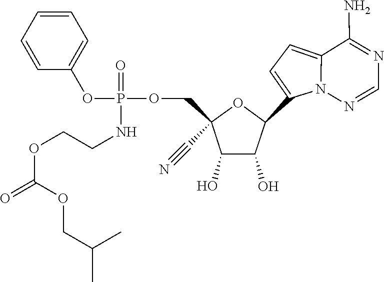 [SMILES: CC(C)COC(=O)OCCNP(=O)(OC[C@@]1(C#N)O[C@@H](c2ccc3c(N)ncnn23)[C@H](O)[C@@H]1O)Oc1ccccc1]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl isobutyl carbonate. Intermediate 4 (50 mg, 0.15 mmol) and isobutyl (2-(((4-nitrophenoxy)(phenoxy)phosphoryl)amino)ethyl) carbonate (73 mg, 0.166 mmol) were mixed and dissolved in anhydrous THF (5 mL). Magnesium chloride (86 mg, 0.906 mmol) was added in one portion and the reaction was stirred at 50° C. for 10 mins. DIPEA (158 uL, 0.906 mmol) was added and the reaction was stirred at 50° C. for 3 hrs. More magnesium chloride (100 mg) was added and stirred at 50° C. for 16 hrs.

Reaction was diluted with EtOAc (20 mL) and washed with water (5×20 mL) and with brine (10 mL). Organic layer was dried over anhydrous sodium sulfate and concentrated under reduced pressure which was then dissolved in MeCN (6 mL). 12 M HCl (aq) (300 uL) was added dropwise. Reaction was stirred for 1 hr. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions having the desired product were combined and concentrated under reduced pressure and the resulting product was then dissolved in MeCN and water and freeze-dried to afford the product. ¹H NMR (400 MHz, Methanol-d4) δ 7.79 (m, 1H), 7.38-7.09 (m, 5H), 6.84 (m, 1H), 6.73 (m, 1H), 5.51 (m, 1H), 4.62 (m, 1H), 4.54-4.26 (m, 3H), 4.04 (m, 2H), 3.85 (m, 2H), 3.18 (m, 2H), 1.88 (m, 1H), 0.96-0.80 (m, 6H). ³¹P NMR (162 MHz, Methanol-d4) δ 5.22 (s), 5.06 (s). MS m/z=591.0 [M+1]; 588.9 [M−1].

### Example 178. cis-4-(trifluoromethyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

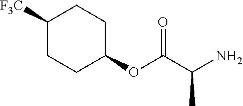 [SMILES: C[C@H](N)C(=O)O[C@H]1CC[C@@H](C(F)(F)F)CC1]

Cis-4-(trifluoromethyl)cyclohexyl L-alaninate. To a mixture of cbz-L-alanine (1.80 g, 8.06 mmol), trans-4-trifluoromethylcyclohexanol (0.9 g, 5.35 mmol), and Ph₃P (3.17 g, 12.10 mmol) in THF (50 mL) was added DIAD (2.38 mL, 12.10 mmol). The mixture was stirred at room temperature for 15 h and concentrated in vacuo. The obtained residue was purified by silica gel chromatography (EtOAc 0 to 30% in hexanes) to give a Cbz-L-alanine cyclohexyl ester, which was dissolved in THF (10 mL) and 20% palladium hydroxide on carbon (250 mg) was added. The resulting mixture was stirred under H₂ for 2 h and filtered. The filtrate was concentrated in vacuo, dried under high vacuum, and used next reaction (747 mg, 39%). MS m/z=240 [M+H].

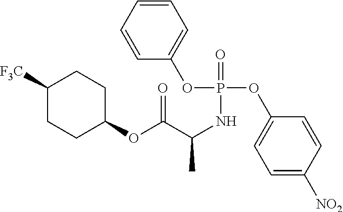 [SMILES: C[C@H](NP(=O)(Oc1ccccc1)Oc1ccc([N+](=O)[O-])cc1)C(=O)O[C@H]1CC[C@@H](C(F)(F)F)CC1]

Cis-4-(trifluoromethyl)cyclohexyl ((4-nitrophenoxy)(phenoxy)phosphoryl)-L-alaninate. The intermediate was prepared as isomeric mixture from cis-4-(trifluoromethyl)cyclohexyl L-alaninate (747 mg, 3.13 mmol) in a manner similar to that described for Intermediate 25. ¹H NMR (400 MHz, Chloroform-d) δ 8.22 (m, 2H), 7.46-7.29 (m, 4H), 7.29-7.13 (m, 3H), 5.05 (m, 1H), 4.27-4.08 (m, 1H), 3.90 (m, 1H), 2.05 (m, 1H), 1.95 (m, 2H), 1.77 (m, 2H), 1.56 (m, 4H), 1.43 (m, 3H). ³¹P NMR (162 MHz, Chloroform-d) δ −3.10. ¹⁹F NMR (376 MHz, Chloroform-d) δ −74.36 (dd, J=8.5, 4.8 Hz). MS m/z=517 [M+H].

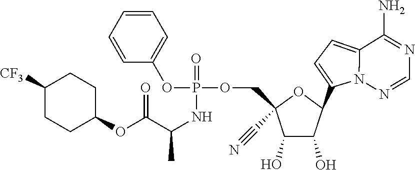 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1]

Cis-4-(trifluoromethyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was obtained from cis-4-(trifluoromethyl)cyclohexyl ((4-nitrophenoxy)(phenoxy)phosphoryl)-L-alaninate (117 mg, 0.23 mmol) and Intermediate 4 (50 mg, 0.15 mmol) in a manner similar to that described for Example 3.

The product was separated by SFC using 30% ethanol (AD-H4.6×100m column) to afford the first eluting diastereomer and the second eluting diastereomer.

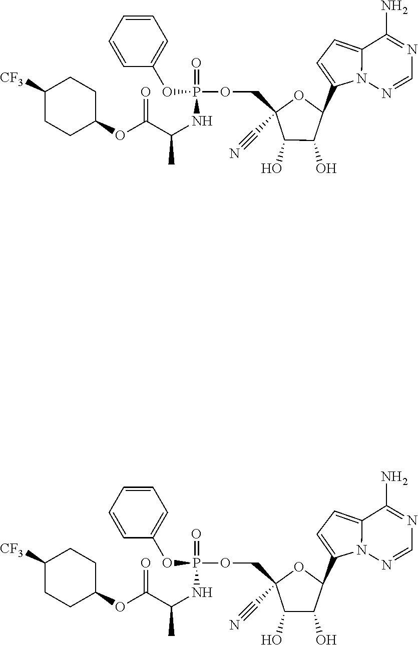 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1 ; C[C@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@@H](C(F)(F)F)CC1]

### Example 179. First Eluting Diastereomer

¹H NMR (400 MHz, Methanol-d4) δ 7.78 (s, 1H), 7.29 (dd, J=8.8, 7.1 Hz, 2H), 7.24-7.12 (m, 3H), 6.84 (d, J=4.6 Hz, 1H), 6.73 (d, J=4.6 Hz, 1H), 5.50 (d, J=5.1 Hz, 1H), 4.97 (s, 1H), 4.63 (t, J=5.3 Hz, 1H), 4.49 (dd, J=12.5, 5.7 Hz, 2H), 4.36 (dd, J=11.0, 5.3 Hz, 1H), 4.02-3.86 (m, 1H), 2.17 (m, 1H), 1.92 (m, 2H), 1.71 (m, 2H), 1.58 (m, 4H), 1.31 (d, J=7.2, 3H). ³¹P NMR (162 MHz, Methanol-d4) δ 3.31. ¹⁹F NMR (377 MHz, Methanol-d4) δ −75.89 (d, J=8.6 Hz). MS m/z=669 [M+1].

### Example 180. Second Eluting Diastereomer

¹H NMR (400 MHz, Methanol-d4) δ 7.80 (s, 1H), 7.39-7.27 (m, 2H), 7.28-7.09 (m, 3H), 6.84 (d, J=4.5 Hz, 1H), 6.73 (d, J=4.5 Hz, 1H), 5.48 (d, J=5.2 Hz, 1H), 4.89 (m, 1H, buried by solvent peak), 4.61 (t, J=5.3 Hz, 1H), 4.46 (d, J=5.6 Hz, 1H), 4.42 (dd, J=10.9, 6.4 Hz, 1H), 4.34 (dd, J=10.9, 5.4 Hz, 1H), 3.94 (dq, J=9.8, 7.1 Hz, 1H), 2.26-2.03 (m, 1H), 1.96-1.79 (m, 2H), 1.68 (d, J=10.0 Hz, 2H), 1.62-1.41 (m, 4H), 1.29 (d, J=7.2 Hz, 3H). ³¹P NMR (162 MHz, Methanol-d4) δ 3.30. ¹⁹F NMR (377 MHz, Methanol-d4) δ −75.87 (d, J=8.6 Hz). MS m/z=669 [M+1].

### Example 181. 2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl dodecanoate

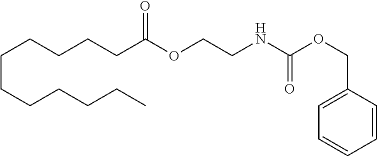 [SMILES: CCCCCCCCCCCC(=O)OCCNC(=O)OCc1ccccc1]

2-(((benzyloxy)carbonyl)amino)ethyl dodecanoate. Lauric acid (481 mg, 2.4 mmol) was mixed with anhydrous acetonitrile (10 mL). EDCI (460 mg, 2.4 mmol) was added in one portion and the reaction was stirred for 15 mins. N-Cbz-aminoethanol (390 mg, 2 mmol) was added. DMAP (293 mg, 2.4 mmol) was then added and the reaction was stirred for 16 hrs.

Reaction was diluted with EtOAc (40 mL) and washed with 5% aqueous citric acid solution (2×10 mL), followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the intermediate. ¹H NMR (400 MHz, Chloroform-d) δ 7.36 (m, 5H), 5.11 (s, 2H), 4.99 (s, 1H), 4.15 (t, J=5.3 Hz, 2H), 3.47 (q, J=5.6 Hz, 2H), 2.32 (m, 2H), 1.72-1.52 (m, 2H), 1.40-1.16 (m, 16H), 0.88 (t, J=6.6 Hz, 3H). MS m/z=377.8 [M+1].

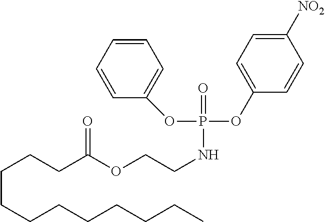 [SMILES: CCCCCCCCCCCC(=O)OCCNP(=O)(Oc1ccccc1)Oc1ccc([N+](=O)[O-])cc1]

2-(((4-Nitrophenoxy)(phenoxy)phosphoryl)amino)ethyl dodecanoate. 2-(((benzyloxy)carbonyl)amino)ethyl dodecanoate (439 mg, 1.16 mmol) was dissolved in anhydrous THF (12 mL). 10% Pd/C Degussa type was added and the reaction mixture was stirred under atmospheric hydrogen for 2 hrs. Catalyst was filtered and the filtrate was used without purification.

Phenyl dichlorophosphate (190 uL, 1.28 mmol) was dissolved in anhydrous DCM (10 mL) and stirred in an ice bath under atmospheric nitrogen. Above THF solution was added to the reaction dropwise and then stirred for 30 mins. Triethylamine (194 uL, 1.39 mmol) was added dropwise and then stirred for 30 mins. p-Nitrophenol (145 mg, 1.04 mmol) and triethylamine (194 uL, 1.39 mmol) were added. Ice bath was removed and the reaction mixture was stirred for 16 hrs at RT.

Reaction was diluted with EtOAc (30 mL) and washed with water (4×20 mL) and followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions having the desired product were combined and concentrated under reduced pressure to afford the intermediate. ¹H NMR (400 MHz, Chloroform-d) δ 8.28-8.19 (m, 2H), 7.45-7.30 (m, 4H), 7.30-7.16 (m, 3H), 4.15 (dt, J=10.1, 4.8 Hz, 2H), 3.52 (d, J=5.4 Hz, 1H), 3.38 (dt, J=10.6, 4.9 Hz, 2H), 2.29 (m, 2H), 1.59 (m, 2H), 1.27 (m, 16H), 0.88 (t, J=6.7 Hz, 3H). ³¹P NMR (162 MHz, Chloroform-d) δ −1.51 (s). MS m/z=520.9 [M+1]; 519.2 [M−1].

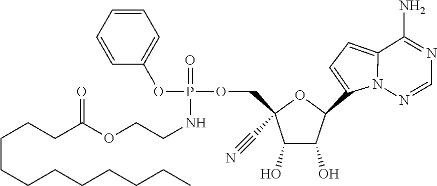 [SMILES: CCCCCCCCCCCC(=O)OCCNP(=O)(OC[C@@]1(C#N)O[C@@H](c2ccc3c(N)ncnn23)[C@H](O)[C@@H]1O)Oc1ccccc1]

2-(((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)amino)ethyl dodecanoate. Intermediate 4 (50 mg, 0.15 mmol) and 2-(((4-nitrophenoxy)(phenoxy)phosphoryl)amino)ethyl dodecanoate (86 mg, 0.166 mmol) were mixed and dissolved in anhydrous THF (5 mL). Magnesium chloride (86 mg, 0.906 mmol) was added in one portion and the reaction was stirred at 50° C. for 10 mins. DIPEA (158 uL, 0.906 mmol) was added and the reaction was stirred at 45° C. for 16 hrs.

Reaction was diluted with EtOAc (20 mL) and washed with water (5×20 mL) and with brine (10 mL). Organic layer was dried over anhydrous sodium sulfate and concentrated under reduced pressure which was then dissolved in MeCN (3 mL). 12 M HCl (aq) (300 uL) was added dropwise. Reaction was stirred for 3 hrs. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-3-8% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.78 (m, 1H), 7.38-7.09 (m, 5H), 6.84 (m, 1H), 6.72 (m, 1H), 5.51 (d, J=4.9 Hz, 1H), 4.62 (m, 1H), 4.48 (t, J=6.0 Hz, 1H), 4.45-4.28 (m, 2H), 3.99 (m, 2H), 3.22-3.07 (m, 2H), 2.24 (m, 2H), 1.52 (m, 2H), 1.26 (m, 16H), 0.89 (t, J=6.8 Hz, 3H). ³¹P NMR (162 MHz, Methanol-d4) δ 5.22 (s), 5.06 (s). MS m/z=673.1 [M+1]; 671.0 [M−1].

### Example 182

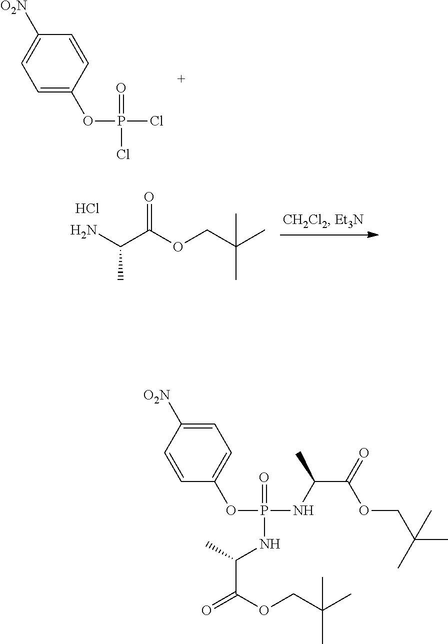 [SMILES: O=[N+]([O-])c1ccc(OP(=O)(Cl)Cl)cc1 ; Cl ; C[C@H](N)C(=O)OCC(C)(C)C ; C[C@H](NP(=O)(N[C@@H](C)C(=O)OCC(C)(C)C)Oc1ccc([N+](=O)[O-])cc1)C(=O)OCC(C)(C)C]

4-Nitrophenyl dichlorophosphate (256 mg, 1 mmol) was dissolved in anhydrous DCM (10 mL) and stirred under atmospheric nitrogen in an ice bath. Neopentyl L-alaninate hydrochloride (391 mg, 2 mmol) was added in one portion. Triethylamine (698 uL, 5 mmol) was added dropwise and the reaction was stirred for 16 hrs.

Reaction was diluted with DCM (20 mL) and washed with 2% aqueous citric acid solution (10 mL). Organic layer was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions having the desired product were combined and concentrated under reduced pressure to afford the intermediate. ¹H NMR (400 MHz, Chloroform-d) δ 8.21 (d, J=9.0 Hz, 2H), 7.38 (d, J=9.0 Hz, 2H), 4.21-4.00 (m, 2H), 3.88 (dd, J=10.5, 1.7 Hz, 2H), 3.77 (dd, J=10.5, 3.3 Hz, 2H), 3.59 (t, J=10.0 Hz, 2H), 1.44 (m, 6H), 0.93 (m, 18H). ³¹P NMR (162 MHz, Chloroform-d) δ 7.98 (s). MS m/z=500.0 [M−1].

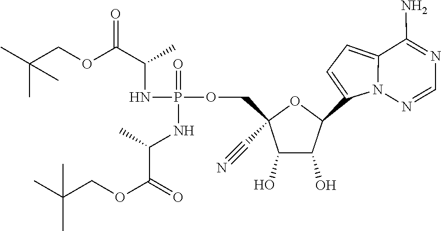 [SMILES: C[C@H](NP(=O)(N[C@@H](C)C(=O)OCC(C)(C)C)OC[C@@]1(C#N)O[C@@H](c2ccc3c(N)ncnn23)[C@H](O)[C@@H]1O)C(=O)OCC(C)(C)C]

Intermediate 4 (50 mg, 0.15 mmol) and the above intermediate (83 mg, 0.166 mmol) were mixed and dissolved in anhydrous THF (5 mL). Magnesium chloride (72 mg, 0.755 mmol) was added in one portion and the reaction was stirred at RT for 10 mins. DIPEA (66 uL, 0.378 mmol) was added and the reaction was stirred at RT for 20 hrs.

Reaction was diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 1 hr. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions with the desired product were combined and concentrated under reduced pressure to give oil which was then dissolved in MeCN and water and freeze-dried to afford the product. ¹H NMR (400 MHz, Methanol-d4) δ 7.82 (s, 1H), 6.85 (d, J=4.5 Hz, 1H), 6.77 (d, J=4.5 Hz, 1H), 5.51 (d, J=4.8 Hz, 1H), 4.61 (t, J=5.3 Hz, 1H), 4.50 (d, J=5.7 Hz, 1H), 4.32 (dd, J=11.1, 7.1 Hz, 1H), 4.22 (dd, J=11.1, 5.8 Hz, 1H), 4.03-3.88 (m, 2H), 3.88-3.63 (m, 4H), 1.35 (d, J=7.2 Hz, 3H), 1.29 (d, J=7.2 Hz, 3H), 0.93 (s, 9H), 0.90 (s, 9H). ³¹P NMR (162 MHz, Methanol-d4) δ 13.57 (s). MS m/z=654.0 [M+1]; 652.1 [M−1].

### Example 183. 4,4-dimethylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

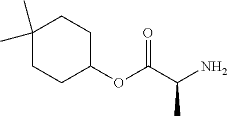

4,4-Dimethylcyclohexyl L-alaninate. The intermediate was prepared from Cbz-1-alanine (1000 mg, 4.48 mmol) and 4,4-dimethylcyclohexanol (690 mg, 5.38 mmol) in a manner similar to that described for Intermediate 26. ¹H NMR (400 MHz, Chloroform-d) δ 4.76 (dt, J=8.9, 4.6 Hz, 1H), 3.53 (q, J=7.0 Hz, 1H), 1.75 (m, 2H), 1.58 (m, 2H), 1.42 (m, 2H), 1.38-1.17 (m, 5H), 0.94 (s, 3H), 0.92 (s, 3H).

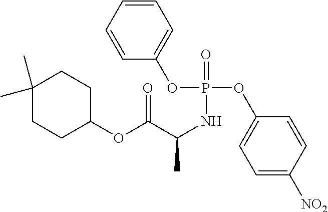 [SMILES: C[C@H](NP(=O)(Oc1ccccc1)Oc1ccc([N+](=O)[O-])cc1)C(=O)OC1CCC(C)(C)CC1]

4,4-dimethylcyclohexyl ((4-nitrophenoxy)(phenoxy)phosphoryl)-L-alaninate. The intermediate was prepared as isomeric mixture from 4,4-dimethylcyclohexyl L-alaninate (186 mg, 0.93 mmol) by the same method used for Intermediate 35. ¹H NMR (400 MHz, Chloroform-d) δ 8.22 (m, 2H), 7.45-7.30 (m, 4H), 7.28-7.15 (m, 3H), 4.74 (m, 1H), 4.21-4.01 (m, 1H), 3.90 (m, 1H), 1.80-1.62 (m, 2H), 1.62-1.49 (m, 2H), 1.46-1.34 (m, 5H), 1.24 (m, 2H), 0.92 (s, 3H), 0.91 (s, 3H). ³¹P NMR (162 MHz, Chloroform-d) δ −2.96, −3.02. MS m/z=477 [M+1].

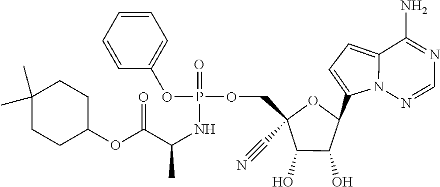 [SMILES: C[C@H](NP(=O)(OC[C@@]1(C#N)O[C@@H](c2ccc3c(N)ncnn23)[C@H](O)[C@@H]1O)Oc1ccccc1)C(=O)OC1CCC(C)(C)CC1]

4,4-dimethylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was obtained from 4,4-dimethylcyclohexyl ((4-nitrophenoxy)(phenoxy)phosphoryl)-L-alaninate (108 mg, 0.23 mmol) and Intermediate 4 (50 mg, 0.15 mmol) in a manner similar to that described for Example 3. ¹H NMR (400 MHz, Methanol-d4) δ 7.78 (m, 1H), 7.38-7.09 (m, 5H), 6.84 (m, 1H), 6.72 (m, 1H), 5.51 (m, 1H), 4.62 (m, 2H), 4.53-4.29 (m, 3H), 3.96-3.78 (m, 1H), 1.77-1.57 (m, 2H), 1.58-1.43 (m, 2H), 1.44-1.29 (m, 2H), 1.29-1.08 (m, 5H), 0.88 (m, 6H). ³¹P NMR (162 MHz, Methanol-d₄) δ 3.29, 3.27. MS m/z=629 [M+H].

### Example 184. 1-acetylpiperidin-4-yl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)alaninate

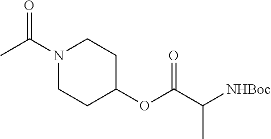 [SMILES: CC(=O)N1CCC(OC(=O)C(C)NC(=O)OC(C)(C)C)CC1]

1-acetylpiperidin-4-yl (tert-butoxycarbonyl)alaninate. This intermediate was prepared in a manner similar to that described for Example 117. ¹H NMR (400 MHz, DMSO-d) δ 7.26 (d, J=7.3 Hz, 1H), 4.90 (tt, J=7.6, 3.8 Hz, 1H), 4.03-3.97 (m, 1H), 3.67-3.50 (m, 3H), 3.41-3.31 (m, 3H), 2.02-1.96 (m, 4H), 1.77 (d, J=39.0 Hz, 2H), 1.61-1.40 (m, 1H), 1.37 (s, 9H), 1.24 (d, J=7.3 Hz, 4H).

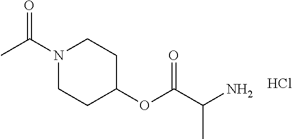

1-acetylpiperidin-4-yl alaninate hydrochloride. The intermediate was prepared in a manner similar to that described for Intermediate 13. ¹H NMR (400 MHz, DMSO-d6) δ 8.57 (s, 3H), 5.00 (dt, J=7.2, 3.6 Hz, 1H), 4.11-3.99 (m, 1H), 3.55 (m, 2H), 3.38 (dtd, J=13.7, 8.7, 7.3, 3.6 Hz, 2H), 1.99 (s, 3H), 1.90-1.70 (m, 2H), 1.65-1.45 (m, 2H), 1.41 (d, J=7.2 Hz, 3H).

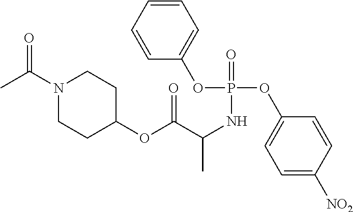 [SMILES: CC(=O)N1CCC(OC(=O)C(C)NP(=O)(Oc2ccccc2)Oc2ccc([N+](=O)[O-])cc2)CC1]

1-acetylpiperidin-4-yl-((4-nitrophenoxy)(phenoxy)phosphoryl)alaninate. The intermediate was prepared in a manner similar to that described for Intermediate 35. ¹H NMR (400 MHz, DMSO-d6) δ 8.37-8.21 (m, 2H), 7.54-7.32 (m, 4H), 7.32-7.11 (m, 3H), 6.69 (ddd, J=13.6, 10.0, 5.6 Hz, 1H), 4.83 (dd, J=8.0, 4.0 Hz, 1H), 4.07-3.87 (m, 1H), 3.69-3.37 (m, 2H), 3.29 (m, 2H), 1.95 (s, 3H), 1.70 (d, J=36.7 Hz, 2H), 1.40 (d, J=31.8 Hz, 2H), 1.27-1.19 (m, 3H). ³¹P NMR (162 MHz, DMSO-d6) δ −1.28, −1.42. MS m/z=492.08 [M+1].

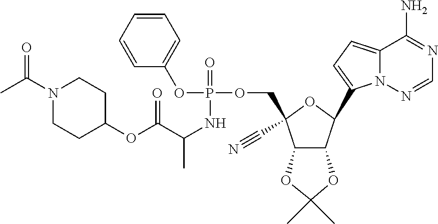 [SMILES: CC(=O)N1CCC(OC(=O)C(C)NP(=O)(OC[C@@]2(C#N)O[C@@H](c3ccc4c(N)ncnn34)[C@@H]3OC(C)(C)O[C@@H]32)Oc2ccccc2)CC1]

1-acetylpiperidin-4-yl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)alaninate. To a mixture of Intermediate 4 (50.0 mg, 0.151 mmol), 1-acetylpiperidin-4-yl-((4-nitrophenoxy)(phenoxy)phosphoryl)alaninate (164 mg, 0.332 mmol), and magnesium chloride (144 mg, 1.51 mmol) was added acetonitrile (8 mL) at room temperature. The resulting suspension was warmed to 50° C., and was allowed to stir for 10 min. N, N-Diisopropylethylamine (0.26 mL, 1.51 mmol) was then added and the resulting mixture was stirred at 50° C. for 4 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified via SiO₂ column chromatography (40 g SiO₂ Combiflash HP Gold Column, 100% Dichloromethane-14% Methanol in dichloromethane) to afford the product. ¹H NMR (400 MHz, DMSO-d6) δ 7.84 (d, J=16.8 Hz, 3H), 7.32 (dt, J=12.1, 7.9 Hz, 2H), 7.24-7.04 (m, 3H), 6.89-6.72 (m, 2H), 6.24-6.06 (m, 1H), 5.61 (dd, J=5.4, 3.6 Hz, 1H), 5.27 (dd, J=6.6, 3.8 Hz, 1H), 5.06 (t, J=6.9 Hz, 1H), 4.82 (s, 1H), 4.37-4.17 (m, 1H), 3.82 (q, J=9.4 Hz, 1H), 3.67-3.38 (m, 2H), 3.29 (s, 3H), 1.95 (d, J=2.6 Hz, 3H), 1.63 (d, J=3.1 Hz, 5H), 1.33 (d, J=3.5 Hz, 4H), 1.22 (dt, J=18.3, 6.8 Hz, 5H). MS m/z=684.18 [M+1].

### Example 185. 1-acetylpiperidin-4-yl ((((2R,3S,4R,5S)-5-(4-aminopyrrolo[2,1-f][1,2,4]triazin-7-yl)-2-cyano3,4-dihydroxytetrahydrofuran-2-yl)methoxy)(phenoxy)phosphoryl)alaninate

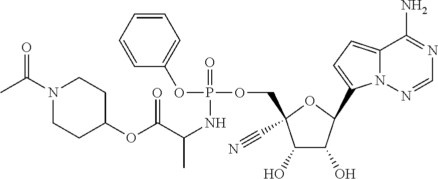 [SMILES: CC(=O)N1CCC(OC(=O)C(C)NP(=O)(OC[C@@]2(C#N)O[C@@H](c3ccc4c(N)ncnn34)[C@H](O)[C@@H]2O)Oc2ccccc2)CC1]

To a mixture of Example 184 (0.09 g, 0.132 mmol) in acetonitrile (2 mL) at 0° C. was added concentrated hydrochloric acid (0.1 mL, 2.743 mmol) and the reaction mixture was stirred at room temperature for 1 h. After 1 h. the reaction mixture was cooled in an ice bath and was diluted with saturated sodium bicarbonate solution (1 mL). The resulting mixture was purified by preparative HPLC (Phenominex Synergi 4 u Hydro-RR 80 Å 150×30 mm column, 5%-60% acetonitrile/water gradient in 30 min run) to afford the product. ¹H NMR (400 MHz, DMSO-d6) δ 7.88-7.66 (m, 3H), 7.45-7.02 (m, 5H), 6.84 (d, J=4.5 Hz, 1H), 6.77-6.66 (m, 1H), 6.22-5.99 (m, 2H), 5.56-5.44 (m, 1H), 5.38 (t, J=5.4 Hz, 1H), 4.94-4.72 (m, 1H), 4.53-4.39 (m, 1H), 4.40-4.08 (m, 3H), 3.94-3.73 (m, 1H), 3.70-3.39 (m, 2H), 1.95 (q, J=2.9, 2.4 Hz, 3H), 1.70 (d, J=32.9 Hz, 2H), 1.55-1.31 (m, 2H), 1.27-1.06 (m, 3H). MS m/z=644.15 [M+1].

### Example 186. trans-4-phenylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

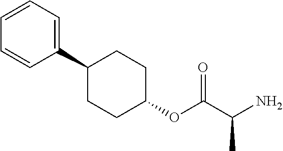 [SMILES: C[C@H](N)C(=O)O[C@H]1CC[C@H](c2ccccc2)CC1]

(1R,4S)-4-phenylcyclohexyl L-alaninate. The intermediate was prepared from Cbz-1-alanine (1000 mg, 4.48 mmol) and trans-4-phenylcyclohexanol (950 mg, 5.38 mmol) in a manner similar to that described for Intermediate 26. MS m/z=248 [M+H].

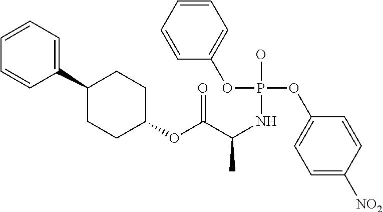

Trans-4-phenylcyclohexyl ((4-nitrophenoxy)(phenoxy) phosphoryl)-L-alaninate. The intermediate was prepared as isomeric mixture from (1R,4S)-4-phenylcyclohexyl L-alaninate (488 mg, 1.97 mmol) in a manner similar to that described for Intermediate 25. ¹H NMR (400 MHz, Chloroform-d) δ 8.23 (m, 2H), 7.45-7.16 (m, 12H), 4.87-4.71 (m, 1H), 4.13 (m, 1H), 3.95-3.82 (m, 1H), 2.51 (m, 1H), 2.05 (m, 2H), 1.96 (m, 2H), 1.68-1.46 (m, 4H), 1.42 (m, 3H). ³¹P NMR (162 MHz, Chloroform-d) δ −3.03, −3.06. MS m/z 525=[M+H].

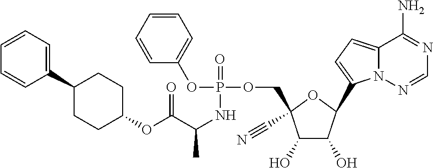 [SMILES: C[C@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](c2ccccc2)CC1]

Trans-4-phenylcyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. The product was obtained from trans-4-phenylcyclohexyl ((4-nitrophenoxy)(phenoxy) phosphoryl)-L-alaninate (131 mg, 0.25 mmol) and Intermediate 4 (55 mg, 0.17 mmol) in a manner similar to that described for Example 3. ¹H NMR (400 MHz, Methanol-d4) δ 7.79 (m, 1H), 7.39-7.06 (m, 10H), 6.85 (m, 1H), 6.74 (m, 1H), 5.52 (d, J=4.9 Hz, 1H), 4.76-4.51 (m, 2H), 4.51-4.33 (m, 3H), 3.97-3.80 (m, 1H), 2.57-2.38 (m, 1H), 2.05-1.92 (m, 2H), 1.91-1.74 (m, 2H), 1.67-1.37 (m, 4H), 1.27 (m, 3H). ³¹P NMR (162 MHz, Methanol-d4) δ 3.32, 3.25. MS m/z=677 [M+1].

### Example 187. ((2R,3S,4R,5S)-5-(4-aminopyrrolo[2,1-f][1,2,4]triazin-7-yl)-2-cyano-3,4-dihydroxytetrahydrofuran-2-yl)methyl phenyl (3-(hexadecyloxy)propyl)phosphoramidate

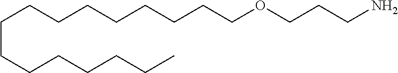 [SMILES: CCCCCCCCCCCCCCCCOCCCN]

3-(Hexadecyloxy)propan-1-amine. 3-(hexadecyloxy)propan-1-ol (300 mg, 1 mmol) was dissolved in anhydrous DMF (5 mL) and stirred under atmospheric nitrogen. Diphenyl phosphoryl azide (259 uL, 1.2 mmol) was added in one portion. DBU (179 uL, 1.2 mmol) was added and the reaction was stirred for 16 hrs.

Reaction was warmed to 90° C. and stirred for 2 hrs. Reaction was cooled to RT, diluted with EtOAc (25 mL) and washed with brine (2×10 mL). Organic layer was dried over anhydrous sodium sulfate and concentrated under reduced pressure. The crude residue was purified via SiO₂ column chromatography (4 g SiO₂ Combiflash HP Gold Column, 0-10% ethyl acetate/hexanes). Fractions with the desired product were combined and concentrated under reduced pressure to an oil which was then dissolve in THF (5 mL). Triphenylphosphine (220 mg, 0.833 mmol) was added and stirred for 20 mins. Water (1 mL) and THF (1 mL) were added to the reaction which was stirred for 24 hrs. Reaction was diluted with EtOAc (20 mL) and washed with saturated sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-5-10% methanol/DCM with 0.1% TEA). Fractions having the desired product were combined and concentrated under reduced pressure to afford the intermediate. ¹H NMR (400 MHz, Chloroform-d) δ 3.48 (t, J=6.2 Hz, 2H), 3.39 (t, J=6.7 Hz, 2H), 2.79 (t, J=6.7 Hz, 2H), 1.71 (p, J=6.5 Hz, 2H), 1.55 (p, J=6.8 Hz, 2H), 1.25 (m, 26H), 0.88 (t, J=6.7 Hz, 3H).

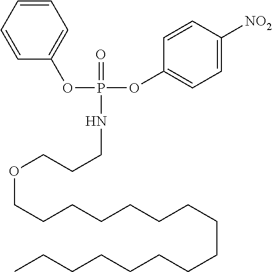 [SMILES: CCCCCCCCCCCCCCCCOCCCNP(=O)(Oc1ccccc1)Oc1ccc([N+](=O)[O-])cc1]

4-Nitrophenyl phenyl (3-(hexadecyloxy)propyl)phosphoramidate. Phenyl dichlorophosphate (104 uL, 0.698 mmol) was dissolved in anhydrous DCM (10 mL) and stirred in an ice bath under atmospheric nitrogen. 3-(hexadecyloxy)propan-1-amine (190 mg, 0.634 mmol) was dissolved in anhydrous DCM (4 mL) and added to the reaction dropwise. Reaction was stirred for 10 mins. Triethylamine (106 uL, 0.761 mmol) was added dropwise and then stirred for 1 hr. p-Nitrophenol (113 mg, 0.571 mmol) and triethylamine (106 uL, 0.761 mmol) were added. Ice bath was removed and the reaction mixture was stirred for 2 hrs at RT.

Reaction was diluted with DCM (20 mL) and washed with 5% aqueous citric acid solution (10 mL) and followed with water (10 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 8.27-8.17 (m, 2H), 7.41 (d, J=8.9 Hz, 2H), 7.35 (t, J=7.8 Hz, 2H), 7.26-7.16 (m, 3H), 3.73 (m, 1H), 3.48 (t, J=5.6 Hz, 2H), 3.35 (t, J=6.7 Hz, 2H), 3.24 (m, 2H), 1.75 (m, 2H), 1.59-1.46 (m, 2H), 1.25 (m, 26H), 0.88 (t, J=6.7 Hz, 3H). ³¹P NMR (162 MHz, Chloroform-d) δ −1.12 (s). MS m/z=577.1 [M+1]; 575.5 [M−1].

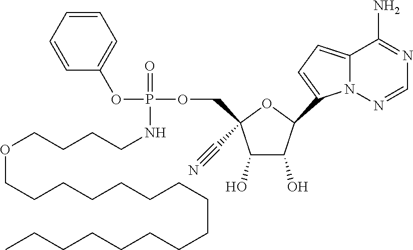 [SMILES: CCCCCCCCCCCCCCCCOCCCCNP(=O)(OC[C@@]1(C#N)O[C@@H](c2ccc3c(N)ncnn23)[C@H](O)[C@@H]1O)Oc1ccccc1]

((2R,3S,4R,5S)-5-(4-aminopyrrolo[2,1-f][1,2,4]triazin-7-yl)-2-cyano-3,4-dihydroxytetrahydrofuran-2-yl)methyl phenyl (3-(hexadecyloxy)propyl)phosphoramidate. Intermediate 4 (75 mg, 0.226 mmol) and 4-nitrophenyl phenyl (3-(hexadecyloxy)propyl)phosphoramidate (131 mg, 0.226 mmol) were mixed and dissolved in anhydrous THF (5 mL). Magnesium chloride (108 mg, 1.13 mmol) was added in one portion and the reaction was stirred for 10 mins. DIPEA (98 uL, 0.565 mmol) was added and the reaction was stirred at 50° C. for 16 hrs. More magnesium chloride (108 mg, 1.13 mmol) was added in one portion and the reaction was stirred at 50° C. for 7 hrs.

Reaction was cooled to RT, diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 1 hr. Reaction was diluted with EtOAc (25 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.78 (m, 1H), 7.31 (m, 2H), 7.18 (m, 3H), 6.85 (m, 1H), 6.73 (m, 1H), 5.51 (m, 1H), 4.62 (m, 1H), 4.49 (m, 1H), 4.45-4.26 (m, 2H), 3.56-3.42 (m, 1H), 3.40-3.23 (m, 4H), 3.07-2.90 (m, 2H), 1.64 (m, 2H), 1.48 (m, 2H), 1.40-1.18 (m, 26H), 0.89 (t, J=6.7 Hz, 3H). ³¹P NMR (162 MHz, Methanol-d4) δ 5.48 (s), 5.31 (s). MS m/z=729.1 [M+1]; 726.8 [M−1].

### Example 188. octyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

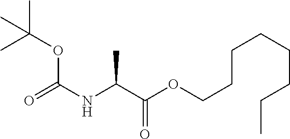 [SMILES: CCCCCCCCOC(=O)[C@H](C)NC(=O)OC(C)(C)C]

Octyl (tert-butoxycarbonyl)-L-alaninate. Boc-L-Ala (567 mg, 3 mmol) was dissolved in anhydrous acetonitrile (15 mL). 1-Octanol (569 uL, 3.6 mmol) was added. EDCI (690 mg, 3.6 mmol) was added in one portion and the reaction was stirred for 15 mins. DMAP (403 mg, 3.3 mmol) was then added and the reaction was stirred for 2 hrs. More 1-Octanol (120 uL) and EDCI (140 mg) were added and the reaction was stirred for 2 hrs. Reaction was diluted with EtOAc (40 mL) and washed with 5% aqueous citric acid solution (2×10 mL), followed with brine (10 mL). Organic layer was dried over anhydrous sodium sulfate and concentrated under reduced pressure. The crude residue was purified via SiO₂ column chromatography (24 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 5.04 (bs, 1H), 4.30 (m, 1H), 4.13 (m, 2H), 1.64 (m, 2H), 1.44 (s, 9H), 1.39 (d, J=7.2 Hz, 3H), 1.27 (m, 10H), 0.88 (t, J=6.8 Hz, 3H).

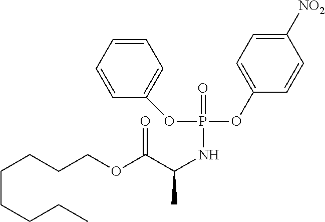 [SMILES: CCCCCCCCOC(=O)[C@H](C)NP(=O)(Oc1ccccc1)Oc1ccc([N+](=O)[O-])cc1]

Octyl ((4-nitrophenoxy)(phenoxy)phosphoryl)-L-alaninate. Octyl (tert-butoxycarbonyl)-L-alaninate (674 mg, 2.24 mmol) was dissolved in anhydrous 4 M HCl in dioxane (15 mL). The reaction was stirred for 2 hrs. The reaction was concentrated under reduced pressure. The resulting solid was dissolved in DCM (20 mL) and concentrated under reduced pressure.

Phenyl dichlorophosphate (366 uL, 2.46 mmol) was dissolved in anhydrous DCM (15 mL) and stirred in an ice bath under atmospheric nitrogen. Above de-Boc material was dissolved in anhydrous DCM (10 mL) and added to the reaction dropwise. The reaction was stirred for 30 mins. Triethylamine (750 uL, 5.4 mmol) was added dropwise and then stirred for 60 mins. p-Nitrophenol (280 mg, 2.02 mmol) and triethylamine (375 uL, 2.69 mmol) were added. Ice bath was removed and the reaction mixture was stirred for 16 hrs at RT.

Reaction was diluted with EtOAc (40 mL) and washed with water (3×20 mL) and followed with brine (10 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the product. ¹H NMR (400 MHz, Chloroform-d) δ 8.22 (m, 2H), 7.48-7.29 (m, 4H), 7.29-7.12 (m, 3H), 4.22-4.02 (m, 3H), 3.94-3.79 (m, 1H), 1.60 (m, 2H), 1.41 (m, 3H), 1.35-1.16 (m, 10H), 0.88 (t, J=6.6 Hz, 3H). ³¹P NMR (162 MHz, Chloroform-d) δ −3.07 (s), −3.11 (s). MS m/z=479.0 [M+1]; 477.2 [M−1].

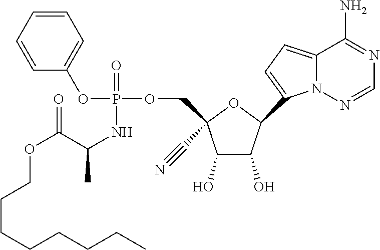 [SMILES: CCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

Octyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.151 mmol) and octyl ((4-nitrophenoxy)(phenoxy)phosphoryl)-L-alaninate (79 mg, 0.166 mmol) were mixed and dissolved in anhydrous THF (5 mL). Magnesium chloride (72 mg, 0.755 mmol) was added in one portion and the reaction was stirred at 50° C. for 10 mins. DIPEA (66 uL, 0.378 mmol) was added and the reaction was stirred at 50° C. for 3 hrs. More magnesium chloride (80 mg) and DIPEA (66 uL, 0.378 mmol) were added and the reaction was stirred at 50° C. for 16 hrs.

Reaction was cooled to RT, diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 1 hr. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-5% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.78 (m, 1H), 7.38-7.07 (m, 5H), 6.84 (m, 1H), 6.72 (m, 1H), 5.51 (m, 1H), 4.62 (m, 1H), 4.55-4.29 (m, 3H), 4.13-3.82 (m, 3H), 1.54 (m, 2H), 1.35-1.15 (m, 13H), 0.87 (t, J=6.8 Hz, 3H). ³¹P NMR (162 MHz, Methanol-d4) δ 3.25 (s). MS m/z=631.1 [M+1]; 629.1 [M−1].

### Example 189. (R)-2-(benzyloxy)-3-(octadecyloxy)propyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)alaninate

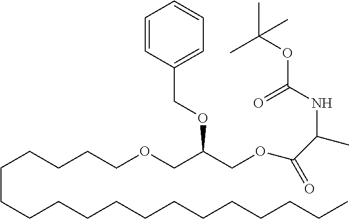 [SMILES: CCCCCCCCCCCCCCCCCCOC[C@H](COC(=O)C(C)NC(=O)OC(C)(C)C)OCc1ccccc1]

(R)-2-(benzyloxy)-3-(octadecyloxy)propyl (tert-butoxycarbonyl)alaninate. The intermediate was prepared in a manner similar to that described for Example 117. ¹H NMR (400 MHz, DMSO-d6) δ 7.41-7.17 (m, 5H), 4.65-4.49 (m, 2H), 4.23 (dd, J=11.6, 4.0 Hz, 1H), 4.02 (ddd, J=11.2, 8.8, 4.9 Hz, 2H), 3.77-3.61 (m, 1H), 3.45 (d, J=5.3 Hz, 2H), 3.36 (t, J=6.5 Hz, 2H), 1.45 (t, J=6.7 Hz, 2H), 1.35 (s, 7H), 1.21 (s, 27H), 0.92-0.70 (m, 2H).

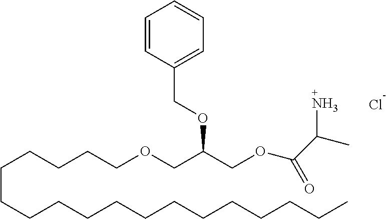 [SMILES: CCCCCCCCCCCCCCCCCCOC[C@H](COC(=O)C(C)N)OCc1ccccc1 ; Cl]

1-((R)-2-(benzyloxy)-3-(octadecyloxy)propoxy)-1-oxopropan-2-aminium chloride. The intermediate was prepared in a manner similar to that described for Intermediate 35. ¹H NMR (400 MHz, DMSO-d6) δ 8.56 (s, 3H), 7.44-7.15 (m, 5H), 4.59 (s, 2H), 4.37 (dd, J=11.6, 3.8 Hz, 1H), 4.21-3.97 (m, 2H), 3.76 (dd, J=5.6, 3.8 Hz, 1H), 3.48 (d, J=5.3 Hz, 2H), 3.37 (t, J=6.5 Hz, 2H), 1.42 (dd, J=25.5, 7.0 Hz, 5H), 1.21 (s, 28H), 0.83 (t, J=6.7 Hz, 3H).

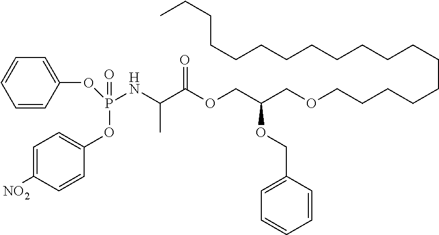 [SMILES: CCCCCCCCCCCCCCCCCCOC[C@H](COC(=O)C(C)NP(=O)(Oc1ccccc1)Oc1ccc([N+](=O)[O-])cc1)OCc1ccccc1]

(R)-2-(benzyloxy)-3-(octadecyloxy)propyl ((4-nitrophenoxy)(phenoxy)phosphoryl)alaninate. The intermediate was prepared in a manner similar to that described for Intermediate 35. ¹H NMR (400 MHz, DMSO-d6) δ 8.35-8.14 (m, 2H), 7.50-7.31 (m, 4H), 7.31-7.12 (m, 8H), 6.71 (dt, J=13.6, 10.2 Hz, 1H), 4.55 (d, J=2.3 Hz, 2H), 4.18 (dt, J=11.6, 4.3 Hz, 1H), 4.10-3.95 (m, 2H), 3.66 (t, J=5.0 Hz, 1H), 3.42 (d, J=5.3 Hz, 2H), 1.42 (t, J=6.7 Hz, 2H), 1.20 (d, J=3.6 Hz, 36H), 0.92-0.75 (m, 3H). ³¹P NMR (162 MHz, DMSO-d6) δ −1.28, −1.56.

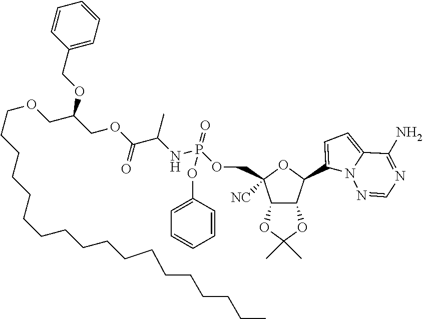 [SMILES: CCCCCCCCCCCCCCCCCCOC[C@H](COC(=O)C(C)NP(=O)(OC[C@@]1(C#N)O[C@@H](c2ccc3c(N)ncnn23)[C@@H]2OC(C)(C)O[C@@H]21)Oc1ccccc1)OCc1ccccc1]

(R)-2-(benzyloxy)-3-(octadecyloxy)propyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)alaninate. To a mixture of Intermediate 4 (50.0 mg, 0.151 mmol), (R)-2-(benzyloxy)-3-(octadecyloxy)propyl ((4-nitrophenoxy)(phenoxy)phosphoryl)alaninate (177 mg, 0.226 mmol), and magnesium chloride (100 mg, 1.056 mmol) was added acetonitrile (8 mL) at room temperature. The resulting suspension was warmed to 50° C., and was allowed to stir for 10 min. N,N-Diisopropylethylamine (0.184 mL, 1.056 mmol) was then added and the resulting mixture was stirred at 50° C. for 3 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified via SiO₂ column chromatography (40 g SiO₂ Combiflash HP Gold Column, 0-100% ethyl acetate/hexanes) to afford the product. ¹H NMR (400 MHz, Acetonitrile-d3) δ 7.89 (s, 1H), 7.47-7.06 (m, 8H), 6.85-6.67 (m, 2H), 6.26 (s, 2H), 5.66 (d, J=3.3 Hz, 1H), 5.27 (dt, J=6.4, 3.1 Hz, 1H), 5.08 (d, J=6.6 Hz, 1H), 4.60 (dd, J=6.7, 2.0 Hz, 2H), 4.49-4.16 (m, 3H), 4.15-3.87 (m, 1H), 3.72 (dd, J=10.3, 5.1 Hz, 1H), 3.49 (t, J=5.8 Hz, 2H), 3.45-3.32 (m, 2H), 1.71 (d, J=2.4 Hz, 3H), 1.52 (t, J=6.6 Hz, 2H), 1.38 (d, J=3.7 Hz, 3H), 1.28 (d, J=2.1 Hz, 33H), 1.02-0.79 (m, 3H). MS m/z=976.31 [M+1].

### Example 190. (R)-2-(benzyloxy)-3-(octadecyloxy)propyl ((((2R,3S,4R,5S)-5-(4-aminopyrrolo[2,1-f][1,2,4]triazin-7-yl)-2-cyano3,4-dihydroxytetrahydrofuran-2-yl)methoxy)(phenoxy)phosphoryl)alaninate

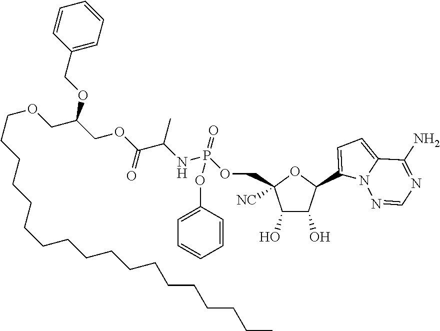 [SMILES: CCCCCCCCCCCCCCCCCCOC[C@H](COC(=O)C(C)NP(=O)(OC[C@@]1(C#N)O[C@@H](c2ccc3c(N)ncnn23)[C@H](O)[C@@H]1O)Oc1ccccc1)OCc1ccccc1]

To a mixture of Example 189 (0.1 g, 0.226 mmol) in acetonitrile (2 mL) at 0° C. was added concentrated hydrochloric acid (0.1 mL, 2.743 mmol) and the reaction mixture was stirred at room temperature for 1 h. After 1 h the reaction mixture was cooled in an ice bath and was diluted with water. Neutralized the solution with 2 N sodium hydroxide and extracted with ethyl acetate. Organic layer was separated, dried over sodium sulfate, filtered and concentrated. The residue obtained was purified by SiO₂ column chromatography (12 g SiO₂ Combiflash HP Gold Column, 100% Dichloromethane-8% Methanol in dichloromethane) to afford the product. ¹H NMR (400 MHz, DMSO-d6) δ 7.85 (m, 3H), 7.44-7.04 (m, 10H), 6.86 (d, J=4.5 Hz, 1H), 6.72 (dd, J=4.5, 2.9 Hz, 1H), 6.27-6.01 (m, 2H), 5.60-5.20 (m, 2H), 4.54 (dd, J=7.6, 2.6 Hz, 2H), 4.45 (t, J=5.8 Hz, 1H), 4.36-4.07 (m, 4H), 4.07-3.77 (m, 1H), 3.74-3.61 (m, 1H), 3.52-3.19 (m, 4H), 1.53-1.36 (m, 2H), 1.20 (d, J=3.3 Hz, 33H), 0.94-0.75 (m, 3H). MS m/z=936.08 [M+1].

### Example 191. dodecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

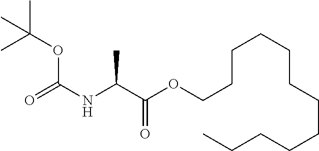 [SMILES: CCCCCCCCCCCCOC(=O)[C@H](C)NC(=O)OC(C)(C)C]

Dodecyl (tert-butoxycarbonyl)-L-alaninate. Boc-L-Ala (567 mg, 3 mmol) was dissolved in anhydrous acetonitrile (15 mL). 1-Dodecanol (818 uL, 3.6 mmol) was added. EDCI (690 mg, 3.6 mmol) was added in one portion and the reaction was stirred for 15 mins. DMAP (403 mg, 3.3 mmol) was then added and the reaction was stirred for 2 hrs. Reaction was diluted with EtOAc (40 mL) and washed with 5% aqueous citric acid solution (2×10 mL), followed with brine (10 mL). Organic layer was dried over anhydrous sodium sulfate and concentrated under reduced pressure. The crude residue was purified via SiO₂ column chromatography (24 g SiO₂ Combiflash HP Gold Column, 0-10% ethyl acetate/hexanes). Fractions having the desired product were combined and concentrated under reduced pressure to afford the intermediate. ¹H NMR (400 MHz, Chloroform-d) δ 5.04 (m, 1H), 4.29 (m, 1H), 4.19-4.04 (m, 2H), 1.62 (m, 2H), 1.44 (s, 9H), 1.38 (d, J=7.2 Hz, 3H), 1.26 (m, 18H), 0.88 (t, J=6.7 Hz, 3H).

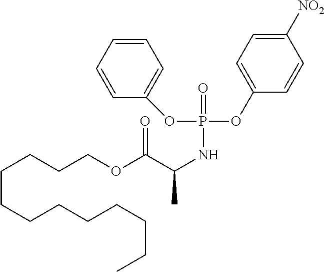 [SMILES: CCCCCCCCCCCCOC(=O)[C@H](C)NP(=O)(Oc1ccccc1)Oc1ccc([N+](=O)[O-])cc1]

Dodecyl ((4-nitrophenoxy)(phenoxy)phosphoryl)-L-alaninate. Dodecyl (tert-butoxycarbonyl)-L-alaninate (600 mg, 1.67 mmol) was dissolved in anhydrous 4 M HCl in dioxane (10 mL). The reaction was stirred for 1 hr. The reaction was concentrated under reduced pressure.

Phenyl dichlorophosphate (275 uL, 1.85 mmol) was dissolved in anhydrous DCM (15 mL) and stirred in an ice bath under atmospheric nitrogen. Above de-Boc material was dissolved in anhydrous DCM (5 mL) and added to the reaction dropwise. The reaction was stirred for 30 mins. Triethylamine (560 uL, 4 mmol) was added dropwise and then stirred for 60 mins. p-Nitrophenol (210 mg, 1.51 mmol) and triethylamine (281 uL, 2.01 mmol) were added. Ice bath was removed and the reaction mixture was stirred for 2 hrs at RT.

Reaction was diluted with DCM (40 mL) and washed with water (3×20 mL). Organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 8.22 (m, 2H), 7.45-7.29 (m, 4H), 7.28-7.15 (m, 3H), 4.20-4.02 (m, 3H), 3.88 (m, 1H), 1.60 (m, 2H), 1.41 (m, 3H), 1.25 (m, 18H), 0.88 (t, J=6.6 Hz, 3H). ³¹P NMR (162 MHz, Chloroform-d) δ −3.06 (s), −3.11 (s).

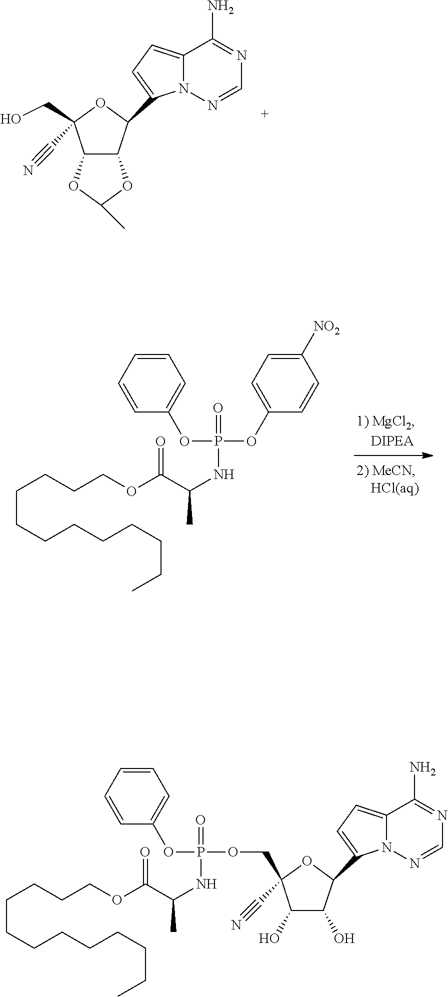

Dodecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.151 mmol) and dodecyl ((4-nitrophenoxy)(phenoxy)phosphoryl)-L-alaninate (89 mg, 0.166 mmol) were mixed and dissolved in anhydrous THF (5 mL). Magnesium chloride (72 mg, 0.755 mmol) was added in one portion and the reaction was stirred at 50° C. for 20 mins. DIPEA (131 uL, 0.755 mmol) was added and the reaction was stirred at 50° C. for 16 hrs.

Reaction was cooled to RT, diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (250 uL) was added dropwise. Reaction was stirred for 1 hr. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-3-5% methanol/DCM). Fractions having the desired product were combined and concentrated under reduced pressure and the resulting product was then dissolved in MeCN and water and freeze-dried to afford the product. ¹H NMR (400 MHz, Methanol-d4) δ 7.80 (m, 1H), 7.40-7.10 (m, 5H), 6.86 (m, 1H), 6.74 (m, 1H), 5.52 (m, 1H), 4.63 (m, 1H), 4.58-4.30 (m, 3H), 4.15-3.82 (m, 3H), 1.57 (m, 2H), 1.28 (m, 21H), 0.90 (t, J=6.8 Hz, 3H). ³¹P NMR (162 MHz, Methanol-d4) δ 3.26 (s). MS m/z=687.1 [M+1]; 685.3 [M−1].

### Example 192. hexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

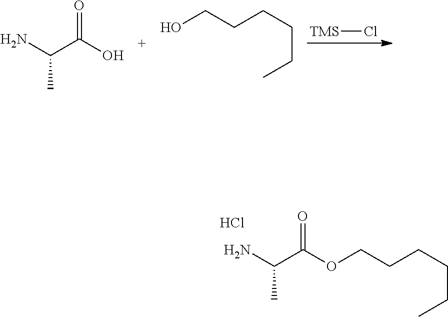

Hexyl L-alaninate hydrochloride. L-Alanine (4.45 g, 50 mmol) was mixed with 1-hexanol (30 mL). TMS-Cl (19.1 mL, 150 mmol) was added dropwise and the reaction was stirred at RT for 16 hrs.

More 1-hexanol (10 mL) and TMS-Cl (5 mL) were added. Reaction mixture was heated to 80° C. and stirred for 20 hrs.

Reaction was concentrated under reduced pressure, dried under high vacuum to afford the intermediate as hydrochloride salt. ¹H NMR (400 MHz, Chloroform-d) δ 8.77 (s, 3H), 4.20 (m, 3H), 1.71 (m, 5H), 1.47-1.19 (m, 6H), 1.01-0.78 (m, 3H).

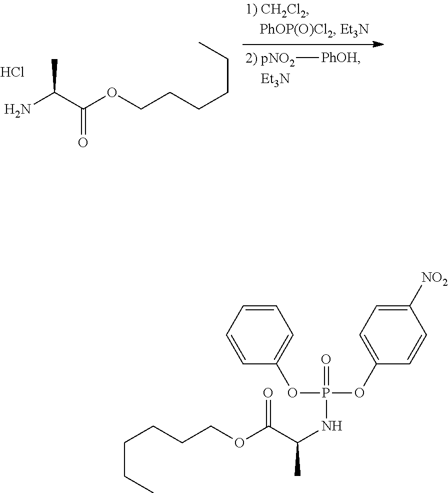

Hexyl ((4-nitrophenoxy)(phenoxy)phosphoryl)-L-alaninate. Phenyl dichlorophosphate (3.7 mL, 25 mmol) was dissolved in anhydrous DCM (50 mL) and stirred in an ice bath under atmospheric nitrogen. Hexyl L-alaninate hydrochloride (5.2 g, 25 mmol) was added in one portion. The reaction was stirred for 30 mins. Triethylamine (8.4 mL, 60 mmol) was added dropwise and then stirred for 60 mins. p-Nitrophenol (3.1 g, 22.5 mmol) and triethylamine (4.2 mL, 30 mmol) were added. Ice bath was removed and the reaction mixture was stirred for 16 hrs at RT. Reaction was diluted with DCM (100 mL) and washed with water (3×20 mL). Organic layer was dried over anhydrous sodium sulfate and concentrated under reduced pressure. The crude residue was purified via SiO₂ column chromatography (120 g SiO₂ Combiflash HP Gold Column, 0-20% ethyl acetate/hexanes). Fractions having the desired product were combined and concentrated under reduced pressure to afford the intermediate. ¹H NMR (400 MHz, Chloroform-d) δ 8.25 (d, J=9.1 Hz, 2H), 7.51-7.32 (m, 4H), 7.32-7.15 (m, 3H), 4.14 (m, 3H), 3.93 (m, 1H), 1.62 (m, 2H), 1.44 (m, 3H), 1.39-1.20 (m, 6H), 0.99-0.82 (m, 3H). ³¹P NMR (162 MHz, Chloroform-d) δ −3.03 (s), −3.08 (s).

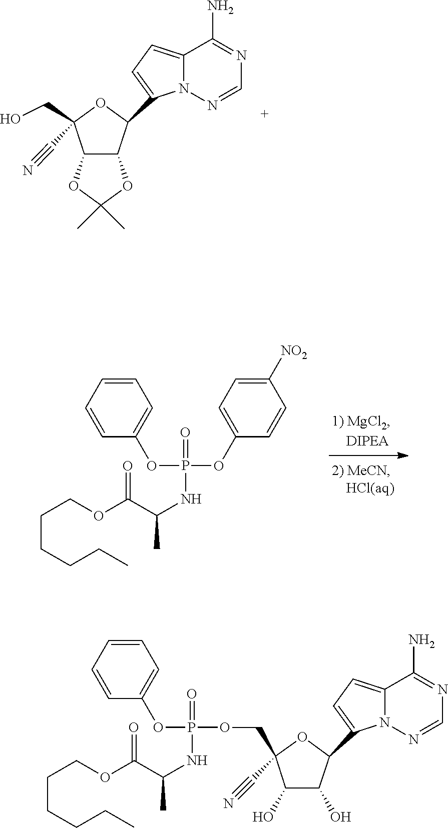 [SMILES: CC1(C)O[C@H]2[C@H](c3ccc4c(N)ncnn34)O[C@](C#N)(CO)[C@H]2O1 ; CCCCCCOC(=O)[C@H](C)NP(=O)(Oc1ccccc1)Oc1ccc([N+](=O)[O-])cc1 ; CCCCCCOC(=O)[C@H](C)NP(=O)(OC[C@@]1(C#N)O[C@@H](c2ccc3c(N)ncnn23)[C@H](O)[C@@H]1O)Oc1ccccc1]

Hexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.151 mmol) and hexyl ((4-nitrophenoxy)(phenoxy)phosphoryl)-L-alaninate (75 mg, 0.166 mmol) were mixed and dissolved in anhydrous THF (5 mL). Magnesium chloride (72 mg, 0.755 mmol) was added in one portion and the reaction was stirred for 5 mins. DIPEA (131 uL, 0.755 mmol) was added and the reaction was stirred at 50° C. for 36 hrs.

Reaction was cooled to RT, diluted with EtOAc (20 mL) and washed with water (5×20 mL) and with brine (10 mL). Organic layer was dried over anhydrous sodium sulfate and concentrated under reduced pressure which was then dissolved in MeCN (5 mL). 12 M HCl (aq) (250 uL) was added dropwise. Reaction was stirred for 1 hr. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-4-10% methanol/DCM). Fractions having the desired product were combined and concentrated under reduced pressure, then dissolved in MeCN and water and freeze-dried to afford the product. ¹H NMR (400 MHz, Methanol-d4) δ 7.80 (m, 1H), 7.42-7.09 (m, 5H), 6.86 (m, 1H), 6.74 (m, 1H), 5.57-5.44 (m, 1H), 4.64 (m, 1H), 4.58-4.28 (m, 3H), 4.17-3.81 (m, 3H), 1.64-1.48 (m, 2H), 1.38-1.19 (m, 9H), 0.94-0.82 (m, 3H). ³¹P NMR (162 MHz, Methanol-d4) δ 3.27 (s). MS m/z=603.0 [M+1]; 600.8 [M−1].

### Example 193. octadecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

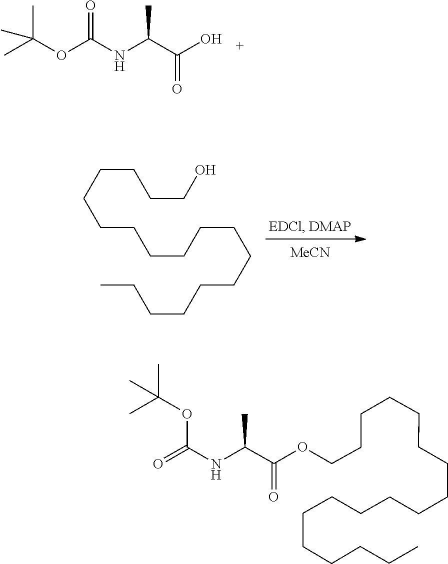 [SMILES: "MeCN" ; C[C@H](NC(=O)OC(C)(C)C)C(=O)O ; CCCCCCCCCCCCCCCCCCO ; CCCCCCCCCCCCCCCCCCOC(=O)[C@H](C)NC(=O)OC(C)(C)C]

Octadecyl (tert-butoxycarbonyl)-L-alaninate. Boc-L-Ala (5.67 g, 30 mmol) was dissolved in anhydrous acetonitrile (50 mL). EDCI (6.9 g, 36 mmol) was added in one portion and the reaction was stirred for 20 mins. 1-Octodecanol (9.74 g, 36 mmol) was added. DMAP (4 g, 33 mmol) was then added and the reaction was stirred for 2 hrs. More anhydrous acetonitrile (30 mL) was added and the reaction was stirred for 20 hrs. Anhydrous DMF (25 mL) was added and the reaction was stirred for 2 hrs. Reaction was diluted with EtOAc (100 mL) and washed with 5% aqueous citric acid solution (2×20 mL), followed with brine (20 mL). Organic layer was dried over anhydrous sodium sulfate and concentrated under reduced pressure. The crude residue was purified via SiO₂ column chromatography (80 g SiO₂ Combiflash HP Gold Column, 0-10% ethyl acetate/hexanes). Fractions having the desired product were combined and concentrated under reduced pressure to afford the intermediate. ¹H NMR (400 MHz, Chloroform-d) δ 5.04 (m, 1H), 4.39-4.21 (m, 1H), 4.12 (q, J=6.4 Hz, 2H), 1.62 (m, 2H), 1.44 (s, 9H), 1.38 (d, J=7.1 Hz, 3H), 1.25 (s, 30H), 0.87 (t, J=6.8 Hz, 3H).

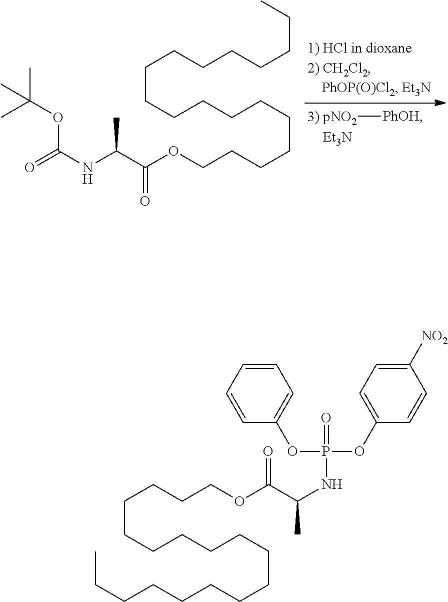

Octadecyl ((4-nitrophenoxy)(phenoxy)phosphoryl)-L-alaninate. octadecyl (tert-butoxycarbonyl)-L-alaninate (663 mg, 1.5 mmol) was dissolved in anhydrous 4 M HCl in dioxane (15 mL). The reaction was stirred for 2 hrs. The reaction was concentrated under reduced pressure to give solid which was mixed with anhydrous DCM (25 mL) and stirred in an ice bath under atmospheric nitrogen. Phenyl dichlorophosphate (223 uL, 1.5 mmol) was added to the reaction in one portion. The reaction was stirred for 15 mins. Triethylamine (500 uL, 3.6 mmol) was added dropwise and then stirred for 2 hrs. p-Nitrophenol (188 mg, 1.35 mmol) and triethylamine (251 uL, 1.8 mmol) were added. Ice bath was removed and the reaction mixture was stirred for 3 hrs at RT. Reaction was diluted with DCM (50 mL) and washed with water (3×20 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the intermediate. ¹H NMR (400 MHz, Chloroform-d) δ 8.23 (m, 2H), 7.49-7.30 (m, 4H), 7.30-7.12 (m, 3H), 4.23-3.98 (m, 3H), 3.88 (m, 1H), 1.69-1.52 (m, 2H), 1.41 (m, 3H), 1.25 (s, 30H), 0.88 (t, J=6.6 Hz, 3H). ³¹P NMR (162 MHz, Chloroform-d) δ −3.05 (s), −3.09 (s).

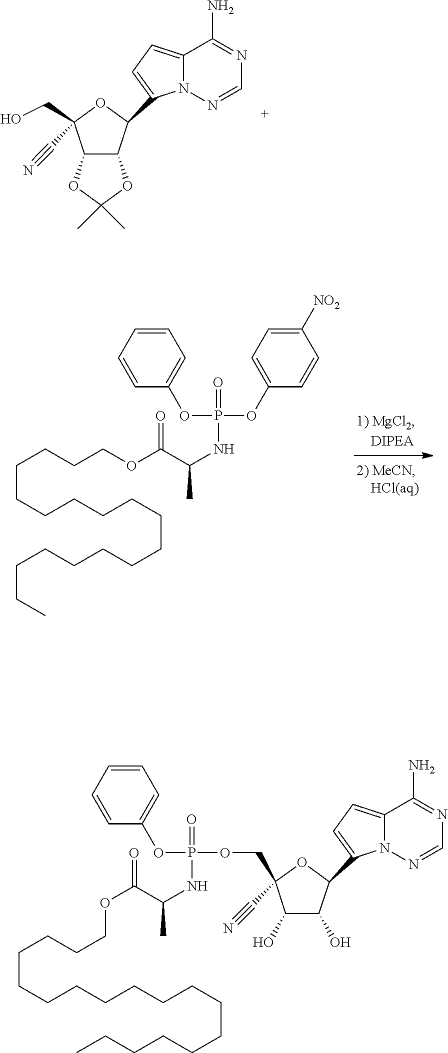 [SMILES: CC1(C)O[C@H]2[C@H](c3ccc4c(N)ncnn34)O[C@](C#N)(CO)[C@H]2O1 ; CCCCCCCCCCCCCCCCCCOC(=O)[C@H](C)NP(=O)(Oc1ccccc1)Oc1ccc([N+](=O)[O-])cc1 ; CCCCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

Octadecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.151 mmol) and octadecyl ((4-nitrophenoxy)(phenoxy)phosphoryl)-L-alaninate (103 mg, 0.166 mmol) were mixed and dissolved in anhydrous THF (3 mL). Magnesium chloride (72 mg, 0.755 mmol) was added in one portion and the reaction was stirred for 10 mins. DIPEA (131 uL, 0.755 mmol) was added and the reaction was stirred at 50° C. for 6 hrs.

Reaction was cooled to RT, diluted with EtOAc (20 mL) and washed with water (3×20 mL) and then with brine (10 mL). Organic layer was dried over anhydrous sodium sulfate and concentrated under reduced pressure which was then dissolved in MeCN (5 mL). 12 M HCl(aq) (250 uL) was added dropwise. Reaction was stirred for 1.5 hrs. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-4% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.85-7.72 (m, 1H), 7.40-7.08 (m, 5H), 6.88-6.79 (m, 1H), 6.79-6.66 (m, 1H), 5.55-5.43 (m, 2H), 4.61 (m, 1H), 4.55-4.26 (m, 3H), 4.14-3.79 (m, 3H), 1.55 (m, 2H), 1.27 (m, 33H), 0.89 (t, J=6.7 Hz, 3H). ³¹P NMR (162 MHz, Methanol-d4) δ 3.28 (s). MS m/z=771.0 [M+1]; 768.5 [M−1].

### Example 194. trans-4-(trifluoromethyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-D-alaninate

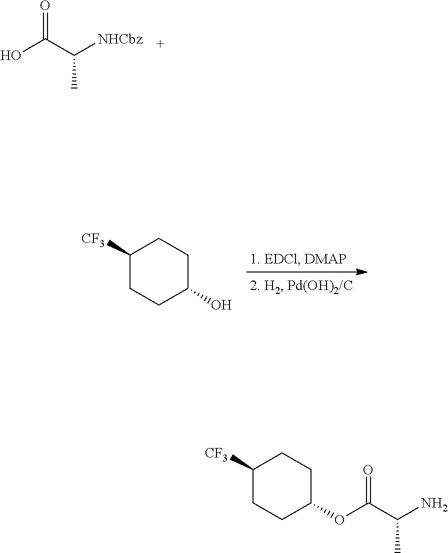

Trans-4-(trifluoromethyl)cyclohexyl D-alaninate. The intermediate was prepared from Cbz-D-alanine (990 mg, 4.03 mmol) and trans-4-(trifluoromethyl)cyclohexanol (1.0 g, 5.95 mmol) by the same method used for Intermediate 26. MS m/z 240=[M+H].

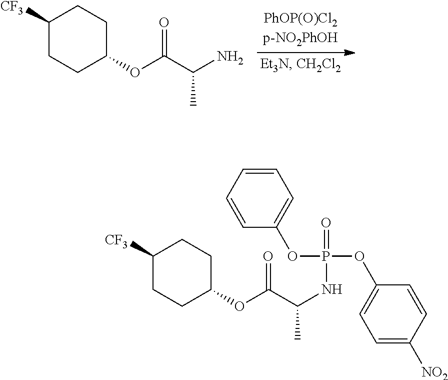

Trans-4-(trifluoromethyl)cyclohexyl ((4-nitrophenoxy)(phenoxy)phosphoryl)-D-alaninate. The intermediate was prepared as isomeric mixture from trans-4-(trifluoromethyl)cyclohexyl D-alaninate (0.95 g, 3.97 mmol) in a manner similar to that described for Intermediate 25. ¹H NMR (400 MHz, Chloroform-d) δ 8.21 (m, 2H), 7.48-7.29 (m, 4H), 7.22 (m, 3H), 4.67 (m, 1H), 4.06 (m, 2H), 2.01 (m, 5H), 1.52-1.20 (m, 7H). ³¹P NMR (162 MHz, Chloroform-d) δ −3.01, −3.06. ¹⁹F NMR (377 MHz, Chloroform-d) δ −73.89 (d, J=7.9 Hz). MS m/z=517 [M+H].

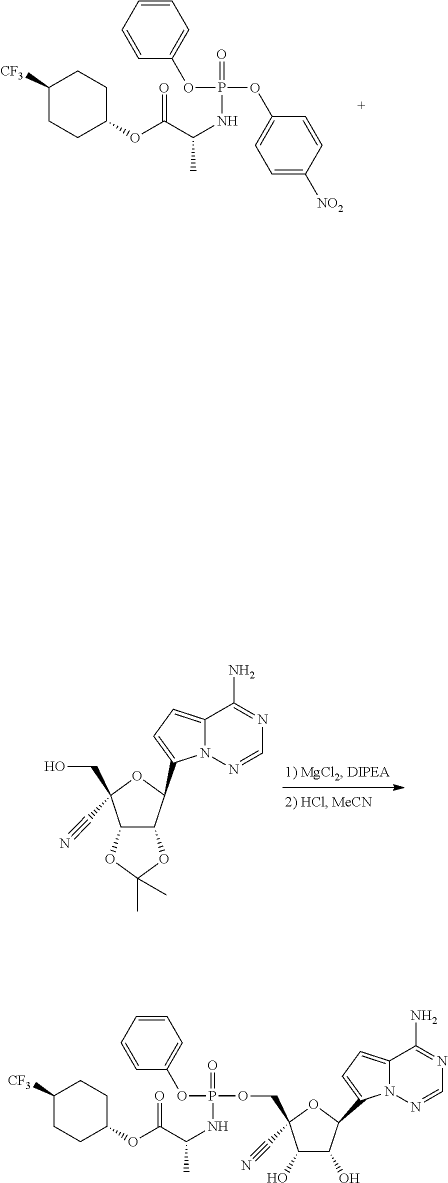 [SMILES: C[C@@H](NP(=O)(Oc1ccccc1)Oc1ccc([N+](=O)[O-])cc1)C(=O)O[C@H]1CC[C@H](C(F)(F)F)CC1 ; CC1(C)O[C@H]2[C@H](c3ccc4c(N)ncnn34)O[C@](C#N)(CO)[C@H]2O1 ; C[C@@H](NP(=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

Trans-4-(trifluoromethyl)cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-D-alaninate. The product was obtained from trans-4-(trifluoromethyl)cyclohexyl ((4-nitrophenoxy)(phenoxy)phosphoryl)-D-alaninate (117 mg, 0.23 mmol) and Intermediate 4 (50 mg, 0.15 mmol) in a manner similar to that described for Example 3. ¹H NMR (400 MHz, Methanol-d4) δ 7.81 (s, 0.58H), 7.78 (s, 0.42H), 7.31 (m, 2H), 7.23-7.12 (m, 3H), 6.88-6.83 (m, 1H), 6.76 (d, J=4.5 Hz, 0.58H), 6.72 (d, J=4.5 Hz, 0.42H), 5.50 (m, 1H), 4.70-4.54 (m, 2H), 4.52-4.42 (m, 2H), 4.35 (m, 1H), 3.86 (m, 1H), 1.99 (m, 5H), 1.50-1.27 (m, 4H), 1.24 (d, J=7.1 Hz, 1.26H), 1.20 (d, J=7.0 Hz, 1.74H). ³¹P NMR (162 MHz, Methanol-d4) δ 3.48, 3.00. ¹⁹F NMR (377 MHz, Methanol-d4) δ −75.39 (m). MS m/z=669 [M+1].

The product was separated via chiral preparatory HPLC (Chiralpak IA, 150×4.6 mm, Heptane 70% isopropanol 30%) to afford the diastereomers:

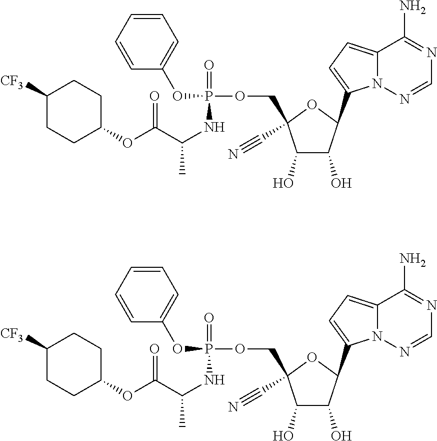 [SMILES: C[C@@H](N[P@@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1 ; C[C@@H](N[P@](=O)(OC[C@@]1(C#N)O[C@@H](c2ccc3c(N)ncnn23)[C@H](O)[C@@H]1O)Oc1ccccc1)C(=O)O[C@H]1CC[C@H](C(F)(F)F)CC1]

### Example 195. First Eluting Diastereomer

¹H NMR (400 MHz, Methanol-d4) δ 7.81 (s, 1H), 7.38-7.28 (m, 2H), 7.28-7.12 (m, 3H), 6.85 (d, J=4.5 Hz, 1H), 6.76 (d, J=4.6 Hz, 1H), 5.51 (d, J=5.2 Hz, 1H), 4.62 (m, 2H), 4.49 (d, J=5.6 Hz, 1H), 4.45 (dd, J=10.9, 5.6 Hz, 1H), 4.38 (dd, J=10.8, 5.7 Hz, 1H), 3.97-3.79 (m, 1H), 2.18-1.82 (m, 6H), 1.46-1.24 (m, 3H), 1.20 (dd, J=7.2, 1.2 Hz, 3H). ³¹P NMR (162 MHz, Methanol-d4) δ 3.48. ¹⁹F NMR (377 MHz, Methanol-d4) δ −75.42 (d, J=8.5 Hz).

### Example 196. Second Eluting Diastereomer

¹H NMR (400 MHz, Methanol-d4) δ 7.78 (s, 1H), 7.30 (t, J=7.8 Hz, 2H), 7.22-7.11 (m, 3H), 6.86 (d, J=4.5 Hz, 1H), 6.71 (d, J=4.5 Hz, 1H), 5.49 (d, J=5.1 Hz, 1H), 4.60 (dt, J=10.8, 5.7 Hz, 2H), 4.49-4.41 (m, 2H), 4.31 (dd, J=10.8, 5.1 Hz, 1H), 3.90-3.79 (m, 1H), 2.00 (td, J=34.4, 32.8, 9.9 Hz, 5H), 1.50-1.16 (m, 7H). ³¹P NMR (162 MHz, Methanol-d4) δ 2.97. ¹⁹F NMR (376 MHz, Methanol-d4) δ −75.42 (d, J=8.4 Hz).

### Example 197. 3-(hexadecyloxy)propyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl) methoxy)(phenoxy)phosphoryl)alaninate

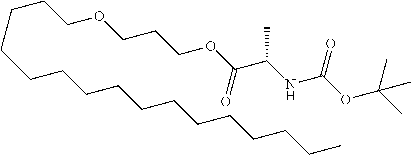 [SMILES: CCCCCCCCCCCCCCCCOCCCOC(=O)[C@H](C)NC(=O)OC(C)(C)C]

3-(Hexadecyloxy)propyl (tert-butoxycarbonyl)-L-alaninate. To a mixture of 3-(hexadecyloxy)propan-1-ol (1.85 g, 6.156 mmol), (tert-butoxycarbonyl)-L-alanine (1.864 g, 9.85 mmol) and triphenylphosphine (3.714 g, 14.16 mmol) in tetrahydrofuran (20 mL) at 0° C. was added diisopropyl azodicarboxylate (2.67 mL, 2.74 mmol). The reaction mixture was stirred at room temperature for 2 h and was concentrated under reduced pressure. The crude residue was purified by SiO₂ column chromatography (120 g SiO₂ Combiflash HP Gold Column, 0-20% ethyl acetate/hexanes as eluent) to afford the intermediate. ¹H NMR (400 MHz, Chloroform-d) δ 5.04 (d, J=5.1 Hz, 1H), 4.39-4.10 (m, 3H), 3.46 (t, J=6.2 Hz, 2H), 3.38 (t, J=6.7 Hz, 2H), 1.90 (p, J=6.3 Hz, 2H), 1.60-1.49 (m, 2H), 1.37 (d, J=7.2 Hz, 3H), 1.25 (s, 35H), 0.92-0.83 (m, 3H).

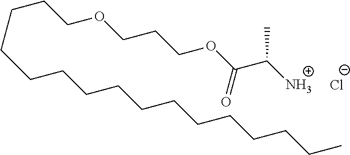 [SMILES: CCCCCCCCCCCCCCCCOCCCOC(=O)[C@H](C)[NH3+] ; [Cl-]]

(S)-1-(3-(hexadecyloxy)propoxy)-1-oxopropan-2-aminium chloride. The intermediate was prepared in a manner similar to that described for Intermediate 35. ¹H NMR (400 MHz, DMSO-d6) δ 8.45 (s, 3H), 4.32-3.85 (m, 3H), 3.40 (d, J=6.3 Hz, 2H), 1.81 (q, J=6.3 Hz, 2H), 1.53-1.32 (m, 5H), 1.22 (s, 26H), 0.83 (t, J=6.4 Hz, 3H).

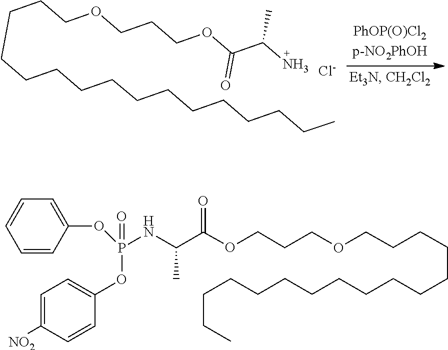

3-(hexadecyloxy)propyl) ((4-nitrophenoxy)(phenoxy)phosphoryl)-L-alaninate. The intermediate was prepared in a manner similar to that described for Intermediate 35. ¹H NMR (400 MHz, Acetonitrile-d3) δ 8.26 (dd, J=9.2, 2.0 Hz, 2H), 7.52-7.34 (m, 4H), 7.34-7.20 (m, 3H), 4.76-4.57 (m, 1H), 4.19-4.00 (m, 3H), 3.38 (dt, J=28.0, 6.4 Hz, 4H), 1.79 (td, J=6.3, 1.2 Hz, 2H), 1.50 (t, J=6.6 Hz, 2H), 1.38-1.23 (m, 27H), 0.95-0.81 (m, 3H). ³¹P NMR (162 MHz, Acetonitrile-d3) δ −2.16, −2.27. MS m/z=649.05 [M+1].

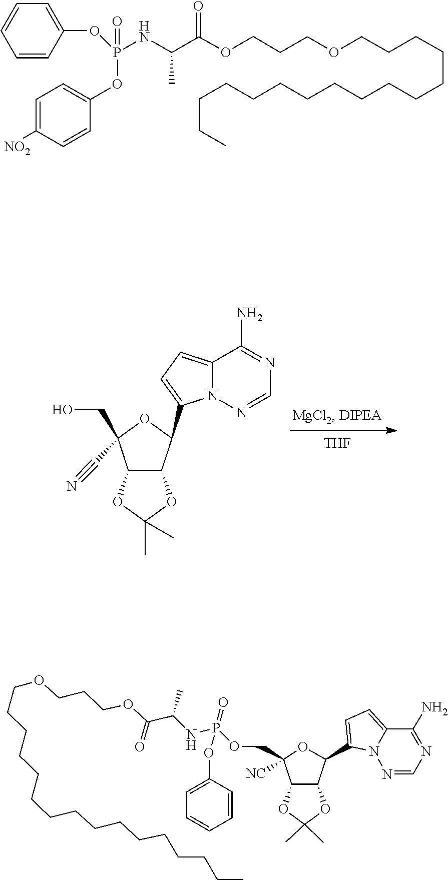

3-(Hexadecyloxy)propyl ((((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl) methoxy)(phenoxy)phosphoryl)alaninate. To a mixture of Intermediate 4 (50.0 mg, 0.151 mmol), 3-(hexadecyloxy)propyl) ((4-nitrophenoxy)(phenoxy)phosphoryl)-L-alaninate (187 mg, 0.288 mmol), and magnesium chloride (71.84 mg, 0.755 mmol) was added tetrahydrofuran (2 mL) at room temperature followed by the addition of N,N-Diisopropylethylamine (0.131 mL, 0.755 mmol). The resulting mixture was stirred at 50° C. for 3 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified via SiO₂ column chromatography (40 g SiO₂ Combiflash HP Gold Column, 30% ethyl acetate/hexanes-100% ethyl acetate as eluent) to afford the intermediate. ¹H NMR (400 MHz, Acetonitrile-d3) δ 7.89 (s, 1H), 7.42-7.28 (m, 2H), 7.28-7.11 (m, 3H), 6.77 (ddd, J=14.5, 4.5, 2.8 Hz, 2H), 6.28 (s, 2H), 5.67 (t, J=2.8 Hz, 1H), 5.29 (dd, J=6.6, 3.4 Hz, 1H), 5.09 (dd, J=6.6, 3.9 Hz, 1H), 4.48-4.20 (m, 3H), 4.18-4.02 (m, 2H), 3.93 (td, J=9.6, 7.0 Hz, 1H), 3.37 (dt, J=21.0, 6.3 Hz, 4H), 1.80 (dt, J=10.5, 6.3 Hz, 2H), 1.71 (d, J=2.9 Hz, 3H), 1.50 (t, J=6.7 Hz, 2H), 1.38 (d, J=4.2 Hz, 3H), 1.28 (s, 26H), 0.90 (t, J=6.6 Hz, 3H). ³¹P NMR (162 MHz, Acetonitrile-d3) δ 2.4, −2.38. MS m/z=842.07 [M+1].

### Example 198. 3-(hexadecyloxy)propyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

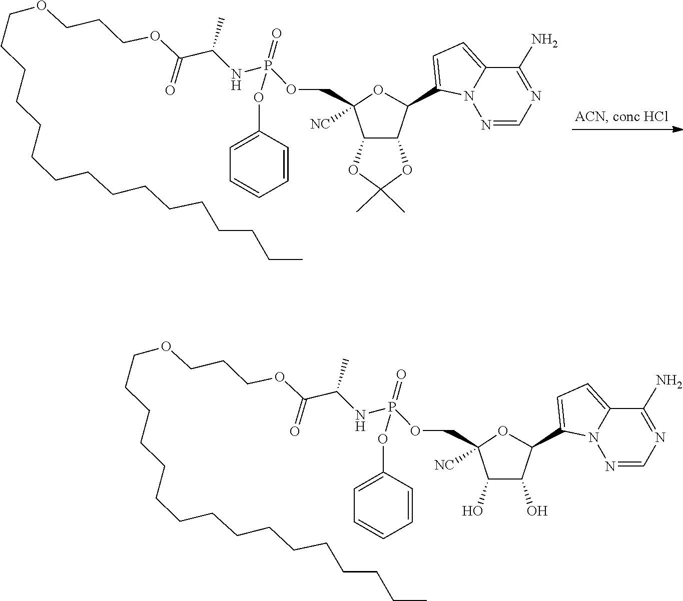

To a mixture of Example 197 (0.09 g, 0.107 mmol) in acetonitrile (2 mL) at 0° C. was added concentrated hydrochloric acid (0.1 mL, 2.743 mmol) and the reaction mixture was stirred at room temperature for 1 h. After 1 h the reaction mixture was cooled in an ice bath and was diluted with water. Neutralized the solution with 2 N sodium hydroxide and extracted with dichloromethane. Organic layer was separated, dried over sodium sulfate, filtered and concentrated. The residue obtained was purified by SiO₂ column chromatography (40 g SiO₂ Combiflash HP Gold Column, 100% Dichloromethane-10% Methanol in dichloromethane) to afford the product. ¹H NMR (400 MHz, DMSO-d6) δ 7.87 (m, 3H), 7.31 (dt, J=13.4, 7.9 Hz, 2H), 7.23-7.08 (m, 3H), 6.89 (dd, J=4.5, 1.9 Hz, 1H), 6.73 (dd, J=4.5, 2.2 Hz, 1H), 6.11 (ddd, J=13.3, 9.9, 3.5 Hz, 1H), 5.37 (t, J=5.7 Hz, 2H), 4.45 (t, J=5.7 Hz, 1H), 4.27 (td, J=11.4, 5.6 Hz, 2H), 4.15 (ddd, J=19.3, 10.9, 4.9 Hz, 1H), 4.09-3.91 (m, 2H), 3.86-3.72 (m, 1H), 3.39-3.18 (m, 4H), 1.70 (dq, J=12.7, 6.3 Hz, 2H), 1.41 (t, J=6.6 Hz, 2H), 1.28-1.10 (m, 26H), 0.90-0.69 (m, 3H). ³¹P NMR (162 MHz, DMSO-d6) δ 3.30, −3.24. MS m/z=801.78 [M+1].

### Example 199. hexadecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

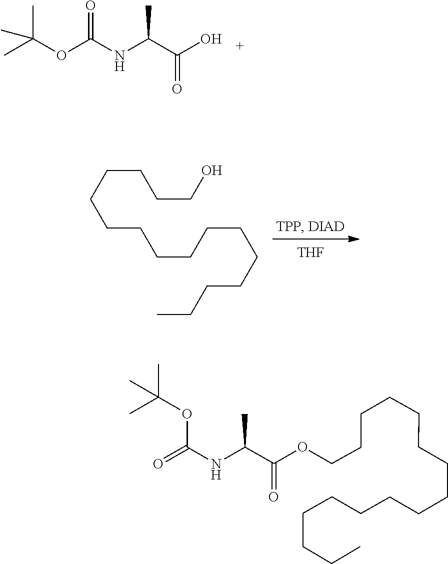 [SMILES: C[C@H](NC(=O)OC(C)(C)C)C(=O)O ; CCCCCCCCCCCCCCCCO ; CCCCCCCCCCCCCCCCOC(=O)[C@H](C)NC(=O)OC(C)(C)C ; "THF"]

Hexadecyl (tert-butoxycarbonyl)-L-alaninate. Boc-L-Ala (1.89 g, 10 mmol) was dissolved in anhydrous THF (50 mL). 1-Hexadecanol (3.6 g, 15 mmol) and triphenylphosphine (5.8 g, 22 mmol) were added. DIAD (3.9 mL, 20 mmol) was added dropwise. Reaction was stirred for 20 hrs.

Reaction was diluted with EtOAc (50 mL) and washed with saturated aqueous sodium bicarbonate solution (20 mL) and followed with brine (20 mL). Organic layer was dried over anhydrous sodium sulfate and concentrated under reduced pressure. The crude residue was purified via SiO₂ column chromatography (80 g SiO₂ Combiflash HP Gold Column, 0-10-20% ethyl acetate/hexanes). Fractions having the desired product were combined and concentrated under reduced pressure to afford the intermediate. ¹H NMR (400 MHz, Chloroform-d) δ 5.11-4.83 (m, 1H), 4.28 (m, 1H), 4.19-4.03 (m, 2H), 1.68-1.48 (m, 2H), 1.42 (s, 9H), 1.36 (d, J=7.2 Hz, 3H), 1.23 (m, 26H), 0.86 (t, J=6.7 Hz, 3H).

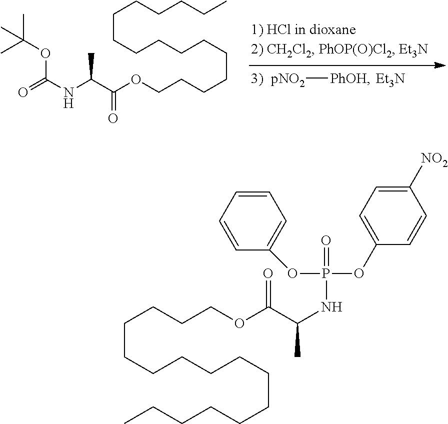

Hexadecyl ((4-nitrophenoxy)(phenoxy)phosphoryl)-L-alaninate. Hexadecyl (tert-butoxycarbonyl)-L-alaninate (3 g, 7.25 mmol) was dissolved in anhydrous 4 M HCl in dioxane (40 mL). The reaction was stirred for 16 hrs. The reaction was concentrated under reduced pressure to give solid which was mixed with anhydrous DCM (40 mL) and stirred in an ice bath under atmospheric nitrogen. Phenyl dichlorophosphate (1.08 mL, 7.25 mmol) was added to the reaction in one portion. The reaction was stirred for 15 mins. Triethylamine (2.4 mL, 17.4 mmol) was added dropwise and then stirred for 2 hrs. p-Nitrophenol (908 mg, 6.53 mmol) and triethylamine (1.21 mL, 8.7 mmol) were added. Ice bath was removed and the reaction mixture was stirred for 3 hrs at RT. Reaction was diluted with DCM (60 mL) and washed with water (5×20 mL). Organic layer was dried over anhydrous sodium sulfate and concentrated under reduced pressure. The crude residue was purified via SiO₂ column chromatography (40 g SiO₂ Combiflash HP Gold Column, 0-30% ethyl acetate/hexanes). Fractions with the desired product were combined and concentrated under reduced pressure to afford the intermediate. ¹H NMR (400 MHz, Chloroform-d) δ 8.22 (m, 2H), 7.47-7.29 (m, 4H), 7.29-7.09 (m, 3H), 4.11 (m, 3H), 3.87 (m, 1H), 1.60 (m, 2H), 1.41 (m, 3H), 1.25 (m, 26H), 0.87 (t, J=6.6 Hz, 3H). ³¹P NMR (162 MHz, Chloroform-d) δ −3.07 (s), −3.11 (s).

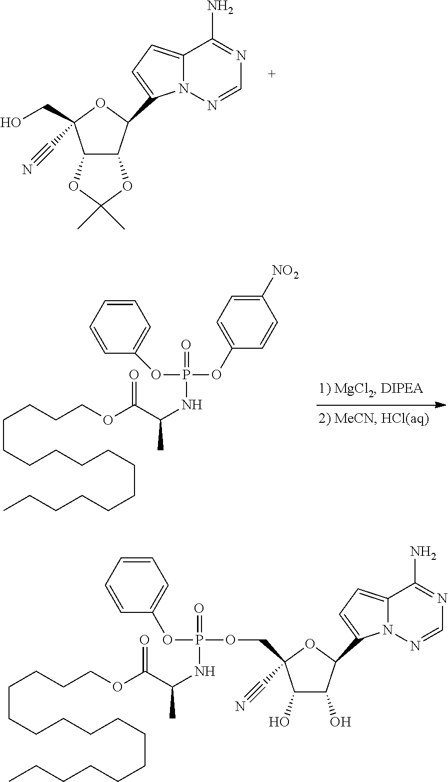 [SMILES: CC1(C)O[C@H]2[C@H](c3ccc4c(N)ncnn34)O[C@](C#N)(CO)[C@H]2O1 ; CCCCCCCCCCCCCCCCOC(=O)[C@H](C)NP(=O)(Oc1ccccc1)Oc1ccc([N+](=O)[O-])cc1 ; CCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

Hexadecyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (50 mg, 0.151 mmol) and hexadecyl ((4-nitrophenoxy)(phenoxy)phosphoryl)-L-alaninate (98 mg, 0.166 mmol) were mixed and dissolved in anhydrous THF (5 mL). Magnesium chloride (72 mg, 0.755 mmol) was added in one portion and the reaction was stirred for 20 mins. DIPEA (131 uL, 0.755 mmol) was added and the reaction was stirred at 50° C. for 6 hrs.

Reaction was cooled to RT, diluted with EtOAc (20 mL) and washed with water (5×20 mL) and then with brine (10 mL). Organic layer was dried over anhydrous sodium sulfate and concentrated under reduced pressure which was then dissolved in MeCN (5 mL). 12 M HCl(aq) (300 uL) was added dropwise. Reaction was stirred for 2 hrs. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-5-10% methanol/DCM). Fractions having the desired product were combined and concentrated under reduced pressure to give oil which was then dissolved in MeCN and water and freeze-dried to afford the product. ¹H NMR (400 MHz, Methanol-d4) δ 7.78 (m, 1H), 7.38-7.08 (m, 5H), 6.84 (m, 1H), 6.72 (m, 1H), 5.51 (m, 1H), 4.62 (m, 1H), 4.56-4.28 (m, 3H), 4.14-3.81 (m, 3H), 1.55 (m, 2H), 1.26 (m, 26H), 0.95-0.81 (m, 3H). ³¹P NMR (162 MHz, Methanol-d4) δ 3.25 (s). MS m/z=743.2 [M+1]; 741.0 [M−1].

### Example 200. 2-ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-(neopentyloxy)-1-oxopropan-2-yl)amino)phosphoryl)-L-alaninate

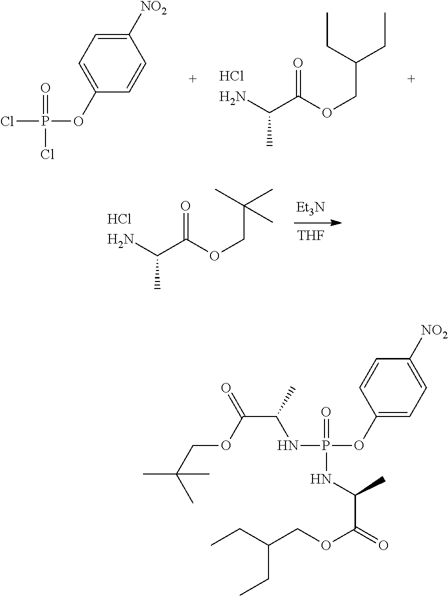

2-Ethylbutyl ((((S)-1-(neopentyloxy)-1-oxopropan-2-yl)amino)(4-nitrophenoxy)phosphoryl)-L-alaninate. 4-Nitrophenyl dichlorophosphate (256 mg, 1 mmol) was dissolved in anhydrous THF (20 mL) and stirred under atmospheric nitrogen in an ice bath. L-alanine 2-ethylbutyl ester hydrochloride (210 mg, 1 mmol) was added in one portion. Triethylamine (293 uL, 2.1 mmol) was added dropwise and the reaction was stirred for 1 hr. L-alanine neopentyl ester hydrochloride (196 mg, 1 mmol) was added in one portion. Triethylamine (293 uL, 2.1 mmol) was added dropwise and the reaction was stirred for 16 hrs at RT.

Reaction was diluted with EtOAc (20 mL) and washed with saturated sodium bicarbonate solution (2×10 mL) and then with brine (10 mL). Organic layer was dried over anhydrous sodium sulfate and concentrated under reduced pressure. The crude residue was purified via SiO₂ column chromatography (40 g SiO₂ Combiflash HP Gold Column, 0-50% ethyl acetate/hexanes). Fractions having the desired product were combined and concentrated under reduced pressure to afford the intermediate. ¹H NMR (400 MHz, Chloroform-d) δ 8.21 (d, J=8.8 Hz, 2H), 7.38 (d, J=8.8 Hz, 2H), 4.05 (m, 4H), 3.93-3.72 (m, 2H), 3.60 (m, 2H), 1.52 (m, 1H), 1.47-1.27 (m, 10H), 0.99-0.80 (m, 15H). ³¹P NMR (162 MHz, Chloroform-d) δ 7.98 (s).

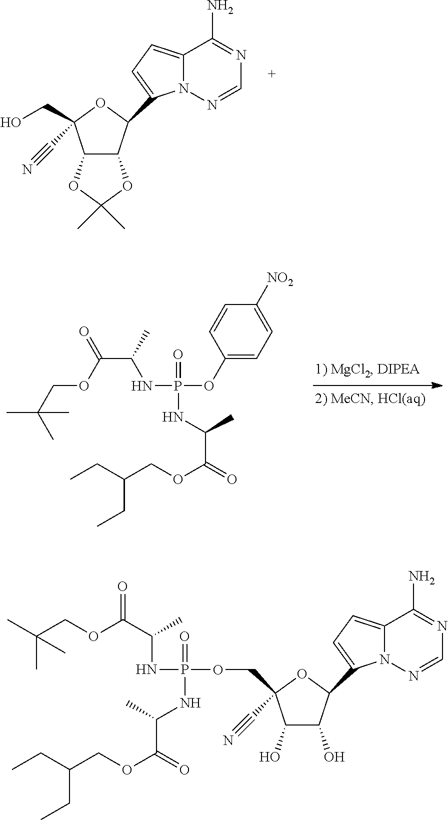 [SMILES: CC1(C)O[C@H]2[C@H](c3ccc4c(N)ncnn34)O[C@](C#N)(CO)[C@H]2O1 ; CCC(CC)COC(=O)[C@H](C)NP(=O)(N[C@@H](C)C(=O)OCC(C)(C)C)Oc1ccc([N+](=O)[O-])cc1 ; CCC(CC)COC(=O)[C@H](C)NP(=O)(N[C@@H](C)C(=O)OCC(C)(C)C)OC[C@@]1(C#N)O[C@@H](c2ccc3c(N)ncnn23)[C@H](O)[C@@H]1O]

2-Ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-(neopentyloxy)-1-oxopropan-2-yl)amino)phosphoryl)-L-alaninate. Intermediate 4 (50 mg, 0.151 mmol) and 2-ethylbutyl ((((S)-1-(neopentyloxy)-1-oxopropan-2-yl)amino)(4-nitrophenoxy)phosphoryl)-L-alaninate (86 mg, 0.166 mmol) were mixed and dissolved in anhydrous THF (5 mL). Magnesium chloride (72 mg, 0.755 mmol) was added in one portion and the reaction was stirred for 10 mins. DIPEA (66 uL, 0.378 mmol) was added and the reaction was stirred at RT for 20 hrs.

Reaction was diluted with EtOAc (20 mL) and washed with water (5×20 mL) and then with brine (10 mL). Organic layer was dried over anhydrous sodium sulfate and concentrated under reduced pressure which was then dissolved in MeCN (5 mL). 12 M HCl (aq) (250 uL) was added dropwise. Reaction was stirred for 1 hr. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-10% methanol/DCM). Fractions having the desired product were combined and concentrated under reduced pressure to give oil which was then dissolved in MeCN and water and freeze-dried to afford the product. ¹H NMR (400 MHz, Methanol-d4) δ 8.07 (s, 1H), 7.39 (d, J=4.7 Hz, 1H), 7.03 (d, J=4.7 Hz, 1H), 5.54 (d, J=5.2 Hz, 1H), 4.54 (t, J=5.4 Hz, 1H), 4.43 (d, J=5.5 Hz, 1H), 4.36-4.18 (m, 2H), 4.14-3.62 (m, 6H), 1.50 (m, 1H), 1.35 (m, 10H), 1.00-0.83 (m, 15H). ³¹P NMR (162 MHz, Methanol-d4) δ 13.57 (s). MS m/z=668.0 [M+1]; 665.9 [M−1].

### Example 201. cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(2,2,2-trifluoroethoxy)phosphoryl)-L-alaninate

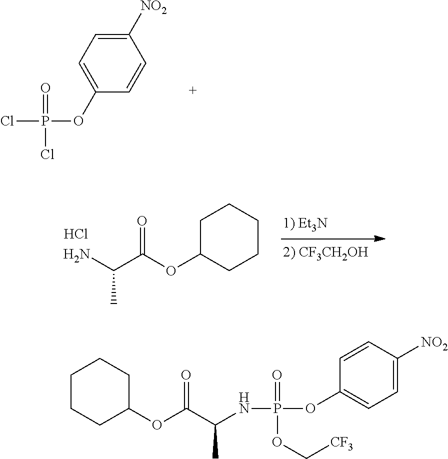

Cyclohexyl ((4-nitrophenoxy)(2,2,2-trifluoroethoxy)phosphoryl)-L-alaninate. 4-Nitrophenyl dichlorophosphate (256 mg, 1 mmol) was mixed with anhydrous DCM (10 mL) and stirred under atmospheric nitrogen in an ice bath. L-alanine cyclohexyl ester hydrochloride (208 mg, 1 mmol) was added in one portion and the reaction was stirred for 15 mins. Triethylamine (334 uL, 2.4 mmol) was added dropwise and the reaction was stirred for 1 hr. 2,2,2-trifluoroethanol (72 uL, 1 mmol) was added in one portion. Triethylamine (167 uL, 1.2 mmol) was added dropwise and the reaction was stirred for 3 hrs at RT.

Reaction was diluted with DCM (20 mL) and washed with water (2×20 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the product. ¹H NMR (400 MHz, Chloroform-d) δ 8.30-8.19 (m, 2H), 7.45-7.33 (m, 2H), 4.79 (m, 1H), 4.52-4.27 (m, 2H), 4.08-3.94 (m, 1H), 3.88-3.71 (m, 1H), 1.91-1.64 (m, 4H), 1.55-1.22 (m, 9H). ¹⁹F NMR (376 MHz, Chloroform-d) δ −75.56 (t, J=8.3 Hz). ³¹P NMR (162 MHz, Chloroform-d) δ 2.11 (s), 2.03 (s). MS m/z=454.7 [M+1]; 453.1 [M−1].

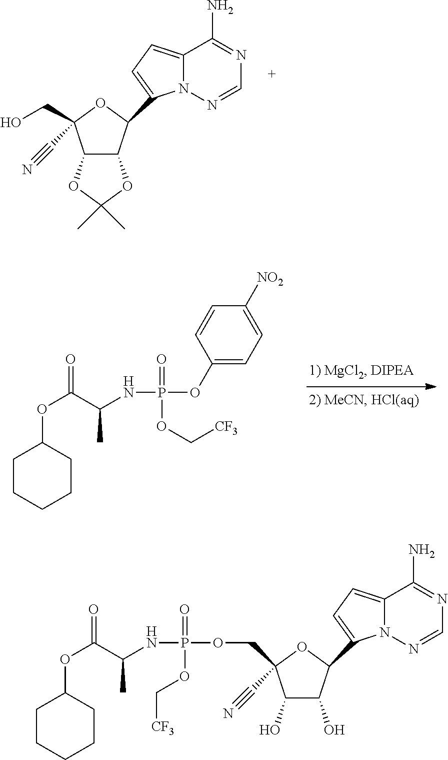 [SMILES: CC1(C)O[C@H]2[C@H](c3ccc4c(N)ncnn34)O[C@](C#N)(CO)[C@H]2O1 ; C[C@H](NP(=O)(OCC(F)(F)F)Oc1ccc([N+](=O)[O-])cc1)C(=O)OC1CCCCC1 ; C[C@H](NP(=O)(OCC(F)(F)F)OC[C@@]1(C#N)O[C@@H](c2ccc3c(N)ncnn23)[C@H](O)[C@@H]1O)C(=O)OC1CCCCC1]

Cyclohexyl ((((2R,3S,4R,5S)-5-(4-aminopyrrolo[2,1-f][1,2,4]triazin-7-yl)-2-cyano 3,4-dihydroxytetrahydrofuran-2-yl)methoxy)(2,2,2-trifluoroethoxy)phosphoryl)-L-alaninate. Intermediate 4 (50 mg, 0.151 mmol) and cyclohexyl ((4-nitrophenoxy)(2,2,2-trifluoroethoxy)phosphoryl)-L-alaninate (75 mg, 0.166 mmol) were mixed and dissolved in anhydrous THF (5 mL). Magnesium chloride (72 mg, 0.755 mmol) was added in one portion and the reaction was stirred for 15 mins. DIPEA (131 uL, 0.755 mmol) was added and the reaction was stirred at 50° C. for 16 hrs.

Reaction was cooled to RT, diluted with EtOAc (20 mL) and washed with water (5×20 mL) and then with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 2 hrs. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-5-10% methanol/DCM). Fractions having the desired product were combined and concentrated under reduced pressure and then dissolved in MeCN and water and freeze-dried to afford the product. ¹H NMR (400 MHz, Methanol-d4) δ 7.80 (m, 1H), 6.85 (d, J=4.5 Hz, 1H), 6.75 (d, J=4.5 Hz, 1H), 5.51 (m, 1H), 4.76-4.59 (m, 2H), 4.54-4.25 (m, 5H), 3.80 (m, 1H), 1.86-1.62 (m, 4H), 1.57-1.26 (m, 9H). ¹⁹F NMR (376 MHz, Methanol-d4) δ −77.28 (t, J=8.3 Hz), −77.25 (t, J=8.3 Hz). ³¹P NMR (162 MHz, Methanol-d4) δ 7.87 (s), 7.77 (s). MS m/z=607.0 [M+1]; 605.0 [M−1].

### Example 202. cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(hydroxy)phosphoryl)-L-alaninate

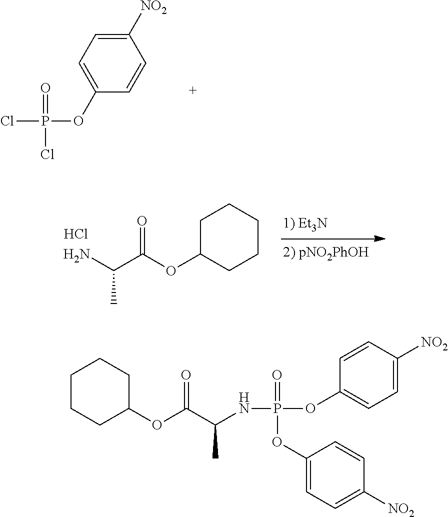

Cyclohexyl (bis(4-nitrophenoxy)phosphoryl)-L-alaninate. 4-Nitrophenyl dichlorophosphate (256 mg, 1 mmol) was mixed with anhydrous DCM (10 mL) and stirred under atmospheric nitrogen in an ice bath. L-alanine cyclohexyl ester hydrochloride (208 mg, 1 mmol) was added in one portion and the reaction was stirred for 15 mins. Triethylamine (334 uL, 2.4 mmol) was added dropwise and the reaction was stirred for 2 hrs. p-Nitrophenol (125 mg, 0.9 mmol) was added in one portion. Triethylamine (167 uL, 1.2 mmol) was added dropwise and the reaction was stirred for 3 hrs at RT.

Reaction was diluted with DCM (20 mL) and washed with water (4×20 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the intermediate. ¹H NMR (400 MHz, Chloroform-d) δ 8.30-8.19 (m, 4H), 7.46-7.35 (m, 4H), 4.76 (m, 1H), 4.19-4.04 (m, 1H), 3.99 (m, 1H), 1.86-1.62 (m, 4H), 1.54 (m, 2H), 1.47-1.29 (m, 7H). ³¹P NMR (162 MHz, Chloroform-d) δ −3.40 (s).

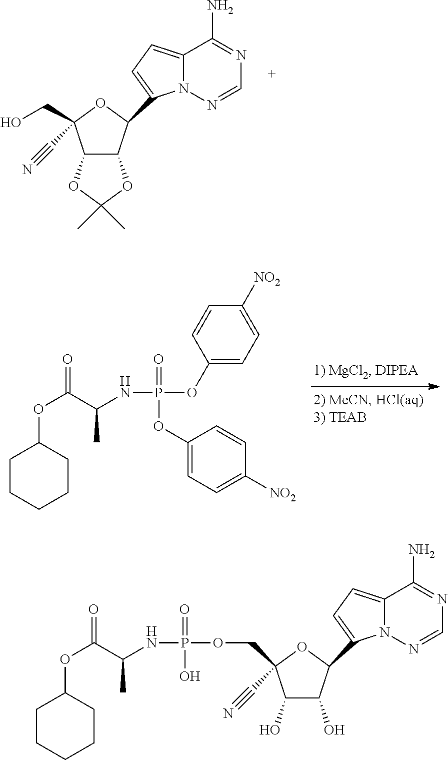 [SMILES: CC1(C)O[C@H]2[C@H](c3ccc4c(N)ncnn34)O[C@](C#N)(CO)[C@H]2O1 ; C[C@H](NP(=O)(Oc1ccc([N+](=O)[O-])cc1)Oc1ccc([N+](=O)[O-])cc1)C(=O)OC1CCCCC1 ; C[C@H](NP(=O)(O)OC[C@@]1(C#N)O[C@@H](c2ccc3c(N)ncnn23)[C@H](O)[C@@H]1O)C(=O)OC1CCCCC1]

Cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(hydroxy)phosphoryl)-L-alaninate. Intermediate 4 (50 mg, 0.151 mmol) and cyclohexyl (bis(4-nitrophenoxy)phosphoryl)-L-alaninate (82 mg, 0.166 mmol) were mixed and dissolved in anhydrous THF (5 mL). Magnesium chloride (72 mg, 0.755 mmol) was added in one portion and the reaction was stirred for 15 mins. DIPEA (131 uL, 0.755 mmol) was added and the reaction was stirred at RT for 5 hrs.

Reaction was diluted with EtOAc (20 mL) and washed with water (5×20 mL) and then with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 2 hrs. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-5-10% methanol/DCM). Fractions having the desired product were combined and concentrated under reduced pressure to give oil which was then dissolved in MeCN (3 mL) and stirred in an ice bath. 1 M triethyl ammonium bicarbonate solution (3 mL) was added and the reaction was stirred at RT for 3 hrs. Reaction was diluted with water (10 mL) and washed with EtOAc (2×10 mL). Aqueous was neutralized with 1 N HCl (aq) to give pH of 5-6. Material was then freeze-dried to give white powder. The crude residue was purified via prep HPLC C18 column (Phenominex Gemini 4 u 80 Å 150×30 mm column, 5-100% acetonitrile/water gradient with TEAB as modifier) to afford the product as a triethylammonium salt. ¹H NMR (400 MHz, Methanol-d4) δ 7.81 (s, 1H), 6.88-6.80 (m, 2H), 5.52 (d, J=5.8 Hz, 1H), 4.69 (td, J=8.8, 4.2 Hz, 1H), 4.57 (t, J=5.6 Hz, 1H), 4.48 (d, J=5.4 Hz, 1H), 4.12-3.98 (m, 2H), 3.90-3.77 (m, 1H), 3.06 (m, 6H), 1.84-1.74 (m, 2H), 1.74-1.63 (m, 2H), 1.55-1.19 (m, 18H). ³¹P NMR (162 MHz, Methanol-d4) δ 5.42 (s). MS m/z=525.1 [M+1]; 523.3 [M−1].

### Example 203. cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(naphthalen-1-yloxy)phosphoryl)-L-alaninate

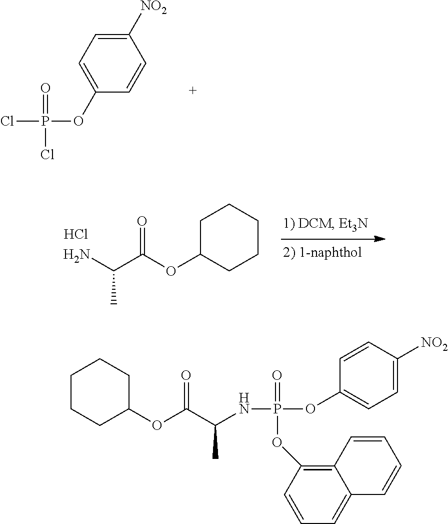

Cyclohexyl ((naphthalen-1-yloxy)(4-nitrophenoxy)phosphoryl)-L-alaninate. 4-Nitrophenyl dichlorophosphate (256 mg, 1 mmol) was mixed with anhydrous DCM (10 mL) and stirred under atmospheric nitrogen in an ice bath. L-alanine cyclohexyl ester hydrochloride (208 mg, 1 mmol) was added in one portion and the reaction was stirred for 20 mins. Triethylamine (334 uL, 2.4 mmol) was added dropwise and the reaction was stirred for 1 hr. 1-Naphthol (72 mg, 0.9 mmol) was added in one portion. Triethylamine (167 uL, 1.2 mmol) was added dropwise and the reaction was stirred for 3 hrs at RT.

Reaction was diluted with DCM (20 mL) and washed with water (2×20 mL). Organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions having the desired product were combined and concentrated under reduced pressure to afford the intermediate. ¹H NMR (400 MHz, Chloroform-d) δ 8.27-8.15 (m, 2H), 8.08-7.96 (m, 1H), 7.92-7.80 (m, 1H), 7.73-7.64 (m, 1H), 7.55 (m, 3H), 7.47-7.33 (m, 3H), 4.81-4.64 (m, 1H), 4.27-4.11 (m, 1H), 4.06-3.88 (m, 1H), 1.84-1.61 (m, 4H), 1.54 (m, 2H), 1.44-1.18 (m, 7H). ³¹P NMR (162 MHz, Chloroform-d) δ −2.77 (s), −2.81 (s).

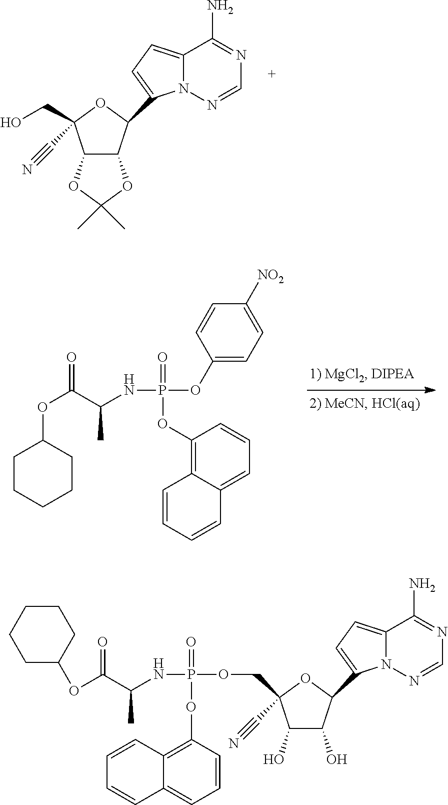 [SMILES: CC1(C)O[C@H]2[C@H](c3ccc4c(N)ncnn34)O[C@](C#N)(CO)[C@H]2O1 ; C[C@H](NP(=O)(Oc1ccc([N+](=O)[O-])cc1)Oc1cccc2ccccc12)C(=O)OC1CCCCC1 ; C[C@H](NP(=O)(OC[C@@]1(C#N)O[C@@H](c2ccc3c(N)ncnn23)[C@H](O)[C@@H]1O)Oc1cccc2ccccc12)C(=O)OC1CCCCC1]

Cyclohexyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(naphthalen-1-yloxy)phosphoryl)-L-alaninate. Intermediate 4 (50 mg, 0.151 mmol) and cyclohexyl ((naphthalen-1-yloxy)(4-nitrophenoxy)phosphoryl)-L-alaninate (83 mg, 0.166 mmol) were mixed and dissolved in anhydrous THF (5 mL). Magnesium chloride (72 mg, 0.755 mmol) was added in one portion and the reaction was stirred for 15 mins. DIPEA (131 uL, 0.755 mmol) was added and the reaction was stirred at 50° C. for 16 hrs.

Reaction was cooled to RT, diluted with EtOAc (20 mL) and washed with water (5×20 mL) and then with brine (10 mL). Organic layer was dried over anhydrous sodium sulfate and concentrated under reduced pressure which was then dissolved in MeCN (5 mL). 12 M HCl (aq) (300 uL) was added dropwise. Reaction was stirred for 2 hrs. Reaction was diluted with EtOAc (20 mL) and washed with saturated aqueous sodium bicarbonate solution (10 mL) and then with brine (10 mL). Organic layer was dried over anhydrous sodium sulfate and concentrated under reduced pressure. The crude residue was purified via SiO₂ column chromatography (4 g SiO₂ Combiflash HP Gold Column, 0-4% methanol/DCM). Fractions having the desired product were combined and concentrated under reduced pressure to give oil which was then dissolved in MeCN and water and freeze-dried to afford the product. ¹H NMR (400 MHz, Methanol-d4) δ 8.19-8.09 (m, 1H), 7.91-7.82 (m, 1H), 7.75 (m, 1H), 7.72-7.64 (m, 1H), 7.56-7.41 (m, 3H), 7.35 (m, 1H), 6.80 (m, 1H), 6.68 (m, 1H), 5.50 (m, 1H), 4.68-4.36 (m, 5H), 4.00-3.85 (m, 1H), 1.79-1.56 (m, 4H), 1.48-1.17 (m, 9H). ³¹P NMR (162 MHz, Methanol-d4) δ 3.75 (s), 3.62 (s). MS m/z=651.1 [M+1]; 649.0 [M−1].

### Example 204. 1-(2,2,2-trifluoroethyl)azepan-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate

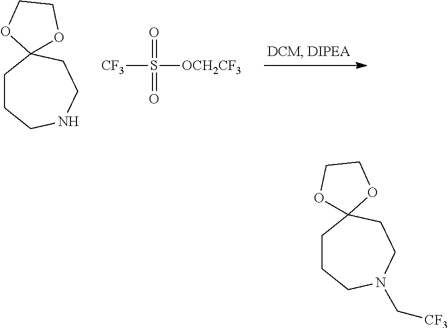

8-(2,2,2-Trifluoroethyl)1,4-dioxa-8-azaspiro[4.6]undecane. To an ice cold solution of 1,4-dioxa-8-azaspiro[4.6]undecane (4.73 g, 0.03 mol), Diisopropylamine (6.25 mL, 0.036 mol) in an anhydrous dichloromethane (15 mL) was added 2,2,2-Trifluoroethyl trifluoromethanesulfonate (4.74 mL, 0.033 mL). The resulting reaction mixture was stirred at room temperature overnight followed by purification via silica gel column chromatography (80 g column, 100% hexane to 40% ethyl acetate/hexane) to afford the intermediate. ¹H NMR (400 MHz, Chloroform-d) δ 3.90 (s, 4H), 3.12 (q, J=9.6 Hz, 2H), 2.88 (dt, J=25.5, 5.5 Hz, 4H), 1.95-1.81 (m, 4H), 1.69 (p, J=5.9 Hz, 2H). ¹⁹F NMR (376 MHz, Chloroform-d) δ −70.86 (t, J=9.7 Hz).

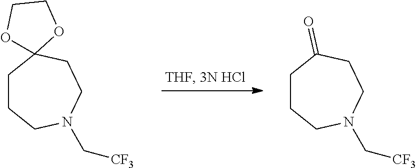 [SMILES: FC(F)(F)CN1CCCC2(CC1)OCCO2 ; O=C1CCCN(CC(F)(F)F)CC1]

1-(2,2,2-Trifluoroethyl)azepan-4-one. To a solution of 8-(2,2,2-trifluoroethyl)1,4-dioxa-8-azaspiro[4.6]undecane (5.197 g, 0.022 mol) in tetrahydrofuran (21 mL) was added 3 N hydrochloric acid (25 mL) and reaction mixture was stirred at room temperature overnight. Reaction mixture was cooled to 0° C. in an ice bath, diluted with water (10 mL) and neutralized with 3 N aqueous sodium hydroxide solution. Extraction was done with ethyl acetate. Organic layer was separated, dried over sodium sulfate, filtered and concentrated under reduced pressure. The residue obtained was purified via silica gel column chromatography (80 g column, 100% hexane to 70% ethyl acetate/hexane) to afford the intermediate. ¹H NMR (400 MHz, Chloroform-d) δ 3.16 (q, J=9.4 Hz, 2H), 3.00 (dt, J=13.6, 5.5 Hz, 4H), 2.66-2.50 (m, 4H), 1.87-1.75 (m, 2H). ¹⁹F NMR (376 MHz, Chloroform-d) δ −70.46 (t, J=9.3 Hz).

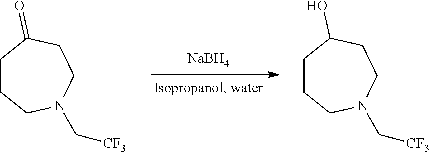 [SMILES: O=C1CCCN(CC(F)(F)F)CC1 ; "NaBH4" ; OC1CCCN(CC(F)(F)F)CC1]

1-(2,2,2-Trifluoroethyl)azepan-4-ol. To a solution of 1-(2,2,2-trifluoroethyl)azepan-4-one (3.8 g, 0.195 mol) in isopropanol (40 mL) was added sodium borohydride (0.737 g, 0.155 mol) and reaction mixture was stirred at room temperature for 3 h. Reaction mixture was diluted with water (20 mL) and stirred for 5 min. Extraction was done with ethyl acetate. Organic layer was separated, dried over sodium sulfate, filtered and concentrated under reduced pressure. The residue obtained was purified via silica gel column chromatography (80 g column, 100% hexane to 100% ethyl acetate/hexane) to afford the intermediate. ¹H NMR (400 MHz, DMSO-d6) δ 4.39 (d, J=4.1 Hz, 1H), 3.65 (tq, J=8.1, 4.0 Hz, 1H), 3.24 (qd, J=10.1, 1.0 Hz, 2H), 2.86-2.73 (m, 3H), 2.65 (ddd, J=13.8, 9.0, 2.9 Hz, 1H), 1.81-1.58 (m, 3H), 1.56-1.33 (m, 3H). ¹⁹F NMR (377 MHz, DMSO-d6) δ −70.01 (t, J=10.2 Hz).

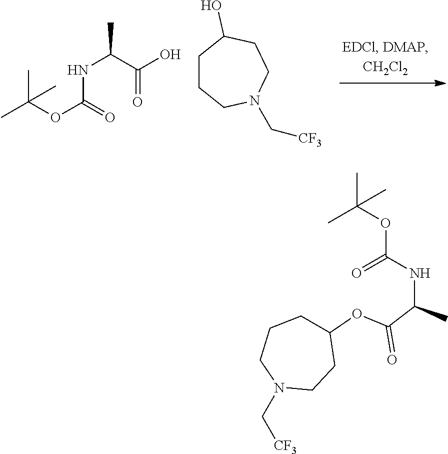

1-(2,2,2-Trifluoroethyl)azepan-4-yl (tert-butoxycarbonyl)-L-alaninate. The intermediate (was prepared in a manner similar to that described for Intermediate 12. ¹H NMR (400 MHz, DMSO-d6) δ 7.21 (d, J=7.2 Hz, 1H), 4.85 (tt, J=7.9, 4.0 Hz, 1H), 3.91 (p, J=7.3 Hz, 1H), 3.36-3.17 (m, 2H), 2.94-2.63 (m, 4H), 1.92-1.58 (m, 5H), 1.57-1.40 (m, 1H), 1.35 (s, 9H), 1.19 (d, J=7.3 Hz, 3H). ¹⁹F NMR (376 MHz, DMSO-d6) δ −70.06 (td, J=10.0, 3.5 Hz).

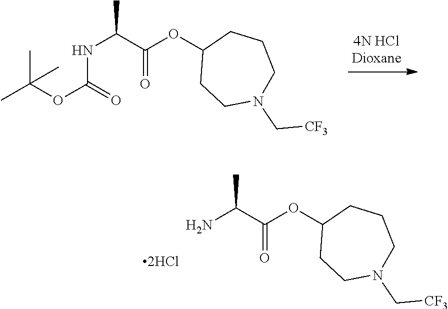 [SMILES: C[C@H](NC(=O)OC(C)(C)C)C(=O)OC1CCCN(CC(F)(F)F)CC1 ; C[C@H](N)C(=O)OC1CCCN(CC(F)(F)F)CC1]

1-(2,2,2-trifluoroethyl)azepan-4-yl L-alaninate dihydrochloride. The intermediate was prepared in a manner similar to that described for Intermediate 35. ¹H NMR (400 MHz, Methanol-d4) δ 5.23 (s, 1H), 4.16 (dt, J=15.0, 8.1 Hz, 2H), 3.76-3.36 (m, 4H), 2.42-1.77 (m, 6H), 1.55 (dd, J=7.2, 1.2 Hz, 3H).

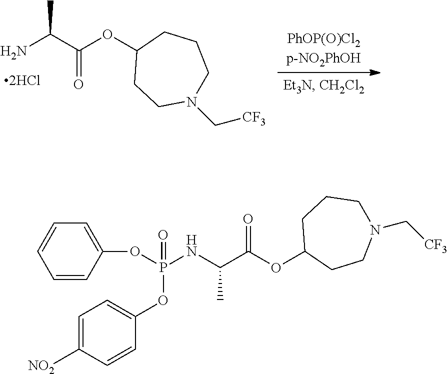 [SMILES: C[C@H](N)C(=O)OC1CCCN(CC(F)(F)F)CC1 ; C[C@H](NP(=O)(Oc1ccccc1)Oc1ccc([N+](=O)[O-])cc1)C(=O)OC1CCCN(CC(F)(F)F)CC1]

1-(2,2,2-Trifluoroethyl)azepan-4-yl ((4-nitrophenoxy)(phenoxy)phosphoryl)-L-alaninate. The intermediate was prepared in a manner similar to that described for Intermediate 35. ¹H NMR (400 MHz, Chloroform-d) δ 8.30-8.16 (m, 2H), 7.45-7.30 (m, 4H), 7.29-7.16 (m, 3H), 4.98 (dt, J=8.1, 4.0 Hz, 1H), 4.22-4.03 (m, 1H), 3.91 (d, J=11.2 Hz, 1H), 3.14 (q, J=9.4 Hz, 2H), 3.00-2.71 (m, 4H), 2.05-1.67 (m, 6H), 1.39 (dt, J=7.2, 1.8 Hz, 3H). ³¹P NMR (162 MHz, Chloroform-d) δ −3.05. MS m/z=546.12 [M+1].

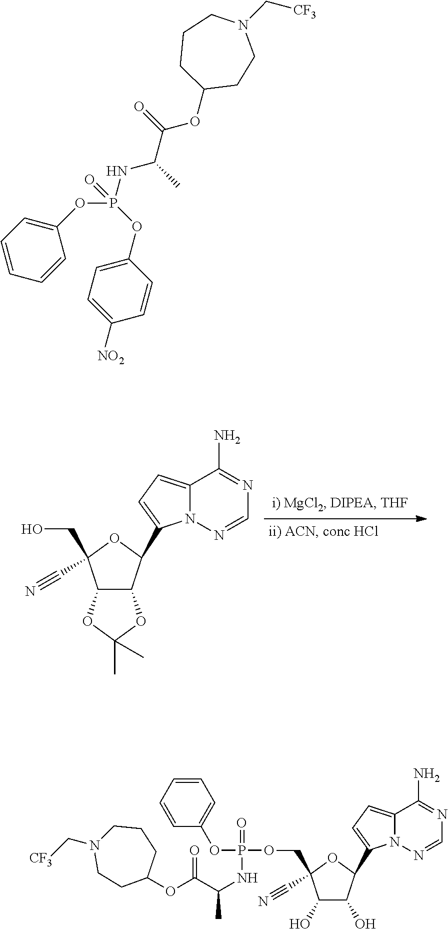 [SMILES: C[C@H](NP(=O)(Oc1ccccc1)Oc1ccc([N+](=O)[O-])cc1)C(=O)OC1CCCN(CC(F)(F)F)CC1 ; CC1(C)O[C@H]2[C@H](c3ccc4c(N)ncnn34)O[C@](C#N)(CO)[C@H]2O1 ; C[C@H](NP(=O)(OC[C@@]1(C#N)O[C@@H](c2ccc3c(N)ncnn23)[C@H](O)[C@@H]1O)Oc1ccccc1)C(=O)OC1CCCN(CC(F)(F)F)CC1]

1-(2,2,2-Trifluoroethyl)azepan-4-yl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)alaninate. To a mixture of Intermediate 4 (0.06 g, 0.181 mmol), 1-(2,2,2-trifluoroethyl)azepan-4-yl ((4-nitrophenoxy)(phenoxy)phosphoryl)-L-alaninate (0.119 g, 0.217 mmol), and magnesium chloride (0.028 g, 0.29 mmol) was added tetrahydrofuran (1.5 mL) at room temperature followed by the addition of N,N-Diisopropylethylamine (0.079 mL, 0.453 mmol). The resulting mixture was stirred at 50° C. for 3 h. The reaction mixture was then concentrated under reduced pressure and the residue obtained was diluted with saturated sodium chloride solution and dichloromethane. The layers were split and the organic layer was dried over anhydrous sodium sulfate, filtered and was concentrated under reduced pressure. The crude residue was purified by preparative HPLC (Phenominex Synergi 4 u Hydro-RR 80 Å 150×30 mm column, 15%-85% acetonitrile/water gradient in 30 min run). Pure material obtained was dissolved in an anhydrous acetonitrile (2 mL) and was cooled in an ice bath followed by the dropwise addition of concentrated hydrochloric acid (0.1 mL, 1.2 mmol). The reaction mixture was stirred at room temperature for 1 h. After 1 h the reaction mixture was cooled in an ice bath and was diluted with saturated sodium bicarbonate solution (1 mL). The resulting mixture was purified by preparative HPLC (Phenominex Synergi 4 u Hydro-RR 80 Å 150×30 mm column, 15%-85% acetonitrile/water gradient in 30 min run) to afford the intermediate. ¹H NMR (400 MHz, Acetonitrile-d3) δ 7.88 (d, J=4.4 Hz, 1H), 7.36 (q, J=8.5 Hz, 2H), 7.26-7.13 (m, 3H), 6.75 (d, J=1.7 Hz, 2H), 6.26 (s, 2H), 5.48 (t, J=4.6 Hz, 1H), 4.98-4.78 (m, 1H), 4.58 (s, 1H), 4.53-4.18 (m, 6H), 3.90 (dd, J=16.8, 9.5 Hz, 2H), 3.20 (qd, J=9.8, 5.6 Hz, 2H), 2.98-2.64 (m, 2H), 1.92-1.62 (m, 3H), 1.53 (s, 1H), 1.26 (t, J=7.7 Hz, 3H). ¹⁹F NMR (376 MHz, Acetonitrile-d3) δ −72.20 (td, J=10.0, 2.2 Hz). MS m/z=697.89 [M+1].

### Example 205. (2S,3R,4S,5R)-2-(4-Aminopyrrolo[2,1-f][1,2,4]triazin-7-yl)-5-cyano-5-((((S)—(((S)-1-(cyclohexyloxy)-1-oxopropan-2-yl)amino)(phenoxy)phosphoryl)oxy)methyl)-4-hydroxytetrahydrofuran-3-yl isobutyrate

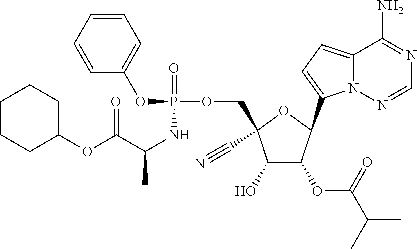 [SMILES: CC(C)C(=O)O[C@H]1[C@H](c2ccc3c(N)ncnn23)O[C@](C#N)(CO[P@@](=O)(N[C@@H](C)C(=O)OC2CCCCC2)Oc2ccccc2)[C@H]1O]

A mixture of intermediate Example 6 (100 mg, 0.167 mmol), isobutyric acid (0.031 mL, 0.333 mmol), and N,N-diisopropylcarbodiimide (0.052 mL, 0.333 mmol) in DMF (2 mL) was stirred at room temperature for 20 min and DMAP (20.34 mg, 0.167 mmol) was added. The resulting mixture was stirred at room temperature for 1 h and purified by preparative HPLC (Phenomenex Gemini-NX 10μ C18 110° A 250×30 mm column, 0%-100% acetonitrile/water gradient in 25 min run) to afford a mixture of regioisomers (39 mg, 35%, 1:3.69 regioisomeric mixture) as a syrup, which was then separated by SFC (30% ethanol, column AD-H 4.6×100 mm).

First eluting regioisomer: ¹H NMR (400 MHz, Methanol-d4) δ 7.83 (s, 1H), 7.31 (dd, J=8.6, 7.2 Hz, 2H), 7.26-7.13 (m, 3H), 6.92 (d, J=4.5 Hz, 1H), 6.76 (d, J=4.6 Hz, 1H), 5.66-5.56 (m, 2H), 4.78 (d, J=5.7 Hz, 1H), 4.63 (dt, J=8.7, 4.6 Hz, 1H), 4.45 (dd, J=11.1, 6.7 Hz, 1H), 4.37 (dd, J=11.1, 5.7 Hz, 1H), 3.85 (dq, J=9.9, 7.1 Hz, 1H), 2.68 (p, J=7.0 Hz, 1H), 1.79-1.59 (m, 4H), 1.56-1.42 (m, 1H), 1.42-1.17 (m, 14H). ³¹P NMR (162 MHz, Methanol-d4) δ 3.25. LCMS: MS m/z=671.18 [M+1]; t_{R}=1.05 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μl/min. HPLC: t_{R}=5.48 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 206. (2R,3S,4S,5S)-5-(4-aminopyrrolo[2,1-f][1,2,4]triazin-7-yl)-2-cyano-2-((((S)—(((S)-1-(cyclohexyloxy)-1-oxopropan-2-yl)amino)(phenoxy)phosphoryl)oxy)methyl)-4-hydroxytetrahydrofuran-3-yl isobutyrate

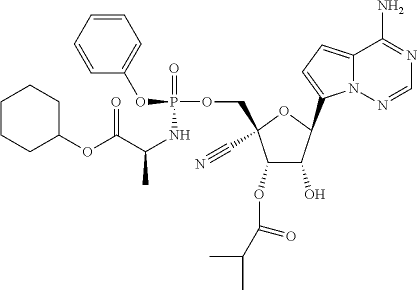 [SMILES: CC(C)C(=O)O[C@H]1[C@@H](O)[C@H](c2ccc3c(N)ncnn23)O[C@]1(C#N)CO[P@@](=O)(N[C@@H](C)C(=O)OC1CCCCC1)Oc1ccccc1]

Second eluting regioisomer of Example 205: ¹H NMR (400 MHz, Methanol-d4) δ 7.84 (s, 1H), 7.33 (t, J=7.9 Hz, 2H), 7.27-7.14 (m, 3H), 6.89 (d, J=4.6 Hz, 1H), 6.77 (d, J=4.6 Hz, 1H), 5.52 (d, J=5.7 Hz, 1H), 5.48 (d, J=6.9 Hz, 1H), 4.91-4.85 (m, 1H), 4.68 (dq, J=8.8, 4.2 Hz, 1H), 4.40 (qd, J=10.9, 5.9 Hz, 2H), 3.94-3.82 (m, 1H), 2.76 (p, J=7.0 Hz, 1H), 1.72 (dd, J=31.4, 10.5 Hz, 4H), 1.56-1.46 (m, 1H), 1.46-1.17 (m, 14H). ³¹P NMR (162 MHz, Methanol-d4) δ 3.16. LCMS: MS m/z=671.18 [M+1]; t_{R}=1.05 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μl/min. HPLC: t_{R}=5.61 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 207. 2-ethylbutyl ((R)-(((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

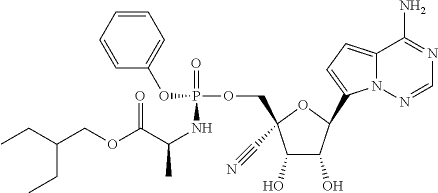 [SMILES: CCC(CC)COC(=O)[C@H](C)N[P@@](=O)(OC[C@@]1(C#N)O[C@@H](c2ccc3c(N)ncnn23)[C@H](O)[C@@H]1O)Oc1ccccc1]

Resolution of the Sp and Rp diastereomers of Example 34 from WO 2015/069939. The product was purified via chiral preparatory SFC (Chiralpak AD-H, 30% Ethanol isocratic) to afford the diastereomers:

First Eluting Diastereomer of Example 34 from WO 2015/069939: ¹H NMR (400 MHz, methanol-d₄) δ 7.78 (s, 1H), 7.32-7.24 (m, 2H), 7.19-7.10 (m, 3H), 6.84 (d, J=4.5 Hz, 1H), 6.72 (d, J=4.5 Hz, 1H), 5.51 (d, J=5.0 Hz, 1H), 4.63 (t, J=5.3 Hz, 1H), 4.54-4.43 (m, 2H), 4.36 (m, 1H), 4.07-3.84 (m, 3H), 1.53-1.42 (m, 1H), 1.38-1.24 (m, 7H), 0.86 (t, J=7.5 Hz, 6H). ³¹P NMR (162 MHz, methanol-d₄) δ 3.26 (s). HPLC: t_{R}=5.068 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

Second Eluting Diastereomer: Example 25. HPLC: t_{R}=5.080 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 208

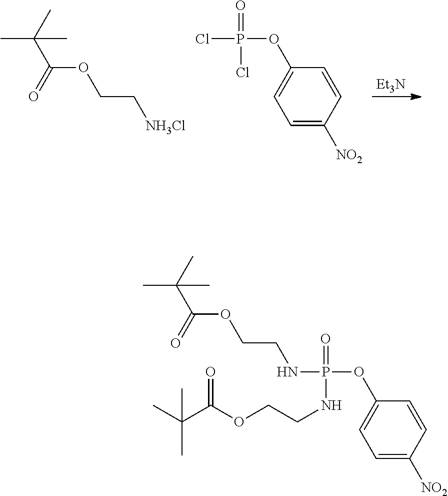

(((4-Nitrophenoxy)phosphoryl)bis(azanediyl))bis(ethane-2,1-diyl) bis(2,2-dimethylpropanoate). Triethylamine (2.28 mL, 16.4 mmol) was added to a solution of 2-(chloro-λ5-azaneyl)ethyl pivalate (1.5 g, 7.8 mmol) and 4-nitrophenyl phosphorodichloridate (1.0 g, 3.9 mmol) in dichloromethane (23 mL) at 0° C. under an argon atmosphere. After 3.5 h, the reaction mixture was diluted with dichloromethane (50 mL), washed with saturated an aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the intermediate. LCMS: MS m/z=474.09 [M+1], t_{R}=1.31 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.799 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min.

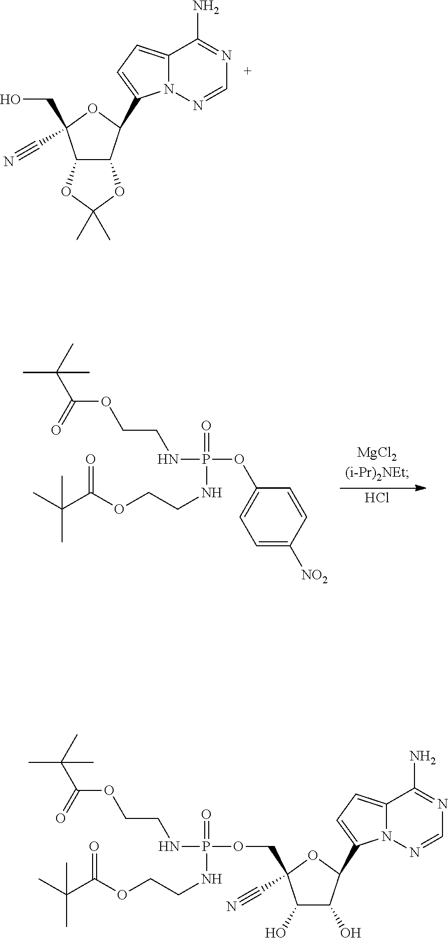 [SMILES: CC1(C)O[C@H]2[C@H](c3ccc4c(N)ncnn34)O[C@](C#N)(CO)[C@H]2O1 ; CC(C)(C)C(=O)OCCNP(=O)(NCCOC(=O)C(C)(C)C)Oc1ccc([N+](=O)[O-])cc1 ; Cl ; CC(C)(C)C(=O)OCCNP(=O)(NCCOC(=O)C(C)(C)C)OC[C@@]1(C#N)O[C@@H](c2ccc3c(N)ncnn23)[C@H](O)[C@@H]1O]

Acetonitrile (1 mL) was added to a mixture of Intermediate 4 (70.0 mg, 0.211 mmol), (((4-nitrophenoxy)phosphoryl)bis(azanediyl))bis(ethane-2,1-diyl) bis(2,2-dimethylpropanoate) (100 mg, 0.211 mmol), and magnesium chloride (20.0 mg, 0.211 mmol) at RT. The mixture was heated to 50° C. for 5 min, and N,N-diisopropylethylamine (0.092 mL, 0.53 mmol) was added. After 2 h, the reaction mixture was allowed to cool to RT, and concentrated aqueous hydrochloric acid solution (0.25 mL) was added dropwise. After 30 min, the reaction mixture was diluted with ethyl acetate (20 mL) and the resulting mixture was washed with saturated aqueous sodium carbonate solution (20 mL) and brine (20 mL). The organic layer was dried over anhydrous sodium sulfate and concentrated under reduced pressure. The crude residue was purified via preparatory HPLC (Phenominex Synergi 4 u Hydro-RR 80 Å 150×30 mm column, 0-100% acetonitrile/water gradient, 0.01% TFA) to afford example afford the product as a TFA salt. ¹H NMR (400 MHz, methanol-d₄) δ 8.04 (s, 1H), 7.35 (d, J=4.7 Hz, 1H), 7.00 (d, J=4.7 Hz, 1H), 5.54 (d, J=5.3 Hz, 1H), 4.54 (t, J=5.4 Hz, 1H), 4.39 (d, J=5.5 Hz, 1H), 4.28-4.16 (m, 2H), 4.10-4.04 (m, 2H), 4.03-3.94 (m, 2H), 3.19-3.04 (m, 4H), 1.20 (s, 9H), 1.16 (s, 9H). ³¹P NMR (162 MHz, methanol-d₄) δ 17.21 (s). LCMS: MS m/z=626.14 [M+1], t_{R}=1.12 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.73 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-5.0 min 2-98% ACN, 5.0 min-6.0 min 98% ACN at 2 mL/min. HPLC: t_{R}=4.51 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 209. ((2R,3S,4R,5S)-5-(4-aminopyrrolo[2,1-f][1,2,4]triazin-7-yl)-2-cyano-3,4-dihydroxytetrahydrofuran-2-yl)methyl bis-2-ethylbenzoate phosphate

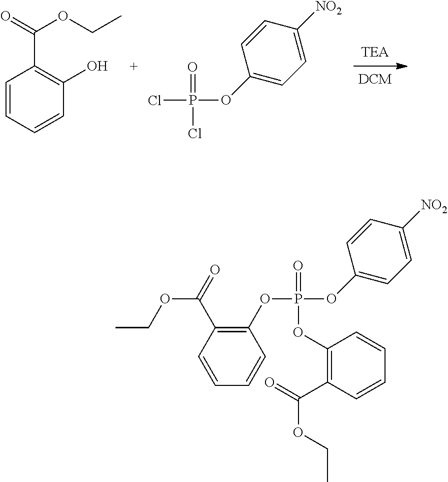 [SMILES: CCOC(=O)c1ccccc1O ; O=[N+]([O-])c1ccc(OP(=O)(Cl)Cl)cc1 ; CCOC(=O)c1ccccc1OP(=O)(Oc1ccc([N+](=O)[O-])cc1)Oc1ccccc1C(=O)OCC]

Bis(ethyl-2-benzoate)-4-nitrophenylphosphate. To a solution of ethyl 2-hydroxybenzoate (3.9 g, 23 mmol) in DCM (10 mL) was added 4-nitrophenyl phosphorodichloridate (3 g, 11.7 mmol) in one portion. The resulting mixture was cooled to 0° C. and triethylamine (3 g, 29 mmol) was added dropwise. The resulting mixture was stirred for 30 min after removal of ice bath and was stirred for overnight. The reaction mixture was then diluted with EtOAc, washed with water and brine, the organic solvent was concentrated in vacuum, and the resulting residue was purified by silica gel column chromatography eluting with 0-100% ethyl acetate in hexanes to afford the intermediate. LCMS: MS m/z=515.41 [M+1], t_{R}=1.42 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=4.25 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

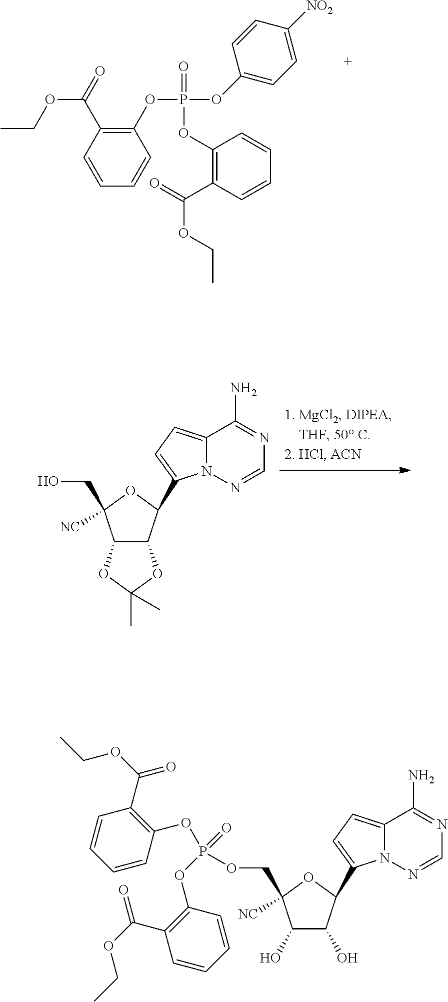 [SMILES: CCOC(=O)c1ccccc1OP(=O)(Oc1ccc([N+](=O)[O-])cc1)Oc1ccccc1C(=O)OCC ; CC1(C)O[C@H]2[C@H](c3ccc4c(N)ncnn34)O[C@](C#N)(CO)[C@H]2O1 ; CCOC(=O)c1ccccc1OP(=O)(OC[C@@]1(C#N)O[C@@H](c2ccc3c(N)ncnn23)[C@H](O)[C@@H]1O)Oc1ccccc1C(=O)OCC]

((2R,3S,4R,5S)-5-(4-aminopyrrolo[2,1-f][1,2,4]triazin-7-yl)-2-cyano-3,4-dihydroxytetrahydrofuran-2-yl)methyl bis-2-ethylbenzoate phosphate. To a mixture of bisethyl-2-benzoate-4-nitrophenylphosphate bis(ethyl-2-benzoate)-4-nitrophenylphosphate (467 mg, 0.9 mmol), Intermediate 4 (200 mg, 0.6 mmol), and MgCl₂ (92 mg, 0.97 mmol) in THF (10 mL) was added N,N-diisopropylethylamine (195 mg, 2 mmol) dropwise. The resulting mixture was stirred at 50° C. for 2 h, reaction mixture was cooled, diluted with EtOAc, washed with water and brine, the organic solvent was evaporated under vacuum, the residue was then dissolved in acetonitrile (8 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was dissolved in DCM and purified by silica gel column chromatography eluting with 0-100% MeOH in DCM to afford the product. ¹H NMR (400 MHz, Acetonitrile-d3) δ 7.91-7.77 (m, 3H), 7.51-7.35 (m, 4H), 7.33-7.22 (m, 2H), 6.70 (q, J=4.5 Hz, 2H), 6.40 (s, 1H), 5.48 (d, J=5.0 Hz, 1H), 4.74-4.56 (m, 3H), 4.33-4.18 (m, 4H), 1.32-1.20 (m, 6H). ³¹P NMR (162 MHz, Acetonitrile-d3) δ 2.67, 2.60. LCMS: MS m/z=668.08 [M+1], t_{R}=1.19 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.88 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 210. ((1r,4S)-4-aminocyclohexyl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

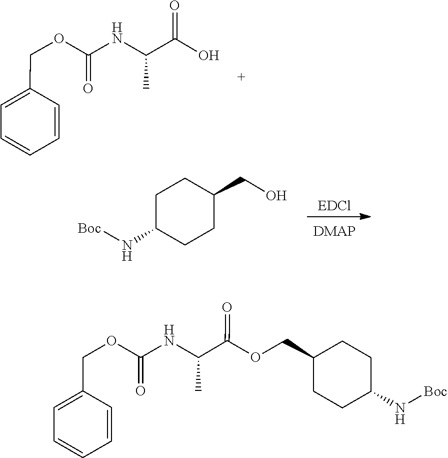 [SMILES: C[C@H](NC(=O)OCc1ccccc1)C(=O)O ; CC(C)(C)OC(=O)N[C@H]1CC[C@H](CO)CC1 ; C[C@H](NC(=O)OCc1ccccc1)C(=O)OC[C@H]1CC[C@H](NC(=O)OC(C)(C)C)CC1]

((1r,4S)-4-((tert-butoxycarbonyl)amino)cyclohexyl)methyl ((benzyloxy) carbonyl)-L-alaninate. Cbz-L-Alanine (223 mg, 1.00 mmol) was dissolved in anhydrous MeCN (10 mL). trans-1-(Boc-amino)-4-(hydroxymethyl)cyclohexane (229 mg, 1.00 mmol) and EDCI (230 mg, 1.2 mmol) were added to the reaction, which was then stirred for 25 min. DMAP (122 mg, 1 mmol) was added in one portion, and the reaction was stirred for 4 h. The reaction mixture was diluted with ethyl acetate (15 mL) and washed with 5% aqueous citric acid solution (2×5 mL), followed with brine (10 mL). Organic extract was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions containing the desired product were combined and concentrated under reduced pressure to give the intermediate. ¹H NMR (400 MHz, chloroform-d) δ 7.41-7.27 (m, 5H), 5.29 (d, J=7.6 Hz, 1H), 5.11 (s, 2H), 4.47-4.24 (m, 2H), 3.96 (d, J=6.6 Hz, 2H), 3.37 (bs, 1H), 2.03 (m, 2H), 1.78 (m, 2H), 1.58 (m, 2H), 1.44 (m, 12H), 1.10 (m, 4H).

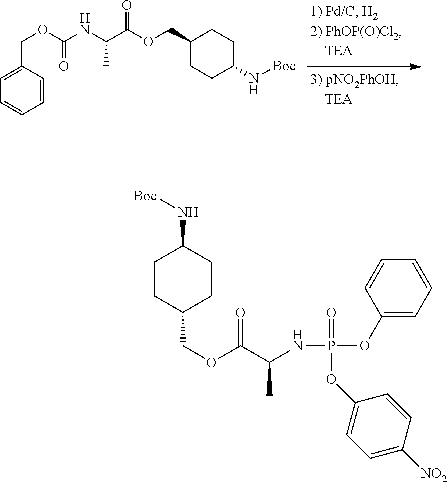 [SMILES: C[C@H](NC(=O)OCc1ccccc1)C(=O)OC[C@H]1CC[C@H](NC(=O)OC(C)(C)C)CC1 ; C[C@H](NP(=O)(Oc1ccccc1)Oc1ccc([N+](=O)[O-])cc1)C(=O)OC[C@H]1CC[C@H](NC(=O)OC(C)(C)C)CC1]

((1r,4S)-4-((tert-butoxycarbonyl)amino)cyclohexyl)methyl ((4-nitrophenoxy)(phenoxy)phosphoryl)-L-alaninate. ((1r,4S)-4-((tert-butoxycarbonyl)amino)cyclohexyl)methyl ((benzyloxy) carbonyl)-L-alaninate (348 mg, 0.800 mmol) was dissolved in 12 mL of anhydrous tetrahydrofuran. Degussa type 10% Palladium on carbon (25 mg) was added to the reaction and then stirred under atmospheric hydrogen for 3 h. Palladium on carbon was filtered off, and the filtrate was used in the next reaction without further purification. Phenyl dichlorophosphate (119 μL, 0.800 mmol) was dissolved in 15 mL anhydrous dichloromethane and stirred under atmospheric nitrogen in an ice bath. The filtrate from above was then added to the reaction solution dropwise and then stirred for 30 min. Triethylamine (120 μL, 0.88 mmol) was added dropwise and stirred for 1 h. p-Nitrophenol (100 mg, 0.72 mmol) was added in one portion. Triethylamine (123 μL, 0.88 mol) was added dropwise, and the reaction mixture was stirred for 2 h at RT. The reaction mixture was then diluted with dichloromethane (10 mL) and washed with water (3×10 mL). The organic extract was dried over anhydrous sodium sulfate and concentrated under reduced pressure. The crude residue was purified via SiO₂ column chromatography (12 g SiO₂ Combiflash HP Gold Column, 0-40% ethyl acetate/hexanes). Fractions containing the desired product were combined and concentrated under reduced pressure to give the intermediate. ¹H NMR (400 MHz, chloroform-d) δ 8.27-8.18 (m, 2H), 7.44-7.30 (m, 4H), 7.27-7.17 (m, 3H), 4.35 (s, 1H), 4.22-4.06 (m, 1H), 3.99-3.88 (m, 2H), 3.85 (t, J=10.6 Hz, 1H), 3.36 (s, 1H), 2.03 (m, 2H), 1.75 (m, 2H), 1.57 (m, 2H), 1.48-1.36 (m, 12H), 1.15-0.98 (m, 4H). ³¹P NMR (162 MHz, chloroform-d) δ 3.12, 3.13. LCMS: MS m/z=478.2 [M+1]; 476.4 [M−1], t_{R}=1.50 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=4.20 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

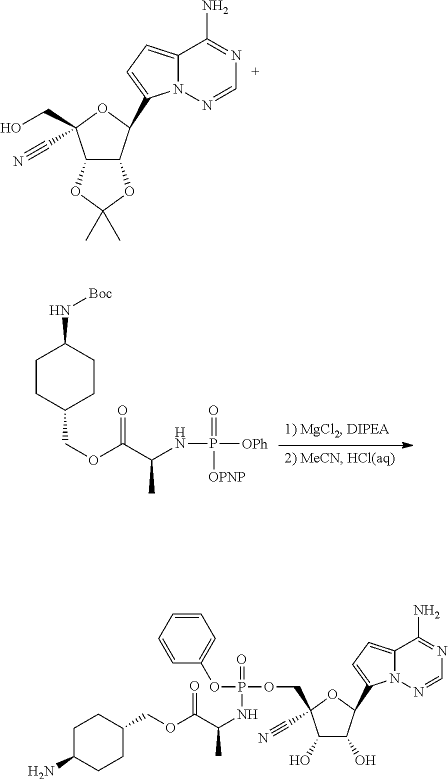

((1r,4S)-4-aminocyclohexyl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Intermediate 4 (83 mg, 0.25 mmol) and ((1r,4S)-4-((tert-butoxycarbonyl)amino)cyclohexyl)methyl ((4-nitrophenoxy)(phenoxy)phosphoryl)-L-alaninate (159 mg, 0.275 mmol) were dissolved in 4 mL of anhydrous tetrahydrofuran. Magnesium chloride (71 mg, 0.75 mmol) was added in one portion. DIPEA (87 μL, 0.5 mmol) was added, and the reaction was stirred at 50° C. for 5 h. The reaction mixture was then diluted with ethyl acetate (10 mL) and washed with water (5×20 mL) and then with brine (10 mL). The organic extract was dried over anhydrous sodium sulfate and concentrated under reduced pressure. The resulting material was dissolved in 5 mL of MeCN and stirred in an ice bath. Concentrated aqueous hydrochloric acid solution (12 N, 300 p L) was added dropwise and the reaction mixture was stirred in an ice bath for 3 h. The reaction mixture was then diluted with ethyl acetate (10 mL) and washed with saturated aqueous sodium bicarbonate solution (10 mL) and followed with brine (5 mL). The aqueous layer was back extracted with a MeOH/ethyl acetate solution (1:1, 5×5 mL). The organic extracts were combined, dried over anhydrous sodium sulfate and concentrated under reduced pressure. The crude product was purified with prep HPLC under neutral conditions (5-100% MeCN/water). Combined fractions and freeze-dried to give the product. ¹H NMR (400 MHz, methanol-d₄) δ 7.79 (m, 1H), 7.39-7.26 (m, 2H), 7.26-7.11 (m, 3H), 6.85 (m, 1H), 6.78-6.70 (m, 1H), 5.50 (m, 1H), 4.68-4.28 (m, 5H), 4.00-3.81 (m, 3H), 2.97 (m, 1H), 2.08-1.94 (m, 2H), 1.90-1.75 (m, 2H), 1.60 (m, 1H), 1.42-1.21 (m, 5H), 1.19-1.00 (m, 2H). ³¹P NMR (162 MHz, methanol-d₄) δ 3.16, 3.29. LCMS: MS m/z=630.2 [M+1]; 628.2 [M−1], t_{R}=0.86 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.02 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=3.357, 3.383 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 211. Cyclohexyl ((S)-(((3aS,4R,6S,6aS)-6-(4-aminopyrrolo[2,1-f][1,2,4]triazin-7-yl)-4-cyano-2,2-dimethyltetrahydrofuro[3,4-d][1,3]dioxol-4-yl)methoxy)(phenoxy)phosphoryl)-L-alaninate

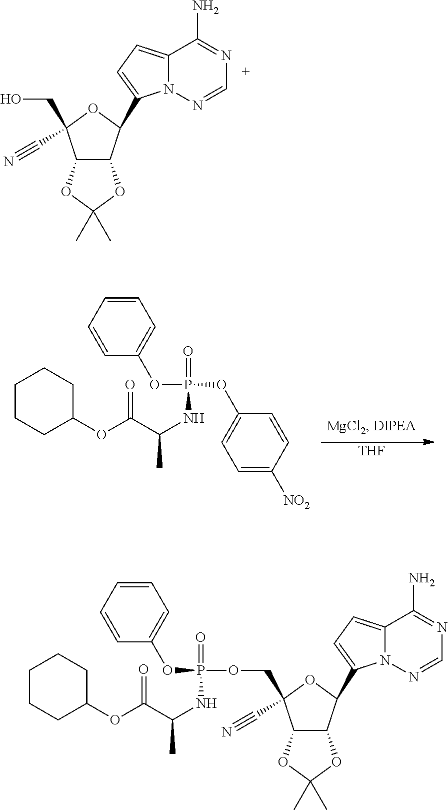 [SMILES: CC1(C)O[C@H]2[C@H](c3ccc4c(N)ncnn34)O[C@](C#N)(CO)[C@H]2O1 ; C[C@H](N[P@](=O)(Oc1ccccc1)Oc1ccc([N+](=O)[O-])cc1)C(=O)OC1CCCCC1 ; C[C@H](N[P@](=O)(OC[C@@]1(C#N)O[C@@H](c2ccc3c(N)ncnn23)[C@@H]2OC(C)(C)O[C@@H]21)Oc1ccccc1)C(=O)OC1CCCCC1 ; "THF"]

To a mixture of Intermediate 4 (50 mg, 0.151 mmol), Intermediate 74 (101.5 mg, 0.226 mmol), and MgCl₂ (22 mg, 0.226 mmol) in THF (2 mL) was added N,N-diisopropylethylamine (0.1 mL, 0.574 mmol) dropwise. The resulting mixture was stirred at 50° C. for 2 h, cooled, and purified by prep HPLC (Phenomenex Gemini-NX 10μ C18 110° A 250×30 mm column, 0%-100% acetonitrile/water gradient in 25 min run) to afford the product. ¹H NMR (400 MHz, Acetonitrile-d3) δ 7.89 (s, 1H), 7.36 (dd, J=8.6, 7.2 Hz, 2H), 7.27-7.16 (m, 3H), 6.77 (q, J=4.5 Hz, 2H), 6.43 (s, 2H), 5.66 (d, J=3.4 Hz, 1H), 5.28 (dd, J=6.6, 3.4 Hz, 1H), 5.10 (d, J=6.6 Hz, 1H), 4.67 (dq, J=8.5, 3.9 Hz, 1H), 4.45 (dd, J=12.3, 10.0 Hz, 1H), 4.39 (dd, J=10.7, 6.6 Hz, 1H), 4.33 (dd, J=10.7, 5.6 Hz, 1H), 3.90 (tq, J=9.7, 7.1 Hz, 1H), 1.81-1.60 (m, 7H), 1.55-1.45 (m, 1H), 1.43-1.19 (m, 11H). ³¹P NMR (162 MHz, Acetonitrile-d3) δ 2.61. LCMS: MS m/z=641.29 [M+1]; t_{R}=1.15 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μl/min.

### Example 212. Cyclohexyl ((S)-(((2R,3S,4R,5S)-2-cyano-3,4-dihydroxy-5-(4-octanamidopyrrolo[2,1-f][1,2,4]triazin-7-yl)tetrahydrofuran-2-yl)methoxy)(phenoxy)phosphoryl)-L-alaninate

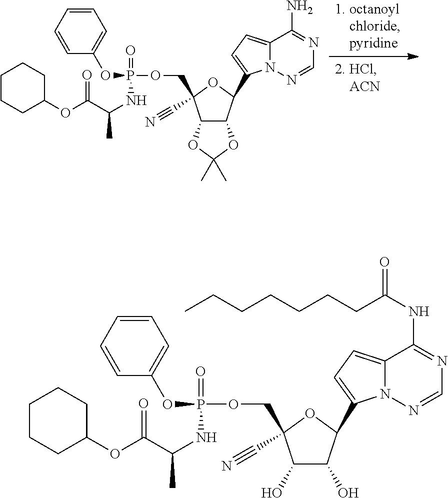 [SMILES: C[C@H](N[P@](=O)(OC[C@@]1(C#N)O[C@@H](c2ccc3c(N)ncnn23)[C@@H]2OC(C)(C)O[C@@H]21)Oc1ccccc1)C(=O)OC1CCCCC1 ; CCCCCCCC(=O)Nc1ncnn2c([C@@H]3O[C@](C#N)(CO[P@@](=O)(N[C@@H](C)C(=O)OC4CCCCC4)Oc4ccccc4)[C@@H](O)[C@H]3O)ccc12]

To a solution of Example 211 (39 mg, 0.061 mmol) in DCM (2 mL) were added pyridine (0.049 mL, 0.609 mmol) and octanoyl chloride (0.016 mL, 0.091 mmol) sequentially. The resulting mixture was stirred for 3 h and quenched by adding methanol (0.1 mL), diluted with EtOAc, washed with water, and dried with sodium sulfate, concentrated in vacuo, and co-evaporated with toluene several time. The obtained residue was dissolved in acetonitrile (2 mL), and c-HCl (0.1 mL) added. The resulting mixture was stirred at room temperature for 1 h and purified by preparative HPLC (Phenomenex Gemini-NX 10μ C18 110° A 250×30 mm column, 0%-100% acetonitrile/water gradient in 25 min run) to afford the product. ¹H NMR (400 MHz, Acetonitrile-d3) δ 8.83 (s, 1H), 8.17 (s, 1H), 7.36 (dd, J=8.6, 7.2 Hz, 2H), 7.23 (dq, J=7.5, 3.5, 2.4 Hz, 3H), 7.16 (d, J=4.7 Hz, 1H), 6.92 (d, J=4.7 Hz, 1H), 5.56 (d, J=4.6 Hz, 1H), 4.66 (dt, J=8.7, 4.4 Hz, 1H), 4.58 (t, J=5.2 Hz, 1H), 4.47 (s, 1H), 4.44-4.23 (m, 4H), 4.03 (s, 1H), 3.90 (ddt, J=16.7, 9.5, 7.1 Hz, 1H), 2.68 (t, J=7.4 Hz, 2H), 1.70 (p, J=7.5 Hz, 2H), 1.49 (d, J=10.0 Hz, 1H), 1.45-1.21 (m, 20H), 0.95-0.86 (m, 3H). ³¹P NMR (162 MHz, Acetonitrile-d3) δ 2.79. LCMS: MS m/z=727.37 [M+1]; t_{R}=1.35 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=6.51 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 213. quinuclidin-4-ylmethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

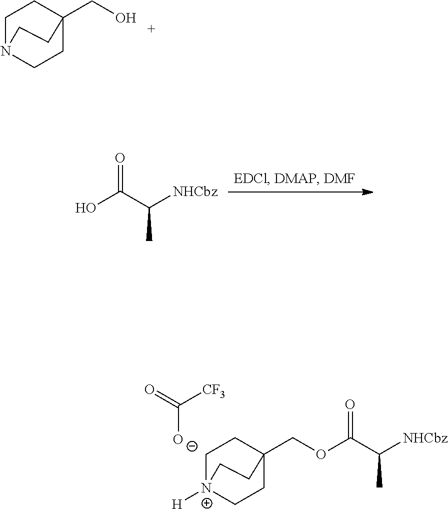 [SMILES: [OH2+]CC12CCN(CC1)CC2 ; C[C@H](NC(=O)OCc1ccccc1)C(=O)O ; O=C([O-])C(F)(F)F ; C[C@H](NC(=O)OCc1ccccc1)C(=O)OCC12CC[NH+](CC1)CC2]

(((((Benzyloxy)carbonyl)-L-alanyl)oxy)methyl)quinuclidin-1-ium 2,2,2-trifluoroacetate. To a mixture of quinuclidin-4-methanol (250 mg, 1.770 mmol), N-Cbz-L-alanine (474 mg, 2.123 mmol) and EDCI (357 mg, 2,300 mmol) in DMF (10 mL) was added DMAP (324 mg, 2.652 mmol) and the reaction mixture was stirred at room temperature for overnight, diluted with water, and purified by preparative HPLC (Phenominex Gemini 10 u C18 110 Å 250×21.2 mm column, 5-95% acetonitrile (0.1% TFA)/water (0.1% TFA) gradient in 30 min run) to afford the intermediate. ¹H NMR (400 MHz, DMSO-d6) δ 9.58 (s, 1H), 7.77 (d, J=7.3 Hz, 1H), 7.41-7.25 (m, 5H), 5.02 (s, 2H), 4.10 (p, J=7.3 Hz, 1H), 3.88 (p, J=12.3, 11.7 Hz, 2H), 3.23 (t, J=8.1 Hz, 6H), 1.70-1.61 (m, 5H), 1.59 (s, 1H), 1.28 (d, J=7.3 Hz, 3H). LCMS: MS m/z=347.28 [M+1-TFA]; t_{R}=0.63 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

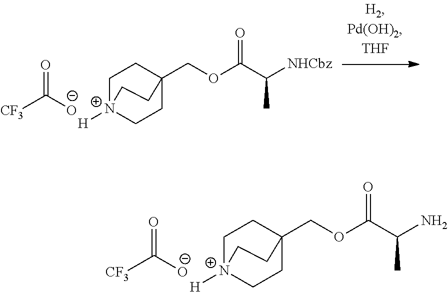 [SMILES: O=C([O-])C(F)(F)F ; C[C@H](NC(=O)OCc1ccccc1)C(=O)OCC12CC[NH+](CC1)CC2 ; O=C([O-])C(F)(F)F ; C[C@H](N)C(=O)OCC12CC[NH+](CC1)CC2]

4-(((L-Alanyl)oxy)methyl)quinuclidin-1-ium 2,2,2-trifluoroacetate. To a solution of (((((Benzyloxy)carbonyl)-L-alanyl)oxy)methyl)quinuclidin-1-ium 2,2,2-trifluoroacetate (545 mg, 1.57 mmol) in THF (10 mL) was added Pd(OH)₂ (700 mg, 0.997 mmol) at room temperature. The resulting mixture was stirred at room temperature for 1 h, filtered, and concentrated in vacuo. The obtained residue was co-evaporated with DCM several times and the resulting intermediate was dried under high vacuum overnight and used in next reaction. LCMS: MS m/z=213.14 [M+1-TFA]; t_{R}=0.13 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

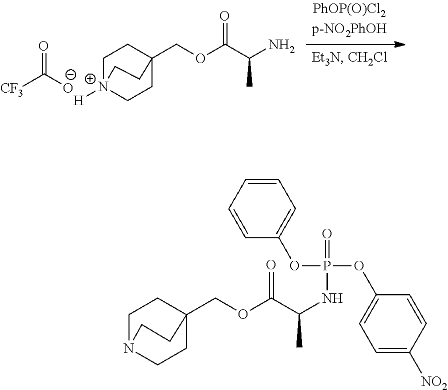 [SMILES: O=C([O-])C(F)(F)F ; C[C@H](N)C(=O)OCC12CC[NH+](CC1)CC2 ; C[C@H](NP(=O)(Oc1ccccc1)Oc1ccc([N+](=O)[O-])cc1)C(=O)OCC12CCN(CC1)CC2]

Quinuclidin-4-ylmethyl((4-nitrophenoxy)(phenoxy) phosphoryl)-L-alaninate. DCM (100 mL) was added to the mixture of 4-(((L-Alanyl)oxy)methyl)quinuclidin-1-ium 2,2,2-trifluoroacetate (399 mg, 1.223 mmol) and triethylamine (0.169 mL, 1.223 mmol) was added to achieve a solution, which was cooled to −78° C. and phenyl dichlorophosphate (0.183 mL, 1.223 mmol) added quickly. Triethylamine (0.169 mL, 1.223 mmol) in DCM (2 mL) was added over 30 min at −78° C. The resulting mixture was stirred for 30 min at the same temperature. Then 4-nitrophenol (170 mg, 1.223 mmol) was added in one portion. Then triethylamine (0.169 mL, 1.223 mmol) in DCM (2 mL) was added over 30 min at −78° C. Then the mixture was stirred for 2 h at room temperature, washed with water and brine, dried over sodium sulfate, and concentrated in vacuo. The residue was purified by silica gel column chromatography (2% TEA containing methanol 0 to 10% in DCM). The isolated fractions having the desired product were combined and concentrated in vacuo to give the crude intermediate, which was used in next reaction without further purification. LCMS: MS m/z=490.40 [M+1]; t_{R}=0.86 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

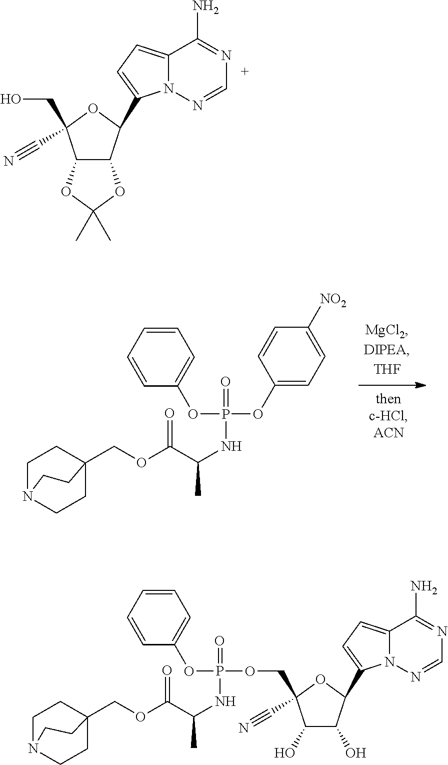 [SMILES: CC1(C)O[C@H]2[C@H](c3ccc4c(N)ncnn34)O[C@](C#N)(CO)[C@H]2O1 ; C[C@H](NP(=O)(Oc1ccccc1)Oc1ccc([N+](=O)[O-])cc1)C(=O)OCC12CCN(CC1)CC2 ; C[C@H](NP(=O)(OC[C@@]1(C#N)O[C@@H](c2ccc3c(N)ncnn23)[C@H](O)[C@@H]1O)Oc1ccccc1)C(=O)OCC12CCN(CC1)CC2]

quinuclidin-4-ylmethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. To a mixture of Intermediate 4 (50 mg, 0.151 mmol), quinuclidin-4-ylmethyl((4-nitrophenoxy)(phenoxy) phosphoryl)-L-alaninate (111 mg, 0.226 mmol, crude), and MgCl₂ (22 mg, 0.226 mmol) in THF (2 mL) was added N,N-diisopropylethylamine (0.1 mL, 0.574 mmol) dropwise. The resulting mixture was stirred at 50° C. for 2 h, cooled, and purified by prep HPLC (Phenomenex Gemini-NX 10μ C18 110° A 250×30 mm column, 0%-100% acetonitrile/water gradient in 25 min run) to give an acetonide intermediate (50 mg, 49%), which was dissolved in acetonitrile (1 mL) and c-HCl (0.05 mL, 0.51 mmol) was added. The resulting mixture was stirred at room temperature for 2 h, neutralized with 5 N NaOH, and purified by preparative HPLC (Phenominex Gemini 10 u C18 110 Å 250×21.2 mm column, 20-65% acetonitrile (0.1% TFA)/water (0.1% TFA) gradient in 30 min run) to afford the product. ¹H NMR (400 MHz, Methanol-d4) δ 7.94 (m, 1H), 7.40-7.12 (m, 6H), 6.88 (m, 1H), 5.52 (m, 1H), 4.56 (m, 1H), 4.49-4.32 (m, 3H), 4.06-3.90 (m, 3H), 3.41-3.29 (m, 6H), 1.81 (m, 6H), 1.33 (d, J=7.1 Hz, 3H). ³¹P NMR (162 MHz, Methanol-d4) δ 3.33, 3.08. LCMS: MS m/z=642.34 [M+1-TFA]; t_{R}=0.64 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=3.28 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 214. ((2R,3S,4R,5S)-5-(4-aminopyrrolo[2,1-f][1,2,4]triazin-7-yl)-2-cyano-3,4-dihydroxytetrahydrofuran-2-yl)methyl bis-cyclopropylmethyl L-alaninate phosphate

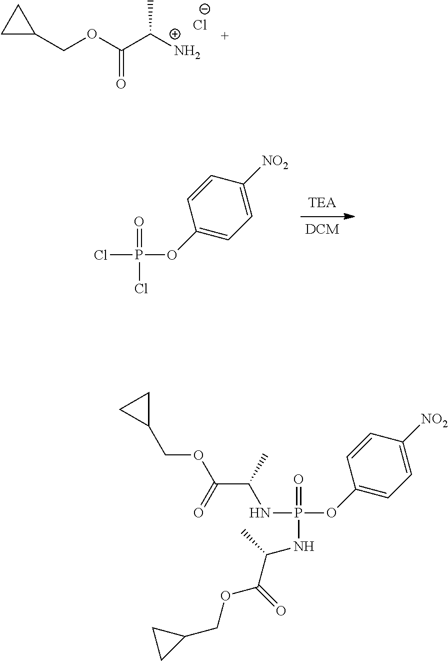

4-nitrophenyl-N,N′-cyclopropylmethyl L-alaninatephosphorodiamidate. To a solution of cyclopropylmethyl L-alaninate HCl salt (146 mg, 0.6 mmol) in DCM (3 mL) was added 4-nitrophenyl phosphorodichloridate (77 mg, 0.3 mmol) in one portion. The resulting mixture was cooled to 0° C. and triethylamine (121 mg, 1.2 mmol) was added dropwise. The resulting mixture was stirred for 30 min after removal of ice bath and was stirred for overnight. The reaction mixture was then diluted with EtOAc, washed with water and brine, the organic solvent was concentrated in vacuum, and the resulting residue was purified by silica gel column chromatography eluting with 0-100% ethyl acetate in hexanes to afford the intermediate. LCMS: MS m/z=470.03 [M+1], t_{R}=1.36 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.01 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

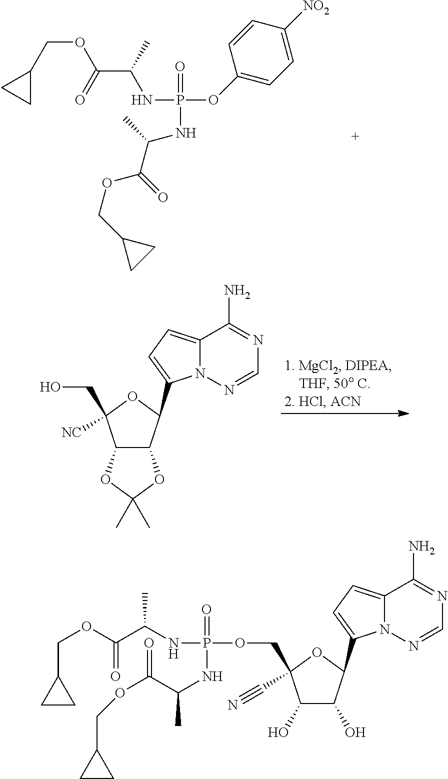 [SMILES: C[C@H](NP(=O)(N[C@@H](C)C(=O)OCC1CC1)Oc1ccc([N+](=O)[O-])cc1)C(=O)OCC1CC1 ; CC1(C)O[C@H]2[C@H](c3ccc4c(N)ncnn34)O[C@](C#N)(CO)[C@H]2O1 ; C[C@H](NP(=O)(N[C@@H](C)C(=O)OCC1CC1)OC[C@@]1(C#N)O[C@@H](c2ccc3c(N)ncnn23)[C@H](O)[C@@H]1O)C(=O)OCC1CC1]

((2R,3S,4R,5S)-5-(4-aminopyrrolo[2,1-f][1,2,4]triazin-7-yl)-2-cyano-3,4-dihydroxytetrahydrofuran-2-yl)methyl bis-cyclopropylmethyl L-alaninate phosphate. To a mixture of 4-nitrophenyl-N,N′-cyclopropylmethyl L-alaninatephosphorodiamidate 4-nitrophenyl-N,N′-cyclopropylmethyl L-alaninatephosphorodiamidate (55 mg, 0.12 mmol), Intermediate 4 (35 mg, 0.11 mmol), and MgCl₂ (15 mg, 0.16 mmol) in THF (2 mL) was added N,N-diisopropylethylamine (34 mg, 0.26 mmol) dropwise. The resulting mixture was stirred at 50° C. for 2 h, reaction mixture was cooled, diluted with EtOAc, washed with water and brine, the organic solvent was evaporated under vacuum, the residue was then dissolved in acetonitrile (2 mL), cooled in ice bath, and con. HCl was added dropwise. The resulting mixture was stirred at room temperature for 2 h, cooled in ice bath, neutralized by dropwise addition of 2 N NaOH and NaHCO₃ solution, diluted with EtOAc (150 mL), washed with water (50 mL) and brine (50 mL). The aqueous phase was extracted with EtOAc (50 mL×2) and the combined organic layer was dried under sodium sulfate, concentrated in vacuum, and residue was purified by Prep HPLC to afford the product. ¹H NMR (400 MHz, Methanol-d4) δ 7.82 (s, 1H), 6.85 (d, J=4.6 Hz, 1H), 6.77 (d, J=4.5 Hz, 1H), 5.50 (d, J=4.9 Hz, 1H), 4.63 (t, J=5.3 Hz, 1H), 4.50 (d, J=5.7 Hz, 1H), 4.31 (dd, J=11.1, 7.0 Hz, 1H), 4.21 (dd, J=11.1, 5.7 Hz, 1H), 4.01-3.73 (m, 6H), 1.37-1.22 (m, 6H), 1.18-0.99 (m, 2H), 0.61-0.44 (m, 4H), 0.32-0.16 (m, 4H). ³¹P NMR (162 MHz, Methanol-d4) δ 13.55. LCMS: MS m/z=622.12 [M+1], t_{R}=1.12 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.58 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 215. Dipentyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-aspartate

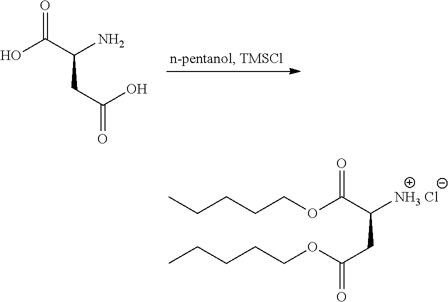

(S)-1,4-Dioxo-1,4-bis(pentyloxy)butan-2-aminium chloride. To a mixture of L-aspartic acid (5 g, 37.56 mmol) and n-pentanol (40 mL, 430.18 mmol) was added TMSCl (12.81 mL, 116.45 mmol) at room temperature. The resulting mixture was stirred at 80° C. for 15 h, cooled to room temperature, concentrated in vacuo at 60° C. Proton NMR indicated small amount of monoester was present. The residue was suspended in EtOAc (400 mL) and sat. NaHCO₃ (100 mL) added. The mixture was stirred for 5 min and the phases were separated. The aqueous layer was extracted with EtOAc. The combined organic layer was concentrated in vacuo and dissolved in DCM (200 mL) and 4 N HCl in dioxane (10 mL) was added under ice bath. The mixture was concentrated in vacuo, triturated in hexanes (100 mL), and the solid obtained by filtration was dried under high vacuum for 15 h to afford the intermediate. ¹H NMR (400 MHz, Chloroform-d) δ 8.86 (d, J=5.0 Hz, 3H), 4.53 (q, J=5.3 Hz, 1H), 4.28-4.14 (m, 2H), 4.11 (td, J=6.9, 1.4 Hz, 2H), 3.32 (dd, J=17.9, 5.0 Hz, 1H), 3.19 (dd, J=17.9, 5.1 Hz, 1H), 1.63 (h, J=7.2 Hz, 4H), 1.30 (dtt, J=5.1, 3.4, 1.9 Hz, 8H), 0.89 (td, J=7.0, 1.5 Hz, 6H). LCMS: MS m/z=274.11 [M+1-HCl]; t_{R}=0.81 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

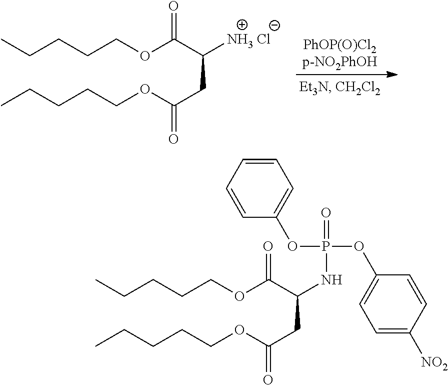

Dipentyl ((4-nitrophenoxy)(phenoxy)phosphoryl)-L-aspartate. (S)-1,4-Dioxo-1,4-bis(pentyloxy)butan-2-aminium chloride (1.0 g, 3.288 mmol) was dissolved in DCM (20 mL) and cooled to −78° C. Phenyl dichlorophosphate (0.48 mL, 3.228 mmol) was added in one portion. Then triethylamine (1.1 mL, 7.22 mmol) in DCM (2 mL) was added over 30 min and stirred until internal temperature reached to 0° C. p-Nitrophenol (449 mg, 3.228 mmol) was added in one portion and triethylamine (0.508 mL, 3.228 mmol) in DCM (2 mL) was added at −78° C. for 30 min. Upon complete addition of triethylamine, the mixture was stirred at room temperature for 15 h, diluted with DCM, washed with sat. NaHCO₃ solution and water, dried with sodium sulfate, and concentrated in vacuo. The obtained residue was purified by silica gel column chromatography (EtOAc 0 to 60% in hexanes) to give the intermediate. ¹H NMR (400 MHz, Chloroform-d) δ 8.22 (m, 2H), 7.48-7.29 (m, 4H), 7.25-7.14 (m, 3H), 4.42-4.26 (m, 2H), 4.11 (m, 2H), 4.02 (m, 2H), 2.97 (m, 1H), 2.78-2.63 (m, 1H), 1.57 (m, 4H), 1.29 (m, 8H), 0.98-0.79 (m, 6H). ³¹P NMR (162 MHz, Chloroform-d) δ −2.60, −2.88. LCMS: MS m/z=551.21 [M+1]; t_{R}=1.44 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

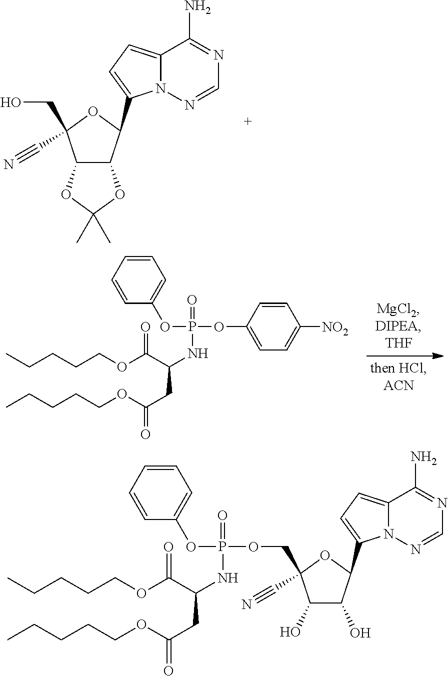

Dipentyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-aspartate. To a mixture of Intermediate 4 (50 mg, 0.151 mmol), dipentyl ((4-nitrophenoxy)(phenoxy)phosphoryl)-L-aspartate (195 mg, 0.354 mmol), and MgCl₂ (22 mg, 0.226 mmol) in THF (2 mL) was added N,N-diisopropylethylamine (0.1 mL, 0.574 mmol) dropwise. The resulting mixture was stirred at 50° C. for 2 h, cooled, diluted with DCM, washed with water and brine, and the aqueous layer extracted with DCM. The combined organic layer was dried with sodium sulfate, and concentrated in vacuo. The obtained residue was dissolved in acetonitrile (2 mL), and treated with c-HCl (0.1 mL), stirred at room temperature for 1 h, and aq.NaHCO₃ (2 mL) added under ice bath. The mixture was purified by preparative HPLC (Phenomenex Gemini-NX 10μ C18 110° A 250×30 mm column, 0%-76% acetonitrile/water gradient in 25 min run) to afford the product. ¹H NMR (400 MHz, Methanol-d4) δ 7.79 (m, 1H), 7.38-7.06 (m, 5H), 6.90-6.77 (m, 1H), 6.73 (m, 1H), 5.51 (m, 1H), 4.62 (m, 1H), 4.52 (m, 1H), 4.48-4.32 (m, 2H), 4.24 (m, 1H), 4.14-3.88 (m, 4H), 2.84-2.57 (m, 2H), 1.64-1.46 (m, 4H), 1.38-1.08 (m, 8H), 0.87 (m, 6H). ³¹P NMR (162 MHz, Methanol-d4) δ 3.22, 3.13. LCMS: MS m/z=703.28 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.76 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min

Resolution of the Sp and Rp diastereomers. The diastereomers were separated by SFC (AD-H, 5 u, 21×250 mm, 30% Ethanol, 55 mL/min) to afford the first eluting isomer and the second eluting isomer:

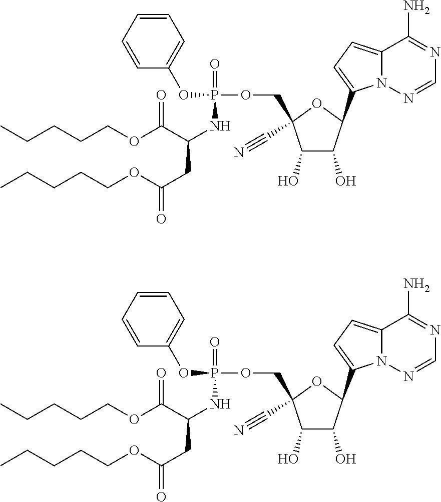 [SMILES: CCCCCOC(=O)C[C@H](N[P@@](=O)(OC[C@@]1(C#N)O[C@@H](c2ccc3c(N)ncnn23)[C@H](O)[C@@H]1O)Oc1ccccc1)C(=O)OCCCCC ; CCCCCOC(=O)C[C@H](N[P@](=O)(OC[C@@]1(C#N)O[C@@H](c2ccc3c(N)ncnn23)[C@H](O)[C@@H]1O)Oc1ccccc1)C(=O)OCCCCC]

### Example 216. First Eluting Diastereomer

¹H NMR (400 MHz, Methanol-d4) δ 7.78 (s, 1H), 7.28 (dd, J=8.7, 7.1 Hz, 2H), 7.21-7.11 (m, 3H), 6.84 (d, J=4.5 Hz, 1H), 6.72 (d, J=4.5 Hz, 1H), 5.51 (d, J=5.1 Hz, 1H), 4.62 (t, J=5.3 Hz, 1H), 4.58-4.48 (m, 2H), 4.38 (dd, J=10.9, 5.5 Hz, 1H), 4.23 (dd, J=11.5, 5.8 Hz, 1H), 4.15-4.02 (m, 2H), 3.99 (td, J=6.7, 2.3 Hz, 2H), 2.70 (d, J=5.9 Hz, 2H), 1.58 (dp, J=9.3, 6.9 Hz, 4H), 1.29 (dq, J=7.2, 3.9, 3.3 Hz, 8H), 0.88 (dt, J=8.5, 6.9 Hz, 6H). ³¹P NMR (162 MHz, Methanol-d4) δ 3.18. LCMS: MS m/z=703.28 [M+1]; t_{R}=1.23 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.77 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 217. Second Eluting Diastereomer

¹H NMR (400 MHz, Methanol-d4) δ 7.80 (s, 1H), 7.32 (dd, J=8.6, 7.2 Hz, 2H), 7.26-7.14 (m, 3H), 6.83 (d, J=4.5 Hz, 1H), 6.72 (d, J=4.5 Hz, 1H), 5.49 (d, J=5.2 Hz, 1H), 4.61 (t, J=5.4 Hz, 1H), 4.45 (d, J=5.6 Hz, 1H), 4.38 (qd, J=10.9, 5.8 Hz, 2H), 4.25 (dt, J=12.0, 6.1 Hz, 1H), 4.04-3.94 (m, 4H), 2.72 (qd, J=16.4, 6.1 Hz, 2H), 1.54 (pd, J=6.8, 3.4 Hz, 4H), 1.26 (pd, J=7.7, 6.4, 2.3 Hz, 8H), 0.86 (td, J=6.8, 4.7 Hz, 6H). ³¹P NMR (162 MHz, Methanol-d4) δ 3.10. LCMS: MS m/z=703.35 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.75 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 218

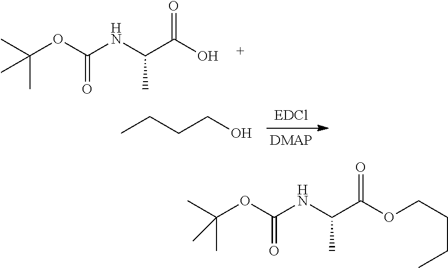

Butyl (tert-butoxycarbonyl)-L-alaninate. Boc-L-alanine (380 mg, 2.0 mmol) was dissolved in anhydrous MeCN (10 mL). 1-Butanol (920 μL, 10.0 mmol) and EDCI (460 mg, 2.4 mmol) were added to the reaction which was then stirred for 15 min. DMAP (240 mg, 2.0 mmol) was added in one portion, and the reaction was stirred for 14 h. The reaction mixture was diluted reaction with ethyl acetate (15 mL) and washed with saturated aqueous sodium bicarbonate solution (2×10 mL), followed with brine (5 mL). The organic extracts were dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions containing the desired product were combined and concentrated under reduced pressure to give the desired product. ¹H NMR (400 MHz, chloroform-d) δ 5.04 (m, 1H), 4.29 (m, 1H), 4.18-4.07 (m, 2H), 1.67-1.59 (m, 2H), 1.44 (s, 9H), 1.38 (m, 5H), 0.93 (t, J=7.4 Hz, 3H).

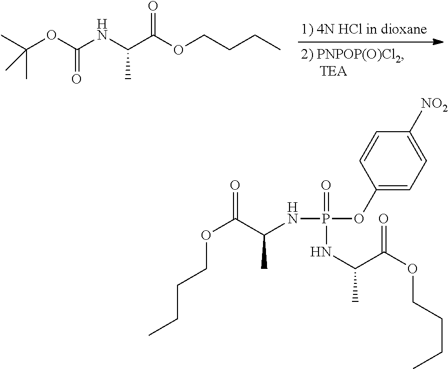

Dibutyl 2,2′-(((4-nitrophenoxy)phosphoryl)bis(azanediyl))(2S,2'S)-dipropionate. Butyl (tert-butoxycarbonyl)-L-alaninate (291 mg, 1.18 mmol) was dissolved in 7 mL of 4M HCl in dioxane and stirred for 1 h. The reaction mixture was concentrated under reduced pressure to give an oil which was then dissolved in anhydrous dichloromethane (10 mL) and stirred under atmospheric nitrogen in an ice bath. 4-Nitrophenyl phosphorodichloridate (152 mg, 0.59 mmol) was added in one portion, and the reaction was stirred for 10 min. Triethylamine (270 μL, 1.95 mmol) was dissolved in 1 mL of anhydrous dichloromethane and added to the reaction solution dropwise. The reaction mixture was stirred for 1 h. Triethylamine (270 μL, 1.95 mmol) was dissolved with 700 μL of anhydrous dichloromethane and added to reaction dropwise. The reaction mixture was stirred for 16 h at RT. The reaction mixture was diluted with dichloromethane (15 mL) and washed with water (3×20 mL). The organic extract was dried over anhydrous sodium sulfate and concentrated under reduced pressure. The crude residue was purified via SiO₂ column chromatography (12 g SiO₂ Combiflash HP Gold Column, 0-50% ethyl acetate/hexanes). Fractions containing the desired product were combined and concentrated under reduced pressure to give the product. ¹H NMR (400 MHz, chloroform-d) δ 8.27-8.15 (m, 2H), 7.43-7.34 (m, 2H), 4.19-3.98 (m, 5H), 3.80-3.61 (m, 1H), 3.58 (m, 2H), 1.67-1.59 (m, 4H), 1.45-1.30 (m, 10H), 0.93 (m, 6H). ³¹P NMR (162 MHz, chloroform-d) δ 7.93. LCMS: MS m/z=474.0 [M+1]; 472.1 [M−1], t_{R}=1.46 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=4.02 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

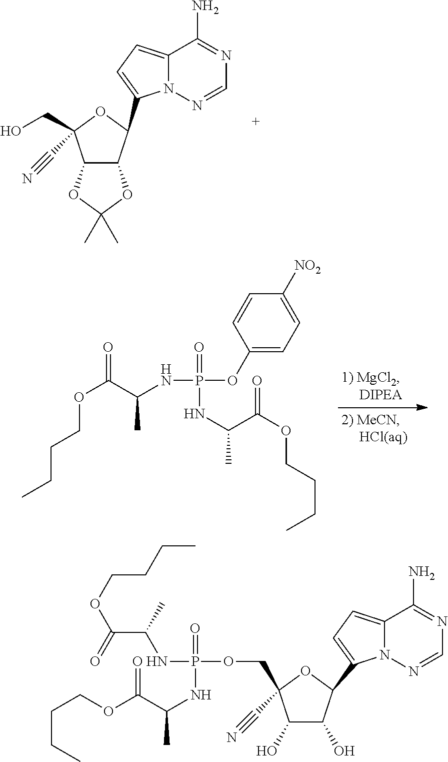 [SMILES: CC1(C)O[C@H]2[C@H](c3ccc4c(N)ncnn34)O[C@](C#N)(CO)[C@H]2O1 ; CCCCOC(=O)[C@H](C)NP(=O)(N[C@@H](C)C(=O)OCCCC)Oc1ccc([N+](=O)[O-])cc1 ; CCCCOC(=O)[C@H](C)NP(=O)(N[C@@H](C)C(=O)OCCCC)OC[C@@]1(C#N)O[C@@H](c2ccc3c(N)ncnn23)[C@H](O)[C@@H]1O]

Intermediate 4 (50 mg, 0.15 mmol) and dibutyl 2,2′-(((4-nitrophenoxy)phosphoryl)bis(azanediyl))(2S,2'S)-dipropionate (71 mg, 0.15 mmol) were combined and dissolved in 1.5 mL of anhydrous tetrahydrofuran. Magnesium chloride (73 mg, 0.45 mmol) was added in one portion. DIPEA (52 μL, 0.3 mmol) was added, and the reaction was stirred at 50° C. for 16 h.

More magnesium chloride (73 mg) and DIPEA (52 μL) were added, and the reaction was stirred at 50° C. for 6 h. The reaction mixture was diluted with ethyl acetate (10 mL) and washed with water (5×10 mL) and then with brine (5 mL). The organic extract was dried over anhydrous sodium sulfate and concentrated under reduced pressure. The residue was dissolved in MeCN (5 mL) and stirred in an ice bath. Concentrated HCl (12 N aq, 300 μL) was added dropwise and then stirred in an ice bath for 1 h. The reaction mixture was diluted with ethyl acetate (10 mL) and was washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-50% ethyl acetate/hexanes). Fractions containing the desired product were combined and concentrated under reduced pressure. The material was dissolved in MeCN and water and freeze-dried to give the product. ¹H NMR (400 MHz, methanol-d₄) δ 7.82 (s, 1H), 6.85 (d, J=4.5 Hz, 1H), 6.77 (d, J=4.5 Hz, 1H), 5.50 (d, J=4.9 Hz, 1H), 4.62 (dd, J=5.7, 4.9 Hz, 1H), 4.50 (d, J=5.7 Hz, 1H), 4.31 (dd, J=11.1, 7.0 Hz, 1H), 4.20 (dd, J=11.1, 5.7 Hz, 1H), 4.16-3.95 (m, 4H), 3.88 (m, 2H), 1.66-1.52 (m, 4H), 1.44-1.29 (m, 7H), 1.26 (m, 3H), 0.92 (m, 6H). ³¹P NMR (162 MHz, methanol-d₄) δ 13.59. LCMS: MS m/z=626.2 [M+1]; 624.3 [M−1], t_{R}=1.20 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=2.85 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=4.752 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 219

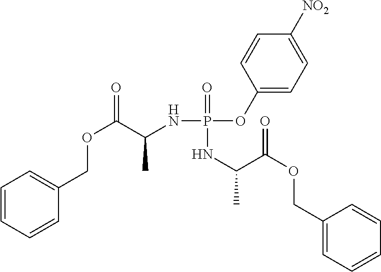 [SMILES: C[C@H](NP(=O)(N[C@@H](C)C(=O)OCc1ccccc1)Oc1ccc([N+](=O)[O-])cc1)C(=O)OCc1ccccc1]

Dibenzyl 2,2′-(((4-nitrophenoxy)phosphoryl)bis(azanediyl))(2S,2'S)-dipropionate. 4-Nitrophenyl phosphorodichloridate (1.28 g, 5 mmol) was mixed with 10 mL of anhydrous dichloromethane and stirred under atmospheric nitrogen in an ice bath. A solution of L-Alanine benzyl ester hydrogen chloride (2.16 g, 10 mmol) in anhydrous dichloromethane (10 mL) was added followed by triethylamine (1.74 mL, 12.5 mmol). The resulting mixture was added to the reaction mixture dropwise. The reaction mixture was stirred for 10 min. Triethylamine (1.5 mL) was dissolved in 10 mL of anhydrous dichloromethane and added to the reaction solution dropwise. The reaction mixture was stirred for 3 h, and was diluted with dichloromethane (15 mL) and washed with water (3×20 mL). The organic extract was dried over anhydrous sodium sulfate and concentrated under reduced pressure. The crude residue was purified via SiO₂ column chromatography (40 g SiO₂ Combiflash HP Gold Column, 0-50% ethyl acetate/hexanes). Fractions containing the desired product were combined and concentrated under reduced pressure to give the product. ¹H NMR (400 MHz, chloroform-d) δ 8.20-8.05 (m, 2H), 7.41-7.19 (m, 12H), 5.14 (s, 4H), 4.18-4.00 (m, 2H), 3.54 (m, 2H), 1.39 (m, 6H). ³¹P NMR (162 MHz, chloroform-d) δ 7.69. LCMS: MS m/z=542.0 [M+1]; 540.0 [M−1], t_{R}=1.44 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=4.00 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

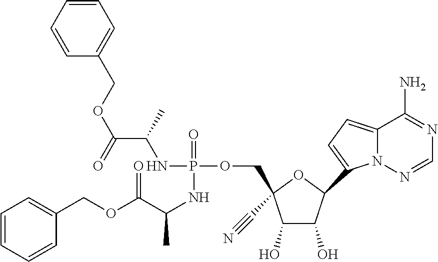 [SMILES: C[C@H](NP(=O)(N[C@@H](C)C(=O)OCc1ccccc1)OC[C@@]1(C#N)O[C@@H](c2ccc3c(N)ncnn23)[C@H](O)[C@@H]1O)C(=O)OCc1ccccc1]

Intermediate 4 (50 mg, 0.15 mmol) and dibenzyl 2,2′-(((4-nitrophenoxy)phosphoryl)bis(azanediyl))(2S,2'S)-dipropionate (97 mg, 0.18 mmol) were mixed and dissolved in 1.5 mL of anhydrous tetrahydrofuran. Magnesium chloride (43 mg, 0.45 mmol) was added in one portion. DIPEA (52 μL, 0.30 mmol) was added and the reaction was stirred at 50° C. for 16 h.

More DIPEA (52 μL) was added and the reaction was stirred at 60° C. for 6 h. The reaction mixture was diluted with ethyl acetate (10 mL) and washed with water (5×10 mL) and then with brine (5 mL). The organic extract was dried over anhydrous sodium sulfate and concentrated under reduced pressure. The resulting material was dissolved in 5 mL of MeCN and stirred in an ice bath. Concentrate HCl(aq) (200 μL) was added dropwise and then stirred in an ice bath for 3 h. Reaction was diluted with ethyl acetate (10 mL) and washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-10% methanol/dichloromethane). Fractions containing the desired product were combined and concentrated under reduced pressure. Material was dissolved in MeCN and water and freeze-dried to give the product. ¹H NMR (400 MHz, methanol-d₄) δ 7.80 (s, 1H), 7.37-7.21 (m, 10H), 6.83 (d, J=4.5 Hz, 1H), 6.74 (d, J=4.5 Hz, 1H), 5.49 (d, J=4.9 Hz, 1H), 5.15-4.96 (m, 4H), 4.61 (dd, J=5.7, 4.9 Hz, 1H), 4.48 (d, J=5.7 Hz, 1H), 4.29 (dd, J=11.1, 7.1 Hz, 1H), 4.17 (dd, J=11.1, 5.7 Hz, 1H), 4.00-3.84 (m, 2H), 1.28 (m, 3H), 1.24-1.20 (m, 3H). ³¹P NMR (162 MHz, methanol-d₄) δ 13.47. LCMS: MS m/z=694.4 [M+1]; 692.2 [M−1], t_{R}=1.22 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.90 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=4.824 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 220

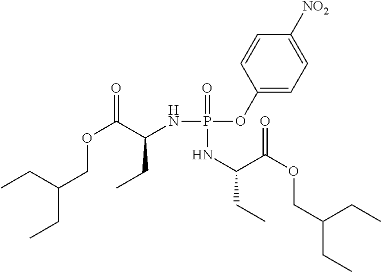 [SMILES: CCC(CC)COC(=O)[C@H](CC)NP(=O)(N[C@@H](CC)C(=O)OCC(CC)CC)Oc1ccc([N+](=O)[O-])cc1]

Bis(2-ethylbutyl) 2,2′-(((4-nitrophenoxy)phosphoryl)bis(azanediyl))(2S,2'S)-dibutyrate. 4-Nitrophenyl phosphorodichloridate (572 mg, 2.23 mmol) was mixed with 10 mL of anhydrous dichloromethane and stirred under atmospheric nitrogen in an ice bath. 2-ethylbutyl (S)-2-aminobutanoate (837 mg, 4.47 mmol) was dissolved in anhydrous dichloromethane (5 mL) and added to reaction dropwise. The reaction was stirred for 10 min. Triethylamine (778 μL, 5.58 mmol) was dissolved with 3 mL of anhydrous dichloromethane and added to the reaction solution dropwise. The reaction mixture was stirred for 3 h. The reaction mixture was diluted with dichloromethane (20 mL) and washed with water (2×20 mL). The organic extract was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). The fractions containing the desired product were combined and concentrated under reduced pressure to afford the product. ¹H NMR (400 MHz, chloroform-d) δ 8.23-8.16 (m, 2H), 7.42-7.34 (m, 2H), 4.09-3.89 (m, 6H), 3.52 (t, J=10.1 Hz, 2H), 1.81 (m, 2H), 1.71 (m, 2H), 1.56-1.45 (m, 2H), 1.40-1.28 (m, 8H), 0.97-0.82 (m, 18H). ³¹P NMR (162 MHz, chloroform-d) δ 8.75. LCMS: MS m/z=558.1 [M+1]; 556.1 [M−1], t_{R}=1.69 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=4.85 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

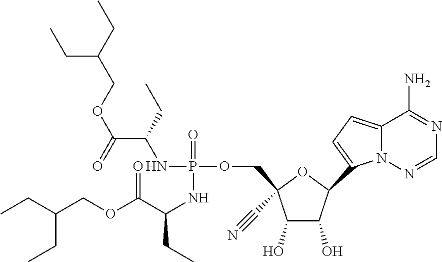 [SMILES: CCC(CC)COC(=O)[C@H](CC)NP(=O)(N[C@@H](CC)C(=O)OCC(CC)CC)OC[C@@]1(C#N)O[C@@H](c2ccc3c(N)ncnn23)[C@H](O)[C@@H]1O]

Intermediate 4 (50 mg, 0.15 mmol) and bis(2-ethylbutyl) 2,2′-(((4-nitrophenoxy)phosphoryl)bis(azanediyl))(2S,2'S)-dibutyrate (109 mg, 0.195 mmol) were mixed and dissolved in 1.5 mL of anhydrous tetrahydrofuran. Magnesium chloride (43 mg, 0.45 mmol) was added in one portion. DIPEA (131 μL, 0.75 mmol) was added and the reaction was stirred at 60° C. for 4 h.

The reaction mixture was diluted with ethyl acetate (10 mL) and washed with aqueous sodium carbonate solution (3×10 mL) and then with brine (5 mL). The organic extract was dried over anhydrous sodium sulfate and concentrated under reduced pressure. The resulting material was dissolved in 5 mL of MeCN and stirred in an ice bath. Concentrated HCl (aq) (200 μL) was added dropwise and the reaction mixture was stirred in an ice bath for 3 h. The reaction mixture was diluted with ethyl acetate (10 mL) and washed with saturated aqueous sodium bicarbonate solution (2×10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-10% methanol/dichloromethane). Fractions containing the desired product were combined and concentrated under reduced pressure which was then dissolved in MeCN and water and freeze-dried to afford the product. ¹H NMR (400 MHz, methanol-d₄) δ 7.82 (s, 1H), 6.85 (d, J=4.5 Hz, 1H), 6.77 (d, J=4.5 Hz, 1H), 5.51 (d, J=4.7 Hz, 1H), 4.62 (dd, J=5.7, 4.7 Hz, 1H), 4.50 (d, J=5.8 Hz, 1H), 4.33 (dd, J=11.1, 7.0 Hz, 1H), 4.22 (dd, J=11.1, 6.1 Hz, 1H), 4.12-3.97 (m, 3H), 3.92 (m, 1H), 3.78 (m, 2H), 3.30 (m, 1H), 1.83-1.41 (m, 6H), 1.41-1.27 (m, 8H), 0.95-0.79 (m, 18H). ³¹P NMR (162 MHz, methanol-d₄) δ 14.07. LCMS: MS m/z=710.3 [M+1]; 708.4 [M−1], t_{R}=1.46 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=3.51 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=5.980 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 221. 2-Ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)-L-alaninate

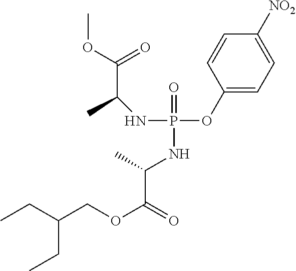 [SMILES: CCC(CC)COC(=O)[C@H](C)NP(=O)(N[C@@H](C)C(=O)OC)Oc1ccc([N+](=O)[O-])cc1]

2-Ethylbutyl ((((S)-1-methoxy-1-oxopropan-2-yl)amino)(4-nitrophenoxy)phosphoryl)-L-alaninate. To a solution of 4-nitrophenyl phosphorodichloridate (1.467 g, 5.732 mmol) in DCM (30 mL) was added methyl L-alaninate (800 mg, 5.732 mmol) in one portion at RT. To the resulting mixture triethylamine (1.6 mL, 11.464 mmol) was added dropwise over 3 min. The resulting mixture was stirred at RT for 30 min and (S)-1-(2-ethylbutoxy)-1-oxopropan-2-aminium chloride (1.202 g, 5.732 mmol) was added in one portion and then triethylamine (1.6 mL, 11.464 mmol) added over 3 min. The resulting mixture was stirred at RT for 30 min, diluted with EtOAc, washed with brine, concentrated in vacuo, and the resulting residue purified by silica gel column chromatography (EtOAc 0 to 60% in hexanes for 45 min and then EtOAc 100% for 15 min) to give the product. ¹H NMR (400 MHz, Chloroform-d) δ 8.21 (m, 2H), 7.41-7.32 (m, 2H), 4.17-3.97 (m, 4H), 3.72 (s, 3H), 3.58 (m, 2H), 1.57-1.46 (m, 1H), 1.41 (m 6H), 1.34 (m, 4H), 0.88 (m, 6H). ³¹P NMR (162 MHz, Chloroform-d) δ 7.88. LCMS m/z=460.06 (M+H), t_{R}=1.19 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

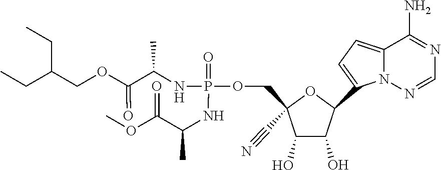 [SMILES: CCC(CC)COC(=O)[C@H](C)NP(=O)(N[C@@H](C)C(=O)OC)OC[C@@]1(C#N)O[C@@H](c2ccc3c(N)ncnn23)[C@H](O)[C@@H]1O]

2-Ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(((S)-1-methoxy-1-oxopropan-2-yl)amino)phosphoryl)-L-alaninate. To a mixture of Intermediate 4 (63 mg, 0.190 mmol), 2-Ethylbutyl ((((S)-1-methoxy-1-oxopropan-2-yl)amino)(4-nitrophenoxy)phosphoryl)-L-alaninate (210 mg, 0.456 mmol), and MgCl₂ (27 mg, 0.285 mmol) in THF (2 mL) was added N,N-diisopropylethylamine (0.126 mL, 0723 mmol) dropwise. The resulting mixture was stirred at 50° C. for 1 h, cooled, concentrated in vacuo, co-evaporated with DCM several times, dissolved acetonitrile (4 mL), and c-HCl (0.4 mL) added. The resulting mixture was stirred at room temperature for 30 min, and NaHCO₃ (3 mL) added under ice bath. The mixture was purified by prep HPLC (Phenomenex Gemini-NX 10μ C18 110° A 250×30 mm column, 0%-76% acetonitrile/water gradient in 25 min run) to afford the product. ¹H NMR (400 MHz, Methanol-d4) δ 7.81 (s, 1H), 6.85 (m, 1H), 6.77 (m, 1H), 5.51 (m, 1H), 4.67-4.60 (m, 1H), 4.51 (m, 1H), 4.31 (m, 1H), 4.21 (m, 1H), 4.11-3.80 (m, 4H), 3.68 (s, 1.27H), 3.63 (s, 1.73H), 1.48 (m, 1H), 1.41-1.21 (m, 10H), 0.87 (m, 6H). ³¹P NMR (162 MHz, Methanol-d4) δ 13.58. LCMS: MS m/z=612.29 [M+1]; t_{R}=0.90 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.49 min (35%), 4.52 min (64%); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

Resolution of the Sp and Rp diastereomers. The product was separated by SFC (IA, 5 u, 21×250 mm, 20% Ethanol) to afford the diastereomers:

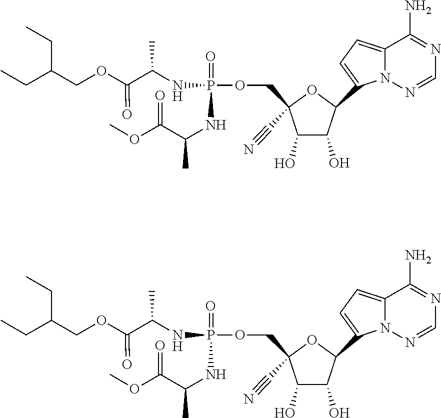 [SMILES: CCC(CC)COC(=O)[C@H](C)N[P@@](=O)(N[C@@H](C)C(=O)OC)OC[C@@]1(C#N)O[C@@H](c2ccc3c(N)ncnn23)[C@H](O)[C@@H]1O ; CCC(CC)COC(=O)[C@H](C)N[P@](=O)(N[C@@H](C)C(=O)OC)OC[C@@]1(C#N)O[C@@H](c2ccc3c(N)ncnn23)[C@H](O)[C@@H]1O]

### Example 222. First Eluting Diastereomer

¹H NMR (400 MHz, Methanol-d4) δ 7.82 (s, 1H), 6.85 (d, J=4.6 Hz, 1H), 6.77 (d, J=4.6 Hz, 1H), 5.50 (d, J=4.9 Hz, 1H), 4.63 (t, J=5.3 Hz, 1H), 4.50 (d, J=5.7 Hz, 1H), 4.30 (dd, J=11.1, 6.9 Hz, 1H), 4.20 (dd, J=11.1, 5.7 Hz, 1H), 4.07 (dd, J=10.9, 5.8 Hz, 1H), 3.98 (dd, J=10.9, 5.7 Hz, 1H), 3.88 (ddq, J=23.9, 9.5, 7.1 Hz, 2H), 3.63 (s, 3H), 1.50 (h, J=6.1 Hz, 1H), 1.42-1.29 (m, 7H), 1.28-1.23 (m, 3H), 0.89 (t, J=7.5 Hz, 6H). ³¹P NMR (162 MHz, Methanol-d4) δ 13.57. LCMS: MS m/z=612.25 [M+1]; t_{R}=0.88 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.46 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 223. Second Eluting Diastereomer

¹H NMR (400 MHz, Methanol-d4) δ 7.82 (s, 1H), 6.85 (d, J=4.5 Hz, 1H), 6.77 (d, J=4.6 Hz, 1H), 5.50 (d, J=4.9 Hz, 1H), 4.68-4.58 (m, 1H), 4.50 (d, J=5.7 Hz, 1H), 4.31 (dd, J=11.1, 7.0 Hz, 1H), 4.20 (dd, J=11.1, 5.7 Hz, 1H), 4.03 (dd, J=10.9, 5.7 Hz, 1H), 3.97-3.81 (m, 3H), 3.68 (s, 3H), 1.55-1.39 (m, J=6.3 Hz, 1H), 1.38-1.28 (m, 7H), 1.26 (d, J=7.2 Hz, 3H), 0.87 (t, J=7.5 Hz, 6H). ³¹P NMR (162 MHz, Methanol-d4) δ 13.58. LCMS: MS m/z=612.29 [M+1]; t_{R}=0.89 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.45 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 224. ethyl 2-(((4aR,6S,7S,7aS)-6-(4-aminopyrrolo[2,1-f][1,2,4]triazin-7-yl)-4a-cyano-7-hydroxy-2-oxidotetrahydro-4H-furo[3,2-d][1,3,2]dioxaphosphinin-2-yl)oxy)benzoate

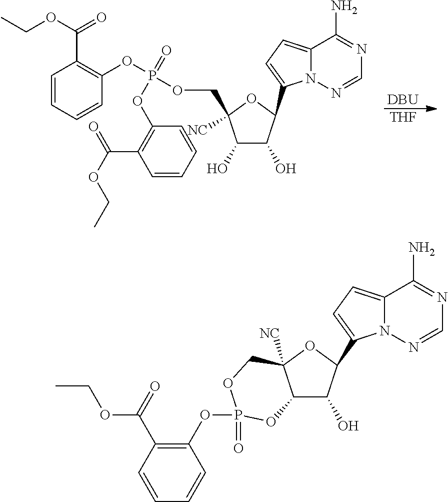

To a solution of Example 209 (10 mg, 0.015 mmol in THF (1 mL) was added DBU (6 mg, 0.037 mmol) dropwise. The resulting mixture was stirred at RT for 2 h, reaction mixture was then diluted with EtOAc, washed with NH₄Cl and brine, the organic solvent was evaporated under vacuum, the residue was then purified by Prep HPLC to afford the product. ¹H NMR (400 MHz, Methanol-d4) δ 8.06-7.93 (m, 1H), 7.78 (s, 1H), 7.69-7.54 (m, 2H), 7.38 (td, J=8.2, 7.7, 1.7 Hz, 1H), 6.85 (q, J=4.6 Hz, 2H), 5.74 (s, 1H), 5.68 (dd, J=5.2, 2.5 Hz, 1H), 5.00-4.89 (m, 1H), 4.78 (d, J=5.2 Hz, 2H), 4.43-4.29 (m, 2H), 1.38 (t, J=7.1 Hz, 3H). ³¹P NMR (162 MHz, Methanol-d4) δ −13.44. LCMS: MS m/z=502.08 [M+1], t_{R}=1.14 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.66 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 225. cyclohexyl ((S)-(((3aS,4R,6S,6aS)-6-(4-aminopyrrolo[2,1-f][1,2,4]triazin-7-yl)-4-cyano-2-ethoxytetrahydrofuro[3,4-d][1,3]dioxol-4-yl)methoxy)(phenoxy)phosphoryl)-L-alaninate

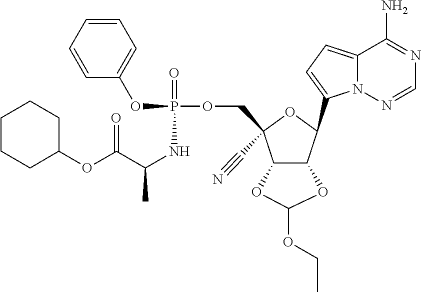

Example 6 (31 mg, 0.052 mmol) was dissolved in DMF (3 mL), p-toluenesulfonic acid (18 mg, 0.1 mmol) and the ortho ester (384 mg, 2.6 mmol) was added under argon at RT. The solution was stirred for 40 min, diluted with AcOEt (50 mL), washed with brine (4×20 mL), sat. aqueous NaHCO₃ (20 mL), and the solvent was evaporated under vacuum. The residue was purified with prep HPLC to afford the product. ¹H NMR (400 MHz, Methanol-d4) δ 7.81 (s, 1H), 7.34 (t, J=7.8 Hz, 2H), 7.27-7.14 (m, 3H), 6.85 (d, J=4.6 Hz, 1H), 6.77 (d, J=4.6 Hz, 1H), 6.12 (s, 1H), 5.83 (d, J=5.2 Hz, 1H), 5.44 (dd, J=7.2, 5.2 Hz, 1H), 5.04 (d, J=7.2 Hz, 1H), 4.70 (dt, J=8.9, 4.7 Hz, 1H), 4.42 (dd, J=10.4, 6.4 Hz, 1H), 4.31 (dd, J=10.4, 5.3 Hz, 1H), 3.97-3.82 (m, 2H), 3.80-3.67 (m, 1H), 1.77 (d, J=6.7 Hz, 2H), 1.69 (d, J=10.0 Hz, 2H), 1.55-1.38 (m, 3H), 1.38-1.25 (m, 9H). ³¹P NMR (162 MHz, Methanol-d4) δ 3.66. LCMS: MS m/z=6.57.11 [M+1], t_{R}=1.41 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.33 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 226. 2-Ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)((2-(2-ethylbutoxy)-2-oxoethyl)amino)phosphoryl)glycinate

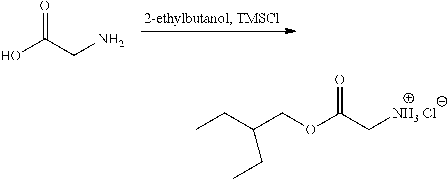

2-(2-Ethylbutoxy)-2-oxoethan-1-aminium chloride. To a mixture of glycine (4.2 g, 55.95 mmol) and 2-ethtylbutanol (60 mL, 489.15 mmol) was added TMSCl (19.08 mL, 173.45 mmol) at room temperature. The resulting mixture was stirred at 70° C. for 15 h, concentrated in vacuo at 60° C., and co-evaporated with toluene several times (33 mL×3). The resulting syrup was suspended in hexanes (70 mL), stirred at room temperature for 15 h, and the precipitated solid collected by filtration. The solid was dried under high vacuum for 2 days at 30° C. to afford the intermediate and used in next reaction. ¹H NMR (400 MHz, Chloroform-d) δ 8.59 (d, J=6.1 Hz, 3H), 4.09 (d, J=5.9 Hz, 2H), 3.99 (q, J=5.8 Hz, 2H), 1.51 (h, J=6.2 Hz, 1H), 1.33 (p, J=7.3 Hz, 4H), 0.86 (t, J=7.4 Hz, 6H). LCMS: MS m/z=159.87 [M+1-HCl]; t_{R}=0.61 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

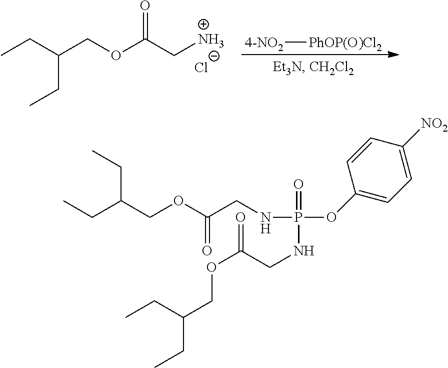

2-Ethylbutyl (((2-(2-ethylbutoxy)-2-oxoethyl)amino)(4-nitrophenoxy)phosphoryl)glycinate. To a solution of 4-nitrophenyl phosphorodichloridate (1.700 g, 6.641 mmol) in DCM (20 mL) was added 2-(2-Ethylbutoxy)-2-oxoethan-1-aminium chloride (2.60 g, 13.28 mmol) in one portion. To the resulting mixture was added trimethylamine (2.76 mL, 19.92 mmol) dropwise over 3 min at room temperature. The resulting mixture was stirred for 30 min at room temperature, diluted with DCM, washed with brine, concentrated in vacuo, and the resulting residue purified by silica gel column chromatography (EtOAc 0 to 60% in hexane) to afford the intermediate. ¹H NMR (400 MHz, Chloroform-d) δ 8.21 (d, J=9.0 Hz, 2H), 7.39 (d, J=8.9 Hz, 2H), 4.08 (d, J=5.9 Hz, 4H), 3.82 (dd, J=11.0, 6.0 Hz, 4H), 3.56 (s, 2H), 1.52 (p, J=6.2 Hz, 2H), 1.41-1.26 (m, 8H), 0.88 (t, J=7.4 Hz, 12H). ³¹P NMR (162 MHz, Chloroform-d) δ 10.05. LCMS: MS m/z=502.16 [M+1]; t_{R}=1.36 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

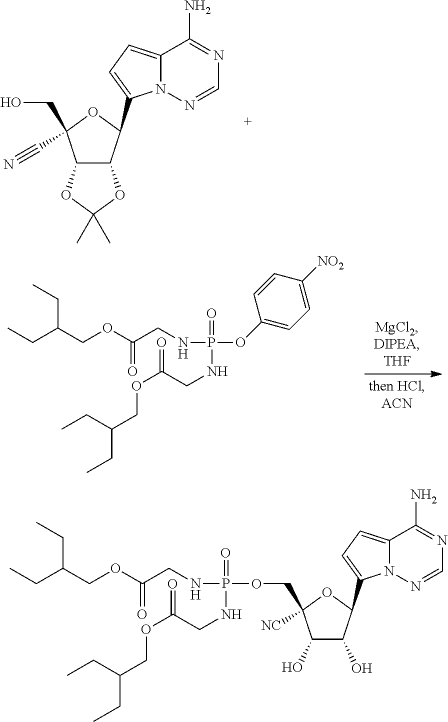 [SMILES: CC1(C)O[C@H]2[C@H](c3ccc4c(N)ncnn34)O[C@](C#N)(CO)[C@H]2O1 ; CCC(CC)COC(=O)CNP(=O)(NCC(=O)OCC(CC)CC)Oc1ccc([N+](=O)[O-])cc1 ; CCC(CC)COC(=O)CNP(=O)(NCC(=O)OCC(CC)CC)OC[C@@]1(C#N)O[C@@H](c2ccc3c(N)ncnn23)[C@H](O)[C@@H]1O]

2-Ethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)((2-(2-ethylbutoxy)-2-oxoethyl)amino)phosphoryl)glycinate. To a mixture of Intermediate 4 (63 mg, 0.190 mmol), 2-Ethylbutyl (((2-(2-ethylbutoxy)-2-oxoethyl)amino)(4-nitrophenoxy)phosphoryl)glycinate (229 mg, 0.456 mmol), and MgCl₂ (27 mg, 0.285 mmol) in THF (2 mL) was added N,N-diisopropylethylamine (0.126 mL, 0723 mmol) dropwise. The resulting mixture was stirred at 50° C. for 2 h, cooled, and purified by preparative HPLC (Phenomenex Gemini-NX 10μ C18 110° A 250×30 mm column, 0%-100% acetonitrile/water gradient in 25 min run). The obtained intermediate was dissolved acetonitrile (2 mL), and c-HCl (0.1 mL) added. The resulting mixture was stirred at room temperature for 30 min, and aq. NaHCO₃ (3 mL) added under ice bath. The mixture was purified by preparative HPLC (Phenomenex Gemini-NX 10μ C18 110° A 250×30 mm column, 0%-76% acetonitrile/water gradient in 25 min run) to afford the product. ¹H NMR (400 MHz, Methanol-d4) δ 7.82 (s, 1H), 6.86 (d, J=4.5 Hz, 1H), 6.77 (d, J=4.5 Hz, 1H), 5.51 (d, J=5.1 Hz, 1H), 4.61 (t, J=5.3 Hz, 1H), 4.46 (d, J=5.6 Hz, 1H), 4.35 (dd, J=11.0, 6.6 Hz, 1H), 4.25 (dd, J=11.0, 6.0 Hz, 1H), 4.05-3.99 (m, 4H), 3.72-3.62 (m, 4H), 1.47 (ddt, J=16.1, 12.2, 6.2 Hz, 2H), 1.39-1.25 (m, 8H), 0.87 (td, J=7.4, 5.8 Hz, 12H). ³¹P NMR (162 MHz, Methanol-d4) δ 16.10. LCMS: MS m/z=654.50 [M+1]; t_{R}=1.10 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.29 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min

### Example 227. Resolution of the Sp and Rp Diastereomers of Example 30

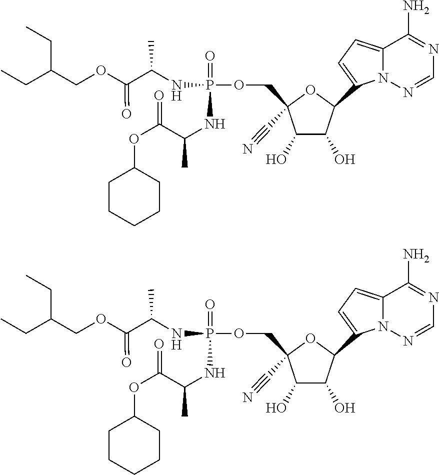 [SMILES: CCC(CC)COC(=O)[C@H](C)N[P@@](=O)(N[C@@H](C)C(=O)OC1CCCCC1)OC[C@@]1(C#N)O[C@@H](c2ccc3c(N)ncnn23)[C@H](O)[C@@H]1O ; CCC(CC)COC(=O)[C@H](C)N[P@](=O)(N[C@@H](C)C(=O)OC1CCCCC1)OC[C@@]1(C#N)O[C@@H](c2ccc3c(N)ncnn23)[C@H](O)[C@@H]1O]

Example 30 (14.34 mg, 0.021 mmol) was separated by Chiralpack (IF, 50 u, 150×4.6 mm, heptane 70%, isopropanol 30%) to afford the diastereomers.

### Example 227. First Eluting Diastereomer

¹H NMR (400 MHz, Acetonitrile-d3) δ 7.90 (s, 1H), 6.82-6.66 (m, 2H), 6.12 (s, 2H), 5.48 (d, J=4.5 Hz, 1H), 4.70 (td, J=8.7, 4.0 Hz, 1H), 4.58 (t, J=5.1 Hz, 1H), 4.49 (d, J=5.7 Hz, 1H), 4.26 (dd, J=11.3, 8.0 Hz, 1H), 4.15 (dd, J=11.4, 7.9 Hz, 1H), 4.08 (dd, J=10.9, 5.7 Hz, 1H), 3.99 (dd, J=10.9, 5.6 Hz, 1H), 3.89 (s, 1H), 3.78 (s, 4H), 1.87-1.75 (m, 2H), 1.75-1.65 (m, 2H), 1.53 (m, 2H), 1.37 (m, 6H), 1.28 (m, 9H), 0.90 (t, J=7.5 Hz, 6H). ³¹P NMR (162 MHz, Acetonitrile-d3) δ 12.54. LCMS: MS m/z=680.40 [M+1]; t_{R}=0.99 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.40 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 228. Second Eluting Diastereomer

¹H NMR (400 MHz, Acetonitrile-d₃) δ 7.90 (s, 1H), 6.83-6.71 (m, 2H), 6.25 (s, 2H), 5.48 (d, J=4.5 Hz, 1H), 4.73 (m, 1H), 4.67 (s, 1H), 4.58 (t, J=5.1 Hz, 1H), 4.49 (d, J=5.7 Hz, 1H), 4.26 (dd, J=11.3, 8.0 Hz, 1H), 4.15 (dd, J=11.3, 7.7 Hz, 1H), 4.08-4.03 (m, 1H), 3.95 (m, 2H), 3.90-3.65 (m, 3H), 1.79 (m, 2H), 1.72 (m, 2H), 1.59-1.22 (m, 17H), 0.88 (t, J=7.5 Hz, 6H). ³¹P NMR (162 MHz, Acetonitrile-d3) δ 12.52. LCMS: MS m/z=680.39 [M+1]; t_{R}=0.97 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.38 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 229. 5-butyl 1-cyclohexyl ((((2R,3S,4R,5S)-5-(4-aminopyrrolo [2,1-f][1,2,4]triazin-7-yl)-2-cyano-3,4-dihydroxytetrahydrofuran-2-yl)methoxy)(phenoxy) phosphoryl)-L-glutamate

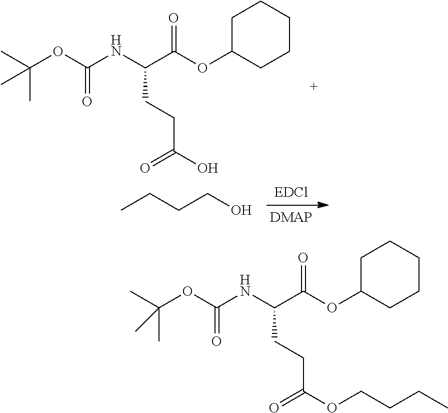

5-butyl 1-cyclohexyl (tert-butoxycarbonyl)-L-glutamate. Boc-L-Glutamic acid alpha cyclohexyl ester (494 mg, 1.5 mmol) was dissolved in anhydrous MeCN (10 mL). 1-Butanol (1.37 mL, 15 mmol) and EDCI (345 mg, 1.8 mmol) were added to the reaction which was then stirred for 45 min. DMAP (183 mg, 1.5 mmol) was added in one portion and the reaction was stirred for 14 h. The reaction mixture was diluted with ethyl acetate (15 mL) and washed with saturated aqueous sodium bicarbonate solution (2×10 mL) followed with brine (5 mL). The organic layer was dried over anhydrous sodium sulfate and concentrated under reduced pressure. The crude residue was purified via SiO₂ column chromatography (12 g SiO₂ Combiflash HP Gold Column, 0-20% ethyl acetate/hexanes). Fractions containing the desired product were combined and concentrated under reduced pressure to give the product. ¹H NMR (400 MHz, chloroform-d) δ 5.10 (m, 1H), 4.86-4.76 (m, 1H), 4.36-4.21 (m, 1H), 4.08 (t, J=6.7 Hz, 2H), 2.48-2.29 (m, 2H), 2.16 (m, 1H), 1.93 (m, 1H), 1.89-1.78 (m, 2H), 1.78-1.66 (m, 2H), 1.66-1.48 (m, 4H), 1.48-1.30 (m, 15H), 0.93 (t, J=7.4 Hz, 3H).

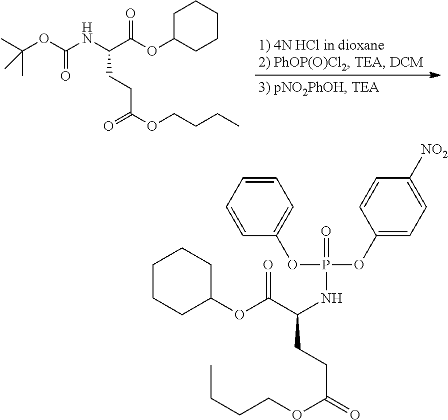 [SMILES: CCCCOC(=O)CC[C@H](NC(=O)OC(C)(C)C)C(=O)OC1CCCCC1 ; CCCCOC(=O)CC[C@H](NP(=O)(Oc1ccccc1)Oc1ccc([N+](=O)[O-])cc1)C(=O)OC1CCCCC1]

5-butyl 1-cyclohexyl ((4-nitrophenoxy)(phenoxy)phosphoryl)-L-glutamate. 5-butyl 1-cyclohexyl (tert-butoxycarbonyl)-L-glutamate (420 mg, 1.09 mmol) was dissolved in 10 mL of 4 M HCl in dioxane and stirred for 2 h. The reaction mixture was concentrated under reduced pressure and the crude residue was used directly in the next step. Phenyl dichlorophosphate (162 μL, 1.09 mmol) was dissolved in 5 mL anhydrous dichloromethane and stirred under atmospheric nitrogen in an ice bath. The crude residue prepared above was dissolved in anhydrous dichloromethane (3 mL) and added to reaction mixture dropwise. The reaction mixture was stirred for 10 min, and triethylamine (334 μL, 2.4 mmol) was dissolved in 3 mL of anhydrous dichloromethane and added to the reaction mixture dropwise. The reaction mixture was stirred for 2 h. p-Nitrophenol (136 mg, 0.981 mmol) was added in one portion. Triethylamine (167 μL, 1.2 mmol) was dissolved in 3 mL anhydrous dichloromethane and added to the reaction dropwise. The reaction mixture was stirred for 16 h, and the reaction mixture was diluted with dichloromethane (15 mL) and washed with water (3×20 mL). The organic layer was dried over anhydrous sodium sulfate and concentrated under reduced pressure. The crude residue was purified via SiO₂ column chromatography (12 g SiO₂ Combiflash HP Gold Column, 0-30% ethyl acetate/hexanes). Fractions containing the desired product were combined and concentrated under reduced pressure to give the intermediate. ¹H NMR (400 MHz, chloroform-d) δ 8.26-8.16 (m, 2H), 7.44-7.29 (m, 4H), 7.25-7.14 (m, 3H), 4.81-4.70 (m, 1H), 4.19-4.08 (m, 1H), 4.03 (m, 2H), 3.96-3.86 (m, 1H), 2.46-2.22 (m, 2H), 2.18-2.06 (m, 1H), 2.02-1.90 (m, 1H), 1.85-1.64 (m, 2H), 1.64-1.47 (m, 3H), 1.46-1.28 (m, 4H), 0.91 (m, 3H). ³¹P NMR (162 MHz, chloroform-d) δ −2.68, −2.79. LCMS: MS m/z=563.1 [M+1]; 561.3 [M−1], t_{R}=1.62 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=4.58 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

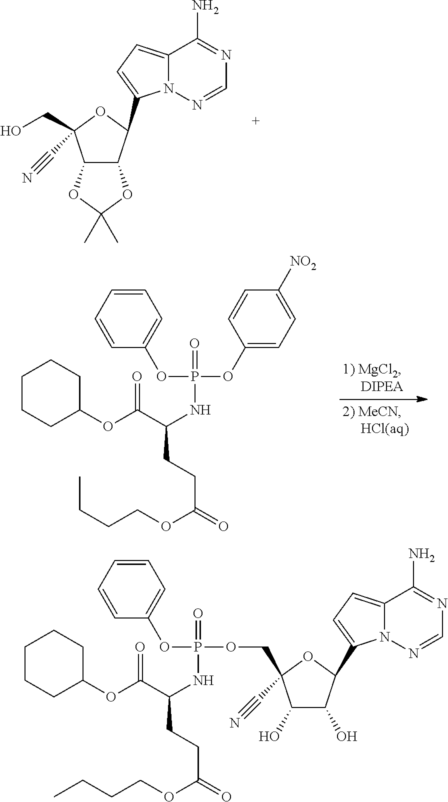

5-butyl 1-cyclohexyl ((((2R,3S,4R,5S)-5-(4-aminopyrrolo [2,1-f][1,2,4]triazin-7-yl)-2-cyano-3,4-dihydroxytetrahydrofuran-2-yl)methoxy)(phenoxy) phosphoryl)-L-glutamate. Intermediate 4 (50 mg, 0.15 mmol) and 5-butyl 1-cyclohexyl ((4-nitrophenoxy)(phenoxy)phosphoryl)-L-glutamate (110 mg, 0.195 mmol) were mixed and dissolved in 1.5 mL of anhydrous tetrahydrofuran. Magnesium chloride (43 mg, 0.45 mmol) was added in one portion. DIPEA (131 μL, 0.75 mmol) was added and the reaction was stirred at 60° C. for 16 h.

More 5-butyl 1-cyclohexyl ((4-nitrophenoxy)(phenoxy)phosphoryl)-L-glutamate (30 mg) and DIPEA (52 μL) were added. The reaction mixture was stirred at 60° C. for 6 h, and the reaction was then diluted with ethyl acetate (10 mL) and washed with water (5×10 mL) and then with brine (5 mL). The organic extract was dried over anhydrous sodium sulfate and concentrated under reduced pressure. The resulting material was dissolved in 5 mL of MeCN and stirred in an ice bath. Concentrate HCl(aq) (300 μL) was added dropwise and then stirred in an ice bath for 2 h. The reaction was diluted with ethyl acetate (10 mL) and washed with saturated aqueous sodium bicarbonate solution (10 mL) and then with brine (10 mL). The organic layer was dried over anhydrous sodium sulfate and concentrated under reduced pressure. The crude residue was purified via SiO₂ column chromatography (4 g SiO₂ Combiflash HP Gold Column, 0-10% methanol/dichloromethane). Fractions containing the desired product were combined and concentrated under reduced pressure. The resulting material was re-purified with prep-HPLC without acid modifier (5-100% MeCN/water). Fractions containing the desired product were combined and freeze-dried to afford the product. ¹H NMR (400 MHz, methanol-d₄) δ 7.79 (m, 1H), 7.37-7.10 (m, 5H), 6.84 (m, 1H), 6.73 (m, 1H), 5.50 (m, 1H), 4.76-4.55 (m, 2H), 4.52-4.30 (m, 3H), 4.07-3.81 (m, 3H), 2.45-2.20 (m, 2H), 2.03 (m, 1H), 1.88-1.62 (m, 5H), 1.62-1.18 (m, 10H), 0.90 (m, 3H). ³¹P NMR (162 MHz, methanol-d₄) δ 3.44, 3.47. LCMS: MS m/z=715.2 [M+1]; 713.2 [M−1], t_{R}=1.35 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.28 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=5.528 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 230. ethyl 2-(((4aR,6S,7S,7aS)-6-(4-aminopyrrolo[2,1-f][1,2,4]triazin-7-yl)-4a-cyano-7-ethoxy-2-oxidotetrahydro-4H-furo[3,2-d][1,3,2]dioxaphosphinin-2-yl)oxy)benzoate

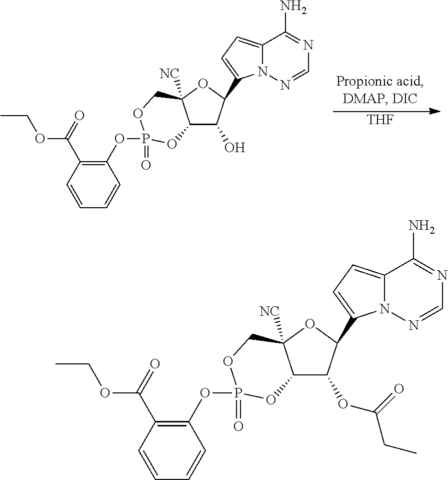

Dissolved Example 224 (13 mg, 0.026 mmol) in 1 mL THF, to the solution were added propionic acid (12 mg, 0.16 mmol) and DIC (13 mg, 0.1 mmol). The reaction mixture was stirred for 10 mins at RT, then DMAP (9 mg, 0.07 mmol) was added. The resulting mixture was stirred at RT for 30 mins and then solvent was evaporated. The residue was purified with Prep HPLC to afford the product. ¹H NMR (400 MHz, Methanol-d4) δ 8.02 (dt, J=7.9, 1.5 Hz, 1H), 7.89 (s, 1H), 7.66 (ddd, J=8.9, 7.3, 1.8 Hz, 1H), 7.59 (dt, J=8.3, 1.2 Hz, 1H), 7.43-7.32 (m, 1H), 7.07 (d, J=4.7 Hz, 1H), 6.98 (d, J=4.6 Hz, 1H), 5.95 (dd, J=5.7, 2.6 Hz, 1H), 5.92-5.85 (m, 2H), 5.01-4.86 (m, 2H), 4.38 (q, J=7.1 Hz, 2H), 2.52 (q, J=7.5 Hz, 2H), 1.39 (t, J=7.1 Hz, 3H), 1.20 (t, J=7.6 Hz, 3H). ³¹P NMR (162 MHz, Methanol-d4) δ −14.26. LCMS: MS m/z=558.0 4 [M+1], t_{R}=1.29 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.97 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 231. Cyclohexyl ((S)-(((2R,3S,4R,5S)-5-(4-butyramidopyrrolo[2,1-f][1,2,4]triazin-7-yl)-2-cyano-3,4-dihydroxytetrahydrofuran-2-yl)methoxy)(phenoxy)phosphoryl)-L-alaninate

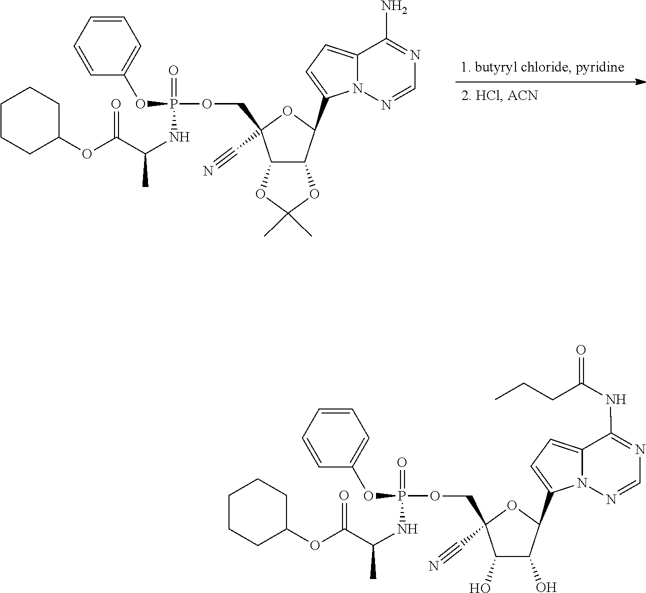

To a solution of Example 211 (44 mg, 0.069 mmol) in DCM (2 mL) were added pyridine (0.055 mL, 0.609 mmol) and butyryl chloride (0.014 mL, 0.137 mmol) sequentially. The resulting mixture was stirred at room temperature for 2 h and quenched by adding methanol (0.1 mL), diluted with EtOAc, washed with brine, and dried with sodium sulfate, concentrated in vacuo, and co-evaporated with toluene several time. The resulting residue was dissolved in acetonitrile (2 mL) and c-HCl (0.1 mL) added. The resulting mixture was stirred at room temperature for 1 h and aq. NaHCO₃ (2 mL) added under ice bath. The mixture was then purified by preparative HPLC (Phenomenex Gemini-NX 10μ C18 110° A 250×30 mm column, 5%-100% acetonitrile/water gradient in 25 min run) to afford the product. ¹H NMR (400 MHz, Acetonitrile-d3) δ 8.91 (s, 1H), 8.17 (s, 1H), 7.35 (dd, J=8.6, 7.2 Hz, 2H), 7.27-7.17 (m, 3H), 7.15 (d, J=4.7 Hz, 1H), 6.91 (d, J=4.7 Hz, 1H), 5.56 (d, J=4.6 Hz, 1H), 4.65 (td, J=8.5, 3.9 Hz, 1H), 4.57 (t, J=4.9 Hz, 1H), 4.48 (d, J=6.2 Hz, 2H), 4.44-4.29 (m, 3H), 4.13 (s, 1H), 3.90 (tq, J=9.7, 7.1 Hz, 1H), 2.66 (t, J=7.4 Hz, 2H), 1.70 (qd, J=13.0, 11.3, 7.4 Hz, 6H), 1.54-1.45 (m, 1H), 1.44-1.18 (m, 8H), 1.00 (t, J=7.4 Hz, 3H). ³¹P NMR (162 MHz, Acetonitrile-d3) δ 2.81. LCMS: MS m/z=671.32 [M+1]; t_{R}=1.09 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.48 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 232. Cyclohexyl ((S)-(((2R,3S,4R,5S)-2-cyano-5-(4-dodecanamidopyrrolo[2,1-f][1,2,4]triazin-7-yl)-3,4-dihydroxytetrahydrofuran-2-yl)methoxy)(phenoxy)phosphoryl)-L-alaninate

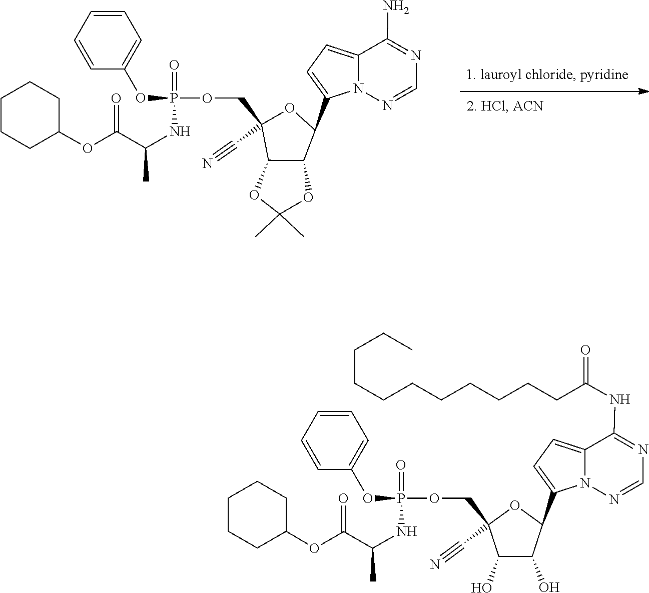

To a solution of Example 211 (66 mg, 0.103 mmol) in DCM (2 mL) were added pyridine (0.2 mL, 2.483 mmol) and lauroyl chloride (0.036 mL, 0.155 mmol) sequentially. The resulting mixture was stirred at room temperature for 3 h and quenched by adding methanol (0.1 mL), diluted with EtOAc, washed with water, and dried with sodium sulfate, concentrated in vacuo, dissolved in ACN (2 mL), and c-HCl (0.1 mL) added. The resulting mixture was stirred at room temperature for 2 h, and aq. NaHCO₃ (2 mL) added under ice bath. The reaction mixture was then purified by preparative HPLC (Phenomenex Gemini-NX 10μ C18 110° A 250×30 mm column, 5%-100% acetonitrile/water gradient in 25 min run) to afford the product. ¹H NMR (400 MHz, Acetonitrile-d3) δ 8.88 (s, 1H), 8.16 (s, 1H), 7.35 (dd, J=8.6, 7.2 Hz, 2H), 7.27-7.18 (m, 3H), 7.15 (d, J=4.7 Hz, 1H), 6.91 (d, J=4.7 Hz, 1H), 5.55 (d, J=4.6 Hz, 1H), 4.66 (dt, J=8.7, 4.5 Hz, 1H), 4.57 (t, J=5.1 Hz, 1H), 4.47 (d, J=5.7 Hz, 1H), 4.41 (dd, J=11.2, 6.9 Hz, 1H), 4.37-4.29 (m, 2H), 4.07 (s, 1H), 3.90 (tq, J=9.7, 7.1 Hz, 1H), 2.67 (t, J=7.4 Hz, 2H), 1.83-1.60 (m, 6H), 1.54-1.44 (m, 1H), 1.45-1.21 (m, 24H), 0.95-0.83 (m, 3H). ³¹P NMR (162 MHz, Acetonitrile-d3) δ 2.80. LCMS: MS m/z=783.46 [M+1]; t_{R}=1.56 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=7.56 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 233. ethyl ((S)-(((3aS,4R,6S,6aS)-6-(4-aminopyrrolo[2,1-f][1,2,4]triazin-7-yl)-4-cyano-2-ethoxytetrahydrofuro[3,4-d][1,3]dioxol-4-yl)methoxy)(phenoxy)phosphoryl)-L-alaninate

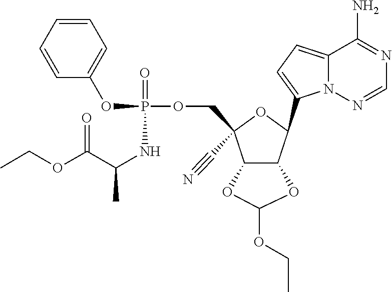

Example 29 (34 mg, 0.063 mmol) was dissolved in DMF (3 mL), and p-toluenesulfonic acid (21 mg, 0.12 mmol) and the ortho ester (461 mg, 3.1 mmol) was added under argon at RT. The solution was stirred for 40 min, diluted with AcOEt (50 mL), washed with brine (4×20 mL), sat. aqueous NaHCO₃ (20 mL), and the solvent evaporated under vacuum. The residue was purified with prep HPLC to afford the product. ¹H NMR (400 MHz, Methanol-d4) δ 7.81 (s, 1H), 7.39-7.27 (m, 2H), 7.27-7.13 (m, 3H), 6.86 (d, J=4.5 Hz, 1H), 6.77 (d, J=4.5 Hz, 1H), 6.12 (s, 1H), 5.83 (d, J=5.2 Hz, 1H), 5.44 (dd, J=7.2, 5.2 Hz, 1H), 5.03 (d, J=7.2 Hz, 1H), 4.40 (dd, J=10.4, 6.1 Hz, 1H), 4.32 (dd, J=10.4, 5.3 Hz, 1H), 4.10 (qt, J=7.0, 3.6 Hz, 3H), 3.96-3.84 (m, 2H), 3.73 (dq, J=9.3, 7.0 Hz, 1H), 1.29 (td, J=6.5, 5.0 Hz, 6H), 1.24-1.11 (m, 3H). ³¹P NMR (162 MHz, Methanol-d4) δ 3.18. LCMS: MS m/z=603.00 [M+1], t_{R}=1.27 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.951 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 234. isopropyl ((S)-(((2R,3S,4R,5S)-2-cyano-3,4-dihydroxy-5-(4-isobutyramidopyrrolo[2,1-f][1,2,4]triazin-7-yl)tetrahydrofuran-2-yl)methoxy)(phenoxy) phosphoryl)-L-alaninate

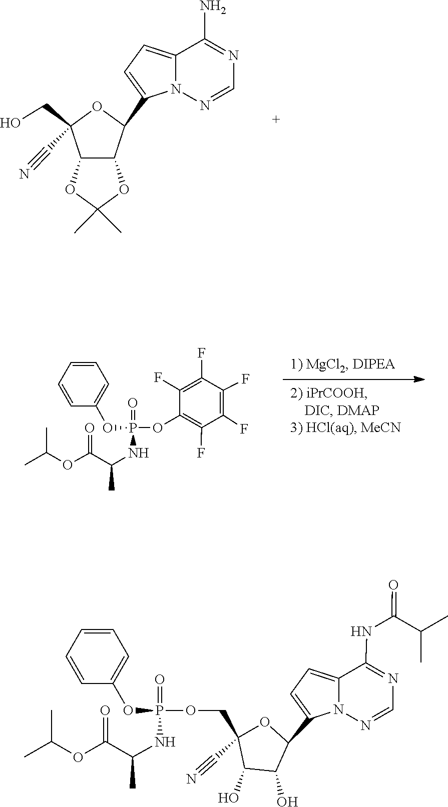

Intermediate 4 (100 mg, 0.3 mmol) and isopropyl ((S)-(perfluorophenoxy)(phenoxy)phosphoryl)-L-alaninate (prepared according to WO2011123672, 150 mg, 0.33 mmol) were mixed and dissolved in 3 mL of anhydrous tetrahydrofuran. Magnesium chloride (114 mg, 1.2 mmol) was added in one portion. DIPEA (130 μL, 0.75 mmol) was added and the reaction was stirred at 50° C. for 6 h. More isopropyl ((S)-(perfluorophenoxy)(phenoxy)phosphoryl)-L-alaninate (50 mg) was added and the reaction was stirred at 50° C. for 16 h. The reaction mixture was diluted with ethyl acetate (15 mL) and washed with aqueous sodium carbonate solution (2×10 mL) and then with brine (10 mL). The organic extract was dried over anhydrous sodium sulfate and concentrated under reduced pressure. The crude residue was purified via SiO₂ column chromatography (4 g SiO₂ Combiflash HP Gold Column, 0-100% ethyl acetate/hexanes). Fractions containing the desired product were combined and concentrated under reduced pressure. The resulting intermediate (60 mg, 0.1 mmol) was dissolved in 2 mL of anhydrous tetrahydrofuran. Isobutyric acid (14 μL, 0.15 mmol) and N,N′-diisopropylcarbodiimide (23 μL, 0.15 mmol) were added and the reaction mixture was stirred for 30 min. DMAP (12 mg, 0.1 mmol) was added and the mixture was stirred for 2 h. The reaction mixture was then heated to 50° C. and stirred for 3 h. More isobutyric acid (14 μL), N,N′-diisopropylcarbodiimide (23 μL) and DMAP (12 mg) were added. The reaction mixture was then stirred at 50° C. for 18 h. Methanol (2 mL) was then added and resulting mixture was stirred for 40 min. The reaction mixture was diluted with ethyl acetate (15 mL) and washed with saturated aqueous sodium bicarbonate (2×10 mL) followed with brine (2×5 mL). The organic extract was dried over anhydrous sodium sulfate and concentrated under reduced pressure. The crude residue was purified via SiO₂ column chromatography (4 g SiO₂ Combiflash HP Gold Column, 0-60% ethyl acetate/hexanes). The fractions containing the desired product were combined and concentrated under reduced pressure. The result material was dissolved in MeCN (5 mL) and stirred in an ice bath. Concentrated HCl (aq) (300 μL) was added dropwise and was stirred for 3 h in an ice bath. The reaction mixture was then diluted with ethyl acetate (10 mL) and washed with saturated aqueous sodium bicarbonate (2×10 mL) followed with brine (5 mL). The organic extract was dried over anhydrous sodium sulfate and concentrated under reduced pressure. The crude residue was purified via SiO₂ column chromatography (4 g SiO₂ Combiflash HP Gold Column, 0-100% ethyl acetate/hexanes). The fractions containing the desired product were combined and concentrated under reduced pressure which was dissolved in MeCN and water and freeze-dried to give the product. ¹H NMR (400 MHz, methanol-d₄) δ 8.18 (s, 1H), 7.37-7.26 (m, 2H), 7.26-7.11 (m, 4H), 6.96 (d, J=4.7 Hz, 1H), 5.59 (d, J=5.0 Hz, 1H), 4.93-4.84 (m, 1H), 4.61 (t, J=5.3 Hz, 1H), 4.50-4.31 (m, 3H), 3.92-3.71 (m, 2H), 2.96 (p, J=6.8 Hz, 1H), 1.30-1.20 (m, 12H), 1.09 (d, J=6.5 Hz, 3H). ³¹P NMR (162 MHz, methanol-d₄) δ 3.32. LCMS: MS m/z=631.2 [M+1]; 629.4 [M−1], t_{R}=1.24 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.01 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=4.900 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 235. cyclohexyl ((S)-(((2R,3S,4R,5S)-2-cyano-3,4-dihydroxy-5-(4-isobutyramidopyrrolo[2,1-f][1,2,4]triazin-7-yl)tetrahydrofuran-2-yl)methoxy)(phenoxy) phosphoryl)-L-alaninate

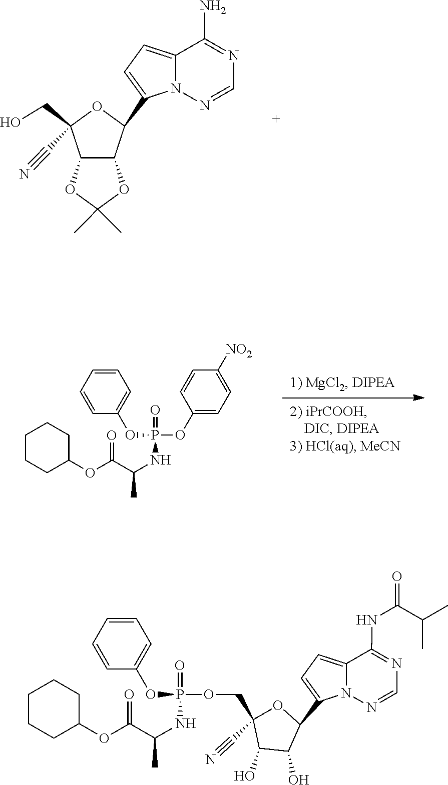

Intermediate 4 (100 mg, 0.3 mmol) and Intermediate 74 (161 mg, 0.36 mmol) were mixed and dissolve in 3 mL of anhydrous tetrahydrofuran. Magnesium chloride (114 mg, 1.2 mmol) was added in one portion. DIPEA (130 μL, 0.75 mmol) was added, and the reaction was stirred at 50° C. for 18 h. The reaction was diluted with ethyl acetate (15 mL) and washed with water (2×20 mL), aqueous sodium carbonate solution (2×20 mL) and then with brine (10 mL). The organic extract was dried over anhydrous sodium sulfate and concentrated under reduced pressure. The crude residue was purified via SiO₂ column chromatography (4 g SiO₂ Combiflash HP Gold Column, 0-100% ethyl acetate/hexanes). Fractions containing the desired product were combined and concentrated under reduced pressure. The resulting intermediate (55 mg, 0.086 mmol) was dissolved in 1 mL of anhydrous tetrahydrofuran. Isobutyric acid (24 μL, 0.258 mmol) and N,N′-diisopropylcarbodiimide (40 μL, 0.258 mmol) were added and the reaction mixture was stirred for 30 min. DIPEA (45 μL, 0.258 mmol) was added and the reaction mixture was stirred at 50° C. for 72 h. Methanol (2 mL) was added and stirred for 40 min. The resulting mixture was diluted with ethyl acetate (15 mL) and washed with saturated aqueous sodium bicarbonate (2×10 mL) followed with brine (2×10 mL). The organic extract was dried over anhydrous sodium sulfate and concentrated under reduced pressure. The crude residue was purified via SiO₂ column chromatography (4 g SiO₂ Combiflash HP Gold Column, 0-70% ethyl acetate/hexanes). The fractions containing the desired product were combined and concentrated under reduced pressure and then dissolved in MeCN (5 mL) and stirred in an ice bath. Concentrate HCl (aq) (300 μL) was then added dropwise, and the reaction mixture was stirred for 3 h in an ice bath. The reaction mixture was then diluted with ethyl acetate (10 mL) and washed with saturated aqueous sodium bicarbonate (2×10 mL) followed with brine (5 mL). Organic extract was dried over anhydrous sodium sulfate and concentrated under reduced pressure. The crude residue was purified via SiO₂ column chromatography (4 g SiO₂ Combiflash HP Gold Column, 0-80% ethyl acetate/hexanes). The fractions containing the desired product were combined and concentrated under reduced pressure as oil, which was dissolved in MeCN and water and freeze-dried to give the product. ¹H NMR (400 MHz, methanol-d₄) δ 8.18 (s, 1H), 7.37-7.27 (m, 2H), 7.26-7.11 (m, 4H), 6.96 (d, J=4.7 Hz, 1H), 5.59 (d, J=5.0 Hz, 1H), 4.70-4.57 (m, 2H), 4.47-4.31 (m, 3H), 3.93-3.71 (m, 2H), 2.96 (p, J=6.8 Hz, 1H), 1.82-1.56 (m, 2H), 1.55-1.44 (m, 1H), 1.43-1.18 (m, 10H), 1.09 (d, J=6.5 Hz, 6H). ³¹P NMR (162 MHz, methanol-d₄) δ 3.31. LCMS: MS m/z=671.3 [M+1]; 669.4 [M−, 1], t_{R}=1.34 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.34 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=5.465 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 236. isopropyl ((S)-(((3aS,4R,6S,6aS)-6-(4-aminopyrrolo[2,1-f][1,2,4]triazin-7-yl)-4-cyano-2-oxotetrahydrofuro[3,4-d][1,3]dioxol-4-yl)methoxy) (phenoxy)phosphoryl)-L-alaninate

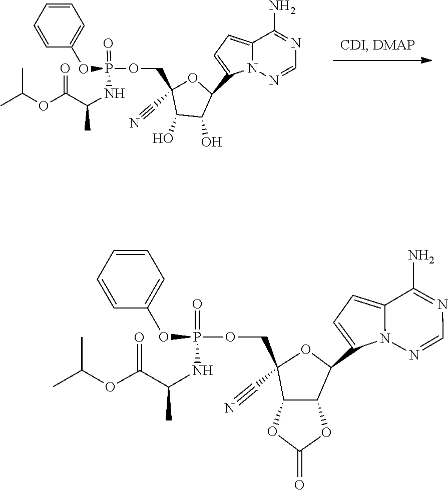

Example 1 (55 mg, 0.1 mmol) was dissolved in 3 mL of anhydrous tetrahydrofuran. 1,1′-carbonyl-diimidazole (24 mg, 0.15 mmol) was added in one portion. DMAP (2.4 mg, 0.02 mmol) was added and the reaction mixture was stirred for 20 h. The reaction mixture was diluted with ethyl acetate (15 mL) and washed with brine (2×10 mL). The organic extract was dried over anhydrous sodium sulfate and concentrated under reduced pressure. The crude residue was purified via SiO₂ column chromatography (4 g SiO₂ Combiflash HP Gold Column, 0-100% ethyl acetate/hexanes). The fractions containing the desired product were combined and concentrated under reduced pressure. Material was dissolved in MeCN and water and freeze-dried to give the product. ¹H NMR (400 MHz, chloroform-d) δ 7.83 (s, 1H), 7.39-7.27 (m, 2H), 7.27-7.12 (m, 3H), 6.69 (d, J=4.6 Hz, 1H), 6.60 (d, J=4.5 Hz, 1H), 5.68-5.57 (m, 2H), 5.53-5.44 (m, 1H), 4.99 (p, J=6.3 Hz, 1H), 4.50-4.33 (m, 3H), 4.07-3.91 (m, 1H), 1.36 (d, J=7.1 Hz, 3H), 1.24-1.19 (m, 6H). ³¹P NMR (162 MHz, chloroform-d) δ 2.67. LCMS: MS m/z=587.1 [M+1]; 585.3 [M−1], t_{R}=1.26 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=2.99 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=4.971 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 237. cyclohexyl ((S)-(((2R,3S,4R,5S)-5-(4-((S)-2-amino-3-methylbutanamido)pyrrolo[2,1-f][1,2,4]triazin-7-yl)-2-cyano-3,4-dihydroxy tetrahydrofuran-2-yl)methoxy)(phenoxy)phosphoryl)-L-alaninate

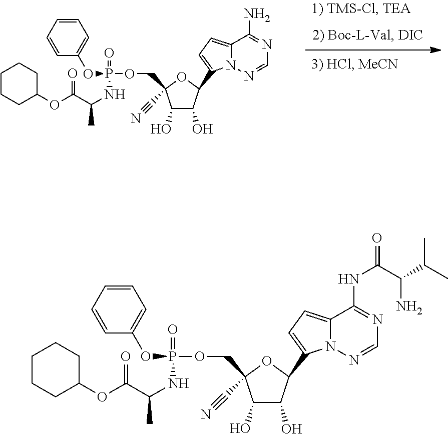

Example 6 (100 mg, 0.166 mmol) was dissolved in 5 mL of anhydrous tetrahydrofuran. Triethylamine (24 μL, 0.5 mmol) and TMSCl (63 μL, 0.5 mmol) were added, and the reaction mixture was stirred for 1 h. More triethylamine (70 μL) and TMSCl (60 μL) were added and the reaction mixture was stirred for 16 h. The reaction was concentrated under reduced pressure, and the resulting material was mixed with anhydrous tetrahydrofuran (3 mL) and stirred at RT.

Boc-L-Valine (54 mg, 0.249 mmol) and N,N′-diisopropylcarbodiimide (39 μL, 0.249 mmol) were mixed and dissolved in 1 mL of anhydrous tetrahydrofuran and stirred for 25 min. This mixture was then added to the above reaction. Triethylamine (70 μL) was added, and the reaction was stirred at RT for 2 h. Boc-L-Valine (54 mg, 0.249 mmol) and N,N′-diisopropylcarbodiimide (39 μL, 0.249 mmol) were mixed and dissolved in 1 mL of anhydrous tetrahydrofuran and stirred for 25 min. The resulting mixture was added to the reaction. Triethylamine (70 μL) was added, and the reaction was stirred at RT for 2 h. DMAP (40 mg) was added and stirred at RT for 16 h.

Methanol (2 mL) was added to the reaction and stirred for 60 min. The reaction was diluted with ethyl acetate (15 mL) and washed with saturated aqueous sodium bicarbonate (2×10 mL) followed with brine (2×5 mL). The organic extract was dried over anhydrous sodium sulfate and concentrated under reduced pressure. The crude residue was purified via SiO₂ column chromatography (4 g SiO₂ Combiflash HP Gold Column, 0-50% ethyl acetate/hexanes). Fractions containing the desired product were combined and concentrated under reduced pressure. The result material was dissolved in MeCN (5 mL) and stirred in an ice bath. Concentrated HCl (aq) (300 μL) was added dropwise. The reaction mixture was stirred for 2 h in an ice bath. More concentrate HCl (aq) (200 μL) was added dropwise and reaction was stirred for 2 h in an ice bath. Saturated aqueous bicarbonate solution was added to give pH of 8-9 and stirred for 15 min. The reaction mixture was diluted with ethyl acetate (15 mL) and washed with saturated aqueous sodium bicarbonate (2×10 mL) followed with brine (5 mL). The organic extract was dried over anhydrous sodium sulfate and concentrated under reduced pressure. The crude residue was purified via SiO₂ column chromatography (4 g SiO₂ Combiflash HP Gold Column, 0-5% methanol/dichloromethane). Fractions containing the desired product were combined and concentrated under reduced pressure. The result material was purified with prep HPLC (5-98% MeCN/water) using 0.1% TFA as modifier. Fractions containing the desired product were combined, neutralized with saturated sodium bicarbonate solution and extracted with ethyl acetate (15 mL). The organic extract was washed with brine (5 mL), dried over anhydrous sulfate and concentrated under reduced pressure. The residue was purified via SiO₂ column chromatography (4 g SiO₂ Combiflash HP Gold Column, 0-5% methanol/dichloromethane). Fractions containing the desired product were combined and concentrated under reduced pressure. The residue was dissolved in MeCN and water and freeze-dried to give the product. ¹H NMR (400 MHz, methanol-d₄) δ 7.87 (s, 1H), 7.36-7.28 (m, 2H), 7.25-7.13 (m, 3H), 6.96 (d, J=4.5 Hz, 1H), 6.73 (d, J=4.5 Hz, 1H), 5.53-5.49 (m, 1H), 4.69-4.57 (m, 3H), 4.50-4.38 (m, 2H), 4.34 (m, 1H), 3.88 (m, 1H), 2.24 (m, 1H), 1.82-1.59 (m, 4H), 1.53-1.43 (m, 1H), 1.43-1.17 (m, 8H), 1.04 (m, 6H). ³¹P NMR (162 MHz, methanol-d₄) δ 3.29, 3.30. LCMS: MS m/z=700.2 [M+1]; 698.4 [M−1], t_{R}=1.28 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 m/min. HPLC: t_{R}=3.31 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=5.282, 5.332 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 238. (2S,3R,4S,5R)-2-(4-aminopyrrolo[2,1-f][1,2,4]triazin-7-yl)-5-cyano-4-hydroxy-5-((((S)—(((S)-1-isopropoxy-1-oxopropan-2-yl)amino)(phenoxy)phosphoryl)oxy)methyl)tetrahydrofuran-3-yl pivalate

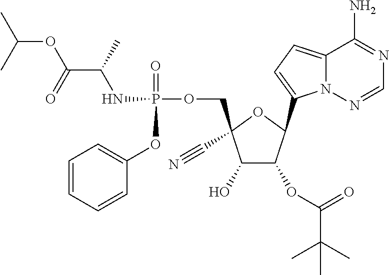 [SMILES: CC(C)OC(=O)[C@H](C)N[P@](=O)(OC[C@@]1(C#N)O[C@@H](c2ccc3c(N)ncnn23)[C@H](OC(=O)C(C)(C)C)[C@@H]1O)Oc1ccccc1]

N,N′-Diisopropylcarbodiimide (0.11 mL, 0.71 mmol) and 4-dimethylaminopyridine (89.4 mg, 0.73 mmol) were added to a solution of Example 1 (41.3 mg, 0.07 mmol) and pivalic acid (76.5 mL, 0.75 mmol) in tetrahydrofuran (6 mL) at RT. After 20 h at 60° C., methanol (0.5 mL) was added, and the reaction mixture was diluted with ethyl acetate (20 mL) and the resulting mixture was washed with water (15 mL) and brine (15 mL). The organic layer was dried over anhydrous sodium sulfate and concentrated under reduced pressure. The crude residue was subjected preparatory HPLC (Phenomenex Gemini 10 u C18 110 Å AXIA 250×21.2 mm column, 30-70% acetonitrile/water gradient with 0.1% TFA) to afford the products as a TFA salt.

First Eluting Example: ¹H NMR (400 MHz, acetonitrile-d₃) δ 7.91 (s, 1H), 7.40-7.29 (m, 2H), 7.23-7.13 (m, 4H), 6.88 (d, J=4.7 Hz, 1H), 5.61 (d, J=3.4 Hz, 1H), 5.53 (dd, J=5.9, 3.4 Hz, 1H), 4.88 (p, J=6.2 Hz, 1H), 4.75 (d, J=5.8 Hz, 1H), 4.43 (dd, J=11.4, 7.3 Hz, 1H), 4.34 (dd, J=11.4, 7.0 Hz, 1H), 4.25 (t, J=11.0 Hz, 1H), 3.94-3.74 (m, 1H), 1.27 (d, J=7.0 Hz, 3H), 1.24 (s, 9H), 1.16 (dd, J=6.3, 4.3 Hz, 6H). ¹⁹F NMR (376 MHz, acetonitrile-d₃) δ −77.08. ³¹P NMR (162 MHz, acetonitrile-d₃) δ 2.88. LCMS: MS m/z=645.41 [M+1], t_{R}=0.98 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.54 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Example 239. (2R,3S,4S,5S)-5-(4-aminopyrrolo[2,1-f][1,2,4]triazin-7-yl)-2-cyano-4-hydroxy-2-((((S)—(((S)-1-isopropoxy-1-oxopropan-2-yl)amino)(phenoxy)phosphoryl)oxy)methyl)tetrahydrofuran-3-yl pivalate

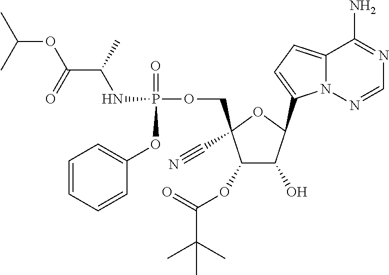 [SMILES: CC(C)OC(=O)[C@H](C)N[P@](=O)(OC[C@@]1(C#N)O[C@@H](c2ccc3c(N)ncnn23)[C@H](O)[C@@H]1OC(=O)C(C)(C)C)Oc1ccccc1]

Second Eluting Example of Example 238: ¹H NMR (400 MHz, acetonitrile-d₃) δ 7.95 (s, 1H), 7.39-7.31 (m, 2H), 7.25-7.13 (m, 4H), 6.91 (d, J=4.9 Hz, 1H), 5.49 (d, J=6.7 Hz, 1H), 5.40 (d, J=5.7 Hz, 1H), 4.90 (p, J=6.3 Hz, 1H), 4.75 (t, J=6.2 Hz, 1H), 4.46-4.30 (m, 2H), 4.24 (t, J=11.0 Hz, 1H), 3.94-3.78 (m, 1H), 1.29 (s, 9H), 1.27 (d, J=7.0 Hz, 4H), 1.17 (dd, J=6.3, 1.4 Hz, 7H). ¹⁹F NMR (376 MHz, acetonitrile-d₃) δ −77.06. ³¹P NMR (162 MHz, acetonitrile-d₃) δ 2.55. LCMS: MS m/z=645.38 [M+1], t_{R}=0.98 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.62 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 mL/min.

### Example 240. (2S,3R,4S,5R)-2-(4-aminopyrrolo[2,1-f][1,2,4]triazin-7-yl)-5-cyano-4-hydroxy-5-((((S)—(((S)-1-isopropoxy-1-oxopropan-2-yl)amino)(phenoxy)phosphoryl)oxy)methyl)tetrahydrofuran-3-yl isobutyrate

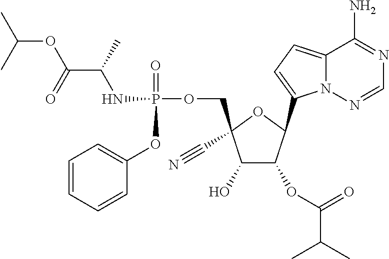 [SMILES: CC(C)OC(=O)[C@H](C)N[P@](=O)(OC[C@@]1(C#N)O[C@@H](c2ccc3c(N)ncnn23)[C@H](OC(=O)C(C)C)[C@@H]1O)Oc1ccccc1]

N,N′-diisopropylcarbodiimide (0.037 mL, 0.27 mmol) and 4-dimethylaminopyridine (33 mg, 0.27 mmol) were added to a solution of Example 1 (150 mg, 0.07 mmol) and isobutyric acid (24 mg, 0.27 mmol) in tetrahydrofuran (6 mL) at RT. After 20 h at RT, methanol (0.5 mL) was added, and the reaction mixture was diluted with ethyl acetate (20 mL) and the resulting mixture was washed with water (15 mL) and brine (15 mL). The organic layer was dried over anhydrous sodium sulfate and concentrated under reduced pressure. The crude residue was subjected preparatory HPLC (Phenomenex Gemini 10 u C18 110 Å AXIA 250×21.2 mm column, 30-70% acetonitrile/water gradient with 0.1% TFA) to afford the products as a TFA salt.

First Eluting Example: ¹H NMR (400 MHz, Methanol-d₄) δ 8.03-7.96 (m, 1H), 7.38-7.12 (m, 5H), 6.92 (q, J=4.5 Hz, 1H), 5.66 (q, J=4.0 Hz, 1H), 5.55 (p, J=5.2 Hz, 1H), 4.83 (s, 11H), 4.72 (q, J=5.3 Hz, 1H), 4.50-4.34 (m, 2H), 3.83 (d, J=8.7 Hz, 1H), 2.69 (p, J=7.2 Hz, 1H), 1.31-1.12 (m, 13H). ³¹P NMR (162 MHz, Methanol-d₄) δ 3.28. LCMS: MS m/z=631.29 [M+1], t_{R}=0.96 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.02; HPLC system: Agilent 1100 series; Column: Phenomenex Kinetex C18, 2.6μ C18 110 A, 100×4.6 mm; Solvents: Acetonitrile with 0.1% TFA, Water with 0.1% TFA; Gradient: 0 min-8.5.0 min 2-98% ACN, 8.5 min-10.0 min 98% ACN at 1.5 mL/min.

### Example 241. (2R,3S,4S,5S)-5-(4-aminopyrrolo[2,1-f][1,2,4]triazin-7-yl)-2-cyano-4-hydroxy-2-((((S)—(((S)-1-isopropoxy-1-oxopropan-2-yl)amino)(phenoxy)phosphoryl)oxy)methyl)tetrahydrofuran-3-yl isobutyrate

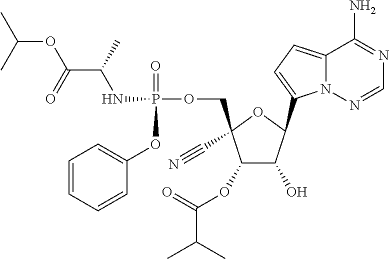 [SMILES: CC(C)OC(=O)[C@H](C)N[P@](=O)(OC[C@@]1(C#N)O[C@@H](c2ccc3c(N)ncnn23)[C@H](O)[C@@H]1OC(=O)C(C)C)Oc1ccccc1]

Second Eluting Example of Example 240: ¹H NMR (400 MHz, Methanol-d₄) δ 8.03 (s, 1H), 7.39-7.30 (m, 3H), 7.27-7.15 (m, 3H), 6.96 (d, J=4.7 Hz, 1H), 5.50 (dd, J=14.2, 6.3 Hz, 2H), 4.92 (dq, J=12.8, 6.4 Hz, 2H), 4.78 (dd, J=6.9, 5.7 Hz, 2H), 4.40 (d, J=6.1 Hz, 2H), 3.86 (dq, J=10.1, 7.1 Hz, 1H), 2.75 (h, J=7.0 Hz, 1H), 1.33-1.21 (m, 8H), 1.19 (dd, J=6.2, 0.9 Hz, 5H). ³¹P NMR (162 MHz, Methanol-d₄) δ 3.20. LCMS: MS m/z=631.29 [M+1], t_{R}=0.96 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.13; HPLC system: Agilent 1100 series; Column: Phenomenex Kinetex C18, 2.6μ C18 110 A, 100×4.6 mm; Solvents: Acetonitrile with 0.1% TFA, Water with 0.1% TFA; Gradient: 0 min-8.5.0 min 2-98% ACN, 8.5 min-10.0 min 98% ACN at 1.5 mL/min.

### Example 242. isopropyl ((S)-(((2R,3S,4R,5S)-5-(4-aminopyrrolo[2,1-f][1,2,4]triazin-7-yl)-2-cyano-3-hydroxy-4-(propionyloxy)tetrahydrofuran-2-yl)methoxy)(phenoxy)phosphoryl)-L-alaninate

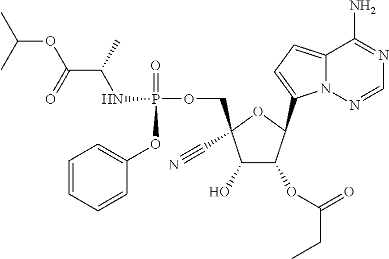 [SMILES: CCC(=O)O[C@H]1[C@H](c2ccc3c(N)ncnn23)O[C@](C#N)(CO[P@@](=O)(N[C@@H](C)C(=O)OC(C)C)Oc2ccccc2)[C@H]1O]

N,N′-diisopropylcarbodiimide (0.025 mL, 0.178 mmol) and 4-dimethylaminopyridine (22 mg, 0.178 mmol) were added to a solution of Example 1 (100 mg, 0.178 mmol) and propionic acid (13 mg, 0.178 mmol) in tetrahydrofuran (6 mL) at RT. After 20 h at RT, methanol (0.5 mL) was added, and the reaction mixture was diluted with ethyl acetate (20 mL) and the resulting mixture was washed with water (15 mL) and brine (15 mL). The organic layer was dried over anhydrous sodium sulfate and concentrated under reduced pressure. The crude residue was subjected preparatory HPLC (Phenomenex Gemini 10 u C18 110 Å AXIA 250×21.2 mm column, 30-70% acetonitrile/water gradient with 0.1% TFA) to afford the products as a TFA salt.

First Eluting Example: ¹H NMR (400 MHz, DMSO-d₆) δ 7.92 (s, 1H), 7.32 (dd, J=8.6, 7.2 Hz, 2H), 7.16 (dd, J=16.6, 7.9 Hz, 3H), 6.93 (d, J=4.6 Hz, 1H), 6.81 (d, J=4.5 Hz, 1H), 6.52 (s, 1H), 6.08 (dd, J=13.4, 10.1 Hz, 1H), 5.60-5.48 (m, 2H), 4.74 (p, J=6.1 Hz, 1H), 4.60 (s, 1H), 4.37 (dd, J=10.9, 6.3 Hz, 1H), 4.18 (dd, J=11.1, 4.5 Hz, 1H), 2.36 (q, J=7.5 Hz, 2H), 2.05 (s, 1H), 1.15 (d, J=7.1 Hz, 3H), 1.11-0.99 (m, 8H). ¹⁹F NMR (376 MHz, DMSO-d₆) δ −74.33. ³¹P NMR (162 MHz, DMSO-d₆) δ 3.29. LCMS: MS m/z=617.31 [M+1], t_{R}=0.89 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=7.14 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-19.0 min 2-95% ACN, 19.0 min-20.0 min 95% ACN at 2 mL/min.

### Example 243. isopropyl ((S)-(((2R,3S,4S,5S)-5-(4-aminopyrrolo[2,1-f][1,2,4]triazin-7-yl)-2-cyano-4-hydroxy-3-(propionyloxy)tetrahydrofuran-2-yl)methoxy)(phenoxy)phosphoryl)-L-alaninate

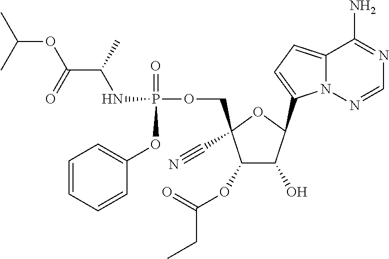 [SMILES: CCC(=O)O[C@H]1[C@@H](O)[C@H](c2ccc3c(N)ncnn23)O[C@]1(C#N)CO[P@@](=O)(N[C@@H](C)C(=O)OC(C)C)Oc1ccccc1]

Second Eluting Example of Example 242: ¹H NMR (400 MHz, Acetonitrile-d₃) δ 7.93 (d, J=9.2 Hz, 2H), 7.37 (dd, J=8.6, 7.1 Hz, 3H), 7.27-7.17 (m, 5H), 7.10-7.01 (m, 2H), 6.90-6.82 (m, 2H), 5.49 (dd, J=14.5, 5.9 Hz, 3H), 4.91 (p, J=6.3 Hz, 2H), 4.75 (q, J=6.5, 5.9 Hz, 2H), 4.47-4.22 (m, 6H), 2.54 (q, J=7.5 Hz, 11H), 1.28 (dt, J=7.4, 1.7 Hz, 5H), 1.23-1.09 (m, 15H), 1.02 (s, 1H). ³¹P NMR (162 MHz, acetonitrile-d₃) δ 2.56. LCMS: MS m/z=617.31 [M+1], t_{R}=0.89 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=7.31 min; HPLC system: Agilent 1100 series; Column: Gemini 5μ C18 110 A, 50×4.6 mm; Solvents: Acetonitrile with 0.1% TFA, Water with 0.1% TFA; Gradient: 0 min-19.0 min 2-95% ACN, 19.0 min-20.0 min 95% ACN at 2 mL/min.

### Example 244. 2-ethylbutyl (2S)-2-(((((2R,3S,4R,5S)-5-(4-aminopyrrolo [2,1-f][1,2,4]triazin-7-yl)-2-cyano-3,4-dihydroxytetrahydrofuran-2-yl)methoxy)(phenoxy) phosphoryl)oxy)propanoate

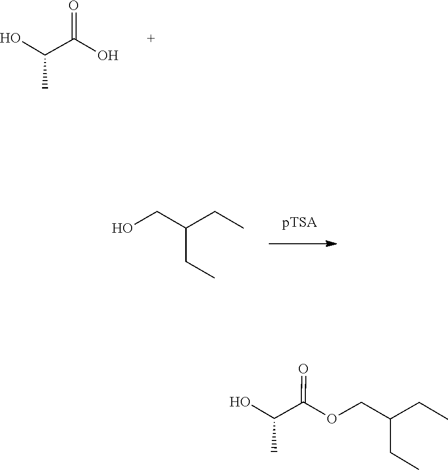

2-ethylbutyl (S)-2-hydroxypropanoate. 2-Ethylbutyl alcohol (1.8 mL, 15 mmol) was dissolved in 12 mL of benzene. p-Toluenesulfonic acid monohydrate (190 mg, 1.0 mmol) was added in one portion. The reaction mixture was stirred at reflux for 30 min. L-Lactic acid (900 mg, 10 mmol) was added in one portion and the reaction mixture was stirred at reflux for 16 h. The reaction mixture was then cooled and washed with saturated aqueous sodium bicarbonate (20 mL) followed with brine (10 mL). The organic extract was dried over anhydrous sodium sulfate and concentrated under reduced pressure. The crude residue was purified via SiO₂ column chromatography (4 g SiO₂ Combiflash HP Gold Column, 0-10% ethyl acetate/hexanes). Fractions containing the desired product were combined and concentrated under reduced pressure to give the intermediate. ¹H NMR (400 MHz, chloroform-d) δ 4.27 (q, J=7.5 Hz, 1H), 4.19-4.03 (m, 2H), 2.82 (bs, 1H), 1.55 (m, 1H), 1.41 (d, J=6.9 Hz, 3H), 1.39-1.31 (m, 4H), 0.90 (t, J=7.5 Hz, 6H).

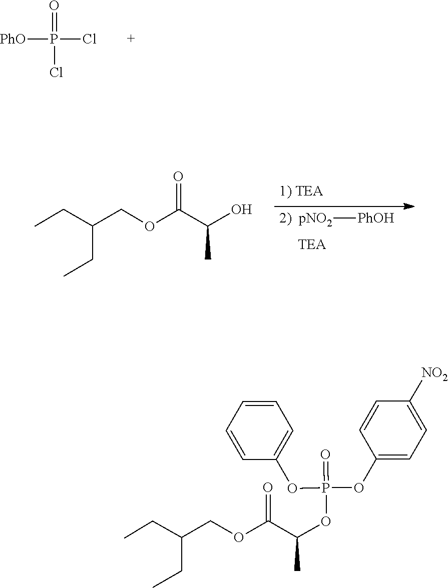

2-ethylbutyl (2S)-2-(((4-nitrophenoxy)(phenoxy)phosphoryl)oxy) propanoate. Phenyl dichlorophosphate (446 μL, 3 mmol) was dissolved in 10 mL anhydrous dichloromethane and stirred under atmospheric nitrogen in an ice bath. 2-ethylbutyl (S)-2-hydroxypropanoate (532 mg, 3 mmol) was dissolved in 3 mL anhydrous dichloromethane and added to the reaction solution dropwise. The reaction mixture was stirred for 1 h. Triethylamine (920 μL, 6.6 mmol) was dissolved in 1 mL anhydrous dichloromethane and added to the reaction dropwise. The reaction mixture was stirred for 16 h at RT. More triethylamine (460 μL, 3.3 mmol) was added to reaction and stirring continued for 3 h. p-Nitrophenol (376 mg, 2.7 mmol) was added in one portion, and the reaction mixture was stirred for 4 h. The reaction mixture was diluted with dichloromethane (15 mL) and washed with water (5×20 mL). The organic extract was dried over anhydrous sodium sulfate and concentrated under reduced pressure. The crude residue was purified via SiO₂ column chromatography (12 g SiO₂ Combiflash HP Gold Column, 0-15% ethyl acetate/hexanes). Fractions containing the desired product were combined and concentrated under reduced pressure to give the intermediate. ¹H NMR (400 MHz, chloroform-d) δ 8.31-8.18 (m, 2H), 7.50-7.29 (m, 4H), 7.29-7.11 (m, 3H), 5.18-5.04 (m, 1H), 4.18-3.98 (m, 2H), 1.65-1.45 (m, 4H), 1.40-1.28 (m, 4H), 0.93-0.82 (m, 6H). ³¹P NMR (162 MHz, chloroform-d) δ −13.26, −13.67. LCMS: MS m/z=452.0 [M+1], t_{R}=1.60 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=4.50 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min.

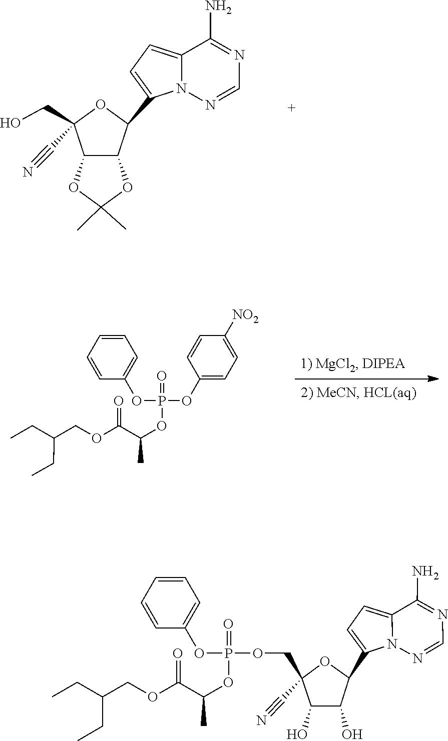 [SMILES: CC1(C)O[C@H]2[C@H](c3ccc4c(N)ncnn34)O[C@](C#N)(CO)[C@H]2O1 ; CCC(CC)COC(=O)[C@H](C)OP(=O)(Oc1ccccc1)Oc1ccc([N+](=O)[O-])cc1 ; CCC(CC)COC(=O)[C@H](C)OP(=O)(OC[C@@]1(C#N)O[C@@H](c2ccc3c(N)ncnn23)[C@H](O)[C@@H]1O)Oc1ccccc1]

2-Ethylbutyl (2S)-2-(((((2R,3S,4R,5S)-5-(4-aminopyrrolo [2,1-f][1,2,4]triazin-7-yl)-2-cyano-3,4-dihydroxytetrahydrofuran-2-yl)methoxy)(phenoxy) phosphoryl)oxy)propanoate. Intermediate 4 (50 mg, 0.15 mmol) and 2-ethylbutyl (2S)-2-(((4-nitrophenoxy)(phenoxy)phosphoryl)oxy) propanoate (81 mg, 0.18 mmol) were mixed and dissolved in 1.5 mL of anhydrous tetrahydrofuran. Magnesium chloride (43 mg, 0.45 mmol) was added in one portion. DIPEA (65 μL, 0.375 mmol) was added, and the reaction was stirred at 35° C. for 36 h.

The reaction mixture was diluted with ethyl acetate (10 mL) and washed with water (5×10 mL) and then with brine (5 mL). The organic extract was dried over anhydrous sodium sulfate and concentrated under reduced pressure. The residue was dissolved in MeCN (5 mL) and stirred in an ice bath. Concentrate aqueous hydrochloric acid (250 μL) was added dropwise. The reaction mixture was stirred in an ice bath for 2 h. The reaction mixture was then diluted with ethyl acetate (10 mL) and added saturated aqueous sodium bicarbonate solution (10 mL). The mixture was stirred for 10 min. The organic extract was washed with saturated aqueous sodium bicarbonate solution (10 mL) and then with brine (5 mL). The organic extract was dried over anhydrous sodium sulfate and concentrated under reduced pressure. The crude residue was purified via SiO₂ column chromatography (4 g SiO₂ Combiflash HP Gold Column, 0-5% methanol/dichloromethane). Fractions containing the desired product were combined and concentrated under reduced pressure. The residue was dissolved in MeCN and water and freeze-dried to give the product. ¹H NMR (400 MHz, methanol-d₄) δ 7.78 (m, 1H), 7.35-7.11 (m, 5H), 6.83 (m, 1H), 6.73 (m, 1H), 5.51 (m, 1H), 4.94 (m, 1H), 4.68-4.44 (m, 4H), 4.15-3.92 (m, 2H), 1.53-1.40 (m, 4H), 1.38-1.24 (m, 4H), 0.90-0.80 (m, 6H). ³¹P NMR (162 MHz, methanol-d₄) δ −8.40, −8.36. LCMS: MS m/z=604.2 [M+1], 602.5 [M−1], t_{R}=1.30 min; LC system: Thermo Dionex ultimate 3000 UHPLC; Column: Phenomenex Kinetex 2.6μ C18 100 A, 50×3 mm; Solvents: A: Water with 0.1% acetic acid, B: Acetonitrile with 0.1% acetic acid; Gradient: 0 min-0.3 min 5% B, 0.3 min-1.5 min 5-100% B, 1.5 min-2 min 100% B, 2 min-2.2 min 100-5% B at 2 mL/min. HPLC: t_{R}=3.12 min; HPLC system: Agilent 1100 series; Column: Phenomenex Gemini 5μ C18 110 A, 50×4.6 mm; Solvent: A: Water with 0.1% TFA, B: Acetonitrile with 0.1% TFA; Gradient: 2-98% B in 5 min at 2 mL/min. HPLC: t_{R}=5.267, 5.299 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 245. 3-hydroxy-2-(hydroxymethyl)propyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

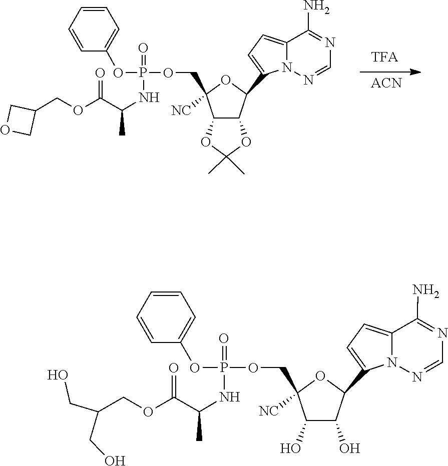

Dissolved Example 48 (100 mg, 0.16 mmol) in 3 mL ACN, mixed 8 mL of TFA with 1 mL water, then added the TFA solution to above reaction mixture, stirred at RT for 30 mins, quenched with aq. NaHCO₃ solution, extracted with EtOAc, evaporated organic solvent, purified with Prep HPLC to afford the product. ¹H NMR (400 MHz, Methanol-d4) δ 7.81 (d, J=5.5 Hz, 1H), 7.31 (ddd, J=13.8, 8.5, 7.0 Hz, 2H), 7.25-7.07 (m, 3H), 6.89 (t, J=4.2 Hz, 1H), 6.75 (dd, J=4.6, 0.9 Hz, 1H), 5.50 (dd, J=5.1, 2.8 Hz, 1H), 4.62 (dt, J=6.9, 5.3 Hz, 1H), 4.56-4.29 (m, 3H), 4.22-4.04 (m, 2H), 3.90 (dp, J=9.2, 7.1 Hz, 1H), 3.65-3.49 (m, 4H), 1.96 (dp, J=8.1, 5.9 Hz, 1H), 1.27 (dt, J=7.3, 1.4 Hz, 3H). ³¹P NMR (162 MHz, Methanol-d4) δ 3.33, 3.31. LCMS: MS m/z=607.10 [M+1], t_{R}=0.93 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=3.30 and 3.34 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 246. cyclohexylmethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

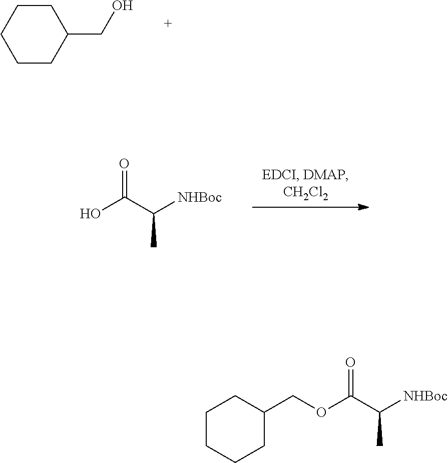 [SMILES: OCC1CCCCC1 ; C[C@H](NC(=O)OC(C)(C)C)C(=O)O ; C[C@H](NC(=O)OC(C)(C)C)C(=O)OCC1CCCCC1]

Cyclohexylmethyl (tert-butoxycarbonyl)-L-alaninate. Took up (tert-butoxycarbonyl)-L-alanine (7.954 g, 0.042 mol) in acetonitrile (90 mL) and cyclohexylmethanol (4.0 g, 0.035 mol) followed by EDCI (7.069 g, 0.046 mol) and DMAP (6.419 g, 0.053 mol) in one portion. Allowed to stir at room temperature for 4 h. Diluted with dichloromethane and water. The layers were split and the organic layer was dried over anhydrous sodium sulfate, filtered and was concentrated under reduced pressure. Purified by silica gel chromatography 0-50% ethyl acetate/hexane to afford the intermediate. ¹H NMR (400 MHz, DMSO-d6) δ 7.24 (d, J=7.5 Hz, 1H), 4.01-3.83 (m, 2H), 3.77 (dd, J=10.7, 6.3 Hz, 1H), 1.69-1.59 (m, 5H), 1.35 (s, 9H), 1.18 (dd, J=24.2, 8.9 Hz, 7H), 0.92 (q, J=11.5 Hz, 2H).

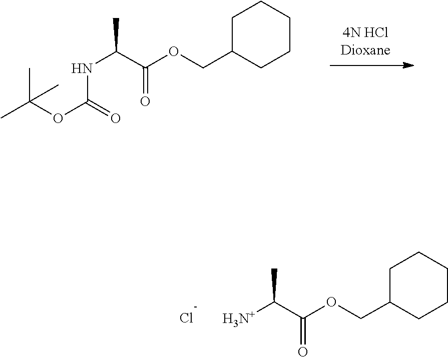 [SMILES: C[C@H](NC(=O)OC(C)(C)C)C(=O)OCC1CCCCC1 ; Cl ; C[C@H]([NH3+])C(=O)OCC1CCCCC1]

(S)-1-(Cyclohexylmethoxy)-1-oxopropan-2-aminium chloride. Took up cyclohexylmethyl (tert-butoxycarbonyl)-L-alaninate (6.5 g, 0.023 mol) in anhydrous dichloromethane (50 mL) and 4 N HCl in dioxane (17.08 mL, 0.068 mol). Stirred at ambient temperature for 4 h. Concentrated under reduced pressure and co-evaporated with dichloromethane. Placed under high vacuum for overnight and the intermediate was used as is without purification for the next step. ¹H NMR (400 MHz, DMSO-d₆) δ 8.55 (s, 3H), 4.10-3.88 (m, 3H), 1.71-1.53 (m, 5H), 1.40 (d, J=7.2 Hz, 3H), 1.27-1.07 (m, 4H), 0.95 (q, J=11.9 Hz, 2H).

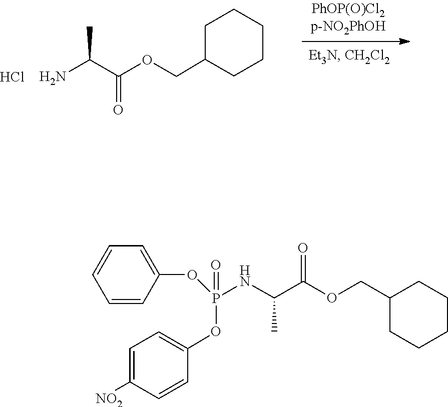 [SMILES: Cl ; C[C@H](N)C(=O)OCC1CCCCC1 ; C[C@H](NP(=O)(Oc1ccccc1)Oc1ccc([N+](=O)[O-])cc1)C(=O)OCC1CCCCC1]

Cyclohexylmethyl ((4-nitrophenoxy)(phenoxy)phosphoryl)-L-alaninate. To a solution of (S)-1-(cyclohexylmethoxy)-1-oxopropan-2-aminium chloride (2.0 g, 9.02 mmol) and phenyl dichlorophosphate (1.342 mL, 9.02 mmol) in anhydrous dichloromethane (35 mL) was added triethylamine (2.8 mL, 2.78 mmol) at 0° C. under argon atmosphere. The resulting mixture was stirred for 2 h at 0° C. 4-Nitrophenol (1.255 g, 9.02 mmol) and triethylamine (1.4 mL, 1.39 mmol) were then added. After 2 h stirring at 0° C., the reaction mixture was diluted with Et₂O and the solids were filtered off. The crude product was concentrated under reduced pressure and was purified by silica gel chromatography (80 g SiO₂ Combiflash HP Gold Column, 0-100% ethyl acetate/hexanes) to afford the intermediate. ¹H NMR (400 MHz, DMSO-d6) δ 8.32-8.25 (m, 2H), 7.53-7.34 (m, 4H), 7.29-7.16 (m, 3H), 6.68 (dt, J=13.7, 9.8 Hz, 1H), 4.06-3.91 (m, 1H), 3.78 (dd, J=6.5, 3.1 Hz, 2H), 1.59 (d, J=11.0 Hz, 5H), 1.25-0.97 (m, 7H), 0.87 (t, J=11.3 Hz, 2H). ³¹P NMR (162 MHz, DMSO-d6) δ −1.25, −1.50. LCMS: MS m/z=463.10 [M+1]; t_{R}=1.68 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

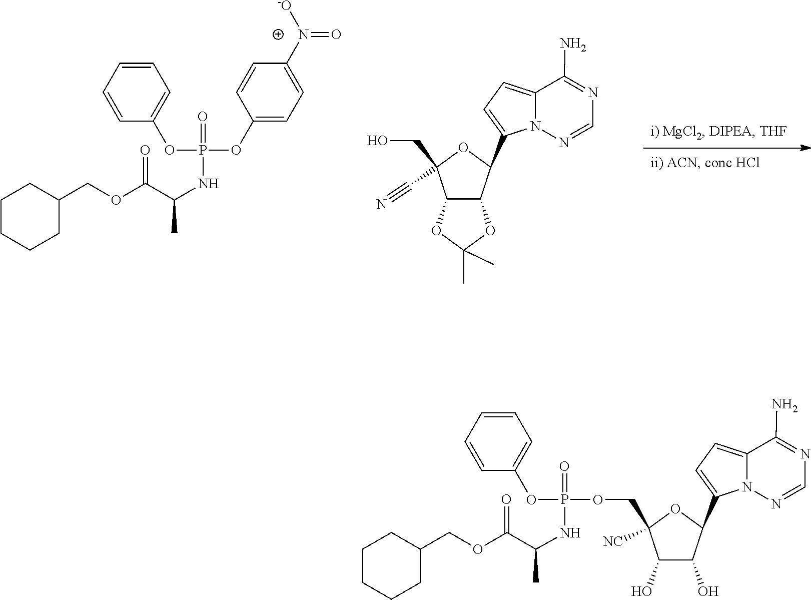

Cyclohexylmethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. To a mixture of Intermediate 4 (0.05 g, 0.151 mmol), intermediate cyclohexylmethyl ((4-nitrophenoxy)(phenoxy)phosphoryl)-L-alaninate (0.077 g, 0.166 mmol), and magnesium chloride (0.022 g, 0.226 mmol) was added tetrahydrofuran (1.5 mL) at room temperature followed by the addition of N,N-Diisopropylethylamine (0.049 mL, 0.377 mmol). The resulting mixture was stirred at 50° C. for 3 h. The reaction mixture was then concentrated under reduced pressure and the residue obtained was dissolved in an anhydrous acetonitrile (2 mL) and was cooled in an ice bath followed by the dropwise addition of concentrated hydrochloric acid (0.1 mL, 1.2 mmol). The reaction mixture was stirred at room temperature for 2 h. After 2 h the reaction mixture was cooled in an ice bath and was neutralized with saturated sodium bicarbonate solution. The resulting mixture was purified by preparative HPLC (Phenominex Synergi 4 u Hydro-RR 80 Å 150×30 mm column, 15%-85% acetonitrile/water gradient in 30 min run) to afford the product. ¹H NMR (400 MHz, Methanol-d4) δ 7.79 (d, J=7.7 Hz, 1H), 7.37-7.17 (m, 3H), 7.16 (d, J=7.7 Hz, 2H), 6.84 (dd, J=4.5, 2.0 Hz, 1H), 6.73 (dd, J=4.5, 1.9 Hz, 1H), 5.49 (t, J=5.0 Hz, 1H), 4.66-4.57 (m, 1H), 4.54-4.29 (m, 3H), 3.95-3.70 (m, 3H), 1.65 (q, J=11.2, 10.5 Hz, 5H), 1.30-1.13 (m, 7H), 0.97-0.85 (m, 2H). ³¹P NMR (162 MHz, Methanol-d4) δ 3.25, 3.23. LCMS: MS m/z=615.19 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.135 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

Resolution of the Sp and Rp diastereomers. The product was purified via chiral preparatory HPLC (AD-H 5 um 21×250 mm, Heptane 70%, Ethanol 30%) to afford the diastereomers:

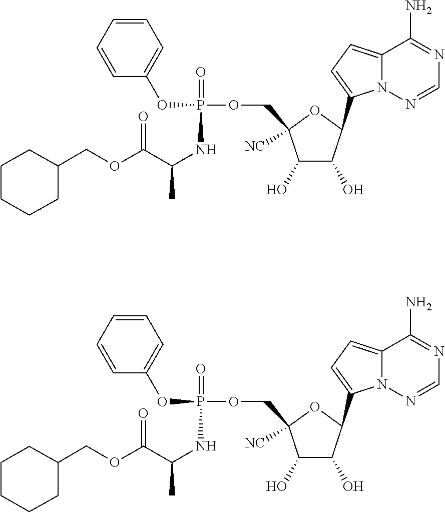 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCC1CCCCC1 ; C[C@H](N[P@](=O)(OC[C@@]1(C#N)O[C@@H](c2ccc3c(N)ncnn23)[C@H](O)[C@@H]1O)Oc1ccccc1)C(=O)OCC1CCCCC1]

### Example 247. First Eluting Diastereomer

¹H NMR (400 MHz, Methanol-d4) δ 7.78 (s, 1H), 7.38-7.22 (m, 2H), 7.20-7.09 (m, 3H), 6.91-6.79 (m, 1H), 6.73 (d, J=4.5 Hz, 1H), 5.50 (d, J=5.0 Hz, 1H), 4.62 (t, J=5.3 Hz, 1H), 4.54-4.39 (m, 2H), 4.35 (dd, J=10.9, 5.2 Hz, 1H), 4.00-3.76 (m, 3H), 1.68 (d, J=12.1 Hz, 5H), 1.41-1.03 (m, 7H), 0.93 (q, J=10.1, 8.6 Hz, 2H). ³¹P NMR (162 MHz, Methanol-d4) δ 3.25. LCMS: MS m/z=615.20 [M+1]; t_{R}=1.35 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.13 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 248. Second Eluting Diastereomer

¹H NMR (400 MHz, Methanol-d4) δ 7.80 (s, 1H), 7.33 (dd, J=8.5, 7.1 Hz, 2H), 7.26-7.11 (m, 3H), 6.84 (d, J=4.5 Hz, 1H), 6.73 (d, J=4.5 Hz, 1H), 5.49 (d, J=5.0 Hz, 1H), 4.61 (t, J=5.3 Hz, 1H), 4.49-4.24 (m, 3H), 4.02-3.58 (m, 3H), 1.79-1.44 (m, 5H), 1.33-1.04 (m, 7H), 0.89 (q, J=12.1 Hz, 2H). ³¹P NMR (162 MHz, Methanol-d4) δ 3.23. LCMS: MS m/z=615.20 [M+1]; t_{R}=1.36 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.144 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 249. methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(pyridin-3-yloxy)phosphoryl)-L-alaninate

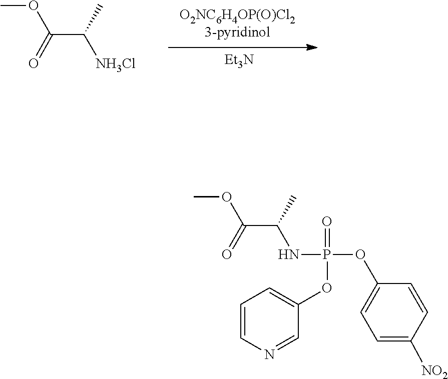

Methyl ((4-nitrophenoxy)(pyridin-3-yloxy)phosphoryl)-L-alaninate. 4-Nitrophenyl phosphorodichloridate (503 mg, 1.97 mmol) in dichloromethane (20 mL) was added dropwise over 10 minutes to a solution of L-alanine methyl ester hydrochloride (273 mg, 1.97 mmol) in dichloromethane (20 mL) at 0° C. After addition was complete, triethylamine (0.55 mL, 3.93 mmol) was added dropwise. After 90 minutes, 3-pyridinol (188 mg, 1.97 mmol) and triethylamine (0.28 mL, 1.97 mmol) were sequentially added at 0° C., and the resulting mixture was then allowed to warm to RT. After 30 minutes, the reaction mixture was washed with water (2×50 mL) and brine (50 mL), dried over anhydrous sodium sulfate, and concentrated under reduced pressure. The crude residue was purified by silica gel chromatography eluting with 20-100% ethyl acetate in hexanes to afford the product. ¹H NMR (400 MHz, Methanol-d₄) δ 8.53 (m, 1H), 8.43 (dq, J=4.8, 1.3 Hz, 1H), 8.39-8.07 (m, 2H), 7.79-7.32 (m, 4H), 4.17-3.95 (m, 1H), 3.72-3.57 (m, 3H), 1.41-1.26 (m, 3H). ³¹P NMR (162 MHz, Methanol-d₄) δ −0.86, −0.96. LCMS: MS m/z=382.15 [M+1], t_{R}=1.11 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

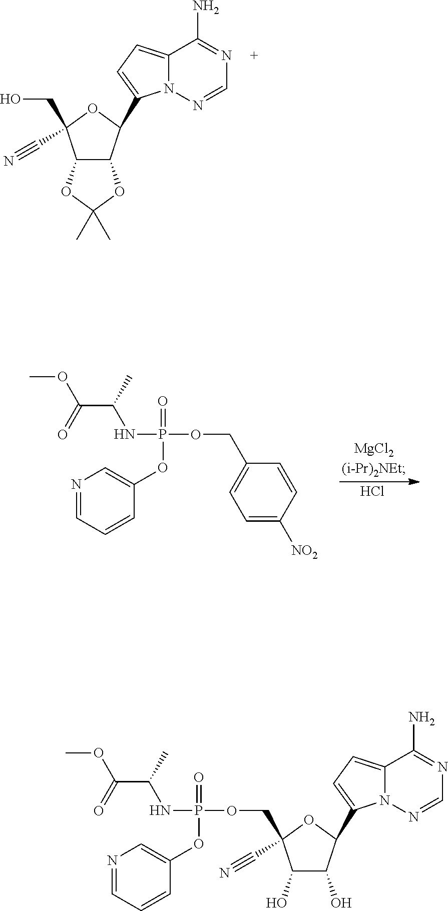 [SMILES: CC1(C)O[C@H]2[C@H](c3ccc4c(N)ncnn34)O[C@](C#N)(CO)[C@H]2O1 ; COC(=O)[C@H](C)NP(=O)(OCc1ccc([N+](=O)[O-])cc1)Oc1cccnc1 ; Cl ; COC(=O)[C@H](C)NP(=O)(OC[C@@]1(C#N)O[C@@H](c2ccc3c(N)ncnn23)[C@H](O)[C@@H]1O)Oc1cccnc1]

Methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(pyridin-3-yloxy)phosphoryl)-L-alaninate. N,N-diisopropylethylamine (0.13 mL, 0.76 mmol) and magnesium chloride (43 mg, 0.45 mmol) were added to a mixture of Intermediate 4 (100.0 mg, 0.30 mmol) and methyl ((4-nitrophenoxy)(pyridin-3-yloxy)phosphoryl)-L-alaninate (171 mg, 0.45 mmol) in tetrahydrofuran (7.5 mL) at RT. The mixture was heated to 55° C. After 2 h, the reaction mixture was allowed to cool to RT, diluted with ethyl acetate (30 mL) and the resulting mixture was washed with water (5×20 mL) and brine (20 mL). The organic layer was dried over anhydrous sodium sulfate and concentrated under reduced pressure. Concentrated aqueous hydrochloric acid solution (0.53 mL) was added dropwise to the crude residue in acetonitrile (7.5 mL) at 0° C. The mixture was warmed to RT. After 2 h, the reaction mixture was diluted with ethyl acetate (100 mL) and the resulting mixture was washed with saturated aqueous sodium carbonate solution (75 mL) and brine (75 mL). The organic layer was dried over anhydrous sodium sulfate and concentrated under reduced pressure. The crude residue was subjected to silica gel chromatography eluting with 0-20% methanol in dichloromethane to afford the product. ¹H NMR (400 MHz, Acetonitrile-d₃) δ 8.60 (s, 1H), 8.39 (d, J=2.7 Hz, 1H), 8.24 (d, J=5.5 Hz, 1H), 8.02-7.93 (m, 2H), 7.81 (dd, J=8.8, 5.6 Hz, 2H), 7.26 (d, J=4.6 Hz, 1H), 6.91 (d, J=4.8 Hz, 1H), 5.50 (d, J=4.7 Hz, 1H), 4.56-4.35 (m, 4H), 4.10 (q, J=7.3 Hz, 2H), 3.81 (s, 2H), 3.63 (s, 1H), 1.53-1.30 (m, 3H). ³¹P NMR (162 MHz, Acetonitrile-d₃) δ 3.90. LCMS: MS m/z=534.15 [M+1], t_{R}=0.81 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=2.82 min, HPLC system: Agilent 1100 series; Column: Gemini 5μ C18 110 A, 50×4.6 mm; Solvents: Acetonitrile with 0.1% TFA, Water with 0.1% TFA; Gradient: 0 min-9.0 min 2-95% ACN, 9.0 min-10.0 min 95% ACN at 2 m/min.

### Example 250. (2S,3R,4S,5R)-2-(4-aminopyrrolo[2,1-f][1,2,4]triazin-7-yl)-5-cyano-4-hydroxy-5-((((S)—(((S)-1-methoxy-1-oxopropan-2-yl)amino)(phenoxy)phosphoryl)oxy)methyl)tetrahydrofuran-3-yl isobutyrate

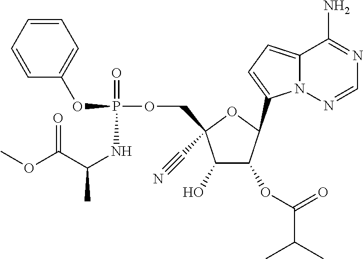 [SMILES: COC(=O)[C@H](C)N[P@](=O)(OC[C@@]1(C#N)O[C@@H](c2ccc3c(N)ncnn23)[C@H](OC(=O)C(C)C)[C@@H]1O)Oc1ccccc1]

Example 39. (54 mg, 0.1 mmol) was dissolved in 2 mL of anhydrous tetrahydrofuran. Isobutyric acid (37 μL, 0.4 mmol) and N,N′-diisopropylcarbodiimide (62 μL, 0.4 mmol) were added and the reaction mixture was stirred for 30 min. DMAP (12 mg, 0.1 mmol) was added and reaction mixture was stirred for 16 h. Methanol (0.5 mL) was added and the reaction mixture was stirred for 20 min. The reaction mixture was purified with prep HPLC without acid modifier (5-100% MeCN/water). Fractions containing the desired product were combined and extracted with ethyl acetate (15 mL). The organic extract was washed with brine (2×10 mL), dried over anhydrous sodium sulfate and concentrated under reduced pressure. The resulting material was dissolved in MeCN and water and freeze-dried to afford a mixture of mono- and bis-isobutyrate.

The mono-isobutyrate product was subjected to preparatory HPLC (Phenomenex Gemini 10 u C18 110 Å AXIA 250×21.2 mm column, 30-70% acetonitrile/water gradient with 0.1% TFA) to afford the product as a trifluoroacetate salt. ¹H NMR (400 MHz, CD₃CN) δ 7.93 (s, 1H), 7.41-7.32 (m, 2H), 7.25-7.12 (m, 4H), 6.89 (d, J=4.7 Hz, 1H), 5.64 (d, J=3.8 Hz, 1H), 5.57 (dd, J=5.8, 3.8 Hz, 1H), 4.76 (d, J=5.8 Hz, 1H), 4.49-4.26 (m, 3H), 3.61 (s, 3H), 2.68 (p, J=7.0 Hz, 1H), 1.30 (dd, J=7.1, 1.0 Hz, 3H), 1.20 (dd, J=8.3, 7.0 Hz, 6H). ³¹P NMR (162 MHz, CD₃CN) δ 2.72 ppm. LCMS: MS m/z=603.21 [M+1], t_{R}=1.22 min; LC system: Thermo Accela 1250 UHPLC; MS system: Thermo LCQ Fleet; Column: Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=4.61; HPLC system: Agilent 1100 series; Column: Phenomenex Kinetex C18, 2.6μ C18 110 A, 100×4.6 mm; Solvents: Acetonitrile with 0.1% TFA, Water with 0.1% TFA; Gradient: 0 min-8.5.0 min 2-98% ACN, 8.5 min-10.0 min 98% ACN at 1.5 mL/min.

### Example 251. 3,3-dimethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

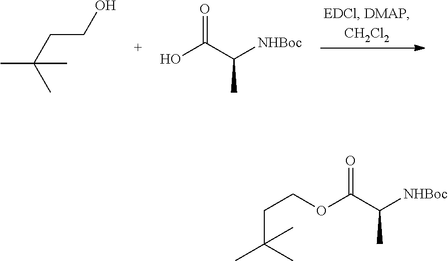

3,3-Dimethylbutyl (tert-butoxycarbonyl)-L-alaninate. Took up (tert-butoxycarbonyl)-L-alanine (22.38 g, 0.118 mol) in acetonitrile (100 mL) and 3,3-dimethylbutan-1-ol (10.07 g, 0.099 mol) followed by EDCI (19.89 g, 0.128 mol) and DMAP (18.06 g, 0.148 mol) in one portion. Allowed to stir at room temperature for 4 h. Diluted with dichloromethane and water. The layers were split and the organic layer was dried over anhydrous sodium sulfate, filtered and was concentrated under reduced pressure. Purified by silica gel chromatography 0-25% ethyl acetate/hexane to afford the compound. ¹H NMR (400 MHz, DMSO-d6) δ 7.22 (d, J=7.4 Hz, 1H), 4.13-3.82 (m, 3H), 1.47 (t, J=7.2 Hz, 2H), 1.35 (s, 9H), 1.19 (d, J=7.3 Hz, 3H), 0.88 (s, 9H).

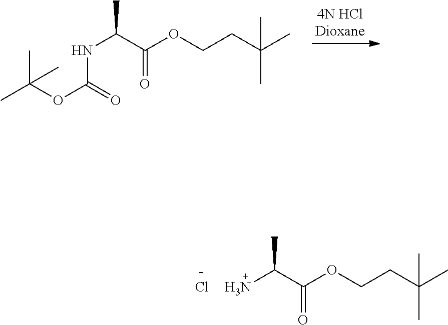 [SMILES: C[C@H](NC(=O)OC(C)(C)C)C(=O)OCCC(C)(C)C ; Cl ; C[C@H](N)C(=O)OCCC(C)(C)C]

(S)-1-(3,3-dimethylbutoxy)-1-oxopropan-2-aminium chloride. Took up 3,3-dimethylbutyl (tert-butoxycarbonyl)-L-alaninate (20.9 g, 0.076 mol) in anhydrous dichloromethane (200 mL) and 4 N HCl in dioxane (95.57 mL, 0.382 mol). Stirred at ambient temperature for 4 h. Concentrated under reduced pressure and co-evaporated with dichloromethane. Placed under high vacuum overnight and the compound was used as is without purification for the next step. ¹H NMR (400 MHz, DMSO-d6) δ 8.67 (s, 3H), 4.32-4.07 (m, 2H), 3.97 (d, J=7.2 Hz, 1H), 1.52 (t, J=7.3 Hz, 2H), 1.39 (d, J=7.1 Hz, 3H), 0.89 (s, 9H).

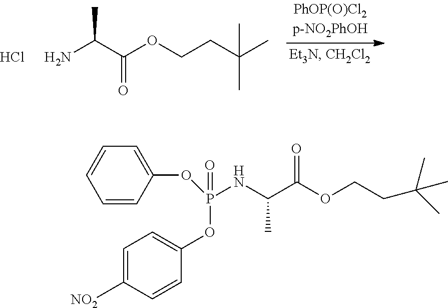 [SMILES: Cl ; C[C@H](N)C(=O)OCCC(C)(C)C ; C[C@H](NP(=O)(Oc1ccccc1)Oc1ccc([N+](=O)[O-])cc1)C(=O)OCCC(C)(C)C]

3,3-Dimethylbutyl ((4-nitrophenoxy)(phenoxy)phosphoryl)-L-alaninate. To a solution of (S)-1-(3,3-dimethylbutoxy)-1-oxopropan-2-aminium chloride (15.93 g, 75.96 mmol) and phenyl dichlorophosphate (11.3 mL, 75.96 mmol) in anhydrous dichloromethane (300 mL) was added triethylamine (23.5 mL, 167.1 mmol) at 0° C. under argon atmosphere. The resulting mixture was stirred for 1 h at 0° C. 4-Nitrophenol (10.57 g, 75.96 mmol) and triethylamine (11.74 mL, 83.56 mmol) were then added. After 1 h stirring at 0° C., the reaction mixture was diluted with Et₂O and the solids were filtered off. The crude product was concentrated under reduced pressure and was purified by silica gel chromatography (80 g SiO₂ Combiflash HP Gold Column, 100% dichloromethane followed by 0-35% ethyl acetate/hexanes) to afford the product. ¹H NMR (400 MHz, DMSO-d6) δ 8.28 (d, J=9.1 Hz, 2H), 7.53-7.34 (m, 4H), 7.30-7.16 (m, 3H), 6.66 (td, J=13.2, 10.0 Hz, 1H), 4.06-3.88 (m, 3H), 1.40-1.29 (m, 2H), 1.24-1.11 (m, 3H), 0.83 (s, 9H). ³¹P NMR (162 MHz, DMSO-d6) δ −1.26, −1.57. LCMS: MS m/z=450.96 [M+1]; t_{R}=1.71 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

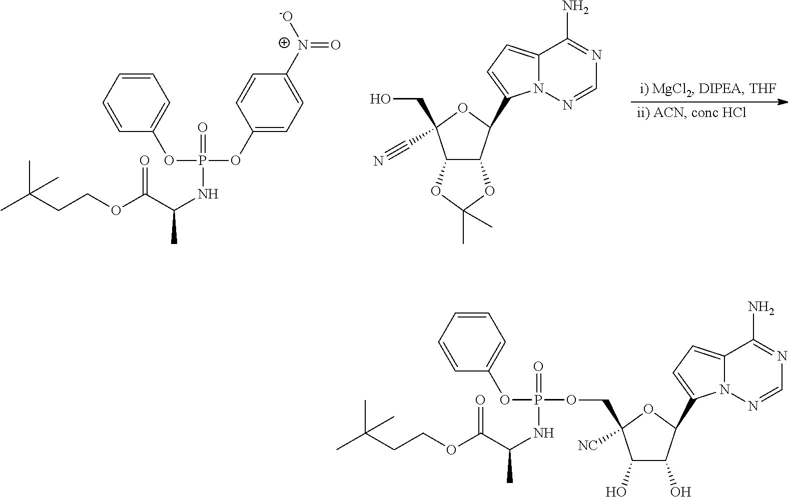

3,3-dimethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. To a mixture of Intermediate 4 (0.05 g, 0.151 mmol), intermediate 3,3-dimethylbutyl ((4-nitrophenoxy)(phenoxy)phosphoryl)-L-alaninate (0.078 g, 0.174 mmol), and magnesium chloride (0.029 g, 0.302 mmol) was added tetrahydrofuran (0.75 mL) at room temperature followed by the addition of N,N-Diisopropylethylamine (0.066 mL, 0.377 mmol). The resulting mixture was stirred at 50° C. for 1.5 h. The reaction mixture was then concentrated under reduced pressure and the residue obtained was diluted with saturated sodium chloride solution and ethyl acetate. The layers were split and the organic layer was dried over anhydrous sodium sulfate, filtered and was concentrated under reduced pressure. The crude residue was purified via SiO₂ column chromatography (40 g SiO₂ Combiflash HP Gold Column, 100% Dichloromethane-14% Methanol in dichloromethane). Pure fractions obtained were collected and concentrated under reduced pressure. The residue obtained was dissolved in an anhydrous acetonitrile (2 mL) and was cooled in an ice bath followed by the dropwise addition of concentrated hydrochloric acid (0.3 mL, 3.6 mmol). The reaction mixture was stirred at room temperature for 1 h. After 1 h the reaction mixture was cooled in an ice bath and was neutralized with 3 N aqueous sodium hydroxide solution. The resulting mixture was purified by preparative HPLC (Phenominex Synergi 4 u Hydro-RR 80 Å 150×30 mm column, 15%-85% acetonitrile/water gradient in 30 min run) to afford the product. ¹H NMR (400 MHz, Methanol-d4) δ 7.79 (d, J=7.2 Hz, 1H), 7.37-7.12 (m, 5H), 6.84 (dd, J=4.5, 2.8 Hz, 1H), 6.73 (dd, J=4.5, 2.0 Hz, 1H), 5.50 (t, J=4.7 Hz, 1H), 4.62 (q, J=5.6 Hz, 1H), 4.54-4.29 (m, 3H), 4.11 (s, 1H), 4.15-3.99 (m, 1H), 3.93-3.81 (m, 1H), 1.49 (dt, J=12.0, 7.5 Hz, 2H), 1.29-1.21 (m, 3H), 0.89 (d, J=7.9 Hz, 9H). ³¹P NMR (162 MHz, Methanol-d₄) δ 3.25. LCMS: MS m/z=603.08 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 P L/min. HPLC: t_{R}=5.02 min (minor isomer), 5.039 min (major isomer); HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

Resolution of the Sp and Rp diastereomers. Example 251 was purified via chiral preparatory HPLC (AD-H 5 um 21×250 mm, Heptane 70%, Isopropanol 30%) to afford the diastereomers:

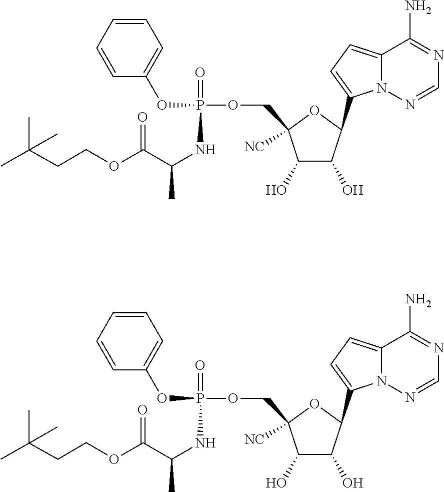 [SMILES: C[C@H](N[P@@](=O)(OC[C@@]1(C#N)O[C@@H](c2ccc3c(N)ncnn23)[C@H](O)[C@@H]1O)Oc1ccccc1)C(=O)OCCC(C)(C)C ; C[C@H](N[P@](=O)(OC[C@@]1(C#N)O[C@@H](c2ccc3c(N)ncnn23)[C@H](O)[C@@H]1O)Oc1ccccc1)C(=O)OCCC(C)(C)C]

### Example 252. First Eluting Diastereomer

¹H NMR (400 MHz, Methanol-d4) δ 7.78 (s, 1H), 7.33-7.24 (m, 2H), 7.19-7.11 (m, 3H), 6.84 (d, J=4.5 Hz, 1H), 6.73 (d, J=4.5 Hz, 1H), 5.50 (d, J=5.0 Hz, 1H), 4.62 (t, J=5.3 Hz, 1H), 4.55-4.43 (m, 2H), 4.35 (dd, J=10.9, 5.2 Hz, 1H), 4.16-4.06 (m, 2H), 3.92-3.79 (m, 1H), 1.55-1.46 (m, 2H), 1.34-1.22 (m, 3H), 0.90 (s, 9H). ³¹P NMR (162 MHz, Methanol-d4) δ 3.24. LCMS: MS m/z=603.06 [M+1]; t_{R}=1.20 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.024 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 253. Second Eluting Diastereomer

¹H NMR (400 MHz, Methanol-d4) δ 7.79 (s, 1H), 7.33 (dd, J=8.4, 7.3 Hz, 2H), 7.27-7.13 (m, 3H), 6.84 (d, J=4.5 Hz, 1H), 6.73 (d, J=4.5 Hz, 1H), 5.49 (d, J=5.1 Hz, 1H), 4.61 (t, J=5.3 Hz, 1H), 4.48-4.30 (m, 3H), 4.15-3.97 (m, 2H), 3.88 (dd, J=9.9, 7.1 Hz, 1H), 1.47 (t, J=7.4 Hz, 2H), 1.26 (dd, J=7.2, 1.0 Hz, 3H), 0.88 (s, 9H). ³¹P NMR (162 MHz, Methanol-d4) δ 3.25 LCMS: MS m/z=603.09 [M+1]; t_{R}=1.22 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.045 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 254. 2,2-dimethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

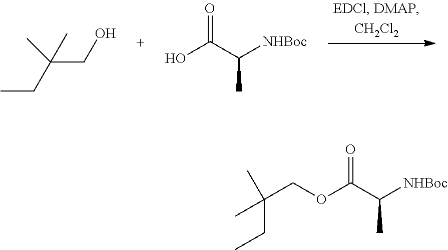

2,2-Dimethylbutyl (tert-butoxycarbonyl)-L-alaninate. Took up (tert-butoxycarbonyl)-L-alanine (11.11 g, 0.059 mol) in acetonitrile (60 mL) and 2,2-dimethylbutan-1-ol (5.0 g, 0.049 mol) followed by EDCI (9.876 g, 0.064 mol) and DMAP (8.967 g, 0.073 mol) in one portion. Allowed to stir at room temperature for 4 h. Diluted with dichloromethane and water. The layers were split and the organic layer was dried over anhydrous sodium sulfate, filtered and was concentrated under reduced pressure. Purification by silica gel chromatography 0-20% ethyl acetate/hexane to afford the compound. ¹H NMR (400 MHz, DMSO-d6) δ 7.26 (d, J=7.5 Hz, 1H), 3.99 (p, J=7.4 Hz, 1H), 3.81 (d, J=10.6 Hz, 1H), 3.66 (d, J=10.6 Hz, 1H), 1.35 (s, 9H), 1.29 (d, J=18.5 Hz, 3H), 0.84-0.72 (m, 11H).

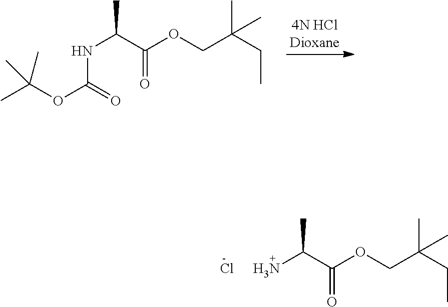 [SMILES: CCC(C)(C)COC(=O)[C@H](C)NC(=O)OC(C)(C)C ; Cl ; CCC(C)(C)COC(=O)[C@H](C)N]

(S)-1-(2,2-dimethylbutoxy)-1-oxopropan-2-aminium chloride. Took up 2,2-dimethylbutyl (tert-butoxycarbonyl)-L-alaninate (10.34 g, 0.038 mol) in anhydrous dichloromethane (100 mL) and 4 N HCl in dioxane (47.28 mL, 0.189 mol). Stirred at ambient temperature for 4 h. Concentrated under reduced pressure and co-evaporated with dichloromethane. Placed under high vacuum for overnight and the compound was used as is without purification for the next step. ¹H NMR (400 MHz, DMSO-d6) δ 8.65 (s, 3H), 4.05 (q, J=7.2 Hz, 1H), 3.91 (d, J=10.6 Hz, 1H), 3.79 (d, J=10.6 Hz, 1H), 1.42 (d, J=7.2 Hz, 3H), 1.26 (q, J=7.6 Hz, 2H), 0.87-0.73 (m, 9H).

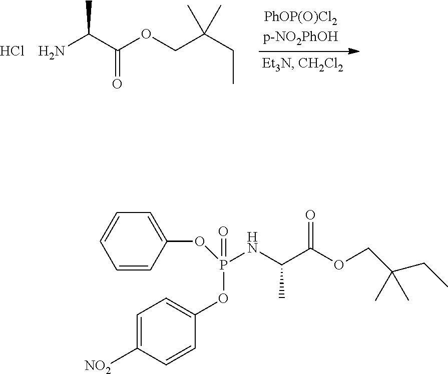 [SMILES: Cl ; CCC(C)(C)COC(=O)[C@H](C)N ; CCC(C)(C)COC(=O)[C@H](C)NP(=O)(Oc1ccccc1)Oc1ccc([N+](=O)[O-])cc1]

2,2-dimethylbutyl ((4-nitrophenoxy)(phenoxy)phosphoryl)-L-alaninate. To a solution of (S)-1-(2,2-dimethylbutoxy)-1-oxopropan-2-aminium chloride (7.9 g, 37.67 mmol) and phenyl dichlorophosphate (5.605 mL, 37.67 mmol) in anhydrous dichloromethane (150 mL) was added triethylamine (11.64 mL, 82.87 mmol) at 0° C. under argon atmosphere. The resulting mixture was stirred for 1 h at 0° C. 4-Nitrophenol (5.24 g, 37.67 mmol) and triethylamine (5.82 mL, 41.43 mmol) were then added. After 1 h stirring at 0° C., the reaction mixture was diluted with Et₂O and the solids were filtered off. The crude product was concentrated under reduced pressure and was purified by silica gel chromatography (80 g SiO₂ Combiflash HP Gold Column, 100% dichloromethane followed by 0-35% ethyl acetate/hexanes) to afford the compound. ¹H NMR (400 MHz, Methanol-d4) δ 8.32-8.23 (m, 2H), 7.52-7.34 (m, 4H), 7.31-7.18 (m, 3H), 4.15-4.02 (m, 1H), 3.86-3.74 (m, 2H), 1.39-1.28 (m, 3H), 1.32-1.19 (m, 2H), 0.89-0.76 (m, 9H). ³¹P NMR (162 MHz, Methanol-d4) δ −1.35, −1.57 LCMS: MS m/z=450.94 [M+1]; t_{R}=1.71 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min.

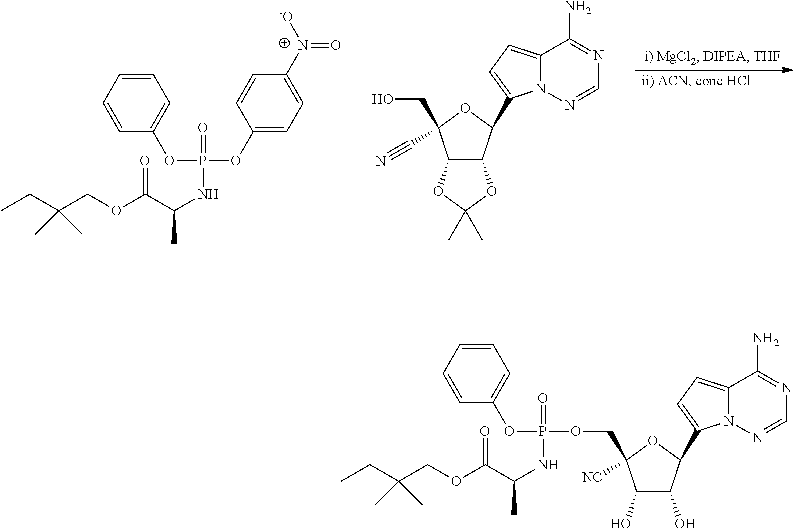

2,2-dimethylbutyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. To a mixture of Intermediate 4 (0.05 g, 0.151 mmol), 2,2-dimethylbutyl ((4-nitrophenoxy)(phenoxy)phosphoryl)-L-alaninate (0.078 g, 0.174 mmol), and magnesium chloride (0.029 g, 0.302 mmol) was added tetrahydrofuran (0.75 mL) at room temperature followed by the addition of N,N-Diisopropylethylamine (0.066 mL, 0.377 mmol). The resulting mixture was stirred at 50° C. for 1.5 h. The reaction mixture was then concentrated under reduced pressure and the residue obtained was diluted with saturated sodium chloride solution and ethyl acetate. The layers were split and the organic layer was dried over anhydrous sodium sulfate, filtered and was concentrated under reduced pressure. The residue obtained was dissolved in an anhydrous acetonitrile (1 mL) and was cooled in an ice bath followed by the dropwise addition of concentrated hydrochloric acid (0.1 mL, 1.2 mmol). The reaction mixture was stirred at room temperature for 1 h. After 1 h the reaction mixture was cooled in an ice bath and was neutralized with saturated aqueous sodium bicarbonate solution. The resulting mixture was purified by preparative HPLC (Phenominex Synergi 4 u Hydro-RR 80 Å 150×30 mm column, 15%-85% acetonitrile/water gradient in 30 min run) to afford the product. ¹H NMR (400 MHz, Methanol-d4) δ 7.79 (d, J=6.9 Hz, 1H), 7.40-7.08 (m, 5H), 6.87-6.80 (m, 1H), 6.75-6.70 (m, 1H), 5.49 (d, J=5.0 Hz, 1H), 4.61 (dt, J=7.7, 5.4 Hz, 1H), 4.54-4.26 (m, 3H), 3.98-3.73 (m, 2H), 3.66 (d, J=10.7 Hz, 1H), 1.33-1.17 (m, 5H), 0.92-0.72 (m, 9H). ³¹P NMR (162 MHz, Methanol-d₄) δ 3.26. LCMS: MS m/z=603.10 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.026 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

Resolution of the Sp and Rp diastereomers. Example 254 was purified via chiral preparatory HPLC (IE SFC 5 um 21×250 mm, Heptane 70%, Ethanol 30%) to afford the diastereomers:

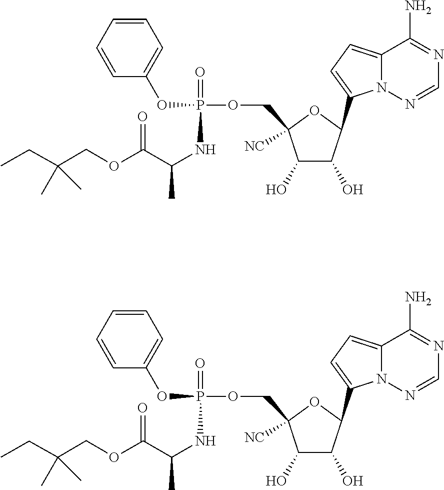 [SMILES: CCC(C)(C)COC(=O)[C@H](C)N[P@@](=O)(OC[C@@]1(C#N)O[C@@H](c2ccc3c(N)ncnn23)[C@H](O)[C@@H]1O)Oc1ccccc1 ; CCC(C)(C)COC(=O)[C@H](C)N[P@](=O)(OC[C@@]1(C#N)O[C@@H](c2ccc3c(N)ncnn23)[C@H](O)[C@@H]1O)Oc1ccccc1]

### Example 255. First Eluting Diastereomer

¹H NMR (400 MHz, Methanol-d4) δ 7.80 (s, 1H), 7.37-7.28 (m, 2H), 7.27-7.13 (m, 3H), 6.84 (d, J=4.5 Hz, 1H), 6.73 (d, J=4.6 Hz, 1H), 5.49 (d, J=5.0 Hz, 1H), 4.60 (t, J=5.3 Hz, 1H), 4.49-4.29 (m, 3H), 3.93 (dd, J=10.0, 7.1 Hz, 1H), 3.80 (d, J=10.7 Hz, 1H), 3.66 (d, J=10.7 Hz, 1H), 1.32-1.20 (m, 5H), 0.82 (s, 6H), 0.85-0.74 (m, 3H). ³¹P NMR (162 MHz, Methanol-d4) δ 3.27 LCMS: MS m/z=603.06 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.013 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 256. Second Eluting Diastereomer

¹H NMR (400 MHz, Methanol-d4) δ 7.78 (d, J=1.4 Hz, 1H), 7.28 (dd, J=8.6, 7.2 Hz, 2H), 7.19-7.10 (m, 3H), 6.85 (d, J=4.4 Hz, 1H), 6.73 (d, J=4.6 Hz, 1H), 5.50 (d, J=5.1 Hz, 1H), 4.62 (t, J=5.3 Hz, 1H), 4.48 (dd, J=15.3, 5.6 Hz, 2H), 4.35 (dd, J=10.9, 5.2 Hz, 1H), 3.99-3.87 (m, 1H), 3.84 (d, J=10.7 Hz, 1H), 3.77 (d, J=10.7 Hz, 1H), 1.34-1.23 (m, 5H), 0.89-0.77 (m, 9H). ³¹P NMR (162 MHz, Methanol-d4) δ 3.26. LCMS: MS m/z=603.06 [M+1]; t_{R}=1.21 min; LC system: Thermo Accela 1250 UHPLC; MS system: Thermo LCQ Fleet; Column: Phenomenex Kinetex 2.6μ XB-C18 100 A, 50×3.0 mm; Solvents: acetonitrile with 0.1% formic acid, water with 0.1% formic acid; Gradient: 0 min-1.8 min 2-100% acetonitrile, 1.8 min-1.85 min 100%-2% acetonitrile, 1.85 min-2.00 min 2% ACN at 1800 μL/min. HPLC: t_{R}=5.007 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 257. (1-methylcyclopropyl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

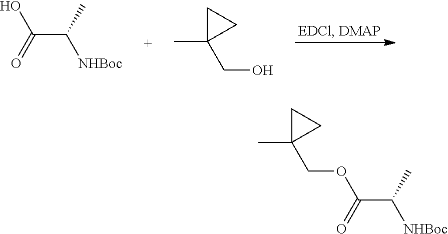

(1-Methylcyclopropyl)methyl (tert-butoxycarbonyl)-L-alaninate. 1-(3-Dimethylaminopropyl)-3-ethylcarbodiimide hydrochloride (1.22 g, 6.36 mmol) was added to a solution of (tert-butoxycarbonyl)-L-analine (1.00 g, 5.29 mmol) in 10 mL of acetonitrile under an atmosphere of argon. After 15 minutes, 4-(dimethylamino)-pyridine (0.71 g, 5.81 mmol) then (1-methylcyclopropyl)methanol (0.51 mL, 5.29 mmol) was added. The reaction was stirred for 2 h at room temperature. The reaction was diluted with ethyl acetate and washed with a 5% citric acid solution in water (2×10 mL). The organics were washed with saturated aqueous sodium bicarbonate (10 mL), water (5 mL) then brine (10 mL). The organics were dried over sodium sulfate, filtered and were concentrated under reduced pressure. The residue was purified by silica gel chromatography (0-10% ethyl acetate in hexanes) to afford the product. ¹H NMR (400 MHz, chloroform-d) δ 5.06 (s, 1H), 4.49-4.28 (m, 1H), 4.05-3.83 (m, 2H), 1.44 (s, 9H), 1.40 (d, J=7.2 Hz, 3H), 1.12 (s, 3H), 0.53-0.43 (m, 2H), 0.41-0.32 (m, 2H).

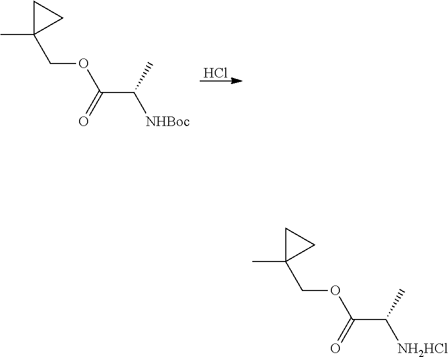

(1-Methylcyclopropyl)methyl L-alaninate hydrochloride. Hydrogen chloride (4 M in 1,4-dioxane, 3.7 mL, 14.80 mmol) was added to a solution of (1-methylcyclopropyl)methyl (tert-butoxycarbonyl)-L-alaninate (1.85 g, 7.19 mmol) in dichloromethane (10 mL) at 0° C. After 1 h, the reaction was concentrated. The residue was taken up in dichloromethane (10 mL) and concentrated (2×). The product was taken on to the next reaction without further purification. ¹H NMR (400 MHz, chloroform-d) δ 8.81 (s, 3H), 4.31-4.17 (m, 1H), 4.06 (d, J=11.0 Hz, 1H), 3.94 (d, J=11.0 Hz, 1H), 1.75 (d, J=7.1 Hz, 3H), 1.13 (s, 3H), 0.53-0.46 (m, 2H), 0.45-0.36 (m, 2H).

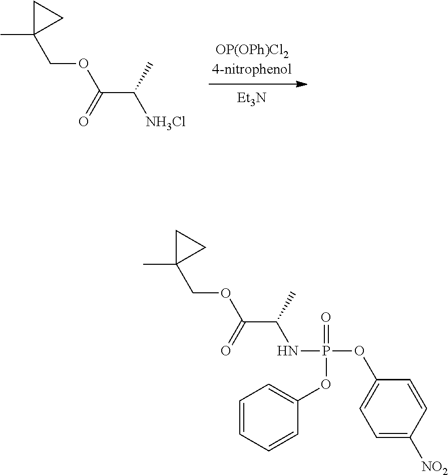

(1-Methylcyclopropyl)methyl ((4-nitrophenoxy)(phenoxy)phosphoryl)-L-alaninate. Phenyl dichlorophosphate (0.82 mL, 5.45 mmol) and triethylamine (1.58 mL, 11.36 mmol) were sequentially added to a suspension of (1-methylcyclopropyl)methyl L-alaninate hydrochloride (880 mg, 4.54 mmol) in dichloromethane (15 mL) at 0° C. After 1 h, 4-nitrophenol (0.63 g, 4.54 mmol) and triethylamine (0.79 mL, 5.8 mmol) were then sequentially added at 0° C., and the resulting mixture was then allowed to warm to room temperature. After 2.5 h, the reaction mixture was diluted with dichloromethane (50 mL), washed with saturated aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, dimethylsulfoxide-d₆) δ 8.34-8.27 (m, 2H), 7.55-7.37 (m, 4H), 7.31-7.17 (m, 3H), 6.78-6.65 (m, 1H), 4.13-3.97 (m, 1H), 3.89-3.76 (m, 2H), 1.31-1.21 (m, 3H), 1.02 (d, J=1.5 Hz, 3H), 0.49-0.37 (m, 2H), 0.35-0.23 (m, 2H). ³¹P NMR (162 MHz, dimethylsulfoxide-d₆) δ −1.25, −1.44. LCMS: MS m/z=433.02 [M−1], t_{R}=1.75 min (minor), 1.77 (major); LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

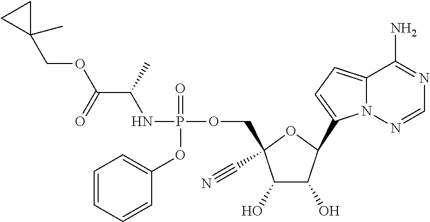 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC1(C)CC1]

(1-methylcyclopropyl)methyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Tetrahydrofuran (0.5 mL) was added to a mixture of Intermediate 4 (40 mg, 0.12 mmol), (1-methylcyclopropyl)methyl ((4-nitrophenoxy)(phenoxy)phosphoryl)-L-alaninate (68.17 mg, 0.16 mmol), and magnesium chloride (17.24 mg, 0.18 mmol) at RT. The mixture was stirred at RT for 20 min. N,N-Diisopropylethylamine (0.053 mL, 0.30 mmol) was added. The reaction was heated to 50° C. for 2 h, the reaction mixture was allowed to cool to RT, and diluted with ethyl acetate (2 mL). The organics were washed with water (2 mL), dried over sodium sulfate, filtered and were concentrated under reduced pressure. Aqueous hydrochloric acid solution (0.1 mL) was added dropwise to a solution of the residue in acetonitrile (2 mL) at 0° C. After 1 h, the reaction mixture was diluted with ethyl acetate (5 mL) and the resulting mixture was washed with saturated aqueous sodium carbonate solution (5 mL) and brine (5 mL). The organic layer was dried over anhydrous sodium sulfate and concentrated under reduced pressure. The residue was purified by HPLC chromatography (Phenomenex Gemini 5 μm C18 110 Å, 100×30 mm, 5-100% acetonitrile in water) to afford the product. LCMS: MS m/z=586.93 [M+1], t_{R}=1.39 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min

Resolution of the Sp and Rp diastereomers. The product was purified via chiral preparatory HPLC (SFC AD-H 5 μm, 250×21 mm, water 70%, isopropanol 30%) to afford the diastereomers:

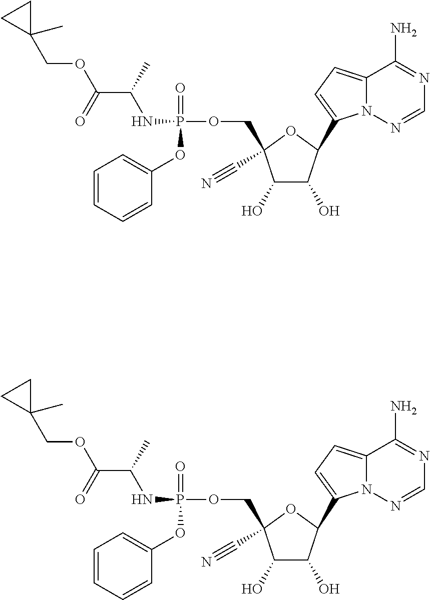 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC1(C)CC1 ; C[C@H](N[P@@](=O)(OC[C@@]1(C#N)O[C@@H](c2ccc3c(N)ncnn23)[C@H](O)[C@@H]1O)Oc1ccccc1)C(=O)OCC1(C)CC1]

### Example 258. First Eluting Diastereomer

¹H NMR (400 MHz, methanol-d4) δ 7.95 (s, 1H), 7.35-7.24 (m, 3H), 7.21-7.13 (m, 3H), 6.91 (d, J=4.7 Hz, 1H), 5.54 (d, J=5.2 Hz, 1H), 4.61-4.54 (m, 1H), 4.51-4.44 (m, 2H), 4.42-4.36 (m, 1H), 4.01-3.85 (m, 3H), 1.37-1.30 (m, 3H), 1.10 (s, 3H), 0.52-0.45 (m, 2H), 0.38-0.32 (m, 2H). ³¹P NMR (162 MHz, methanol-d₄) δ 3.32. LCMS: MS m/z=586.93 [M+1], t_{R}=1.37 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.75 min; Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 259. Second Eluting Diastereomer

¹H NMR (400 MHz, methanol-d4) δ 8.01 (s, 1H), 7.38-7.28 (m, 3H), 7.27-7.16 (m, 3H), 6.93 (d, J=4.7 Hz, 1H), 5.53 (d, J=5.2 Hz, 1H), 4.56-4.52 (m, 1H), 4.45-4.34 (m, 3H), 4.00-3.88 (m, 2H), 3.79 (d, J=11.1 Hz, 1H), 1.37-1.30 (m, 3H), 1.08 (s, 3H), 0.45 (t, J=2.8 Hz, 2H), 0.36-0.27 (m, 2H). ³¹P NMR (162 MHz, methanol-d₄) δ 3.27. LCMS: MS m/z=586.93 [M+1], t_{R}=1.39 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.77 min; Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 260. 3,3,3-trifluoro-2,2-dimethylpropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate

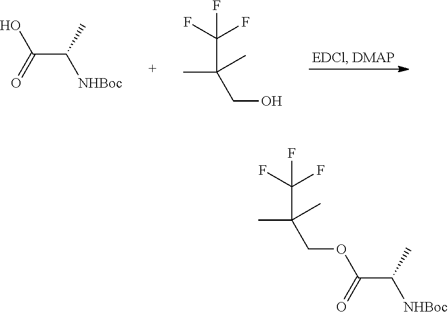

3,3,3-Trifluoro-2,2-dimethylpropyl (tert-butoxycarbonyl)-L-alaninate. 1-(3-Dimethylaminopropyl)-3-ethylcarbodiimide hydrochloride (2.43 g, 12.69 mmol) was added to a solution of (tert-butoxycarbonyl)-L-alanine (2.00 g, 10.57 mmol) in 10 mL of acetonitrile under an atmosphere of argon. After 15 min, 4-(dimethylamino)-pyridine (1.42 g, 11.62 mmol) then 3,3,3-trifluoro-2,2-dimethylpropan-1-ol (1.69 mL, 10.55 mmol) was added. The reaction was stirred for 2 h at RT. The reaction was diluted with ethyl acetate and washed with a 5% citric acid solution in water (2×10 mL). The organics were washed with saturated aqueous sodium bicarbonate (10 mL), water (5 mL) then brine (10 mL). The organics were dried over sodium sulfate, filtered and were concentrated. The residue was purified by silica gel chromatography (0-10% ethyl acetate in hexanes) to afford the product. ¹H NMR (400 MHz, dimethylsulfoxide-d) δ 7.32 (d, J=7.3 Hz, 1H), 4.15 (d, J=11.6 Hz, 1H), 4.08-3.95 (m, 2H), 1.37 (s, 9H), 1.25 (d, J=7.3 Hz, 3H), 1.13-1.09 (m, 6H).

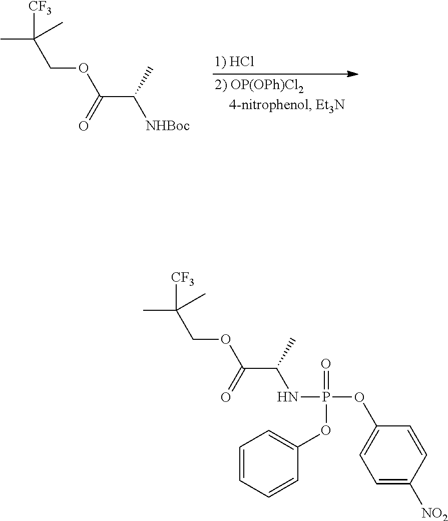 [SMILES: C[C@H](NC(=O)OC(C)(C)C)C(=O)OCC(C)(C)C(F)(F)F ; C[C@H](NP(=O)(Oc1ccccc1)Oc1ccc([N+](=O)[O-])cc1)C(=O)OCC(C)(C)C(F)(F)F]

3,3,3-Trifluoro-2,2-dimethylpropyl ((4-nitrophenoxy)(phenoxy)phosphoryl)-L-alaninate. 4 M hydrogen chloride in 1,4-dioxane (10 mL, 40.00 mmol) was added to a solution of 3,3,3-trifluoro-2,2-dimethylpropyl (tert-butoxycarbonyl)-L-alaninate (1.88 g, 6.00 mmol) in dichloromethane (5 mL). After 1 h, the reaction was concentrated. The residue was dissolved in dichloromethane (15 mL) and cooled to 0° C. Phenyl dichlorophosphate (1.07 mL, 7.21 mmol) and triethylamine (1.83 mL, 13.22 mmol) were added sequentially. After 1 h, 4-nitrophenol (0.836 g, 6.00 mmol) and triethylamine (0.92 mL, 7.00 mmol) were then sequentially added at 0° C., and the resulting mixture was then allowed to warm to RT. After 2.5 h, the reaction mixture was diluted with dichloromethane (50 mL), washed with saturated aqueous sodium bicarbonate solution (50 mL) and brine (50 mL), dried over anhydrous sodium sulfate, and concentrated under reduced pressure. The crude residue was purified by silica gel chromatography eluting with 0-100% ethyl acetate in hexanes to afford the product. ¹H NMR (400 MHz, dimethylsulfoxide-d₆) δ 8.30 (d, J=8.9 Hz, 2H), 7.56-7.36 (m, 4H), 7.35-7.16 (m, 3H), 6.85-6.64 (m, 1H), 4.18-3.94 (m, 3H), 1.30-1.23 (m, 3H), 1.10 (s, 6H). ³¹P NMR (162 MHz, dimethylsulfoxide-d₆) δ −1.29, −1.46. ¹⁹F NMR (376 MHz, dimethylsulfoxide-d₆) δ −76.19, −76.19.

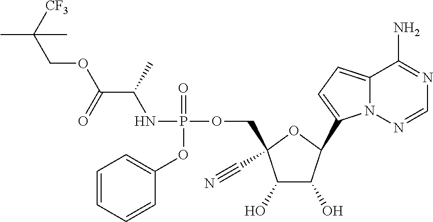 [SMILES: C[C@H](NP(=O)(OC[C@@]1(C#N)O[C@@H](c2ccc3c(N)ncnn23)[C@H](O)[C@@H]1O)Oc1ccccc1)C(=O)OCC(C)(C)C(F)(F)F]

3,3,3-Trifluoro-2,2-dimethylpropyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(phenoxy)phosphoryl)-L-alaninate. Tetrahydrofuran (0.5 mL) was added to a mixture of Intermediate 4 (59 mg, 0.18 mmol), 3,3,3-trifluoro-2,2-dimethylpropyl ((4-nitrophenoxy)(phenoxy)phosphoryl)-L-alaninate (113.52 mg, 0.23 mmol), and magnesium chloride (25.43 mg, 0.27 mmol) at room temperature. The mixture was stirred for 20 min. N,N-Diisopropylethylamine (77.54 μL, 0.44 mmol) was added. The reaction was heated to 50° C. for 2 h, the reaction mixture was allowed to cool to room temperature, and diluted with ethyl acetate (2 mL). The organics were washed with water (2 mL), dried over sodium sulfate, filtered and were concentrated. Aqueous hydrochloric acid solution (0.1 mL, 12 M) was added dropwise to a solution of the residue in acetonitrile (2 mL) at 0° C. After 1 h, the reaction mixture was diluted with ethyl acetate (5 mL) and the resulting mixture was washed with saturated aqueous sodium carbonate solution (5 mL) and brine (5 mL). The organic layer was dried over anhydrous sodium sulfate and concentrated under reduced pressure. The residue was purified by HPLC chromatography (Phenomenex Gemini 5 μm C18 110 Å, 100×30 mm, 5-100% acetonitrile in water) to afford the product. LCMS: MS m/z=642.97 [M+1], t_{R}=1.46 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

Resolution of the Sp and Rp diastereomers. The product was purified via chiral preparatory HPLC (SFC IA 5 μm, 250×21 mm, water 70%, isopropanol 30%) to afford the diastereomers:

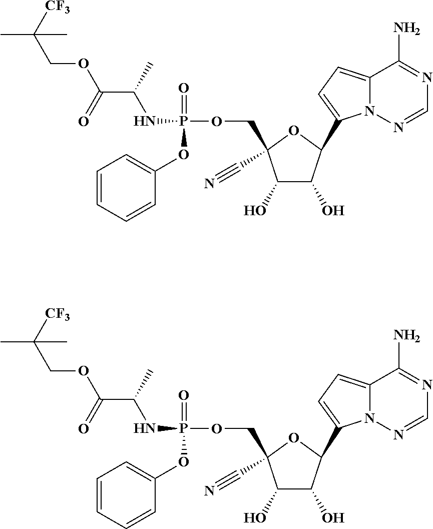 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OCC(C)(C)C(F)(F)F ; C[C@H](N[P@@](=O)(OC[C@@]1(C#N)O[C@@H](c2ccc3c(N)ncnn23)[C@H](O)[C@@H]1O)Oc1ccccc1)C(=O)OCC(C)(C)C(F)(F)F]

### Example 261. First Eluting Diastereomer

¹H NMR (400 MHz, methanol-d₄) δ 7.98 (s, 1H), 7.39-7.24 (m, 3H), 7.20-7.14 (m, 3H), 6.94 (d, J=4.7 Hz, 1H), 5.54 (d, J=5.4 Hz, 1H), 4.58-4.53 (m, 1H), 4.50-4.43 (m, 2H), 4.43-4.36 (m, 1H), 4.16 (d, J=11.5 Hz, 1H), 4.06 (d, J=11.5 Hz, 1H), 4.03-3.91 (m, 1H), 1.38-1.33 (m, 3H), 1.16 (s, 3H), 1.15 (s, 3H). ³¹P NMR (162 MHz, methanol-d₄) δ 3.24. ¹⁹F NMR (376 MHz, methanol-d₄) δ −78.75. LCMS: MS m/z=642.97 [M+1], t_{R}=1.45 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.92 min; Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### Example 262. Second Eluting Diastereomer

¹H NMR (400 MHz, methanol-d₄) δ 8.02 (s, 1H), 7.38-7.32 (m, 3H), 7.27-7.17 (m, 3H), 6.95 (d, J=4.7 Hz, 1H), 5.53 (d, J=5.2 Hz, 1H), 4.55-4.50 (m, 1H), 4.43-4.33 (m, 3H), 4.13 (d, J=11.5 Hz, 1H), 4.00-3.92 (m, 2H), 1.33 (d, J=7.2, 1.1 Hz, 3H), 1.13 (s, 3H), 1.11 (s, 3H). ³¹P NMR (162 MHz, methanol-d₄) δ 3.19. ¹⁹F NMR (376 MHz, methanol-d₄) δ −78.79. LCMS: MS m/z=642.97 [M+1], t_{R}=1.40 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.91 min; Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 263. Ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(4-(tert-butyl)phenoxy)phosphoryl)-L-alaninate

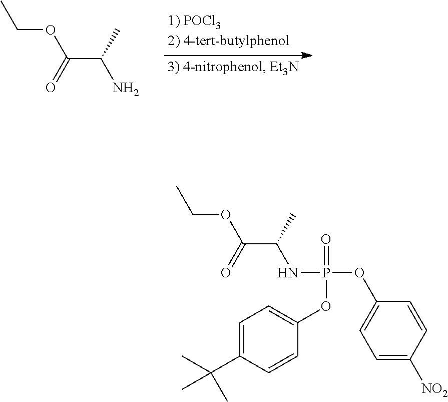

Ethyl ((4-(tert-butyl)phenoxy)(4-nitrophenoxy)phosphoryl)-L-alaninate. To a solution of phosphorus(V) oxychloride (0.61 mL, 6.52 mmol) in dichloromethane (20 mL) at −78° C. under an atmosphere of argon was added L-alanine ethyl ester (1.00 g, 6.52 mmol). Triethylamine (2.00 mL, 14.35 mmol) was added dropwise slowly. After 15 min, the reaction was allowed to warm to 0° C. After 30 minutes, the reaction was cooled to −78° C. and 4-tert-butylphenol (0.98 g, 6.52 mmol) was added. Triethylamine (0.91 mL, 6.52 mmol) was added. The reaction was allowed to warm to room temperature and stirred for 3 h. The reaction was cooled to 0° C. 4-nitrophenol (0.91 g, 6.52 mmol) was added followed by a dropwise addition of triethylamine (0.91 mL, 6.52 mmol). The reaction was allowed to warm to RT and stirred for 2 h. The reaction was diluted with ethyl acetate and washed with ammonium chloride, water, brine. The organics were dried over sodium sulfate, filtered and were concentrated. The product was isolated by silica gel chromatography (0-50% ethyl acetate in hexanes). ¹H NMR (400 MHz, dimethylsulfoxide-d₆) δ 8.31 (d, J=8.8 Hz, 2H), 7.57-7.36 (m, 4H), 7.24-7.09 (m, 2H), 6.76-6.57 (m, 1H), 4.12-3.77 (m, 3H), 1.30-1.21 (m, 12H), 1.11 (t, J=7.1 Hz, 3H). ³¹P NMR (162 MHz, dimethylsulfoxide-d₆) δ −1.16, −1.24. LCMS: MS m/z=451.00 [M+1], t_{R}=1.84 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

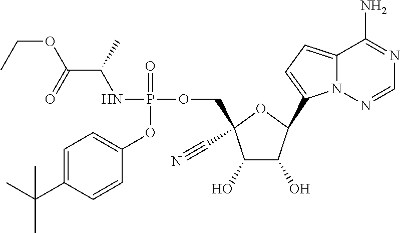 [SMILES: CCOC(=O)[C@H](C)NP(=O)(OC[C@@]1(C#N)O[C@@H](c2ccc3c(N)ncnn23)[C@H](O)[C@@H]1O)Oc1ccc(C(C)(C)C)cc1]

Ethyl ((((2R,3S,4R,5S)-5-(4-aminopyrrolo[2,1-f][1,2,4]triazin-7-yl)-2-cyano-3,4-dihydroxytetrahydrofuran-2-yl)methoxy)(4-(tert-butyl)phenoxy)phosphoryl)-L-alaninate. Tetrahydrofuran (0.5 mL) was added to a mixture of Intermediate 4 (40 mg, 0.12 mmol), ethyl ((4-(tert-butyl)phenoxy)(4-nitrophenoxy)phosphoryl)-L-alaninate (70.69 mg, 0.16 mmol), and magnesium chloride (17.24 mg, 0.18 mmol) at RT. The mixture was stirred at RT for 20 min. N,N-Diisopropylethylamine (52.57 μL, 0.30 mmol) was added. The reaction was heated to 50° C. for 2 h, the reaction mixture was allowed to cool to RT, and diluted with ethyl acetate (2 mL). The organics were washed with water (2 mL), dried over sodium sulfate, filtered and were concentrated under reduced pressure. Aqueous hydrochloric acid solution (0.1 mL, 12 M) was added dropwise to a solution of the residue in acetonitrile (2 mL) at 0° C. After 1 h, the reaction mixture was diluted with ethyl acetate (5 mL) and the resulting mixture was washed with saturated aqueous sodium carbonate solution (5 mL) and brine (5 mL). The organic layer was dried over anhydrous sodium sulfate and concentrated under reduced pressure. The residue was purified by HPLC chromatography (Phenomenex Gemini 5 μm C18 110 Å, 100×30 mm, 5-100% acetonitrile in water) to afford the product. LCMS: MS m/z=602.97 [M+1], t_{R}=1.44 min (minor), 1.46 min (major); LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min.

Resolution of the Sp and Rp diastereomers. The product was purified via chiral preparatory HPLC (SFC AD-H 5 μm, 250×21 mm, water 70%, isopropanol 30%) to afford the diastereomers:

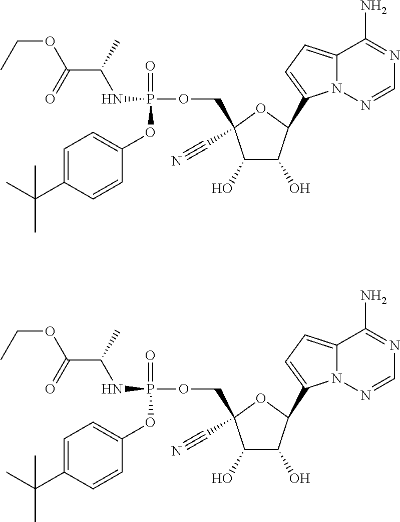 [SMILES: CCOC(=O)[C@H](C)N[P@](=O)(OC[C@@]1(C#N)O[C@@H](c2ccc3c(N)ncnn23)[C@H](O)[C@@H]1O)Oc1ccc(C(C)(C)C)cc1 ; CCOC(=O)[C@H](C)N[P@@](=O)(OC[C@@]1(C#N)O[C@@H](c2ccc3c(N)ncnn23)[C@H](O)[C@@H]1O)Oc1ccc(C(C)(C)C)cc1]

### Example 264. First Eluting Diastereomer

¹H NMR (400 MHz, methanol-d₄) δ 7.95 (s, 1H), 7.39-7.36 (m, 2H), 7.19-7.10 (m, 3H), 6.89 (d, J=4.7 Hz, 1H), 5.53 (d, J=5.4 Hz, 1H), 4.57 (t, J=5.5 Hz, 1H), 4.46-4.32 (m, 3H), 4.12-4.01 (m, 2H), 3.93-3.84 (m, 1H), 1.33-1.26 (m, 12H), 1.19 (t, J=7.1 Hz, 3H). ³¹P NMR (162 MHz, methanol-d₄) δ 3.47. ¹⁹F NMR (376 MHz, methanol-d₄) δ −77.75. LCMS: MS m/z=602.97 [M+1], t_{R}=1.45 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.98 min; HPLC system: Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 m/min.

### Example 265. Second Eluting Diastereomer

¹H NMR (400 MHz, methanol-d4) δ 8.00 (s, 1H), 7.37-7.26 (m, 3H), 7.13-7.05 (m, 2H), 6.94 (d, J=4.7 Hz, 1H), 5.55 (d, J=5.2 Hz, 1H), 4.60-4.53 (m, 1H), 4.50-4.43 (m, 2H), 4.42-4.36 (m, 1H), 4.12 (q, J=7.1 Hz, 2H), 3.94-3.82 (m, 1H), 1.34-1.26 (m, 12H), 1.22 (t, J=7.1 Hz, 3H). ³¹P NMR (162 MHz, methanol-d₄) δ 3.47. ¹⁹F NMR (376 MHz, methanol-d₄) δ −77.64. LCMS: MS m/z=602.97 [M+1], t_{R}=1.40 min; LC system: Thermo Accela 1250 UHPLC; MS system: Thermo LCQ Fleet; Column: Kinetex 2.6μ XB-C18 100 A, 50×4.6 mm; Solvents: acetonitrile with 0.1% acetic acid, water with 0.1% acetic acid; Gradient: 0 min-2.0 min 2-100% acetonitrile, 2.0 min-3.05 min 100% acetonitrile, 3.05 min-3.2 min 100%-2% acetonitrile, 3.2 min-3.5 min 2% ACN at 2 μL/min. HPLC: t_{R}=2.94 min; Agilent 1290 II; Column: Phenomenex Kinetex C18, 2.6 u 110 A, 100×4.6 mm; Solvents: A: Water with 0.1% TFA, B: Acetonitrile with 0.1% TFA; Gradient: 2-98% B with 8.5 min gradient at 1.5 mL/min.

### C. Biological Examples

### Example 266. DENV Pol IC50

A 244 nucleotide secondary structureless heteropolymeric RNA (sshRNA) with sequence 5′-(UCAG)20(UCCAAG)14(UCAG)2-3′ is used as the template with 5′-CUG-3′ primer in the DENV2-NS5 polymerase assay. Six two-fold dilutions of compounds starting from 200 nM and no inhibitor control are plated in 96-well plates. 100 nM DENV2 NS5 is preincubated for 5 minutes at room temperature in a reaction mixture containing 40 mM Tris-HCl (pH 7.5), 10 mM NaCl, 3 mM DTT, 0.2 unit/μL RNasin Plus RNase Inhibitor, 200 ng/μL sshRNA, 20 μM CUG and 2 mM MgCl₂. Enzyme mix is added to compound dilutions and reactions initiated by the addition of a mixture containing 20 μM of three natural NTP plus 2 μM of analog:base matched competing natural NTP containing 1:100 α-33P-NTP. After 90 minutes at 30° C., 5 μL of the reaction mixtures are spotted on DE81 anion exchange paper. Filter papers are washed three times with Na₂HPO₄ (125 mM, pH 9) for 5 minutes, rinsed with water and ethanol, then air-dried and exposed to phosphorimager. Synthesized RNA is quantified using Typhoon Trio Imager and Image Quant TL Software and reaction rates are calculated by linear regression using GraphPad Prism 5.0. IC50 values are calculated in Prism by non-linear regression analysis using the dose-response (variable slope) equation (four-parameter logistic equation): Y=Bottom+(Top−Bottom)/(1+10\hat{ }((Log IC₅₀−X)*HillSlope)).

### Example 267. RSV RNP Preparation

RSV ribonucleoprotein (RNP) complexes were prepared from a method modified from Mason et al (1). HEp-2 cells were plated at a density of 7.1×10⁴ cells/cm² in MEM+10% fetal bovine serum (FBS) and allowed to attach overnight at 37° C. (5% CO₂). Following attachment, the cells were infected with RSV A2 (MOI=5) in 35 mL MEM+2% FBS. At 20 hours post-infection, the media was replaced with MEM+2% FBS supplemented with 2 μg/mL actinomycin D and returned to 37° C. for one hour. The cells were then washed once with PBS and treated with 35 mL of PBS+250 μg/mL lyso-lecithin for one minute, after which all liquid was aspirated. The cells were harvested by scrapping them into 1.2 mL of buffer A [50 mM TRIS acetate (pH 8.0), 100 mM potassium acetate, 1 mM DTT and 2 μg/mL actinomycin D] and lysed by repeated passage through an 18 gauge needle (10 times). The cell lysate was placed in ice for 10 minutes and then centrifuged at 2400 g for 10 minutes at 4° C. The supernatant (S1) was removed and the pellet (P1) was disrupted in 600 μL of Buffer B [10 mM TRIS acetate (pH 8.0), 10 mM potassium acetate and 1.5 mM MgCl₂] supplemented with 1% Triton X-100 by repeated passage through an 18 gauge needle (10 times). The resuspended pellet was placed in ice for 10 minutes and then centrifuged at 2400 g for 10 minutes at 4° C. The supernatant (S2) was removed and the pellet (P2) was disrupted in 600 μL of Buffer B supplemented with 0.5% deoxycholate and 0.1% Tween 40. The resuspended pellet was placed in ice for 10 minutes and then centrifuged at 2400 g for 10 minutes at 4° C. The supernatant (S3) fraction, containing the enriched RSV RNP complexes, was collected and the protein concentration determined by UV absorbance at 280 nm. Aliquoted RSV RNP S3 fractions were stored at −80° C.

### Example 268. RSV RNP Assay

Transcription reactions contained 25 μg of crude RSV RNP complexes in 30 μL of reaction buffer [50 mM TRIS-acetate (pH 8.0), 120 mM potassium acetate, 5% glycerol, 4.5 mM MgCl₂, 3 mM DTT, 2 mM ethyleneglycol-bis(ethylether)-tetraacetic acid (EGTA), 50 μg/mL BSA, 2.5 U RNasin (Promega), ATP, GTP, UTP, CTP and 1.5 uCi [α-32P] NTP (3000 Ci/mmol)]. The radiolabeled nucleotide used in the transcription assay was selected to match the nucleotide analog being evaluated for inhibition of RSV RNP transcription. Cold, competitive NTP was added at a final concentration of one-half its Km (ATP=20 μM, GTP=12.5 μM, UTP=6 μM and CTP=2 μM). The three remaining nucleotides were added at a final concentration of 100 μM.

To determine whether nucleotide analogs inhibited RSV RNP transcription, compounds were added using a 6 step serial dilution in 5-fold increments. Following a 90 minute incubation at 30° C., the RNP reactions were stopped with 350 μL of Qiagen RLT lysis buffer and the RNA was purified using a Qiagen RNeasy 96 kit. Purified RNA was denatured in RNA sample loading buffer (Sigma) at 65° C. for 10 minutes and run on a 1.2% agarose/MOPS gel containing 2 M formaldehyde. The agarose gel was dried and exposed to a Storm phosphorimager screen and developed using a Storm phosphorimager (GE Healthcare). The concentration of compound that reduced total radiolabeled transcripts by 50% (IC₅₀) was calculated by non-linear regression analysis of two replicates.

### Example 269. DENV-2 moDC EC50

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and suspended in serum-free RPMI. moDCs were infected with Vero-derived Dengue 2, New Guinea strain (NGC) at a MOI=0.1 for two hours in serum-free RPMI with gentle agitation at 37° C. Cells were washed and resuspended in 10% serum-containing RPMI (Gibco, supplemented with sodium pyruvate, NEAA, Penicillin-Streptomycin). 10\hat{ }5 cells were plated in triplicate in 96-well plates with compounds dispensed at graded doses (Hewlett-Packard D300 Digital Dispenser). All wells were normalized to 0.25% DMSO. At 48 hours, cells were washed with 1×PBS and all supernatants removed. Total RNA was extracted using RNEasy 96 plates (Qiagen) and used to generate first-strand cDNA using XLT cDNA 5× Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example 270. moDC CC50

Human monocyte-derived dendritic cells (moDCs) were derived from CD14+ monocytes (AllCells) cultured in Human Mo-DC Differentiation medium containing GM-CSF and IL-4 (Miltenyi Biotec). On day 7, moDCs were harvested by mechanical disruption, washed and cultured in triplicate at 1×10\hat{ }5-5×10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example 271. DENV-2 Huh-7 EC50

Huh7 (Human hepatocarcinoma 7) cells were maintained in 10% FCS-containing DMEM complete media. On the day of the assay, cells were trypsinized (0.1% Trypsin-EDTA), washed and infected for 2 hours in serum-free DMEM with Dengue serotype 2 New Guinea C (NGC) strain at MOI=0.1 with gentle agitation at 37° C. After 2 hours, cells were washed with serum-free media and suspended in 10% FCS-containing DMEM (Gibco, supplemented with sodium pyruvate, NEAA, Penicillin-Streptomycin). 10\hat{ }5 cells were plated in triplicate in 96-well plates with compounds dispensed at graded doses (Hewlett-Packard D300 Digital Dispenser). All wells were normalized to 0.25% DMSO. At 48 hours, cells were washed with 1×PBS and all supernatants removed. Total RNA was extracted using RNEasy 96 plates (Qiagen) and used to generate first-strand cDNA using XLT cDNA 5×Supermix (QuantaBio). cDNA was used as a template in a Taqman qPCR duplex reaction specific to DENV2 viral and GAPDH gene expression. EC₅₀ values were determined using Prism Graphpad software, with normalization to a positive control and no compound negative control wells.

### Example 272. Huh-7 CC50

Human hepatocarcinoma 7 (Huh7) cells were maintained in 10% FCS-containing complete DMEM. On day of assay, cells were trypsinized with 0.1% Trypsin-EDTA, washed and cultured in triplicate at 1-2×10\hat{ }4 cells/well in 96-well plates with compounds dispensed at graded doses (Hewlett-Packard D300 Digital Dispenser). All wells were normalized to 0.25% DMSO. After 48 hours, CellTiter Glo (Promega) was added and incubated for 10 minutes at room temp before reading on a luminometer. % viability curves were calculated against no compound and no cell control wells. CC₅₀ values were determined using Prism Graphpad software.

### Example 273. RSV HEp-2 EC50

Antiviral activity against RSV is determined using an infectious cytopathic cell protection assay in HEp-2 cells. In this assay, compounds inhibiting viral infection and/or replication produce a cytoprotective effect against the virus-induced cell killing that can be quantified using a cell viability reagent. The techniques used here are novel adaptations of methods described in published literature (Chapman et al., Antimicrob Agents Chemother. 2007, 51(9):3346-53.)

HEp-2 cells are obtained from ATCC (Manassas, VI) and maintained in MEM media supplemented with 10% fetal bovine serum and penicillin/streptomycin. Cells are passaged twice a week and kept at subconfluent stage. Commercial stock of RSV strain A2 (Advanced Biotechnologies, Columbia, MD) is titered before compound testing to determine the appropriate dilution of the virus stock that generates desirable cytopathic effect in HEp-2 cells.

For antiviral tests, HEp-2 cells are grown in large cell culture flasks to near confluency but not fully so. The compounds to be tested are prediluted in DMSO in 384-well compound dilution plates, either in an 8 or 40 sample per plate standardized dose response format. 3-fold serial dilution increments of each test compound are prepared in the plates and test samples are transferred via acoustic transfer apparatus (Echo, Labcyte) at 100 nL per well into cell culture assay 384-well plates. Each compound dilution is transferred in single or quadruplicate samples into dry assay plates, which are stored until assay is ready to go. The positive and negative controls are laid out in opposite on ends of the plate in vertical blocks (1 column).

Subsequently, an infectious mixture is prepared using an appropriate dilution of virus stock previously determined by titration with cells at a density of 50,000/ml and 20 L/well is added to test plates w/compounds via automation (uFlow, Biotek). Each plate includes negative and positive controls (16 replicates each) to create 0% and 100% virus inhibition standards, respectively. Following the infection with RSV, testing plates are incubated for 4 days in a 37° C. cell culture incubator. After the incubation, a cell viability reagent, Cell TiterGlo (Promega, Madison, WI) is added to the assay plates, which are incubated briefly, and a luminescent readout is measured (Envision, Perkin Elmer) in all the assay plates. The RSV-induced cytopathic effect, percentage inhibition, is determined from the levels of remaining cell viability. These numbers are calculated for each tested concentration relative to the 0% and 100% inhibition controls, and the EC₅₀ value for each compound is determined by non-linear regression as a concentration inhibiting the RSV-induced cytopathic effect by 50%. Various potent anti-RSV tool compounds are used as positive controls for antiviral activity.

### Example 274. HEp-2 CC50

Cytotoxicity of tested compounds is determined in uninfected HEp-2 cells in parallel with the antiviral activity using the cell viability reagent in a similar fashion as described before for other cell types (Cihlar et al., Antimicrob Agents Chemother. 2008, 52(2):655-65). The same protocol as for the determination of antiviral activity is used for the measurement of compound cytotoxicity except that the cells are not infected with RSV. Instead, an uninfected cell mixture at the same density is added at 20 ul/well to plates containing prediluted compounds, also at 100 nL/sample. Assay plates are then incubated for 4 days followed by a cell viability test using the same CellTiter Glo reagent addition and measurement of luminescent readouts. Untreated cell and cells treated at 2 μM puromycin (Sigma, St. Louis, MO) serve as 100% and 0% cell viability control, respectively. The percent of cell viability is calculated for each tested compound concentration relative to the 0% and 100% controls and the CC₅₀ value is determined by non-linear regression as a compound concentration reducing the cell viability by 50%.

### Example 275. RSV NHBE EC50

Normal human bronchial epithelial (NHBE) cells were purchased from Lonza (Walkersville, MD, Cat #CC-2540) and cultured in Bronchial Epithelial Growth Media (BEGM) (Lonza, Walkersville, MD, Cat #CC-3170). The cells were passaged 1-2 times per week to maintain <80% confluency. The NHBE cells were discarded after 6 passages in culture.

To conduct the RSV A2 antiviral assay, NHBE cells were plated in 96-well plates at a density of 7,500 cells per well in BEGM and allowed to attach overnight at 37° C. Following attachment, 100 μL of cell culture media was removed and 3-fold serially diluted compound was added using a Hewlett-Packard D300 Digital Dispenser. The final concentration of DMSO was normalized to 0.05%. Following compound addition, the NHBE cells were infected by the addition of 100 μL of RSV A2 at a titer of 1×10^{4.5} tissue culture infectious doses/mL in BEGM and then incubated at 37° C. for 4 days. The NHBE cells were then allowed to equilibrate to 25° C. and cell viability was determined by removing 100 μL of culture medium and adding 100 μL of Cell-Titer Glo viability reagent. The mixtures were incubated for 10 minutes at 25° C., and the luminescence signal was quantified on an Envision luminescence plate reader.

### Example 276. RSV HAE EC₅₀

HAE cells are cultured at the air-liquid interface and have an apical side that is exposed to the air and a basal side that is in contact with the medium. Prior to experimentation, HAE were removed from their agar-based shipping packaging and were acclimated to 37° C./5% CO2 overnight in 1 ml of HAE Assay medium (AIR-100-MM, Mattek Corp). HAE were prepared for infection by washing the apical surface twice with 400 μL of PBS (either utilizing direct pipetting methods or by running each transwell through a trough containing PBS) to remove the mucus layer. Apical chambers were drained of PBS and tapped gently onto absorbent material to remove as much PBS as possible. After washing, the cells were transferred to fresh HAE maintenance media containing 4-fold serially diluted compound, delivered to the basal side of the cell monolayer, and apically infected with 100 μL of a 1:600 dilution of RSV A strain A2 1000× stock (ABI, Columbia, MD, cat #10-124-000) in HAE assay medium for 3 hours at 37° C. in 5% CO2. The virus inoculum was removed and the apical surface of the cells was washed 3 times with PBS using either method previously described. The cells were then cultured in the presence of compound for 3 days at 37° C. Following the incubation, total RNA was extracted from the HAE cells using a MagMAX-96 Viral RNA isolation kit (Applied Biosystems, Foster City, CA, Cat #AM1836) and intracellular RSV RNA was quantified by real-time PCR. Approximately 25 ng of purified RNA was added to a PCR reaction mixture that contained 0.9 μM RSV N Forward and RSV N Reverse primers, 0.2 μM RSV N Probe and 1×Taqman RNA-to-Ct 1-Step Kit (Applied Biosystems, Foster City, CA, Cat #4392938). RNA levels were normalized using a Tagman GAPDH control primer set (Applied Biosystems, Foster City, CA, Cat #402869). Real-Time PCR Primers and Probe Used in the RSV A2 HAE Antiviral Assay:

RSV N Forward CATCCAGCAAATACACCATCCA,
RSV N Reverse TTCTGCACATCATAATTAGGAGTATCAA,
RSV N Probe FAM-CGGAGCACAGGAGAT-BHQ

### Example 277. HRV16 HELA EC₅₀

H1-HeLa cells, cultured in complete DMEM medium containing 10% heat-inactivated FBS and 1% Penicillin/Streptomycin, were seeded in 96 well plates at 3000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and the appropriate dilution of virus stock, previously determined by titration and prepared in cell culture media, was added to test plates containing cells and serially diluted compounds. Each plate included 6 wells of infected untreated cells and 6 wells of uninfected cells that served as 0% and 100% virus inhibition control, respectively. Following the infection, test plates were incubated for 96 h in a tissue culture incubator set to 33° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example 278. HRV1A HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV1a virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example 279. HRV14 HELA EC₅₀

H1-HeLa cells, cultured in complete RPMI 1640 medium containing 10% heat-inactivated FBS and 1% Penicillin/Streptomycin, were seeded in 96 well plates at 5000 cells/well one day prior to compound dosing and infection. The antiviral activity of each compound was measured in triplicate. Compounds were added directly to the cell cultures in serial 3-fold dilutions using the HP300 digital dispenser (Hewlett Packard, Palo Alto, CA) immediately prior to infection. The plates were transferred to BSL-2 containment and 100 μL of 1/4000 dilution of HRV14 virus stock was added to each well containing cells and serially diluted compounds. Each plate included 6 wells of infected untreated cells and 6 wells of cells containing 5 μM Rupintrivir that served as 0% and 100% virus inhibition control, respectively. Following the infection, test plates were incubated for 96 h in a tissue culture incubator set to 37° C./5% CO₂. Following incubation, the H1-HeLa cells were removed from incubation and allowed to equilibrate to 25° C. Cell viability was determined by removing 100 μL of culture medium and adding 100 μL of Cell-Titer Glo viability reagent. The mixtures were incubated on a shaker for 10 minutes at 25° C., and the luminescence signal was quantified on an Envision luminescence plate reader. The percentage inhibition of virus infection was calculated for each tested concentration relative to the 0% and 100% inhibition controls and the EC₅₀ value for each compound was determined by 4-parametric non-linear regression as the effective concentration of compound that inhibited cytopathic effect by 50%.

### Example 280. HRVc15 and HRVc25 EC₅₀

First, HRV replicon RNA is prepared. 5 ug of DNA Template (HRVc15 or HRVc25) is linearized with 2 μL of MluI enzyme in NEB buffer-3 in a final volume of 25 μL for 3 hours at 37° C. Following incubation, linearized DNA is purified on a PCR purification column and the following in vitro transcription is performed using the following conditions: 10 μL of RiboMAX Express T7 2× buffer, 1-8 μL of linear DNA template (1 μg), 0-7 μL nuclease free water, 2 μL enzyme mix T7 express. The final volume of 20 μL is mixed and incubated at 37° C. for 30 min. Following incubation, 1 μL of RQ1 RNase free DNase is added and the mixture is incubated at 37° C. for 15 min. The resulting RNA is then purified with the MegaClear Kit (Gibco Life Technologies cat #11835-030) and is eluted two times with 50 μL of elution buffer at 95° C. H1-HeLa cells cultured in complete RPMI 1640 media containing 10% heat-inactivated FBS and 1% Penicillin/Streptomycin are seeded into T-225 flasks at a concentration of 2E6 cells/flask a day prior to transfection and are incubated at 37° C./5% CO₂ overnight. On the day of transfection, cells are trypsinized following standard cell culture protocols and are washed two times with PBS. Following washes, cells are resuspended at a concentration of 1E7 cells/mL in PBS and the suspension is stored on wet ice. Electroporation is used to introduce replicon RNA into the H1-HeLa cells. A final volume of 10 μL containing 10 μg of replicon c15 or 1 μg of c25 replicon RNA, respectively, are pipetted into a 4 mm electroporation cuvette. The H1-HeLa cell stock is mixed by gently swirling and 0.5 mL of the cell stock previously prepared is transferred into the cuvette containing the replicon RNA. The combined solution is flicked to mix. Following mixing, cells are immediately electroporated using the following settings: 900V, 25 uF, infinite resistance, 1 pulse. Cuvettes are rested on ice for 10 min. Following the 10 min incubation, add 19 mL of ambient temperature, phenol red-free and antibiotic-free RPMI 1640 containing 10% heat-inactivated FBS per electroporation. 150 μL (4E4 cells) of the electroporated cell suspension are seeded per well into a 96 well clear-bottom, white cell culture plate, and are incubated at 25° C. for 30 min. Compounds were added directly to the cell cultures in serial 3-fold dilutions using the HP300 digital dispenser (Hewlett Packard, Palo Alto, CA) and were tested in triplicate. Following the addition of compounds, plates are incubated at 33° C. for 48 hrs. Replicon activity is then measured by a Renilla-Glo Luciferase Assay system. Prior to signal quantification, plates are removed from incubators and are allowed to equilibrate to 25° C. after 50 uL is removed from each well. Following manufacturer's protocol, a 1:100 dilution of Renilla-Glo substrate to buffer is prepared and 100 uL of the Renill-Glo luciferase mix is added to each well. Plates are then incubated for 20 min at 25° C. under gentle agitation and luciferase signal are determined with a 0.1 second detection setting using an EnVision luciferase quantification reader. The percentage inhibition of replicon inhibition was calculated for each tested concentration relative to the 0% and 100% inhibition controls included in the experiments and the EC₅₀ value for each compound was determined by 4-parametric non-linear regression as the effective concentration of compound that inhibited luciferase signal by 50%.

### Example 281. DENV-2 Huh-7 Rep EC₅₀

In 384 well plates (Greiner, Cat #781091), compounds were acoustically transferred at 200 nl per well in a 8 compound (4 replicates) or 40 compound dose response format (3 replicates). For all plates tested, Balapiravir, GS-5734 and NITD008 were included as positive inhibition controls alongside 0% inhibition, DMSO-only negative control wells. Following compound addition, Huh-7 cells containing the DENV2 replicon construct were harvested following standard cell culture procedures and were adjusted to a concentration of 1.25E5 cells/mL in cell culture media composed of cDMEM without genticin. 40 μL of the cell stock was then added to each well for a final cell density of 5,000 cells/well. Cell and compound mixtures were incubated at 37° C./5% CO₂ for 48 hours. Prior to harvesting cells, EnduRen Live Cell Substrate (Promega, Cat #E6481) was prepared by suspending 3.4 mg into 100 uL of DMSO to generate a 60 mM stock solution. The stock solution was then diluted 1:200 in pre-warmed cDMEM and 10 uL of this diluted solution was added to each well of the 384 well plates. Plates were then centrifuged at 500 rpm briefly and were placed on a plate shaker for 2 min. Following mixing, plates were incubated at 7° C./5% CO₂ for 1.5 hours prior to measuring luminescence on an Envision luminometer. The percentage inhibition of replicon signal was calculated for each tested concentration relative to the 0% and 100% inhibition controls and the EC₅₀ value for each compound was determined by 4-parametric non-linear regression as the effective concentration of compound that inhibited replicon signal by 50%.

### Example 282. HCV Rep 1B and 2A EC₅₀

Compounds were serially diluted in ten steps of 1:3 dilutions in 384-well plates. All serial dilutions were performed in four replicates per compound within the same 384-well plate. An HCV protease inhibitor ITMN-191 at 100 μM was added as a control of 100% inhibition of HCV replication while puromycin at 10 mM was included as a control of 100% cytotoxicity. To each well of a black polystyrene 384-well plate (Greiner Bio-one, Monroe, NC), 90 μL of cell culture medium (without Geneticin) containing 2000 suspended HCV replicon cells was added with a Biotek μFlow workstation. For compound transfer into cell culture plates, 0.4 μL of compound solution from the compound serial dilution plate was transferred to the cell culture plate on a Biomek FX workstation. The DMSO concentration in the final assay wells was 0.44%. The plates were incubated for 3 days at 37° C. with 5% CO₂ and 85% humidity. The HCV replicon assay was a multiplex assay, able to assess both cytotoxicity and antireplicon activity from the same well. The CC₅₀ assay was performed first. The media in the 384-well cell culture plate was aspirated, and the wells were washed four times with 100 μL of PBS each, using a Biotek ELX405 plate washer. A volume of 50 μL of a solution containing 400 nM calcein AM (Anaspec, Fremont, CA) in 1×PBS was added to each well of the plate with a Biotek μFlow workstation. The plate was incubated for 30 min at room temperature before the fluorescence signal (excitation 490 nm, emission 520 nm) was measured with a Perkin-Elmer Envision plate reader. The EC₅₀ assay was performed in the same wells as the CC₅₀ assay. The calcein-PBS solution in the 384-well cell culture plate was aspirated with a Biotek ELX405 plate washer. A volume of 20 μL of Dual-Glo luciferase buffer (Promega, Madison, WI) was added to each well of the plate with a Biotek μFlow Workstation. The plate was incubated for 10 min at room temperature. A volume of 20 μL of a solution containing a 1:100 mixture of Dual-Glo Stop & Glo substrate (Promega, Madison, WI) and Dual-Glo Stop & Glo buffer (Promega, Madison, WI) was added to each well of the plate with a Biotek μFlow Workstation. The plate was then incubated at room temperature for 10 min before the luminescence signal was measured with a Perkin-Elmer Envision Plate Reader.

### Example 283. HEp-2 and MT4 CC₅₀

Cytotoxicity of the compounds was determined in uninfected cells using the cell viability reagent in a similar fashion as described before for other cell types (Cihlar et al., Antimicrob Agents Chemother. 2008, 52(2):655-65). HEp-2 (1.5×103 cells/well) and MT-4 (2×103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 100% and 0% cell viability control, respectively. The percent of cell viability was calculated for each tested compound concentration relative to the 0% and 100% controls and the CC₅₀ value was determined by non-linear regression as a compound concentration reducing the cell viability by 50%.

TABLE 3
Activity

| | RSV | RSV | RSV | HRV16 |
|---|---|---|---|---|
| | HEp-2 | NHBE | HAE | HELA |
| Example | EC₅₀ | EC₅₀ | EC₅₀ | EC₅₀ |
| No. | (nM) | (nM) | (nM) | (nM) |
| 1 | 342 | 879 | | 966 |
| 2 | 56 | 122 | | 353 |
| 3 | 4094 | 1688 | | 8956 |
| 4 | 96 | 76 | | 510 |
| 5 | 2735 | 630 | | 654 |
| 6 | 88 | 124 | 20 | 227 |
| 7 | 1862 | 265 | | 5938 |
| 8 | 182 | 351 | | 472 |
| 9 | 1006 | 97 | | 324 |
| 10 | 191 | 421 | | 583 |
| 11 | 269 | 54 | | 1202 |
| 12 | 244 | 29 | | 1926 |
| 13 | 93 | 30 | | |
| 14 | 3646 | 1270 | | 12449 |
| 15 | 4329 | 3255 | | 11443 |
| 16 | 1292 | 104 | | 1307 |
| 17 | 308 | 88 | | 489 |
| 18 | 114 | 91 | 27 | 212 |
| 20 | 877 | 2233 | | 4875 |
| 21 | 79 | 27 | | 230 |
| 22 | 103 | 36 | 19 | 200 |
| 23 | 207 | 34 | | |
| 25 | 26 | 127 | 152 | 135 |
| 26 | 334 | 25 | | 669 |
| 27 | 1303 | 18 | | 3155 |
| 28 | 238 | 53 | | 590 |
| 29 | 91 | 932 | | 678 |
| 30 | 844 | 44 | | |
| 31 | 334 | 258 | | 2496 |
| 32 | 3438 | >3124. | | 9946 |
| 33 | 1914 | 2682 | | 26973 |
| 34 | 14004 | 2838 | | >50000 |
| 35 | 4066 | 1308 | | 29560 |
| 36 | 1439 | 312 | | 10715 |
| 37 | 136 | 129 | | 329 |
| 38 | 3507 | 3978 | | 20895 |
| 39 | 166 | 569 | | 1082 |
| 40 | 21481 | 5000 | | >50000 |
| 41 | >50000 | 5000 | | >50000 |
| 42 | 25389 | 5000 | | >50000 |
| 43 | 55345 | 5000 | | |
| 44 | 998 | 1799 | | 4260 |
| 45 | 55 | 248 | | 375 |
| 46 | 82 | 393 | | 487 |
| 47 | 51 | 162 | | 560 |
| 49 | 1076 | 2429 | | 8720 |
| 50 | 44 | 498 | | 464 |
| 51 | 90 | >1244 | | 357 |
| 52 | 94 | 252 | | 1254 |
| 53 | 2359 | 5000 | | 15534 |
| 54 | | 105 | | 378 |
| 55 | 570 | 4295 | | 6908 |
| 56 | 4174 | 2241 | | 7957 |
| 57 | 1485 | >4183 | | 5476 |
| 58 | | 917 | | |
| 59 | | 111 | | |
| 60 | 583 | 361 | | |
| 61 | 2286 | 5000 | | 23164 |
| 62 | 4525 | >5000 | | 8569 |
| 63 | 4073 | >5000 | | |
| 64 | 350 | 3502 | | |
| 65 | | | | |
| 66 | | | | |
| 67 | | | | |
| 68 | 247 | | | 4719 |
| 69 | | | | 7325 |
| 70 | | | | 5232 |
| 71 | 3142 | >5000 | | |
| 75 | 1262 | | | |
| 75 | | | | |
| 77 | 464 | 1150 | | 1419 |
| 78 | 2045 | 1766 | | 2262 |
| 79 | >50000 | >5000 | | >34792 |
| 80 | 3827 | 4700 | | 8962 |
| 81 | 3566 | 2646 | | 3374 |
| 82 | 2509 | 3000 | | 9270 |
| 83 | 5300 | 953 | | |
| 84 | 8067 | 1133 | | 19943 |
| 85 | >46697 | >5000 | | >50000 |
| 86 | 22607 | >5000 | | >50000 |
| 87 | 1343 | 1697 | | 6707 |
| 88 | 336 | 36 | | 1059 |
| 89 | 9551 | 713 | | |
| 90 | 516 | 208 | | 1047 |
| 91 | 12110 | >5000 | | >50000 |
| 92 | 64 | 73 | | 225 |
| 93 | 2839 | 85 | | |
| 94 | 100 | 118 | | 209 |
| 95 | 15167 | >5000 | | >42218 |
| 96 | 254 | 441 | 22361 | 9354 |
| 97 | 537 | 1368 | | 4425 |
| 98 | >50000 | >5000 | | >50000 |
| 99 | >50000 | >5000 | | >50000 |
| 100 | >5600 | 3599 | | 17955 |
| 101 | 1744 | 1239 | | 3129 |
| 102 | 9945 | >5000 | | 16724 |
| 103 | 938 | 548 | | 2438 |
| 104 | 790 | 368 | | 1799 |
| 105 | 3443 | 2018 | | 16937 |
| 106 | >42532 | >5000 | | >100000 |
| 107 | >14223 | >2889 | | >35721 |
| 108 | 147 | 239 | | 289 |
| 109 | 1074 | 452 | | 2493 |
| 110 | 281 | 339 | | |
| 111 | 3967 | >1482 | | 25305 |
| 112 | 1406 | 464 | | 3685 |
| 113 | 611 | 352 | | 2637 |
| 114 | 974 | 255 | | 4696 |
| 115 | 8663 | 1553 | | 10295 |
| 116 | >100000 | >5000 | | >31940 |
| 117 | >100000 | >5000 | | >100000 |
| 118 | 214 | 316 | | 424 |
| 119 | 539 | 120 | | 247 |
| 120 | 101 | 104 | | 295 |
| 121 | >100000 | >5000 | | >100000 |
| 122 | 4036 | >5000 | | 29320 |
| 123 | >100000 | >5000 | | >100000 |
| 124 | >100000 | >5000 | | >86429 |
| 125 | 3304 | 3647 | | 7646 |
| 126 | 6557 | 1787 | | 27922 |
| 127 | 1728 | 434 | | |
| 128 | 1871 | 164 | | |
| 129 | 42911 | >5000 | | 41611 |
| 130 | 3588 | 438 | | 7305 |
| 131 | 315 | 636 | | 1806 |
| 132 | 1993 | 523 | | 22356 |
| 133 | 4720 | >5000 | | 16529 |
| 134 | 334 | 52 | | 247 |
| 135 | 233 | 66 | | |
| 136 | 2774 | 95 | | 577 |
| 137 | 3693 | >5000 | | 26953 |
| 138 | 38498 | >5000 | | |
| 139 | 6434 | 103 | | 28269 |
| 140 | 8888 | >5000 | | 23277 |
| 141 | 2012 | 67 | | 14924 |
| 142 | 251 | 61 | | 164 |
| 143 | 142 | 53 | 157 | |
| 144 | 614 | 65 | | |
| 145 | 2895 | >5000 | | 46167 |
| 146 | 9716 | >5000 | | >50000 |
| 148 | 2670 | 320 | | 36915 |
| 149 | 925 | >891 | 188 | >50000 |
| 150 | 4158 | >5000 | | 49889 |
| 152 | >5000 | 1780 | | >5000 |
| 153 | 7141 | >5000 | | 12469 |
| 154 | 2702 | 311 | | 1950 |
| 155 | 4319 | 1578 | | 4199 |
| 156 | 8937 | 1368 | | 25872 |
| 157 | 972 | 163 | | |
| 158 | 969 | 1269 | | 13012 |
| 159 | 220 | 489 | | 1277 |
| 160 | 7298 | >5000 | | 29145 |
| 161 | 6476 | 1493 | | 13781 |
| 162 | 6055 | >5000 | | 12850 |
| 163 | 13258 | 9213 | | >50000 |
| 164 | 1223 | 724 | | 3995 |
| 165 | 1944 | 1608 | | 11329 |
| 166 | 83 | 624 | | 336 |
| 167 | 647 | 359 | | 11953 |
| 168 | 53 | 103 | | 160 |
| 169 | 524 | 965 | | |
| 170 | 164 | 524 | | 17526 |
| 171 | 220 | 375 | | 582 |
| 172 | 1450 | >516 | | |
| 173 | 156 | 282 | 22 | |
| 174 | 257 | 1387 | | 748 |
| 175 | 801 | 539 | | |
| 176 | 161 | 1637 | | 547 |
| 177 | 104 | 1559 | | 1158 |
| 178 | | | | |
| 179 | 591 | >4805 | | |
| 180 | 142 | 833 | | 851 |
| 181 | 8 | 43 | 212 | 35 |
| 182 | 1166 | 2087 | | 8563 |
| 183 | 80 | 1202 | | 173 |
| 185 | 7935 | >5000 | | >80205 |
| 186 | 2774 | 70 | | 4748 |
| 187 | 1485 | >5000 | | 20563 |
| 188 | 186 | 38 | | 429 |
| 190 | 136 | 2063 | | 2637 |
| 191 | 2385 | 35 | 11 | 3403 |
| 192 | 194 | 21 | | 342 |
| 193 | 244 | 469 | | 750 |
| 194 | | | | |
| 195 | 2590 | 678 | | 2547 |
| 196 | 4134 | 1322 | | 7537 |
| 198 | 618 | 37 | | 548 |
| 199 | 1118 | 33 | | 738 |
| 200 | 1404 | 125 | | 5680 |
| 201 | 2518 | 3096 | | 10295 |
| 202 | 7046 | >2948 | | |
| 203 | 31 | 18 | | 72 |
| 204 | 463 | 628 | | 1377 |
| 205 | 101 | 27 | | 417 |
| 206 | 85 | 62 | | |
| 207 | 124 | 91 | | 183 |
| 208 | >32800 | >4216 | | >50000 |
| 209 | 1463 | >5000 | | 16695 |
| 210 | 1775 | 3018 | | 27943 |
| 212 | 133 | >5000 | | 670 |
| 213 | 3841 | >5000 | | |
| 214 | 2936 | 551 | | 25823 |
| 215 | 1103 | >3002 | | 3390 |
| 216 | 4233 | >4393 | | 32750 |
| 217 | 1009 | >3513 | | 4656 |
| 218 | 1156 | 280 | | 11320 |
| 219 | 1762 | 178 | | 6044 |
| 220 | 4264 | 340 | | 10564 |
| 221 | 1177 | >1053 | | 8492 |
| 222 | 4452 | 1624 | | |
| 223 | | 321 | | |
| 224 | | 2834 | | |
| 225 | | | | |
| 226 | 1912 | 2549 | | |
| 227 | 614 | 126 | | 4936 |
| 228 | | 2042 | | 4715 |
| 229 | >13322 | >5000 | | >40184 |
| 230 | 1649 | >5000 | | 11512 |
| 231 | 152 | 190 | | 1199 |
| 232 | 61 | 98 | | 479 |
| 233 | 1746 | >4640 | | 3346 |
| 234 | 696 | 2410 | | 6868 |
| 235 | 114 | 510 | | 1104 |
| 236 | 189 | 949 | | 741 |
| 237 | >38425 | >10772 | | >50000 |
| 238 | 91 | 41 | | 731 |
| 239 | 148 | 54 | | 1123 |
| 240 | 166 | 190 | | 1308 |
| 241 | 293 | 190 | | |
| 242 | | | | |
| 243 | | | | |
| 244 | 4946 | >5000 | | 33561 |
| 245 | 3790 | >5000 | | 28981 |
| 246 | 35 | 41 | | |
| 247 | 44 | 510 | | 317 |
| 248 | 26 | 2619 | | 174 |
| 249 | | 5000 | | |
| 250 | 69 | 51 | | 1175 |
| 251 | 8 | | | |
| 252 | 26 | 31 | | |
| 253 | 20 | 70 | | |
| 254 | 17 | 46 | | |
| 255 | 18 | 43 | | |
| 256 | 54 | 430 | | |
| 257 | 29 | | | |
| 258 | | | | |
| 259 | | | | |
| 260 | 66 | | | |
| 261 | 34 | | | |
| 262 | | | | |
| 263 | 35 | | | |
| 264 | 41 | | | |
| 265 | 215 | | | |

TABLE 4
Activity

| | HRV1A | HRV10 | HRV14 | | | |
|---|---|---|---|---|---|---|
| | HELA | HELA | HELA | HRV99 | HRVc15 | HRVc25 |
| Example | EC₅₀ | EC₅₀ | EC₅₀ | EC₅₀ | EC₅₀ | EC₅₀ |
| No. | (nM) | (nM) | (nM) | (nM) | (nM) | (nM) |
| 2 | 106 | 114 | 340 | 708 | | |
| 4 | 190 | 144 | 445 | 1090 | | |
| 6 | | | | | 9 | 7 |
| 8 | 211 | 179 | 615 | 1337 | | |
| 77 | 736 | 636 | 1807 | 4944 | | |
| 82 | 2777 | 2682 | 9194 | 19322 | | |
| 88 | 581 | 608 | 1694 | 4545 | | |
| 92 | 97 | 81 | 242 | 639 | | |
| 96 | 1584 | 2324 | 12013 | 63935 | | |
| 101 | 1626 | 1311 | 3432 | 7341 | | |
| 103 | 1017 | 916 | 2554 | 4739 | | |

TABLE 5
Activity

| | DENV-2 | DENV-2 | DENV-2 |
|---|---|---|---|
| | Huh-7 Rep | moDC | Huh-7 |
| Example | EC₅₀ | EC₅₀ | EC₅₀ |
| No. | (nM) | (nM) | (nM) |
| 1 | >50000 | | |
| 3 | >100000 | | |
| 6 | 2937 | >6194 | 1633 |
| 7 | 32888 | >25000 | |
| 8 | 37271 | | |
| 9 | >50000 | | 1460 |
| 10 | 17933 | | 2787 |
| 11 | >50000 | | 3500 |
| 12 | >50000 | | |
| 14 | >100000 | | |
| 15 | >50000 | | |
| 16 | 70770 | | |
| 17 | 53718 | | |
| 18 | 12266 | | |
| 20 | >50000 | | |
| 21 | 11023 | | |
| 22 | 5754 | | 665 |
| 25 | 2858 | 524 | 840 |
| 26 | 44291 | | |
| 27 | 82185 | | 5679 |
| 28 | 29633 | | 2155 |
| 29 | 36056 | >5000 | 1885 |
| 30 | 60736 | | |
| 31 | 96002 | | |
| 32 | >100000 | | |
| 33 | >50000 | | |
| 34 | >50000 | | |
| 35 | >50000 | | |
| 36 | >50000 | | |
| 37 | 6700 | | 1509 |
| 38 | >50000 | | |
| 39 | >50000 | | |
| 41 | >50000 | | |
| 42 | 39238 | | |
| 43 | >100000 | | |
| 45 | 9660 | | |
| 46 | >20795 | | |
| 47 | 9188 | | |
| 49 | >50000 | | |
| 50 | 6923 | | |
| 51 | 9211 | | |
| 52 | >46953 | | 5483 |
| 53 | >50000 | | |
| 54 | 7398 | 804 | 2590 |
| 55 | >50000 | | |
| 56 | >50000 | | |
| 57 | >50000 | | |
| 59 | 56512 | | 4652 |
| 60 | 41344 | | 6819 |
| 66 | >100000 | | |
| 69 | >100000 | | |
| 70 | >100000 | | |
| 77 | 49829 | | |
| 78 | >50000 | | |
| 79 | 40170 | | |
| 80 | >50000 | | |
| 81 | 38939 | | |
| 82 | >50000 | | |
| 84 | >50000 | | |
| 85 | >50000 | | |
| 86 | >50000 | | |
| 87 | >100000 | | |
| 88 | 62001 | | >25000 |
| 90 | 44488 | | |
| 91 | >50000 | | |
| 94 | 2336 | 398 | 451 |
| 95 | >50000 | | |
| 96 | >100000 | >25000 | |
| 97 | >100000 | | |
| 98 | >50000 | | |
| 99 | >50000 | | |
| 100 | >50000 | | |
| 101 | >100000 | | |
| 102 | >100000 | | |
| 103 | >50000 | | |
| 104 | >50000 | | |
| 105 | >50000 | | |
| 106 | >100000 | | |
| 107 | >50000 | | |
| 108 | 40807 | | |
| 109 | >50000 | | |
| 111 | >50000 | | |
| 112 | >50000 | | |
| 113 | 42739 | | |
| 114 | >50000 | | |
| 115 | >50000 | | |
| 116 | 11740 | | |
| 117 | >100000 | | |
| 118 | 26976 | | |
| 119 | 31105 | | 4278 |
| 120 | 7458 | | 798 |
| 121 | 56701 | | |
| 122 | >50000 | | |
| 123 | 48460 | | |
| 124 | >100000 | | |
| 125 | >100000 | | |
| 126 | >50000 | | |
| 129 | 26708 | | |
| 130 | >100000 | | |
| 131 | >50000 | | |
| 132 | 44565 | | |
| 133 | >50000 | | |
| 134 | 24708 | | |
| 135 | | | 1391 |
| 136 | 51079 | | |
| 137 | >50000 | | |
| 139 | >50000 | | |
| 140 | >50000 | | |
| 141 | >50000 | | |
| 142 | 12250 | | |
| 145 | >100000 | | |
| 146 | >50000 | | |
| 148 | >50000 | | |
| 149 | >50000 | | |
| 150 | >100000 | | |
| 153 | >100000 | | |
| 154 | 50735 | | |
| 155 | 84391 | | |
| 156 | >100000 | | |
| 158 | >50000 | | |
| 159 | >50000 | | |
| 160 | >50000 | | |
| 161 | >50000 | | |
| 162 | 13758 | | |
| 163 | >50000 | | |
| 164 | >50000 | | |
| 165 | >100000 | | |
| 166 | 22097 | | |
| 167 | >100000 | | |
| 168 | 4673 | | |
| 170 | >50000 | | |
| 171 | 31472 | | |
| 172 | 31390 | | |
| 173 | | | 1207 |
| 174 | 33772 | | |
| 175 | >100000 | | |
| 176 | 28585 | | |
| 177 | >50000 | | |
| 180 | 6615 | | |
| 181 | 5247 | | 795 |
| 182 | >50000 | | |
| 183 | 17271 | | |
| 185 | >100000 | | |
| 186 | 60308 | | |
| 187 | 9353 | | |
| 188 | 21383 | | 3021 |
| 190 | >100000 | | |
| 191 | 61310 | 1073 | 13274 |
| 192 | 15851 | 534 | 2764 |
| 193 | >50000 | | |
| 195 | >100000 | | |
| 198 | 39122 | | |
| 199 | 37789 | | |
| 200 | >50000 | | |
| 201 | >50000 | | |
| 203 | 4460 | 599 | 1732 |
| 204 | 27503 | | |
| 205 | 6103 | | |
| 207 | 4836 | | |
| 208 | >50000 | | |
| 209 | >50000 | | |
| 210 | >50000 | | |
| 212 | 9768 | | |
| 213 | >100000 | | |
| 214 | >50000 | | |
| 215 | 44254 | | |
| 216 | 32998 | | |
| 217 | 37974 | | |
| 218 | >50000 | | |
| 219 | >50000 | | |
| 220 | 28980 | | |
| 221 | >100000 | | |
| 222 | >100000 | | |
| 227 | 39381 | | |
| 229 | 31400 | | |
| 230 | >50000 | | |
| 231 | 14738 | | |
| 232 | 5324 | 1091 | |
| 233 | 35840 | | |
| 234 | 36390 | | |
| 235 | 3958 | | |
| 236 | 17930 | | |
| 237 | >50000 | | |
| 238 | 9708 | | |
| 239 | 25378 | | |
| 240 | 10557 | | |
| 241 | 15455 | | |
| 244 | 36716 | | |
| 245 | >50000 | | |
| 246 | 5114 | | |
| 247 | 12000 | | 1278 |
| 248 | 4725 | | 756 |
| 249 | >50000 | | |
| 251 | 3533 | 116 | 529 |
| 252 | 6996 | 284 | 697 |
| 253 | 2775 | 429 | 489 |
| 254 | 12958 | 90 | 797 |
| 255 | 4945 | 86 | 824 |
| 256 | >100000 | 103 | >18091 |
| 257 | 5482 | | 2208 |
| 260 | 9951 | | 682 |
| 263 | 8030 | | 569 |
| 265 | | | 390 |

TABLE 6
Activity

| | HCV | HCV |
|---|---|---|
| | Rep 1B | Rep 2A |
| Example | EC₅₀ | EC₅₀ |
| No. | (nM) | (nM) |
| 4 | 52675 | 9197 |
| 6 | 793 | 1268 |
| 18 | 1106 | 1485 |
| 25 | 443 | 448 |
| 52 | 3957 | 7457 |
| 60 | 6084 | 10805 |
| 66 | 14852 | 21812 |
| 69 | 25321 | 50301 |
| 70 | 9433 | 32661 |
| 88 | 23664 | 27667 |
| 92 | 16545 | 2905 |
| 94 | 278 | 493 |
| 96 | 30706 | 47256 |
| 149 | 31069 | >44444 |
| 181 | 861 | 1678 |
| 191 | 19748 | 34104 |
| 203 | 332 | 671 |
| 227 | 10188 | 4759 |
| 247 | 665 | 1680 |
| 248 | 503 | 1180 |
| 251 | 595 | 1108 |
| 252 | 624 | 1649 |
| 253 | 452 | 887 |
| 254 | 368 | 787 |

TABLE 7
Activity

| | MT4 | HEp-2 | HELA |
|---|---|---|---|
| Example | CC₅₀ | CC₅₀ | CC₅₀ |
| No. | (nM) | (nM) | (nM) |
| 205 | 20240 | 58553 | 59215 |
| 206 | 16637 | 55177 | |
| 230 | >50000 | >50000 | >50000 |
| 233 | 26355 | >50000 | >50000 |
| 238 | 45010 | >50000 | >50000 |
| 239 | >50000 | >50000 | >50000 |
| 240 | >50000 | >50000 | >50000 |
| 241 | >50000 | >50000 | |
| 250 | 20516 | >50000 | >50000 |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula (Ia): 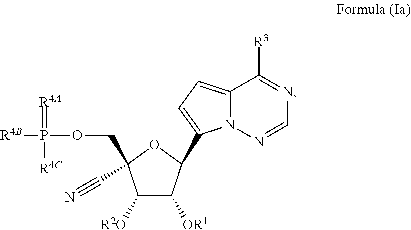
or a pharmaceutically acceptable salt thereof, wherein:
R¹ and R² are each independently H or —C(O)R^{1A}, wherein R^{1A} is C₁₋₆ alkyl, wherein at least one of R¹ and R² is H;
or R¹ and R² are combined to form —C(O)— or —C(R^{2A})(R^{2B})—, wherein each R^{2A} and R^{2B} is independently H, C₁₋₆ alkyl or C₁₋₆ alkoxy;
R³ is —N(H)(R^{3A});
R^{3A} is H or —C(O)R^{3A1}, wherein R^{3A1} is C₁₋₁₈ alkyl optionally substituted with —NH₂;
R^{4A} is O or S; and
R^{4B} and R^{4C} are each independently:
(A) —OH;
(B) —OR^{4B1}, wherein
R^{4B1} is C₁₋₆ alkyl optionally substituted with 1 to 3 R^{4B2} groups, C₁₋₆ haloalkyl, C₃₋₈ cycloalkyl, C₆₋₁₂ aryl, or a 5 to 6 membered heteroaryl having 1 to 3 heteroatoms each independently selected from N, O or S, wherein
each R^{4B2} group is independently C₁₋₆ alkoxy, —S—R^{4B3}, or —S(O)₂—R^{4B3}, and
each R^{4B3} group is independently C₁₋₆ alkyl; 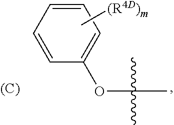
wherein
subscript m is 0, 1, 2, 3, 4, or 5; and
each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein
each R^{4D1} group is independently —NH₂ or —C(O)OR^{4D3},
each R^{4D2} is independently C₁₋₃ alkoxy, and
each R^{4D3} is independently C₁₋₃ alkyl; 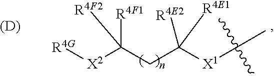
wherein
X¹ and X² are each independently —O— or —N(R^{4H})—;
R^{4E1} and R^{4E2} are each independently H, C₁₋₆ alkyl optionally substituted with 1 to 3 R^{4E3} groups, or C₃₋₆ cycloalkyl, wherein
each R^{4E3} group is independently-C(O)OR^{4E4}, —NH₂, —NHC(O)R^{4E4}, —NHC(O)O—C₁₋₆ alkylene-C₆₋₁₂ aryl, C₃₋₆ cycloalkyl, or C₆₋₁₂ aryl, and
each R^{4F4} group is independently C₁₋₆ alkyl;
or R^{4E1} and R^{4E2} are combined with the atom to which they are attached to form a C₃₋₆ cycloalkyl;
R^{4F1} and R^{4F2} are each H or together are oxo;
R^{4G} is C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or 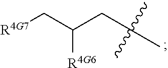
each R^{4G1} is independently —OH, C₁₋₆ alkyl, C₁₋₃ alkoxy, —(CH₂OCH₂)₁₋₅—CH₃, C₁₋₃ haloalkyl, —N(R^{4G8})₂, —C(O)N(R^{4G8})₂, C₃₋₈ cycloalkyl optionally substituted with 1 to 3 R^{4G9}, a 3 to 8 membered heterocyclyl having 1 to 3 heteroatoms selected from N, O and S, optionally substituted with 1 to 3 R^{4G10}, or C₆₋₁₂ aryl;
each R^{4G2} is independently C₁₋₆ alkyl, C₁₋₆ alkoxy, halogen, C₁₋₃ haloalkyl, —OH, —NH₂, or C₆₋₁₂ aryl;
each R^{4G3} is independently C₁₋₆ alkyl, halogen, C₁₋₃ haloalkyl, oxo, —C(O)R^{4G5}, or —C(O)OR^{4G5};
each R^{4G4} is independently C₁₋₆ alkyl, C₇₋₁₈ alkyl or C₃₋₈ cycloalkyl, wherein the C₁₋₆ alkyl is optionally substituted with OH, NH₂, or —NHC(O)OR^{4G5}, and wherein the cycloalkyl is optionally substituted with C₁₋₆ alkyl;
each R^{4G5} is independently C₁₋₆ alkyl;
R^{4G6} and R^{4G7} are each independently H or —OR^{4G11}, wherein at least one of R^{4G6} and R^{4G7} is —OR^{4G11};
each R^{4G8} is independently H or C₁₋₆ alkyl;
each R^{4G9} is independently C₁₋₆ alkyl, halogen, C₁₋₃ haloalkyl, or —NH₂;
each R^{4G10} is independently C₁₋₆ alkyl, C₁₋₃ haloalkyl, or oxo;
each R^{4G11} is independently C₁₀₋₁₈ alkyl or benzyl;
R^{4H} is H;
or R^{4E1} and R^{4H} are combined with the atoms to which they are attached to form a 5 to 6 membered heterocyclyl having 1 to 2 additional heteroatoms selected from N, O and S; and
subscript n is 0 or 1; or
(E) —(OP(O)(OH))₁₋₂—OH; or 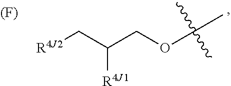
wherein
R^{4J1} and R^{4J2} are each independently H, —OR^{4J3} or —OC(O)R^{4J3}, wherein at least one of R^{4J1} and R^{4J2} is —OR^{4J3} or —OC(O)R^{4J3},
each R^{4J3} is independently C₁₋₁₈ alkyl, C₂₋₆ alkenyl, or benzyl, and at least one R^{4J3} is C₁₀₋₁₈ alkyl;
alternatively, R² and R^{4C} are combined with the atoms to which they are attached to form a six-membered ring, and R¹ is H or —C(O)R^{1A}, wherein R^{1A} is C₁₋₆ alkyl,
with the proviso that when the compound of Formula (Ia) has the formula: 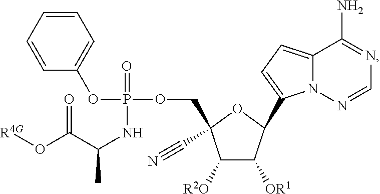
and R^{4G} is ethyl or 2-ethylbutyl, then one of R¹ and R² is —C(O)R^{1A}, or R¹ and R² are combined to form —C(O)— or —C(R^{2A})(R^{2B})—,
with the proviso that the compound of Formula (Ia) does not have the structure: 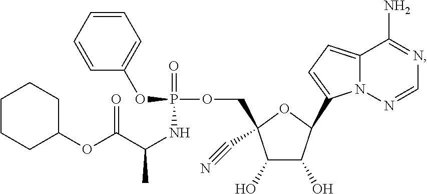 [SMILES: C[C@H](N[P@](=O)(OC[C@@]1(C#N)O[C@@H](c2ccc3c(N)ncnn23)[C@H](O)[C@@H]1O)Oc1ccccc1)C(=O)OC1CCCCC1]
and with the proviso that when the compound of Formula (Ia) has the formula: 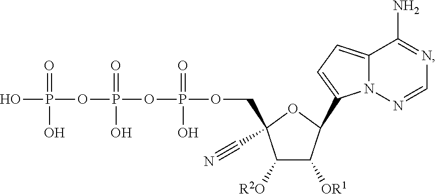
then one of R¹ and R² is —C(O)R^{1A}, or R¹ and R² are combined to form —C(O)— or —C(R^{2A})(R^{2B})—.

2. The compound of claim 1, or a pharmaceutically acceptable salt thereof, wherein
R¹ and R² are each independently H or —C(O)R^{1A}, wherein R^{1A} is methyl, ethyl, n-propyl, iso-propyl, n-butyl, iso-butyl, sec-butyl or t-butyl, wherein at least one of R¹ and R² is H;
or R¹ and R² are combined to form —C(O)—, —C(Me)₂- or —CH(OEt)-.

3. The compound of claim 1, or a pharmaceutically acceptable salt thereof, wherein
R¹ and R² are each independently H or —C(O)R^{1A}, wherein R^{1A} is ethyl, iso-propyl or t-butyl, wherein at least one of R¹ and R² is H;
or R¹ and R² are combined to form —C(O)—, —C(Me)₂- or —CH(OEt)-.

4. The compound of claim 1, or a pharmaceutically acceptable salt thereof, wherein
R³ is NH₂.

5. The compound of claim 1, or a pharmaceutically acceptable salt thereof, wherein
R^{4A} is O.

6. The compound of claim 1, or a pharmaceutically acceptable salt thereof, wherein
R¹ and R² are each independently H or —C(O)R^{1A}, wherein R^{1A} is ethyl, iso-propyl or t-butyl, wherein at least one of R¹ and R² is H;
or R¹ and R² are combined to form —C(O)—, —C(Me)₂- or —CH(OEt)-;
R³ is NH₂; and
R^{4A} is O.

7. The compound of claim 1, or a pharmaceutically acceptable salt thereof, wherein
R^{4B} and R^{4C} are each independently: 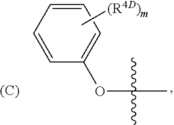
wherein
subscript m is 0, 1, 2, 3, 4, or 5; and
each R^{4D} is independently C₁₋₆ alkyl optionally substituted with 1 to 3 R^{4D1} groups, C₁₋₃ alkoxy optionally substituted with 1 to 3 R^{4D2} groups, —C(O)OR^{4D3}, or —C(O)N(R^{4D3})₂, wherein
each R^{4D1} group is independently —NH₂ or —C(O)OMe,
each R^{4D2} is methoxy, and
each R^{4D3} is independently methyl or ethyl; or 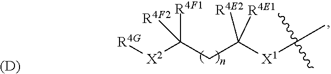
wherein
X¹ and X² are each independently —O— or —NH—;
R^{4E1} is C₁₋₆ alkyl optionally substituted with 1 R^{4E3} group, or C₃₋₆ cycloalkyl, wherein
each R^{4E3} group is independently —C(O)Me, —C(O)O-n-butyl, —C(O)O— pentyl, —NH₂, —NHC(O)Me, —NHC(O)O-benzyl, C₃₋₆ cycloalkyl or phenyl;
R^{4E2} is H;
or R^{4E1} and R^{4E2} are combined with the atom to which they are attached to form a C₃₋₆ cycloalkyl;
R^{4F1} and R^{4F2} are each H or together are oxo;
R^{4G} is C₁₋₆ alkyl optionally substituted with 1 to 3 R^{4G1}, C₇₋₁₈ alkyl, C₃₋₈ cycloalkyl optionally substituted with 1 to 3 R^{4G2}, a 3 to 8 membered heterocyclyl having 1 to 3 heteroatoms selected from N, O and S, optionally substituted with 1 to 3 R^{4G3}, —C(O)R^{4G4}, —C(O)OR^{4G5}, or 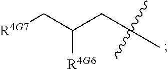
each R^{4G1} is independently —OH, hydroxymethyl, methoxy, —(CH₂OCH₂)₂—CH₃, —CF₃, —N(Me)₂, —C(O)NH₂, C₃₋₈ cycloalkyl optionally substituted with 1 to 2 R^{4G9}, a 3 to 8 membered heterocyclyl having 1 to 3 heteroatoms selected from N, O and S, optionally substituted with 1 to 2 R^{4G10}, or phenyl;
each R^{4G2} is independently methyl, ethyl, n-propyl, iso-propyl, n-butyl, iso-butyl, sec-butyl, t-butyl, methoxy, F, Cl, Br, CF₃, —NH₂, or phenyl;
each R^{4G3} is independently methyl, ethyl, F, Cl, CF₃, CH₂CF₃, oxo, —C(O)Me, or —C(O)O-t-butyl;
each R^{4G4} is independently C₁₋₆ alkyl, C₇₋₁₈ alkyl or C₃₋₇ cycloalkyl, wherein the C₁₋₆ alkyl is optionally substituted with OH, NH₂, or —NHC(O)O-t-butyl, and wherein the cycloalkyl is optionally substituted with methyl, ethyl, n-propyl, iso-propyl, n-butyl, iso-butyl, sec-butyl, or t-butyl;
each R^{4G5} is independently methyl, ethyl, n-propyl, iso-propyl, n-butyl, iso-butyl, sec-butyl, or t-butyl;
R^{4G6} and R^{4G7} are each independently H or —OR^{4G11}, wherein at least one of R^{4G6} and R^{4G7} is —OR^{4G11};
each R^{4G9} is independently methyl, CF₃, or —NH₂;
each R^{4G10} is independently methyl, CF₃, CH₂CF₃, or oxo; and
each R^{4G11} is independently hexadecane, octadecane or benzyl.

8. The compound of claim 1, having Formula (Ib): 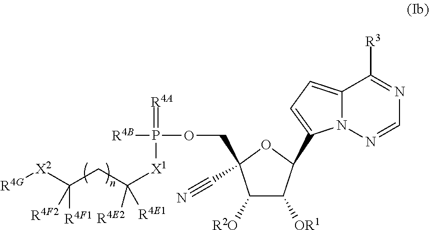
or a pharmaceutically acceptable salt thereof.

9. The compound of claim 1, having Formula (Ic): 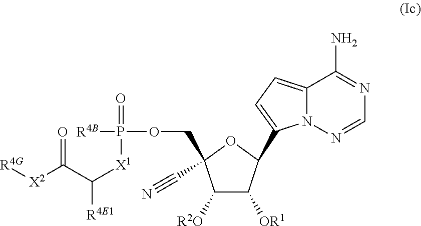
or a pharmaceutically acceptable salt thereof.

10. The compound of claim 1, or a pharmaceutically acceptable salt thereof, wherein
R^{4B} is: 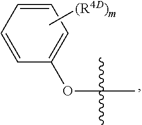
wherein
subscript m is 1; and
R^{4D} is independently methyl, ethyl, n-propyl, or tert-butyl, each optionally substituted with 1 to 3 R^{4D1} groups, wherein each R^{4D1} group is independently —NH₂ or —C(O)OMe, or
R^{4D} is methoxy, ethoxy, or propoxy, each optionally substituted with methoxy, or
R^{4D} is —C(O)OMe, —C(O)OEt or —C(O)N(Me)₂; and
R^{4C} is: 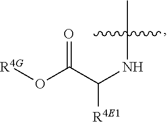
wherein
R^{4E1} is methyl, ethyl, n-propyl, iso-propyl, n-butyl, iso-butyl, sec-butyl, t-butyl, n-pentane, neopentane, or n-hexane, each optionally substituted with 1 R^{4E3} group wherein each R^{4E3} group is independently —C(O)Me, —C(O)O-n-butyl, —C(O)O-pentyl, —NH₂, —NHC(O)Me, or —NHC(O)O— benzyl, or
R^{4E1} is cyclopropyl, cyclopropylmethyl, cyclobutyl, cyclobutylmethyl, cyclopentyl, cyclopentylmethyl, cyclohexyl, or cyclohexylmethyl, or
R^{4E1} is benzyl.

11. The compound of claim 1, having Formula (Id): 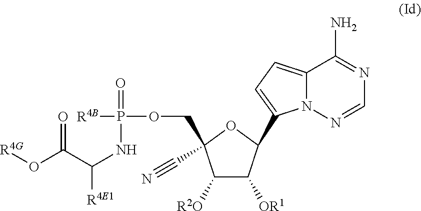
or a pharmaceutically acceptable salt thereof.

12. The compound of claim 1, having Formula (Ie): 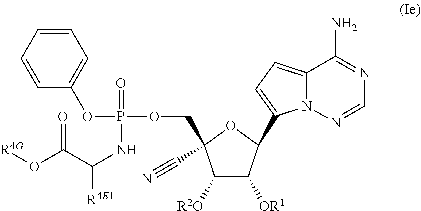
or a pharmaceutically acceptable salt thereof.

13. The compound of claim 1, having Formula (If): 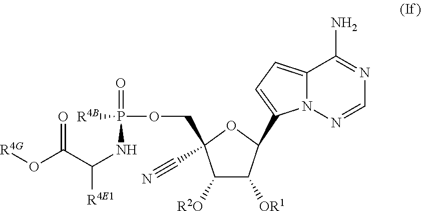
or a pharmaceutically acceptable salt thereof.

14. The compound of claim 1, having Formula (Ig): 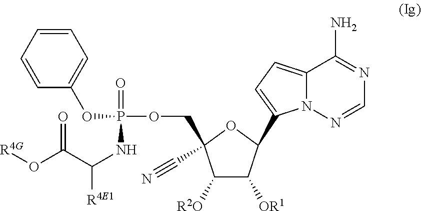
or a pharmaceutically acceptable salt thereof.

15. The compound of claim 1, having Formula (Ih): 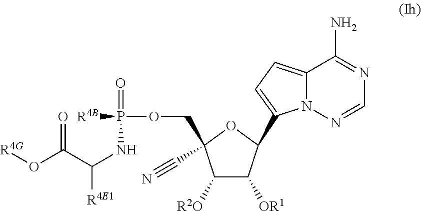
or a pharmaceutically acceptable salt thereof.

16. The compound of claim 1, having Formula (Ii): 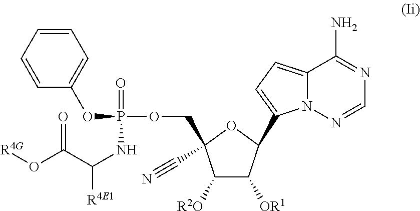
or a pharmaceutically acceptable salt thereof.

17. The compound of claim 1, or a pharmaceutically acceptable salt thereof, wherein R^{4C} is:

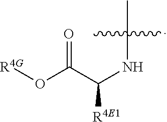

18. The compound of claim 1, or a pharmaceutically acceptable salt thereof, wherein R^{4C} is:

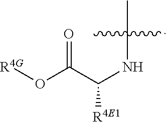

19. The compound of claim 1, or a pharmaceutically acceptable salt thereof, wherein
R^{4G} is methyl, ethyl, n-propyl, iso-propyl, n-butyl, iso-butyl, sec-butyl, t-butyl, n-pentane, neopentane, n-hexane, 2,2-dimethylbutyl, 3,3-dimethylbutyl, 2-ethyl-butyl, heptane, octane, nonane, decane, undecane, dodecane, pentadecane, hexadecane, or octadecane, each optionally substituted with 1 to 2 R^{4G1} wherein each R^{4G1} is independently —OH, hydroxymethyl, methoxy, —(CH₂OCH₂)₂—CH₃, —CF₃, —N(Me)₂, or —C(O)NH₂.

20. The compound of claim 1, or a pharmaceutically acceptable salt thereof, wherein
R^{4G} is cyclopropyl, cyclobutyl, cyclopentyl, cyclohexyl, cycloheptyl, or cyclooctyl, each optionally substituted with 1 to 2 R^{4G2} wherein each R^{4G2} is independently methyl, ethyl, n-propyl, iso-propyl, n-butyl, iso-butyl, sec-butyl, t-butyl, OMe, F, CF₃, —NH₂, or phenyl.

21. The compound of claim 1, or a pharmaceutically acceptable salt thereof, wherein
R^{4G} is cyclopropylmethyl, cyclobutylmethyl, cyclopentylmethyl, cyclohexylmethyl, cycloheptylmethyl, or cyclooctylmethyl, each optionally substituted with 1 to 2 R^{4G2} wherein each R^{4G2} is independently methyl, CF₃, or —NH₂.

22. The compound of claim 1, or a pharmaceutically acceptable salt thereof, wherein
R^{4G} is pyrrolidine, piperidine, azepane, quinuclidine, oxetane, tetrahydrofuran, tetrahydropyran, morpholine, or 1,3-dioxol, each optionally substituted with 1 to 2 R^{4G3} wherein each R^{4G3} is independently methyl, ethyl, F, CH₂CF₃, oxo, —C(O)Me, or —C(O)O-t-butyl.

23. The compound of claim 1, or a pharmaceutically acceptable salt thereof, wherein
R^{4G} is piperidinemethyl, quinuclidinemethyl, oxetanemethyl, tetrahydrofuranmethyl, tetrahydropyranmethyl, morpholinemethyl, 2-morpholine-ethyl, 3-morpholine-propyl, or 1,3-dioxolmethyl, each optionally substituted with 1 to 2 R^{4G10} wherein each R^{4G10} is independently methyl, CH₂CF₃, or oxo.

24. The compound of claim 1, or a pharmaceutically acceptable salt thereof, wherein
R^{4G} is benzyl.

25. The compound of claim 1, or a pharmaceutically acceptable salt thereof, wherein
R^{4G} is —C(O)R^{4G4}, wherein
R^{4G4} is
C₁₋₆ alkyl selected from the group consisting of methyl, ethyl, n-propyl, iso-propyl, n-butyl, iso-butyl, sec-butyl, t-butyl, n-pentane, neopentane, n-hexane, 2,2-dimethylbutyl, 3,3-dimethylbutyl, and 2-ethyl-butyl,
C₇₋₁₈ alkyl selected from the group consisting of heptane, octane, nonane, decane, undecane, dodecane, pentadecane, hexadecane, and octadecane, or
C₃₋₈ cycloalkyl selected from the group consisting of cyclopropyl, cyclobutyl, cyclopentyl, or cyclohexyl,
wherein each C₁₋₆ alkyl is optionally substituted with OH, NH₂, or —NHC(O)O-t-butyl, and
wherein each C₃₋₈ cycloalkyl is optionally substituted with methyl.

26. The compound of claim 1, or a pharmaceutically acceptable salt thereof, wherein
R^{4G} is —C(O)OR^{4G5}, wherein R^{4G5} is methyl, ethyl, n-propyl, iso-propyl, n-butyl, iso-butyl, sec-butyl, or t-butyl.

27. The compound of claim 1, or a pharmaceutically acceptable salt thereof, wherein
R^{4G} is 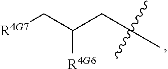
wherein
R^{4G6} and R^{4G7} are each independently H or —OR^{4G11}, wherein
at least one of R^{4G6} and R^{4G7} is —OR^{4G11}, and
each R^{4G11} is independently hexadecane, octadecane or benzyl.

28. The compound of claim 1, or a pharmaceutically acceptable salt thereof, wherein R^{4G} is methyl, ethyl, n-propyl, iso-propyl, n-butyl, pentyl, neopentyl, hexyl, 2,2-dimethylbutyl, 3,3-dimethylbutyl, 2-ethyl-butyl, octyl, dodecyl, hexadecyl, octadecyl, cyclobutyl, cyclopentyl, cyclohexyl, cycloheptyl, cyclooctyl,

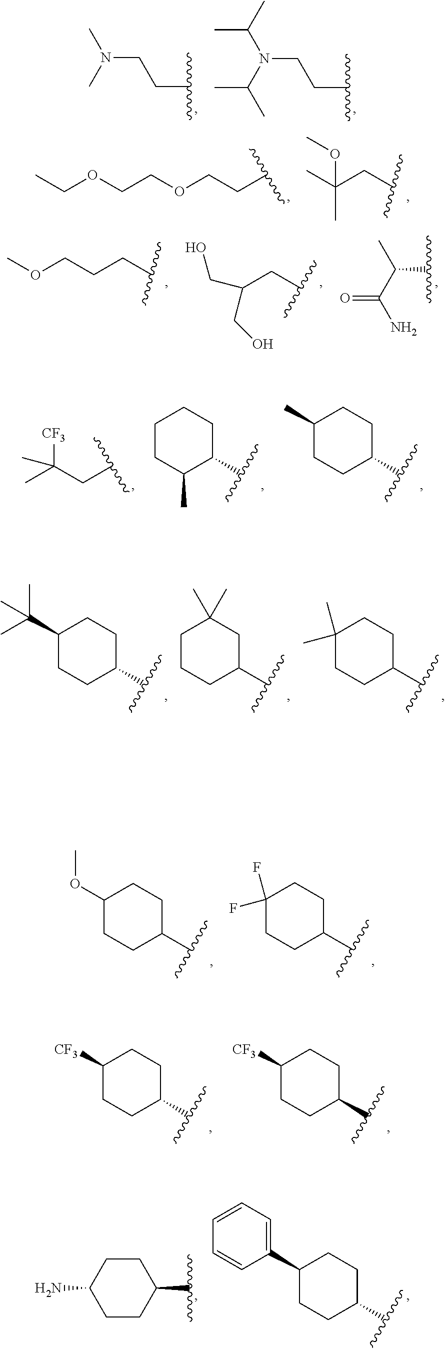

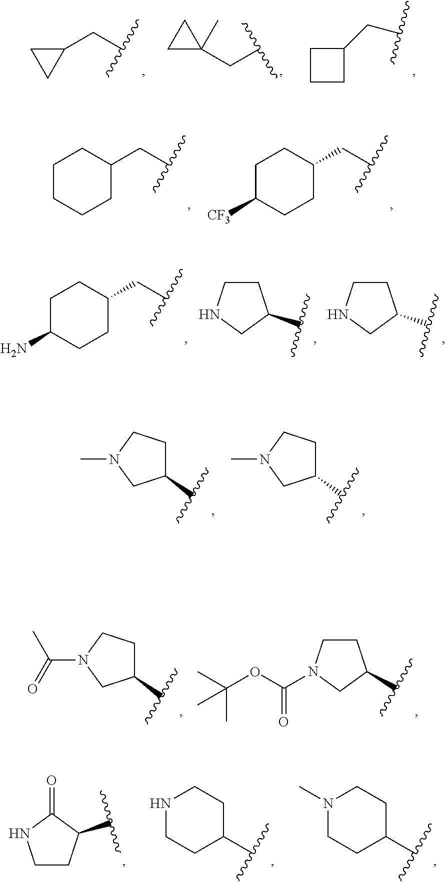

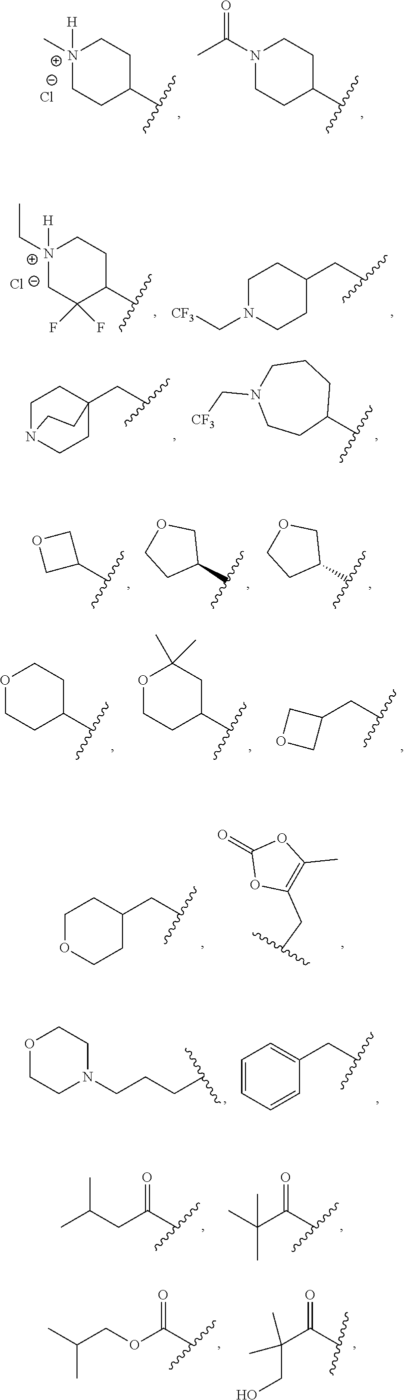

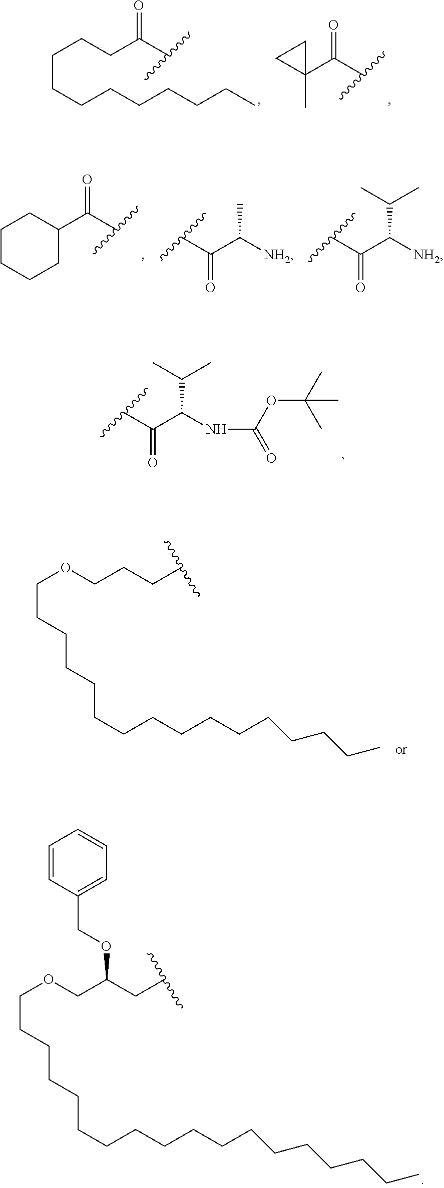

29. A compound, or a pharmaceutically acceptable salt thereof, of Table 1A, Table 1B, Table 1C, Table 1D, Table 1E, Table 1F, Table 1G, Table 1H, Table 1I or Table 1J.

30. A compound, or a pharmaceutically acceptable salt thereof, having the structure of:

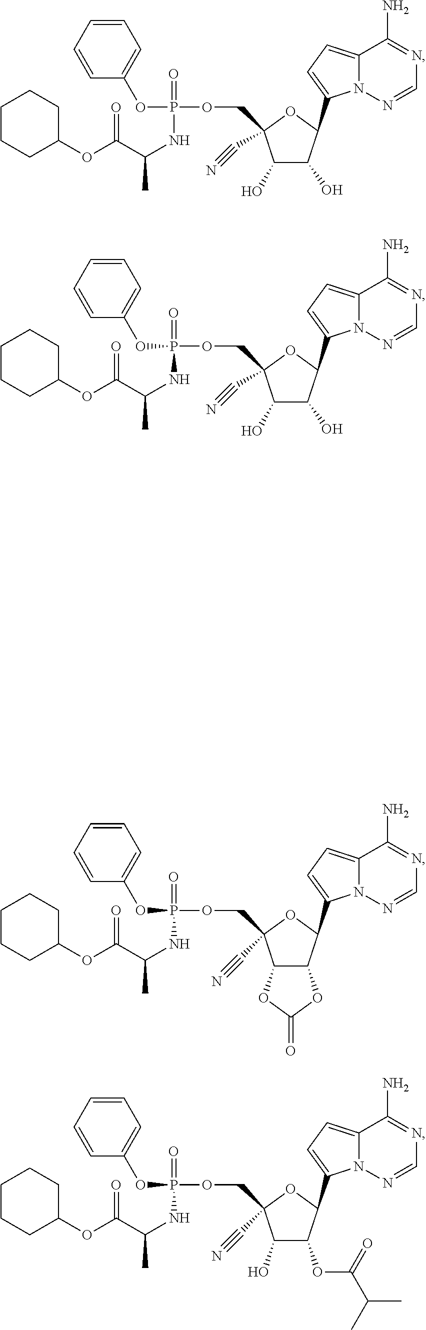

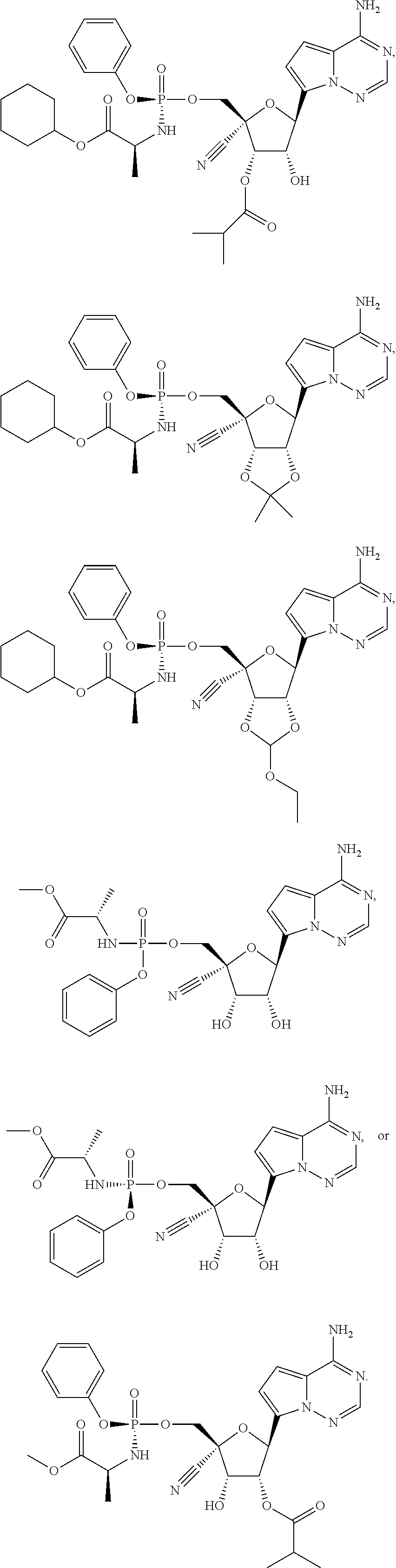

31. A compound, or a pharmaceutically acceptable salt thereof, having the structure of:

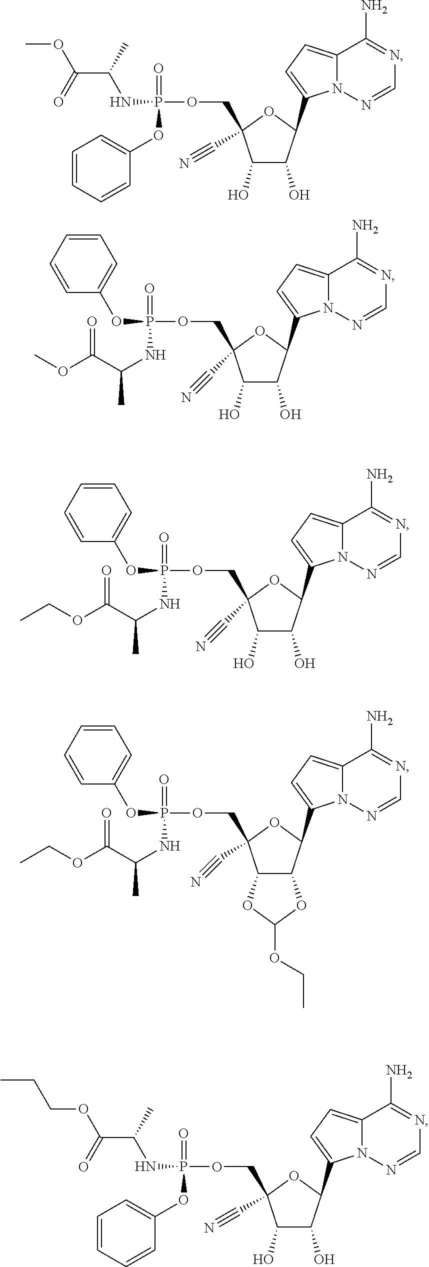

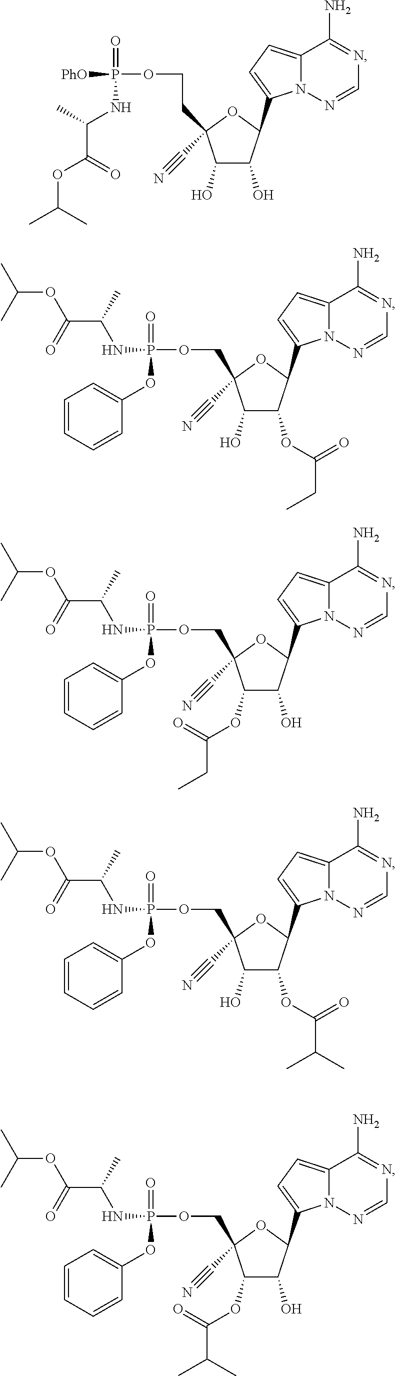

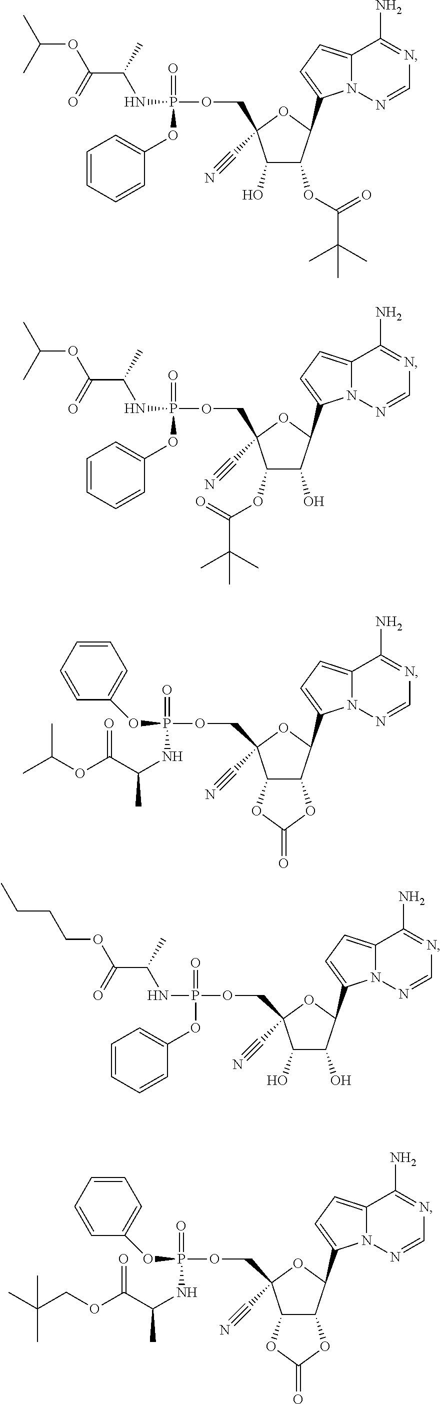

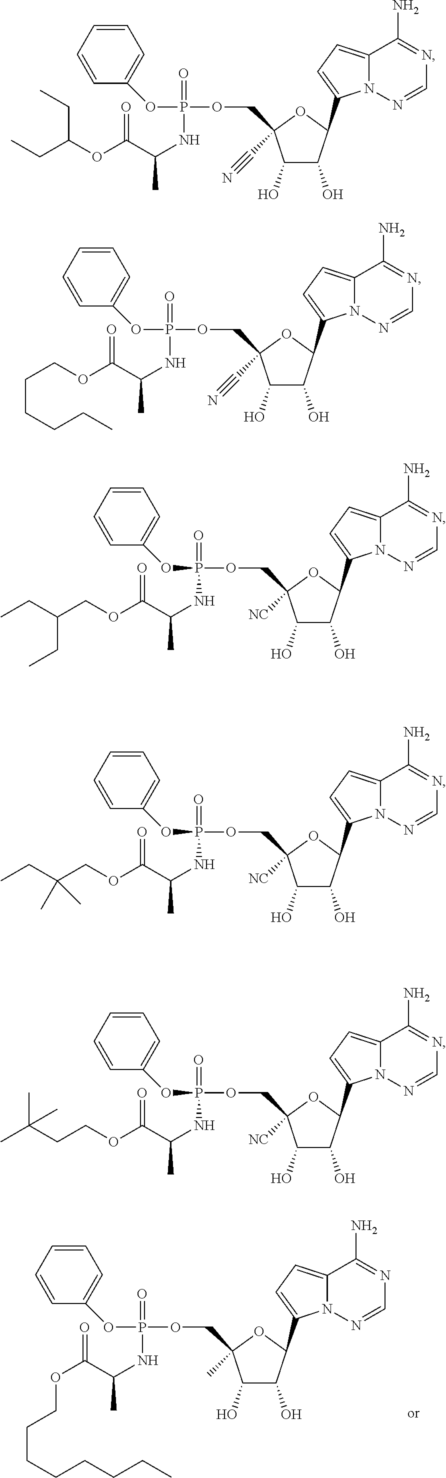

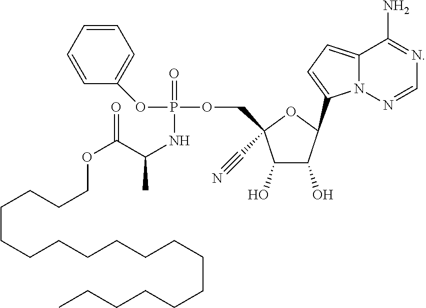 [SMILES: CCCCCCCCCCCCCCCCCCOC(=O)[C@H](C)NP(=O)(OC[C@@]1(C#N)O[C@@H](c2ccc3c(N)ncnn23)[C@H](O)[C@@H]1O)Oc1ccccc1]

32. A pharmaceutical formulation comprising a pharmaceutically effective amount of a compound of claim 1, or a pharmaceutically acceptable salt thereof, and a pharmaceutically acceptable carrier or excipient.

33. A method of treating a Pneumoviridae virus infection in a human in need thereof, the method comprising administering to the human a therapeutically effective amount of a compound of claim 1 or a pharmaceutically acceptable salt thereof.

34.-35. (canceled)

36. A method of treating a Picornaviridae virus infection in a human in need thereof, the method comprising administering to the human a therapeutically effective amount of a compound of claim 1 or a pharmaceutically acceptable salt thereof.

37. (canceled)

38. A method of treating a Flaviviridae virus infection in a human in need thereof, the method comprising administering to the human a therapeutically effective amount of a compound of claim 1 or a pharmaceutically acceptable salt thereof.

39.-43. (canceled)

44. A method of treating a Filoviridae virus infection in a human in need thereof, the method comprising administering to the human a therapeutically effective amount of a compound of claim 1 or a pharmaceutically acceptable salt thereof.

45.-80. (canceled)

81. A method for the treatment or prophylaxis of an exacerbation of a respiratory condition by a viral infection in a human in need thereof, the method comprising administering to the human a therapeutically effective amount of a compound of claim 1, or a pharmaceutically acceptable salt thereof, wherein the respiratory condition is chronic obstructive pulmonary disease.

82.-88. (canceled)
